(19) United States
(12) Patent Application Publication
(10) Pub. No.: US 2016/0297833 A1
(43) Pub. Date: Oct. 13, 2016

(54) THIENOPYRIMIDINES AS MKNK1 AND MKNK2 INHIBITORS

(71) Applicant: BAYER PHARMA AKTIENGESELLSCHAFT, Berlin (DE)

(72) Inventors: Ulrich KLAR, Berlin (DE); Lars WORTMANN, Berlin (DE); Georg KETTSCHAU, Berlin (DE); Keith GRAHAM, Berlin (DE); Anja RICHTER, Berlin (DE); Philip LIENAU, Berlin (DE); Florian PUEHLER, Wellesley, MA (US); Kirstin PETERSEN, Berlin (DE); Franziska SIEGEL, Berlin (DE); Detlev SÜLZLE, Berlin (DE)

(21) Appl. No.: 15/037,949

(22) PCT Filed: Nov. 17, 2014
(86) PCT No.: PCT/EP2014/074722

§ 371 Date: May 19, 2016

(30) Foreign Application Priority Data
Nov. 20, 2013 (EP) 13193665.0
Jun. 27, 2014 (EP) 14174731.1

(51) Int. Cl. C07D 495/04; A61K 31/655; A61K 31/5377; C07D 519/00; A61K 31/519; A61K 45/06

(52) U.S. Cl. CPC C07D 495/04; A61K 31/519; A61K 45/06; A61K 31/5377; C07D 519/00; A61K 31/655

(57) ABSTRACT

The present invention relates to substituted thienopyrimidine compounds of general formula (I) as described and defined herein, to methods of preparing said compounds, to intermediate compounds useful for preparing said compounds, to pharmaceutical compositions and combinations comprising said compounds and to the use of said compounds for manufacturing a pharmaceutical composition for the treatment or prophylaxis of a disease, in particular of a hyper-proliferative and/or angiogenesis disorder, as a sole agent or in combination with other active ingredients.

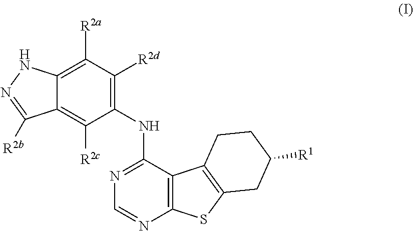

The present invention relates to substituted thienopyrimidine compounds of general formula (I) as described and defined herein, to methods of preparing said compounds, to intermediate compounds useful for preparing said compounds, to pharmaceutical compositions and combinations comprising said compounds and to the use of said compounds for manufacturing a pharmaceutical composition for the treatment or prophylaxis of a disease, in particular of a hyper-proliferative and/or angiogenesis disorder, as a sole agent or in combination with other active ingredients.

### BACKGROUND OF THE INVENTION

The present invention relates to chemical compounds that inhibit MKNK1 kinase (also known as MAP Kinase interacting Kinase, Mnk1) and/or MKNK2 kinase (also known as MAP Kinase interacting Kinase, Mnk2). Human MKNKs comprise a group of four proteins encoded by two genes (Gene symbols: MKNK1 and MKNK2) by alternative splicing. The b-forms lack a MAP kinase-binding domain situated at the C-terminus. The catalytic domains of the MKNK1 and MKNK2 are very similar and contain a unique DFD (Asp-Phe-Asp) motif in subdomain VII, which usually is DFG (Asp-Phe-Gly) in other protein kinases and suggested to alter ATP binding [Jauch et al., Structure 13, 1559-1568, 2005 and Jauch et al., EMBO J25, 4020-4032, 2006]. MKNK1a binds to and is activated by ERK and p38 MAP Kinases, but not by JNK1. MKNK2a binds to and is activated only by ERK. MKNK1b has low activity under all conditions and MKNK2b has a basal activity independent of ERK or p38 MAP Kinase. [Buxade M et al., Frontiers in Bioscience 5359-5374, May 1, 2008]

MKNKs have been shown to phosphorylate eukaryotic initiation factor 4E (eIF4E), heterogeneous nuclear RNA-binding protein A1 (hnRNP A1), polypyrimidine-tract binding protein-associated splicing factor (PSF), cytoplasmic phospholipase A2 (cPLA2) and Sprouty 2 (hSPRY2) [Buxade M et al., Frontiers in Bioscience 5359-5374, May 1, 2008].

eIF4E is an oncogene that is amplified in many cancers and is phosphorylated exclusively by MKNKs proteins as shown by KO-mouse studies [Konicek et al., Cell Cycle 7:16, 2466-2471, 2008; Ueda et al., Mol Cell Biol 24, 6539-6549, 2004]. eIF4E has a pivotal role in enabling the translation of cellular mRNAs. eIF4E binds the 7-methylguanosine cap at the 5′ end of cellular mRNAs and delivers them to the ribosome as part of the eIF4F complex, also containing eIF4G and eIF4A. Though all capped mRNAs require eIF4E for translation, a pool of mRNAs is exceptionally dependent on elevated eIF4E activity for translation. These so-called “weak mRNAs” are usually less efficiently translated due to their long and complex 5′ UTR region and they encode proteins that play significant roles in all aspects of malignancy including VEGF, FGF-2, c-Myc, cyclin D1, survivin, BCL-2, MCL-1, MMP-9, heparanase, etc. Expression and function of eIF4E is elevated in multiple human cancers and directly related to disease progression [Konicek et al., Cell Cycle 7:16, 2466-2471, 2008].

MKNK1 and MKNK2 are the only kinases known to phosphorylate eIF4E at Ser209. Overall translation rates are not affected by eIF4E phosphorylation, but it has been suggested that eIF4E phosphorylation contributes to polysome formation (i.e. multiple ribosome on a single mRNA) that ultimately enables more efficient translation of “weak mRNAs” [Buxade M et al., Frontiers in Bioscience 5359-5374, May 1, 2008]. Alternatively, phosphorylation of eIF4E by MKNK proteins might facilitate eIF4E release from the 5′ cap so that the 48S complex can move along the “weak mRNA” in order to locate the start codon [Blagden S P and Willis A E, Nat Rev Clin Oncol. 8(5):280-91, 2011]. Accordingly, increased eIF4E phosphorylation predicts poor prognosis in non-small cell lung cancer patients [Yoshizawa et al., Clin Cancer Res. 16(1):240-8, 2010]. Further data point to a functional role of MKNK1 in carcinogenesis, as overexpression of constitutively active MKNK1, but not of kinase-dead MKNK1, in mouse embryo fibroblasts accelerates tumor formation [Chrestensen C. A. et al., Genes Cells 12, 1133-1140, 2007]. Moreover, increased phosphorylation and activity of MKNK proteins correlate with overexpression of HER2 in breast cancer [Chrestensen, C. A. et al., J. Biol. Chem. 282, 4243-4252, 2007]. Constitutively active, but not kinase-dead, MKNK1 also accelerated tumor growth in a model using Ep-Myc transgenic hematopoietic stem cells to produce tumors in mice. Comparable results were achieved when an eIF4E carrying a S209D mutation was analyzed. The S209D mutation mimicks a phosphorylation at the MKNK1 phosphorylation site. In contrast, a non-phosphorylatable form of eIF4E attenuated tumor growth [Wendel H G, et al., Genes Dev. 21(24):3232-7, 2007]. A selective MKNK inhibitor that blocks eIF4E phosphorylation induces apoptosis and suppresses proliferation and soft agar growth of cancer cells in vitro. This inhibitor also suppresses outgrowth of experimental B16 melanoma pulmonary metastases and growth of subcutaneous HCT116 colon carcinoma xenograft tumors without affecting body weight [Konicek et al., Cancer Res. 71(5):1849-57, 2011]. In summary, eIF4E phosphorylation through MKNK protein activity can promote cellular proliferation and survival and is critical for malignant transformation. Inhibition of MKNK activity may provide a tractable cancer therapeutic approach.

Substituted thienopyrimidine compounds have been disclosed in prior art for the treatment or prophylaxis of different diseases:

WO2013/106535 (Nimbus Iris, Inc.) discloses tricyclic thienopyrimidine derivatives as inhibitors of IRAK protein kinases, for the treatment of a variety of diseases, including inflammatory disorders, neurodegenerative disorders and cancer. The compounds claimed feature a saturated or partially unsaturated but not aromatic ring system A attached to position 4 of the pyrimidine ring, which typically is a substituted cyclohexane in the explicit example compounds disclosed, rendering said compounds different from the compounds of the present invention.

WO2010/006032(A1) (Duquesne University of the Holy Spirit) addresses tricyclic compounds as antimitotic agents. According to the general formula of claim 1, the tricycles inter alia comprise 5,6,7,8-tetrahydrobenzo[1]thieno[2,3-d]pyrimidines that may carry substituents at the carbocycle and one aromatic or heteroaromatic moiety at an optional 4-amino group. Furthermore, they may be unsubstituted at position 2 in the pyrimidine ring. However, the examples provided clearly differ from the compounds of the present invention. While the vast majority contains the C6 carbocycle completely unsaturated as aromatic ring, only two examples show a tetrahydrobenzo substructure in combination with a 4-amino group and in both cases the latter is bisubstituted by a phenyl and a methyl group. Furthermore, the specified compounds are with no exception pyrimidin-2-amines or 2-methyl-pyrimidines.

JP2007084494 (Oncorex Inc.) relates to PIM-1 inhibitors. One claim comprises 5,6,7,8-tetrahydrobenzo[1]thieno[2,3-d]pyrimidin-4-amines that can be monosubstituted at the amino group by optionally substituted phenyl. However, the optional substituents of phenyl are restricted to hydroxy, alkoxy or alkenyloxy. The tricyclic core does not show further substitutions. The only example of a direct substitution at the 4-amino group by phenyl is compound VII-2 with meta-methoxyphenyl.

WO2002/088138(A1) (Bayer Pharmaceuticals Corporation) relates to PDE7b inhibitors and comprises 5,6,7,8-tetrahydrobenzo[1]thieno[2,3-d]pyrimidin-4-amines where the carbocycle and the 4-amino group may be optionally substituted by a wide range of substituents. The respective oxa, thia or aza analoga at position 7 with no further substituents at that ring are also claimed, the sulphur may be oxidized to sulphone and the nitrogen can be substituted. However, pyrid-4-yl in the 5,6,7,8-tetrahydrobenzo series and 3,4-dichlorophenyl and indazol-5-yl in the 6,9-dihydro-7H-pyrano series are the only examples with direct aromatic substitution at the 4-amino group.

WO2005/010008(A1) (Bayer Pharmaceuticals Corporation) discloses 5,6,7,8-tetrahydrobenzo[1]thieno[2,3-d]pyrimidin-4-amines as proliferation inhibitors of A431 and BT474 cells which are model cell lines used in biomedical research. More specifically, A431 and BT474 cells are used in studies of the cell cycle and cancer-associated cell signalling pathways since they express abnormally high levels of the epidermal growth factor receptor (EGFR) and HER2, respectively. Substitution at the 4-amino group is limited to monosubstitution by either optionally substituted phenyl or optionally substituted indazolyl. The carbocycle may be substituted one or two times at position 7 by optionally substituted alkyl or alkenyl, by substituted carbonyl, hydroxy, optionally substituted amino or may be linked to the nitrogen of one or two saturated six membered rings optionally bearing a second heteroatom. Regarding the aromatic substituents at the 4-amino group, disclosed examples cover phenyl with a broad range of substituents and some indazol-5-yls but all are substituted at the nitrogen at position 1. Furthermore, all examples show an alkyl group in position 7 that is terminally further substituted by an amino group or hydroxyl group or in case of synthetic intermediates also by an ester function. Furthermore, as shown hereinafter, the compounds disclosed in WO 2005/010008 A1 are potent EGFR inhibitors but less effective MKNK inhibitors whereas the compounds of the present invention are potent MKNK inhibitors and less effective EGFR inhibitors.

WO2009/134658(A1) (National Health Research Institutes) relates to inhibitors of Aurora kinase. The patent application generically covers tricyclic thieno[2,3-d]pyrimidin-4-amines with the third ring fused to the thiophene subunit. However, an optional aryl or heteroaryl substituent at the 4-amino group must carry a side chain involving a carbonyl, thiocarbonyl or iminomethylene group. The vast majority of more than 250 examples is formed by bicyclic 6,7-dihydrofuro[3,2-d]pyrimidin-4-amines that show in 4 cases a direct aromatic substitution at the 4-amino group but additionally substitution by two phenyl groups at the dihydrofuro subunit. None of the very few examples for tricyclic compounds shows direct substitution by an aromatic moiety at the 4-amino group.

WO2006/136402(A1) and WO2007/059905(A2) (Develogen AG) disclose thienopyrimidin-4-amines and their use for the prophylaxis and/or treatment of diseases which can be influenced by the inhibition of the kinase activity of Mnk1 and/or Mnk2. The 4-amino-group is substituted by a substituted phenyl group. The WO publications do not disclose any biological data.

WO2010/023181(A1), WO2011/104334(A1), WO2011/104337(A1), WO2011/104338(A1) and WO2011/104340(A1) (Boehringer Ingelheim) relate to thienopyrimidin-4-amines for the prophylaxis and/or treatment of diseases which can be influenced by the inhibition of the kinase activity of Mnk1 and/or Mnk2. In case of the disclosed thienopyrimidin-4-amines there is no tetrahydrobenzo ring fused to the thienopyrimidine core. Additionally, the 4-amino group does not carry an indazol-5-yl substituent. In case of the compounds disclosed in WO2010/023181(A1) the IC₅₀ values vary between 0.035 μM and 0.68 μM with respect Mnk1, and between 0.006 μM and 0.56 μM with respect to Mnk2. In case of the compounds disclosed in WO2011/104334(A1) the IC₅₀ values vary between 1 nM and 9700 nM with respect to Mnk2. In case of the compounds disclosed in WO2011/104337(A1) the IC₅₀ values vary between 2 nM and 8417 nM with respect to Mnk2. In case of the compounds disclosed in WO2011/104338(A1) the IC₅₀ values vary between 8 nM and 58 nM with respect to Mnk2. In case of the compounds disclosed in WO2011/104340(A1) the IC₅₀ values vary between 3 nM and 5403 nM with respect to Mnk2. All WO-publications contain the statement that the compounds described therein show improved solubility, are highly selective and show improved metabolic stability when compared to the compounds disclosed in WO2006/136402(A1) and WO2007/059905(A2) (Develogen AG, see above). However, besides the IC₅₀ values discussed in this paragraph, there are no more data proving this statement.

WO2013/174744(A1) relates to substituted thienopyrimidine compounds as inhibitors of MKNK1 kinase. The general formula (I) of WO2013/174744(A1) generically covers some of the compounds of the present invention. WO2013/174744(A1) was disclosed to the public after the first filing of a patent application for the present invention. In WO2013/174744(A1) the absolute stereochemical configuration of the carbon atom to which the R¹ substitute is bound is not specified. It was found that in case of many of the compounds specifically described in WO2013/174744(A1) the inhibitory activities of the S and the R enantiomers do not differ very much. Surprisingly it was found that for R¹ being a tertiary amide moiety, the activity of the S enantiomer is much higher than the activity of the R enantiomer.

So, the state of the art described above does not describe the specific substituted thienopyrimidine compounds of general formula (I) of the present invention as defined herein or a tautomer, an N-oxide, a hydrate, a solvate, or a salt thereof, or a mixture of same, as described and defined herein, and as hereinafter referred to as “compounds of the present invention”, or their pharmacological activity.

It has now been found, and this constitutes the basis of the present invention, that said compounds of the present invention have surprising and advantageous properties.

In particular, said compounds of the present invention have surprisingly been found to effectively inhibit MKNK1 kinase and may therefore be used for the treatment or prophylaxis of diseases of uncontrolled cell growth, proliferation and/or survival, inappropriate cellular immune responses, or inappropriate cellular inflammatory responses or diseases which are accompanied with uncontrolled cell growth, proliferation and/or survival, inappropriate cellular immune responses, or inappropriate cellular inflammatory responses, particularly in which the uncontrolled cell growth, proliferation and/or survival, inappropriate cellular immune responses, or inappropriate cellular inflammatory responses is mediated by MKNK1 kinase, such as, for example, haematological tumours, solid tumours, and/or metastases thereof, e.g. leukaemias and myelodysplastic syndrome, malignant lymphomas, head and neck tumours including brain tumours and brain metastases, tumours of the thorax including non-small cell and small cell lung tumours, gastrointestinal tumours, endocrine tumours, mammary and other gynaecological tumours, urological tumours including renal, bladder and prostate tumours, skin tumours, and sarcomas, and/or metastases thereof.

Additionally, the compounds of the present invention show higher kinase inhibition selectivity and/or better performance in cellular assays than the MKNK inhibitors disclosed in prior art.

### SUMMARY OF THE INVENTION

The present invention covers compounds of general formula (I):

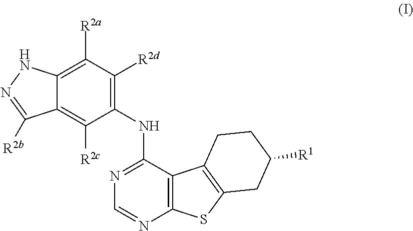

in which:
R¹ represents —C(═O)N(R³)R⁴;
R^{2a} represents a hydrogen atom;
R^{2b} represents a hydrogen atom;
R^{2c} represents a hydrogen atom or a halogen atom;
R^{2d} represents a hydrogen atom, a halogen atom, or a group selected from: cyano-, —OR⁵, —SR⁶, —S(═O)₂R⁶, —S(═O)(═NH)R⁶, —N(H)R⁷, —N(R⁶)R⁷, —N(R⁶)R¹¹;
R³ represents a group selected from:
   C₁-C₆-alkyl-, C₁-C₆-alkoxy-, C₃-C₆-alkenyl-, C₃-C₆-alkynyl-, —(CH₂)_{q}—(C₃-C₇-cycloalkyl), —(CH₂)ₚ—O—(C₃-C₇-cycloalkyl), —(CH₂)_{q}—(C₄-C₇-cycloalkenyl), —(CH₂)ₚ—O—(C₄-C₇-cycloalkenyl), —(CH₂)_{q}— (3- to 10-membered heterocycloalkyl), —(CH₂)ₚ—O— (3- to 10-membered heterocycloalkyl), —(CH₂)_{q}— (4- to 10-membered heterocycloalkenyl), —(CH₂)ₚ—O— (4- to 10-membered heterocycloalkenyl), —(CH₂)_{q}-aryl, —(CH₂)ₚ—O-aryl, —(CH₂)_{q}-heteroaryl, —(CH₂)ₚ—O-heteroaryl, —S(═O)₂—R⁶;
   wherein said C₁-C₆-alkyl-group is optionally substituted one, two or three times, identically or differently, with a group selected from:
   halo-, C₁-C₃-alkoxy-, HO—, —N(R⁸)R⁹;
R⁴ represents a C₁-C₄-alkyl-group;
   wherein said C₁-C₄-alkyl- group is optionally substituted one, two or three times, identically or differently, with a halogen atom or a group selected from: HO—, C₁-C₃-alkoxy-, —CN, —N(R⁸)R⁹, —N(R⁷)R⁸, —C(═O)N(R⁸)R⁹, —C(═O)N(R⁷)R⁸;
Or
N(R³)R⁴ together
   represent a 3- to 10-membered heterocycloalkyl- or 4- to 10-membered heterocycloalkenyl group;
   wherein said 3- to 10-membered heterocycloalkyl- group is optionally substituted one, two or three times, identically or differently, with a halogen atom or a group selected from: —(CH₂)_{q}—OH, —N(R⁷)R⁸, —N(R⁸)R⁹, C₁-C₃-alkyl-, —CN, —C(═O)R¹⁰, —C(═O)N(R⁸)R⁹, —(CH₂)_{q}-aryl, —(CH₂)_{q}-heteroaryl, —(C₁-C₃-alkyl)-N(R⁸)R⁹;
R⁵ represents a hydrogen atom or a group selected from:
   C₁-C₅-alkyl-, —(CH₂)ₘ—(C₃-C₇-cycloalkyl), —(CH₂)ₘ— (3- to 10-membered heterocycloalkyl);
   wherein said C₁-C₅-alkyl- group is optionally substituted one, two or three times, identically or differently, with a halogen atom or a group selected from: cyano, —N(R⁸)R⁹, —N(R⁸)C(═O)R¹⁰, -azido, phenyl-;
   wherein said C₃-C₇-cycloalkyl- group and 3- to 10-membered heterocycloalkyl- group are optionally substituted, one time, with a group selected from: cyano, —N(R⁸)R⁹, —C(═O)—O—R⁹;
R⁶ represents a hydrogen atom or a C₁-C₄-alkyl- group;
R⁷ represents a C₁-C₄-alkyl-, C₃-C₄-alkenyl- or C₁-C₃-alkoxy- group;
   wherein said C₁-C₄-alkyl- is optionally substituted once with —OH or —N(R⁸)R⁹;
or
N(R⁶)R⁷ together
   represent a 3- to 10-membered heterocycloalkyl- or 4- to 10-membered heterocycloalkenyl group;
   wherein said 3- to 10-membered heterocycloalkyl- group is optionally substituted once with —N(R⁸)R⁹;
R⁸ represents a hydrogen atom or a C₁-C₄-alkyl- group;
R⁹ represents a hydrogen atom or a C₁-C₆-alkyl- group;
or
N(R⁸)R⁹ together
   represent a 3- to 10-membered heterocycloalkyl- group;
   wherein said 3- to 10-membered heterocycloalkyl- group is optionally substituted one, two or three times, identically or differently, with a group selected from: halo-, —OH, —N(R⁷)R⁸, C₁-C₃-alkyl-;
R¹⁰ represents a —(CH₂)ₘ—(C₃-C₇-cycloalkyl), C₁-C₆-alkyl- or a C₁-C₆-alkoxy- group;
R¹¹ represents a group selected from:
   C₁-C₅-alkyl-, —(CH₂)ₙ—(C₃-C₇-cycloalkyl), —(CH₂)ₙ— (3- to 10-membered heterocycloalkyl);
   wherein said C₁-C₅-alkyl- group is optionally substituted one, two or three times, identically or differently, with a halogen atom or a group selected from: cyano, —N(R⁸)R⁹, —N(R⁸)C(═O)R¹⁰;
   wherein said C₃-C₇-cycloalkyl- group and 3- to 10-membered heterocycloalkyl- group are optionally substituted, one time, with a group selected from: cyano, —N(R⁸)R⁹, —C(═O)—O—R⁹;
m represent an integer of 0, 1 or 2;
n represent an integer of 0, 1 or 2;
p represents an integer of 2 or 3; and
q represents an integer of 0, 1, 2 or 3;
or a tautomer, an N-oxide, a hydrate, a solvate, or a salt thereof, or a mixture of same.

The present invention further relates to methods of preparing compounds of general formula (I), to pharmaceutical compositions and combinations comprising said compounds, to the use of said compounds for manufacturing a pharmaceutical composition for the treatment or prophylaxis of a disease, as well as to intermediate compounds useful in the preparation of said compounds.

### DETAILED DESCRIPTION OF THE INVENTION

The terms as mentioned in the present text have preferably the following meanings:

The term “halogen atom”, “halo-” or “Hal-” is to be understood as meaning a fluorine, chlorine, bromine or iodine atom, preferably a fluorine or a chlorine atom.

The term “C₁-C₆-alkyl” is to be understood as preferably meaning a linear or branched, saturated, monovalent hydrocarbon group having 1, 2, 3, 4, 5 or 6 carbon atoms, e.g. a methyl, ethyl, propyl, butyl, pentyl, hexyl, iso-propyl, iso-butyl, sec-butyl, tert-butyl, iso-pentyl, 2-methylbutyl, 1-methylbutyl, 1-ethylpropyl, 1,2-dimethylpropyl, neo-pentyl, 1,1-dimethylpropyl, 4-methylpentyl, 3-methylpentyl, 2-methylpentyl, 1-methylpentyl, 2-ethylbutyl, 1-ethylbutyl, 3, 3-dimethylbutyl, 2,2-dimethylbutyl, 1,1-dimethylbutyl, 2,3-dimethylbutyl, 1,3-dimethylbutyl, or 1,2-dimethylbutyl group, or an isomer thereof. Particularly, said group has 1, 2, 3 or 4 carbon atoms (“C₁-C₄-alkyl”), e.g. a methyl, ethyl, propyl, butyl, iso-propyl, iso-butyl, sec-butyl, tert-butyl group, more particularly 1, 2 or 3 carbon atoms (“C₁-C₃-alkyl”), e.g. a methyl, ethyl, n-propyl- or iso-propyl group.

The term “halo-C₁-C₆-alkyl” is to be understood as preferably meaning a linear or branched, saturated, monovalent hydrocarbon group in which the term “C₁-C₆-alkyl” is defined supra, and in which one or more hydrogen atoms is replaced by a halogen atom, in identically or differently, i.e. one halogen atom being independent from another. Particularly, said halogen atom is F. Said halo-C₁-C₆-alkyl group is, for example, —CF₃, —CHF₂, —CH₂F, —CF₂CF₃, or —CH₂CF₃.

The term “C₁-C₆-alkoxy” is to be understood as preferably meaning a linear or branched, saturated, monovalent, hydrocarbon group of formula —O—(C₁-C₆-alkyl), in which the term “C₁-C₆-alkyl” is defined supra, e.g. a methoxy, ethoxy, n-propoxy, iso-propoxy, n-butoxy, iso-butoxy, tert-butoxy, sec-butoxy, pentoxy, iso-pentoxy, or n-hexoxy group, or an isomer thereof.

The term “halo-C₁-C₆-alkoxy” is to be understood as preferably meaning a linear or branched, saturated, monovalent C₁-C₆-alkoxy group, as defined supra, in which one or more of the hydrogen atoms is replaced, in identically or differently, by a halogen atom. Particularly, said halogen atom is F. Said halo-C₁-C₆-alkoxy group is, for example, —OCF₃, —OCHF₂, —OCH₂F, —OCF₂CF₃, or —OCH₂CF₃.

The term “C₁-C₆-alkoxy-C₁-C₆-alkyl” is to be understood as preferably meaning a linear or branched, saturated, monovalent C₁-C₆-alkyl group, as defined supra, in which one or more of the hydrogen atoms is replaced, in identically or differently, by a C₁-C₆-alkoxy group, as defined supra, e.g. methoxyalkyl, ethoxyalkyl, propyloxyalkyl, iso-propoxyalkyl, butoxyalkyl, iso-butoxyalkyl, tert-butoxyalkyl, sec-butoxyalkyl, pentyloxyalkyl, iso-pentyloxyalkyl, hexyloxyalkyl group, or an isomer thereof.

The term “halo-C₁-C₆-alkoxy-C₁-C₆-alkyl” is to be understood as preferably meaning a linear or branched, saturated, monovalent C₁-C₆-alkoxy-C₁-C₆-alkyl group, as defined supra, in which one or more of the hydrogen atoms is replaced, in identically or differently, by a halogen atom. Particularly, said halogen atom is F. Said halo-C₁-C₆-alkoxy-C₁-C₆-alkyl group is, for example, —CH₂CH₂OCF₃, —CH₂CH₂OCHF₂, —CH₂CH₂OCH₂F, —CH₂CH₂OCF₂CF₃, or —CH₂CH₂OCH₂CF₃.

The term “C₂-C₆-alkenyl” is to be understood as preferably meaning a linear or branched, monovalent hydrocarbon group, which contains one or more double bonds, and which has 2, 3, 4, 5 or 6 carbon atoms, particularly 3 or 4 carbon atoms (“C₃-C₄-alkenyl”), it being understood that in the case in which said alkenyl group contains more than one double bond, then said double bonds may be isolated from, or conjugated with, each other. Said alkenyl group is, for example, a vinyl, allyl, (E)-2-methylvinyl, (Z)-2-methylvinyl, homoallyl, (E)-but-2-enyl, (Z)-but-2-enyl, (E)-but-1-enyl, (Z)-but-1-enyl, pent-4-enyl, (E)-pent-3-enyl, (Z)-pent-3-enyl, (E)-pent-2-enyl, (Z)-pent-2-enyl, (E)-pent-1-enyl, (Z)-pent-1-enyl, hex-5-enyl, (E)-hex-4-enyl, (Z)-hex-4-enyl, (E)-hex-3-enyl, (Z)-hex-3-enyl, (E)-hex-2-enyl, (Z)-hex-2-enyl, (E)-hex-1-enyl, (Z)-hex-1-enyl, iso-propenyl, 2-methylprop-2-enyl, 1-methylprop-2-enyl, 2-methylprop-1-enyl, (E)-1-methylprop-1-enyl, (Z)-1-methylprop-1-enyl, 3-methylbut-3-enyl, 2-methylbut-3-enyl, 1-methylbut-3-enyl, 3-methylbut-2-enyl, (E)-2-methylbut-2-enyl, (Z)-2-methylbut-2-enyl, (E)-1-methylbut-2-enyl, (Z)-1-methylbut-2-enyl, (E)-3-methylbut-1-enyl, (Z)-3-methylbut-1-enyl, (E)-2-methylbut-1-enyl, (Z)-2-methylbut-1-enyl, (E)-1-methylbut-1-enyl, (Z)-1-methylbut-1-enyl, 1, 1-dimethylprop-2-enyl, 1-ethylprop-1-enyl, 1-propylvinyl, 1-isopropylvinyl, 4-methylpent-4-enyl, 3-methylpent-4-enyl, 2-methylpent-4-enyl, 1-methylpent-4-enyl, 4-methylpent-3-enyl, (E)-3-methylpent-3-enyl, (Z)-3-methylpent-3-enyl, (E)-2-methylpent-3-enyl, (Z)-2-methylpent-3-enyl, (E)-1-methylpent-3-enyl, (Z)-1-methylpent-3-enyl, (E)-4-methylpent-2-enyl, (Z)-4-methylpent-2-enyl, (E)-3-methylpent-2-enyl, (Z)-3-methylpent-2-enyl, (E)-2-methylpent-2-enyl, (Z)-2-methylpent-2-enyl, (E)-1-methylpent-2-enyl, (Z)-1-methylpent-2-enyl, (E)-4-methylpent-1-enyl, (Z)-4-methylpent-1-enyl, (E)-3-methylpent-1-enyl, (Z)-3-methylpent-1-enyl, (E)-2-methylpent-1-enyl, (Z)-2-methylpent-1-enyl, (E)-1-methylpent-1-enyl, (Z)-1-methylpent-1-enyl, 3-ethylbut-3-enyl, 2-ethylbut-3-enyl, 1-ethylbut-3-enyl, (E)-3-ethylbut-2-enyl, (Z)-3-ethylbut-2-enyl, (E)-2-ethylbut-2-enyl, (Z)-2-ethylbut-2-enyl, (E)-1-ethylbut-2-enyl, (Z)-1-ethylbut-2-enyl, (E)-3-ethylbut-1-enyl, (Z)-3-ethylbut-1-enyl, 2-ethylbut-1-enyl, (E)-1-ethylbut-1-enyl, (Z)-1-ethylbut-1-enyl, 2-propylprop-2-enyl, 1-propylprop-2-enyl, 2-isopropylprop-2-enyl, 1-isopropylprop-2-enyl, (E)-2-propylprop-1-enyl, (Z)-2-propylprop-1-enyl, (E)-1-propylprop-1-enyl, (Z)-1-propylprop-1-enyl, (E)-2-isopropylprop-1-enyl, (Z)-2-isopropylprop-1-enyl, (E)-1-isopropylprop-1-enyl, (Z)-1-isopropylprop-1-enyl, (E)-3,3-dimethylprop-1-enyl, (Z)-3,3-dimethylprop-1-enyl, 1-(1,1-dimethylethyl)ethenyl, buta-1,3-dienyl, penta-1,4-dienyl, hexa-1,5-dienyl, or methylhexadienyl group. Particularly, said group is allyl.

The term “C₂-C₆-alkynyl” is to be understood as preferably meaning a linear or branched, monovalent hydrocarbon group which contains one or more triple bonds, and which contains 2, 3, 4, 5 or 6 carbon atoms, particularly 3 or 4 carbon atoms (“C₃-C₄-alkynyl”). Said C₂-C₆-alkynyl group is, for example, ethynyl, prop-1-ynyl, prop-2-ynyl, but-1-ynyl, but-2-ynyl, but-3-ynyl, pent-1-ynyl, pent-2-ynyl, pent-3-ynyl, pent-4-ynyl, hex-1-ynyl, hex-2-ynyl, hex-3-ynyl, hex-4-ynyl, hex-5-ynyl, 1-methylprop-2-ynyl, 2-methylbut-3-ynyl, 1-methylbut-3-ynyl, 1-methylbut-2-ynyl, 3-methylbut-1-ynyl, 1-ethylprop-2-ynyl, 3-methylpent-4-ynyl, 2-methylpent-4-ynyl, 1-methylpent-4-ynyl, 2-methylpent-3-ynyl, 1-methylpent-3-ynyl, 4-methylpent-2-ynyl, 1-methylpent-2-ynyl, 4-methylpent-1-ynyl, 3-methylpent-1-ynyl, 2-ethylbut-3-ynyl, 1-ethylbut-3-ynyl, 1-ethylbut-2-ynyl, 1-propylprop-2-ynyl, 1-isopropylprop-2-ynyl, 2, 2-dimethylbut-3-ynyl, 1,1-dimethylbut-3-ynyl, 1,1-dimethylbut-2-ynyl, or 3,3-dimethylbut-1-ynyl group. Particularly, said alkynyl group is prop-1-ynyl or prop-2-ynyl.

The term “C₃-C₇-cycloalkyl” is to be understood as meaning a saturated, monovalent, monocyclic hydrocarbon ring which contains 3, 4, 5, 6 or 7 carbon atoms. Said C₃-C₇-cycloalkyl group is for example a cyclopropyl, cyclobutyl, cyclopentyl, cyclohexyl or cycloheptyl ring. Particularly, said ring contains 3, 4, 5 or 6 carbon atoms (“C₃-C₆-cycloalkyl”).

The term “C₄-C₇-cycloalkenyl” is to be understood as preferably meaning a monovalent, monocyclic hydrocarbon ring which contains 4, 5, 6 or 7 carbon atoms and one or two double bonds, in conjugation or not, as the size of said cycloalkenyl ring allows. Said C₄-C₇-cycloalkenyl group is for example a cyclobutenyl, cyclopentenyl, or cyclohexenyl group.

The term “3- to 10-membered heterocycloalkyl” is to be understood as meaning a saturated, monovalent, mono- or bicyclic hydrocarbon ring which contains 2, 3, 4, 5, 6, 7, 8 or 9 carbon atoms, and one or more heteroatom-containing groups selected from C(═O), O, S, S(═O), S(═O)₂, NR^{a}, in which R^{a} represents a hydrogen atom or a C₁-C₆-alkyl- or C₃-C₇-cycloalkyl- group; it being possible for said heterocycloalkyl group to be attached to the rest of the molecule via any one of the carbon atoms or, if present, the nitrogen atom. Heterospirocycloalkyl, heterobicycloalkyl and bridged heterocycloalkyl, as defined infra, are also included within the scope of this definition.

The term “heterospirocycloalkyl” is to be understood as meaning a saturated, monovalent bicyclic hydrocarbon radical in which the two rings share one common ring carbon atom, and wherein said bicyclic hydrocarbon radical contains 2, 3, 4, 5, 6, 7, 8 or 9 carbon atoms, and one or more heteroatom-containing groups selected from C(═O), O, S, S(═O), S(═O)₂, NR^{a}, in which R^{a} represents a hydrogen atom or a C₁-C₆-alkyl- or C₃-C₇-cycloalkyl- group; it being possible for said heterospirocycloalkyl- group to be attached to the rest of the molecule via any one of the carbon atoms or, if present, the nitrogen atom. Said heterospirocycloalkyl- group is, for example, azaspiro[2.3]hexyl-, azaspiro[3.3]heptyl-, oxaazaspiro[3.3]heptyl-, thiaazaspiro[3.3]heptyl-, oxaspiro[3.3]heptyl-, oxazaspiro[5.3]nonyl-, oxazaspiro[4.3]octyl-, oxazaspiro[5.5]undecyl-, diazaspiro[3.3]heptyl-, thiazaspiro[3.3]heptyl-, thiazaspiro[4.3]octyl-, or azaspiro[5.5]decyl-.

The term “heterobicycloalkyl” is to be understood as meaning a saturated, monovalent bicyclic hydrocarbon radical in which the two rings share two immediately adjacent ring atoms, and wherein said bicyclic hydrocarbon radical contains 2, 3, 4, 5, 6, 7, 8 or 9 carbon atoms, and one or more heteroatom-containing groups selected from C(═O), O, S, S(═O), S(═O)₂, NR^{a}, in which R^{a} represents a hydrogen atom or a C₁-C₆-alkyl- or C₃-C₇-cycloalkyl- group; it being possible for said heterobicycloalkyl- group to be attached to the rest of the molecule via any one of the carbon atoms or, if present, the nitrogen atom. Said heterobicycoalkyl- group is, for example, azabicyclo[3.3.0]octyl-, azabicyclo[4.3.0]nonyl-, diazabicyclo[4.3.0]nonyl-, oxazabicyclo[4.3.0]nonyl-, thiazabicyclo[4.3.0]nonyl-, or azabicyclo[4.4.0]decyl-.

The term “bridged heterocycloalkyl” is to be understood as meaning a saturated, monovalent bicyclic hydrocarbon radical in which the two rings share two common ring atoms which are not immediately adjacent, and wherein said bicyclic hydrocarbon radical contains 2, 3, 4, 5, 6, 7, 8 or 9 carbon atoms, and one or more heteroatom-containing groups selected from C(═O), O, S, S(═O), S(═O)₂, NR^{a}, in which R^{a} represents a hydrogen atom, or a C₁-C₆-alkyl- or C₃-C₇-cycloalkyl- group; it being possible for said bridged heterocycloalkyl- group to be attached to the rest of the molecule via any one of the carbon atoms or, if present, the nitrogen atom. Said bridged heterocycloalkyl- group is, for example, azabicyclo[2.2.1]heptyl-, oxazabicyclo[2.2.1]heptyl-, thiazabicyclo[2.2.1]heptyl-, diazabicyclo[2.2.1]heptyl-, azabicyclo[2.2.2]octyl-, diazabicyclo[2.2.2]octyl-, oxazabicyclo[2.2.2]octyl-, thiazabicyclo[2.2.2]octyl-, azabicyclo[3.2.1]octyl-, diazabicyclo[3.2.1]octyl-, oxazabicyclo[3.2.1]octyl-, thiazabicyclo[3.2.1]octyl-, azabicyclo[3.3.1]nonyl-, diazabicyclo[3.3.1]nonyl-, oxazabicyclo[3.3.1]nonyl-, thiazabicyclo[3.3.1]nonyl-, azabicyclo[4.2.1]nonyl-, diazabicyclo[4.2.1]nonyl-, oxazabicyclo[4.2.1]nonyl, thiazabicyclo[4.2.1]nonyl-, azabicyclo[3.3.2]decyl-, diazabicyclo[3.3.2]decyl-, oxazabicyclo[3.3.2]decyl-, thiazabicyclo[3.3.2]decyl-, or azabicyclo[4.2.2]decyl-.

Particularly, said 3- to 10-membered heterocycloalkyl can contain 2, 3, 4, or 5 carbon atoms, and one or more of the above-mentioned heteroatom-containing groups (a “3- to 6-membered heterocycloalkyl”), more particularly said heterocycloalkyl can contain 4 or 5 carbon atoms, and one or more of the above-mentioned heteroatom-containing groups (a “5- to 6-membered heterocycloalkyl”).

Particularly, without being limited thereto, said heterocycloalkyl can be a 4-membered ring, such as an azetidinyl, oxetanyl, or a 5-membered ring, such as tetrahydrofuranyl, dioxolinyl, pyrrolidinyl, imidazolidinyl, pyrazolidinyl, or a 6-membered ring, such as tetrahydropyranyl, piperidinyl, morpholinyl, dithianyl, thiomorpholinyl, piperazinyl, or trithianyl, or a 7-membered ring, such as a diazepanyl ring, for example.

The term “4- to 10-membered heterocycloalkenyl”, is to be understood as meaning an unsaturated, monovalent, mono- or bicyclic hydrocarbon ring which contains 3, 4, 5, 6, 7, 8 or 9 carbon atoms, and one or more heteroatom-containing groups selected from C(═O), O, S, S(═O), S(═O)₂, NR^{a}, in which R^{a} represents a hydrogen atom or a C₁-C₆-alkyl- group; it being possible for said heterocycloalkenyl group to be attached to the rest of the molecule via any one of the carbon atoms or, if present, the nitrogen atom. Examples of said heterocycloalkenyl may contain one or more double bonds, e.g. 4H-pyranyl, 2H-pyranyl, 3H-diazirinyl, 2,5-dihydro-1H-pyrrolyl, [1,3]dioxolyl, 4H-[1,3,4]thiadiazinyl, 2,5-dihydrofuranyl, 2,3-dihydrofuranyl, 2,5-dihydrothiophenyl, 2,3-dihydrothiophenyl, 4,5-dihydrooxazolyl, or 4H-[1,4]thiazinyl group.

The term “aryl” is to be understood as preferably meaning a monovalent, aromatic or partially aromatic, mono-, or bi- or tricyclic hydrocarbon ring having 6, 7, 8, 9, 10, 11, 12, 13 or 14 carbon atoms (a “C₆-C₁₄-aryl” group), particularly a ring having 6 carbon atoms (a “C₆-aryl” group), e.g. a phenyl group; or a ring having 9 carbon atoms (a “C₉-aryl” group), e.g. an indanyl or indenyl group, or a ring having 10 carbon atoms (a “C₁₀-aryl” group), e.g. a tetralinyl, dihydronaphthyl, or naphthyl group, or a biphenyl group (a “C₁₂-aryl” group), or a ring having 13 carbon atoms, (a “C₁₃-aryl” group), e.g. a fluorenyl group, or a ring having 14 carbon atoms, (a “C₁₄-aryl” group), e.g. an anthracenyl group. Preferably, the aryl group is a phenyl group.

The term “heteroaryl” is understood as preferably meaning a monovalent, monocyclic-, bicyclic- or tricyclic aromatic ring system having 5, 6, 7, 8, 9, 10, 11, 12, 13 or 14 ring atoms (a “5- to 14-membered heteroaryl” group), particularly 5 or 6 or 9 or 10 atoms, and which contains at least one heteroatom which may be identical or different, said heteroatom being such as oxygen, nitrogen or sulfur, and in addition in each case can be benzocondensed. Particularly, heteroaryl is selected from thienyl, furanyl, pyrrolyl, oxazolyl, thiazolyl, imidazolyl, pyrazolyl, isoxazolyl, isothiazolyl, oxadiazolyl, triazolyl, thiadiazolyl, thia-4H-pyrazolyl etc., and benzo derivatives thereof, such as, for example, benzofuranyl, benzothienyl, benzoxazolyl, benzisoxazolyl, benzimidazolyl, benzotriazolyl, indazolyl, indolyl, isoindolyl, etc.; or pyridinyl, pyridazinyl, pyrimidinyl, pyrazinyl, triazinyl, etc., and benzo derivatives thereof, such as, for example, quinolinyl, quinazolinyl, isoquinolinyl, etc.; or azocinyl, indolizinyl, purinyl, etc., and benzo derivatives thereof; or cinnolinyl, phthalazinyl, quinazolinyl, quinoxalinyl, naphthpyridinyl, pteridinyl, carbazolyl, acridinyl, phenazinyl, phenothiazinyl, phenoxazinyl, xanthenyl, or oxepinyl, etc.

In general, and unless otherwise mentioned, the heteroarylic or heteroarylenic radicals include all the possible isomeric forms thereof, e.g. the positional isomers thereof. Thus, for some illustrative non-restricting example, the term pyridyl includes pyridin-2-yl, pyridin-3-yl, and pyridin-4-yl; or the term thienyl includes thien-2-yl and thien-3-yl. Preferably, the heteroaryl group is a pyridinyl group.

The term “C₁-C₆”, as used throughout this text, e.g. in the context of the definition of “C₁-C₆-alkyl”, “C₁-C₆-haloalkyl”, “C₁-C₆-alkoxy”, or “C₁-C₆-haloalkoxy” is to be understood as meaning an alkyl group having a finite number of carbon atoms of 1 to 6, i.e. 1, 2, 3, 4, 5, or 6 carbon atoms. It is to be understood further that said term “C₁-C₆” is to be interpreted as any sub-range comprised therein, e.g. C₁-C₆, C₂-C₅, C₃-C₄, C₁-C₂, C₁-C₃, C₁-C₄, C₁-C₅; particularly C₁-C₂, C₁-C₃, C₁-C₄, C₁-C₅, C₁-C₆; more particularly C₁-C₄; in the case of “C₁-C₆-haloalkyl” or “C₁-C₆-haloalkoxy” even more particularly C₁-C₂.

Similarly, as used herein, the term “C₂-C₆”, as used throughout this text, e.g. in the context of the definitions of “C₂-C₆-alkenyl” and “C₂-C₆-alkynyl”, is to be understood as meaning an alkenyl group or an alkynyl group having a finite number of carbon atoms of 2 to 6, i.e. 2, 3, 4, 5, or 6 carbon atoms. It is to be understood further that said term “C₂-C₆” is to be interpreted as any sub-range comprised therein, e.g. C₂-C₆, C₃-C₅, C₃-C₄, C₂-C₃, C₂-C₄, C₂-C₅; particularly C₂-C₃.

Further, as used herein, the term “C₃-C₇”, as used throughout this text, e.g. in the context of the definition of “C₃-C₇-cycloalkyl”, is to be understood as meaning a cycloalkyl group having a finite number of carbon atoms of 3 to 7, i.e. 3, 4, 5, 6 or 7 carbon atoms. It is to be understood further that said term “C₃-C₇” is to be interpreted as any sub-range comprised therein, e.g. C₃-C₆, C₄-C₅, C₃-C₅, C₃-C₄, C₄-C₆, C₅-C₇; particularly C₃-C₆.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds.

The term “optionally substituted” means that the number of substituents can be zero. Unless otherwise indicated, optionally substituted groups may be substituted with as many optional substituents as can be accommodated by replacing a hydrogen atom with a non-hydrogen substituent on any available carbon or nitrogen atom. Commonly, the number of optional substituents (when present) ranges from 1 to 3.

Ring system substituent means a substituent attached to an aromatic or nonaromatic ring system which, for example, replaces an available hydrogen on the ring system.

As used herein, the term “one or more”, e.g. in the definition of the substituents of the compounds of the general formulae of the present invention, is understood as meaning “one, two, three, four or five, particularly one, two, three or four, more particularly one, two or three, even more particularly one or two”.

The invention also includes all suitable isotopic variations of a compound of the invention. An isotopic variation of a compound of the invention is defined as one in which at least one atom is replaced by an atom having the same atomic number but an atomic mass different from the atomic mass usually or predominantly found in nature. Examples of isotopes that can be incorporated into a compound of the invention include isotopes of hydrogen, carbon, nitrogen, oxygen, phosphorus, sulphur, fluorine, chlorine, bromine and iodine, such as ²H (deuterium), ³H (tritium), ¹¹C, ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁹I and ¹³¹I, respectively. Certain isotopic variations of a compound of the invention, for example, those in which one or more radioactive isotopes such as ³H or ¹⁴C are incorporated, are useful in drug and/or substrate tissue distribution studies. Tritiated and carbon-14, i.e., ¹⁴C, isotopes are particularly preferred for their ease of preparation and detectability. Further, substitution with isotopes such as deuterium may afford certain therapeutic advantages resulting from greater metabolic stability, for example, increased in vivo half-life or reduced dosage requirements and hence may be preferred in some circumstances. Isotopic variations of a compound of the invention can generally be prepared by conventional procedures known by a person skilled in the art such as by the illustrative methods or by the preparations described in the examples hereafter using appropriate isotopic variations of suitable reagents.

Optical isomers can be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, ditoluoyltartaric and camphorsulfonic acid. Mixtures of diastereoisomers can be separated into their individual diastereomers on the basis of their physical and/or chemical differences by methods known in the art, for example, by chromatography or fractional crystallisation. The optically active bases or acids are then liberated from the separated diastereomeric salts. A different process for separation of optical isomers involves the use of chiral chromatography (e.g., chiral HPLC columns), with or without conventional derivatisation, optimally chosen to maximise the separation of the enantiomers. Suitable chiral HPLC columns are manufactured by Daicel, e.g., Chiracel OD and Chiracel OJ among many others, all routinely selectable. Enzymatic separations, with or without derivatisation, are also useful. The optically active compounds of this invention can likewise be obtained by chiral syntheses utilizing optically active starting materials.

In order to limit different types of isomers from each other reference is made to IUPAC Rules Section E (Pure Appl Chem 45, 11-30, 1976).

The present invention includes all possible double bond isomers and in case a second stereogenic centre is present, diastereomers of the compounds of the present invention as single stereoisomers, or as any mixture of said stereoisomers, e.g. (E)- or (Z)-isomers, in any ratio. Isolation of a single stereoisomer, e.g. a single single diastereomer, of a compound of the present invention may be achieved by any suitable state of the art method, such as chromatography, especially chiral chromatography, for example.

Further, the compounds of the present invention may exist as tautomers. For example, any compound of the present invention which contains a pyrazole moiety as a heteroaryl group for example can exist as a 1H tautomer, or a 2H tautomer, or even a mixture in any amount of the two tautomers, or a triazole moiety for example can exist as a 1H tautomer, a 2H tautomer, or a 4H tautomer, or even a mixture in any amount of said 1H, 2H and 4H tautomers, namely:

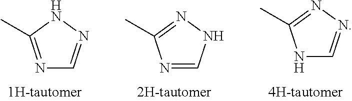

The present invention includes all possible tautomers of the compounds of the present invention as single tautomers, or as any mixture of said tautomers, in any ratio.

Further, the compounds of the present invention can exist as N-oxides, which are defined in that at least one nitrogen of the compounds of the present invention is oxidised. The present invention includes all such possible N-oxides.

The present invention also relates to useful forms of the compounds as disclosed herein, such as metabolites, hydrates, solvates, prodrugs, salts, in particular pharmaceutically acceptable salts, and co-precipitates.

Where the plural form of the word compounds, salts, polymorphs, hydrates, solvates and the like, is used herein, this is taken to mean also a single compound, salt, polymorph, isomer, hydrate, solvate or the like.

By “stable compound’ or “stable structure” is meant a compound that is sufficiently robust to survive isolation to a useful degree of purity from a reaction mixture, and formulation into an efficacious therapeutic agent.

The compounds of the present invention can exist as a hydrate, or as a solvate, wherein the compounds of the present invention contain polar solvents, in particular water, methanol or ethanol for example as structural element of the crystal lattice of the compounds. The amount of polar solvents, in particular water, may exist in a stoichiometric or non-stoichiometric ratio. In the case of stoichiometric solvates, e.g. a hydrate, hemi-, (semi-), mono-, sesqui-, di-, tri-, tetra-, penta- etc. solvates or hydrates, respectively, are possible. The present invention includes all such hydrates or solvates.

Further, the compounds of the present invention can exist in free form, e.g. as a free base, or as a free acid, or as a zwitterion, or can exist in the form of a salt. Said salt may be any salt, either an organic or inorganic addition salt, particularly any pharmaceutically acceptable organic or inorganic addition salt, customarily used in pharmacy.

The term “pharmaceutically acceptable salt” refers to a relatively non-toxic, inorganic or organic acid addition salt of a compound of the present invention. For example, see S. M. Berge, et al. “Pharmaceutical Salts,” J. Pharm. Sci. 1977, 66, 1-19. A suitable pharmaceutically acceptable salt of the compounds of the present invention may be, for example, an acid-addition salt of a compound of the present invention bearing a nitrogen atom, in a chain or in a ring, for example, which is sufficiently basic, such as an acid-addition salt with an inorganic acid, such as hydrochloric, hydrobromic, hydroiodic, sulfuric, bisulfuric, phosphoric, or nitric acid, for example, or with an organic acid, such as formic, acetic, acetoacetic, pyruvic, trifluoroacetic, propionic, butyric, hexanoic, heptanoic, undecanoic, lauric, benzoic, salicylic, 2-(4-hydroxybenzoyl)-benzoic, camphoric, cinnamic, cyclopentanepropionic, digluconic, 3-hydroxy-2-naphthoic, nicotinic, pamoic, pectinic, persulfuric, 3-phenylpropionic, picric, pivalic, 2-hydroxyethanesulfonate, itaconic, sulfamic, trifluoromethanesulfonic, dodecylsulfuric, ethansulfonic, benzenesulfonic, para-toluenesulfonic, methansulfonic, 2-naphthalenesulfonic, naphthalinedisulfonic, camphorsulfonic acid, citric, tartaric, stearic, lactic, oxalic, malonic, succinic, malic, adipic, alginic, maleic, fumaric, D-gluconic, mandelic, ascorbic, glucoheptanoic, glycerophosphoric, aspartic, sulfosalicylic, hemisulfuric, or thiocyanic acid, for example.

Further, another suitably pharmaceutically acceptable salt of a compound of the present invention which is sufficiently acidic, is an alkali metal salt, for example a sodium or potassium salt, an alkaline earth metal salt, for example a calcium or magnesium salt, an ammonium salt or a salt with an organic base which affords a physiologically acceptable cation, for example a salt with N-methyl-glucamine, dimethyl-glucamine, ethyl-glucamine, lysine, dicyclohexylamine, 1,6-hexadiamine, ethanolamine, glucosamine, sarcosine, serinol, tris-hydroxy-methyl-aminomethane, aminopropandiol, sovak-base, 1-amino-2,3,4-butantriol. Additionally, basic nitrogen containing groups may be quaternised with such agents as lower alkyl halides such as methyl, ethyl, propyl, and butyl chlorides, bromides and iodides; dialkyl sulfates like dimethyl, diethyl, and dibutyl sulfate; and diamyl sulfates, long chain halides such as decyl, lauryl, myristyl and stearyl chlorides, bromides and iodides, aralkyl halides like benzyl and phenethyl bromides and others.

Those skilled in the art will further recognise that acid addition salts of the claimed compounds may be prepared by reaction of the compounds with the appropriate inorganic or organic acid via any of a number of known methods. Alternatively, alkali and alkaline earth metal salts of acidic compounds of the invention are prepared by reacting the compounds of the invention with the appropriate base via a variety of known methods.

The present invention includes all possible salts of the compounds of the present invention as single salts, or as any mixture of said salts, in any ratio.

As used herein, the term “in vivo hydrolysable ester” is understood as meaning an in vivo hydrolysable ester of a compound of the present invention containing a carboxy or hydroxy group, for example, a pharmaceutically acceptable ester which is hydrolysed in the human or animal body to produce the parent acid or alcohol. Suitable pharmaceutically acceptable esters for carboxy include for example alkyl, cycloalkyl and optionally substituted phenylalkyl, in particular benzyl esters, C₁-C₆ alkoxymethyl esters, e.g. methoxymethyl, C₁-C₆ alkanoyloxymethyl esters, e.g. pivaloyloxymethyl, phthalidyl esters, C₃-C₈ cycloalkoxy-carbonyloxy-C₁-C₆ alkyl esters, e.g. 1-cyclohexylcarbonyloxyethyl; 1,3-dioxolen-2-onylmethyl esters, e.g. 5-methyl-1,3-dioxolen-2-onylmethyl; and C₁-C₆-alkoxycarbonyloxyethyl esters, e.g. 1-methoxycarbonyloxyethyl, and may be formed at any carboxy group in the compounds of this invention. An in vivo hydrolysable ester of a compound of the present invention containing a hydroxy group includes inorganic esters such as phosphate esters and [alpha]-acyloxyalkyl ethers and related compounds which as a result of the in vivo hydrolysis of the ester breakdown to give the parent hydroxy group. Examples of [alpha]-acyloxyalkyl ethers include acetoxymethoxy and 2,2-dimethylpropionyloxymethoxy. A selection of in vivo hydrolysable ester forming groups for hydroxy include alkanoyl, benzoyl, phenylacetyl and substituted benzoyl and phenylacetyl, alkoxycarbonyl (to give alkyl carbonate esters), dialkylcarbamoyl and N-(dialkylaminoethyl)-N-alkylcarbamoyl (to give carbamates), dialkylaminoacetyl and carboxyacetyl. The present invention covers all such esters.

Furthermore, the present invention includes all possible crystalline forms, or polymorphs, of the compounds of the present invention, either as single polymorphs, or as a mixture of more than one polymorphs, in any ratio.

In accordance with a first aspect, the present invention covers compounds of general formula (I):

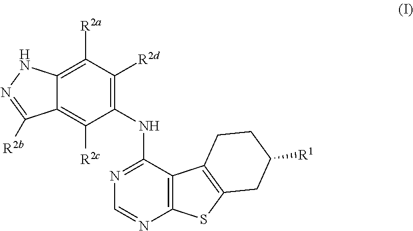

in which:
R¹ represents —C(═O)N(R³)R⁴;
R^{2a} represents a hydrogen atom;
R^{2b} represents a hydrogen atom;
R^{2c} represents a hydrogen atom or a halogen atom;
R^{2d} represents a hydrogen atom, a halogen atom, or a group selected from: cyano-, —OR⁵, —SR⁶, —S(═O)₂R⁶, —S(═O)(═NH)R⁶, —N(H)R⁷, —N(R⁶)R⁷, —N(R⁶)R¹¹;
R³ represents a group selected from:
   C₁-C₆-alkyl-, C₁-C₆-alkoxy-, C₃-C₆-alkenyl-, C₃-C₆-alkynyl-, —(CH₂)_{q}—(C₃-C₇-cycloalkyl), —(CH₂)ₚ—O—(C₃-C₇-cycloalkyl), —(CH₂)_{q}—(C₄-C₇-cycloalkenyl), —(CH₂)ₚ—O—(C₄-C₇-cycloalkenyl), —(CH₂)_{q}— (3- to 10-membered heterocycloalkyl), —(CH₂)ₚ—O— (3- to 10-membered heterocycloalkyl), —(CH₂)_{q}— (4- to 10-membered heterocycloalkenyl), —(CH₂)ₚ—O— (4- to 10-membered heterocycloalkenyl), —(CH₂)_{q}-aryl, —(CH₂)ₚ—O-aryl, —(CH₂)_{q}-heteroaryl, —(CH₂)ₚ—O-heteroaryl, —S(═O)₂—R⁶;
   wherein said C₁-C₆-alkyl- group is optionally substituted one, two or three times, identically or differently, with a group selected from:
   halo-, C₁-C₃-alkoxy-, HO—, —N(R⁸)R⁹;
R⁴ represents a C₁-C₄-alkyl- group;
   wherein said C₁-C₄-alkyl- group is optionally substituted one, two or three times, identically or differently, with a halogen atom or a group selected from: HO—, C₁-C₃-alkoxy-, —CN, —N(R⁸)R⁹, —N(R⁷)R⁸, —C(═O)N(R⁸)R⁹, —C(═O)N(R⁷)R⁸;
or
N(R³)R⁴ together
   represent a 3- to 10-membered heterocycloalkyl- or 4- to 10-membered heterocycloalkenyl group;
   wherein said 3- to 10-membered heterocycloalkyl- group is optionally substituted one, two or three times, identically or differently, with a halogen atom or a group selected from: —(CH₂)_{q}—OH, —N(R⁷)R⁸, —N(R⁸)R⁹, C₁-C₃-alkyl-, —CN, —C(═O)R¹⁰, —C(═O)N(R⁸)R⁹, —(CH₂)_{q}-aryl, —(CH₂)_{q}-heteroaryl, —(C₁-C₃-alkyl)-N(R⁸)R⁹;
R⁵ represents a hydrogen atom or a group selected from:
   C₁-C₅-alkyl-, —(CH₂)ₘ—(C₃-C₇-cycloalkyl), —(CH₂)ₘ— (3- to 10-membered heterocycloalkyl);
   wherein said C₁-C₅-alkyl- group is optionally substituted one, two or three times, identically or differently, with a halogen atom or a group selected from: cyano, —N(R⁸)R⁹, —N(R⁸)C(═O)R¹⁰, -azido, phenyl-;
   wherein said C₃-C₇-cycloalkyl- group and said 3- to 10-membered heterocycloalkyl- group are optionally substituted, one time, with a group selected from: cyano, —N(R⁸)R⁹, —C(═O)—O—R⁹;
R⁶ represents a hydrogen atom or a C₁-C₄-alkyl- group;
R⁷ represents a C₁-C₄-alkyl-, C₃-C₄-alkenyl- or C₁-C₃-alkoxy- group; wherein said C₁-C₄-alkyl- is optionally substituted once with —OH or —N(R⁸)R⁹;
or
N(R⁶)R⁷ together
   represent a 3- to 10-membered heterocycloalkyl- or 4- to 10-membered heterocycloalkenyl group;
   wherein said 3- to 10-membered heterocycloalkyl- group is optionally substituted once with —N(R⁸)R⁹;
R⁸ represents a hydrogen atom or a C₁-C₄-alkyl- group;
R⁹ represents a hydrogen atom or a C₁-C₆-alkyl- group;
or
N(R⁸)R⁹ together
   represent a 3- to 10-membered heterocycloalkyl- group;
   wherein said 3- to 10-membered heterocycloalkyl- group is optionally substituted one, two or three times, identically or differently, with a group selected from: halo-, —OH, —N(R⁷)R⁸, C₁-C₃-alkyl-;
R¹⁰ represents a —(CH₂)ₘ—(C₃-C₇-cycloalkyl), C₁-C₆-alkyl- or a C₁-C₆-alkoxy- group;
R¹¹ represents a group selected from:
   C₁-C₅-alkyl-, —(CH₂)ₙ—(C₃-C₇-cycloalkyl), —(CH₂)ₙ— (3- to 10-membered heterocycloalkyl);
   wherein said C₁-C₅-alkyl- group is optionally substituted one, two or three times, identically or differently, with a halogen atom or a group selected from: cyano, —N(R⁸)R⁹, —N(R⁸)C(═O)R¹⁰;
   wherein said C₃-C₇-cycloalkyl- group and said 3- to 10-membered heterocycloalkyl- group are optionally substituted, one time, with a group selected from: cyano, —N(R⁸)R⁹, —C(═O)—O—R⁹;
m represent an integer of 0, 1 or 2;
n represent an integer of 0, 1 or 2;
p represents an integer of 2 or 3; and
q represents an integer of 0, 1, 2 or 3;
or a tautomer, an N-oxide, a hydrate, a solvate, or a salt thereof, or a mixture of same.

In a preferred embodiment, the invention relates to compounds of formula (I), supra, wherein R^{2c} represents a hydrogen atom or a halogen atom.

In another preferred embodiment, the invention relates to compounds of formula (I), supra, wherein R^{2c} represents a hydrogen atom or a fluoro atom.

In another preferred embodiment, the invention relates to compounds of formula (I), supra, wherein R^{2c} represents a hydrogen atom.

In another preferred embodiment, the invention relates to compounds of formula (I), supra, wherein R^{2c} represents a halogen atom.

In another preferred embodiment, the invention relates to compounds of formula (I), supra, wherein R^{2c} represents a fluoro atom.

In another preferred embodiment, the invention relates to compounds of formula (I), supra, wherein R^{2d} represents a hydrogen atom, a halogen atom, or a group selected from: cyano-, —OR⁵, —SR⁶, —S(═O)₂R⁶, —S(═O)(═NH)R⁶, —N(H)R⁷, —N(R⁶)R⁷, —N(R⁶)R¹¹.

In another preferred embodiment, the invention relates to compounds of formula (I), supra, wherein R^{2d} represents a hydrogen atom, a halogen atom, or a group selected from: cyano-, —OR⁵, —SR⁶, —S(═O)₂R⁶, —S(═O)(═NH)R⁶, —N(H)R⁷, —N(R⁶)R⁷.

In another preferred embodiment, the invention relates to compounds of formula (I), supra, wherein R^{2d} represents a hydrogen atom, a halogen atom, or a group selected from: cyano-, —OR⁵, —N(H)R⁷, —N(R⁶)R⁷; wherein —OR⁵ represents C₁-C₃-alkoxy- or halo-C₁-C₃-alkoxy-.

In another preferred embodiment, the invention relates to compounds of formula (I), supra, wherein R^{2d} represents a hydrogen atom, a halogen atom, or a group selected from: —OR⁵, —SR⁶, —S(═O)₂R⁶, —N(H)R⁷, —N(R⁶)R⁷, —N(R⁶)R¹¹.

In another preferred embodiment, the invention relates to compounds of formula (I), supra, wherein R^{2d} represents a hydrogen atom, a halogen atom, or a group selected from: —OR⁵, —SR⁶, —S(═O)₂R⁶, —N(H)R⁷, —N(R⁶)R⁷.

In another preferred embodiment, the invention relates to compounds of formula (I), supra, wherein R^{2d} represents a hydrogen atom, a halogen atom, or a group selected from: cyano-, —OR⁵, —N(H)R⁷, —N(R⁶)R⁷.

In another preferred embodiment, the invention relates to compounds of formula

(I), supra, wherein R^{2d} represents a hydrogen atom, a halogen atom, or a group selected from: —OR⁵, —SR⁶, —N(R⁶)R⁷, —N(R⁶)R¹¹.

In another preferred embodiment, the invention relates to compounds of formula (I), supra, wherein R^{2d} represents a hydrogen atom, a halogen atom, or a group selected from: —OR⁵, —SR⁶, —N(R⁶)R⁷.

In another preferred embodiment, the invention relates to compounds of formula (I), supra, wherein R^{2d} represents a group selected from: —OR⁵, —SR⁶, —S(═O)₂R⁶, —S(═O)(═NH)R⁶.

In another preferred embodiment, the invention relates to compounds of formula (I), supra, wherein R^{2d} represents a hydrogen atom.

In another preferred embodiment, the invention relates to compounds of formula (I), supra, wherein R^{2d} represents a halogen atom.

In a preferred embodiment, the invention relates to compounds of formula (I), supra, wherein R^{2d} represents a group selected from: —OR⁵, —SR⁶, —S(═O)₂R⁶, —S(═O)(═NH)R⁶; with the proviso that —OR⁵ does not represent C₁-C₃-alkoxy- or halo-C₁-C₃-alkoxy-.

In another preferred embodiment, the invention relates to compounds of formula (I), supra, wherein R^{2d} represents an —OR⁵ group; and wherein R⁵ represents a C₁-C₅-alkyl- group; wherein said C₁-C₅-alkyl- group is substituted one, two or three times, identically or differently, with a group selected from: —N(R⁸)R⁹, —N(H)C(═O)R¹⁰, 3- to 10-membered heterocycloalkyl-, -azido;

or wherein R⁵ represents a C₃-C₇-cycloalkyl-, 3- to 10-membered heterocycloalkyl-group, or an unsubstituted C₄-C₅-alkyl- or halo-C₄-C₅-alkyl group.

In another preferred embodiment, the invention relates to compounds of formula (I), supra, wherein R^{2d} represents an —OR⁵ group; and wherein R⁵ represents a C₁-C₅-alkyl- group, wherein said C₁-C₅-alkyl- is substituted once with a group selected from: —N(R⁸)R⁹, —N(H)C(═O)R¹⁰, 3- to 10-membered heterocycloalkyl-, -azido; or wherein R⁵ represents a C₃-C₇-cycloalkyl-, 3- to 10-membered heterocycloalkyl- group, or an unsubstituted C₄-C₅-alkyl- or halo-C₄-C₅-alkyl group.

In another preferred embodiment, the invention relates to compounds of formula (I), supra, wherein R^{2d} represents an —OR⁵ group; and wherein R⁵ represents a C₁-C₅-alkyl- group; wherein said C₁-C₅-alkyl- group is substituted once with a group selected from: —N(R⁸)R⁹, —N(H)C(═O)R¹⁰, 3- to 7-membered heterocycloalkyl-, -azido; or wherein R⁵ represents a C₃-C₇-cycloalkyl-, 3- to 7-membered heterocycloalkyl- or an unsubstituted C₄-C₅-alkyl- group.

In another preferred embodiment, the invention relates to compounds of formula (I), supra, wherein R^{2d} represents an —OR⁵ group; and wherein R⁵ represents a C₁-C₅-alkyl- group; wherein said C₁-C₅-alkyl- group is substituted once with a group selected from: —N(R⁸)R⁹, 3- to 7-membered heterocycloalkyl-; or wherein R⁵ represents a C₃-C₇-cycloalkyl-, 3- to 7-membered heterocycloalkyl- or an unsubstituted C₄-C₅-alkyl- group.

In another preferred embodiment, the invention relates to compounds of formula (I), supra, wherein R^{2d} represents an —OR⁵ group; and wherein R⁵ represents a C₁-C₅-alkyl- group; wherein said C₁-C₅-alkyl- group is substituted once with a —N(R⁸)R⁹ group; or wherein R⁵ represents a C₃-C₇-cycloalkyl-, 3- to 7-membered heterocycloalkyl- or an unsubstituted C₄-C₅-alkyl- group.

In another preferred embodiment, the invention relates to compounds of formula (I), supra, wherein R^{2d} represents an —OR⁵ group; and wherein R⁵ represents a C₁-C₅-alkyl- group; wherein said C₁-C₅-alkyl- group is substituted once with a 3- to 7-membered heterocycloalkyl- group; or wherein R⁵ represents a C₃-C₇-cycloalkyl-, 3- to 7-membered heterocycloalkyl- or an unsubstituted C₄-C₅-alkyl- group.

In another preferred embodiment, the invention relates to compounds of formula (I), supra, wherein R^{2d} represents an —OR⁵ group; and wherein R⁵ represents a C₄-C₅-alkyl-, C₃-C₇-cycloalkyl- or 3- to 7-membered heterocycloalkyl group.

In another preferred embodiment, the invention relates to compounds of formula (I), supra, wherein R^{2d} represents an —OR⁵ group; and wherein R⁵ represents a C₃-C₇-cycloalkyl- or 3- to 7-membered heterocycloalkyl group.

In another preferred embodiment, the invention relates to compounds of formula (I), supra, wherein R^{2d} represents a —SR⁶, —S(═O)₂R⁶ or —S(═O)(═NH)R⁶ group.

In another preferred embodiment, the invention relates to compounds of formula (I), supra, wherein R^{2d} represents a —SR⁶ or a —S(═O)₂R⁶ group.

In another preferred embodiment, the invention relates to compounds of formula (I), supra, wherein R^{2d} represents a —S(═O)₂R⁶ group.

In another preferred embodiment, the invention relates to compounds of formula (I), supra, wherein R^{2d} represents a —SR⁶ group.

In another preferred embodiment, the invention relates to compounds of formula (I), supra, wherein R^{2d} represents a group selected from: cyano-, —OR⁵, —SR⁶, —N(H)R⁷, —N(R⁶)R⁷, —N(R⁶)R¹¹.

In another preferred embodiment, the invention relates to compounds of formula (I), supra, wherein R^{2d} represents a group selected from: —OR⁵, —SR⁶, —N(H)R⁷, —N(R⁶)R⁷, —N(R⁶)R¹¹.

In another preferred embodiment, the invention relates to compounds of formula (I), supra, wherein R^{2d} represents an —OR⁵ group.

In another preferred embodiment, the invention relates to compounds of formula (I), supra, wherein R^{2d} represents an —OR⁵ group; wherein R⁵ represents a C₁-C₅-alkyl-, C₃-C₇-cycloalkyl- or —(CH₂)ₘ— (3- to 10-membered heterocycloalkyl) group; wherein said C₁-C₅-alkyl- group is optionally substituted one, two or three times, identically or differently, with a group selected from: halo-, —N(R⁸)R⁹, —N(R⁸)C(═O)R¹⁰, -azido; wherein said 3- to 10-membered heterocycloalkyl- group is optionally substituted, one time, with a —C(═O)—O—R⁹ group.

In another preferred embodiment, the invention relates to compounds of formula (I), supra, wherein R^{2d} represents an —OR⁵ group; wherein R⁵ represents a C₁-C₅-alkyl-, C₃-C₇-cycloalkyl-, —(CH₂)ₘ— (3- to 10-membered heterocycloalkyl) or trifluoromethyl- group; wherein said C₁-C₅-alkyl- group is optionally substituted once with a group selected from: —N(R⁸)R⁹, —N(R⁸)C(═O)R¹⁰, -azido; wherein said 3- to 10-membered heterocycloalkyl- group is optionally substituted, one time, with a —C(═O)—O—R⁹ group.

In another preferred embodiment, the invention relates to compounds of formula (I), supra, wherein R^{2d} represents an —OR⁵ group; wherein R⁵ represents a C₁-C₅-alkyl-, C₃-C₇-cycloalkyl-, —(CH₂)ₘ— (3- to 7-membered heterocycloalkyl) or trifluoromethyl- group, wherein said C₁-C₅-alkyl- group is substituted once with a group selected from: —N(R⁸)R⁹, —N(R⁸)C(═O)R¹⁰, -azido; wherein said 3- to 7-membered heterocycloalkyl- group is optionally substituted, one time, with a —C(═O)—O—R⁹ group.

In another preferred embodiment, the invention relates to compounds of formula (I), supra, wherein R^{2d} represents an —OR⁵ group, and wherein R⁵ represents a C₁-C₅-alkyl-, C₃-C₇-cycloalkyl-, 3- to 7-membered heterocycloalkyl- or trifluoromethyl- group, wherein said C₁-C₅-alkyl- is optionally substituted once with a group selected from: —N(R⁸)R⁹, 3- to 7-membered heterocycloalkyl-.

In another preferred embodiment, the invention relates to compounds of formula (I), supra, wherein R^{2d} represents an —OR⁵ group, and wherein R⁵ represents a C₁-C₅-alkyl-, C₃-C₇-cycloalkyl-, 3- to 7-membered heterocycloalkyl- or trifluoromethyl- group, wherein said C₁-C₅-alkyl- is optionally substituted once with a —N(R⁸)R⁹ group.

In another preferred embodiment, the invention relates to compounds of formula (I), supra, wherein R^{2d} represents an —OR⁵ group, and wherein R⁵ represents a C₁-C₅-alkyl-, C₃-C₇-cycloalkyl-, 3- to 7-membered heterocycloalkyl- or trifluoromethyl- group, wherein said C₁-C₅-alkyl- is optionally substituted once with a 3- to 7-membered heterocycloalkyl- group.

In another preferred embodiment, the invention relates to compounds of formula (I), supra, wherein R^{2d} represents an —OR⁵ group, and wherein R⁵ represents a C₁-C₅-alkyl-, C₃-C₇-cycloalkyl-, 3- to 7-membered heterocycloalkyl- or trifluoromethyl- group.

In another preferred embodiment, the invention relates to compounds of formula (I), supra, wherein R^{2d} represents an —OR⁵ group; wherein R⁵ represents a —(CH₂)ₘ— (3- to 7-membered heterocycloalkyl) group; wherein said 3- to 7-membered heterocycloalkyl- group is optionally substituted, one time, with a —C(═O)—O—R⁹ group.

In another preferred embodiment, the invention relates to compounds of formula (I), supra, wherein R^{2d} represents an —OR⁵ group, and wherein R⁵ represents a C₁-C₅-alkyl- or C₃-C₇-cycloalkyl- group.

In another preferred embodiment, the invention relates to compounds of formula (I), supra, wherein R^{2d} represents an —OR⁵ group, and wherein R⁵ represents a C₁-C₅-alkyl- group.

In another preferred embodiment, the invention relates to compounds of formula (I), supra, wherein R^{2d} represents C₁-C₃-alkoxy- or halo-C₁-C₃-alkoxy-.

In another preferred embodiment, the invention relates to to the first group of compounds of formula (I), supra, wherein R^{2d} represents a C₁-C₃-alkoxy- or halo-C₁-C₃-alkoxy- group, preferably a methoxy-, ethoxy-, iso-propoxy- or trifluoromethoxy- group.

In another preferred embodiment, the invention relates to compounds of formula (I), supra, wherein R^{2d} represents a C₁-C₃-alkoxy- group, preferably a methoxy-, ethoxy- or iso-propoxy- group.

In another preferred embodiment, the invention relates to compounds of formula (I), supra, wherein R^{2d} represents an iso-propoxy- group.

In another preferred embodiment, the invention relates to compounds of formula (I), supra, wherein R^{2d} represents an ethoxy- group.

In another preferred embodiment, the invention relates to compounds of formula (I), supra, wherein R^{2d} represents a methoxy- group.

In another preferred embodiment, the invention relates to compounds of formula (I), supra, wherein R^{2d} represents a group selected from: —N(H)R⁷, —N(R⁶)R⁷, —N(R⁶)R¹¹.

In another preferred embodiment, the invention relates to compounds of formula (I), supra, wherein R^{2d} represents a —N(H)R⁷ group.

In another preferred embodiment, the invention relates to compounds of formula (I), supra, wherein R^{2d} represents a —N(R⁶)R⁷ group.

In another preferred embodiment, the invention relates to compounds of formula (I), supra, wherein R^{2d} represents —N(R⁶)R¹¹.

In another preferred embodiment, the invention relates to compounds of formula (I), supra, wherein R³ represents a group selected from: C₁-C₆-alkyl-, C₁-C₆-alkoxy-, —(CH₂)_{q}—(C₃-C₇-cycloalkyl), —(CH₂)ₚ—O—(C₃-C₇-cycloalkyl), —(CH₂)_{q}— (3- to 10-membered heterocycloalkyl), —(CH₂)ₚ—O— (3- to 10-membered heterocycloalkyl), —(CH₂)_{q}-aryl, —(CH₂)ₚ—O-aryl, —(CH₂)_{q}-heteroaryl, —(CH₂)ₚ—O-heteroaryl —S(═O)₂—R⁶;

wherein said C₁-C₆-alkyl- group is optionally substituted one, two or three times, identically or differently, with a group selected from: fluoro-, C₁-C₃-alkoxy-, HO—, —N(R⁸)R⁹.

In another preferred embodiment, the invention relates to compounds of formula (I), supra, wherein R³ represents a group selected from: C₁-C₆-alkyl-, C₁-C₃-alkoxy-, C₃-C₇-cycloalkyl-, 4- to 6-membered heterocycloalkyl-, —(CH₂)_{q}-phenyl, —(CH₂)_{q}— (5- or 6-membered heteroaryl), —S(═O)₂—R⁶; wherein said C₁-C₆-alkyl- group is optionally substituted one, two or three times, identically or differently, with a group selected from: fluoro-, C₁-C₃-alkoxy-, HO—, —N(R⁸)R⁹.

In another preferred embodiment, the invention relates to compounds of formula (I), supra, wherein R³ represents a group selected from: C₁-C₆-alkyl-, C₁-C₂-alkoxy-, C₃-C₅-cycloalkyl-, 4- to 6-membered heterocycloalkyl-, benzyl-, —CH₂-(pyridyl), —CH₂-(imidazolyl), —S(═O)₂—CH₃;

wherein said C₁-C₆-alkyl- group is optionally substituted one, two or three times, identically or differently, with a group selected from: fluoro-, methoxy-, HO—, —N(CH₃)CH₃.

In another preferred embodiment, the invention relates to compounds of formula (I), supra, wherein R³ represents a group selected from: C₁-C₆-alkyl-, C₁-C₆-alkoxy-, —(CH₂)_{q}—(C₃-C₇-cycloalkyl), —(CH₂)_{q}— (3- to 10-membered heterocycloalkyl), —(CH₂)_{q}-aryl, —(CH₂)_{q}-heteroaryl, —S(═O)₂—R⁶;

wherein said C₁-C₆-alkyl- group is optionally substituted one or two or three times, identically or differently, with a group selected from: fluoro-, C₁-C₃-alkoxy-, HO—, —N(R⁸)R⁹.

In another preferred embodiment, the invention relates to compounds of formula (I), supra, wherein R³ represents a group selected from:

C₁-C₆-alkyl-, C₁-C₃-alkoxy-, —(CH₂)_{q}—(C₃-C₇-cycloalkyl), —(CH₂)_{q}— (3- to 10-membered heterocycloalkyl), —(CH₂)_{q}-aryl, —(CH₂)_{q}-heteroaryl, —S(═O)₂—R⁶;
wherein said C₁-C₆-alkyl- group is optionally substituted one or two or three times, identically or differently, with a group selected from: fluoro-, C₁-C₃-alkoxy-, HO—, —N(R⁸)R⁹.

In another preferred embodiment, the invention relates to compounds of formula (I), supra, wherein R³ represents a group selected from: —(CH₂)_{q}—(C₃-C₇-cycloalkyl), —(CH₂)ₚ—O—(C₃-C₇-cycloalkyl).

In another preferred embodiment, the invention relates to compounds of formula (I), supra, wherein R³ represents a group selected from:

—(CH₂)_{q}— (3- to 10-membered heterocycloalkyl),
—(CH₂)ₚ—O— (3- to 10-membered heterocycloalkyl).

In another preferred embodiment, the invention relates to compounds of formula (I), supra, wherein R³ represents a group selected from:

—(CH₂)_{q}-aryl, —(CH₂)ₚ—O-aryl, —(CH₂)_{q}-heteroaryl, —(CH₂)ₚ—O-heteroaryl.

In another preferred embodiment, the invention relates to compounds of formula (I), supra, wherein R³ represents a group selected from:

C₁-C₆-alkyl-, —(CH₂)_{q}—(C₃-C₇-cycloalkyl),
—(CH₂)_{q}— (3- to 10-membered heterocycloalkyl), —(CH₂)_{q}-aryl, —(CH₂)_{q}-heteroaryl;
wherein said C₁-C₆-alkyl- group is optionally substituted one or two or three times, identically or differently, with a group selected from: fluoro-, C₁-C₃-alkoxy-, HO—, —N(R⁸)R⁹.

In another preferred embodiment, the invention relates to compounds of formula (I), supra, wherein R³ represents a group selected from:

—(CH₂)_{q}—(C₃-C₇-cycloalkyl), —(CH₂)_{q}— (3- to 10-membered heterocycloalkyl),
—(CH₂)_{q}-aryl, —(CH₂)_{q}-heteroaryl.

In another preferred embodiment, the invention relates to compounds of formula (I), supra, wherein R³ represents a group selected from:

—(CH₂)_{q}— (3- to 10-membered heterocycloalkyl), —(CH₂)_{q}-heteroaryl.

In another preferred embodiment, the invention relates to compounds of formula (I), supra, wherein R³ represents a group selected from:

—(CH₂)_{q}—(C₃-C₇-cycloalkyl), —(CH₂)_{q}— (3- to 10-membered heterocycloalkyl).

In another preferred embodiment, the invention relates to compounds of formula (I), supra, wherein R³ represents a group selected from: —(CH₂)_{q}-aryl,

—(CH₂)_{q}-heteroaryl.

In another preferred embodiment, the invention relates to compounds of formula (I), supra, wherein R³ represents a —(CH₂)_{q}—(C₃-C₇-cycloalkyl) group.

In another preferred embodiment, the invention relates to compounds of formula (I), supra, wherein R³ represents a —(CH₂)_{q}— (3- to 10-membered heterocycloalkyl) group.

In another preferred embodiment, the invention relates to compounds of formula (I), supra, wherein R³ represents a —(CH₂)_{q}-aryl group selected from.

In another preferred embodiment, the invention relates to compounds of formula (I), supra, wherein R³ represents a —(CH₂)_{q}-heteroaryl group selected from.

In another preferred embodiment, the invention relates to compounds of formula (I), supra, wherein R³ represents a C₁-C₆-alkoxy- group.

In another preferred embodiment, the invention relates to compounds of formula (I), supra, wherein R³ represents a C₁-C₃-alkoxy- group.

In another preferred embodiment, the invention relates to compounds of formula (I), supra, wherein R³ represents a methoxy- group.

In another preferred embodiment, the invention relates to compounds of formula (I), supra, wherein R⁴ represents a C₁-C₄-alkyl- group;

wherein said C₁-C₄-alkyl- group is optionally substituted one, two or three times, identically or differently, with a group selected from: fluoro-, HO—, C₁-C₃-alkoxy-, —CN, —N(R⁸)R⁹, —N(R⁷)R⁸, —C(═O)N(R⁸)R⁹, —C(═O)N(R⁷)R⁸.

In another preferred embodiment, the invention relates to compounds of formula (I), supra, wherein R⁴ represents a C₁-C₃-alkyl- group;

wherein said C₁-C₃-alkyl- group is optionally substituted one, two or three times, identically or differently, with a group selected from: fluoro-, HO—, C₁-C₃-alkoxy-, —CN, —N(R⁸)R⁹, —N(R⁷)R⁸, —C(═O)N(R⁸)R⁹, —C(═O)N(R⁷)R⁸.

In another preferred embodiment, the invention relates to compounds of formula (I), supra, wherein R⁴ represents a C₁-C₄-alkyl- group;

wherein said C₁-C₄-alkyl- group is optionally substituted one, two or three times, identically or differently, with a group selected from: fluoro-, HO—, C₁-C₃-alkoxy-, —N(R⁸)R⁹, —N(R⁷)R⁸, —C(═O)N(R⁸)R⁹.

In another preferred embodiment, the invention relates to compounds of formula (I), supra, wherein R⁴ represents a C₁-C₃-alkyl- group;

wherein said C₁-C₃-alkyl- group is optionally substituted one, two or three times, identically or differently, with a group selected from: fluoro-, HO—, C₁-C₃-alkoxy-, —N(R⁸)R⁹, —N(R⁷)R⁸, —C(═O)N(R⁸)R⁹.

In another preferred embodiment, the invention relates to compounds of formula (I), supra, wherein R⁴ represents a C₁-C₃-alkyl- group;

wherein said C₁-C₃-alkyl- group is optionally substituted one, two or three times, identically or differently, with a group selected from: fluoro-, HO—, methoxy-, —N(CH₃)CH₃, —N(R⁷)CH₃, —C(═O)N(CH₃)CH₃,
wherein R⁷ represents a C₂-C₄-alkyl- group, which is substituted once with —N(CH₃)CH₃.

In another preferred embodiment, the invention relates to compounds of formula (I), supra, R⁴ represents a C₁-C₃-alkyl- group;

wherein said C₁-C₃-alkyl- group is optionally substituted one, two or three times, identically or differently, with a group selected from: fluoro-, HO—, C₁-C₃-alkoxy-, —N(R⁸)R⁹, —N(R⁷)R⁸, —C(═O)N(R⁸)R⁹; and wherein R³ represents a C₁-C₃-alkyl- or C₁-C₃-alkoxy- group.

In another preferred embodiment, the invention relates to compounds of formula (I), supra, wherein R³ represents a group selected from:

C₁-C₆-alkyl-, C₁-C₆-alkoxy-, —(CH₂)_{q}—(C₃-C₇-cycloalkyl), —(CH₂)_{q}— (3- to 10-membered heterocycloalkyl), —(CH₂)_{q}-aryl, —(CH₂)_{q}-heteroaryl, —S(═O)₂—R⁶; wherein said C₁-C₆-alkyl- group is optionally substituted one or two or three times, identically or differently, with a group selected from: fluoro-, C₁-C₃-alkoxy-, HO—, —N(R⁸)R⁹; and wherein R⁴ represents a C₁-C₃-alkyl- group.

In another preferred embodiment, the invention relates to compounds of formula (I), supra, R⁴ represents a C₁-C₃-alkyl- group; wherein said C₁-C₃-alkyl- group is optionally substituted one, two or three times, identically or differently, with a group selected from: fluoro-, HO—, C₁-C₃-alkoxy-, —N(R⁸)R⁹, —N(R⁷)R⁸, —C(═O)N(R⁸)R⁹; and wherein R³ represents a C₁-C₃-alkyl- group.

In another preferred embodiment, the invention relates to compounds of formula (I), supra, R⁴ represents a C₁-C₃-alkyl- group;

wherein said C₁-C₃-alkyl- group is optionally substituted one, two or three times, identically or differently, with a group selected from: fluoro-, HO—, C₁-C₃-alkoxy-, —N(R⁸)R⁹, —N(R⁷)R⁸, —C(═O)N(R⁸)R⁹; and wherein R³ represents a methyl- group.

In another preferred embodiment, the invention relates to compounds of formula (I), supra, R⁴ represents a C₁-C₃-alkyl- group;

wherein said C₁-C₃-alkyl- group is optionally substituted one, two or three times, identically or differently, with a group selected from: fluoro-, HO—, methoxy-, —N(CH₃)CH₃, —N(R⁷)CH₃, —C(═O)N(CH₃)CH₃,
wherein R⁷ represents a C₂-C₄-alkyl- group, which is substituted once with —N(CH₃)CH₃;
and in which compounds R³ represents a methyl- group.

In another preferred embodiment, the invention relates to compounds of formula (I), supra, wherein R³ represents a group selected from:

C₁-C₆-alkyl-, —(CH₂)_{q}—(C₃-C₇-cycloalkyl),
—(CH₂)_{q}— (3- to 10-membered heterocycloalkyl), —(CH₂)_{q}-aryl, —(CH₂)_{q}-heteroaryl;
wherein said C₁-C₆-alkyl- group is optionally substituted one or two or three times, identically or differently, with a group selected from: fluoro-, C₁-C₃-alkoxy-, HO—, —N(R⁸)R⁹; and wherein R⁴ represents a methyl- group.

In another preferred embodiment, the invention relates to compounds of formula (I), supra, wherein N(R³)R⁴ together represent a 3- to 10-membered heterocycloalkyl- group;

wherein said 3- to 10-membered heterocycloalkyl- group is optionally substituted one, two or three times, identically or differently, with a group selected from: fluoro-, —OH, —N(R⁷)R⁸, —N(R⁸)R⁹, C₁-C₃-alkyl-, —CN, —C(═O)N(R⁸)R⁹, -aryl, —(C₁-C₃-alkyl)-N(R⁸)R⁹.

In another preferred embodiment, the invention relates to compounds of formula (I), supra, wherein N(R³)R⁴ together represent a 3- to 10-membered heterocycloalkyl- group;

wherein said 3- to 10-membered heterocycloalkyl- group is optionally substituted one or two times, identically or differently, with a group selected from:
fluoro-, —OH, —N(R⁷)R⁸, —N(R⁸)R⁹, C₁-C₃-alkyl-, —CN, —C(═O)N(R⁸)R⁹, -aryl, —(C₁-C₃-alkyl)-N(R⁸)R⁹.

In another preferred embodiment, the invention relates to compounds of formula (I), supra, wherein N(R³)R⁴ together represent a 3- to 10-membered heterocycloalkyl- group;

wherein said 3- to 10-membered heterocycloalkyl- group is optionally substituted one or two times, identically or differently, with a group selected from:
fluoro-, —OH, —N(R⁷)CH₃, —N(CH₃)CH₃, methyl-, —CN, —C(═O)N(CH₃)CH₃, phenyl-, —(C₁-C₃-alkyl)-N(CH₃) CH₃;
wherein R⁷ represents a C₂-C₄-alkyl- group, which is substituted once with —N(CH₃)CH₃.

In another preferred embodiment, the invention relates to compounds of formula (I), supra, wherein N(R³)R⁴ together represent a 5- or 6-membered monocyclic heterocycloalkyl- group;

wherein said 5- or 6-membered monocyclic heterocycloalkyl- group is optionally substituted one or two times, identically or differently, with a group selected from: fluoro-, —OH, —N(R⁷)R⁸, —N(R⁸)R⁹, C₁-C₃-alkyl-, —CN, —C(═O)N(R⁸)R⁹, -aryl, —(C₁-C₃-alkyl)-N(R⁸)R⁹.

In another preferred embodiment, the invention relates to compounds of formula

(I), supra, wherein N(R³)R⁴ together represent a 5- or 6-membered monocyclic heterocycloalkyl- group;

wherein said 5- or 6-membered monocyclic heterocycloalkyl- group is optionally substituted one or two times, identically or differently, with a group selected from:
—N(R⁷)CH₃, —N(CH₃)CH₃, methyl-, —C(═O)N(CH₃)CH₃;
wherein R⁷ represents a C₂-C₄-alkyl- group, which is substituted once with —N(CH₃)CH₃.

In another preferred embodiment, the invention relates to compounds of formula (I), supra, wherein N(R³)R⁴ together represent a 6-membered monocyclic heterocycloalkyl- group selected from piperidinyl-, piperazinyl- and morpholinyl-;

wherein said 6-membered monocyclic heterocycloalkyl- group is optionally substituted one or two times, identically or differently, with a group selected from:
—N(CH₃)CH₃, methyl-.

In another preferred embodiment, the invention relates to compounds of formula (I), supra, wherein N(R³)R⁴ together represent a morpholinyl- group;

wherein said morpholinyl- group is optionally substituted one or two times, identically or differently, with a group selected from:
C₁-C₃-alkyl-.

In another preferred embodiment, the invention relates to compounds of formula (I), supra, wherein R⁵ represents a hydrogen atom or a C₁-C₅-alkyl-, C₃-C₇-cycloalkyl- or —(CH₂)ₘ— (3- to 10-membered heterocycloalkyl) group; wherein said C₁-C₅-alkyl- group is optionally substituted one, two or three times, identically or differently, with a group selected from: halo-, —N(R⁸)R⁹, —N(R⁸)C(═O)R¹⁰, -azido; wherein said 3- to 10-membered heterocycloalkyl- group is optionally substituted, one time, with a —C(═O)—O—R⁹ group.

In another preferred embodiment, the invention relates to compounds of formula (I), supra, wherein R⁵ represents a hydrogen atom or a C₁-C₅-alkyl-, C₃-C₇-cycloalkyl-, —(CH₂)ₘ— (3- to 10-membered heterocycloalkyl) or trifluoromethyl-group, wherein said C₁-C₅-alkyl- group is optionally substituted once with a group selected from: —N(R⁸)R⁹, —N(R⁸)C(═O)R¹⁰, -azido; wherein said 3- to 10-membered heterocycloalkyl- group is optionally substituted, one time, with a —C(═O)—O—R⁹ group.

In another preferred embodiment, the invention relates to compounds of formula (I), supra, wherein R⁵ represents a hydrogen atom or a C₁-C₅-alkyl-, C₃-C₇-cycloalkyl-, —(CH₂)ₘ— (3- to 7-membered heterocycloalkyl) or trifluoromethyl-group, wherein said C₁-C₅-alkyl- group is substituted once with a group selected from: —N(R⁸)R⁹, —N(R⁸)C(═O)R¹⁰, -azido; wherein said 3- to 7-membered heterocycloalkyl- group is optionally substituted, one time, with a —C(═O)—O—R⁹ group.

In another preferred embodiment, the invention relates to compounds of formula (I), supra, wherein R⁵ represents a hydrogen atom or a C₁-C₅-alkyl-, C₃-C₇-cycloalkyl-, —(CH₂)ₘ— (3- to 7-membered heterocycloalkyl) or trifluoromethyl- group, wherein said C₁-C₅-alkyl- is optionally substituted once with a —N(R⁸)R⁹ group; wherein said 3- to 7-membered heterocycloalkyl- group is optionally substituted, one time, with a —C(═O)—O—R⁹ group.

In another preferred embodiment, the invention relates to compounds of formula (I), supra, wherein R⁵ represents a hydrogen atom or a C₁-C₅-alkyl-, C₃-C₇-cycloalkyl-, 3- to 7-membered heterocycloalkyl- or trifluoromethyl- group, wherein said C₁-C₅-alkyl- is optionally substituted once with a —N(R⁸)R⁹ group.

In another preferred embodiment, the invention relates to compounds of formula (I), supra, wherein R⁵ represents a hydrogen atom or a C₁-C₅-alkyl-, C₃-C₇-cycloalkyl-, —(CH₂)ₘ— (3- to 7-membered heterocycloalkyl) or trifluoromethyl- group.

In another preferred embodiment, the invention relates to compounds of formula (I), supra, wherein R⁵ represents a hydrogen atom or a C₁-C₅-alkyl-, C₃-C₇-cycloalkyl-, 3- to 7-membered heterocycloalkyl- or trifluoromethyl- group; wherein said 3- to 7-membered heterocycloalkyl- group is optionally substituted, one time, with a —C(═O)—O—R⁹ group.

In another preferred embodiment, the invention relates to compounds of formula (I), supra, wherein R⁵ represents a C₁-C₅-alkyl- group; wherein said C₁-C₅-alkyl- group is substituted one, two or three times, identically or differently, with a group selected from: —N(R⁸)R⁹, —N(H)C(═O)R¹⁰, -azido; or wherein R⁵ represents a C₃-C₇-cycloalkyl-, 3- to 10-membered heterocycloalkyl- group, or an unsubstituted C₄-C₅-alkyl- or halo-C₄-C₅-alkyl group.

In another preferred embodiment, the invention relates to compounds of formula (I), supra, wherein R⁵ represents a C₁-C₅-alkyl- group; wherein said C₁-C₅-alkyl- is substituted once with a group selected from: —N(R⁸)R⁹, —N(H)C(═O)R¹⁰, -azido; or wherein R⁵ represents a C₃-C₇-cycloalkyl- or 3- to 10-membered heterocycloalkyl-group, or an unsubstituted

C₄-C₅-alkyl- or halo-C₄-C₅-alkyl group.

In another preferred embodiment, the invention relates to compounds of formula (I), supra, wherein R⁵ represents a C₁-C₅-alkyl- group, wherein said C₁-C₅-alkyl- group is substituted once with a group selected from: —N(R⁸)R⁹, —N(H)C(═O)R¹⁰, -azido; or wherein R⁵ represents a C₃-C₇-cycloalkyl-, 3- to 7-membered heterocycloalkyl- or an unsubstituted C₄-C₅-alkyl- group.

In another preferred embodiment, the invention relates to compounds of formula (I), supra, wherein R⁵ represents a C₁-C₅-alkyl- group, wherein said C₁-C₅-alkyl- group is substituted once with a —N(R⁸)R⁹ group; or wherein R⁵ represents a C₃-C₇-cycloalkyl-, 3- to 7-membered heterocycloalkyl- or an unsubstituted C₄-C₅-alkyl- group.

In another preferred embodiment, the invention relates to compounds of formula (I), supra, wherein R⁵ represents a C₄-C₅-alkyl-, C₃-C₇-cycloalkyl- or 3- to 7-membered heterocycloalkyl- group.

In another preferred embodiment, the invention relates to compounds of formula (I), supra, wherein R⁵ represents a C₃-C₇-cycloalkyl- or 3- to 7-membered heterocycloalkyl- group.

In another preferred embodiment, the invention relates to compounds of formula

(I), supra, wherein R⁵ does not represent C₁-C₃-alkyl- or halo-C₁-C₃-alkyl-.

In another preferred embodiment, the invention relates to compounds of formula (I), supra, wherein R⁵ represents a C₁-C₅-alkyl- or C₃-C₇-cycloalkyl- group.

In another preferred embodiment, the invention relates to compounds of formula (I), supra, wherein R⁵ represents a C₁-C₅-alkyl- group.

In another preferred embodiment, the invention relates to compounds of formula (I), supra, wherein R⁵ represents a C₁-C₃-alkyl- group, wherein said C₁-C₃-alkyl- group is optionally substituted one, two or three times, identically or differently, with a halogen atom.

In another preferred embodiment, the invention relates to compounds of formula (I), supra, wherein R⁵ represents a C₁-C₃-alkyl-, difluoromethyl-, trifluoromethyl- or 2,2,2-trifluoroethyl- group.

In another preferred embodiment, the invention relates to compounds of formula (I), supra, wherein R⁵ represents a C₁-C₃-alkyl- group.

In another preferred embodiment, the invention relates to compounds of formula (I), supra, wherein R⁵ represents an iso-propyl- group.

In another preferred embodiment, the invention relates to compounds of formula (I), supra, wherein R⁵ represents an ethyl- group.

In another preferred embodiment, the invention relates to compounds of formula (I), supra, wherein R⁵ represents a methyl- group.

In another preferred embodiment, the invention relates to compounds of formula (I), supra, wherein R⁵ represents a hydrogen atom.

In another preferred embodiment, the invention relates to compounds of formula (I), supra, wherein R⁵ represents a —(CH₂)ₘ— (3- to 10-membered heterocycloalkyl) group; wherein said 3- to 10-membered heterocycloalkyl- group is optionally substituted, one time, with a —C(═O)—O—R⁹ group.

In another preferred embodiment, the invention relates to compounds of formula (I), supra, wherein R⁶ represents a hydrogen atom or a C₁-C₄-alkyl- group.

In another preferred embodiment, the invention relates to compounds of formula (I), supra, wherein R⁶ represents a hydrogen atom or a C₁-C₂-alkyl- group.

In another preferred embodiment, the invention relates to compounds of formula (I), supra, wherein R⁶ represents a hydrogen atom.

In another preferred embodiment, the invention relates to compounds of formula (I), supra, wherein R⁶ represents a C₁-C₄-alkyl- group.

In another preferred embodiment, the invention relates to compounds of formula (I), supra, wherein R⁶ represents a C₁-C₂-alkyl- group.

In another preferred embodiment, the invention relates to compounds of formula (I), supra, wherein R⁷ represents a C₁-C₄-alkyl- or C₃-C₄-alkenyl- group; wherein said C₁-C₄-alkyl- is optionally substituted once with —OH, —N(R⁸)R⁹.

In another preferred embodiment, the invention relates to compounds of formula (I), supra, wherein R⁷ represents a C₁-C₄-alkyl- or C₃-C₄-alkenyl- group, wherein said C₁-C₄-alkyl- is optionally substituted once with —OH, —N(R⁸)R⁹.

In another preferred embodiment, the invention relates to compounds of formula (I), supra, wherein R⁷ represents a C₁-C₄-alkyl- group, wherein said C₁-C₄-alkyl- is optionally substituted once with —OH, —N(R⁸)R⁹.

In another preferred embodiment, the invention relates to compounds of formula (I), supra, wherein R⁷ represents a C₁-C₄-alkyl- group.

In another preferred embodiment, the invention relates to compounds of formula (I), supra, wherein R⁷ represents a C₃-C₄-alkenyl- group.

In another preferred embodiment, the invention relates to compounds of formula (I), supra, wherein R⁷ represents a C₁-C₃-alkoxy- group.

In another preferred embodiment, the invention relates to compounds of formula (I), supra, wherein N(R⁶)R⁷ together represent a 3- to 10-membered heterocycloalkyl group; wherein said 3- to 10-membered heterocycloalkyl- group is optionally substituted once with —N(R⁸)R⁹.

In another preferred embodiment, the invention relates to compounds of formula (I), supra, wherein N(R⁶)R⁷ together represent a 3- to 7-membered heterocycloalkyl- group; wherein said 3- to 7-membered heterocycloalkyl- group is optionally substituted once with —N(R⁸)R⁹.

In another preferred embodiment, the invention relates to compounds of formula (I), supra, wherein N(R⁶)R⁷ together represent a 3- to 7-membered heterocycloalkyl group; wherein said 3- to 7-membered heterocycloalkyl- group is optionally substituted once with —N(R⁸)R⁹; and wherein N(R⁸)R⁹ together represent a 3- to 7-membered heterocycloalkyl- group.

In another preferred embodiment, the invention relates to compounds of formula (I), supra, wherein R⁸ represents a hydrogen atom or a C₁-C₄-alkyl- group.

In another preferred embodiment, the invention relates to compounds of formula (I), supra, wherein R⁸ represents a hydrogen atom or a C₁-C₂-alkyl- group.

In another preferred embodiment, the invention relates to compounds of formula (I), supra, wherein R⁸ represents a hydrogen atom.

In another preferred embodiment, the invention relates to compounds of formula (I), supra, wherein R⁸ represents a C₁-C₄-alkyl- group.

In another preferred embodiment, the invention relates to compounds of formula (I), supra, wherein R⁸ represents a C₁-C₂-alkyl- group.

In another preferred embodiment, the invention relates to compounds of formula (I), supra, wherein R⁸ represents a methyl- group.

In another preferred embodiment, the invention relates to compounds of formula (I), supra, wherein R⁹ represents a hydrogen atom or a C₁-C₅-alkyl- group.

In another preferred embodiment, the invention relates to compounds of formula (I), supra, wherein R⁹ represents a hydrogen atom or a C₁-C₄-alkyl- group.

In another preferred embodiment, the invention relates to compounds of formula (I), supra, wherein R⁹ represents a hydrogen atom or a C₁-C₂-alkyl- group.

In another preferred embodiment, the invention relates to compounds of formula (I), supra, wherein R⁹ represents a hydrogen atom.

In another preferred embodiment, the invention relates to compounds of formula (I), supra, wherein R⁹ represents a C₁-C₄-alkyl- group.

In another preferred embodiment, the invention relates to compounds of formula (I), supra, wherein R⁹ represents a C₁-C₂-alkyl- group.

In another preferred embodiment, the invention relates to compounds of formula (I), supra, wherein R⁹ represents a methyl- group.

In another preferred embodiment, the invention relates to compounds of formula (I), supra, wherein R⁸ represents a C₁-C₂-alkyl- group and R⁹ represents a C₁-C₂-alkyl- group.

In another preferred embodiment, the invention relates to compounds of formula (I), supra, wherein R⁸ represents a C₁-C₂-alkyl- group and R⁹ represents a hydrogen atom.

In another preferred embodiment, the invention relates to compounds of formula (I), supra, wherein R⁸ represents a methyl- group and R⁹ represents a methyl- group.

In another preferred embodiment, the invention relates to compounds of formula (I), supra, wherein R⁸ represents a methyl- group and R⁹ represents a hydrogen atom.

In another preferred embodiment, the invention relates to compounds of formula (I), supra, wherein R⁸ represents a hydrogen atom and R⁹ represents a hydrogen atom.

In another preferred embodiment, the invention relates to compounds of formula (I), supra, wherein N(R⁸)R⁹ together represent a 3- to 7-membered heterocycloalkyl- group; wherein said 3- to 7-membered heterocycloalkyl- group is optionally substituted one, two or three times, identically or differently, with a group selected from: halo-, —OH, —N(R⁷)R⁸, C₁-C₃-alkyl-.

In another preferred embodiment, the invention relates to compounds of formula (I), supra, wherein N(R⁸)R⁹ together represent a 3- to 7-membered heterocycloalkyl- group; wherein said 3- to 7-membered heterocycloalkyl- group is optionally substituted one, two or three times, identically or differently, with a group selected from: —OH, —N(R⁷)R⁸, C₁-C₃-alkyl-.

In another preferred embodiment, the invention relates to compounds of formula (I), supra, wherein N(R⁸)R⁹ together represent a 3- to 7-membered heterocycloalkyl- group; wherein said 3- to 7-membered heterocycloalkyl- group is optionally substituted one, two or three times, identically or differently, with a group selected from: -halo.

In another preferred embodiment, the invention relates to compounds of formula (I), supra, wherein N(R⁸)R⁹ together represent a 3- to 7-membered heterocycloalkyl- group.

In another preferred embodiment, the invention relates to compounds of formula (I), supra, wherein R¹⁰ represents a C₁-C₅-alkyl- or a C₁-C₅-alkoxy- group.

In another preferred embodiment, the invention relates to compounds of formula (I), supra, wherein R¹⁰ represents a C₁-C₄-alkyl- or a C₁-C₄-alkoxy- group.

In another preferred embodiment, the invention relates to compounds of formula (I), supra, wherein R¹⁰ represents a C₁-C₄-alkoxy- group.

In another preferred embodiment, the invention relates to compounds of formula (I), supra, wherein R¹⁰ represents a —(CH₂)ₘ—(C₃-C₇-cycloalkyl) group.

In another preferred embodiment, the invention relates to compounds of formula (I), supra, wherein R¹¹ represents a C₁-C₅-alkyl- group; wherein said C₁-C₅-alkyl- group is optionally substituted one, two or three times, identically or differently, with a halogen atom or a group selected from: cyano, —N(R⁸)R⁹, —N(R⁸)C(═O)R¹⁰.

In another preferred embodiment, the invention relates to compounds of formula (I), supra, wherein R¹¹ represents a C₁-C₅-alkyl- group; wherein said C₁-C₅-alkyl- group is optionally substituted one time with a group selected from: cyano, —N(R⁸)R⁹, —N(R⁸)C(═O)R¹⁰.

In another preferred embodiment, the invention relates to compounds of formula (I), supra, wherein m represents 0.

In another preferred embodiment, the invention relates to compounds of formula (I), supra, wherein m represents 1.

In another preferred embodiment, the invention relates to compounds of formula (I), supra, wherein n represents 1.

In another preferred embodiment, the invention relates to compounds of formula (I), supra, wherein n represents 2.

In a further embodiment of the above-mentioned aspect, the invention relates to compounds of formula (I), according to any of the above-mentioned embodiments, in the form of a tautomer, an N-oxide, a hydrate, a solvate, or a salt thereof, or a mixture of same.

It is to be understood that the present invention relates also to any combination of the preferred embodiments described above.

Some examples of combinations are given hereinafter. However, the invention is not limited to these combinations.

In a preferred embodiment, the present invention relates to compounds of general formula (I):

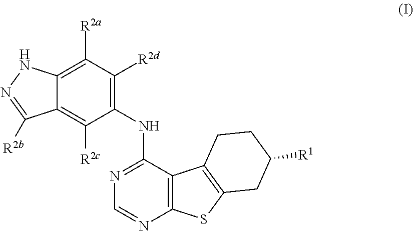

in which:
R¹ represents —C(═O)N(R³)R⁴;
R^{2a} represents a hydrogen atom;
R^{2b} represents a hydrogen atom;
R^{2c} represents a hydrogen atom or a halogen atom;
R^{2d} represents a hydrogen atom, a halogen atom, or a group selected from: cyano-, —OR⁵, —SR⁶, —S(═O)₂R⁶, —S(═O)(═NH)R⁶, —N(H)R⁷, —N(R⁶)R⁷;
R³ represents a group selected from:
   C₁-C₆-alkyl-, C₁-C₆-alkoxy-, C₃-C₆-alkenyl-, C₃-C₆-alkynyl-, —(CH₂)_{q}—(C₃-C₇-cycloalkyl), —(CH₂)ₚ—O—(C₃-C₇-cycloalkyl), —(CH₂)_{q}—(C₄-C₇-cycloalkenyl), —(CH₂)ₚ—O—(C₄-C₇-cycloalkenyl), —(CH₂)_{q}— (3- to 10-membered heterocycloalkyl), —(CH₂)ₚ—O— (3- to 10-membered heterocycloalkyl), —(CH₂)_{q}— (4- to 10-membered heterocycloalkenyl), —(CH₂)ₚ—O— (4- to 10-membered heterocycloalkenyl), —(CH₂)_{q}-aryl, —(CH₂)ₚ—O-aryl, —(CH₂)_{q}-heteroaryl, —(CH₂)ₚ—O-heteroaryl;
   wherein said C₁-C₆-alkyl- group is optionally substituted one, two or three times, identically or differently, with a group selected from:
   halo-, C₁-C₃-alkoxy-, HO—, —N(R⁸)R⁹;
R⁴ represents a C₁-C₄-alkyl- group;
   wherein said C₁-C₄-alkyl- group is optionally substituted one, two or three times, identically or differently, with a halogen atom or a group selected from: HO—, C₁-C₃-alkoxy-, —CN, —N(R⁸)R⁹, —N(R⁷)R⁸, —C(═O)N(R⁸)R⁹, —C(═O)N(R⁷)R⁸;
or
N(R³)R⁴ together
   represent a 3- to 10-membered heterocycloalkyl- or 4- to 10-membered heterocycloalkenyl group;
   wherein said 3- to 10-membered heterocycloalkyl- group is optionally substituted one, two or three times, identically or differently, with a halogen atom or a group selected from: —(CH₂)_{q}—OH, —N(R⁷)R⁸, —N(R⁸)R⁹, C₁-C₃-alkyl-, —CN, —C(═O)R¹⁰, —C(═O)N(R⁸)R⁹, —(CH₂)_{q}-aryl, —(CH₂)_{q}-heteroaryl, —(C₁-C₃-alkyl)-N(R⁸)R⁹;
R⁵ represents a hydrogen atom or a group selected from:
   C₁-C₅-alkyl-, C₃-C₇-cycloalkyl-, 3- to 10-membered heterocycloalkyl-; wherein said C₁-C₅-alkyl- group is optionally substituted one, two or three times, identically or differently, with a halogen atom or a group selected from: cyano, —N(R⁸)R⁹, —N(H)C(═O)R¹⁰, -azido, phenyl-;
R⁶ represents a hydrogen atom or a C₁-C₄-alkyl- group;
R⁷ represents a C₁-C₄-alkyl-, C₃-C₄-alkenyl- or C₁-C₃-alkoxy- group;
   wherein said C₁-C₄-alkyl- is optionally substituted once with —OH or —N(R⁸)R⁹;
or
N(R⁶)R⁷ together
   represent a 3- to 10-membered heterocycloalkyl- or 4- to 10-membered heterocycloalkenyl group;
   wherein said 3- to 10-membered heterocycloalkyl- group is optionally substituted once with —N(R⁸)R⁹;
R⁸ represents a hydrogen atom or a C₁-C₄-alkyl- group;
R⁹ represents a hydrogen atom or a C₁-C₄-alkyl- group;
or
N(R⁸)R⁹ together
   represent a 3- to 10-membered heterocycloalkyl- group;
   wherein said 3- to 10-membered heterocycloalkyl- group is optionally substituted one, two or three times, identically or differently, with a group selected from: —OH, —N(R⁷)R⁸, C₁-C₃-alkyl-;
R¹⁰ represents a C₁-C₄-alkyl- or a C₁-C₄-alkoxy- group;
p represents an integer of 2 or 3; and
q represents an integer of 0, 1, 2 or 3;
or a tautomer, an N-oxide, a hydrate, a solvate, or a salt thereof, or a mixture of same.

In another preferred embodiment, the present invention relates to compounds of general formula (I):

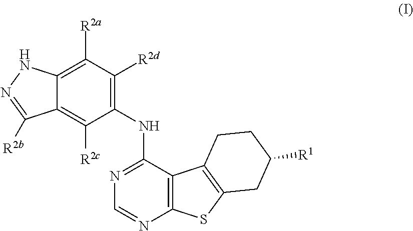

in which:
R¹ represents —C(═O)N(R³)R⁴;
R^{2a} represents a hydrogen atom;
R^{2b} represents a hydrogen atom;
R^{2c} represents a hydrogen atom or a halogen atom;
R^{2d} represents a hydrogen atom, a halogen atom, or a group selected from:
   cyano-, —OR⁵, —SR⁶, —S(═O)₂R⁶, —S(═O)(═NH)R⁶, —N(H)R⁷, —N(R⁶)R⁷, —N(R⁶)R¹¹;
R³ represents a group selected from:
   C₁-C₆-alkyl-, C₁-C₆-alkoxy-, C₃-C₆-alkenyl-, C₃-C₆-alkynyl-, —(CH₂)_{q}—(C₃-C₇-cycloalkyl), —(CH₂)ₚ—O—(C₃-C₇-cycloalkyl), —(CH₂)_{q}—(C₄-C₇-cycloalkenyl), —(CH₂)ₚ—O—(C₄-C₇-cycloalkenyl), —(CH₂)_{q}— (3- to 10-membered heterocycloalkyl), —(CH₂)ₚ—O— (3- to 10-membered heterocycloalkyl), —(CH₂)_{q}— (4- to 10-membered heterocycloalkenyl), —(CH₂)ₚ—O— (4- to 10-membered heterocycloalkenyl), —(CH₂)_{q}-aryl, —(CH₂)ₚ—O-aryl, —(CH₂)_{q}-heteroaryl, —(CH₂)ₚ—O-heteroaryl, —S(═O)₂—R⁶;
   wherein said C₁-C₆-alkyl- group is optionally substituted one, two or three times, identically or differently, with a group selected from:
   halo-, C₁-C₃-alkoxy-, HO—, —N(R⁸)R⁹;
R⁴ represents a C₁-C₄-alkyl- group;
   wherein said C₁-C₄-alkyl- group is optionally substituted one, two or three times, identically or differently, with a halogen atom or a group selected from: HO—, C₁-C₃-alkoxy-, —CN, —N(R⁸)R⁹, —N(R⁷)R⁸, —C(═O)N(R⁸)R⁹, —C(═O)N(R⁷)R⁸;
or
N(R³)R⁴ together
   represent a 3- to 10-membered heterocycloalkyl- or 4- to 10-membered heterocycloalkenyl group;
   wherein said 3- to 10-membered heterocycloalkyl- group is optionally substituted one, two or three times, identically or differently, with a halogen atom or a group selected from: —(CH₂)_{q}—OH, —N(R⁷)R⁸, —N(R⁸)R⁹, C₁-C₃-alkyl-, —CN, —C(═O)R¹⁰, —C(═O)N(R⁸)R⁹, —(CH₂)_{q}-aryl, —(CH₂)_{q}-heteroaryl, —(C₁-C₃-alkyl)-N(R⁸)R⁹;
R⁵ represents a hydrogen atom or a group selected from:
   C₁-C₅-alkyl-, —(CH₂)ₘ—(C₃-C₇-cycloalkyl), —(CH₂)ₘ— (3- to 10-membered heterocycloalkyl);
   wherein said C₁-C₅-alkyl- group is optionally substituted one, two or three times, identically or differently, with a halogen atom or a group selected from: cyano, —N(R⁸)R⁹, —N(R⁸)C(═O)R¹⁰, -azido, phenyl-;
   wherein said C₃-C₇-cycloalkyl- and 3- to 10-membered heterocycloalkyl-group are optionally substituted, one time, with a group selected from: cyano, —N(R⁸)R⁹, —C(═O)—O—R⁹;
R⁶ represents a hydrogen atom or a C₁-C₄-alkyl- group;
R⁷ represents a C₁-C₄-alkyl- or C₃-C₄-alkenyl- group;
   wherein said C₁-C₄-alkyl- is optionally substituted once with —OH or —N(R⁸)R⁹;
or
N(R⁶)R⁷ together
   represent a 3- to 10-membered heterocycloalkyl- or 4- to 10-membered heterocycloalkenyl group;
   wherein said 3- to 10-membered heterocycloalkyl- group is optionally substituted once with —N(R⁸)R⁹;
R⁸ represents a hydrogen atom or a C₁-C₄-alkyl- group;
R⁹ represents a hydrogen atom or a C₁-C₆-alkyl- group;
or
N(R⁸)R⁹ together
   represent a 3- to 10-membered heterocycloalkyl- group;
   wherein said 3- to 10-membered heterocycloalkyl- group is optionally substituted one, two or three times, identically or differently, with a group selected from: halo-, —OH, —N(R⁷)R⁸, C₁-C₃-alkyl-;
R¹⁰ represents a —(CH₂)ₘ—(C₃-C₇-cycloalkyl), C₁-C₆-alkyl- or a C₁-C₆-alkoxy- group;
R¹¹ represents a C₁-C₅-alkyl- group; wherein said C₁-C₅-alkyl- group is optionally substituted one, two or three times, identically or differently, with a halogen atom or a group selected from: cyano, —N(R⁸)R⁹, —N(R⁸)C(═O)R¹⁰;
m represent an integer of 0 or 1;
p represents an integer of 2 or 3; and
q represents an integer of 0, 1, 2 or 3;
or a tautomer, an N-oxide, a hydrate, a solvate, or a salt thereof, or a mixture of same.

In another preferred embodiment, the present invention relates to compounds of general formula (I):

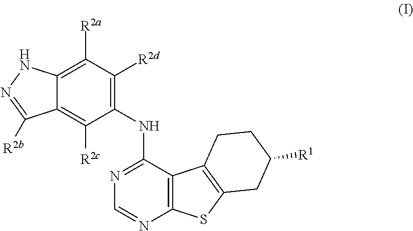

in which:
R¹ represents —C(═O)N(R³)R⁴;
R^{2a} represents a hydrogen atom;
R^{2b} represents a hydrogen atom;
R^{2c} represents a hydrogen atom or a halogen atom;
R^{2d} represents a hydrogen atom, a halogen atom, or a group selected from:
   cyano-, —OR⁵, —SR⁶, —S(═O)₂R⁶, —N(H)R⁷, —N(R⁶)R⁷, —N(R⁶)R¹¹;
R³ represents a group selected from:
   C₁-C₆-alkyl-, C₁-C₆-alkoxy-, —(CH₂)_{q}—(C₃-C₇-cycloalkyl), —(CH₂)ₚ—O—(C₃-C₇-cycloalkyl), —(CH₂)_{q}— (3- to 10-membered heterocycloalkyl), —(CH₂)ₚ—O— (3- to 10-membered heterocycloalkyl), —(CH₂)_{q}-aryl, —(CH₂)ₚ—O-aryl, —(CH₂)_{q}-heteroaryl, —(CH₂)ₚ—O-heteroaryl, —S(═O)₂—R⁶;
   wherein said C₁-C₆-alkyl- group is optionally substituted one, two or three times, identically or differently, with a group selected from: fluoro-, C₁-C₃-alkoxy-, HO—, —N(R⁸)R⁹;
R⁴ represents a C₁-C₄-alkyl- group;
   wherein said C₁-C₄-alkyl- group is optionally substituted one, two or three times, identically or differently, with a group selected from: fluoro-, HO—, C₁-C₃-alkoxy-, —CN, —N(R⁸)R⁹, —N(R⁷)R⁸, —C(═O)N(R⁸)R⁹, —C(═O)N(R⁷)R⁸;
or
N(R³)R⁴ together
   represent a 3- to 10-membered heterocycloalkyl- group;
   wherein said 3- to 10-membered heterocycloalkyl- group is optionally substituted one, two or three times, identically or differently, with a group selected from: fluoro-, —OH, —N(R⁷)R⁸, —N(R⁸)R⁹, C₁-C₃-alkyl-, —CN, —C(═O)N(R⁸)R⁹, -aryl, —(C₁-C₃-alkyl)-N(R⁸)R⁹;
R⁵ represents a hydrogen atom or a group selected from:
   C₁-C₅-alkyl-, C₃-C₇-cycloalkyl-, —(CH₂)ₘ— (3- to 10-membered heterocycloalkyl);
   wherein said C₁-C₅-alkyl- group is optionally substituted one, two or three times, identically or differently, with a halogen atom or a group selected from: cyano, —N(R⁸)R⁹, —N(R⁸)C(═O)R¹⁰, -azido;
   wherein said 3- to 10-membered heterocycloalkyl- group is optionally substituted, one time, with a —C(═O)—O—R⁹ group;
R⁶ represents a hydrogen atom or a C₁-C₄-alkyl- group;
R⁷ represents a C₁-C₄-alkyl- or C₃-C₄-alkenyl- group;
   wherein said C₁-C₄-alkyl- is optionally substituted once with —OH or —N(R⁸)R⁹;
or
N(R⁶)R⁷ together
   represent a 3- to 10-membered heterocycloalkyl group;
   wherein said 3- to 10-membered heterocycloalkyl- group is optionally substituted once with —N(R⁸)R⁹;
R⁸ represents a hydrogen atom or a C₁-C₄-alkyl- group;
R⁹ represents a hydrogen atom or a C₁-C₆-alkyl- group;
or
N(R⁸)R⁹ together
   represent a 3- to 10-membered heterocycloalkyl- group;
   wherein said 3- to 10-membered heterocycloalkyl- group is optionally substituted one, two or three times, identically or differently, with a halogen atom;
R¹⁰ represents a —(CH₂)ₘ—(C₃-C₇-cycloalkyl), C₁-C₆-alkyl- or a C₁-C₆-alkoxy- group;
R¹¹ represents a C₁-C₅-alkyl- group; wherein said C₁-C₅-alkyl- group is optionally substituted one time with a group selected from: cyano, —N(R⁸)R⁹, —N(R⁸)C(═O)R¹⁰;
m represents an integer of 1; and
p represents an integer of 2 or 3; and
q represents an integer of 0, 1, 2 or 3;
or a tautomer, an N-oxide, a hydrate, a solvate, or a salt thereof, or a mixture of same.

In yet another preferred embodiment, the present invention relates to compounds of general formula (I):

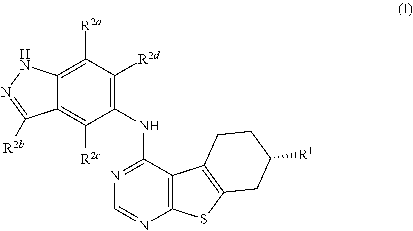

in which:
R¹ represents —C(═O)N(R³)R⁴;
R^{2a} represents a hydrogen atom;
R^{2b} represents a hydrogen atom;
R^{2c} represents a hydrogen atom or a halogen atom;
R^{2d} represents a C₁-C₃-alkoxy- or halo-C₁-C₃-alkoxy- group, preferably a methoxy-, ethoxy-, iso-propoxy- or trifluoromethoxy- group; or a —SR⁶ group;
R³ represents a group selected from:
   C₁-C₆-alkyl-, C₁-C₆-alkoxy-, —(CH₂)_{q}—(C₃-C₇-cycloalkyl), —(CH₂)ₚ—O—(C₃-C₇-cycloalkyl), —(CH₂)_{q}— (3- to 10-membered heterocycloalkyl), —(CH₂)ₚ—O— (3- to 10-membered heterocycloalkyl), —(CH₂)_{q}-aryl, —(CH₂)ₚ—O-aryl, —(CH₂)_{q}-heteroaryl, —(CH₂)ₚ—O-heteroaryl;
   wherein said C₁-C₆-alkyl- group is optionally substituted one, two or three times, identically or differently, with a group selected from: fluoro-, C₁-C₃-alkoxy-, HO—, —N(R⁸)R⁹;
R⁴ represents a C₁-C₄-alkyl- group;
   wherein said C₁-C₄-alkyl- group is optionally substituted one, two or three times, identically or differently, with a halogen atom or a group selected from: HO—, C₁-C₃-alkoxy-, —CN, —N(R⁸)R⁹, —N(R⁷)R⁸, —C(═O)N(R⁸)R⁹, —C(═O)N(R⁷)R⁸;
or
N(R³)R⁴ together
   represent a 3- to 10-membered heterocycloalkyl- or 4- to 10-membered heterocycloalkenyl group;
   wherein said 3- to 10-membered heterocycloalkyl- group is optionally substituted one, two or three times, identically or differently, with a halogen atom or a group selected from: —(CH₂)_{q}—OH, —N(R⁷)R⁸, —N(R⁸)R⁹, C₁-C₃-alkyl-, —CN, —C(═O)R¹⁰, —C(═O)N(R⁸)R⁹, —(CH₂)_{q}-aryl, —(CH₂)_{q}-heteroaryl, —(C₁-C₃-alkyl)-N(R⁸)R⁹;
R⁶ represents a hydrogen atom or a C₁-C₄-alkyl- group;
R⁷ represents a C₁-C₄-alkyl-, C₃-C₄-alkenyl- or C₁-C₃-alkoxy- group;
   wherein said C₁-C₄-alkyl- is optionally substituted once with —OH or —N(R⁸)R⁹;
R⁸ represents a hydrogen atom or a C₁-C₄-alkyl- group;
R⁹ represents a hydrogen atom or a C₁-C₄-alkyl- group;
or
N(R⁸)R⁹ together
   represent a 3- to 10-membered heterocycloalkyl- group;
   wherein said 3- to 10-membered heterocycloalkyl- group is optionally substituted one, two or three times, identically or differently, with a group selected from: —OH, —N(R⁷)R⁸, C₁-C₃-alkyl-;

R¹⁰ represents a C₁-C₄-alkyl- or a C₁-C₄-alkoxy- group;
p represents an integer of 2 or 3; and
q represents an integer of 0, 1, 2 or 3;
or a tautomer, an N-oxide, a hydrate, a solvate, or a salt thereof, or a mixture of same.

In yet another preferred embodiment, the present invention relates to compounds of general formula (I):

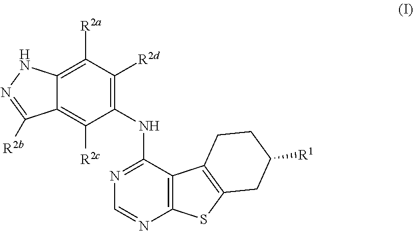

in which:
R¹ represents —C(═O)N(R³)R⁴;
R^{2a} represents a hydrogen atom;
R^{2b} represents a hydrogen atom;
R^{2c} represents a hydrogen atom or a fluoro atom;
R^{2d} represents a hydrogen atom, a halogen atom, or a group selected from: cyano-, —OR⁵, —SR⁶, —S(═O)₂R⁶, —N(H)R⁷, —N(R⁶)R⁷;
R³ represents a group selected from:
   C₁-C₆-alkyl-, C₁-C₃-alkoxy-, —(CH₂)_{q}—(C₃-C₇-cycloalkyl), —(CH₂)_{q}— (3- to 10-membered heterocycloalkyl), —(CH₂)_{q}-aryl, —(CH₂)_{q}-heteroaryl;
   wherein said C₁-C₆-alkyl- group is optionally substituted one or two or three times, identically or differently, with a group selected from:
   fluoro-, C₁-C₃-alkoxy-, HO—, —N(R⁸)R⁹;
R⁴ represents a C₁-C₃-alkyl- group;
   wherein said C₁-C₃-alkyl- group is optionally substituted one, two or three times, identically or differently, with a group selected from:
   fluoro-, HO—, C₁-C₃-alkoxy-, —N(R⁸)R⁹, —N(R⁷)R⁸, —C(═O)N(R⁸)R⁹;
or
N(R³)R⁴ together
   represent a 3- to 10-membered heterocycloalkyl- group;
   wherein said 3- to 10-membered heterocycloalkyl- group is optionally substituted one or two times, identically or differently, with a group selected from: fluoro-, —OH, —N(R⁷)R⁸, —N(R⁸)R⁹, C₁-C₃-alkyl-, —CN, —C(═O)N(R⁸)R⁹, -aryl, —(C₁-C₃-alkyl)-N(R⁸)R⁹;
R⁵ represents a hydrogen atom or group selected from: C₁-C₅-alkyl-, C₃-C₇-cycloalkyl-, 3- to 7-membered heterocycloalkyl-, trifluoromethyl-;
   wherein said C₁-C₅-alkyl- is optionally substituted once with a group selected from: —N(R⁸)R⁹, —N(H)C(═O)R¹⁰, -azido;
R⁶ represents a hydrogen atom or a C₁-C₄-alkyl- group;
R⁷ represents a C₁-C₄-alkyl- or C₃-C₄-alkenyl- group;
   wherein said C₁-C₄-alkyl- is optionally substituted once with —OH or —N(R⁸)R⁹;
or
N(R⁶)R⁷ together
   represent a 3- to 7-membered heterocycloalkyl- group;
   wherein said 3- to 7-membered heterocycloalkyl- group is optionally substituted once with —N(R⁸)R⁹;
R⁸ represents a hydrogen atom or a C₁-C₂-alkyl- group;
R⁹ represents a hydrogen atom or a C₁-C₂-alkyl- group;
or
N(R⁸)R⁹ together
   represent a 3- to 7-membered heterocycloalkyl- group;
R¹⁰ represents a C₁-C₄-alkoxy- group; and
q represents an integer of 0, 1, 2 or 3;
or a tautomer, an N-oxide, a hydrate, a solvate, or a salt thereof, or a mixture of same.

In yet another preferred embodiment, the present invention relates to compounds of general formula (I):

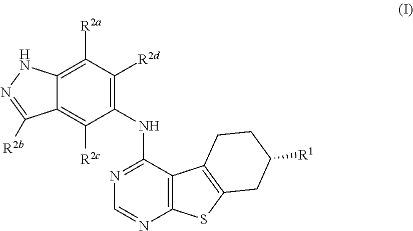

in which:
R¹ represents —C(═O)N(R³)R⁴;
R^{2a} represents a hydrogen atom;
R^{2b} represents a hydrogen atom;
R^{2c} represents a hydrogen atom or a fluoro atom;
R^{2d} represents a hydrogen atom, a halogen atom, or a group selected from: cyano-, —OR⁵, —SR⁶, —N(R⁶)R⁷;
R³ represents a group selected from:
   C₁-C₆-alkyl-, C₁-C₃-alkoxy-, —(CH₂)_{q}—(C₃-C₇-cycloalkyl), —(CH₂)_{q}— (3- to 10-membered heterocycloalkyl), —(CH₂)_{q}-aryl, —(CH₂)_{q}-heteroaryl;
   wherein said C₁-C₆-alkyl- group is optionally substituted one or two or three times, identically or differently, with a group selected from:
   fluoro-, C₁-C₃-alkoxy-, HO—, —N(R⁸)R⁹;
R⁴ represents a C₁-C₃-alkyl- group;
   wherein said C₁-C₃-alkyl- group is optionally substituted one, two or three times, identically or differently, with a group selected from:
   fluoro-, HO—, C₁-C₃-alkoxy-, —N(R⁸)R⁹, —N(R⁷)R⁸, —C(═O)N(R⁸)R⁹;
or
N(R³)R⁴ together
   represent a 3- to 10-membered heterocycloalkyl- group;
   wherein said 3- to 10-membered heterocycloalkyl- group is optionally substituted one or two times, identically or differently, with a group selected from: fluoro-, —OH, —N(R⁷)R⁸, —N(R⁸)R⁹, C₁-C₃-alkyl-, —CN, —C(═O)N(R⁸)R⁹, -aryl, —(C₁-C₃-alkyl)-N(R⁸)R⁹;
R⁵ represents a hydrogen atom or group selected from: C₁-C₅-alkyl-, C₃-C₇-cycloalkyl-, 3- to 7-membered heterocycloalkyl-, trifluoromethyl-;
   wherein said C₁-C₅-alkyl- is optionally substituted once with a group selected from: —N(R⁸)R⁹, —N(H)C(═O)R¹⁰, -azido;
R⁶ represents a hydrogen atom or a C₁-C₄-alkyl- group;
R⁷ represents a C₁-C₄-alkyl- or C₃-C₄-alkenyl- group;
   wherein said C₁-C₄-alkyl- is optionally substituted once with —OH or —N(R⁸)R⁹;
or
N(R⁶)R⁷ together
   represent a 3- to 7-membered heterocycloalkyl- group;
   wherein said 3- to 7-membered heterocycloalkyl- group is optionally substituted once with —N(R⁸)R⁹;
R⁸ represents a hydrogen atom or a C₁-C₂-alkyl- group;
R⁹ represents a hydrogen atom or a C₁-C₂-alkyl- group;
or
N(R⁸)R⁹ together
   represent a 3- to 7-membered heterocycloalkyl- group;
R¹⁰ represents a C₁-C₄-alkoxy- group; and
q represents an integer of 0, 1, 2 or 3;
or a tautomer, an N-oxide, a hydrate, a solvate, or a salt thereof, or a mixture of same.

In another preferred embodiment, the present invention relates to compounds of general formula (I):

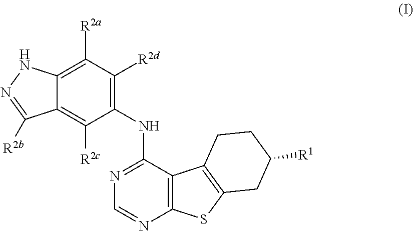

in which:
R¹ represents —C(═O)N(R³)R⁴;
R^{2a} represents a hydrogen atom;
R^{2b} represents a hydrogen atom;
R^{2c} represents a hydrogen atom or a halogen atom;
R^{2d} represents a hydrogen atom, a halogen atom, or a group selected from:
   cyano-, —OR⁵, —N(H)R⁷, —N(R⁶)R⁷;
R³ represents a group selected from:
   C₁-C₆-alkyl-, C₃-C₆-alkenyl-, C₃-C₆-alkynyl-, —(CH₂)_{q}—(C₃-C₇-cycloalkyl), —(CH₂)ₚ—O—(C₃-C₇-cycloalkyl), —(CH₂)_{q}—(C₄-C₇-cycloalkenyl), —(CH₂)ₚ—O—(C₄-C₇-cycloalkenyl), —(CH₂)_{q}— (3- to 10-membered heterocycloalkyl), —(CH₂)ₚ—O— (3- to 10-membered heterocycloalkyl), —(CH₂)_{q}— (4- to 10-membered heterocycloalkenyl), —(CH₂)ₚ—O— (4- to 10-membered heterocycloalkenyl), —(CH₂)_{q}-aryl, —(CH₂)ₚ—O-aryl, —(CH₂)_{q}-heteroaryl, —(CH₂)ₚ—O-heteroaryl,
   wherein said C₁-C₆-alkyl- group is optionally substituted one, two or three times, identically or differently, with a group selected from: halo-, C₁-C₃-alkoxy-, HO—, —N(R⁸)R⁹;
R⁴ represents a C₁-C₄-alkyl- group;
   wherein said C₁-C₄-alkyl- group is optionally substituted one, two or three times, identically or differently, with a group selected from: halo-, HO—, C₁-C₃-alkoxy-, —CN, —N(R⁸)R⁹, —N(R⁷)R⁸, —C(═O)N(R⁸)R⁹, —C(═O)N(R⁷)R⁸;
or
N(R³)R⁴ together
   represent a 3- to 10-membered heterocycloalkyl- or 4- to 10-membered heterocycloalkenyl- group;
   wherein said 3- to 10-membered heterocycloalkyl- group is optionally substituted one, two or three times, identically or differently, with a group selected from: halo-, —(CH₂)_{q}—OH, —N(R⁷)R⁸, —N(R⁸)R⁹, C₁-C₃-alkyl-, —CN, —C(═O)R¹⁰, —C(═O)N(R⁸)R⁹, —(CH₂)_{q}-aryl, —(CH₂)_{q}-heteroaryl, —(C₁-C₃-alkyl)-N(R⁸)R⁹;
R⁵ represents a C₁-C₃-alkyl- group;
   wherein said C₁-C₃-alkyl- group is optionally substituted one, two or three times, identically or differently, with a halogen atom;
R⁶ represents a hydrogen atom or a C₁-C₄-alkyl- group;
R⁷ represents a C₁-C₄-alkyl- or C₃-C₄-alkenyl- group;
   wherein said C₁-C₄-alkyl- is optionally substituted once with —OH or —N(R⁸)R⁹;
or
N(R⁶)R⁷ together
   represent a 3- to 10-membered heterocycloalkyl- or 4- to 10-membered heterocycloalkenyl- group;
   wherein said 3- to 10-membered heterocycloalkyl- group is optionally substituted once with —N(R⁸)R⁹;
R⁸ represents a hydrogen atom or a C₁-C₄-alkyl- group;
R⁹ represents a hydrogen atom or a C₁-C₄-alkyl- group;
or
N(R⁸)R⁹ together
   represent a 3- to 10-membered heterocycloalkyl- group;
   wherein said 3- to 10-membered heterocycloalkyl- group is optionally substituted one, two or three times, identically or differently, with a group selected from: —OH, —N(R⁷)R⁸, C₁-C₃-alkyl-;
R¹⁰ represents a C₁-C₄-alkyl- or a C₁-C₄-alkoxy- group;
p represents an integer of 2 or 3; and
q represents an integer of 0, 1, 2 or 3;
or a tautomer, an N-oxide, a hydrate, a solvate, or a salt thereof, or a mixture of same.

In another preferred embodiment, the present invention relates to compounds of general formula (I):

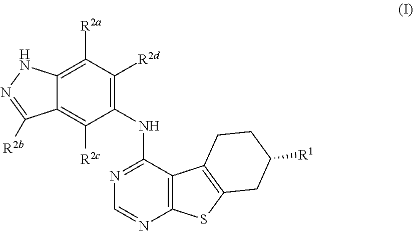

in which:
R¹ represents —C(═O)N(R³)R⁴;
R^{2a} represents a hydrogen atom;
R^{2b} represents a hydrogen atom;
R^{2c} represents a hydrogen atom or a halogen atom;
R^{2d} represents a hydrogen atom, a halogen atom, or a group selected from:
   cyano-, —OR⁵, —N(H)R⁷, —N(R⁶)R⁷;
R³ represents a group selected from:
   C₁-C₆-alkyl-, —(CH₂)_{q}—(C₃-C₇-cycloalkyl), —(CH₂)ₚ—O—(C₃-C₇-cycloalkyl), —(CH₂)_{q}— (3- to 10-membered heterocycloalkyl), —(CH₂)ₚ—O— (3- to 10-membered heterocycloalkyl), —(CH₂)_{q}-aryl, —(CH₂)ₚ—O-aryl, —(CH₂)_{q}-heteroaryl, —(CH₂)ₚ—O-heteroaryl,
   wherein said C₁-C₆-alkyl- group is optionally substituted one, two or three times, identically or differently, with a group selected from: fluoro-, C₁-C₃-alkoxy-, HO—, —N(R⁸)R⁹;
R⁴ represents a C₁-C₄-alkyl- group;
   wherein said C₁-C₄-alkyl- group is optionally substituted one, two or three times, identically or differently, with a group selected from:
   fluoro-, HO—, C₁-C₃-alkoxy-, —CN, —N(R⁸)R⁹, —N(R⁷)R⁸, —C(═O)N(R⁸)R⁹, —C(═O)N(R⁷)R⁸;
or
N(R³)R⁴ together
   represent a 3- to 10-membered heterocycloalkyl- group;
   wherein said 3- to 10-membered heterocycloalkyl- group is optionally substituted one, two or three times, identically or differently, with a group selected from: fluoro-, —OH, —N(R⁷)R⁸, —N(R⁸)R⁹, C₁-C₃-alkyl-, —CN, —C(═O)N(R⁸)R⁹, -aryl, —(C₁-C₃-alkyl)-N(R⁸)R⁹;
R⁵ represents a C₁-C₃-alkyl- group;
   wherein said C₁-C₃-alkyl- group is optionally substituted one, two or three times, identically or differently, with a halogen atom;
R⁶ represents a hydrogen atom or a C₁-C₄-alkyl- group;
R⁷ represents a C₁-C₄-alkyl- or C₃-C₄-alkenyl- group;
   wherein said C₁-C₄-alkyl- is optionally substituted once with —OH or —N(R⁸)R⁹;
or
N(R⁶)R⁷ together
   represent a 3- to 10-membered heterocycloalkyl group;
   wherein said 3- to 10-membered heterocycloalkyl- group is optionally substituted once with —N(R⁸)R⁹;
R⁸ represents a hydrogen atom or a C₁-C₄-alkyl- group;
R⁹ represents a hydrogen atom or a C₁-C₄-alkyl- group;
or
N(R⁸)R⁹ together
   represent a 3- to 10-membered heterocycloalkyl- group;
R¹⁰ represents a C₁-C₄-alkyl- or a C₁-C₄-alkoxy- group;
p represents an integer of 2 or 3; and
q represents an integer of 0, 1, 2 or 3;
or a tautomer, an N-oxide, a hydrate, a solvate, or a salt thereof, or a mixture of same.

In yet another preferred embodiment, the present invention relates to compounds of general formula (I):

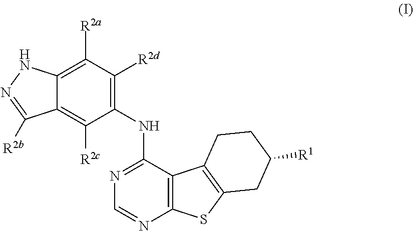

in which:
R¹ represents —C(═O)N(R³)R⁴;
R^{2a} represents a hydrogen atom;
R^{2b} represents a hydrogen atom;
R^{2c} represents a hydrogen atom or a fluoro atom;
R^{2d} represents a hydrogen atom, a halogen atom, or a group selected from:
   cyano-, —OR⁵, —N(H)R⁷, —N(R⁶)R⁷;
R³ represents a group selected from:
   C₁-C₆-alkyl-, —(CH₂)_{q}—(C₃-C₇-cycloalkyl), —(CH₂)_{q}— (3- to 10-membered heterocycloalkyl), —(CH₂)_{q}-aryl, —(CH₂)_{q}-heteroaryl;
   wherein said C₁-C₆-alkyl- group is optionally substituted one or two or three times, identically or differently, with a group selected from: fluoro-, C₁-C₃-alkoxy-, HO—, —N(R⁸)R⁹;
R⁴ represents a C₁-C₃-alkyl- group;
   wherein said C₁-C₃-alkyl- group is optionally substituted one, two or three times, identically or differently, with a group selected from: fluoro-, HO—, C₁-C₃-alkoxy-, —N(R⁸)R⁹, —N(R⁷)R⁸, —C(═O)N(R⁸)R⁹;
or
N(R³)R⁴ together
   represent a 3- to 10-membered heterocycloalkyl- group;
   wherein said 3- to 10-membered heterocycloalkyl- group is optionally substituted one or two times, identically or differently, with a group selected from: fluoro-, —OH, —N(R⁷)R⁸, —N(R⁸)R⁹, C₁-C₃-alkyl-, —CN, —C(═O)N(R⁸)R⁹, -aryl, —(C₁-C₃-alkyl)-N(R⁸)R⁹;
R⁵ represents a C₁-C₃-alkyl-, difluoromethyl-, trifluoromethyl- or 2,2,2-trifluoroethyl- group;
R⁶ represents a hydrogen atom or a C₁-C₄-alkyl- group;
R⁷ represents a C₁-C₄-alkyl- or C₃-C₄-alkenyl- group;
   wherein said C₁-C₄-alkyl- is optionally substituted once with —OH or —N(R⁸)R⁹;
or
N(R⁶)R⁷ together
   represent a 3- to 7-membered heterocycloalkyl- group;
   wherein said 3- to 7-membered heterocycloalkyl- group is optionally substituted once with —N(R⁸)R⁹;
R⁸ represents a hydrogen atom or a C₁-C₂-alkyl- group;
R⁹ represents a hydrogen atom or a C₁-C₂-alkyl- group;
or
N(R⁸)R⁹ together
   represent a 3- to 7-membered heterocycloalkyl- group;
R¹⁰ represents a C₁-C₄-alkoxy- group; and
q represents an integer of 0, 1, 2 or 3;
or a tautomer, an N-oxide, a hydrate, a solvate, or a salt thereof, or a mixture of same.

In another preferred embodiment, the present invention relates to compounds of general formula (I):

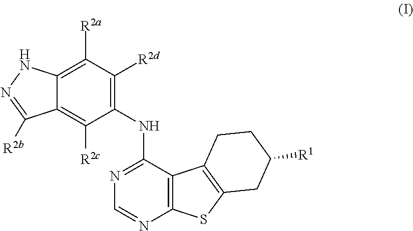

in which:
R¹ represents —C(═O)N(R³)R⁴;
R^{2a} represents a hydrogen atom;
R^{2b} represents a hydrogen atom;
R^{2c} represents a hydrogen atom or a halogen atom;
R^{2d} represents a group selected from:
   —OR⁵, —SR⁶, —S(═O)₂R⁶, —S(═O)(═NH)R⁶;
R³ represents a group selected from:
   C₁-C₆-alkyl-, C₁-C₆-alkoxy-, C₃-C₆-alkenyl-, C₃-C₆-alkynyl-, —(CH₂)_{q}—(C₃-C₇-cycloalkyl), —(CH₂)ₚ—O—(C₃-C₇-cycloalkyl), —(CH₂)_{q}—(C₄-C₇-cycloalkenyl), —(CH₂)ₚ—O—(C₄-C₇-cycloalkenyl), —(CH₂)_{q}— (3- to 10-membered heterocycloalkyl), —(CH₂)ₚ—O— (3- to 10-membered heterocycloalkyl), —(CH₂)_{q}— (4- to 10-membered heterocycloalkenyl), —(CH₂)ₚ—O— (4- to 10-membered heterocycloalkenyl), —(CH₂)_{q}-aryl, —(CH₂)ₚ—O-aryl, —(CH₂)_{q}-heteroaryl, —(CH₂)ₚ—O-heteroaryl,
   wherein said C₁-C₆-alkyl- group is optionally substituted one, two or three times, identically or differently, with a group selected from:
   halo-, C₁-C₃-alkoxy-, HO—, —N(R⁸)R⁹;
R⁴ represents a C₁-C₄-alkyl- group;
   wherein said C₁-C₄-alkyl- group is optionally substituted one, two or three times, identically or differently, with a group selected from: halo-, HO—, C₁-C₃-alkoxy-, —CN, —N(R⁸)R⁹, —N(R⁷)R⁸, —C(═O)N(R⁸)R⁹, —C(═O)N(R⁷)R⁸;
or
N(R³)R⁴ together
   represent a 3- to 10-membered heterocycloalkyl- or 4- to 10-membered heterocycloalkenyl- group;
   wherein said 3- to 10-membered heterocycloalkyl- group is optionally substituted one, two or three times, identically or differently, with a group selected from: halo-, —(CH₂)_{q}—OH, —N(R⁷)R⁸, —N(R⁸)R⁹, C₁-C₃-alkyl-, —CN, —C(═O)R¹⁰, —C(═O)N(R⁸)R⁹, —(CH₂)_{q}-aryl, —(CH₂)_{q}-heteroaryl, —(C₁-C₃-alkyl)-N(R⁸)R⁹;
R⁵ represents a C₁-C₅-alkyl- group, wherein said C₁-C₅-alkyl- group is substituted one, two or three times, identically or differently, with a group selected from: cyano, —N(R⁸)R⁹, —N(H)C(═O)R¹⁰, 3- to 10-membered heterocycloalkyl-, -azido;
or
R⁵ represents a C₃-C₇-cycloalkyl- or 3- to 10-membered heterocycloalkyl- group,
   or an unsubstituted C₄-C₅-alkyl- or halo-C₄-C₅-alkyl- group;
R⁶ represents a hydrogen atom or a C₁-C₄-alkyl- group;
R⁷ represents a C₁-C₄-alkyl- or C₃-C₄-alkenyl- group; wherein said C₁-C₄-alkyl- is optionally substituted once with —OH or —N(R⁸)R⁹;
or
N(R⁶)R⁷ together
   represent a 3- to 10-membered heterocycloalkyl- or 4- to 10-membered heterocycloalkenyl- group;
   wherein said 3- to 10-membered heterocycloalkyl- group is optionally substituted once with —N(R⁸)R⁹;
R⁸ represents a hydrogen atom or a C₁-C₄-alkyl- group;
R⁹ represents a hydrogen atom or a C₁-C₄-alkyl- group;
or
N(R⁸)R⁹ together
   represent a 3- to 10-membered heterocycloalkyl- group;
   wherein said 3- to 10-membered heterocycloalkyl- group is optionally substituted one, two or three times, identically or differently, with a group selected from: —OH, —N(R⁷)R⁸, C₁-C₃-alkyl-;
R¹⁰ represents a C₁-C₄-alkyl- or a C₁-C₄-alkoxy- group;
p represents an integer of 2 or 3; and
q represents an integer of 0, 1, 2 or 3;
or a tautomer, an N-oxide, a hydrate, a solvate, or a salt thereof, or a mixture of same.

In another preferred embodiment, the present invention relates to compounds of general formula (I):

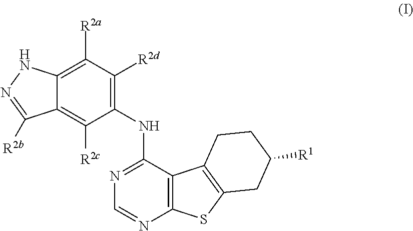

in which:
R¹ represents —C(═O)N(R³)R⁴;
R^{2a} represents a hydrogen atom;
R^{2b} represents a hydrogen atom;
R^{2c} represents a hydrogen atom or a halogen atom;
R^{2d} represents a group selected from:
   —OR⁵, —SR⁶, —S(═O)₂R⁶;
R³ represents a group selected from:
   C₁-C₆-alkyl-, C₁-C₆-alkoxy-, —(CH₂)_{q}—(C₃-C₇-cycloalkyl), —(CH₂)ₚ—O—(C₃-C₇-cycloalkyl), —(CH₂)_{q}— (3- to 10-membered heterocycloalkyl), —(CH₂)ₚ—O— (3- to 10-membered heterocycloalkyl), —(CH₂)_{q}-aryl, —(CH₂)ₚ—O-aryl, —(CH₂)_{q}-heteroaryl, —(CH₂)ₚ—O-heteroaryl,
   wherein said C₁-C₆-alkyl- group is optionally substituted one, two or three times, identically or differently, with a group selected from:
   fluoro-, C₁-C₃-alkoxy-, HO—, —N(R⁸)R⁹;
R⁴ represents a C₁-C₄-alkyl- group;
   wherein said C₁-C₄-alkyl- group is optionally substituted one, two or three times, identically or differently, with a group selected from:
   fluoro-, HO—, C₁-C₃-alkoxy-, —CN, —N(R⁸)R⁹, —N(R⁷)R⁸, —C(═O)N(R⁸)R⁹, —C(═O)N(R⁷)R⁸;
or
N(R³)R⁴ together
   represent a 3- to 10-membered heterocycloalkyl- group;
   wherein said 3- to 10-membered heterocycloalkyl- group is optionally substituted one, two or three times, identically or differently, with a group selected from: fluoro-, —OH, —N(R⁷)R⁸, —N(R⁸)R⁹, C₁-C₃-alkyl-, —CN, —C(═O)N(R⁸)R⁹, -aryl, —(C₁-C₃-alkyl)-N(R⁸)R⁹;
R⁵ represents a C₁-C₅-alkyl- group, wherein said C₁-C₅-alkyl- group is substituted one, two or three times, identically or differently, with a group selected from: —N(R⁸)R⁹, —N(H)C(═O)R¹⁰, 3- to 10-membered heterocycloalkyl-, -azido,
or
R⁵ represents a C₃-C₇-cycloalkyl- or 3- to 10-membered heterocycloalkyl- group, or an unsubstituted C₄-C₅-alkyl- or halo-C₄-C₅-alkyl group;
R⁶ represents a hydrogen atom or a C₁-C₄-alkyl- group;
R⁷ represents a C₁-C₄-alkyl- or C₃-C₄-alkenyl- group;
   wherein said C₁-C₄-alkyl- is optionally substituted once with —OH or —N(R⁸)R⁹;
or
N(R⁶)R⁷ together
   represent a 3- to 10-membered heterocycloalkyl- group;
   wherein said 3- to 10-membered heterocycloalkyl- group is optionally substituted once with —N(R⁸)R⁹;
R⁸ represents a hydrogen atom or a C₁-C₄-alkyl- group;
R⁹ represents a hydrogen atom or a C₁-C₄-alkyl- group;
or
N(R⁸)R⁹ together
   represent a 3- to 10-membered heterocycloalkyl- group;
R¹⁰ represents a C₁-C₄-alkyl- or a C₁-C₄-alkoxy- group;
p represents an integer of 2 or 3; and
q represents an integer of 0, 1, 2 or 3;
or a tautomer, an N-oxide, a hydrate, a solvate, or a salt thereof, or a mixture of same.

In yet another preferred embodiment, the present invention relates to compounds of general formula (I):

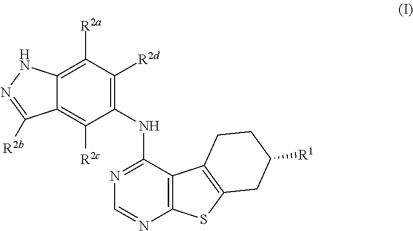

in which:
R¹ represents —C(═O)N(R³)R⁴;
R^{2a} represents a hydrogen atom;
R^{2b} represents a hydrogen atom;
R^{2c} represents a hydrogen atom or a fluoro atom;
R^{2d} represents a group selected from:
   —OR⁵, —SR⁶, —S(═O)₂R⁶;
R³ represents a group selected from:
   C₁-C₆-alkyl-, C₁-C₃-alkoxy-, —(CH₂)_{q}—(C₃-C₇-cycloalkyl), —(CH₂)_{q}— (3- to 10-membered heterocycloalkyl), —(CH₂)_{q}-aryl, —(CH₂)_{q}-heteroaryl;
   wherein said C₁-C₆-alkyl- group is optionally substituted one or two or three times, identically or differently, with a group selected from:
   fluoro-, C₁-C₃-alkoxy-, HO—, —N(R⁸)R⁹;
R⁴ represents a C₁-C₃-alkyl- group;
   wherein said C₁-C₃-alkyl- group is optionally substituted one, two or three times, identically or differently, with a group selected from:
   fluoro-, HO—, C₁-C₃-alkoxy-, —N(R⁸)R⁹, —N(R⁷)R⁸, —C(═O)N(R⁸)R⁹;
or
N(R³)R⁴ together
   represent a 3- to 10-membered heterocycloalkyl- group;
   wherein said 3- to 10-membered heterocycloalkyl- group is optionally substituted one or two times, identically or differently, with a group selected from: fluoro-, —OH, —N(R⁷)R⁸, —N(R⁸)R⁹, C₁-C₃-alkyl-, —CN, —C(═O)N(R⁸)R⁹, -aryl, —(C₁-C₃-alkyl)-N(R⁸)R⁹;
R⁵ represents a C₁-C₅-alkyl- group; wherein said C₁-C₅-alkyl- group is substituted one, two or three times, identically or differently, with a group selected from: —N(R⁸)R⁹, —N(H)C(═O)R¹⁰, 3- to 7-membered heterocycloalkyl-, -azido;
or
R⁵ represents a C₃-C₇-cycloalkyl- or 3- to 7-membered heterocycloalkyl- group, or an unsubstituted C₄-C₅-alkyl- group;
R⁶ represents a hydrogen atom or a C₁-C₄-alkyl- group;
R⁷ represents a C₁-C₄-alkyl- or C₃-C₄-alkenyl- group;
   wherein said C₁-C₄-alkyl- is optionally substituted once with —OH or —N(R⁸)R⁹;
Or
N(R⁶)R⁷ together
   represent a 3- to 7-membered heterocycloalkyl group;
   wherein said 3- to 7-membered heterocycloalkyl- group is optionally substituted once with —N(R⁸)R⁹;
R⁸ represents a hydrogen atom or a C₁-C₂-alkyl- group;
R⁹ represents a hydrogen atom or a C₁-C₂-alkyl- group;
or
N(R⁸)R⁹ together
   represent a 3- to 7-membered heterocycloalkyl- group;
R¹⁰ represents a C₁-C₄-alkoxy- group; and
q represents an integer of 0, 1, 2 or 3;
or a tautomer, an N-oxide, a hydrate, a solvate, or a salt thereof, or a mixture of same.

In yet another preferred embodiment, the present invention relates to compounds of general formula (I):

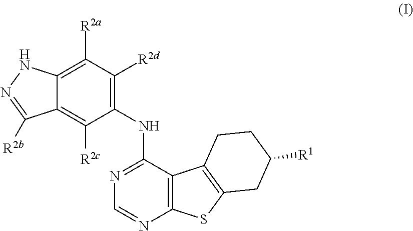

in which:
R¹ represents —C(═O)N(R³)R⁴;
R^{2a} represents a hydrogen atom;
R^{2b} represents a hydrogen atom;
R^{2c} represents a hydrogen atom;
R^{2d} represents a C₁-C₃-alkoxy- group;
N(R³)R⁴ together
   represent a 5- or 6-membered monocyclic heterocycloalkyl- group;
   wherein said 5- or 6-membered monocyclic heterocycloalkyl- group is optionally substituted one or two times, identically or differently, with a group selected from:
   fluoro-, —OH, —N(R⁷)R⁸, —N(R⁸)R⁹, C₁-C₃-alkyl-, —CN, —C(═O)N(R⁸)R⁹, -aryl, —(C₁-C₃-alkyl)-N(R⁸)R⁹;
R⁷ represents a C₁-C₄-alkyl- group;
   wherein said C₁-C₄-alkyl- is optionally substituted once with —OH or —N(R⁸)R⁹;
R⁸ represents a hydrogen atom or a C₁-C₂-alkyl- group;
R⁹ represents a hydrogen atom or a C₁-C₂-alkyl- group;
or
N(R⁸)R⁹ together
   represent a 3- to 7-membered heterocycloalkyl- group;
or a tautomer, an N-oxide, a hydrate, a solvate, or a salt thereof, or a mixture of same.

In yet another preferred embodiment, the present invention relates to compounds of general formula (I):

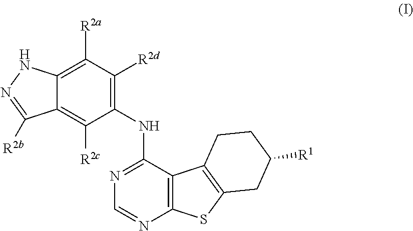

in which:
R¹ represents —C(═O)N(R³)R⁴;
R^{2a} represents a hydrogen atom;
R^{2b} represents a hydrogen atom;
R^{2c} represents a hydrogen atom;
R^{2d} represents a C₁-C₃-alkoxy- group;
N(R³)R⁴ together
   In another preferred embodiment, the invention relates to compounds of formula (I), supra, wherein N(R³)R⁴ together represent a 6-membered monocyclic heterocycloalkyl- group selected from piperidinyl-, piperazinyl- and morpholinyl-;
   wherein said 6-membered monocyclic heterocycloalkyl- group is optionally substituted one or two times, identically or differently, with a group selected from:
   —N(CH₃)CH₃, methyl-;
or a tautomer, an N-oxide, a hydrate, a solvate, or a salt thereof, or a mixture of same.

In yet another preferred embodiment, the invention relates to compounds of formula (I), as defined in any one of the embodiments, wherein the following compounds are excluded:
[(7S)-4-(1H-indazol-5-ylamino)-5,6,7,8-tetrahydro[1]benzothieno[2,3-d]pyrimidin-7-yl](4-methylpiperazin-1-yl)methanone,
(7S)-4-(1H-indazol-5-ylamino)-N,N-dimethyl-5,6,7,8-tetrahydro[1]benzothieno[2,3-d]pyrimidine-7-carboxamide,
(7S)-4-(1H-indazol-5-ylamino)-N-methyl-N-(pyridin-4-ylmethyl)-5,6,7,8-tetrahydro[1]benzothieno[2,3-d]pyrimidine-7-carboxamide,
(7S)-4-(1H-indazol-5-ylamino)-N-methyl-N-(prop-2-yn-1-yl)-5,6,7,8-tetrahydro[1]benzothieno[2,3-d]pyrimidine-7-carboxamide,
(7S)—N-(2-hydroxyethyl)-4-(1H-indazol-5-ylamino)-N-methyl-5,6,7,8-tetrahydro[1]benzothieno[2,3-d]pyrimidine-7-carboxamide,
azetidin-1-yl[(7S)-4-(1H-indazol-5-ylamino)-5,6,7,8-tetrahydro[1]benzothieno[2,3-d]pyrimidin-7-yl]methanone,
(7S)—N-[2-(dimethylamino)ethyl]-4-(1H-indazol-5-ylamino)-N-methyl-5,6,7,8-tetrahydro[1]benzothieno[2,3-d]pyrimidine-7-carboxamide,
[(7S)-4-(1H-indazol-5-ylamino)-5,6,7,8-tetrahydro[1]benzothieno[2,3-d]pyrimidin-7-yl](morpholin-4-yl)methanone,
[(7S)-4-(1H-indazol-5-ylamino)-5,6,7,8-tetrahydro[1]benzothieno[2,3-d]pyrimidin-7-yl](pyrrolidin-1-yl)methanone,
[(7S)-4-(1H-indazol-5-ylamino)-5,6,7,8-tetrahydro[1]benzothieno[2,3-d]pyrimidin-7-yl][4-(pyrrolidin-1-ylcarbonyl)piperazin-1-yl]methanone,
(7S)-4-[(6-methoxy-1H-indazol-5-yl)amino]-N,N-dimethyl-5,6,7,8-tetrahydro[1]benzothieno[2,3-d]pyrimidine-7-carboxamide,
{(7S)-4-[(6-methoxy-1H-indazol-5-yl)amino]-5,6,7,8-tetrahydro[1]benzothieno[2,3-d]pyrimidin-7-yl}(4-methylpiperazin-1-yl)methanone,
(7S)—N-ethyl-N-isopropyl-4-[(6-methoxy-1H-indazol-5-yl)amino]-5,6,7,8-tetrahydro[1]benzothieno[2,3-d]pyrimidine-7-carboxamide,
(7S)-4-[(6-methoxy-1H-indazol-5-yl)amino]-N-methyl-N-propyl-5,6,7,8-tetrahydro[1]benzothieno[2,3-d]pyrimidine-7-carboxamide,
(7S)-4-[(6-isopropoxy-1H-indazol-5-yl)amino]-N,N-dimethyl-5,6,7,8-tetrahydro[1]benzothieno[2,3-d]pyrimidine-7-carboxamide,
{(7S)-4-[(6-isopropoxy-1H-indazol-5-yl)amino]-5,6,7,8-tetrahydro[1]benzothieno[2,3-d]pyrimidin-7-yl}(4-methylpiperazin-1-yl)methanone,
[(7S)-4-(1H-indazol-5-ylamino)-5,6,7,8-tetrahydro[1]benzothieno[2,3-d]pyrimidin-7-yl](piperidin-1-yl)methanone,
(7S)—N,N-diethyl-4-(1H-indazol-5-ylamino)-5,6,7,8-tetrahydro[1]benzothieno[2,3-d]pyrimidine-7-carboxamide,
(7S)—N-benzyl-N-[2-(dimethylamino)ethyl]-4-(1H-indazol-5-ylamino)-5,6,7,8-tetrahydro[1]benzothieno[2,3-d]pyrimidine-7-carboxamide,
(4-hydroxypiperidin-1-yl)[(7S)-4-(1H-indazol-5-ylamino)-5,6,7,8-tetrahydro[1]benzothieno[2,3-d]pyrimidin-7-yl]methanone,
(4-benzylpiperazin-1-yl)[(7S)-4-(1H-indazol-5-ylamino)-5,6,7,8-tetrahydro[1]benzothieno[2,3-d]pyrimidin-7-yl]methanone,
[(7S)-4-(1H-indazol-5-ylamino)-5,6,7,8-tetrahydro[1]benzothieno[2,3-d]pyrimidin-7-yl][4-(pyridin-2-yl)piperazin-1-yl]methanone,
[3-(hydroxymethyl)piperidin-1-yl][(7S)-4-(1H-indazol-5-ylamino)-5,6,7,8-tetrahydro[1]benzothieno[2,3-d]pyrimidin-7-yl]methanone,
(7S)-4-(1H-indazol-5-ylamino)-N-methyl-N-phenyl-5,6,7,8-tetrahydro[1]benzothieno[2,3-d]pyrimidine-7-carboxamide,
(7S)—N-[2-(diethylamino)ethyl]-4-(1H-indazol-5-ylamino)-N-methyl-5,6,7,8-tetrahydro[1]benzothieno[2,3-d]pyrimidine-7-carboxamide,
(7S)—N-[3-(dimethylamino)propyl]-4-(1H-indazol-5-ylamino)-N-methyl-5,6,7,8-tetrahydro[1]benzothieno[2,3-d]pyrimidine-7-carboxamide,
1-(4-{[(7S)-4-(1H-indazol-5-ylamino)-5,6,7,8-tetrahydro[1]benzothieno[2,3-d]pyrimidin-7-yl]carbonyl}piperazin-1-yl)ethanone,
(7S)—N-benzyl-4-(1H-indazol-5-ylamino)-N-methyl-5,6,7,8-tetrahydro[1]benzothieno[2,3-d]pyrimidine-7-carboxamide,
ethyl 4-{[(7S)-4-(1H-indazol-5-ylamino)-5,6,7,8-tetrahydro[1]benzothieno[2,3-d]pyrimidin-7-yl]carbonyl}piperazine-1-carboxylate,
[(7S)-4-(1H-indazol-5-ylamino)-5,6,7,8-tetrahydro[1]benzothieno[2,3-d]pyrimidin-7-yl](3-methylpiperidin-1-yl)methanone,
[(7S)-4-(1H-indazol-5-ylamino)-5,6,7,8-tetrahydro[1]benzothieno[2,3-d]pyrimidin-7-yl](4-methylpiperidin-1-yl)methanone,
[(7S)-4-(1H-indazol-5-ylamino)-5,6,7,8-tetrahydro[1]benzothieno[2,3-d]pyrimidin-7-yl](4-phenylpiperazin-1-yl)methanone,
(7S)-4-(1H-indazol-5-ylamino)-N,N-bis(2-methoxyethyl)-5,6,7,8-tetrahydro[1]benzothieno[2,3-d]pyrimidine-7-carboxamide,
(3-hydroxypiperidin-1-yl)[(7S)-4-(1H-indazol-5-ylamino)-5,6,7,8-tetrahydro[1]benzothieno[2,3-d]pyrimidin-7-yl]methanone,
(7S)—N-ethyl-4-(1H-indazol-5-ylamino)-N-(pyridin-4-ylmethyl)-5,6,7,8-tetrahydro[1]benzothieno[2,3-d]pyrimidine-7-carboxamide,
[(7S)-4-(1H-indazol-5-ylamino)-5,6,7,8-tetrahydro[1]benzothieno[2,3-d]pyrimidin-7-yl][4-(2-methylphenyl)piperazin-1-yl]methanone,
[(7S)-4-(1H-indazol-5-ylamino)-5,6,7,8-tetrahydro[1]benzothieno[2,3-d]pyrimidin-7-yl][4-(3-methylphenyl)piperazin-1-yl]methanone,
[(7S)-4-(1H-indazol-5-ylamino)-5,6,7,8-tetrahydro[1]benzothieno[2,3-d]pyrimidin-7-yl][4-(pyridin-4-yl)piperazin-1-yl]methanone,
[(7S)-4-(1H-indazol-5-ylamino)-5,6,7,8-tetrahydro[1]benzothieno[2,3-d]pyrimidin-7-yl][4-(pyrazin-2-yl)piperazin-1-yl]methanone,
2-(4-{[(7S)-4-(1H-indazol-5-ylamino)-5,6,7,8-tetrahydro[1]benzothieno[2,3-d]pyrimidin-7-yl]carbonyl}piperazin-1-yl)benzonitrile,
[(7S)-4-(1H-indazol-5-ylamino)-5,6,7,8-tetrahydro[1]benzothieno[2,3-d]pyrimidin-7-yl](4-methyl-1,4-diazepan-1-yl)methanone,
(4-ethylpiperazin-1-yl)[(7S)-4-(1H-indazol-5-ylamino)-5,6,7,8-tetrahydro[1]benzothieno[2,3-d]pyrimidin-7-yl]methanone,
(7S)—N-[2-(dimethylamino)-2-oxoethyl]-4-(1H-indazol-5-ylamino)-N-methyl-5,6,7,8-tetrahydro[1]benzothieno[2,3-d]pyrimidine-7-carboxamide,
(7S)—N-(2-hydroxypropyl)-4-(1H-indazol-5-ylamino)-N-methyl-5,6,7,8-tetrahydro[1]benzothieno[2,3-d]pyrimidine-7-carboxamide,
N-[(3R)-1-{[(7S)-4-(1H-indazol-5-ylamino)-5,6,7,8-tetrahydro[1]benzothieno[2,3-d]pyrimidin-7-yl]carbonyl}pyrrolidin-3-yl]acetamide,
4-{[(7S)-4-(1H-indazol-5-ylamino)-5,6,7,8-tetrahydro[1]benzothieno[2,3-d]pyrimidin-7-yl]carbonyl}-N,N-dimethylpiperazine-1-carboxamide,
[(7S)-4-(1H-indazol-5-ylamino)-5,6,7,8-tetrahydro[1]benzothieno[2,3-d]pyrimidin-7-yl](4-phenylpiperidin-1-yl)methanone,
{4-[2-(dimethylamino)ethyl]piperazin-1-yl}[(7S)-4-(1H-indazol-5-ylamino)-5,6,7,8-tetrahydro[1]benzothieno[2,3-d]pyrimidin-7-yl]methanone,
(7S)-4-(1H-indazol-5-ylamino)-N-(2-methoxyethyl)-N-methyl-5,6,7,8-tetrahydro[1]benzothieno[2,3-d]pyrimidine-7-carboxamide,
(4-cyclopentylpiperazin-1-yl)[(7S)-4-(1H-indazol-5-ylamino)-5,6,7,8-tetrahydro[1]benzothieno[2,3-d]pyrimidin-7-yl]methanone,
[4-(hydroxymethyl)piperidin-1-yl][(7S)-4-(1H-indazol-5-ylamino)-5,6,7,8-tetrahydro[1]benzothieno[2,3-d]pyrimidin-7-yl]methanone,
[(7S)-4-(1H-indazol-5-ylamino)-5,6,7,8-tetrahydro[1]benzothieno[2,3-d]pyrimidin-7-yl][4-(2-methoxyethyl)piperazin-1-yl]methanone,
[(3R)-3-hydroxypyrrolidin-1-yl][(7S)-4-(1H-indazol-5-ylamino)-5,6,7,8-tetrahydro[1]benzothieno[2,3-d]pyrimidin-7-yl]methanone,
{4-[2-(1H-imidazol-1-yl)ethyl]piperazin-1-yl}[(7S)-4-(1H-indazol-5-ylamino)-5,6,7,8-tetrahydro[1]benzothieno[2,3-d]pyrimidin-7-yl]methanone,
(7S)-4-(1H-indazol-5-ylamino)-N-methyl-N-[(1-methyl-1H-pyrazol-3-yl)methyl]-5,6,7,8-tetrahydro[1]benzothieno[2,3-d]pyrimidine-7-carboxamide,
(7S)-4-(1H-indazol-5-ylamino)-N-methyl-N-[(1-methyl-1H-pyrazol-5-yl)methyl]-5,6,7,8-tetrahydro[1]benzothieno[2,3-d]pyrimidine-7-carboxamide,
4-(4-{[(7S)-4-(1H-indazol-5-ylamino)-5,6,7,8-tetrahydro[1]benzothieno[2,3-d]pyrimidin-7-yl]carbonyl}piperazin-1-yl)benzonitrile,
[(7S)-4-(1H-indazol-5-ylamino)-5,6,7,8-tetrahydro[1]benzothieno[2,3-d]pyrimidin-7-yl][4-(pyridin-4-ylmethyl)piperazin-1-yl]methanone,
2-(4-{[(7S)-4-(1H-indazol-5-ylamino)-5,6,7,8-tetrahydro[1]benzothieno[2,3-d]pyrimidin-7-yl]carbonyl}piperazin-1-yl)-N,N-dimethylacetamide,
(7S)—N-(3-fluorobenzyl)-4-(1H-indazol-5-ylamino)-N-methyl-5,6,7,8-tetrahydro[1]benzothieno[2,3-d]pyrimidine-7-carboxamide,
[(7S)-4-(1H-indazol-5-ylamino)-5,6,7,8-tetrahydro[1]benzothieno[2,3-d]pyrimidin-7-yl][4-(3-methoxypropyl)piperazin-1-yl]methanone,
[(7S)-4-(1H-indazol-5-ylamino)-5,6,7,8-tetrahydro[1]benzothieno[2,3-d]pyrimidin-7-yl][4-(pyridin-2-ylmethyl)piperazin-1-yl]methanone,
[(7S)-4-(1H-indazol-5-ylamino)-5,6,7,8-tetrahydro[1]benzothieno[2,3-d]pyrimidin-7-yl][4-(pyridin-3-ylmethyl)piperazin-1-yl]methanone,
[(7S)-4-(1H-indazol-5-ylamino)-5,6,7,8-tetrahydro[1]benzothieno[2,3-d]pyrimidin-7-yl][4-(methylsulfonyl)piperazin-1-yl]methanone,
(7S)-4-(1H-indazol-5-ylamino)-N-methyl-N-[(1-methyl-1H-pyrazol-4-yl)methyl]-5,6,7,8-tetrahydro[1]benzothieno[2,3-d]pyrimidine-7-carboxamide,
(3-hydroxyazetidin-1-yl)[(7S)-4-(1H-indazol-5-ylamino)-5,6,7,8-tetrahydro[1]benzothieno[2,3-d]pyrimidin-7-yl]methanone,
methyl 4-{[(7S)-4-(1H-indazol-5-ylamino)-5,6,7,8-tetrahydro[1]benzothieno[2,3-d]pyrimidin-7-yl]carbonyl}piperazine-1-carboxylate,
(7S)-4-(1H-indazol-5-ylamino)-N-methyl-N-(3-thienylmethyl)-5,6,7,8-tetrahydro[1]benzothieno[2,3-d]pyrimidine-7-carboxamide,
(7S)-4-(1H-indazol-5-ylamino)-N-methyl-N-[(1-methyl-1H-pyrrol-2-yl)methyl]-5,6,7,8-tetrahydro[1]benzothieno[2,3-d]pyrimidine-7-carboxamide,
2-(4-{[(7S)-4-(1H-indazol-5-ylamino)-5,6,7,8-tetrahydro[1]benzothieno[2,3-d]pyrimidin-7-yl]carbonyl}piperazin-1-yl)-N-methylacetamide,
N-cyclopropyl-2-(4-{[(7S)-4-(1H-indazol-5-ylamino)-5,6,7,8-tetrahydro[1]benzothieno[2,3-d]pyrimidin-7-yl]carbonyl}piperazin-1-yl)acetamide,
(7S)-4-(1H-indazol-5-ylamino)-N-methyl-N-[2-(pyrrolidin-1-yl)ethyl]-5,6,7,8-tetrahydro[1]benzothieno[2,3-d]pyrimidine-7-carboxamide,
(7S)-4-(1H-indazol-5-ylamino)-N-methyl-N-[2-(4-methylpiperidin-1-yl)ethyl]-5,6,7,8-tetrahydro[1]benzothieno[2,3-d]pyrimidine-7-carboxamide,
(7S)—N-(2,2-difluoroethyl)-4-[(6-methoxy-1H-indazol-5-yl)amino]-N-methyl-5,6,7,8-tetrahydro[1]benzothieno[2,3-d]pyrimidine-7-carboxamide,
(7S)—N-ethyl-N-(2-hydroxyethyl)-4-[(6-methoxy-1H-indazol-5-yl)amino]-5,6,7,8-tetrahydro[1]benzothieno[2,3-d]pyrimidine-7-carboxamide,
(7S)—N-(2-hydroxyethyl)-4-[(6-methoxy-1H-indazol-5-yl)amino]-N-methyl-5,6,7,8-tetrahydro[1]benzothieno[2,3-d]pyrimidine-7-carboxamide,
(7S)—N-isopropyl-4-[(6-methoxy-1H-indazol-5-yl)amino]-N-methyl-5,6,7,8-tetrahydro[1]benzothieno[2,3-d]pyrimidine-7-carboxamide,
1-({(7S)-4-[(6-methoxy-1H-indazol-5-yl)amino]-5,6,7,8-tetrahydro[1]benzothieno[2,3-d]pyrimidin-7-yl}carbonyl)piperidin-4-one,
{(7S)-4-[(6-methoxy-1H-indazol-5-yl)amino]-5,6,7,8-tetrahydro[1]benzothieno[2,3-d]pyrimidin-7-yl}(morpholin-4-yl)methanone,
{(7S)-4-[(6-methoxy-1H-indazol-5-yl)amino]-5,6,7,8-tetrahydro[1]benzothieno[2,3-d]pyrimidin-7-yl}(piperidin-1-yl)methanone,
azetidin-1-yl {(7S)-4-[(6-methoxy-1H-indazol-5-yl)amino]-5,6,7,8-tetrahydro[1]benzothieno[2,3-d]pyrimidin-7-yl}methanone,
[(2R,5R)-2,5-dimethylpyrrolidin-1-yl]{(7S)-4-[(6-methoxy-1H-indazol-5-yl)amino]-5,6,7,8-tetrahydro[1]benzothieno[2,3-d]pyrimidin-7-yl}methanone,
(7S)—N-ethyl-4-[(6-methoxy-1H-indazol-5-yl)amino]-N-methyl-5,6,7,8-tetrahydro[1]benzothieno[2,3-d]pyrimidine-7-carboxamide,
(3,3-dimethylpyrrolidin-1-yl){(7S)-4-[(6-methoxy-1H-indazol-5-yl)amino]-5,6,7,8-tetrahydro[1]benzothieno[2,3-d]pyrimidin-7-yl}methanone,
(7S)—N-cyclopropyl-4-[(6-methoxy-1H-indazol-5-yl)amino]-N-methyl-5,6,7,8-tetrahydro[1]benzothieno[2,3-d]pyrimidine-7-carboxamide,
(7S)—N-(cyclopropylmethyl)-4-[(6-methoxy-1H-indazol-5-yl)amino]-N-methyl-5,6,7,8-tetrahydro[1]benzothieno[2,3-d]pyrimidine-7-carboxamide,
{(7S)-4-[(6-methoxy-1H-indazol-5-yl)amino]-5,6,7,8-tetrahydro[1]benzothieno[2,3-d]pyrimidin-7-yl}(pyrrolidin-1-yl)methanone,
2,5-dihydro-1H-pyrrol-1-yl[(7S)-4-(1H-indazol-5-ylamino)-5,6,7,8-tetrahydro[1]benzothieno[2,3-d]pyrimidin-7-yl]methanone,
(2,6-dimethylmorpholin-4-yl)[(7S)-4-(1H-indazol-5-ylamino)-5,6,7,8-tetrahydro[1]benzothieno[2,3-d]pyrimidin-7-yl]methanone,
[2-(hydroxymethyl)pyrrolidin-1-yl][(7S)-4-(1H-indazol-5-ylamino)-5,6,7,8-tetrahydro[1]benzothieno[2,3-d]pyrimidin-7-yl]methanone,
(7S)-4-(1H-indazol-5-ylamino)-N-isobutyl-N-methyl-5,6,7,8-tetrahydro[1]benzothieno[2,3-d]pyrimidine-7-carboxamide,
(1,1-dioxidothiomorpholin-4-yl)[(7S)-4-(1H-indazol-5-ylamino)-5,6,7,8-tetrahydro[1]benzothieno[2,3-d]pyrimidin-7-yl]methanone,
(7S)-4-(1H-indazol-5-ylamino)-N-methyl-N-[2-(methylamino)-2-oxoethyl]-5,6,7,8-tetrahydro[1]benzothieno[2,3-d]pyrimidine-7-carboxamide,
(7S)—N-(2-cyanoethyl)-N-ethyl-4-(1H-indazol-5-ylamino)-5,6,7,8-tetrahydro[1]benzothieno[2,3-d]pyrimidine-7-carboxamide,
[4-(cyclopropylmethyl)piperazin-1-yl][(7S)-4-(1H-indazol-5-ylamino)-5,6,7,8-tetrahydro[1]benzothieno[2,3-d]pyrimidin-7-yl]methanone,
[(7S)-4-(1H-indazol-5-ylamino)-5,6,7,8-tetrahydro[1]benzothieno[2,3-d]pyrimidin-7-yl](octahydro-2H-pyrido[1,2-a]pyrazin-2-yl)methanone,
(7S)—N-(4-hydroxybutyl)-4-(1H-indazol-5-ylamino)-N-methyl-5,6,7,8-tetrahydro[1]benzothieno[2,3-d]pyrimidine-7-carboxamide,
4-hydroxy-4-(trifluoromethyl)piperidin-1-yl][(7S)-4-(1H-indazol-5-ylamino)-5,6,7,8-tetrahydro[1]benzothieno[2,3-d]pyrimidin-7-yl]methanone,
[(7S)-4-(1H-indazol-5-ylamino)-5,6,7,8-tetrahydro[1]benzothieno[2,3-d]pyrimidin-7-yl](5-methyl-2,5-diazabicyclo[2.2.1]hept-2-yl)methanone,
1-{[(7S)-4-(1H-indazol-5-ylamino)-5,6,7,8-tetrahydro[1]benzothieno[2,3-d]pyrimidin-7-yl]carbonyl}piperidine-3-carbonitrile,
[3-(2-hydroxyethyl)-4-methylpiperazin-1-yl][(7S)-4-(1H-indazol-5-ylamino)-5,6,7,8-tetrahydro[1]benzothieno[2,3-d]pyrimidin-7-yl]methanone,
N-(1-{[(7S)-4-(1H-indazol-5-ylamino)-5,6,7,8-tetrahydro[1]benzothieno[2,3-d]pyrimidin-7-yl]carbonyl}pyrrolidin-3-yl)-N-methylacetamide,
(4,4-difluoropiperidin-1-yl)[(7S)-4-(1H-indazol-5-ylamino)-5,6,7,8-tetrahydro[1]benzothieno[2,3-d]pyrimidin-7-yl]methanone,
[(7S)-4-(1H-indazol-5-ylamino)-5,6,7,8-tetrahydro[1]benzothieno[2,3-d]pyrimidin-7-yl][3-(piperidin-1-yl)azetidin-1-yl]methanone,
2-(4-{[(7S)-4-(1H-indazol-5-ylamino)-5,6,7,8-tetrahydro[1]benzothieno[2,3-d]pyrimidin-7-yl]carbonyl}piperazin-1-yl)-1-(pyrrolidin-1-yl)ethanone,
(7S)—N-(3-hydroxypropyl)-4-(1H-indazol-5-ylamino)-N-methyl-5,6,7,8-tetrahydro[1]benzothieno[2,3-d]pyrimidine-7-carboxamide,
hexahydrocyclopenta[c]pyrrol-2(1H)-yl[(7S)-4-(1H-indazol-5-ylamino)-5,6,7,8-tetrahydro[1]benzothieno[2,3-d]pyrimidin-7-yl]methanone,
[(7S)-4-(1H-indazol-5-ylamino)-5,6,7,8-tetrahydro[1]benzothieno[2,3-d]pyrimidin-7-yl][2-(methoxymethyl)pyrrolidin-1-yl]methanone,
[3-(dimethylamino)pyrrolidin-1-yl][(7S)-4-(1H-indazol-5-ylamino)-5,6,7,8-tetrahydro[1]benzothieno[2,3-d]pyrimidin-7-yl]methanone,
{(7S)-4-[(6-isopropoxy-1H-indazol-5-yl)amino]-5,6,7,8-tetrahydro[1]benzothieno[2,3-d]pyrimidin-7-yl}(2-oxa-6-azaspiro[3.3]hept-6-yl)methanone,
(7S)—N-(2-hydroxyethyl)-N-(2-methoxyethyl)-4-[(6-methoxy-1H-indazol-5-yl)amino]-5,6,7,8-tetrahydro[1]benzothieno[2,3-d]pyrimidine-7-carboxamide,
1-({(7S)-4-[(6-isopropoxy-1H-indazol-5-yl)amino]-5,6,7,8-tetrahydro[1]benzothieno[2,3-d]pyrimidin-7-yl}carbonyl)azetidine-3-carbonitrile,
{(7S)-4-[(6-ethoxy-1H-indazol-5-yl)amino]-5,6,7,8-tetrahydro[1]benzothieno[2,3-d]pyrimidin-7-yl}(piperidin-1-yl)methanone,
1-({(7S)-4-[(6-ethoxy-1H-indazol-5-yl)amino]-5,6,7,8-tetrahydro[1]benzothieno[2,3-d]pyrimidin-7-yl}carbonyl)piperidin-4-one,
{(7S)-4-[(6-ethoxy-1H-indazol-5-yl)amino]-5,6,7,8-tetrahydro[1]benzothieno[2,3-d]pyrimidin-7-yl}(morpholin-4-yl)methanone,
{(7S)-4-[(6-isopropoxy-1H-indazol-5-yl)amino]-5,6,7,8-tetrahydro[1]benzothieno[2,3-d]pyrimidin-7-yl}(piperidin-1-yl)methanone,
(7S)—N-ethyl-N-(2-hydroxyethyl)-4-[(6-isopropoxy-1H-indazol-5-yl)amino]-5,6,7,8-tetrahydro[1]benzothieno[2,3-d]pyrimidine-7-carboxamide,
(7S)-4-[(6-isopropoxy-1H-indazol-5-yl)amino]-N-isopropyl-N-methyl-5,6,7,8-tetrahydro[1]benzothieno[2,3-d]pyrimidine-7-carboxamide,
(7S)—N-(2,2-difluoroethyl)-4-[(6-isopropoxy-1H-indazol-5-yl)amino]-N-methyl-5,6,7,8-tetrahydro[1]benzothieno[2,3-d]pyrimidine-7-carboxamide,
(7S)—N-ethyl-4-[(6-isopropoxy-1H-indazol-5-yl)amino]-N-methyl-5,6,7,8-tetrahydro[1]benzothieno[2,3-d]pyrimidine-7-carboxamide,
{(7S)-4-[(6-isopropoxy-1H-indazol-5-yl)amino]-5,6,7,8-tetrahydro[1]benzothieno[2,3-d]pyrimidin-7-yl}(morpholin-4-yl)methanone,
azetidin-1-yl {(7S)-4-[(6-isopropoxy-1H-indazol-5-yl)amino]-5,6,7,8-tetrahydro[1]benzothieno[2,3-d]pyrimidin-7-yl}methanone,
(7S)—N-(cyclopropylmethyl)-4-[(6-isopropoxy-1H-indazol-5-yl)amino]-N-methyl-5,6,7,8-tetrahydro[1]benzothieno[2,3-d]pyrimidine-7-carboxamide,
{(7S)-4-[(6-isopropoxy-1H-indazol-5-yl)amino]-5,6,7,8-tetrahydro[1]benzothieno[2,3-d]pyrimidin-7-yl}(pyrrolidin-1-yl)methanone,
(1,1-dioxido-1-thia-6-azaspiro[3.3]hept-6-yl){(7S)-4-[(6-isopropoxy-1H-indazol-5-yl)amino]-5,6,7,8-tetrahydro[1]benzothieno[2,3-d]pyrimidin-7-yl}methanone,
azetidin-1-yl {(7S)-4-[(6-ethoxy-1H-indazol-5-yl)amino]-5,6,7,8-tetrahydro[1]benzothieno[2,3-d]pyrimidin-7-yl}methanone,
(7S)-4-[(6-ethoxy-1H-indazol-5-yl)amino]-N-isopropyl-N-methyl-5,6,7,8-tetrahydro[1]benzothieno[2,3-d]pyrimidine-7-carboxamide,
(7S)-4-[(6-ethoxy-1H-indazol-5-yl)amino]-N-(2-hydroxyethyl)-N-methyl-5,6,7,8-tetrahydro[1]benzothieno[2,3-d]pyrimidine-7-carboxamide,
(7S)—N-(2,2-difluoroethyl)-4-[(6-ethoxy-1H-indazol-5-yl)amino]-N-methyl-5,6,7,8-tetrahydro[1]benzothieno[2,3-d]pyrimidine-7-carboxamide,
[(2R,5R)-2,5-dimethylpyrrolidin-1-yl]{(7S)-4-[(6-isopropoxy-1H-indazol-5-yl)amino]-5,6,7,8-tetrahydro[1]benzothieno[2,3-d]pyrimidin-7-yl}methanone,
{(7S)-4-[(6-ethoxy-1H-indazol-5-yl)amino]-5,6,7,8-tetrahydro[1]benzothieno[2,3-d]pyrimidin-7-yl}(pyrrolidin-1-yl)methanone,
(7S)-4-[(6-ethoxy-1H-indazol-5-yl)amino]-N-ethyl-N-methyl-5,6,7,8-tetrahydro[1]benzothieno[2,3-d]pyrimidine-7-carboxamide,
(7S)—N-(2-hydroxyethyl)-4-[(6-isopropoxy-1H-indazol-5-yl)amino]-N-methyl-5,6,7,8-tetrahydro[1]benzothieno[2,3-d]pyrimidine-7-carboxamide,
(7S)-4-[(6-ethoxy-1H-indazol-5-yl)amino]-N-ethyl-N-(2-hydroxyethyl)-5,6,7,8-tetrahydro[1]benzothieno[2,3-d]pyrimidine-7-carboxamide,
1-({(7S)-4-[(6-ethoxy-1H-indazol-5-yl)amino]-5,6,7,8-tetrahydro[1]benzothieno[2,3-d]pyrimidin-7-yl}carbonyl)azetidine-3-carbonitrile,
{(7S)-4-[(6-ethoxy-1H-indazol-5-yl)amino]-5,6,7,8-tetrahydro[1]benzothieno[2,3-d]pyrimidin-7-yl}[(3R)-3-hydroxypyrrolidin-1-yl]methanone,
(7S)—N,N-bis(2-hydroxyethyl)-4-[(6-methoxy-1H-indazol-5-yl)amino]-5,6,7,8-tetrahydro[1]benzothieno[2,3-d]pyrimidine-7-carboxamide,
(7S)—N-cyclopropyl-4-[(6-ethoxy-1H-indazol-5-yl)amino]-N-methyl-5,6,7,8-tetrahydro[1]benzothieno[2,3-d]pyrimidine-7-carboxamide,
(7S)-4-[(6-ethoxy-1H-indazol-5-yl)amino]-N-ethyl-N-isopropyl-5,6,7,8-tetrahydro[1]benzothieno[2,3-d]pyrimidine-7-carboxamide,
1-({(7S)-4-[(6-methoxy-1H-indazol-5-yl)amino]-5,6,7,8-tetrahydro[1]benzothieno[2,3-d]pyrimidin-7-yl}carbonyl)azetidine-3-carbonitrile,
(7S)—N-tert-butyl-4-[(6-ethoxy-1H-indazol-5-yl)amino]-N-methyl-5,6,7,8-tetrahydro[1]benzothieno[2,3-d]pyrimidine-7-carboxamide,
{(7S)-4-[(6-ethoxy-1H-indazol-5-yl)amino]-5,6,7,8-tetrahydro[1]benzothieno[2,3-d]pyrimidin-7-yl}(2-oxa-6-azaspiro[3.3]hept-6-yl)methanone,
(7S)—N-(cyclopropylmethyl)-4-[(6-ethoxy-1H-indazol-5-yl)amino]-N-methyl-5,6,7,8-tetrahydro[1]benzothieno[2,3-d]pyrimidine-7-carboxamide,
1-({(7S)-4-[(6-ethoxy-1H-indazol-5-yl)amino]-5,6,7,8-tetrahydro[1]benzothieno[2,3-d]pyrimidin-7-yl}carbonyl)piperidine-3-carbonitrile,
(7S)—N-(2-cyanoethyl)-4-[(6-ethoxy-1H-indazol-5-yl)amino]-N-ethyl-5,6,7,8-tetrahydro[1]benzothieno[2,3-d]pyrimidine-7-carboxamide,
[(3R,4R)-3,4-dihydroxypyrrolidin-1-yl]{(7S)-4-[(6-methoxy-1H-indazol-5-yl)amino]-5,6,7,8-tetrahydro[1]benzothieno[2,3-d]pyrimidin-7-yl}methanone,
[(3S,4S)-3,4-dihydroxypyrrolidin-1-yl]{(7S)-4-[(6-methoxy-1H-indazol-5-yl)amino]-5,6,7,8-tetrahydro[1]benzothieno[2,3-d]pyrimidin-7-yl}methanone,
(7S)-4-[(6-ethoxy-1H-indazol-5-yl)amino]-N-(2-methoxyethyl)-N-methyl-5,6,7,8-tetrahydro[1]benzothieno[2,3-d]pyrimidine-7-carboxamide,
{(7S)-4-[(6-ethoxy-1H-indazol-5-yl)amino]-5,6,7,8-tetrahydro[1]benzothieno[2,3-d]pyrimidin-7-yl}[(3S)-3-hydroxypyrrolidin-1-yl]methanone,
[4-(cyclopropylmethyl)piperazin-1-yl]{(7S)-4-[(6-ethoxy-1H-indazol-5-yl)amino]-5,6,7,8-tetrahydro[1]benzothieno[2,3-d]pyrimidin-7-yl}methanone,
(7S)-4-{[6-(benzyloxy)-1H-indazol-5-yl]amino}-N,N-dimethyl-5,6,7,8-tetrahydro[1]benzothieno[2,3-d]pyrimidine-7-carboxamide,
(7S)—N,N-dimethyl-4-{[6-(trifluoromethoxy)-1H-indazol-5-yl]amino}-5,6,7,8-tetrahydro[1]benzothieno[2,3-d]pyrimidine-7-carboxamide,
{(7S)-4-[(6-methoxy-1H-indazol-5-yl)amino]-5,6,7,8-tetrahydro[1]benzothieno[2,3-d]pyrimidin-7-yl}(2-oxa-6-azaspiro[3.3]hept-6-yl)methanone,
(7S)-4-{[6-(dimethylamino)-1H-indazol-5-yl]amino}-N,N-dimethyl-5,6,7,8-tetrahydro[1]benzothieno[2,3-d]pyrimidine-7-carboxamide,
(7S)-4-[(6-ethoxy-1H-indazol-5-yl)amino]-N,N-bis(2-methoxyethyl)-5,6,7,8-tetrahydro[1]benzothieno[2,3-d]pyrimidine-7-carboxamide,
(7S)-4-[(6-ethoxy-1H-indazol-5-yl)amino]-N-methyl-N-propyl-5,6,7,8-tetrahydro[1]benzothieno[2,3-d]pyrimidine-7-carboxamide,
(7S)-4-[(6-ethoxy-1H-indazol-5-yl)amino]-N,N-dimethyl-5,6,7,8-tetrahydro[1]benzothieno[2,3-d]pyrimidine-7-carboxamide,
2,5-dihydro-1H-pyrrol-1-yl{(7S)-4-[(6-ethoxy-1H-indazol-5-yl)amino]-5,6,7,8-tetrahydro[1]benzothieno[2,3-d]pyrimidin-7-yl}methanone,
(7S)—N-(2,3-dihydroxypropyl)-4-[(6-ethoxy-1H-indazol-5-yl)amino]-N-methyl-5,6,7,8-tetrahydro[1]benzothieno[2,3-d]pyrimidine-7-carboxamide,
[3-(dimethylamino)pyrrolidin-1-yl]{(7S)-4-[(6-ethoxy-1H-indazol-5-yl)amino]-5,6,7,8-tetrahydro[1]benzothieno[2,3-d]pyrimidin-7-yl}methanone,
{(7S)-4-[(6-isopropoxy-1H-indazol-5-yl)amino]-5,6,7,8-tetrahydro[1]benzothieno[2,3-d]pyrimidin-7-yl}(1-oxa-6-azaspiro[3.3]hept-6-yl)methanone,
{(7S)-4-[(6-methoxy-1H-indazol-5-yl)amino]-5,6,7,8-tetrahydro[1]benzothieno[2,3-d]pyrimidin-7-yl}(1-oxa-6-azaspiro[3.3]hept-6-yl)methanone,
5-azaspiro[2.4]hept-5-yl{(7S)-4-[(6-isopropoxy-1H-indazol-5-yl)amino]-5,6,7,8-tetrahydro[1]benzothieno[2,3-d]pyrimidin-7-yl}methanone,
{(7S)-4-[(6-methoxy-1H-indazol-5-yl)amino]-5,6,7,8-tetrahydro[1]benzothieno[2,3-d]pyrimidin-7-yl}(2-oxa-5-azabicyclo[2.2.1]hept-5-yl)methanone,
(1,1-dioxido-1-thia-6-azaspiro[3.3]hept-6-yl){(7S)-4-[(6-ethoxy-1H-indazol-5-yl)amino]-5,6,7,8-tetrahydro[1]benzothieno[2,3-d]pyrimidin-7-yl}methanone,
(1,1-dioxido-1-thia-6-azaspiro[3.3]hept-6-yl){(7S)-4-[(6-methoxy-1H-indazol-5-yl)amino]-5,6,7,8-tetrahydro[1]benzothieno[2,3-d]pyrimidin-7-yl}methanone,
1-({(7S)-4-[(6-ethoxy-1H-indazol-5-yl)amino]-5,6,7,8-tetrahydro[1]benzothieno[2,3-d]pyrimidin-7-yl}carbonyl)pyrrolidine-3-carbonitrile,
(7S)-4-{[6-(2-chloroethoxy)-1H-indazol-5-yl]amino}-N,N-dimethyl-5,6,7,8-tetrahydro[1]benzothieno[2,3-d]pyrimidine-7-carboxamide,
(7S)-4-{[6-(3-chloropropoxy)-1H-indazol-5-yl]amino}-N,N-dimethyl-5,6,7,8-tetrahydro[1]benzothieno[2,3-d]pyrimidine-7-carboxamide,
tert-butyl {2-[({(7S)-4-[(6-ethoxy-1H-indazol-5-yl)amino]-5,6,7,8-tetrahydro[1]benzothieno[2,3-d]pyrimidin-7-yl}carbonyl)(methyl)amino]ethyl}carbamate,
(7S)—N,N-dimethyl-4-[(6-propoxy-1H-indazol-5-yl)amino]-5,6,7,8-tetrahydro[1]benzothieno[2,3-d]pyrimidine-7-carboxamide,
{(7S)-4-[(6-ethoxy-1H-indazol-5-yl)amino]-5,6,7,8-tetrahydro[1]benzothieno[2,3-d]pyrimidin-7-yl}(octahydro-2H-pyrido[1,2-a]pyrazin-2-yl)methanone,
(7S)—N-(2-aminoethyl)-4-[(6-ethoxy-1H-indazol-5-yl)amino]-N-methyl-5,6,7,8-tetrahydro[1]benzothieno[2,3-d]pyrimidine-7-carboxamide,
(3-hydroxyazetidin-1-yl){(7S)-4-[(6-methoxy-1H-indazol-5-yl)amino]-5,6,7,8-tetrahydro[1]benzothieno[2,3-d]pyrimidin-7-yl}methanone,
(7S)-4-[(6-ethoxy-1H-indazol-5-yl)amino]-N-ethyl-N-(2-methoxyethyl)-5,6,7,8-tetrahydro[1]benzothieno[2,3-d]pyrimidine-7-carboxamide,
5-azaspiro[2.4]hept-5-yl{(7S)-4-[(6-ethoxy-1H-indazol-5-yl)amino]-5,6,7,8-tetrahydro[1]benzothieno[2,3-d]pyrimidin-7-yl}methanone,
1-({(7S)-4-[(6-methoxy-1H-indazol-5-yl)amino]-5,6,7,8-tetrahydro[1]benzothieno[2,3-d]pyrimidin-7-yl}carbonyl)azetidin-3-yl dimethylcarbamate,
(7S)-4-{[6-(3-azidopropoxy)-1H-indazol-5-yl]amino}-N,N-dimethyl-5,6,7,8-tetrahydro[1]benzothieno[2,3-d]pyrimidine-7-carboxamide,
(7S)-4-{[6-(3-aminopropoxy)-1H-indazol-5-yl]amino}-N,N-dimethyl-5,6,7,8-tetrahydro[1]benzothieno[2,3-d]pyrimidine-7-carboxamide,
(7S)-4-{[6-(4-azidobutoxy)-1H-indazol-5-yl]amino}-N,N-dimethyl-5,6,7,8-tetrahydro[1]benzothieno[2,3-d]pyrimidine-7-carboxamide,
7S)-4-{[6-(2-azidoethoxy)-1H-indazol-5-yl]amino}-N,N-dimethyl-5,6,7,8-tetrahydro[1]benzothieno[2,3-d]pyrimidine-7-carboxamide,
[(7S)-4-{[6-(3-chloropropoxy)-1H-indazol-5-yl]amino}-5,6,7,8-tetrahydro[1]benzothieno[2,3-d]pyrimidin-7-yl](4-methylpiperazin-1-yl)methanone.

It is to be understood that the present invention relates to any sub-combination within any embodiment or aspect of the present invention of compounds of general formula (I), supra.

More particularly still, the present invention covers compounds of general formula (I) which are disclosed in the Examples section of this text, infra.

In accordance with another aspect, the present invention covers methods of preparing compounds of the present invention, said methods comprising the steps as described in the Experimental Section herein.

In a preferred embodiment, the present invention relates to a method of preparing compounds of general formula (I), supra, in which method an intermediate compound of general formula (VII):

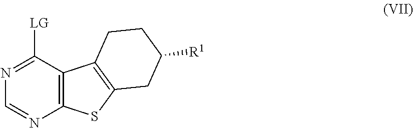

in which R¹ is as defined for the compounds of general formula (I), supra, and LG represents a leaving group (as defined hereinafter);
is allowed to react with a compound of general formula (II):

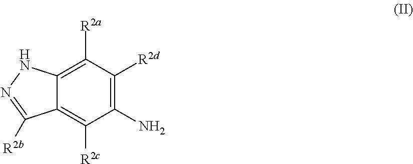

in which R^{2a}, R^{2b}, R^{2c}, and R^{2d} are as defined for the compounds of general formula (I), supra;
thus providing a compound of general formula (I):

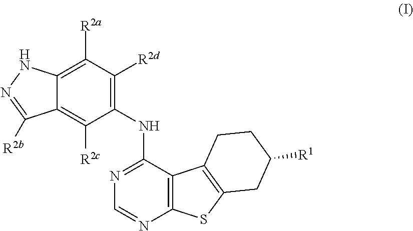

in which R¹, R^{2a}, R^{2b}, R^{2c}, and R^{2d} are as defined for the compounds of general formula (I), supra.

As used herein, the term “leaving group” refers to an atom or a group of atoms that is displaced in a chemical reaction as stable species taking with it the bonding electrons. Preferably, a leaving group is selected from the group comprising: halo, in particular chloro, bromo or iodo, methanesulfonyloxy, p-toluenesulfonyloxy, trifluoromethanesulfonyloxy, nonafluorobutanesulfonyloxy, (4-bromo-benzene)sulfonyloxy, (4-nitro-benzene)sulfonyloxy, (2-nitro-benzene)-sulfonyloxy, (4-isopropyl-benzene)sulfonyloxy, (2, 4, 6-tri-isopropyl-benzene)-sulfonyloxy, (2,4,6-trimethyl-benzene)sulfonyloxy, (4-tert-butyl-benzene)sulfonyloxy, benzenesulfonyloxy, and (4-methoxy-benzene)sulfonyloxy.

In another preferred embodiment, the present invention relates to a method of preparing compounds of general formula (I), supra;

in which method an intermediate compound of general formula (V):

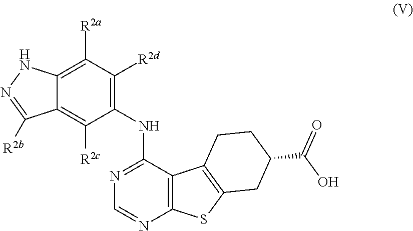

in which R^{2a}, R^{2b}, R^{2c}, and R^{2d} are as defined for the compounds of general formula (I), supra;
is allowed to react with a compound of general formula (VI):

R³R⁴—NH (VI)

in which R³ and R⁴ are as defined for the compounds of general formula (I), supra;
thus providing a compound of general formula (I):

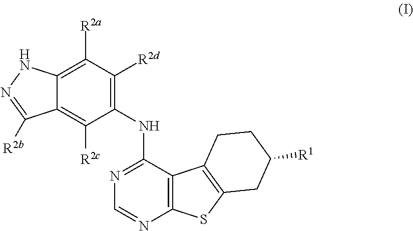

in which R¹, R^{2a}, R^{2b}, R^{2c}, and R^{2d} are as defined for the compounds of general formula (I), supra.

In accordance with a further aspect, the present invention covers intermediate compounds which are useful in the preparation of compounds of the present invention of general formula (I), particularly in the method described herein. In particular, the present invention covers compounds of general formula (VII):

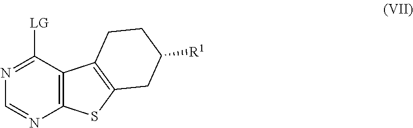

in which R¹ is as defined for the compounds of general formula (I), supra, and LG represents a leaving group.

In another preferred embodiment, the present invention covers intermediate compounds which are useful in the preparation of compounds of the present invention of general formula (I), particularly in the method described herein. In particular, the present invention covers compounds of general formula (V):

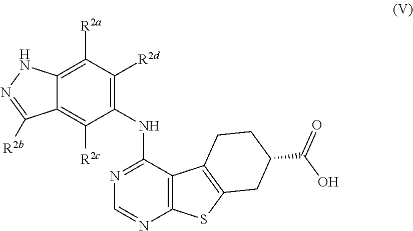

in which R^{2a}, R^{2b}, R^{2c}, and R^{2d} are as defined for the compounds of general formula (I), supra.

In accordance with yet another aspect, the present invention covers the use of the intermediate compounds of general formula (VII):

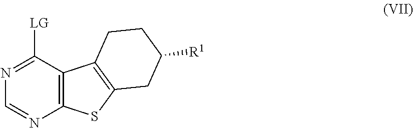

in which R¹ is as defined for the compounds of general formula (I), supra, and LG represents a leaving group;
for the preparation of a compound of general formula (I) as defined supra.

In another preferred embodiment, the present invention covers the use of the intermediate compounds of general formula (V):

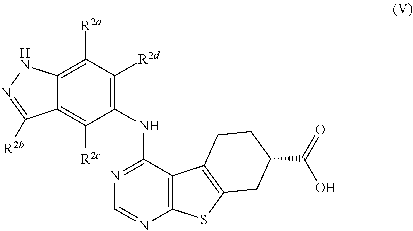

in which R^{2a}, R^{2b}, R^{2c}, and R^{2d} are as defined for the compounds of general formula (I), supra;
for the preparation of a compound of general formula (I) as defined supra.

### Synthesis of Compounds of General Formula (I) of the Present Invention

Compounds of general formula (I), wherein R¹, R^{2a}, R^{2b}, R^{2c}, and R^{2d} have the meaning as given for general formula (I), supra, can be synthesized according to a general procedure depicted in Scheme 1 starting from synthons of the formulae (II) and (III), wherein LG stands for a leaving group, and wherein RE represents a C₁-C₆-alkyl group.

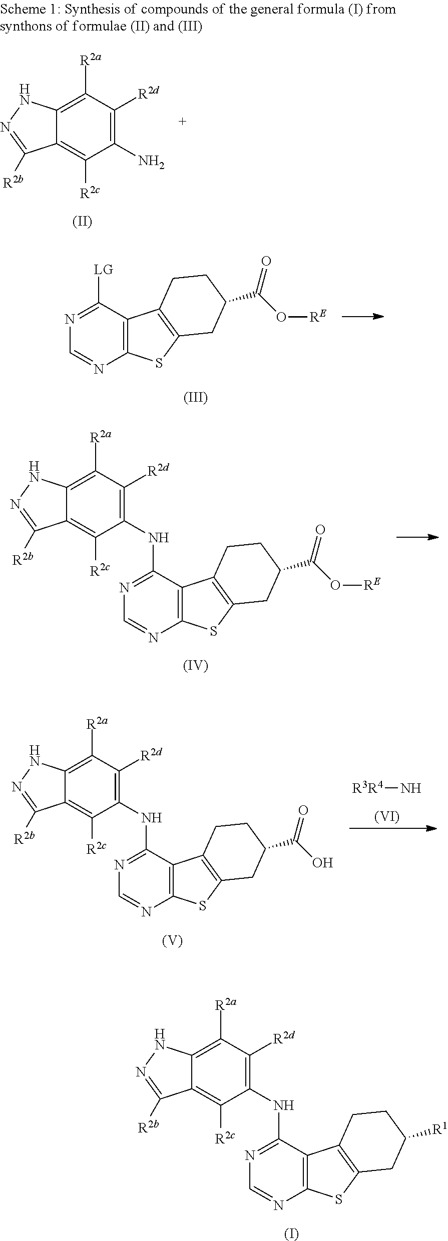

The route exemplified in Scheme 1 allows variations in R¹, R^{2a}, R^{2b}, R^{2c}, and R^{2d}, but is particularly suitable for R¹ diversification on the last synthetic step. The coupling of 5-aminoindazole derivatives of the formula (II) with enantiopure, pyrimidine-derived synthons such as (III) can be accomplished by reacting the two reactants in a suitable solvent, such as ethanol or a related lower aliphatic alcohol of the formula C₁-C₄-alkyl-OH or a cyclic ether, such as tetrahydrofuran or 1,4-dioxane, optionally in the presence of an acid such as hydrochloric acid. The 5-aminoindazole derivatives can be used either as free base or as corresponding salt with organic or inorganic acids. Alternatively, such amination reactions can be performed using catalysis by metals, such as palladium (see e.g. J. Y. Yoon et al., Synthesis 2009, (5), 815, and literature cited therein), to give compounds of formula (IV). The ester group present in compounds of formula (IV) can subsequently be hydrolysed to give the corresponding carboxylic acids of formula (V) by methods well known to the person skilled in the art, using an aqueous solution of an alkali hydroxide, preferably lithium hydroxide, in a suitable solvent aqueous aliphatic alcohol of the formula C₁-C₄-alkyl-OH, optionally containing a cyclic ether such as tetrahydrofuran as co-solvent. Said carboxylic acids of formula (V) can be coupled with amines of formula (VI), in which R³ and R⁴ have the meaning as given for general formula (I) and which are widely commercially available, with a suitable coupling agent, such as HATU, TBTU, or 2,4,6-tripropyl-1,3,5,2,4,6-trioxatriphosphinane 2,4,6-trioxide (also known as T3P), to give compounds of the general formula (I). Amines of formula (VI) can be used either as free base or as corresponding salt with organic or inorganic acids.

Modification of any of the substituents, R¹, R^{2a}, R^{2b}, R^{2c}, and R^{2d} can be achieved before and/or after the exemplified transformation. However, also other routes may be used to synthesise the target compounds, in accordance with common general knowledge of a person skilled in the art of organic synthesis.

Said modifications can be such as the introduction of protecting groups, cleavage of protecting groups, reduction or oxidation of functional groups, formation or cleavage of esters or carboxamides, halogenation, metallation, substitution or other reactions known to a person skilled in the art. These transformations include those which introduce a functionality which allows for further interconversion of substituents. Appropriate protecting groups and their introduction and cleavage are well-known to a person skilled in the art (see for example T. W. Greene and P. G. M. Wuts in Protective Groups in Organic Synthesis, 3^{rd} edition, Wiley 1999). Further, it is possible that two or more successive steps may be performed without work-up being performed between said steps, e.g. a “one-pot” reaction, as it is well-known to a person skilled in the art.

Vice versa, compounds of general formula (I), wherein R¹, R^{2a}, R^{2b}, R^{2c}, and R^{2d} have the meaning as given for general formula (I), supra, can be synthesized according to a complementary general procedure depicted in Scheme 2 starting from synthons of the formulae (II) and (VII), wherein LG stands for a leaving group.

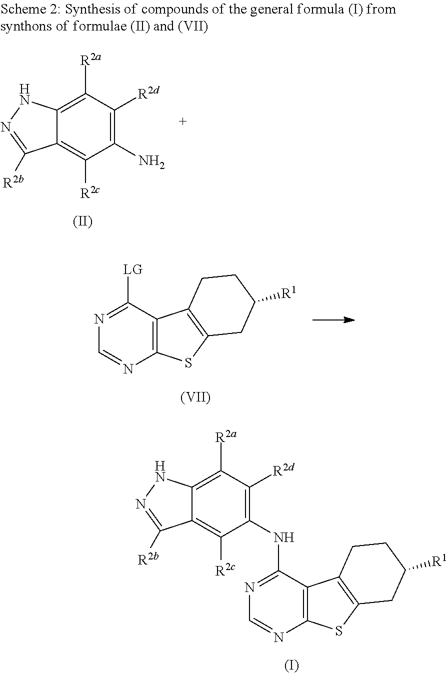

In the approach depicted in Scheme 2, an enantiopure pyrimidine based synthon of the formula (VII), already featuring the carboxamide moiety R¹ in place, is reacted with 5-aminoindazole derivatives of the formula (II) in an analogous fashion as described for the reaction between (II) and (III) in Scheme 1, supra.

Said enantiopure pyrimidine based synthons of the formulae (III) and (VII), in which R¹ is as defined for the general formula (I), RE represents a C₁-C₆-alkyl group and in which LG represents a leaving group as defined supra, can be prepared in accordance to the Schemes 3, 4 and 5 shown below.

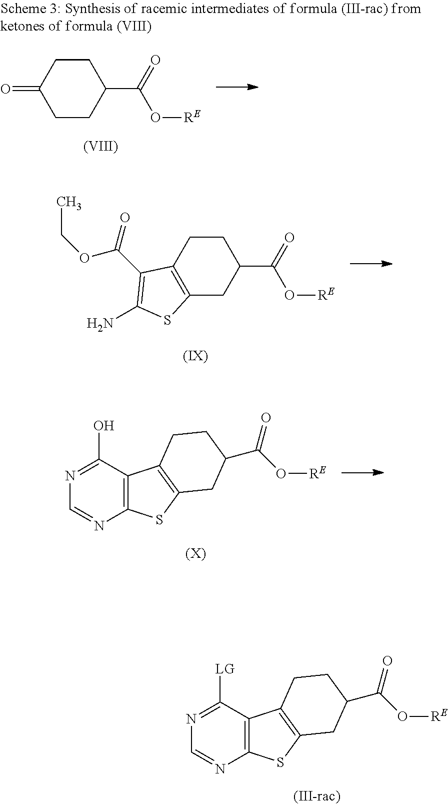

Racemic intermediate compounds of the general formula (III-rac), wherein RE represents a C₁-C₆-alkyl group, and wherein LG stands for a leaving group, are known to the person skilled in the art and can be readily prepared as shown in Scheme 3 by a so-called Gewald thiophene synthesis (for a seminal publication see e.g. K. Gewald et al., Chem. Ber. 1966, 94, 99), starting from ketones of the general formula (VIII), to give the intermediate thiophene derivatives (IX). Said intermediates are then cyclised to the hydroxy-thienopyrimidines (X), which are in equilibrium with their respective pyrimidone tautomers, employing a suitable C₁ synthon such as formamide. The resulting hydroxypyrimidines (X) are then transferred into compounds of the general formula (III-rac), in which LG represents a leaving group like, for example, a halogen atom as, for example, a chlorine or bromine atom, by suitable procedures known to the person skilled in the art, such as treatment by reacting the alcohol with a halogenation agent like, for example, phosphorus trichloride, phosphorus tribromide, phosphoric trichloride or phosphoric tribromide, preferably phosphoric trichloride (also named phosphorus oxychloride), with or without an additional inert solvent as, for example, toluene at temperatures ranging from room temperature to the boiling point of the solvent, for example. Compounds of formula (III-rac) in which LG represents a leaving group like, for example, an alkylsulfonate as, for example, methanesulfonate or trifluoromethanesulfonate or 1,1,2,2,3,3,4,4,4-nonafluorobutane-1-sulfonate or an arylsulfonate like, for example, benzenesulfonate or 4-methylbenzenesulfonate can be synthesised from compounds of formula (X), by reacting the alcohol with a suitable alkylsulfonyl halide as, for example, methanesulfonyl chloride or trifluoromethanesulfonyl chloride or 1,1,2,2,3,3,4,4,4-nonafluorobutane-1-sulfonyl fluoride or by reacting the alcohol with a suitable arylsulfonyl halide as, for example, benzenesulfonyl chloride or 4-methylbenzenesulfonyl chloride in an inert solvent like, for example, tetrahydrofuran or toluene or dichloromethane optionally in the presence of a suitable base like, for example, triethylamine or pyridine or N,N-dimethylpyridin-4-amine at temperatures ranging from −40° C. to the boiling point of the solvent, for example. An instructive exemplary protocol for the sequence outlined in Scheme 2 can be found in WO 2005/010008, example 14, steps 1 to 3.

Multiple methods of isolating pure enantiomers from isomeric mixtures, e.g. racemic mixtures of chiral compounds are known to the person skilled in the art. Said methods encompass preparative HPLC on chiral stationary phase, kinetic resolution of racemic mixtures (for some examples see e.g. I. Shiina et al., Catal. Sci Technol. 2012, 2, 2200-2205; I. Shiina et al., Eur. J. Org. Chem. 2008, 5887-5890; D. G. Walker et al., Organic Process Research [symbol] Development 2001, 5, 23-27; B. N. Roy et al., Organic Process Research [symbol] Development 2009, 13, 450; T. Storz and P. Dittmar, Organic Process Research [symbol] Development 2003, 7, 559), enantioselective protonation (for some examples see e.g. C. Fehr and G. Galindo, Helv. Chinn. Acta 1995, 78, 539-552, S. Hünig et al., Chem. Ber. 1994, 127, 1981-1988; S. Hünig et al., Chem. Ber. 1994, 127, 1969), enzymatic resolution (for some examples see e.g. T. Miyazawa, Amino Acids 1999, 16, 191-213), or, preferably and outlined in more detail below, temporary derivatisation with an enantiopure chiral synthon, separation of the resulting diastereomers, and removal of said chiral synthon, resulting in the isolation of the pure enantiomers of the parent compound (for some examples see e.g. Asymmetric Synthesis—The Essentials. Edited by Mathias Christmann and Stefan Brase, WILEY-VCH Verlag GmbH [symbol] Co. KGaA, Weinheim).

Scheme 4 illustrates the transformation of racemic pyrimidine synthons of the formula (IIIa-rac), in which RE represents a C₁-C₆-alkyl group, and in which Y stands for a leaving group LG or a hydroxyl group (being hence equivalent to a compound either of formula (X) or (III-rac) as outlined in Scheme 3), into an activated form such as an acid chloride of the formula (XII). It is well possible to hydrolyse the ester group present in said synthons (IIIa-rac) in the presence of Y e.g. representing a group LG e.g. representing a chloride, by mild ester hydrolysis using e.g. lithium hydroxide, as known by the person skilled in the art, to give carboxylic acids of formula (XI-rac). These can be readily converted into acid chlorides of the formula (XII) by methods well known to the person skilled in the art, such as the reaction with an inorganic acid chloride such as thionyl chloride.

Said acid chlorides (XII) are subsequently reacted with a chiral, enantiomerically pure synthon such as an oxazolidinone of the formula (XIII), in which R^{Ox1} represents a hydrogen atom or a C₁-C₄-alkyl group, preferably methyl, and in which R^{Ox2} represents an aryl, aryl-(CH₂)ₚ— or a C₁-C₄-alkyl- group, preferably phenyl, after deprotonation of said oxazolidinone using a suitable deprotonation agent such as n-butyllithium or sodium hydride, at temperatures ranging from −78° C. to 0° C., preferably below −40° C., to give the amide coupling product of formula (XIV) as mixture of two diastereomers. Said diastereomeric mixture can then be separated into the pure stereoisomers of formulae (XIVa) and (XIVb) using methods known to the person skilled in the art, such as fractionised crystallisation or column chromatography on silica gel.

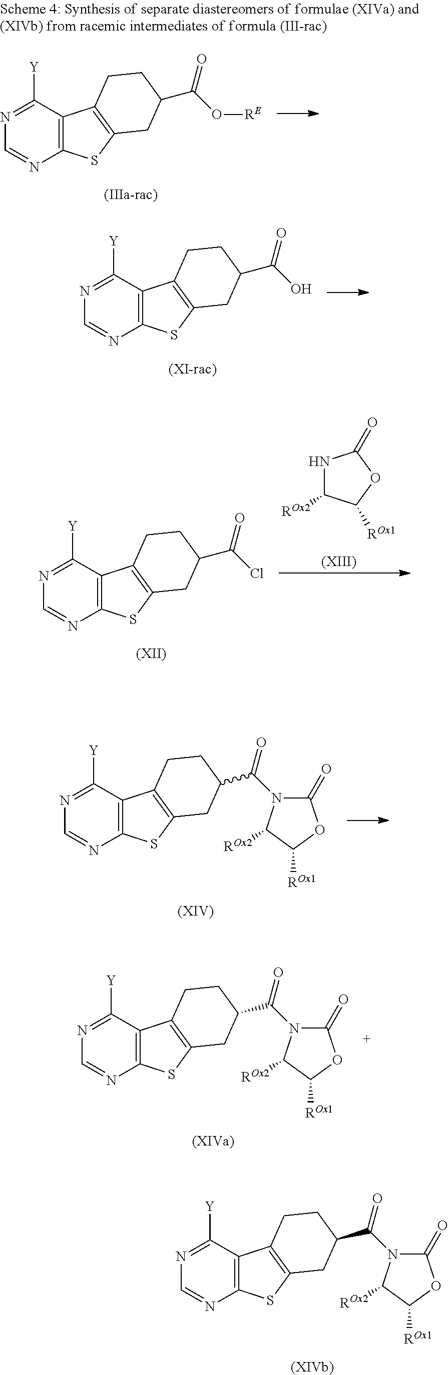

Scheme 5 illustrates the transformation of the desired stereoisomer (XIVa) to compounds of formula (VIIa), in which R^{E} represents a C₁-C₆-alkyl group, and in which Y stands for a leaving group LG or a hydroxyl group.

The desired stereoisomer (XIVa) can subsequently be converted into the enantiomerically pure ester synthons of formula (III), which can be further transformed into the compounds of the present invention as outlined in Scheme 1. Said transformation can be accomplished by various ways known to the person skilled in the art; preferably, intermediates of the formula (XIVa) are subjected to a transesterification reaction using, for example, titanium(IV)tetraethanolate in ethanol preferentially at elevated temperature. The resulting pyrimidine based ester synthons of formula (IIIa) can subsequently be subjected to mild hydrolysis, as discussed supra, to give enantiopure carboxylic acids of formula (XI), which in turn can be coupled with amines of formula (VI), in which R³ and R⁴ have the meaning as given for general formula (I) and which are widely commercially available, with a suitable coupling agent, such as HATU, TBTU, or 2,4,6-tripropyl-1,3,5,2,4,6-trioxatriphosphinane 2,4,6-trioxide (also known as T3P), to give enantiopure pyrimidine synthons of formula (VIIa), which can be further elaborated to give compounds of the general formula (I) as outlined in Scheme 2. If needed, compounds of formulae (IIIa) and (VIIa), in which Y represents a hydroxy group are converted into the respective compounds in which Y stands for a leaving group LG, i.e. into compounds of formulae (III) and (VII) referred to in Schemes 1 and 2, by the methods described supra.

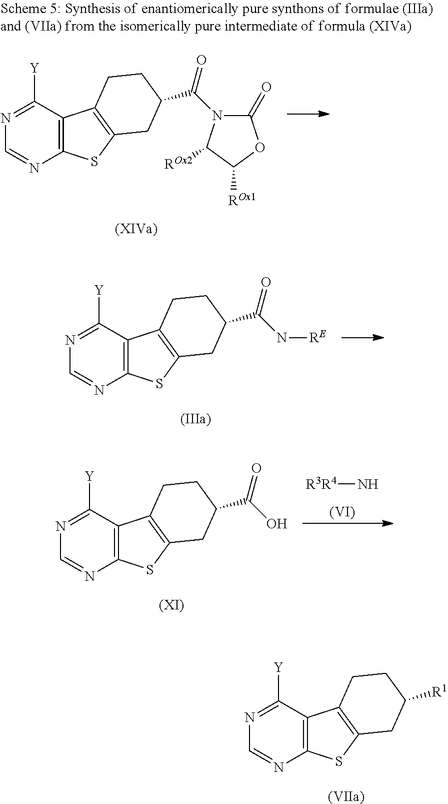

5-Aminoindazole synthons of the formula (II), in which R^{2a}, R^{2b}, R^{2c} and R^{2d} have the meaning as given for general formula (I), are known to the person skilled in the art, and are commercially available with a wide range of substituents. Their synthesis has been described inter alia by means of diazotation of the corresponding ortho-toluidines, followed by cyclisation to the indazole (see e.g. H. D. Porter and W. D. Peterson, Org. Syn., Coll. Vol. 3 (1955), 660, or U.S. Pat. No. 5,444,038). Recently, the synthesis of substituted indazoles suitable as intermediates via reaction of ortho-fluorobenzaldehydes with hydrazine hydrate has been described (see e.g. R. C. Wheeler et al., Org. Process Res. Dev 2011, 15, 565, for a related publication see also K. Lukin et al., J. Org. Chem. 2006, 71, 8166). Both processes typically yield indazoles featuring an amine precursor, such as a nitro group, which can be readily converted into the desired indazole-5-amine by reduction (see e.g. J. Med. Chem. 2003, 46, 5663).

As an example of said synthetic approach, Scheme 6 outlines the preparation of Indazole intermediates of the formula (IIa), in which R^{2a}, R^{2b} and R^{2c} have the meaning as given for general formula (I), and in which R^{2d} represents —OR⁵, which constitute a subset of the compounds of formula (II). Nitration of 2-fluoro-4-hydroxybenzaldehyde (XV, CAS-No. 348-27-6) using nitric acid in sulfuric acid gives rise to the corresponding 5-nitro-benzaldehyde (XVI), which can be subsequently cyclised e.g. by reacting with hydrazine hydrate in a suitable solvent, such as an aliphatic alcohol of the formula C₁-C₄-alkyl-OH, e.g. ethanol, preferentially at elevated temperature. The resulting 5-nitro-1H-indazol-6-ol (XVII) can be chemoselectively O-alkylated by a so-called Mitsunobu reaction (Mitsunobu, O. Synthesis 1981, 1, 1-28) with an aliphatic alcohol R⁵—OH, a suitable trialkyl or triaryl phosphane, such as triphenylphosphane, and a suitable dialkyl azodicarboxylate, such as diisopropyl azodicarboxylate, in a suitable solvent, such as a cyclic ether, e.g. tetrahydrofuran, resulting in 6-alkoxy-5-nitro-(1H)-indazoles of the formula (XVIII), which can be reduced to the corresponding 6-aminoindazole derivatives of formula (IIa) by reduction methods well known to the person skilled in the art, e.g. by reaction with a palladium/charcoal catalyst in a suitable solvent such as methanol, ethanol, or tetrahydrofuran.

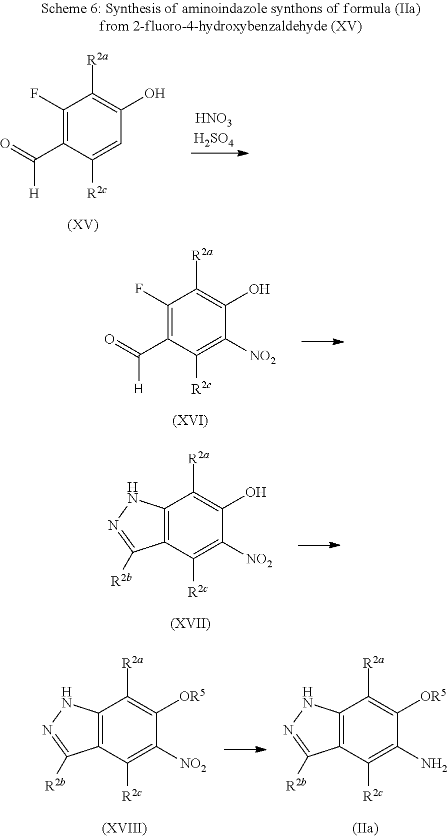

In an analogous fashion, as outlined in Scheme 7, 6-amino indazole derivatives of formula (IIb), in which R^{2a}, R^{2b} and R^{2c} have the meaning as given for general formula (I), and in which R^{2d} represents —N(R⁶)R⁷, constituting another subset of the compounds of formula (II), can be prepared from 2-fluoro-4-trifluoromethoxybenzaldehyde (XIX, CAS-No. 1227628-83-2), by nitration with nitric acid and sulfuric acid to give the corresponding nitrobenzaldehyde (XX). In a similar way as shown above in Scheme 6, (XX) can be subsequently cyclised e.g. by reacting with hydrazine hydrate in a suitable solvent, such as N,N-dimethyl acetamide, preferentially at elevated temperature, to give 5-nitro-6-trifluoromethoxy-1H-indazole (XXI). Nucleophilic displacement of the trifluoromethoxy group present in (XXI) by an amine of the formula (Via), in which R⁶ and R⁷ have the meaning as given for general formula (I), in dimethyl sulfoxide preferentially at elevated temperature, gives rise to substituted 6-amino-5-nitro indazole derivatives of formula (XXII), which in turn can be reduced as described supra to the corresponding diamino indazole derivatives of formula (IIb). Noteworthily, commercial 6-halo-5-nitro-1H-indazoles such as 6-chloro-5-nitro-1H-indazole (CAS-No. 101420-98-8) can be employed in a similar fashion as described supra for 5-nitro-6-trifluoromethoxy-1H-indazole (XXI).

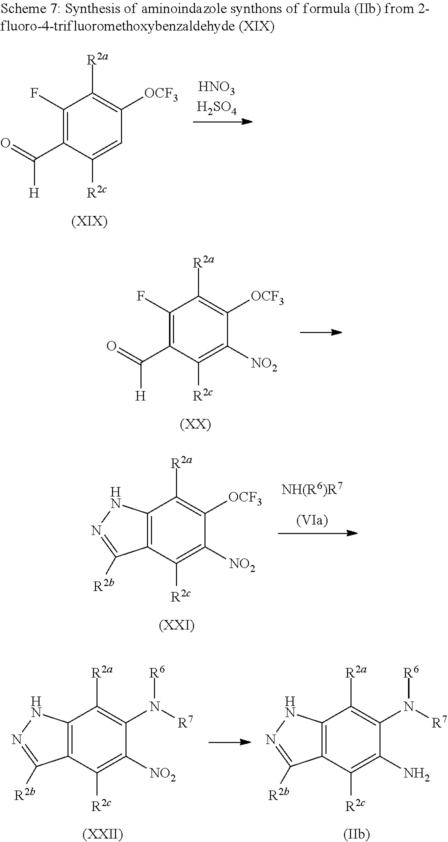

Replacement of the amine HN(R⁶)R⁷ by a thiol H—SR⁶, or an alkali salt thereof, in abovementioned nucleophilic displacement of the trifluoromethoxy group present in (XXI) allows for the preparation of 6-alkylthioindazole derivatives of formula (IIc) representing yet another subset of the compounds of the formula (II) as outlined in Scheme 8. Said amines HN(R⁶)R⁷, alike thiols H—SR⁶ and alkali salts thereof, are known to the person skilled in the art and are widely commercially available.

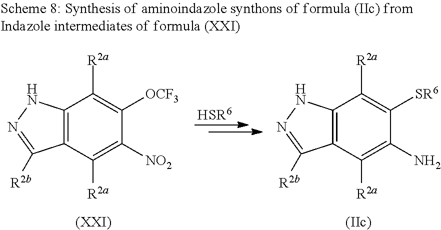

Optionally, the indazole intermediates of formulae (IIa), (IIb), (IIc), (XVII), (XVIII), (XXI) and (XXI I) may be transiently be protected at position 1 by a suitable protective group like, for example, a benzyl-, an allyl-, an allyloxycarbonyl- or an C₁-C₆-alkoxycarbonyl- group.

### EXPERIMENTAL SECTION

The following table lists the abbreviations used in this paragraph, and in the examples section.

| Abbreviation | Meaning |
|---|---|
| HPLC | high performance liquid chromatography |
| LC-MS | liquid chromatography - mass spectrometry |
| NMR | nuclear magnetic resonance |
| DMSO | dimethylsulfoxide |
| ppm | parts per million |

### EXAMPLES

Chemical naming of the examples and intermediates was performed using ACD software by ACD/LABS (Batch version 12.01.)

### Example 1

### (7S)-4-(1H-Indazol-5-ylamino)-N,N-dimethyl-5,6,7,8-tetrahydro[1]benzothieno[2,3-d]pyrimidine-7-carboxamide

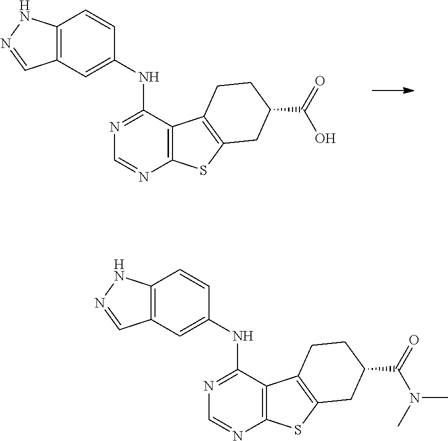 [SMILES: O=C(O)[C@H]1CCc2c(sc3ncnc(Nc4ccc5[nH]ncc5c4)c23)C1 ; CN(C)C(=O)[C@H]1CCc2c(sc3ncnc(Nc4ccc5[nH]ncc5c4)c23)C1]

A mixture comprising 191.5 mg (524 μmol) (7S)-4-(1H-indazol-5-ylamino)-5,6,7,8-tetrahydro[1]benzothieno[2,3-d]pyrimidine-7-carboxylic acid (prepared according to intermediate example 1a), 5.84 mL N,N-dimethylformamide, 548 μL N-ethyl-N-isopropylpropan-2-amine, 1.57 mL N-methylmethanamine (2M solution in tetrahydrofurane) and 624 μL 2,4,6-tripropyl-1,3,5,2,4,6-trioxatriphosphinane 2,4,6-trioxide solution (50% in N,N-dimethylformamide) was stirred at 23° C. overnight. The solvents were removed and the residue purified by chromatography to give 56.3 mg (26%) of the title compound.

¹H-NMR (DMSO-d6): δ=1.77 (1H), 2.06 (1H), 2.87 (3H), 2.92 (2H), 3.09 (3H), 3.11-3.34 (3H), 7.46-7.53 (2H), 7.98 (1H), 8.04 (1H), 8.18 (1H), 8.30 (1H), 13.00 (1H) ppm.

### Example 1a

### (7S)-4-(1H-Indazol-5-ylamino)-5,6,7,8-tetrahydro[1]benzothieno[2,3-d]pyrimidine-7-carboxylic acid

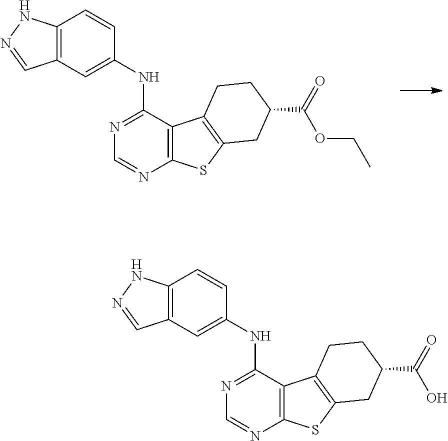 [SMILES: CCOC(=O)[C@H]1CCc2c(sc3ncnc(Nc4ccc5[nH]ncc5c4)c23)C1 ; O=C(O)[C@H]1CCc2c(sc3ncnc(Nc4ccc5[nH]ncc5c4)c23)C1]

A mixture comprising 3.64 g (9.25 mmol) ethyl (7S)-4-(1H-indazol-5-ylamino)-5,6,7,8-tetrahydro[1]benzothieno[2,3-d]pyrimidine-7-carboxylate (prepared according to intermediate example 1b), 160 mL tetrahydrofurane, 40 mL methanol and 55.5 mL lithium hydroxide solution (1M in water) was stirred at 23° C. for three hours. The mixture was acidified with hydrochloric acid, 100 mL dichloromethane were added, the precipitate was filtered off, washed with water and dried to give 3.33 g (90%) of the title compound.

### Example 1b

### Ethyl (7S)-4-(1H-indazol-5-ylamino)-5,6,7,8-tetrahydro[1]benzothieno[2,3-d]pyrimidine-7-carboxylate

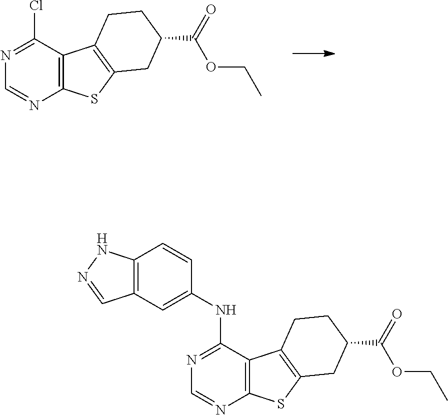 [SMILES: CCOC(=O)[C@H]1CCc2c(sc3ncnc(Cl)c23)C1 ; CCOC(=O)[C@H]1CCc2c(sc3ncnc(Nc4ccc5[nH]ncc5c4)c23)C1]

A mixture comprising 5.00 g (16.85 mmol) ethyl (7S)-4-chloro-5,6,7,8-tetrahydro[1]benzothieno[2,3-d]pyrimidine-7-carboxylate (prepared according to intermediate example 1c), 2.13 g 1H-indazol-5-amine and 125 mL ethanol was heated at reflux overnight. 1.0 mL triethylamine were added and the solvents removed. The residue was solved in hot methanol, dichloromethane was added and the mixture was stirred at 23° C. The formed precipitate was collected to give 5.06 g (76%) of the title compound.

### Example 1c

### Ethyl (7S)-4-chloro-5,6,7,8-tetrahydro[1]benzothieno[2,3-d]pyrimidine-7-carboxylate

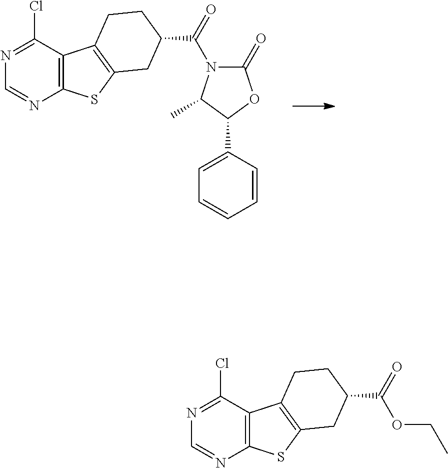 [SMILES: C[C@H]1[C@@H](c2ccccc2)OC(=O)N1C(=O)[C@H]1CCc2c(sc3ncnc(Cl)c23)C1 ; CCOC(=O)[C@H]1CCc2c(sc3ncnc(Cl)c23)C1]

A mixture comprising 27.6 g (64.6 mmol) (4S,5R)-3-{[(7S)-4-chloro-5,6,7,8-tetrahydro[1]benzothieno[2,3-d]pyrimidin-7-yl]carbonyl}-4-methyl-5-phenyl-1,3-oxazolidin-2-one (prepared according to intermediate example 1d), 830 mL ethanol and 24.4 mL titanium (4+) tetraethanolate was refluxed for 20 hours. 1.4 L ethyl acetate and 18 mL water were added and the mixture was stirred for 30 minutes. Silica gel was added and stirring was continued for 10 minutes. The mixture was filtered through celite, the solvents were removed and the residue was purified by chromatography to give 18.8 g (93%) of the title compound.

### Example 1d

### (4S,5R)-3-{[(7S)-4-Chloro-5,6,7,8-tetrahydro[1]benzothieno[2,3-d]pyrimidin-7-yl]carbonyl}-4-methyl-5-phenyl-1,3-oxazolidin-2-one (A) and (4S,5R)-3-{[(7R)-4-chloro-5,6,7,8-tetrahydro[1]benzothieno[2,3-d]pyrimidin-7-yl]carbonyl}-4-methyl-5-phenyl-1,3-oxazolidin-2-one (B)

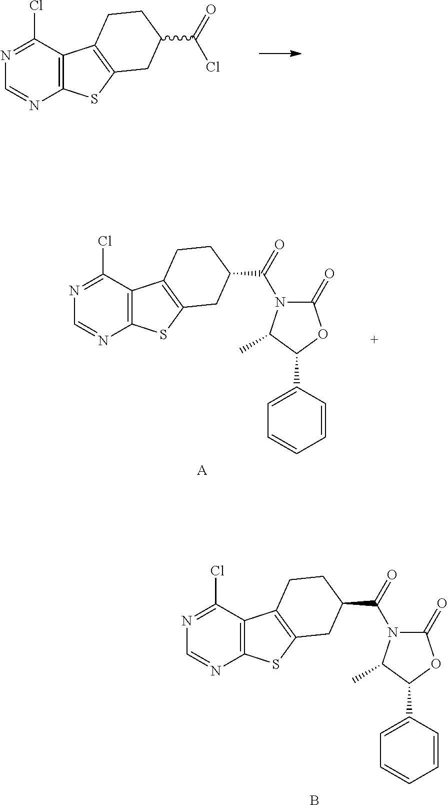

To a solution of 26.8 g (4S,5R)-4-methyl-5-phenyl-1,3-oxazolidin-2-one in 428 mL tetrahydrofurane were added 70 mL n-buthyllithium (2.5 M in hexane) at −78° C. and the mixture was stirred at −60° C. for 1 hour. A solution of 45.8 g (159 mmol) 4-chloro-5,6,7,8-tetrahydro[1]benzothieno[2,3-d]pyrimidine-7(RS)-carbonyl chloride (prepared according to intermediate example 1e) in 428 mL tetrahydrofurane was added and stirring was continued at −70° C. for 1 hour. The mixture was poured into water, tetrahydrofurane was removed, the precipitate was filtered off, washed with water and resolved in dichloromethane. The organic layer was dried over sodium sulphate followed by addition of acetonitrile. The dichloromethane was removed, the precipitate filtered, washed with acetonitrile and diethylether to give 27.6 g (38%) of the title compound A. From the mother liquor a second precipitate was obtained on standing overnight to give 25.5 g (35%) of the title compound B.

### Example 1e

### 4-Chloro-5,6,7,8-tetrahydro[1]benzothieno[2,3-d]pyrimidine-7(RS)-carbonyl chloride

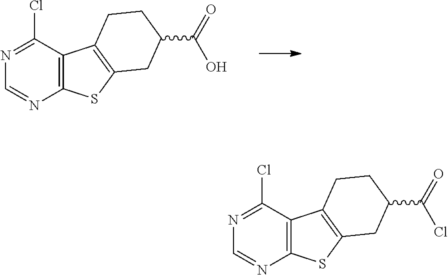 [SMILES: O=C(O)C1CCc2c(sc3ncnc(Cl)c23)C1 ; O=C(Cl)C1CCc2c(sc3ncnc(Cl)c23)C1]

A mixture comprising 42.87 g (159 mmol) 4-chloro-5,6,7,8-tetrahydro[1]benzothieno[2,3-d]pyrimidine-7-carboxylic acid (prepared according to intermediate example 1f) and 349 mL thionyl chloride was heated at 100° C. for 3 hours. The reagent was removed to give the title compound that was used without further purification.

### Example 1f

### (RS)-4-Chloro-5,6,7,8-tetrahydro[1]benzothieno[2,3-d]pyrimidine-7-carboxylic acid

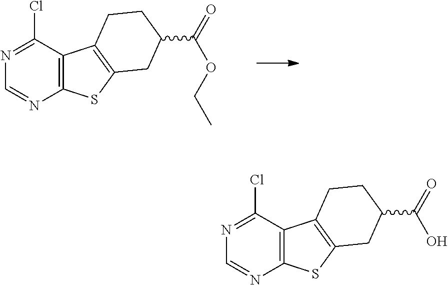 [SMILES: CCOC(=O)C1CCc2c(sc3ncnc(Cl)c23)C1 ; O=C(O)C1CCc2c(sc3ncnc(Cl)c23)C1]

20.0 g (37.4 mmol) (RS)-ethyl 4-chloro-5,6,7,8-tetrahydro[1]benzothieno[2,3-d]pyrimidine-7-carboxylate (prepared according to intermediate example 1g) were transformed in analogy to intermediate example 1a to give after working up and purification 17.2 g (95%) of the title compound.

### Example 1g

### (RS)-Ethyl 4-chloro-5,6,7,8-tetrahydro[1]benzothieno[2,3-d]pyrimidine-7-carboxylate

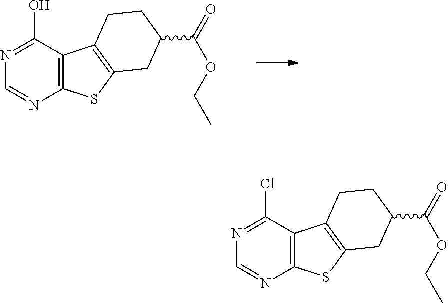 [SMILES: CCOC(=O)C1CCc2c(sc3ncnc(O)c23)C1 ; CCOC(=O)C1CCc2c(sc3ncnc(Cl)c23)C1]

A mixture comprising 195 g (700.6 mmol) (RS)-ethyl 4-hydroxy-5,6,7,8-tetrahydro[1]benzothieno[2,3-d]pyrimidine-7-carboxylate (prepared according to WO2005/10008), 1.92 L toluene, 195 mL N-ethyl-N-isopropylpropan-2-amine and 78.4 mL phosphorus oxychloride was heated at 80° C. overnight. The mixture was poured into sodium hydrogencarbonate solution and extracted with ethyl acetate. The organic layer was washed with brine and dried over sodium sulphate. After filtration and removal of the solvent the residue was crystallized from diisopropyl ether to give 120 g (58%) of the title compound.

### Example 2

### Reference Example

### (7R)-4-(1H-Indazol-5-ylamino)-N,N-dimethyl-5,6,7,8-tetrahydro[1]benzothieno[2,3-d]pyrimidine-7-carboxamide

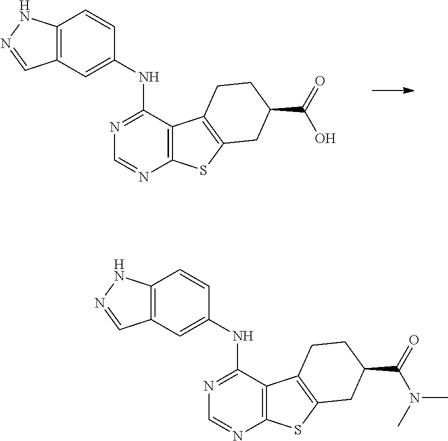 [SMILES: O=C(O)[C@@H]1CCc2c(sc3ncnc(Nc4ccc5[nH]ncc5c4)c23)C1 ; CN(C)C(=O)[C@@H]1CCc2c(sc3ncnc(Nc4ccc5[nH]ncc5c4)c23)C1]

192 mg (526 μmol) (7R)-4-(1H-indazol-5-ylamino)-5,6,7,8-tetrahydro[1]benzothieno[2,3-d]pyrimidine-7-carboxylic acid (prepared according to intermediate example 2a) were transformed in analogy to example 1 to give after working up and purification 99.2 mg (46%) of the title compound.

¹H-NMR (DMSO-d6): δ=1.77 (1H), 2.06 (1H), 2.87 (3H), 2.92 (2H), 3.09 (3H), 3.11-3.34 (3H), 7.46-7.53 (2H), 7.98 (1H), 8.04 (1H), 8.18 (1H), 8.30 (1H), 13.00 (1H) ppm.

### Example 2a

### (7R)-4-(1H-Indazol-5-ylamino)-5,6,7,8-tetrahydro[1]benzothieno[2,3-d]pyrimidine-7-carboxylic acid

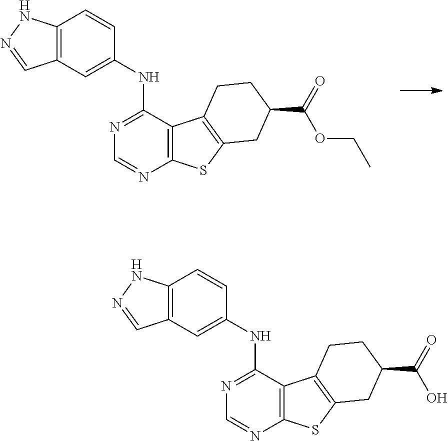 [SMILES: CCOC(=O)[C@@H]1CCc2c(sc3ncnc(Nc4ccc5[nH]ncc5c4)c23)C1 ; O=C(O)[C@@H]1CCc2c(sc3ncnc(Nc4ccc5[nH]ncc5c4)c23)C1]

260.4 mg (662 μmol) ethyl (7R)-4-(1H-indazol-5-ylamino)-5,6,7,8-tetrahydro[1]benzothieno[2,3-d]pyrimidine-7-carboxylate (prepared according to intermediate example 2b) were transformed in analogy to intermediate example 1a to give after working up and purification 201.7 mg (79%) of the title compound.

### Example 2b

### Ethyl (7R)-4-(1H-indazol-5-ylamino)-5,6,7,8-tetrahydro[1]benzothieno[2,3-d]pyrimidine-7-carboxylate

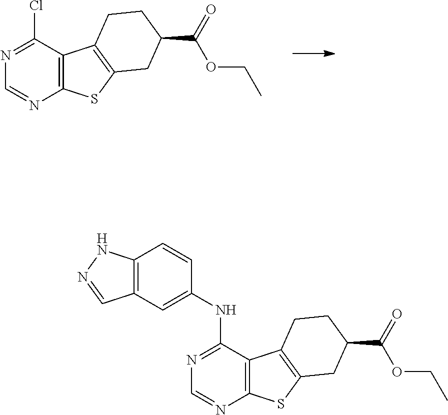 [SMILES: CCOC(=O)[C@@H]1CCc2c(sc3ncnc(Cl)c23)C1 ; CCOC(=O)[C@@H]1CCc2c(sc3ncnc(Nc4ccc5[nH]ncc5c4)c23)C1]

350 mg (1.18 mmol) ethyl (7R)-4-chloro-5,6,7,8-tetrahydro[1]benzothieno[2,3-d]pyrimidine-7-carboxylate (prepared according to intermediate example 2c) were transformed in analogy to intermediate example 1b to give after working up and purification 270.3 mg (55%) of the title compound.

### Example 2c

### Ethyl (7R)-4-chloro-5,6,7,8-tetrahydro[1]benzothieno[2,3-d]pyrimidine-7-carboxylate

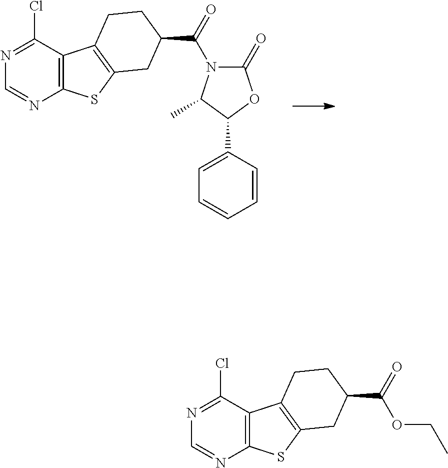 [SMILES: C[C@H]1[C@@H](c2ccccc2)OC(=O)N1C(=O)[C@@H]1CCc2c(sc3ncnc(Cl)c23)C1 ; CCOC(=O)[C@@H]1CCc2c(sc3ncnc(Cl)c23)C1]

10.2 g (23.84 mmol) (4S,5R)-3-{[(7R)-4-chloro-5,6,7,8-tetrahydro[1]benzothieno[2,3-d]pyrimidin-7-yl]carbonyl}-4-methyl-5-phenyl-1,3-oxazolidin-2-one (prepared according to intermediate example 1d) were transformed in analogy to intermediate example 1c to give after working up and purification 6.77 g (91%) of the title compound.

### Example 3

Azetidin-1-yl[(7S)-4-(1H-indazol-5-ylamino)-5,6,7,8-tetrahydro[1]benzothieno[2,3-d]pyrimidin-7-yl]methanone

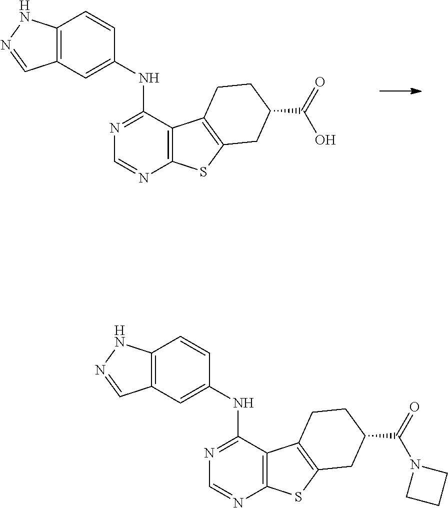 [SMILES: O=C(O)[C@H]1CCc2c(sc3ncnc(Nc4ccc5[nH]ncc5c4)c23)C1 ; O=C([C@H]1CCc2c(sc3ncnc(Nc4ccc5[nH]ncc5c4)c23)C1)N1CCC1]

91 mg (249 μmol) (7S)-4-(1H-indazol-5-ylamino)-5,6,7,8-tetrahydro[1]benzothieno[2,3-d]pyrimidine-7-carboxylic acid (prepared according to intermediate example 1a) were transformed in analogy to example 1 using azetidine to give after working up and purification 78.5 mg (74%) of the title compound.

¹H-NMR (DMSO-d6): δ=1.75 (1H), 2.04 (1H), 2.21 (2H), 2.72 (1H), 2.89 (2H), 3.16 (1H), 3.28 (1H), 3.88 (2H), 4.17-4.29 (2H), 7.46-7.53 (2H), 7.98 (1H), 8.04 (1H), 8.18 (1H), 8.30 (1H), 13.00 (1H) ppm.

### Example 4

### (7S)—N-Ethyl-4-(1H-indazol-5-ylamino)-N-methyl-5,6,7,8-tetrahydro[1]benzothieno[2,3-d]pyrimidine-7-carboxamide

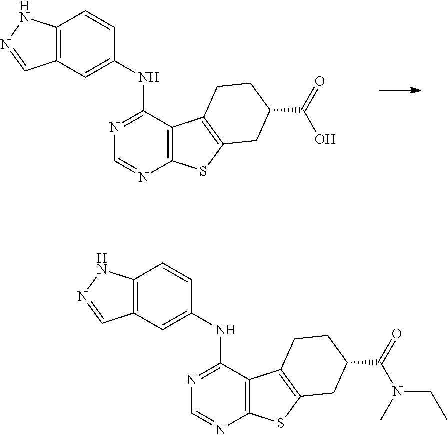 [SMILES: O=C(O)[C@H]1CCc2c(sc3ncnc(Nc4ccc5[nH]ncc5c4)c23)C1 ; CCN(C)C(=O)[C@H]1CCc2c(sc3ncnc(Nc4ccc5[nH]ncc5c4)c23)C1]

91 mg (249 μmol) (7S)-4-(1H-indazol-5-ylamino)-5,6,7,8-tetrahydro[1]benzothieno[2,3-d]pyrimidine-7-carboxylic acid (prepared according to intermediate example 1a) were transformed in analogy to example 1 using N-methylethanamine to give after working up and purification 40.7 mg (38%) of the title compound.

¹H-NMR (DMSO-d6): δ=1.02+1.14 (3H), 1.78 (1H), 2.04 (1H), 2.84+3.06 (3H), 2.86-3.50 (7H), 7.45-7.54 (2H), 7.98 (1H), 8.05 (1H), 8.19 (1H), 8.30 (1H), 13.02 (1H) ppm.

### Example 5

### (7S)-4-(1H-Indazol-5-ylamino)-N-methyl-N-(2, 2, 2-trifluoroethyl)-5,6,7,8-tetrahydro[1]benzothieno[2,3-d]pyrimidine-7-carboxamide

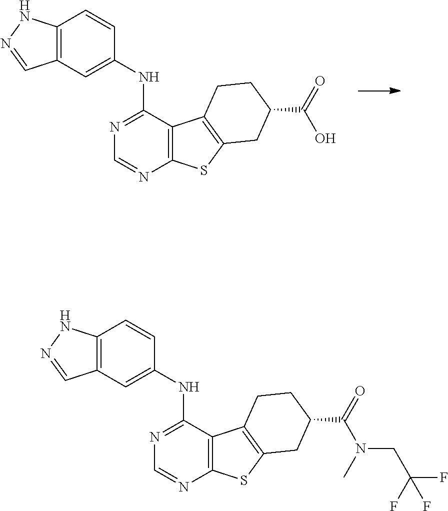 [SMILES: O=C(O)[C@H]1CCc2c(sc3ncnc(Nc4ccc5[nH]ncc5c4)c23)C1 ; CN(CC(F)(F)F)C(=O)[C@H]1CCc2c(sc3ncnc(Nc4ccc5[nH]ncc5c4)c23)C1]

91 mg (249 μmol) (7S)-4-(1H-indazol-5-ylamino)-5,6,7,8-tetrahydro[1]benzothieno[2,3-d]pyrimidine-7-carboxylic acid (prepared according to intermediate example 1a) were transformed in analogy to example 1 using 2,2,2-trifluoro-N-methylethanamine to give after working up and purification 67.6 mg (56%) of the title compound.

¹H-NMR (DMSO-d6): δ=1.80 (1H), 2.10 (1H), 2.97+3.22 (3H), 2.95 (2H), 3.15-3.29 (3H), 4.14-4.60 (2H), 7.45-7.54 (2H), 7.97 (1H), 8.05 (1H), 8.21 (1H), 8.30 (1H), 13.03 (1H) ppm.

### Example 6

### (7S)-4-(1H-Indazol-5-ylamino)-N-methyl-N-(3,3,3-trifluoropropyl)-5,6,7,8-tetrahydro[1]benzothieno[2,3-d]pyrimidine-7-carboxamide

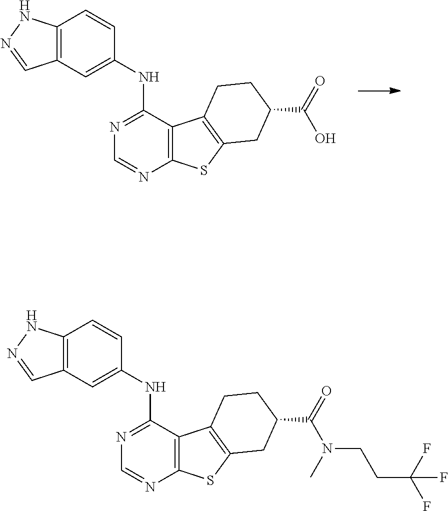 [SMILES: O=C(O)[C@H]1CCc2c(sc3ncnc(Nc4ccc5[nH]ncc5c4)c23)C1 ; CN(CCC(F)(F)F)C(=O)[C@H]1CCc2c(sc3ncnc(Nc4ccc5[nH]ncc5c4)c23)C1]

91 mg (249 μmol) (7S)-4-(1H-indazol-5-ylamino)-5,6,7,8-tetrahydro[1]benzothieno[2,3-d]pyrimidine-7-carboxylic acid (prepared according to intermediate example 1a) were transformed in analogy to example 1 using 3,3,3-trifluoro-N-methylpropan-1-amine to give after working up and purification 91.4 mg (74%) of the title compound.

¹H-NMR (DMSO-d6): δ=1.78 (1H), 2.05 (1H), 2.45-2.75 (2H), 2.87+3.11 (3H), 2.93 (2H), 3.04-3.30 (3H), 3.46-3.70 (2H), 7.46-7.53 (2H), 7.98 (1H), 8.04 (1H), 8.19 (1H), 8.30 (1H), 13.01 (1H) ppm.

### Example 7

### (7S)-4-(1H-Indazol-5-ylamino)-N-methyl-N-(propan-2-yl)-5,6,7,8-tetrahydro[1]benzothieno[2,3-d]pyrimidine-7-carboxamide

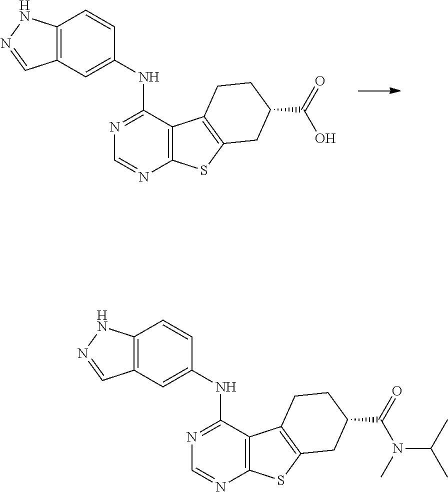 [SMILES: O=C(O)[C@H]1CCc2c(sc3ncnc(Nc4ccc5[nH]ncc5c4)c23)C1 ; CC(C)N(C)C(=O)[C@H]1CCc2c(sc3ncnc(Nc4ccc5[nH]ncc5c4)c23)C1]

91 mg (249 μmol) (7S)-4-(1H-indazol-5-ylamino)-5,6,7,8-tetrahydro[1]benzothieno[2,3-d]pyrimidine-7-carboxylic acid (prepared according to intermediate example 1a) were transformed in analogy to example 1 using N-methylpropan-2-amine to give after working up and purification 63.4 mg (58%) of the title compound.

¹H-NMR (DMSO-d6): δ=1.05+1.18 (6H), 1.80 (1H), 2.03 (1H), 2.70+2.90 (3H), 2.83-3.30 (5H), 4.26+4.72 (1H), 7.46-7.53 (2H), 7.98 (1H), 8.04 (1H), 8.17 (1H), 8.30 (1H), 13.01 (1H) ppm.

### Example 8

### (7S)—N-Ethyl-4-(1H-indazol-5-ylamino)-N-(propan-2-yl)-5,6,7,8-tetrahydro[1]benzothieno[2,3-d]pyrimidine-7-carboxamide

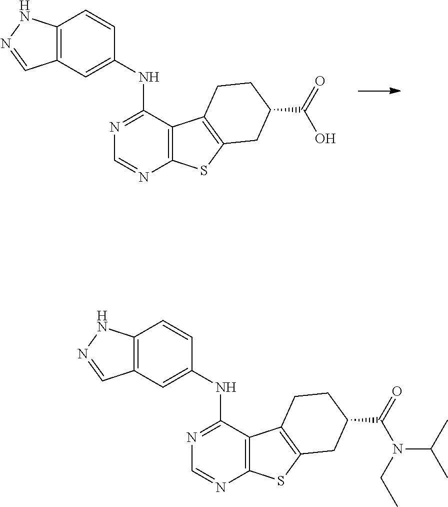 [SMILES: O=C(O)[C@H]1CCc2c(sc3ncnc(Nc4ccc5[nH]ncc5c4)c23)C1 ; CCN(C(=O)[C@H]1CCc2c(sc3ncnc(Nc4ccc5[nH]ncc5c4)c23)C1)C(C)C]

91 mg (249 μmol) (7S)-4-(1H-indazol-5-ylamino)-5,6,7,8-tetrahydro[1]benzothieno[2,3-d]pyrimidine-7-carboxylic acid (prepared according to intermediate example 1a) were transformed in analogy to example 1 using N-ethylpropan-2-amine to give after working up and purification 27.1 mg (24%) of the title compound.

¹H-NMR (DMSO-d6): δ=1.02-1.23 (9H), 1.82 (1H), 2.01 (1H), 2.81-3.30 (7H), 4.22+4.56 (1H), 7.45-7.54 (2H), 7.98 (1H), 8.05 (1H), 8.18 (1H), 8.30 (1H), 13.01 (1H) ppm.

### Example 9

### [(7S)-4-(1H-Indazol-5-ylamino)-5,6,7,8-tetrahydro[1]benzothieno[2,3-d]pyrimidin-7-yl](morpholin-4-yl)methanone

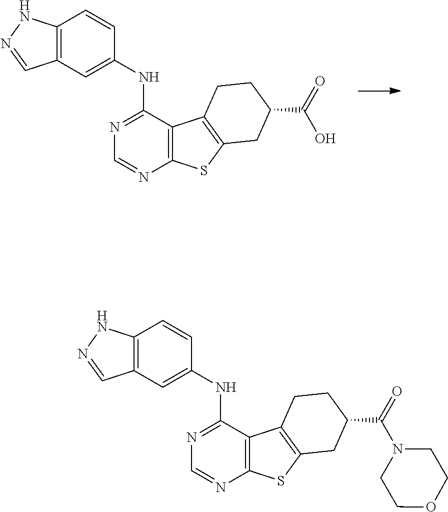 [SMILES: O=C(O)[C@H]1CCc2c(sc3ncnc(Nc4ccc5[nH]ncc5c4)c23)C1 ; O=C([C@H]1CCc2c(sc3ncnc(Nc4ccc5[nH]ncc5c4)c23)C1)N1CCOCC1]

91 mg (249 μmol) (7S)-4-(1H-indazol-5-ylamino)-5,6,7,8-tetrahydro[1]benzothieno[2,3-d]pyrimidine-7-carboxylic acid (prepared according to intermediate example 1a) were transformed in analogy to example 1 using morpholine to give after working up and purification 27.0 mg (24%) of the title compound.

¹H-NMR (DMSO-d6): δ=1.80 (1H), 2.05 (1H), 2.86-3.02 (2H), 3.13-3.30 (3H), 3.43-3.65 (8H), 7.46-7.53 (2H), 7.98 (1H), 8.04 (1H), 8.18 (1H), 8.30 (1H), 13.01 (1H) ppm.

### Example 10

### [(7S)-4-(1H-Indazol-5-ylamino)-5,6,7,8-tetrahydro[1]benzothieno[2,3-d]pyrimidin-7-yl][(1S,4S)-2-oxa-5-azabicyclo[2.2.1]hept-5-yl]methanone

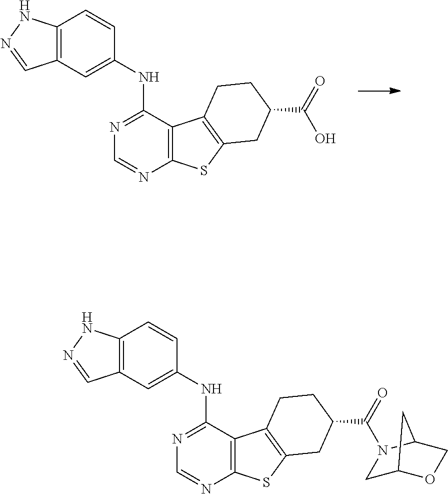 [SMILES: O=C(O)[C@H]1CCc2c(sc3ncnc(Nc4ccc5[nH]ncc5c4)c23)C1 ; O=C([C@H]1CCc2c(sc3ncnc(Nc4ccc5[nH]ncc5c4)c23)C1)N1CC2CC1CO2]

91 mg (249 μmol) (7S)-4-(1H-indazol-5-ylamino)-5,6,7,8-tetrahydro[1]benzothieno[2,3-d]pyrimidine-7-carboxylic acid (prepared according to intermediate example 1a) were transformed in analogy to example 1 using (1S,4S)-2-oxa-5-azabicyclo[2.2.1]heptane to give after working up and purification 64.7 mg (55%) of the title compound.

¹H-NMR (DMSO-d6): δ=1.74-1.89 (3H), 2.05 (1H), 2.78-3.38 (6H), 3.54 (1H), 3.63-3.78 (2H), 4.60+4.65 (1H), 4.76+4.86 (1H), 7.46-7.53 (2H), 7.98 (1H), 8.04 (1H), 8.18 (1H), 8.30 (1H), 13.01 (1H) ppm.

### Example 11

### [(7S)-4-(1H-Indazol-5-ylamino)-5,6,7,8-tetrahydro[I]benzothieno[2,3-d]pyrimidin-7-yl][(1R,4R)-2-oxa-5-azabicyclo[2.2.1]hept-5-yl]methanone

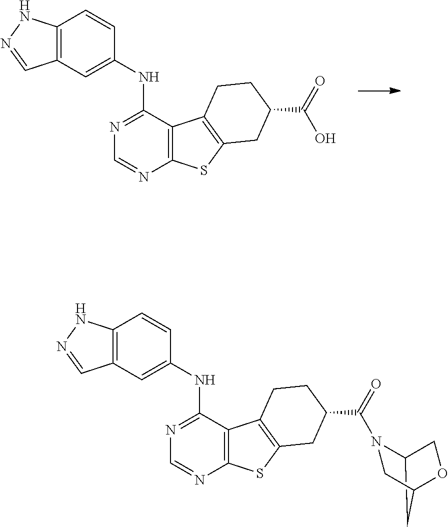 [SMILES: O=C(O)[C@H]1CCc2c(sc3ncnc(Nc4ccc5[nH]ncc5c4)c23)C1 ; O=C([C@H]1CCc2c(sc3ncnc(Nc4ccc5[nH]ncc5c4)c23)C1)N1CC2CC1CO2]

91 mg (249 μmol) (7S)-4-(1H-indazol-5-ylamino)-5,6,7,8-tetrahydro[1]benzothieno[2,3-d]pyrimidine-7-carboxylic acid (prepared according to intermediate example 1a) were transformed in analogy to example 1 using (1R,4R)-2-oxa-5-azabicyclo[2.2.1]heptane to give after working up and purification 58.2 mg (50%) of the title compound.

¹H-NMR (DMSO-d6): δ=1.74-1.90 (3H), 2.08 (1H), 2.74-3.36 (6H), 3.42-3.78 (3H), 4.60+4.66 (1H), 4.78+4.80 (1H), 7.46-7.54 (2H), 7.98 (1H), 8.05 (1H), 8.19 (1H), 8.30 (1H), 13.01 (1H) ppm.

### Example 12

### [(7S)-4-(1H-Indazol-5-ylamino)-5,6,7,8-tetrahydro[1]benzothieno[2,3-d]pyrimidin-7-yl](2-oxa-6-azaspiro[3.3]hept-6-yl)methanone

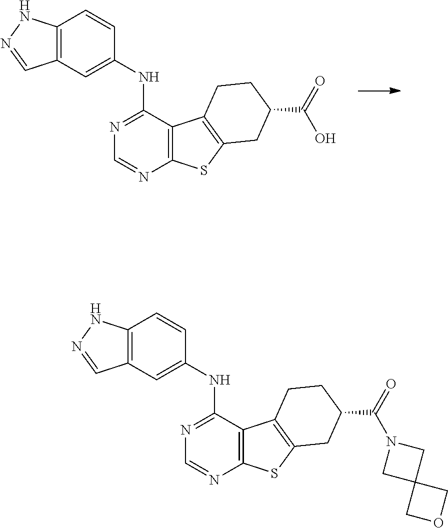 [SMILES: O=C(O)[C@H]1CCc2c(sc3ncnc(Nc4ccc5[nH]ncc5c4)c23)C1 ; O=C([C@H]1CCc2c(sc3ncnc(Nc4ccc5[nH]ncc5c4)c23)C1)N1CC2(COC2)C1]

91 mg (249 μmol) (7S)-4-(1H-indazol-5-ylamino)-5,6,7,8-tetrahydro[1]benzothieno[2,3-d]pyrimidine-7-carboxylic acid (prepared according to intermediate example 1a) were transformed in analogy to example 1 using 2-oxa-6-azaspiro[3.3]heptane to give after working up and purification 26.5 mg (23%) of the title compound.

¹H-NMR (DMSO-d6): δ=1.74 (1H), 2.04 (1H), 2.70 (1H), 2.88 (2H), 3.08-3.28 (2H), 4.05 (2H), 4.41 (2H), 4.67 (4H), 7.45-7.54 (2H), 7.97 (1H), 8.04 (1H), 8.19 (1H), 8.30 (1H), 13.03 (1H) ppm.

### Example 13

### (7S)-4-(1H-Indazol-5-ylamino)-N-(2-methoxyethyl)-N-methyl-5,6,7,8-tetrahydro[1]benzothieno[2,3-d]pyrimidine-7-carboxamide

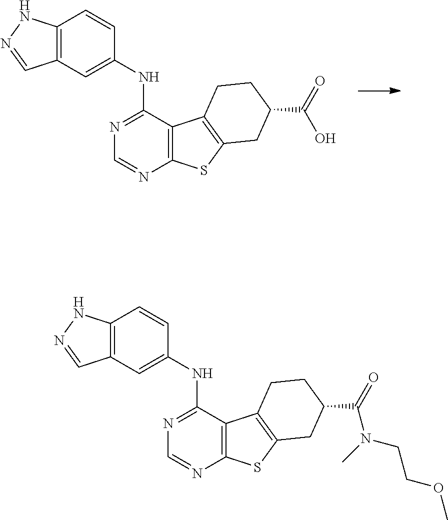 [SMILES: O=C(O)[C@H]1CCc2c(sc3ncnc(Nc4ccc5[nH]ncc5c4)c23)C1 ; COCCN(C)C(=O)[C@H]1CCc2c(sc3ncnc(Nc4ccc5[nH]ncc5c4)c23)C1]

91 mg (249 μmol) (7S)-4-(1H-indazol-5-ylamino)-5,6,7,8-tetrahydro[1]benzothieno[2,3-d]pyrimidine-7-carboxylic acid (prepared according to intermediate example 1a) were transformed in analogy to example 1 using 2-methoxy-N-methylethanamine to give after working up and purification 79.5 mg (70%) of the title compound.

¹H-NMR (DMSO-d6): δ=1.78 (1H), 2.05 (1H), 2.84-2.99 (2H), 2.87+3.12 (3H), 3.11-3.29 (3H), 3.25+3.27 (3H), 3.41-3.53 (3H), 3.59 (1H), 7.46-7.53 (2H), 7.98 (1H), 8.04 (1H), 8.18 (1H), 8.30 (1H), 13.01 (1H) ppm.

### Example 14

[(7S)-4-(1H-Indazol-5-ylamino)-5,6,7,8-tetrahydro[I]benzothieno[2,3-d]pyrimidin-7-yl](4-methylpiperazin-1-yl)methanone

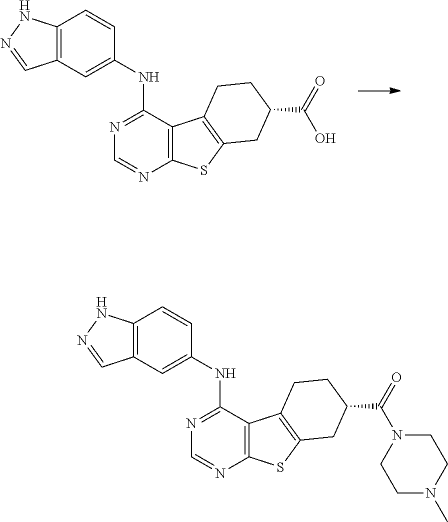 [SMILES: O=C(O)[C@H]1CCc2c(sc3ncnc(Nc4ccc5[nH]ncc5c4)c23)C1 ; CN1CCN(C(=O)[C@H]2CCc3c(sc4ncnc(Nc5ccc6[nH]ncc6c5)c34)C2)CC1]

91 mg (249 μmol) (7S)-4-(1H-indazol-5-ylamino)-5,6,7,8-tetrahydro[1]benzothieno[2,3-d]pyrimidine-7-carboxylic acid (prepared according to intermediate example 1a) were transformed in analogy to example 1 using 1-methylpiperazine to give after working up and purification 67.6 mg (58%) of the title compound.

¹H-NMR (DMSO-d6): δ=1.82 (1H), 2.05 (1H), 2.21 (3H), 2.28 (2H), 2.35 (2H), 2.89 (1H), 2.99 (1H), 3.14-3.30 (3H), 3.51 (2H), 3.57 (2H), 7.49 (1H), 7.53 (1H), 8.00 (1H), 8.06 (1H), 8.20 (1H), 8.32 (1H), 13.04 (1H) ppm.

### Example 15

### Reference Example

### [(7R)-4-(1H-Indazol-5-ylamino)-5,6,7,8-tetrahydro[1]benzothieno[2,3-d]pyrimidin-7-yl](4-methylpiperazin-1-yl)methanone

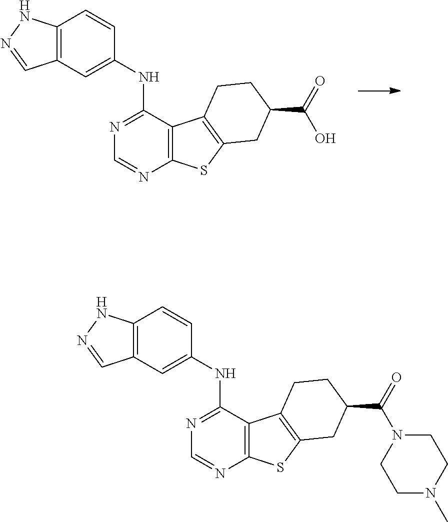 [SMILES: O=C(O)[C@@H]1CCc2c(sc3ncnc(Nc4ccc5[nH]ncc5c4)c23)C1 ; CN1CCN(C(=O)[C@@H]2CCc3c(sc4ncnc(Nc5ccc6[nH]ncc6c5)c34)C2)CC1]

100 mg (274 μmol) (7R)-4-(1H-indazol-5-ylamino)-5,6,7,8-tetrahydro[1]benzothieno[2,3-d]pyrimidine-7-carboxylic acid (prepared according to intermediate example 2a) were transformed in analogy to example 1 using 1-methylpiperazine to give after working up and purification 86.0 mg (67%) of the title compound.

¹H-NMR (DMSO-d6): δ=1.82 (1H), 2.05 (1H), 2.21 (3H), 2.28 (2H), 2.35 (2H), 2.89 (1H), 2.99 (1H), 3.14-3.30 (3H), 3.51 (2H), 3.57 (2H), 7.49 (1H), 7.53 (1H), 8.00 (1H), 8.06 (1H), 8.20 (1H), 8.32 (1H), 13.04 (1H) ppm.

### Example 16

### (7S)-4-(1H-Indazol-5-ylamino)-N,N-bis(2-methoxyethyl)-5,6,7,8-tetrahydro[1]benzothieno[2,3-d]pyrimidine-7-carboxamide

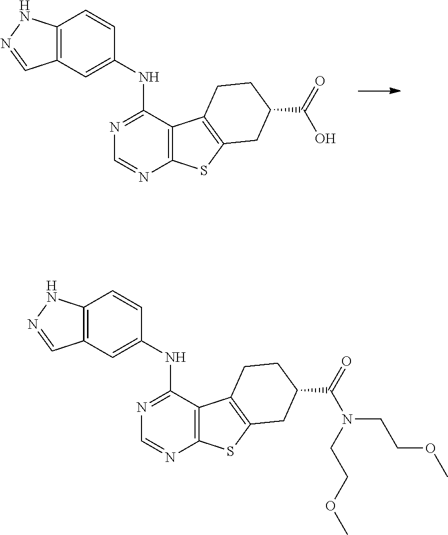 [SMILES: O=C(O)[C@H]1CCc2c(sc3ncnc(Nc4ccc5[nH]ncc5c4)c23)C1 ; COCCN(CCOC)C(=O)[C@H]1CCc2c(sc3ncnc(Nc4ccc5[nH]ncc5c4)c23)C1]

91 mg (249 μmol) (7S)-4-(1H-indazol-5-ylamino)-5,6,7,8-tetrahydro[1]benzothieno[2,3-d]pyrimidine-7-carboxylic acid (prepared according to intermediate example 1a) were transformed in analogy to example 1 using 2-methoxy-N-(2-methoxyethyl)ethanannine to give after working up and purification 73.1 mg (58%) of the title compound.

¹H-NMR (DMSO-d6): δ=1.79 (1H), 2.03 (1H), 2.84-2.99 (2H), 3.13-3.28 (3H), 3.25 (3H), 3.27 (3H), 3.36-3.55 (6H), 3.61 (2H), 7.46-7.54 (2H), 7.98 (1H), 8.04 (1H), 8.18 (1H), 8.30 (1H), 13.01 (1H) ppm.

### Example 17

### (3-Hydroxy-3-methylazetidin-1-yl)[(7S)-4-(1H-indazol-5-ylamino)-5,6,7,8-tetrahydro[1]benzothieno[2,3-d]pyrimidin-7-yl]methanone

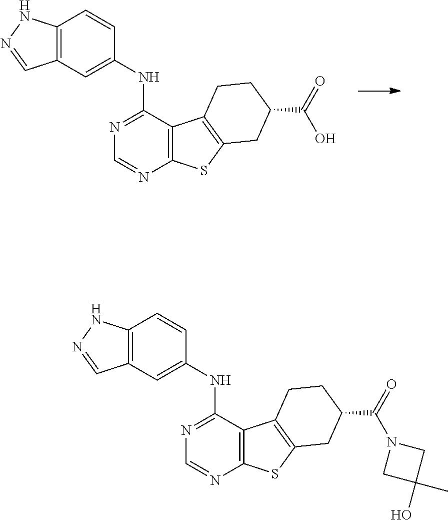 [SMILES: O=C(O)[C@H]1CCc2c(sc3ncnc(Nc4ccc5[nH]ncc5c4)c23)C1 ; CC1(O)CN(C(=O)[C@H]2CCc3c(sc4ncnc(Nc5ccc6[nH]ncc6c5)c34)C2)C1]

91 mg (249 μmol) (7S)-4-(1H-indazol-5-ylamino)-5,6,7,8-tetrahydro[1]benzothieno[2,3-d]pyrimidine-7-carboxylic acid (prepared according to intermediate example 1a) were transformed in analogy to example 1 using 3-methylazetidin-3-ol to give after working up and purification 84.0 mg (78%) of the title compound.

¹H-NMR (DMSO-d6): δ=1.39 (3H), 1.76 (1H), 2.05 (1H), 2.76 (1H), 2.90 (2H), 3.17 (1H), 3.27 (1H), 3.67-3.78 (2H), 3.98-4.14 (2H), 5.66 (1H), 7.45-7.54 (2H), 7.98 (1H), 8.04 (1H), 8.18 (1H), 8.30 (1H), 13.00 (1H) ppm.

### Example 18

### (7S)—N-(2-Hydroxy-2-methylpropyl)-4-(1H-indazol-5-ylamino)-N-methyl-5,6,7,8-tetrahydro[1]benzothieno[2,3-d]pyrimidine-7-carboxamide

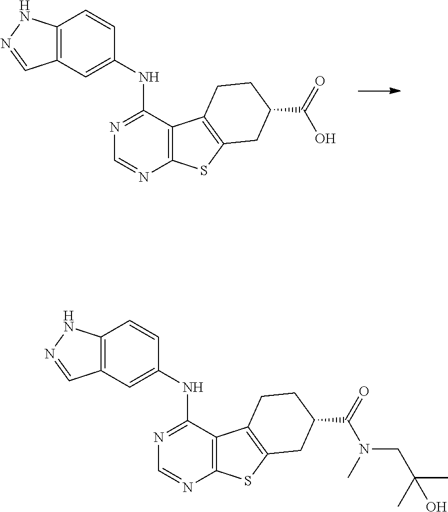 [SMILES: O=C(O)[C@H]1CCc2c(sc3ncnc(Nc4ccc5[nH]ncc5c4)c23)C1 ; CN(CC(C)(C)O)C(=O)[C@H]1CCc2c(sc3ncnc(Nc4ccc5[nH]ncc5c4)c23)C1]

91 mg (249 μmol) (7S)-4-(1H-indazol-5-ylamino)-5,6,7,8-tetrahydro[1]benzothieno[2,3-d]pyrimidine-7-carboxylic acid (prepared according to intermediate example 1a) were transformed in analogy to example 1 using 2-methyl-1-(methylamino)propan-2-ol to give after working up and purification 53.1 mg (45%) of the title compound.

¹H-NMR (DMSO-d6): δ=1.03-1.16 (6H), 1.78 (1H), 2.02+2.10 (1H), 2.82-2.99 (2H), 2.95+3.20 (3H), 3.13-3.42 (5H), 4.50+4.58 (1H), 7.46-7.54 (2H), 7.98 (1H), 8.04 (1H), 8.13-8.23 (1H), 8.30 (1H), 13.01 (1H) ppm.

### Example 19

### (7S)—N-Ethyl-N-(2-hydroxyethyl)-4-(1H-indazol-5-ylamino)-5, 6, 7,8-tetrahydro[1]benzothieno[2,3-d]pyrimidine-7-carboxamide

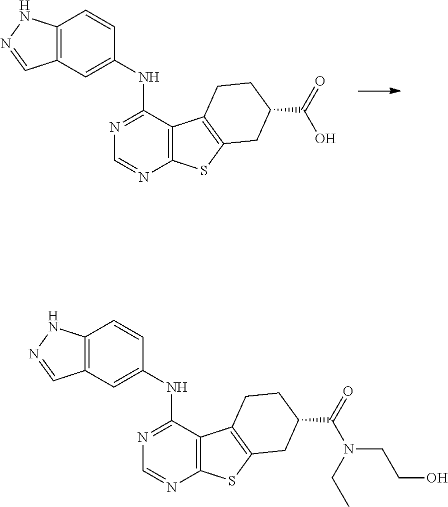 [SMILES: O=C(O)[C@H]1CCc2c(sc3ncnc(Nc4ccc5[nH]ncc5c4)c23)C1 ; CCN(CCO)C(=O)[C@H]1CCc2c(sc3ncnc(Nc4ccc5[nH]ncc5c4)c23)C1]

91 mg (249 μmol) (7S)-4-(1H-indazol-5-ylamino)-5,6,7,8-tetrahydro[1]benzothieno[2,3-d]pyrimidine-7-carboxylic acid (prepared according to intermediate example 1a) were transformed in analogy to example 1 using 2-(ethylamino)ethanol to give after working up and purification 45.8 mg (40%) of the title compound.

¹H-NMR (DMSO-d6): δ=0.98-1.10 (3H), 1.80 (1H), 2.03 (1H), 2.81-3.58 (11H), 4.68+4.86 (1H), 7.45-7.54 (2H), 7.98 (1H), 8.05 (1H), 8.19 (1H), 8.30 (1H), 13.02 (1H) ppm.

### Example 20

### (7S)-4-[(6-Methoxy-1H-indazol-5-yl)amino]-N,N-dimethyl-5,6,7,8-tetrahydro[1]benzothieno[2,3-d]pyrimidine-7-carboxamide

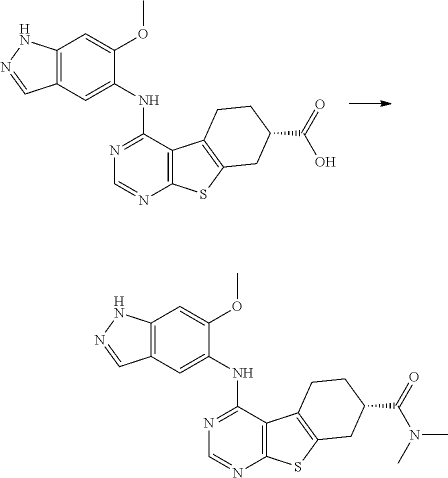 [SMILES: COc1cc2[nH]ncc2cc1Nc1ncnc2sc3c(c12)CC[C@H](C(=O)O)C3 ; COc1cc2[nH]ncc2cc1Nc1ncnc2sc3c(c12)CC[C@H](C(=O)N(C)C)C3]

250 mg (632 μmol) (7S)-4-[(6-methoxy-1H-indazol-5-yl)amino]-5,6,7,8-tetrahydro[1]benzothieno[2,3-d]pyrimidine-7-carboxylic acid (prepared according to intermediate example 20a) were transformed in analogy to example 1 to give after working up and purification 212 mg (75%) of the title compound.

¹H-NMR (DMSO-d6): δ=1.83 (1H), 2.14 (1H), 2.87 (3H), 2.89-2.98 (2H), 3.10 (3H), 3.14-3.28 (3H), 3.98 (3H), 7.09 (1H), 7.99 (1H), 8.21 (1H), 8.45 (1H), 8.77 (1H), 12.84 (1H) ppm.

### Example 20a

### (7S)-4-[(6-Methoxy-1H-indazol-5-yl)amino]-5,6,7,8-tetrahydro[1]benzothieno[2,3-d]pyrimidine-7-carboxylic acid

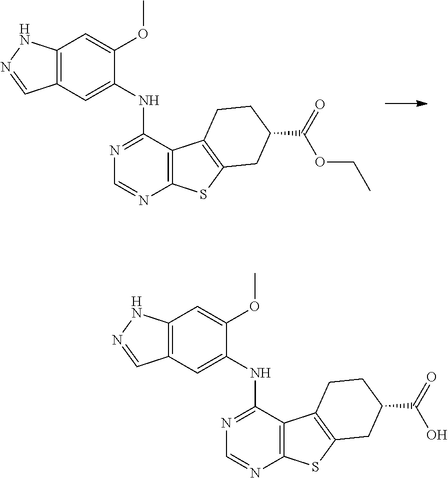 [SMILES: CCOC(=O)[C@H]1CCc2c(sc3ncnc(Nc4cc5cn[nH]c5cc4OC)c23)C1 ; COc1cc2[nH]ncc2cc1Nc1ncnc2sc3c(c12)CC[C@H](C(=O)O)C3]

4.64 g (10.96 mmol) ethyl (7S)-4-[(6-methoxy-1H-indazol-5-yl)amino]-5,6,7,8-tetrahydro[1]benzothieno[2,3-d]pyrimidine-7-carboxylate (prepared according to intermediate example 20b) were transformed in analogy to intermediate example 1a to give after working up and purification 4.33 g (95%) of the title compound.

### Example 20b

### Ethyl (7S)-4-[(6-methoxy-1H-indazol-5-yl)amino]-5,6,7,8-tetrahydro[1]benzothieno[2,3-d]pyrimidine-7-carboxylate

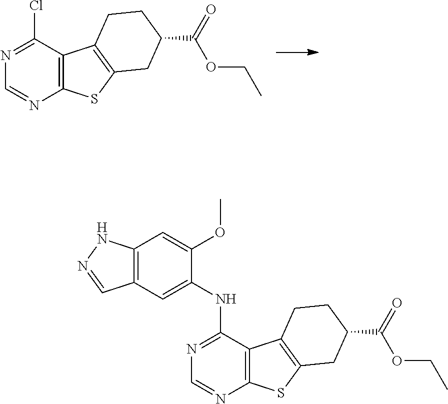 [SMILES: CCOC(=O)[C@H]1CCc2c(sc3ncnc(Cl)c23)C1 ; CCOC(=O)[C@H]1CCc2c(sc3ncnc(Nc4cc5cn[nH]c5cc4OC)c23)C1]

4.65 g (15.68 mmol) ethyl (7S)-4-chloro-5,6,7,8-tetrahydro[1]benzothieno[2,3-d]pyrimidine-7-carboxylate (prepared according to intermediate example 1c) were transformed in analogy to intermediate example 1b using 6-methoxy-1H-indazol-5-amine (CAS-No: 749223-61-8) to give after working up and purification 4.64 g (63%) of the title compound.

### Example 21

### Azetidin-1-yl{(7S)-4-[(6-methoxy-1H-indazol-5-yl)amino]-5,6,7,8-tetrahydro[1]benzothieno[2,3-d]pyrimidin-7-yl}methanone

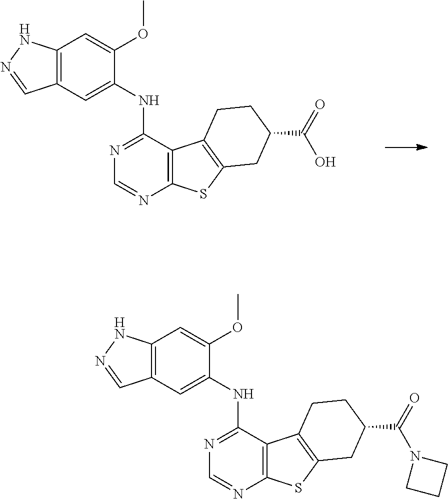 [SMILES: COc1cc2[nH]ncc2cc1Nc1ncnc2sc3c(c12)CC[C@H](C(=O)O)C3 ; COc1cc2[nH]ncc2cc1Nc1ncnc2sc3c(c12)CC[C@H](C(=O)N1CCC1)C3]

150 mg (379 μmol) (7S)-4-[(6-methoxy-1H-indazol-5-yl)amino]-5,6,7,8-tetrahydro[1]benzothieno[2,3-d]pyrimidine-7-carboxylic acid (prepared according to intermediate example 20a) were transformed in analogy to example 1 using azetidine to give after working up and purification 102 mg (59%) of the title compound.

¹H-NMR (DMSO-d6): δ=1.80 (1H), 2.13 (1H), 2.22 (2H), 2.75 (1H), 2.89 (2H), 3.07-3.20 (1H), 3.25 (1H), 3.88 (2H), 3.98 (3H), 4.25 (2H), 7.08 (1H), 7.99 (1H), 8.21 (1H), 8.45 (1H), 8.77 (1H), 12.83 (1H) ppm.

### Example 22

### Reference Example

### Azetidin-1-yl{(7R)-4-[(6-methoxy-1H-indazol-5-yl)amino]-5,6,7,8-tetrahydro[1]benzothieno[2,3-d]pyrimidin-7-yl}methanone

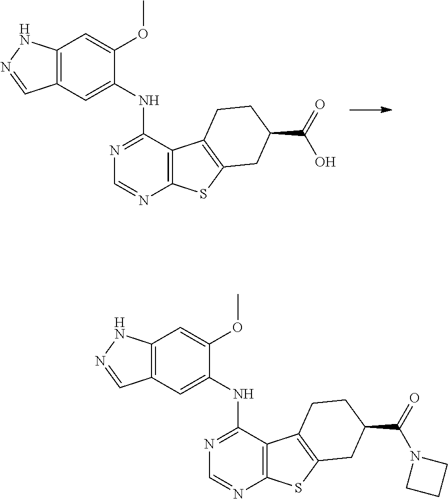 [SMILES: COc1cc2[nH]ncc2cc1Nc1ncnc2sc3c(c12)CC[C@@H](C(=O)O)C3 ; COc1cc2[nH]ncc2cc1Nc1ncnc2sc3c(c12)CC[C@@H](C(=O)N1CCC1)C3]

150 mg (379 μmol) (7R)-4-[(6-methoxy-1H-indazol-5-yl)amino]-5,6,7,8-tetrahydro[1]benzothieno[2,3-d]pyrimidine-7-carboxylic acid (prepared according to intermediate example 22a) were transformed in analogy to example 1 using azetidine to give after working up and purification 63.3 mg (35%) of the title compound.

¹H-NMR (DMSO-d6): δ=1.80 (1H), 2.13 (1H), 2.22 (2H), 2.75 (1H), 2.89 (2H), 3.07-3.20 (1H), 3.25 (1H), 3.88 (2H), 3.98 (3H), 4.25 (2H), 7.08 (1H), 7.99 (1H), 8.21 (1H), 8.45 (1H), 8.77 (1H), 12.83 (1H) ppm.

### Example 22a

### (7R)-4-[(6-Methoxy-1H-indazol-5-yl)amino]-5,6,7,8-tetrahydro[1]benzothieno[2,3-d]pyrimidine-7-carboxylic acid

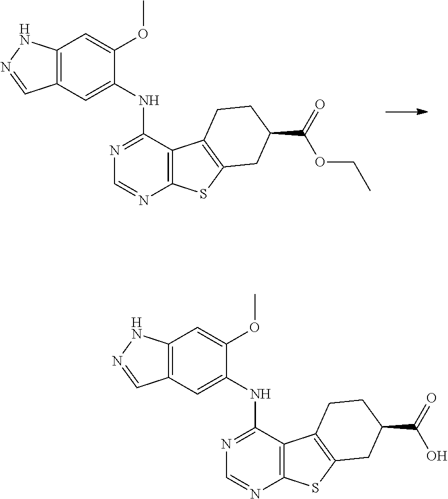 [SMILES: CCOC(=O)[C@@H]1CCc2c(sc3ncnc(Nc4cc5cn[nH]c5cc4OC)c23)C1 ; COc1cc2[nH]ncc2cc1Nc1ncnc2sc3c(c12)CC[C@@H](C(=O)O)C3]

4.03 g (9.52 mmol) ethyl (7R)-4-[(6-methoxy-1H-indazol-5-yl)amino]-5,6,7,8-tetrahydro[1]benzothieno[2,3-d]pyrimidine-7-carboxylate (prepared according to intermediate example 22b) were transformed in analogy to intermediate example 1a to give after working up and purification 3.13 g (79%) of the title compound.

### Example 22b

### Ethyl (7R)-4-[(6-methoxy-1H-indazol-5-yl)amino]-5,6,7,8-tetrahydro[1]benzothieno[2,3-d]pyrimidine-7-carboxylate

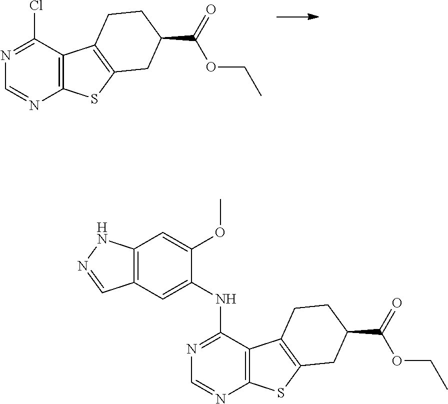 [SMILES: CCOC(=O)[C@@H]1CCc2c(sc3ncnc(Cl)c23)C1 ; CCOC(=O)[C@@H]1CCc2c(sc3ncnc(Nc4cc5cn[nH]c5cc4OC)c23)C1]

4.00 g (13.48 mmol) ethyl (7R)-4-chloro-5,6,7,8-tetrahydro[1]benzothieno[2,3-d]pyrimidine-7-carboxylate (prepared according to intermediate example 2c) were transformed in analogy to intermediate example 1b using 6-methoxy-1H-indazol-5-amine to give after working up and purification 4.03 g (67%) of the title compound.

### Example 23

### {(7S)-4-[(6-Methoxy-1H-indazol-5-yl)amino]-5,6,7,8-tetrahydro[1]benzothieno[2,3-d]pyrimidin-7-yl}(pyrrolidin-1-yl)methanone

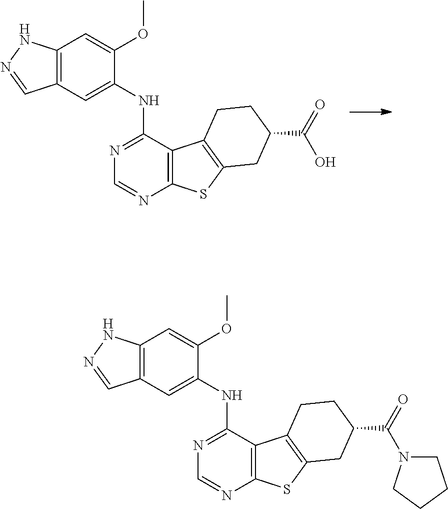 [SMILES: COc1cc2[nH]ncc2cc1Nc1ncnc2sc3c(c12)CC[C@H](C(=O)O)C3 ; COc1cc2[nH]ncc2cc1Nc1ncnc2sc3c(c12)CC[C@H](C(=O)N1CCCC1)C3]

100 mg (253 μmol) (7S)-4-[(6-methoxy-1H-indazol-5-yl)amino]-5,6,7,8-tetrahydro[1]benzothieno[2,3-d]pyrimidine-7-carboxylic acid (prepared according to intermediate example 20a) were transformed in analogy to example 1 using pyrrolidine to give after working up and purification 57.3 mg (48%) of the title compound.

¹H-NMR (DMSO-d6): δ=1.76-1.94 (5H), 2.19 (1H), 2.90-3.01 (3H), 3.10-3.39 (4H), 3.57 (2H), 3.98 (3H), 7.08 (1H), 7.99 (1H), 8.22 (1H), 8.46 (1H), 8.78 (1H), 12.84 (1H) ppm.

### Example 24

### (7S)—N-Ethyl-4-[(6-methoxy-1H-indazol-5-yl)amino]-N-methyl-5,6,7,8-tetrahydro[1]benzothieno[2,3-d]pyrimidine-7-carboxamide

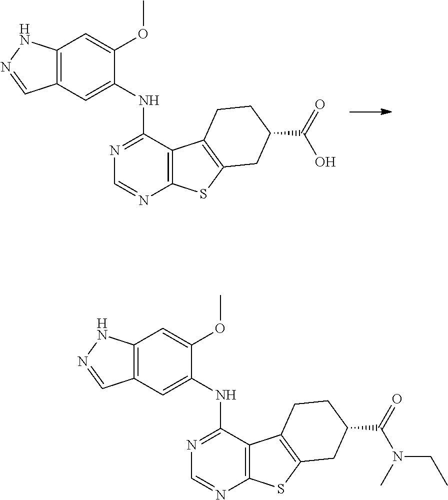 [SMILES: COc1cc2[nH]ncc2cc1Nc1ncnc2sc3c(c12)CC[C@H](C(=O)O)C3 ; CCN(C)C(=O)[C@H]1CCc2c(sc3ncnc(Nc4cc5cn[nH]c5cc4OC)c23)C1]

150 mg (379 μmol) (7S)-4-[(6-methoxy-1H-indazol-5-yl)amino]-5,6,7,8-tetrahydro[1]benzothieno[2,3-d]pyrimidine-7-carboxylic acid (prepared according to intermediate example 20a) were transformed in analogy to example 1 using N-methylethanamine to give after working up and purification 136 mg (78%) of the title compound.

¹H-NMR (DMSO-d6): δ=1.03+1.15 (3H), 1.84 (1H), 2.12 (1H), 2.85+3.07 (3H), 2.86-3.01 (2H), 3.09-3.54 (5H), 3.98 (3H), 7.08 (1H), 7.99 (1H), 8.21 (1H), 8.46 (1H), 8.78 (1H), 12.84 (1H) ppm.

### Example 25

### Reference Example

### (7R)—N-Ethyl-4-[(6-methoxy-1H-indazol-5-yl)amino]-N-methyl-5,6,7,8-tetrahydro[1]benzothieno[2,3-d]pyrimidine-7-carboxamide

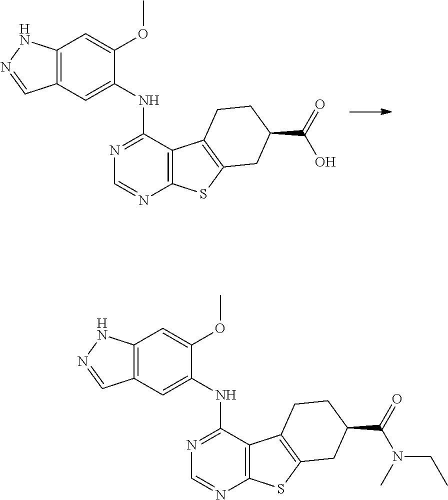 [SMILES: COc1cc2[nH]ncc2cc1Nc1ncnc2sc3c(c12)CC[C@@H](C(=O)O)C3 ; CCN(C)C(=O)[C@@H]1CCc2c(sc3ncnc(Nc4cc5cn[nH]c5cc4OC)c23)C1]

150 mg (379 μmol) (7R)-4-[(6-methoxy-1H-indazol-5-yl)amino]-5,6,7,8-tetrahydro[1]benzothieno[2,3-d]pyrimidine-7-carboxylic acid (prepared according to intermediate example 22a) were transformed in analogy to example 1 using N-methylethanamine to give after working up and purification 101 mg (58%) of the title compound.

¹H-NMR (DMSO-d6): δ=1.03+1.15 (3H), 1.84 (1H), 2.12 (1H), 2.85+3.07 (3H), 2.86-3.01 (2H), 3.09-3.54 (5H), 3.98 (3H), 7.08 (1H), 7.99 (1H), 8.21 (1H), 8.46 (1H), 8.78 (1H), 12.84 (1H) ppm.

### Example 26

### (7S)-4-[(6-Methoxy-1H-indazol-5-yl)amino]-N-methyl-N-(propan-2-yl)-5, 6, 7,8-tetrahydro[1]benzothieno[2,3-d]pyrimidine-7-carboxamide

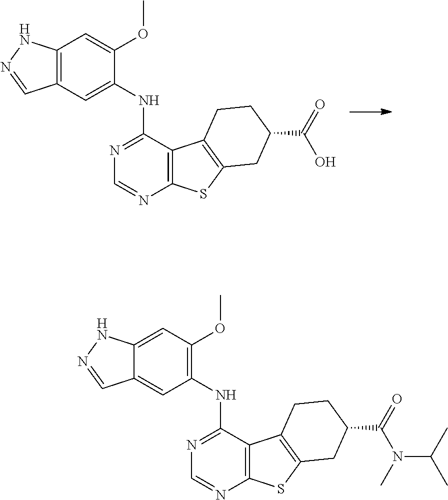 [SMILES: COc1cc2[nH]ncc2cc1Nc1ncnc2sc3c(c12)CC[C@H](C(=O)O)C3 ; COc1cc2[nH]ncc2cc1Nc1ncnc2sc3c(c12)CC[C@H](C(=O)N(C)C(C)C)C3]

150 mg (379 μmol) (7S)-4-[(6-methoxy-1H-indazol-5-yl)amino]-5,6,7,8-tetrahydro[1]benzothieno[2,3-d]pyrimidine-7-carboxylic acid (prepared according to intermediate example 20a) were transformed in analogy to example 1 using N-methylpropan-2-amine to give after working up and purification 104 mg (58%) of the title compound.

¹H-NMR (DMSO-d6): δ=1.05+1.18 (6H), 1.84 (1H), 2.11 (1H), 2.71+2.91 (3H), 2.83-3.28 (5H), 3.98 (3H), 4.29+4.72 (1H), 7.08 (1H), 7.99 (1H), 8.22 (1H), 8.46 (1H), 8.78 (1H), 12.85 (1H) ppm.

### Example 27

### Reference Example

### (7R)-4-[(6-Methoxy-1H-indazol-5-yl)amino]-N-methyl-N-(propan-2-yl)-5, 6, 7,8-tetrahydro[1]benzothieno[2,3-d]pyrimidine-7-carboxamide

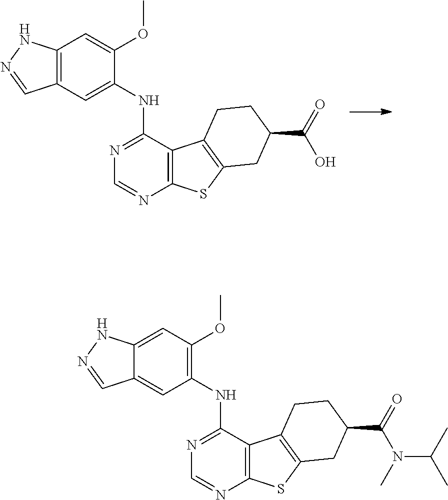 [SMILES: COc1cc2[nH]ncc2cc1Nc1ncnc2sc3c(c12)CC[C@@H](C(=O)O)C3 ; COc1cc2[nH]ncc2cc1Nc1ncnc2sc3c(c12)CC[C@@H](C(=O)N(C)C(C)C)C3]

150 mg (379 μmol) (7R)-4-[(6-methoxy-1H-indazol-5-yl)amino]-5,6,7,8-tetrahydro[1]benzothieno[2,3-d]pyrimidine-7-carboxylic acid (prepared according to intermediate example 22a) were transformed in analogy to example 1 using N-methylpropan-2-amine to give after working up and purification 91.7 mg (51%) of the title compound.

¹H-NMR (DMSO-d6): δ=1.05+1.18 (6H), 1.84 (1H), 2.11 (1H), 2.71+2.91 (3H), 2.83-3.28 (5H), 3.98 (3H), 4.29+4.72 (1H), 7.08 (1H), 7.99 (1H), 8.22 (1H), 8.46 (1H), 8.78 (1H), 12.85 (1H) ppm.

### Example 28

### {(7S)-4-[(6-Methoxy-1H-indazol-5-yl)amino]-5,6,7,8-tetrahydro[1]benzothieno[2,3-d]pyrimidin-7-yl}(morpholin-4-yl)methanone

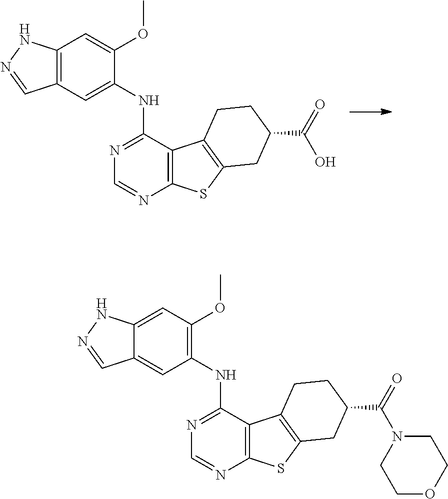 [SMILES: COc1cc2[nH]ncc2cc1Nc1ncnc2sc3c(c12)CC[C@H](C(=O)O)C3 ; COc1cc2[nH]ncc2cc1Nc1ncnc2sc3c(c12)CC[C@H](C(=O)N1CCOCC1)C3]

150 mg (379 μmol) (7S)-4-[(6-methoxy-1H-indazol-5-yl)amino]-5,6,7,8-tetrahydro[1]benzothieno[2,3-d]pyrimidine-7-carboxylic acid (prepared according to intermediate example 20a) were transformed in analogy to example 1 using morpholine to give after working up and purification 91.1 mg (49%) of the title compound.

¹H-NMR (DMSO-d6): δ=1.85 (1H), 2.14 (1H), 2.85-3.04 (2H), 3.11-3.39 (3H), 3.44-3.68 (8H), 3.98 (3H), 7.08 (1H), 7.99 (1H), 8.21 (1H), 8.46 (1H), 8.76 (1H), 12.86 (1H) ppm.

### Example 29

### Reference Example

### {(7R)-4-[(6-Methoxy-1H-indazol-5-yl)amino]-5,6,7,8-tetrahydro[1]benzothieno[2,3-d]pyrimidin-7-yl}(morpholin-4-yl)methanone

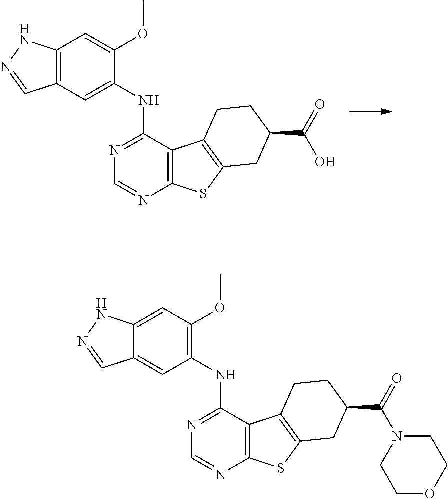 [SMILES: COc1cc2[nH]ncc2cc1Nc1ncnc2sc3c(c12)CC[C@@H](C(=O)O)C3 ; COc1cc2[nH]ncc2cc1Nc1ncnc2sc3c(c12)CC[C@@H](C(=O)N1CCOCC1)C3]

150 mg (379 μmol) (7R)-4-[(6-methoxy-1H-indazol-5-yl)amino]-5,6,7,8-tetrahydro[1]benzothieno[2,3-d]pyrimidine-7-carboxylic acid (prepared according to intermediate example 22a) were transformed in analogy to example 1 using morpholine to give after working up and purification 80.0 mg (43%) of the title compound.

¹H-NMR (DMSO-d6): δ=1.85 (1H), 2.14 (1H), 2.85-3.04 (2H), 3.11-3.39 (3H), 3.44-3.68 (8H), 3.98 (3H), 7.08 (1H), 7.99 (1H), 8.21 (1H), 8.46 (1H), 8.76 (1H), 12.86 (1H) ppm.

### Example 30

### {(7S)-4-[(6-Methoxy-1H-indazol-5-yl)amino]-5,6,7,8-tetrahydro[1]benzothieno[2,3-d]pyrimidin-7-yl}[(3S)-3-methylmorpholin-4-yl]methanone

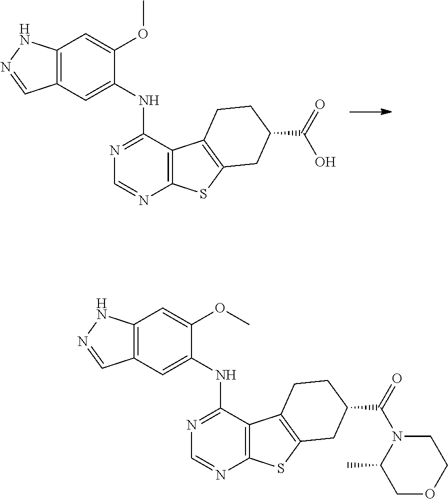 [SMILES: COc1cc2[nH]ncc2cc1Nc1ncnc2sc3c(c12)CC[C@H](C(=O)O)C3 ; COc1cc2[nH]ncc2cc1Nc1ncnc2sc3c(c12)CC[C@H](C(=O)N1CCOC[C@@H]1C)C3]

100 mg (253 μmol) (7S)-4-[(6-methoxy-1H-indazol-5-yl)amino]-5,6,7,8-tetrahydro[1]benzothieno[2,3-d]pyrimidine-7-carboxylic acid (prepared according to intermediate example 20a) were transformed in analogy to example 1 using (3S)-3-methylmorpholine to give after working up and purification 70.4 mg (55%) of the title compound.

¹H-NMR (DMSO-d6): δ=1.16+1.29 (3H), 1.82 (1H), 2.12 (1H), 2.83-4.49 (12H), 3.98 (3H), 7.09 (1H), 8.00 (1H), 8.22 (1H), 8.46 (1H), 8.78 (1H), 12.85 (1H) ppm.

### Example 31

### {(7S)-4-[(6-Methoxy-1H-indazol-5-yl)amino]-5,6,7,8-tetrahydro[1]benzothieno[2,3-d]pyrimidin-7-yl}[(3R)-3-methylmorpholin-4-yl]methanone

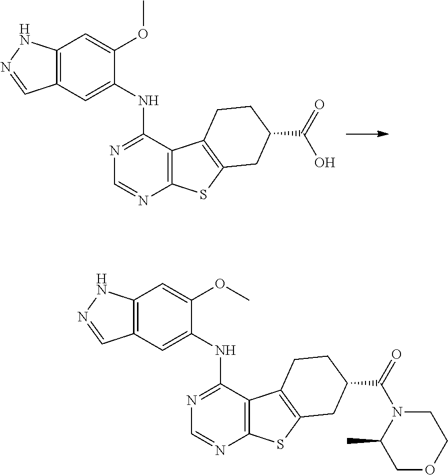 [SMILES: COc1cc2[nH]ncc2cc1Nc1ncnc2sc3c(c12)CC[C@H](C(=O)O)C3 ; COc1cc2[nH]ncc2cc1Nc1ncnc2sc3c(c12)CC[C@H](C(=O)N1CCOC[C@H]1C)C3]

100 mg (253 μmol) (7S)-4-[(6-methoxy-1H-indazol-5-yl)amino]-5,6,7,8-tetrahydro[1]benzothieno[2,3-d]pyrimidine-7-carboxylic acid (prepared according to intermediate example 20a) were transformed in analogy to example 1 using (3R)-3-methylmorpholine to give after working up and purification 64.1 mg (50%) of the title compound.

¹H-NMR (DMSO-d6): δ=1.15+1.31 (3H), 1.91 (1H), 2.11 (1H), 2.83-4.46 (12H), 3.98 (3H), 7.09 (1H), 7.99 (1H), 8.21 (1H), 8.46 (1H), 8.76 (1H), 12.85 (1H) ppm.

### Example 32

### {(7S)-4-[(6-Methoxy-1H-indazol-5-yl)amino]-5,6,7,8-tetrahydro[1]benzothieno[2,3-d]pyrimidin-7-yl}(2-oxa-6-azaspiro[3.3]hept-6-yl)methanone

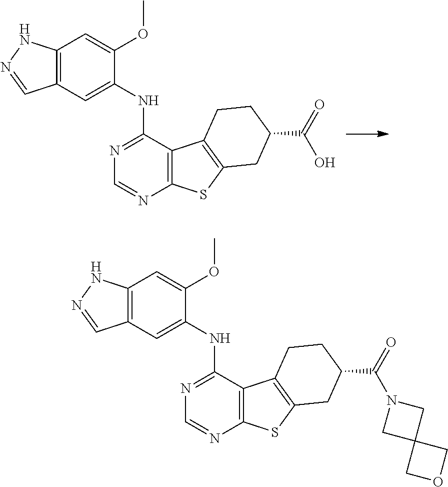

100 mg (253 μmol) (7S)-4-[(6-methoxy-1H-indazol-5-yl)amino]-5,6,7,8-tetrahydro[1]benzothieno[2,3-d]pyrimidine-7-carboxylic acid (prepared according to intermediate example 20a) were transformed in analogy to example 1 using 2-oxa-6-azaspiro[3.3]heptane to give after working up and purification 32.0 mg (25%) of the title compound.

¹H-NMR (DMSO-d6): δ=1.79 (1H), 2.13 (1H), 2.74 (1H), 2.81-2.94 (2H), 3.08-3.19 (1H), 3.25 (1H), 3.98 (3H), 4.06 (2H), 4.38-4.46 (2H), 4.65-4.72 (4H), 7.09 (1H), 7.99 (1H), 8.20 (1H), 8.45 (1H), 8.76 (1H), 12.84 (1H) ppm.

### Example 33

### {(7S)-4-[(6-Methoxy-1H-indazol-5-yl)amino]-5,6,7,8-tetrahydro[1]benzothieno[2,3-d]pyrimidin-7-yl}[(1S,4S)-2-oxa-5-azabicyclo[2.2.1]hept-5-yl]methanone

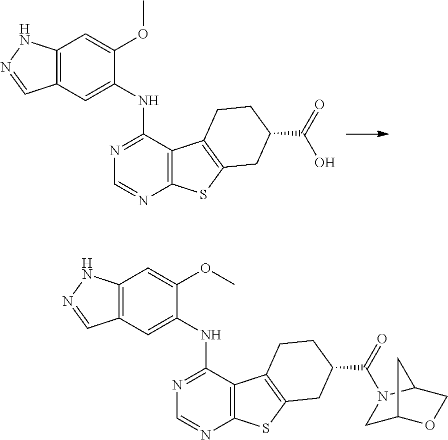 [SMILES: COc1cc2[nH]ncc2cc1Nc1ncnc2sc3c(c12)CC[C@H](C(=O)O)C3 ; COc1cc2[nH]ncc2cc1Nc1ncnc2sc3c(c12)CC[C@H](C(=O)N1CC2CC1CO2)C3]

45.0 mg (114 μmol) (7S)-4-[(6-methoxy-1H-indazol-5-yl)amino]-5,6,7,8-tetrahydro[1]benzothieno[2,3-d]pyrimidine-7-carboxylic acid (prepared according to intermediate example 20a) were transformed in analogy to example 1 using (1S,4S)-2-oxa-5-azabicyclo[2.2.1]heptane to give after working up and purification 35.8 mg (63%) of the title compound.

¹H-NMR (DMSO-d6): δ=1.76-1.93 (3H), 2.14 (1H), 2.80-3.34 (6H), 3.54+3.65 (1H), 3.59+3.72 (1H), 3.77 (1H), 3.97 (3H), 4.61+4.77 (1H), 4.66+4.87 (1H), 7.08 (1H), 7.99 (1H), 8.20 (1H), 8.45 (1H), 8.75+8.78 (1H), 12.83 (1H) ppm.

### Example 34

### {(7S)-4-[(6-Methoxy-1H-indazol-5-yl)amino]-5,6,7,8-tetrahydro[1]benzothieno[2,3-d]pyrimidin-7-yl}[(1R,4R)-2-oxa-5-azabicyclo[2.2.1]hept-5-yl]methanone

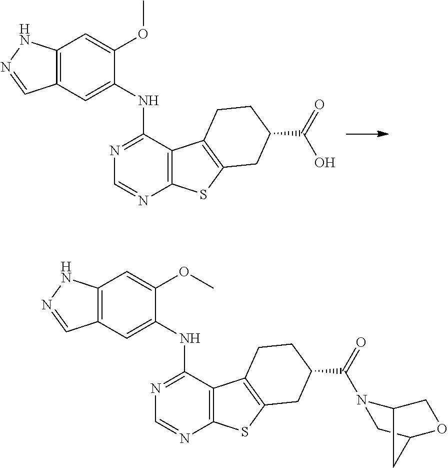 [SMILES: COc1cc2[nH]ncc2cc1Nc1ncnc2sc3c(c12)CC[C@H](C(=O)O)C3 ; COc1cc2[nH]ncc2cc1Nc1ncnc2sc3c(c12)CC[C@H](C(=O)N1CC2CC1CO2)C3]

60 mg (152 μmol) (7S)-4-[(6-methoxy-1H-indazol-5-yl)amino]-5,6,7,8-tetrahydro[1]benzothieno[2,3-d]pyrimidine-7-carboxylic acid (prepared according to intermediate example 20a) were transformed in analogy to example 1 using (1R,4R)-2-oxa-5-azabicyclo[2.2.1]heptane to give after working up and purification 44.5 mg (62%) of the title compound.

¹H-NMR (DMSO-d6): δ=1.74-1.93 (3H), 2.17 (1H), 2.75-3.29 (6H), 3.49-3.79 (3H), 3.99 (3H), 4.61+4.78 (1H), 4.67+4.87 (1H), 7.09 (1H), 7.99 (1H), 8.23 (1H), 8.46 (1H), 8.79+8.81 (1H), 12.85 (1H) ppm.

### Example 35

### 1-({(7S)-4-[(6-Methoxy-1H-indazol-5-yl)amino]-5,6,7,8-tetrahydro[1]benzothieno[2,3-d]pyrimidin-7-yl}carbonyl)azetidine-3-carbonitrile

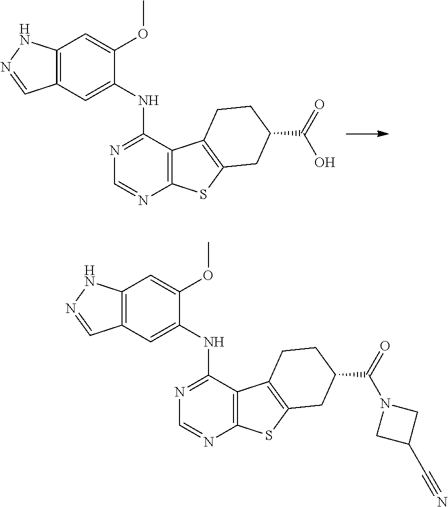 [SMILES: COc1cc2[nH]ncc2cc1Nc1ncnc2sc3c(c12)CC[C@H](C(=O)O)C3 ; COc1cc2[nH]ncc2cc1Nc1ncnc2sc3c(c12)CC[C@H](C(=O)N1CC(C#N)C1)C3]

100 mg (253 μmol) (7S)-4-[(6-methoxy-1H-indazol-5-yl)amino]-5,6,7,8-tetrahydro[1]benzothieno[2,3-d]pyrimidine-7-carboxylic acid (prepared according to intermediate example 20a) were transformed in analogy to example 1 using azetidine-3-carbonitrile to give after working up and purification 20.0 mg (16%) of the title compound.

¹H-NMR (DMSO-d6): δ=1.80 (1H), 2.16 (1H), 2.77 (1H), 2.82-3.01 (2H), 3.08-3.19 (1H), 3.25 (1H), 3.81 (1H), 3.97+3.99 (3H), 4.05 (1H), 4.19 (1H), 4.45-4.59 (2H), 7.09 (1H), 7.99 (1H), 8.21 (1H), 8.45 (1H), 8.76 (1H), 12.84 (1H) ppm.

### Example 36

### (3-hydroxy-3-Methylazetidin-1-yl){(7S)-4-[(6-methoxy-1H-indazol-5-yl)amino]-5,6,7,8-tetrahydro[1]benzothieno[2,3-d]pyrimidin-7-yl}methanone

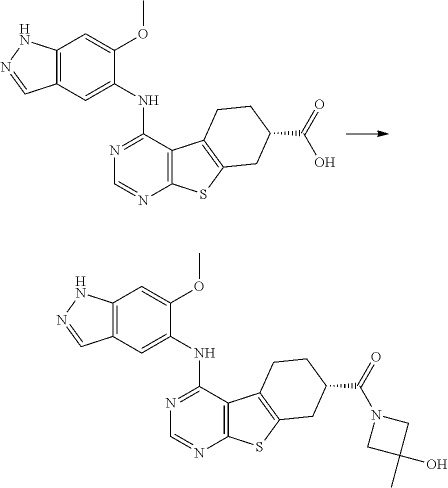 [SMILES: COc1cc2[nH]ncc2cc1Nc1ncnc2sc3c(c12)CC[C@H](C(=O)O)C3 ; COc1cc2[nH]ncc2cc1Nc1ncnc2sc3c(c12)CC[C@H](C(=O)N1CC(C)(O)C1)C3]

100 mg (253 μmol) (7S)-4-[(6-methoxy-1H-indazol-5-yl)amino]-5,6,7,8-tetrahydro[1]benzothieno[2,3-d]pyrimidine-7-carboxylic acid (prepared according to intermediate example 20a) were transformed in analogy to example 1 using 3-methylazetidin-3-ol to give after working up and purification 10.6 mg (9%) of the title compound.

¹H-NMR (DMSO-d6): δ=1.40 (3H), 1.82 (1H), 2.13 (1H), 2.78 (1H), 2.90 (2H), 3.15 (1H), 3.25 (1H), 3.68-3.77 (2H), 3.98 (3H), 4.02-4.13 (2H), 5.65 (1H), 7.09 (1H), 7.99 (1H), 8.20 (1H), 8.45 (1H), 8.77 (1H), 12.84 (1H) ppm.

### Example 37

### (7S)—N-(2-Hydroxyethyl)-4-[(6-methoxy-1H-indazol-5-yl)amino]-N-(oxetan-3-yl)-5,6,7,8-tetrahydro[1]benzothieno[2,3-d]pyrimidine-7-carboxamide

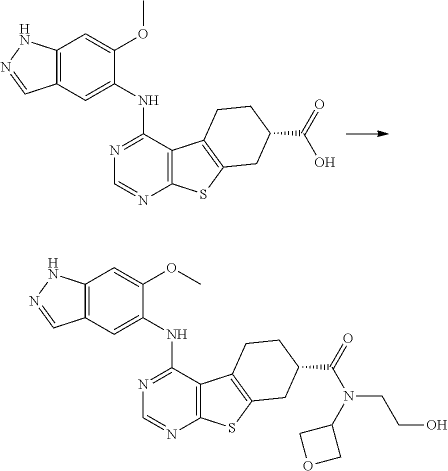 [SMILES: COc1cc2[nH]ncc2cc1Nc1ncnc2sc3c(c12)CC[C@H](C(=O)O)C3 ; COc1cc2[nH]ncc2cc1Nc1ncnc2sc3c(c12)CC[C@H](C(=O)N(CCO)C1COC1)C3]

100 mg (253 μmol) (7S)-4-[(6-methoxy-1H-indazol-5-yl)amino]-5,6,7,8-tetrahydro[1]benzothieno[2,3-d]pyrimidine-7-carboxylic acid (prepared according to intermediate example 20a) were transformed in analogy to example 1 using 2-(oxetan-3-ylamino)ethanol to give after working up and purification 14.3 mg (11%) of the title compound.

¹H-NMR (DMSO-d6): δ=1.85 (1H), 2.04+2.17 (1H), 2.93 (2H), 3.02-3.68 (7H), 3.98 (3H), 4.49-4.92 (5H), 5.35 (1H), 7.09 (1H), 7.99 (1H), 8.22 (1H), 8.46 (1H), 8.77 (1H), 12.82 (1H) ppm.

### Example 38

### (7S)—N-(2-Hydroxyethyl)-4-[(6-methoxy-1H-indazol-5-yl)amino]-N-methyl-5,6,7,8-tetrahydro[1]benzothieno[2,3-d]pyrimidine-7-carboxamide

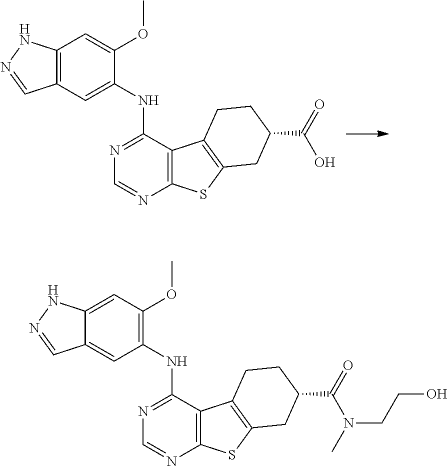 [SMILES: COc1cc2[nH]ncc2cc1Nc1ncnc2sc3c(c12)CC[C@H](C(=O)O)C3 ; COc1cc2[nH]ncc2cc1Nc1ncnc2sc3c(c12)CC[C@H](C(=O)N(C)CCO)C3]

150 mg (379 μmol) (7S)-4-[(6-methoxy-1H-indazol-5-yl)amino]-5,6,7,8-tetrahydro[1]benzothieno[2,3-d]pyrimidine-7-carboxylic acid (prepared according to intermediate example 20a) were transformed in analogy to example 1 using 2-(methylamino)ethanol to give after working up and purification 153 mg (85%) of the title compound.

¹H-NMR (DMSO-d6): δ=1.84 (1H), 2.15 (1H), 2.88+3.14 (3H), 2.92 (2H), 3.10-3.60 (8H), 3.97 (3H), 7.09 (1H), 7.99 (1H), 8.21 (1H), 8.45 (1H), 8.77 (1H), 12.84 (1H) ppm.

### Example 39

### Reference Example

### (7R)—N-(2-Hydroxyethyl)-4-[(6-methoxy-1H-indazol-5-yl)amino]-N-methyl-5,6,7,8-tetrahydro[1]benzothieno[2,3-d]pyrimidine-7-carboxamide

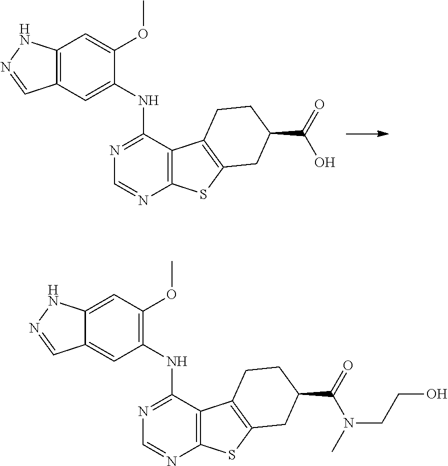 [SMILES: COc1cc2[nH]ncc2cc1Nc1ncnc2sc3c(c12)CC[C@@H](C(=O)O)C3 ; COc1cc2[nH]ncc2cc1Nc1ncnc2sc3c(c12)CC[C@@H](C(=O)N(C)CCO)C3]

150 mg (379 μmol) (7R)-4-[(6-methoxy-1H-indazol-5-yl)amino]-5,6,7,8-tetrahydro[1]benzothieno[2,3-d]pyrimidine-7-carboxylic acid (prepared according to intermediate example 22a) were transformed in analogy to example 1 using 2-(methylamino)ethanol to give after working up and purification 118 mg (65%) of the title compound.

¹H-NMR (DMSO-d6): δ=1.85 (1H), 2.15 (1H), 2.88+3.14 (3H), 2.93 (2H), 3.10-3.60 (8H), 3.98 (3H), 7.09 (1H), 7.99 (1H), 8.22 (1H), 8.45 (1H), 8.77 (1H), 12.84 (1H) ppm.

### Example 40

### (7S)—N-Ethyl-N-(2-hydroxyethyl)-4-[(6-methoxy-1H-indazol-5-yl)amino]-5,6,7,8-tetrahydro[1]benzothieno[2,3-d]pyrimidine-7-carboxamide

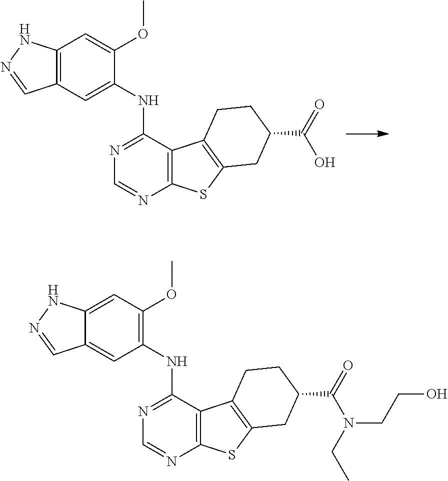 [SMILES: COc1cc2[nH]ncc2cc1Nc1ncnc2sc3c(c12)CC[C@H](C(=O)O)C3 ; CCN(CCO)C(=O)[C@H]1CCc2c(sc3ncnc(Nc4cc5cn[nH]c5cc4OC)c23)C1]

150 mg (379 μmol) (7S)-4-[(6-methoxy-1H-indazol-5-yl)amino]-5,6,7,8-tetrahydro[1]benzothieno[2,3-d]pyrimidine-7-carboxylic acid (prepared according to intermediate example 20a) were transformed in analogy to example 1 using 2-(ethylamino)ethanol to give after working up and purification 81.9 mg (44%) of the title compound.

¹H-NMR (DMSO-d6): δ=1.04+1.15 (3H), 1.85 (1H), 2.13 (1H), 2.84-2.98 (2H), 3.01-3.58 (9H), 3.98 (3H), 4.66+4.83 (1H), 7.09 (1H), 7.99 (1H), 8.22 (1H), 8.46 (1H), 8.78 (1H), 12.84 (1H) ppm.

### Example 41

### Reference Example

### (7R)—N-Ethyl-N-(2-hydroxyethyl)-4-[(6-methoxy-1H-indazol-5-yl)amino]-5,6,7,8-tetrahydro[1]benzothieno[2,3-d]pyrimidine-7-carboxamide

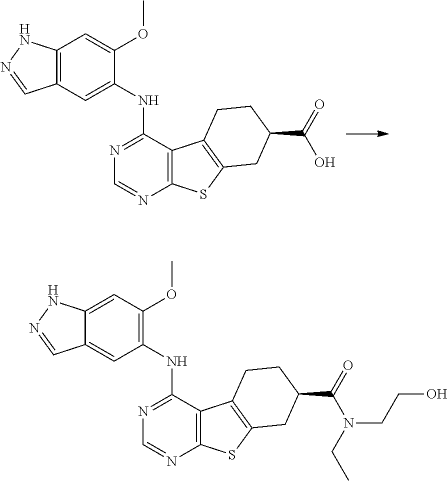 [SMILES: COc1cc2[nH]ncc2cc1Nc1ncnc2sc3c(c12)CC[C@@H](C(=O)O)C3 ; CCN(CCO)C(=O)[C@@H]1CCc2c(sc3ncnc(Nc4cc5cn[nH]c5cc4OC)c23)C1]

150 mg (379 μmol) (7R)-4-[(6-methoxy-1H-indazol-5-yl)amino]-5,6,7,8-tetrahydro[1]benzothieno[2,3-d]pyrimidine-7-carboxylic acid (prepared according to intermediate example 22a) were transformed in analogy to example 1 using 2-(ethylamino)ethanol to give after working up and purification 47.8 mg (26%) of the title compound.

¹H-NMR (DMSO-d6): δ=1.04+1.15 (3H), 1.85 (1H), 2.13 (1H), 2.84-2.98 (2H), 3.01-3.58 (9H), 3.98 (3H), 4.66+4.83 (1H), 7.09 (1H), 7.99 (1H), 8.22 (1H), 8.46 (1H), 8.78 (1H), 12.84 (1H) ppm.

### Example 42

### (7S)—N-(2-Hydroxyethyl)-N-(2-methoxyethyl)-4-[(6-methoxy-1H-indazol-5-yl)amino]-5,6,7,8-tetrahydro[1]benzothieno[2,3-d]pyrimidine-7-carboxamide

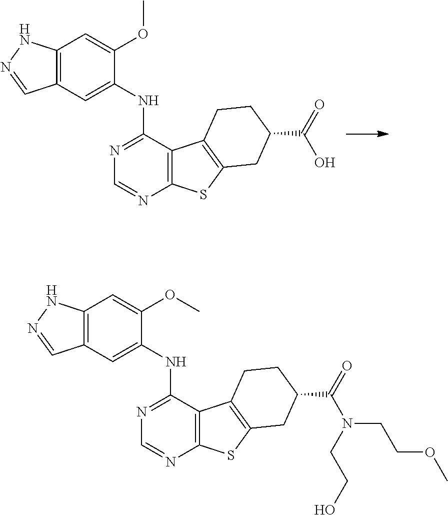 [SMILES: COc1cc2[nH]ncc2cc1Nc1ncnc2sc3c(c12)CC[C@H](C(=O)O)C3 ; COCCN(CCO)C(=O)[C@H]1CCc2c(sc3ncnc(Nc4cc5cn[nH]c5cc4OC)c23)C1]

150 mg (379 μmol) (7S)-4-[(6-methoxy-1H-indazol-5-yl)amino]-5,6,7,8-tetrahydro[1]benzothieno[2,3-d]pyrimidine-7-carboxylic acid (prepared according to intermediate example 20a) were transformed in analogy to example 1 using 2-[(2-methoxyethyl)amino]ethanol to give after working up and purification 144 mg (73%) of the title compound.

¹H-NMR (DMSO-d6): δ=1.85 (1H), 2.13 (1H), 2.91 (2H), 3.13-3.73 (12H), 3.25+3.27 (3H), 3.98 (3H), 7.09 (1H), 7.99 (1H), 8.22 (1H), 8.46 (1H), 8.78 (1H), 12.84 (1H) ppm.

### Example 43

### Reference Example

### (7R)—N-(2-Hydroxyethyl)-N-(2-methoxyethyl)-4-[(6-methoxy-1H-indazol-5-yl)amino]-5,6,7,8-tetrahydro[1]benzothieno[2,3-d]pyrimidine-7-carboxamide

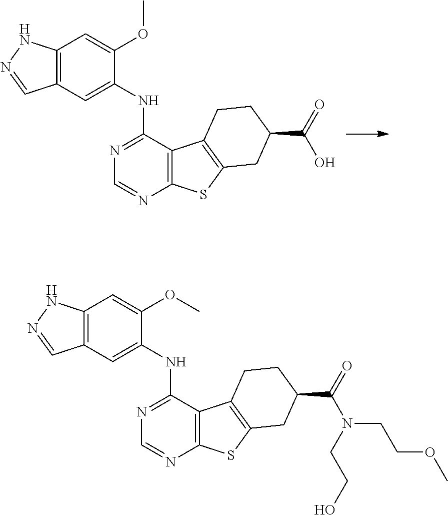 [SMILES: COc1cc2[nH]ncc2cc1Nc1ncnc2sc3c(c12)CC[C@@H](C(=O)O)C3 ; COCCN(CCO)C(=O)[C@@H]1CCc2c(sc3ncnc(Nc4cc5cn[nH]c5cc4OC)c23)C1]

150 mg (379 μmol) (7R)-4-[(6-methoxy-1H-indazol-5-yl)amino]-5,6,7,8-tetrahydro[1]benzothieno[2,3-d]pyrimidine-7-carboxylic acid (prepared according to intermediate example 22a) were transformed in analogy to example 1 using 2-[(2-methoxyethyl)amino]ethanol to give after working up and purification 75.1 mg (38%) of the title compound.

¹H-NMR (DMSO-d6): δ=1.85 (1H), 2.13 (1H), 2.91 (2H), 3.13-3.73 (12H), 3.25+3.27 (3H), 3.98 (3H), 7.09 (1H), 7.99 (1H), 8.22 (1H), 8.46 (1H), 8.78 (1H), 12.84 (1H) ppm.

### Example 44

### [3-(Dimethylamino)azetidin-1-yl]{(7S)-4-[(6-methoxy-1H-indazol-5-yl)amino]-5,6,7,8-tetrahydro[1]benzothieno[2,3-d]pyrimidin-7-yl}methanone

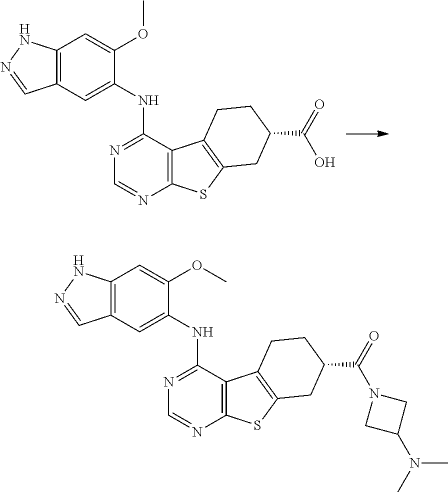 [SMILES: COc1cc2[nH]ncc2cc1Nc1ncnc2sc3c(c12)CC[C@H](C(=O)O)C3 ; COc1cc2[nH]ncc2cc1Nc1ncnc2sc3c(c12)CC[C@H](C(=O)N1CC(N(C)C)C1)C3]

90 mg (228 μmol) (7S)-4-[(6-methoxy-1H-indazol-5-yl)amino]-5,6,7,8-tetrahydro[1]benzothieno[2,3-d]pyrimidine-7-carboxylic acid (prepared according to intermediate example 20a) were transformed in analogy to example 1 using N,N-dimethylazetidin-3-amine to give after working up and purification 58.2 mg (51%) of the title compound.

¹H-NMR (DMSO-d6): δ=1.81 (1H), 2.09 (6H), 2.14 (1H), 2.71-2.96 (3H), 3.00-3.25 (3H), 3.66 (1H), 3.88 (1H), 3.98 (3H), 4.04 (1H), 4.25 (1H), 7.08 (1H), 7.99 (1H), 8.21 (1H), 8.45 (1H), 8.78 (1H), 12.83 (1H) ppm.

### Example 45

### [(3S)-3-(Dimethylamino)pyrrolidin-1-yl]{(7S)-4-[(6-methoxy-1H-indazol-5-yl)amino]-5,6,7,8-tetrahydro[1]benzothieno[2,3-d]pyrimidin-7-yl}methanone

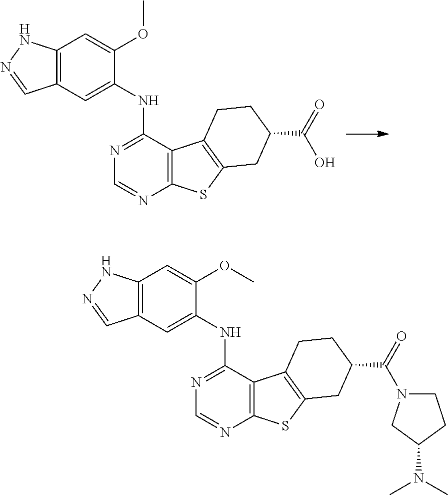 [SMILES: COc1cc2[nH]ncc2cc1Nc1ncnc2sc3c(c12)CC[C@H](C(=O)O)C3 ; COc1cc2[nH]ncc2cc1Nc1ncnc2sc3c(c12)CC[C@H](C(=O)N1CC[C@H](N(C)C)C1)C3]

90 mg (228 μmol) (7S)-4-[(6-methoxy-1H-indazol-5-yl)amino]-5,6,7,8-tetrahydro[1]benzothieno[2,3-d]pyrimidine-7-carboxylic acid (prepared according to intermediate example 20a) were transformed in analogy to example 1 using (3S)—N,N-dimethylpyrrolidin-3-amine to give after working up and purification 20.1 mg (17%) of the title compound.

¹H-NMR (DMSO-d6): δ=1.55-1.91 (2H), 1.96-2.22 (2H), 2.17 (6H), 2.55-3.25 (7H), 3.49-3.87 (3H), 3.98 (3H), 7.09 (1H), 7.99 (1H), 8.21 (1H), 8.46 (1H), 8.78 (1H), 12.83 (1H) ppm.

### Example 46

### [4-(Dimethylamino)piperidin-1-yl]{(7S)-4-[(6-methoxy-1H-indazol-5-yl)amino]-5,6,7,8-tetrahydro[1]benzothieno[2,3-d]pyrimidin-7-yl}methanone

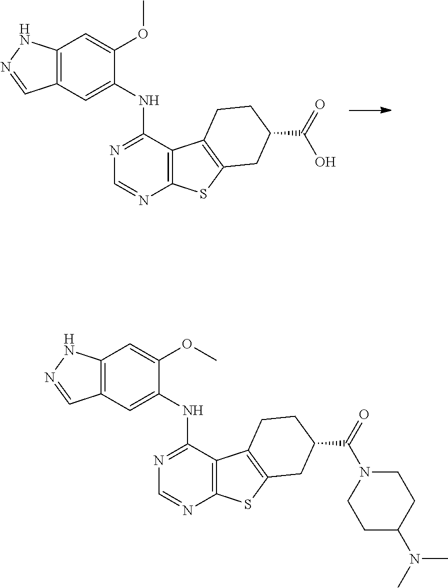 [SMILES: COc1cc2[nH]ncc2cc1Nc1ncnc2sc3c(c12)CC[C@H](C(=O)O)C3 ; COc1cc2[nH]ncc2cc1Nc1ncnc2sc3c(c12)CC[C@H](C(=O)N1CCC(N(C)C)CC1)C3]

100 mg (253 μmol) (7S)-4-[(6-methoxy-1H-indazol-5-yl)amino]-5,6,7,8-tetrahydro[1]benzothieno[2,3-d]pyrimidine-7-carboxylic acid (prepared according to intermediate example 20a) were transformed in analogy to example 1 using N,N-dimethylpiperidin-4-amine to give after working up and purification 46.6 mg (35%) of the title compound.

¹H-NMR (DMSO-d6): δ=1.20 (1H), 1.34 (1H), 1.71-1.94 (3H), 2.11 (1H), 2.17 (6H), 2.32 (1H), 2.61 (1H), 2.82-3.26 (6H), 3.97 (3H), 4.06 (1H), 4.41 (1H), 7.08 (1H), 7.99 (1H), 8.20 (1H), 8.45 (1H), 8.76 (1H), 12.85 (1H) ppm.

### Example 47

### (4-{[2-(Dimethylamino)ethyl](methyl)amino}piperidin-1-yl){(7S)-4-[(6-methoxy-1H-indazol-5-yl)amino]-5,6,7,8-tetrahydro[1]benzothieno[2,3-d]pyrimidin-7-yl}methanone

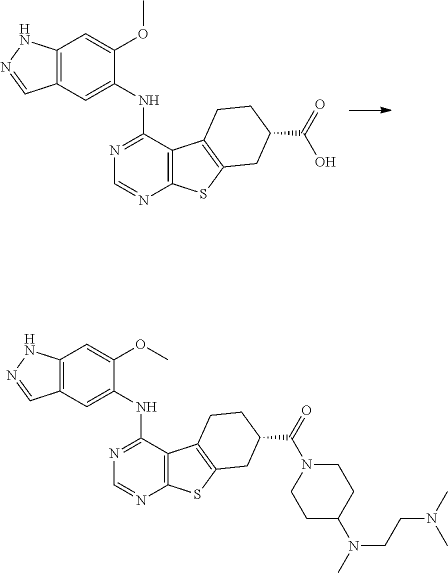 [SMILES: COc1cc2[nH]ncc2cc1Nc1ncnc2sc3c(c12)CC[C@H](C(=O)O)C3 ; COc1cc2[nH]ncc2cc1Nc1ncnc2sc3c(c12)CC[C@H](C(=O)N1CCC(N(C)CCN(C)C)CC1)C3]

90 mg (228 μmol) (7S)-4-[(6-methoxy-1H-indazol-5-yl)amino]-5,6,7,8-tetrahydro[1]benzothieno[2,3-d]pyrimidine-7-carboxylic acid (prepared according to intermediate example 20a) were transformed in analogy to example 1 using

N,N,N′-trimethyl-N′-(piperidin-4-yl)ethane-1,2-diamine to give after working up and purification 78.8 mg (58%) of the title compound.

¹H-NMR (DMSO-d6): δ=1.23 (1H), 1.38 (1H), 1.64-1.93 (3H), 2.07-2.21 (10H), 2.23-2.32 (2H), 2.41-2.64 (4H), 2.83-3.26 (6H), 3.97 (3H), 4.08 (1H), 4.47 (1H), 7.08 (1H), 7.99 (1H), 8.20 (1H), 8.45 (1H), 8.77 (1H), 12.85 (1H) ppm.

### Example 48

### {(7S)-4-[(6-Methoxy-1H-indazol-5-yl)amino]-5,6,7,8-tetrahydro[1]benzothieno[2,3-d]pyrimidin-7-yl}(4-methylpiperazin-1-yl)methanone

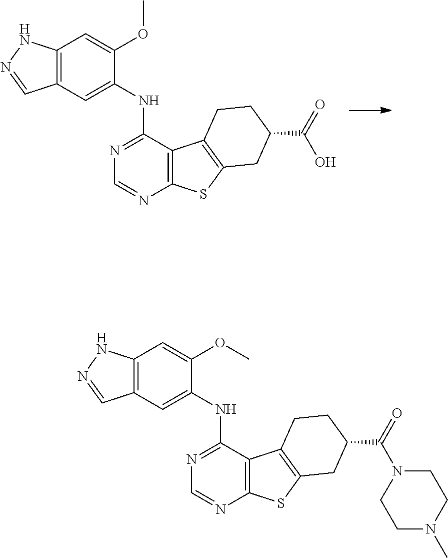 [SMILES: COc1cc2[nH]ncc2cc1Nc1ncnc2sc3c(c12)CC[C@H](C(=O)O)C3 ; COc1cc2[nH]ncc2cc1Nc1ncnc2sc3c(c12)CC[C@H](C(=O)N1CCN(C)CC1)C3]

100 mg (253 μmol) (7S)-4-[(6-methoxy-1H-indazol-5-yl)amino]-5,6,7,8-tetrahydro[1]benzothieno[2,3-d]pyrimidine-7-carboxylic acid (prepared according to intermediate example 20a) were transformed in analogy to example 1 using 1-methylpiperazine to give after working up and purification 47.6 mg (37%) of the title compound.

¹H-NMR (DMSO-d6): δ=1.85 (1H), 2.12 (1H), 2.20 (3H), 2.25-2.38 (4H), 2.85-3.01 (2H), 3.13-3.26 (3H), 3.50 (2H), 3.58 (2H), 3.98 (3H), 7.09 (1H), 7.99 (1H), 8.20 (1H), 8.45 (1H), 8.76 (1H), 12.84 (1H) ppm.

### Example 49

### 4-({(7S)-4-[(6-Methoxy-1H-indazol-5-yl)amino]-5,6,7,8-tetrahydro[1]benzothieno[2,3-d]pyrimidin-7-yl}carbonyl)-N,N-dimethylpiperazine-1-carboxamide

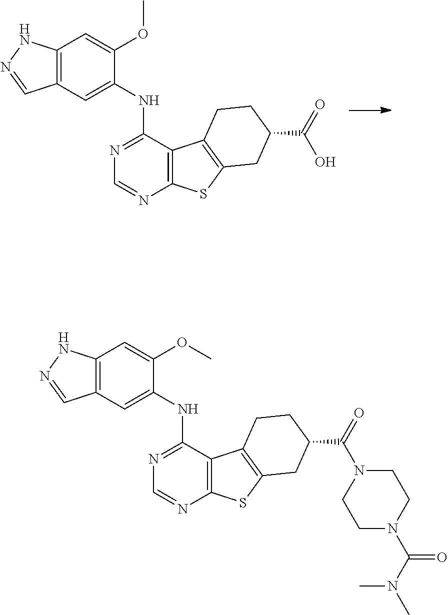 [SMILES: COc1cc2[nH]ncc2cc1Nc1ncnc2sc3c(c12)CC[C@H](C(=O)O)C3 ; COc1cc2[nH]ncc2cc1Nc1ncnc2sc3c(c12)CC[C@H](C(=O)N1CCN(C(=O)N(C)C)CC1)C3]

100 mg (253 μmol) (7S)-4-[(6-methoxy-1H-indazol-5-yl)amino]-5,6,7,8-tetrahydro[1]benzothieno[2,3-d]pyrimidine-7-carboxylic acid (prepared according to intermediate example 20a) were transformed in analogy to example 1 using N,N-dimethylpiperazine-1-carboxamide to give after working up and purification 53.0 mg (37%) of the title compound.

¹H-NMR (DMSO-d6): δ=1.86 (1H), 2.14 (1H), 2.76 (6H), 2.85-2.99 (2H), 3.05-3.28 (7H), 3.52 (2H), 3.61 (2H), 3.97 (3H), 7.08 (1H), 7.99 (1H), 8.20 (1H), 8.45 (1H), 8.77 (1H), 12.85 (1H) ppm.

### Example 50

### {4-[2-(Dimethylamino)ethyl]piperazin-1-yl}{(7S)-4-[(6-methoxy-1H-indazol-5-yl)amino]-5,6,7,8-tetrahydro[1]benzothieno[2,3-d]pyrimidin-7-yl}methanone

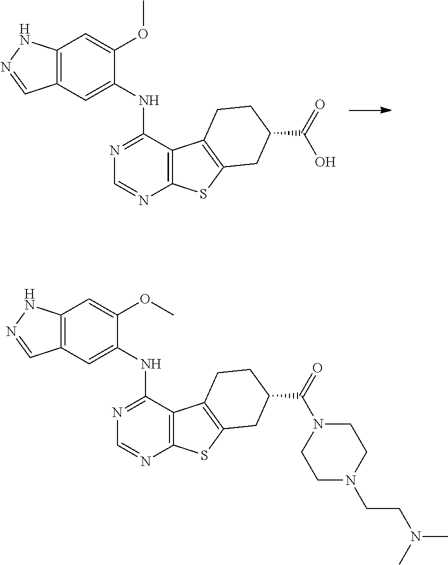 [SMILES: COc1cc2[nH]ncc2cc1Nc1ncnc2sc3c(c12)CC[C@H](C(=O)O)C3 ; COc1cc2[nH]ncc2cc1Nc1ncnc2sc3c(c12)CC[C@H](C(=O)N1CCN(CCN(C)C)CC1)C3]

100 mg (253 μmol) (7S)-4-[(6-methoxy-1H-indazol-5-yl)amino]-5,6,7,8-tetrahydro[1]benzothieno[2,3-d]pyrimidine-7-carboxylic acid (prepared according to intermediate example 20a) were transformed in analogy to example 1 using N,N-dimethyl-2-(piperazin-1-yl)ethanamine to give after working up and purification 70.3 mg (49%) of the title compound.

¹H-NMR (DMSO-d6): δ=1.84 (1H), 2.10 (1H), 2.13 (6H), 2.28-2.47 (8H), 2.82-3.01 (2H), 3.10-3.26 (3H), 3.49 (2H), 3.57 (2H), 3.97 (3H), 7.08 (1H), 7.99 (1H), 8.20 (1H), 8.45 (1H), 8.76 (1H), 12.85 (1H) ppm.

### Example 51

### (7S)—N-[2-(Dimethylamino)ethyl]-4-[(6-methoxy-1H-indazol-5-yl)amino]-N-methyl-5,6,7,8-tetrahydro[1]benzothieno[2,3-d]pyrimidine-7-carboxamide

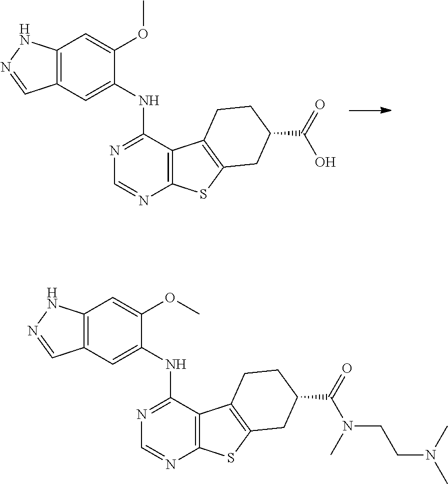 [SMILES: COc1cc2[nH]ncc2cc1Nc1ncnc2sc3c(c12)CC[C@H](C(=O)O)C3 ; COc1cc2[nH]ncc2cc1Nc1ncnc2sc3c(c12)CC[C@H](C(=O)N(C)CCN(C)C)C3]

100 mg (253 μmol) (7S)-4-[(6-methoxy-1H-indazol-5-yl)amino]-5,6,7,8-tetrahydro[1]benzothieno[2,3-d]pyrimidine-7-carboxylic acid (prepared according to intermediate example 20a) were transformed in analogy to example 1 using N,N,N′-trimethylethane-1,2-diamine to give after working up and purification 61.4 mg (48%) of the title compound.

¹H-NMR (DMSO-d6): δ=1.83 (1H), 2.13 (1H), 2.16+2.18 (6H), 2.34+2.42 (2H), 2.87+3.10 (3H), 2.91 (2H), 3.11-3.54 (5H), 3.97 (3H), 7.09 (1H), 7.99 (1H), 8.22 (1H), 8.46 (1H), 8.77 (1H), 12.86 (1H) ppm.

### Example 52

### (7S)—N-[2-(Dimethylamino)-2-oxoethyl]-4-[(6-methoxy-1H-indazol-5-yl)amino]-N-methyl-5,6,7,8-tetrahydro[1]benzothieno[2,3-d]pyrimidine-7-carboxamide

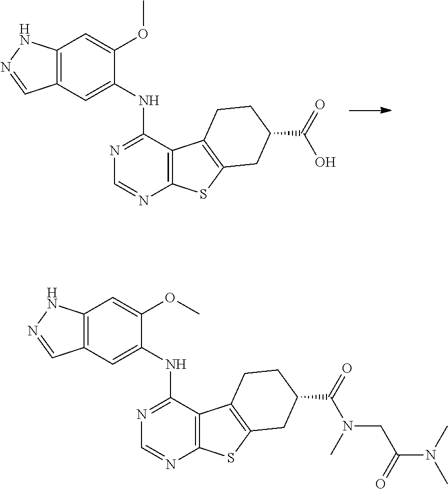 [SMILES: COc1cc2[nH]ncc2cc1Nc1ncnc2sc3c(c12)CC[C@H](C(=O)O)C3 ; COc1cc2[nH]ncc2cc1Nc1ncnc2sc3c(c12)CC[C@H](C(=O)N(C)CC(=O)N(C)C)C3]

90 mg (228 μmol) (7S)-4-[(6-methoxy-1H-indazol-5-yl)amino]-5,6,7,8-tetrahydro[1]benzothieno[2,3-d]pyrimidine-7-carboxylic acid (prepared according to intermediate example 20a) were transformed in analogy to example 1 using N,N,N²-trimethylglycinamide to give after working up and purification 71.5 mg (60%) of the title compound.

¹H-NMR (DMSO-d6): δ=1.85 (1H), 2.08+2.20 (1H), 2.78-2.98 (10H), 3.06-3.26 (4H), 3.96+3.99 (3H), 4.04-4.40 (2H), 7.09 (1H), 7.99 (1H), 8.19+8.22 (1H), 8.45 (1H), 8.75+8.79 (1H), 12.83 (1H) ppm.

### Example 53

### (7S)—N-[2-(Dimethylamino)ethyl]-N-ethyl-4-[(6-methoxy-1H-indazol-5-yl)amino]-5,6,7,8-tetrahydro[1]benzothieno[2,3-d]pyrimidine-7-carboxamide

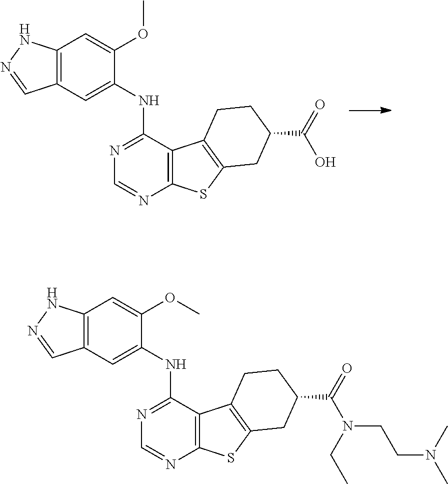 [SMILES: COc1cc2[nH]ncc2cc1Nc1ncnc2sc3c(c12)CC[C@H](C(=O)O)C3 ; CCN(CCN(C)C)C(=O)[C@H]1CCc2c(sc3ncnc(Nc4cc5cn[nH]c5cc4OC)c23)C1]

100 mg (253 μmol) (7S)-4-[(6-methoxy-1H-indazol-5-yl)amino]-5,6,7,8-tetrahydro[1]benzothieno[2,3-d]pyrimidine-7-carboxylic acid (prepared according to intermediate example 20a) were transformed in analogy to example 1 using N′-ethyl-N,N-dimethylethane-1,2-diamine to give after working up and purification 51.2 mg (39%) of the title compound.

¹H-NMR (DMSO-d6): δ=1.04+1.15 (3H), 1.86 (1H), 2.11 (1H), 2.16+2.18 (6H), 2.33+2.41 (2H), 2.83-3.53 (9H), 3.98 (3H), 7.08 (1H), 7.99 (1H), 8.21 (1H), 8.46 (1H), 8.78 (1H), 12.85 (1H) ppm.

### Example 54

### (7S)—N-[3-(Dimethylamino)propyl]-4-[(6-methoxy-1H-indazol-5-yl)amino]-N-methyl-5,6,7,8-tetrahydro[1]benzothieno[2,3-d]pyrimidine-7-carboxamide

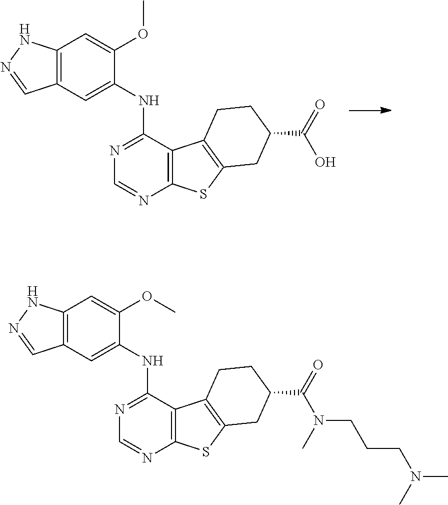 [SMILES: COc1cc2[nH]ncc2cc1Nc1ncnc2sc3c(c12)CC[C@H](C(=O)O)C3 ; COc1cc2[nH]ncc2cc1Nc1ncnc2sc3c(c12)CC[C@H](C(=O)N(C)CCCN(C)C)C3]

100 mg (253 μmol) (7S)-4-[(6-methoxy-1H-indazol-5-yl)amino]-5,6,7,8-tetrahydro[1]benzothieno[2,3-d]pyrimidine-7-carboxylic acid (prepared according to intermediate example 20a) were transformed in analogy to example 1 using N′-ethyl-N,N-dimethylethane-1,2-diamine to give after working up and purification 28.1 mg (20%) of the title compound.

¹H-NMR (DMSO-d6): δ=1.54-1.92 (3H), 2.05-2.26 (9H), 2.85+3.09 (3H), 2.91 (2H), 3.11-3.49 (5H), 3.96+3.97 (3H), 7.08 (1H), 7.99 (1H), 8.20 (1H), 8.45 (1H), 8.77+8.78 (1H), 12.86 (1H) ppm.

### Example 55

### (7S)—N-[3-(Dimethylamino)-3-oxopropyl]-4-[(6-methoxy-1H-indazol-5-yl)amino]-N-methyl-5,6,7,8-tetrahydro[1]benzothieno[2,3-d]pyrimidine-7-carboxamide

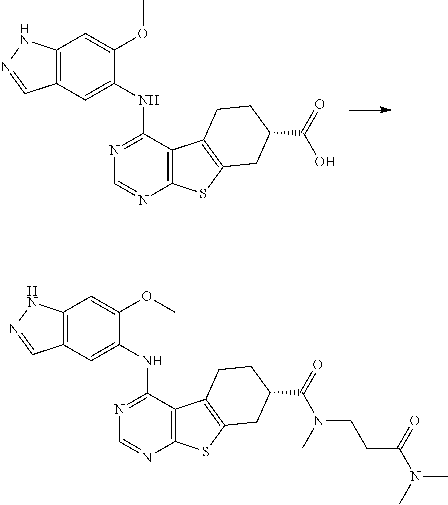 [SMILES: COc1cc2[nH]ncc2cc1Nc1ncnc2sc3c(c12)CC[C@H](C(=O)O)C3 ; COc1cc2[nH]ncc2cc1Nc1ncnc2sc3c(c12)CC[C@H](C(=O)N(C)CCC(=O)N(C)C)C3]

100 mg (253 μmol) (7S)-4-[(6-methoxy-1H-indazol-5-yl)amino]-5,6,7,8-tetrahydro[1]benzothieno[2,3-d]pyrimidine-7-carboxylic acid (prepared according to intermediate example 20a) were transformed in analogy to example 1 using N,N,N³-trimethyl-beta-alaninamide to give after working up and purification 42.4 mg (31%) of the title compound.

¹H-NMR (DMSO-d6): δ=1.83 (1H), 2.13 (1H), 2.52+2.65 (2H), 2.80 (3H), 2.86+3.11 (3H), 2.92 (2H), 2.97 (3H), 3.13-3.73 (5H), 3.97 (3H), 7.08 (1H), 7.99 (1H), 8.21 (1H), 8.45 (1H), 8.77+8.78 (1H), 12.85 (1H) ppm.

### Example 56

### (7S)—N-Cyclopropyl-N-[3-(dimethylamino)-3-oxopropyl]-4-[(6-methoxy-1H-indazol-5-yl)amino]-5,6,7,8-tetrahydro[1]benzothieno[2,3-d]pyrimidine-7-carboxamide

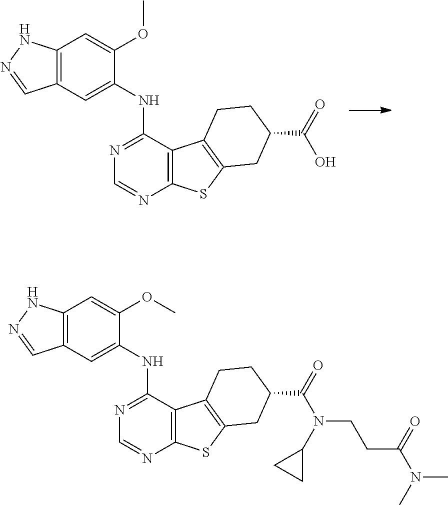 [SMILES: COc1cc2[nH]ncc2cc1Nc1ncnc2sc3c(c12)CC[C@H](C(=O)O)C3 ; COc1cc2[nH]ncc2cc1Nc1ncnc2sc3c(c12)CC[C@H](C(=O)N(CCC(=O)N(C)C)C1CC1)C3]

90 mg (228 μmol) (7S)-4-[(6-methoxy-1H-indazol-5-yl)amino]-5,6,7,8-tetrahydro[1]benzothieno[2,3-d]pyrimidine-7-carboxylic acid (prepared according to intermediate example 20a) were transformed in analogy to example 1 using N³-cyclopropyl-N,N-dimethyl-beta-alaninamide to give after working up and purification 17.8 mg (14%) of the title compound.

¹H-NMR (DMSO-d6): δ=0.81 (2H), 0.89 (2H), 1.84 (1H), 2.19 (1H), 2.54 (2H), 2.80 (3H), 2.87 (1H), 2.95 (2H), 2.98 (3H), 3.17 (1H), 3.28 (1H), 3.42-3.62 (3H), 3.97 (3H), 7.09 (1H), 7.99 (1H), 8.21 (1H), 8.46 (1H), 8.76 (1H), 12.84 (1H) ppm.

### Example 57

### (7S)—N-(2-{[2-(Dimethylamino)ethyl](methyl)amino}ethyl)-4-[(6-methoxy-1H-indazol-5-yl)amino]-N-methyl-5,6,7,8-tetrahydro[1]benzothieno[2,3-d]pyrimidine-7-carboxamide

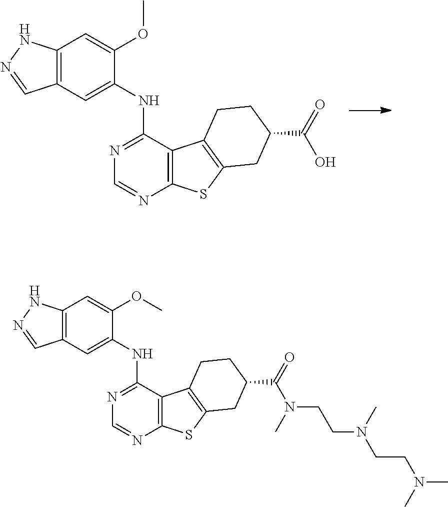 [SMILES: COc1cc2[nH]ncc2cc1Nc1ncnc2sc3c(c12)CC[C@H](C(=O)O)C3 ; COc1cc2[nH]ncc2cc1Nc1ncnc2sc3c(c12)CC[C@H](C(=O)N(C)CCN(C)CCN(C)C)C3]

90 mg (228 μmol) (7S)-4-[(6-methoxy-1H-indazol-5-yl)amino]-5,6,7,8-tetrahydro[1]benzothieno[2,3-d]pyrimidine-7-carboxylic acid (prepared according to intermediate example 20a) were transformed in analogy to example 1 using N,N,N′-trimethyl-N′-[2-(methylamino)ethyl]ethane-1,2-diamine to give after working up and purification 73.5 mg (57%) of the title compound.

¹H-NMR (DMSO-d6): δ=1.84 (1H), 2.05 (3H), 2.12 (3H), 2.20 (3H), 2.08-2.34 (4H), 2.37-2.46 (4H), 2.87+3.11 (3H), 3.07-3.56 (6H), 3.98 (3H), 7.09 (1H), 7.99 (1H), 8.21 (1H), 8.46 (1H), 8.78 (1H), 12.84 (1H) ppm.

### Example 58

### (7S)—N-[3-(Dimethylamino)propyl]-N-(2-hydroxyethyl)-4-[(6-methoxy-1H-indazol-5-yl)amino]-5,6,7,8-tetrahydro[1]benzothieno[2,3-d]pyrimidine-7-carboxamide

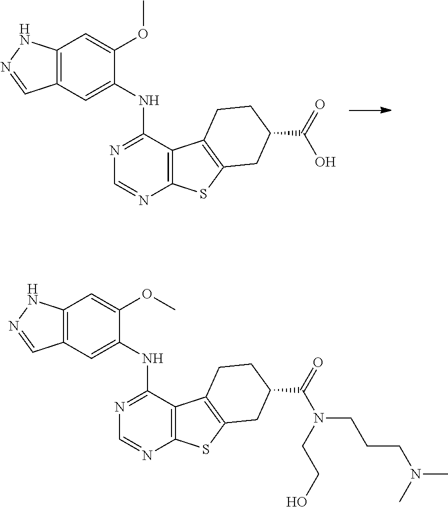 [SMILES: COc1cc2[nH]ncc2cc1Nc1ncnc2sc3c(c12)CC[C@H](C(=O)O)C3 ; COc1cc2[nH]ncc2cc1Nc1ncnc2sc3c(c12)CC[C@H](C(=O)N(CCO)CCCN(C)C)C3]

90 mg (228 μmol) (7S)-4-[(6-methoxy-1H-indazol-5-yl)amino]-5,6,7,8-tetrahydro[1]benzothieno[2,3-d]pyrimidine-7-carboxylic acid (prepared according to intermediate example 20a) were transformed in analogy to example 1 using 2-{[3-(dimethylamino)propyl]amino}ethanol to give after working up and purification 47.5 mg (38%) of the title compound.

¹H-NMR (DMSO-d6): δ=1.56-1.74 (2H), 1.86 (1H), 2.07-2.22 (9H), 2.86-3.02 (2H), 3.11-3.56 (9H), 3.98 (3H), 4.66+4.84 (1H), 7.09 (1H), 7.99 (1H), 8.22 (1H), 8.46 (1H), 8.78 (1H), 12.84 (1H) ppm.

### Example 59

### (7S)—N,N-bis[3-(Dimethylamino)propyl]-4-[(6-methoxy-1H-indazol-5-yl)amino]-5,6,7,8-tetrahydro[1]benzothieno[2,3-d]pyrimidine-7-carboxamide

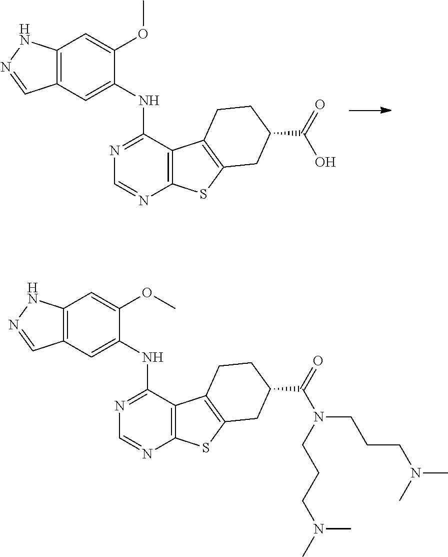 [SMILES: COc1cc2[nH]ncc2cc1Nc1ncnc2sc3c(c12)CC[C@H](C(=O)O)C3 ; COc1cc2[nH]ncc2cc1Nc1ncnc2sc3c(c12)CC[C@H](C(=O)N(CCCN(C)C)CCCN(C)C)C3]

90 mg (228 μmol) (7S)-4-[(6-methoxy-1H-indazol-5-yl)amino]-5,6,7,8-tetrahydro[1]benzothieno[2,3-d]pyrimidine-7-carboxylic acid (prepared according to intermediate example 20a) were transformed in analogy to example 1 using N′-[3-(dimethylamino)propyl]-N,N-dimethylpropane-1,3-diamine to give after working up and purification 64.3 mg (48%) of the title compound.

¹H-NMR (DMSO-d6): δ=1.47-1.78 (4H), 1.87 (1H), 2.01-2.27 (17H), 2.82-3.03 (2H), 3.06-3.48 (7H), 3.97 (3H), 7.08 (1H), 7.99 (1H), 8.21 (1H), 8.46 (1H), 8.78 (1H), 12.86 (1H) ppm.

### Example 60

### (7S)—N-[2-(Dimethylamino)ethyl]-4-[(6-methoxy-1H-indazol-5-yl)amino]-N-(pyridin-2-ylmethyl)-5,6,7,8-tetrahydro[1]benzothieno[2,3-d]pyrimidine-7-carboxamide

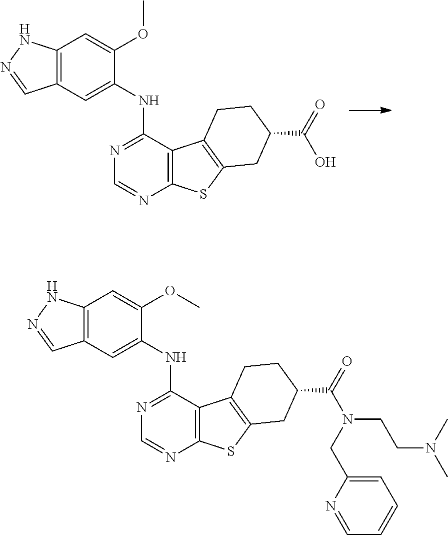 [SMILES: COc1cc2[nH]ncc2cc1Nc1ncnc2sc3c(c12)CC[C@H](C(=O)O)C3 ; COc1cc2[nH]ncc2cc1Nc1ncnc2sc3c(c12)CC[C@H](C(=O)N(CCN(C)C)Cc1ccccn1)C3]

100 mg (253 μmol) (7S)-4-[(6-methoxy-1H-indazol-5-yl)amino]-5,6,7,8-tetrahydro[1]benzothieno[2,3-d]pyrimidine-7-carboxylic acid (prepared according to intermediate example 20a) were transformed in analogy to example 1 using N,N-dimethyl-N′-(pyridin-2-ylmethyl)ethane-1,2-diamine to give after working up and purification 94.5 mg (64%) of the title compound.

¹H-NMR (DMSO-d6): δ=1.71-2.00 (2H), 2.26-3.32 (7H), 2.34+2.57 (6H), 3.49-3.80 (2H), 3.95+3.99 (3H), 4.54-4.94 (2H), 7.09 (1H), 7.22-7.46 (2H), 7.78+7.85 (1H), 7.99 (1H), 8.16+8.25 (1H), 8.44+8.46 (1H), 8.52+8.61 (1H), 8.76+8.78 (1H) ppm.

### Example 61

### (7S)—N-[2-(Dimethylamino)ethyl]-4-[(6-methoxy-1H-indazol-5-yl)amino]-N-(pyridin-3-ylmethyl)-5,6,7,8-tetrahydro[1]benzothieno[2,3-d]pyrimidine-7-carboxamide

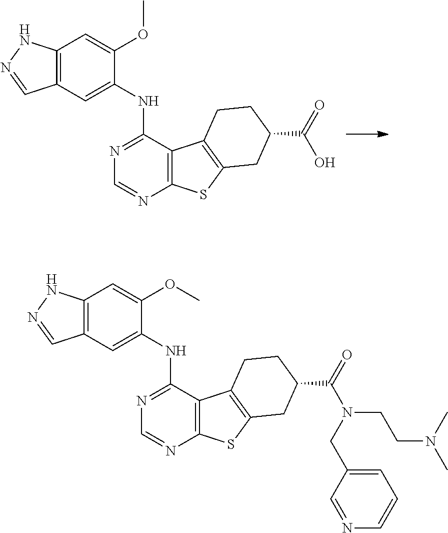 [SMILES: COc1cc2[nH]ncc2cc1Nc1ncnc2sc3c(c12)CC[C@H](C(=O)O)C3 ; COc1cc2[nH]ncc2cc1Nc1ncnc2sc3c(c12)CC[C@H](C(=O)N(CCN(C)C)Cc1cccnc1)C3]

100 mg (253 μmol) (7S)-4-[(6-methoxy-1H-indazol-5-yl)amino]-5,6,7,8-tetrahydro[1]benzothieno[2,3-d]pyrimidine-7-carboxylic acid (prepared according to intermediate example 20a) were transformed in analogy to example 1 using N,N-dimethyl-N′-(pyridin-3-ylmethyl)ethane-1,2-diamine to give after working up and purification 47.1 mg (32%) of the title compound.

¹H-NMR (DMSO-d6): δ=1.75-2.08 (2H), 2.15 (6H), 2.22 (1H), 2.41 (2H), 2.94-3.57 (6H), 3.97+3.98 (3H), 4.52-4.89 (2H), 7.09 (1H), 7.34-7.45 (1H), 7.62-7.72 (1H), 7.99 (1H), 8.17+8.23 (1H), 8.43-8.56 (3H), 8.76+8.79 (1H), 12.85 (1H) ppm.

### Example 62

### (7S)—N-[2-(Dimethylamino)ethyl]-4-[(6-methoxy-1H-indazol-5-yl)amino]-N-(pyridin-4-ylmethyl)-5,6,7,8-tetrahydro[1]benzothieno[2,3-d]pyrimidine-7-carboxamide

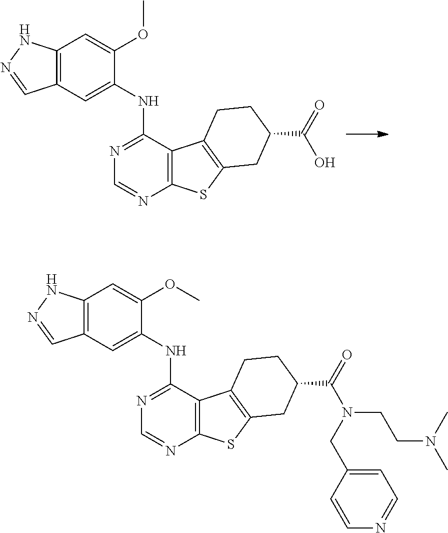 [SMILES: COc1cc2[nH]ncc2cc1Nc1ncnc2sc3c(c12)CC[C@H](C(=O)O)C3 ; COc1cc2[nH]ncc2cc1Nc1ncnc2sc3c(c12)CC[C@H](C(=O)N(CCN(C)C)Cc1ccncc1)C3]

100 mg (253 μmol) (7S)-4-[(6-methoxy-1H-indazol-5-yl)amino]-5,6,7,8-tetrahydro[1]benzothieno[2,3-d]pyrimidine-7-carboxylic acid (prepared according to intermediate example 20a) were transformed in analogy to example 1 using N,N-dimethyl-N′-(pyridin-4-ylmethyl)ethane-1,2-diamine to give after working up and purification 5.5 mg (4%) of the title compound.

¹H-NMR (DMSO-d6): δ=1.75-2.05 (2H), 2.15 (6H), 2.27 (1H), 2.42 (2H), 2.79-3.60 (6H), 3.95+3.98 (3H), 4.53-4.90 (2H), 7.07+7.09 (1H), 7.18-7.33 (2H), 7.99 (1H), 8.14+8.23 (1H), 8.43+8.46 (1H), 8.48-8.60 (2H), 8.75+8.79 (1H), 12.85 (1H) ppm.

### Example 63

### (7S)—N-Benzyl-N-[3-(dimethylamino)propyl]-4-[(6-methoxy-1H-indazol-5-yl)amino]-5,6,7,8-tetrahydro[1]benzothieno[2,3-d]pyrimidine-7-carboxamide

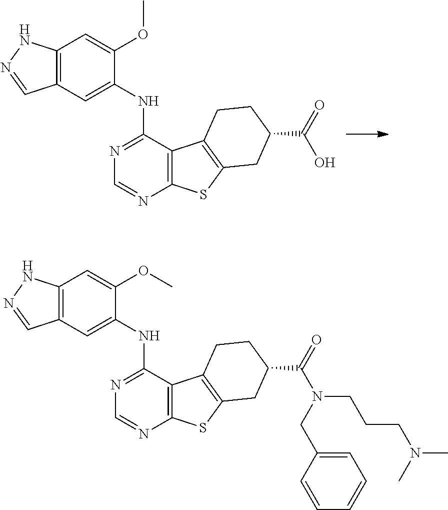 [SMILES: COc1cc2[nH]ncc2cc1Nc1ncnc2sc3c(c12)CC[C@H](C(=O)O)C3 ; COc1cc2[nH]ncc2cc1Nc1ncnc2sc3c(c12)CC[C@H](C(=O)N(CCCN(C)C)Cc1ccccc1)C3]

90 mg (228 μmol) (7S)-4-[(6-methoxy-1H-indazol-5-yl)amino]-5,6,7,8-tetrahydro[1]benzothieno[2,3-d]pyrimidine-7-carboxylic acid (prepared according to intermediate example 20a) were transformed in analogy to example 1 using N′-benzyl-N,N-dimethylpropane-1,3-diamine to give after working up and purification 89.5 mg (66%) of the title compound.

¹H-NMR (DMSO-d6): δ=1.56-1.81 (2H), 1.80-2.13 (2H), 2.05+2.08 (6H), 2.17 (2H), 2.76-3.47 (7H), 3.96+3.98 (3H), 4.44-4.85 (2H), 7.07+7.09 (1H), 7.20-7.42 (5H), 7.98+7.99 (1H), 8.16+8.23 (1H), 8.44+8.47 (1H), 8.76+8.80 (1H), 12.85 (1H) ppm.

### Example 64

### (4S,5R)-3-({(7S)-4-[(6-Methoxy-1H-indazol-5-yl)amino]-5,6,7,8-tetrahydro[1]benzothieno[2,3-d]pyrimidin-7-yl}carbonyl)-4-methyl-5-phenyl-1,3-oxazolidin-2-one

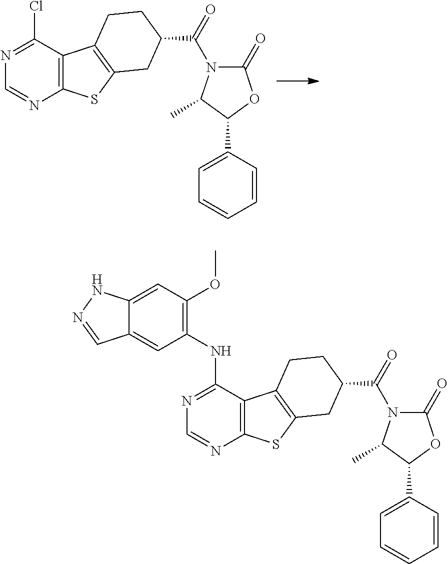

23 mg (54 μmol) (4S,5R)-3-{[(7S)-4-chloro-5,6,7,8-tetrahydro[1]benzothieno[2,3-d]pyrimidin-7-yl]carbonyl}-4-methyl-5-phenyl-1,3-oxazolidin-2-one (prepared according to intermediate example 1d) were transformed in analogy to intermediate example 1b using 6-methoxy-1H-indazol-5-amine to give after working up and purification 2.9 mg (9%) of the title compound.

¹H-NMR (DMSO-d6): δ=0.80 (3H), 1.93 (1H), 2.37 (1H), 3.06 (2H), 3.24 (2H), 3.96 (4H), 4.88 (1H), 5.90 (1H), 7.09 (1H), 7.37-7.47 (5H), 7.99 (1H), 8.22 (1H), 8.46 (1H), 8.73 (1H), 12.84 (1H) ppm.

### Example 65

### (7S)—N-(2,2-Difluoroethyl)-4-[(6-ethoxy-1H-indazol-5-yl)amino]-N-methyl-5,6,7,8-tetrahydro[1]benzothieno[2,3-d]pyrimidine-7-carboxamide

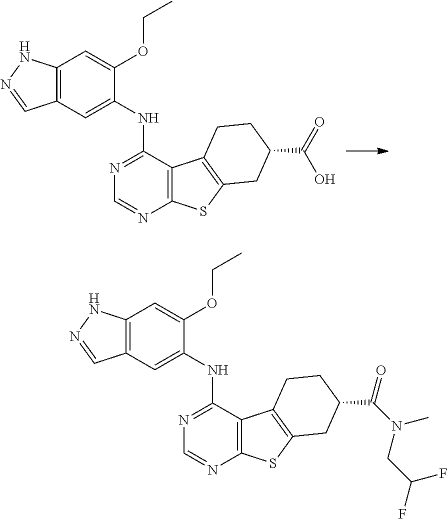 [SMILES: CCOc1cc2[nH]ncc2cc1Nc1ncnc2sc3c(c12)CC[C@H](C(=O)O)C3 ; CCOc1cc2[nH]ncc2cc1Nc1ncnc2sc3c(c12)CC[C@H](C(=O)N(C)CC(F)F)C3]

150 mg (366 μmol) (7S)-4-[(6-ethoxy-1H-indazol-5-yl)amino]-5,6,7,8-tetrahydro[1]benzothieno[2,3-d]pyrimidine-7-carboxylic acid (prepared according to intermediate example 65a) were transformed in analogy to example 1 using 2,2-difluoro-N-methylethanamine to give after working up and purification 70.8 mg (38%) of the title compound.

¹H-NMR (DMSO-d6): δ=1.46 (3H), 1.87 (1H), 2.07 (1H), 2.85-3.02 (2H), 2.95+3.19 (3H), 3.16-3.29 (2H), 3.63-4.05 (2H), 4.20 (2H), 5.95 (1H), 6.13+6.28 (1H), 7.04 (1H), 7.99 (1H), 8.34 (1H), 8.52 (1H), 9.02 (1H), 12.82 (1H) ppm.

### Example 65a

### (7S)-4-[(6-Ethoxy-1H-indazol-5-yl)amino]-5,6,7,8-tetrahydro[1]benzothieno[2,3-d]pyrimidine-7-carboxylic acid

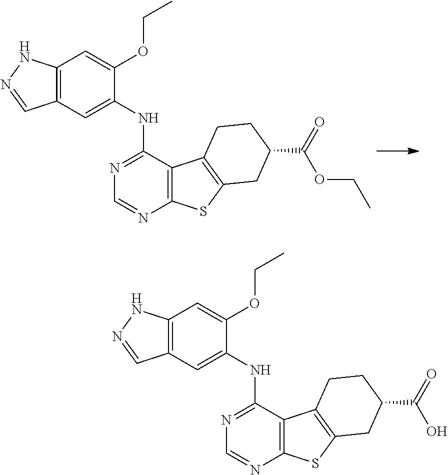 [SMILES: CCOC(=O)[C@H]1CCc2c(sc3ncnc(Nc4cc5cn[nH]c5cc4OCC)c23)C1 ; CCOc1cc2[nH]ncc2cc1Nc1ncnc2sc3c(c12)CC[C@H](C(=O)O)C3]

328 mg (750 μmol) ethyl (7S)-4-[(6-ethoxy-1H-indazol-5-yl)amino]-5,6,7,8-tetrahydro[1]benzothieno[2,3-d]pyrimidine-7-carboxylate (prepared according to intermediate example 65b) were transformed in analogy to intermediate example 1a to give after working up and purification 264 mg (82%) of the title compound.

### Example 65b

### Ethyl (7S)-4-[(6-ethoxy-1H-indazol-5-yl)amino]-5,6,7,8-tetrahydro[1]benzothieno[2,3-d]pyrimidine-7-carboxylate

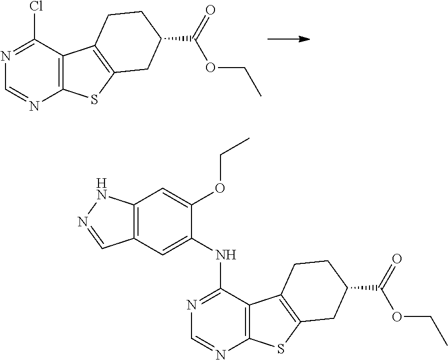

300 mg (1.01 mmol) ethyl (7S)-4-chloro-5,6,7,8-tetrahydro[1]benzothieno[2,3-d]pyrimidine-7-carboxylate (prepared according to intermediate example 1c) were transformed in analogy to intermediate example 1b using 6-ethoxy-1H-indazol-5-amine (prepared according to intermediate example 65c) to give after working up and purification 333 mg (68%) of the title compound.

### Example 65c

### 6-Ethoxy-1H-indazol-5-amine

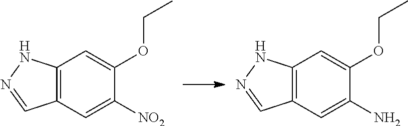

10.0 g (48.3 mmol) 6-ethoxy-5-nitro-1H-indazole (Supplier: Angene Chemicals, Hong Kong PO#2343258 [symbol] 2374166) were transformed in analogy to intermediate example 94b to give after working up and purification 5.08 g (59%) of the title compound.

### Example 66

### (7S)-4-[(6-Ethoxy-1H-indazol-5-yl)amino]-N-methyl-N-(3,3,3-trifluoropropyl)-5,6,7,8-tetrahydro[1]benzothieno[2,3-d]pyrimidine-7-carboxamide

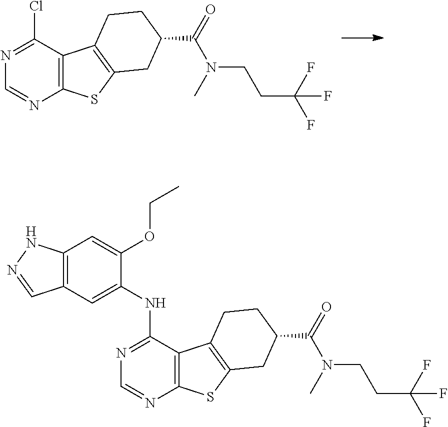 [SMILES: CN(CCC(F)(F)F)C(=O)[C@H]1CCc2c(sc3ncnc(Cl)c23)C1 ; CCOc1cc2[nH]ncc2cc1Nc1ncnc2sc3c(c12)CC[C@H](C(=O)N(C)CCC(F)(F)F)C3]

75 mg (199 μmol) (7S)-4-chloro-N-methyl-N-(3,3,3-trifluoropropyl)-5,6,7,8-tetrahydro[1]benzothieno[2,3-d]pyrimidine-7-carboxamide (prepared according to intermediate example 66a) were transformed in analogy to intermediate example 1b using 6-ethoxy-1H-indazol-5-amine to give after working up and purification 55 mg (53%) of the title compound.

¹H-NMR (DMSO-d6): δ=1.46 (3H), 1.85 (1H), 2.07 (1H), 2.45-2.58 (2H), 2.87+3.12 (3H), 2.88-3.00 (2H), 3.08-3.28 (3H), 3.47-3.73 (2H), 4.21 (2H), 7.05 (1H), 7.99 (1H), 8.34 (1H), 8.52 (1H), 9.01 (1H), 12.80 (1H) ppm.

### Example 66a

### (7S)-4-Chloro-N-methyl-N-(3,3,3-trifluoropropyl)-5,6,7,8-tetrahydro[1]benzothieno[2,3-d]pyrimidine-7-carboxamide

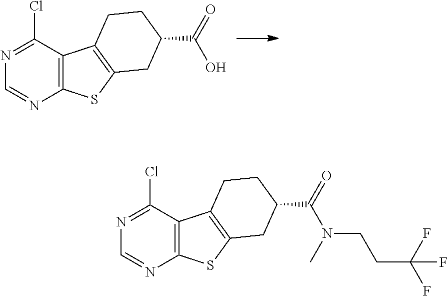 [SMILES: O=C(O)[C@H]1CCc2c(sc3ncnc(Cl)c23)C1 ; CN(CCC(F)(F)F)C(=O)[C@H]1CCc2c(sc3ncnc(Cl)c23)C1]

150 mg (558 μmol) (7S)-4-chloro-5,6,7,8-tetrahydro[1]benzothieno[2,3-d]pyrimidine-7-carboxylic acid (prepared according to intermediate example 66b) were transformed in analogy to example 1 using 3,3,3-trifluoro-N-methylpropan-1-amine to give after working up and purification 155 mg (74%) of the title compound.

### Example 66b

### (7S)-4-Chloro-5,6,7,8-tetrahydro[1]benzothieno[2,3-d]pyrimidine-7-carboxylic acid

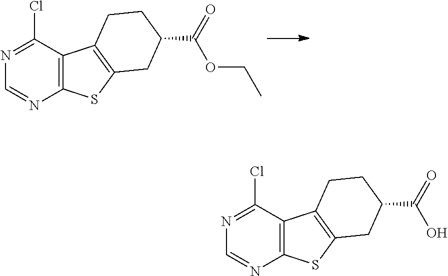 [SMILES: CCOC(=O)[C@H]1CCc2c(sc3ncnc(Cl)c23)C1 ; O=C(O)[C@H]1CCc2c(sc3ncnc(Cl)c23)C1]

4.38 g (14.76 mmol) ethyl (7S)-4-chloro-5,6,7,8-tetrahydro[1]benzothieno[2,3-d]pyrimidine-7-carboxylate (prepared according to intermediate example 1c) were transformed in analogy to intermediate example 1a to give after working up and purification 3.87 g (93%) of the title compound.

### Example 67

### {(7S)-4-[(6-Ethoxy-1H-indazol-5-yl)amino]-5,6,7,8-tetrahydro[1]benzothieno[2,3-d]pyrimidin-7-yl}(pyrrolidin-1-yl)methanone

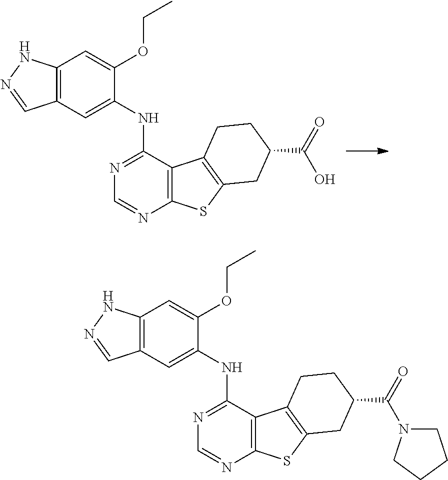 [SMILES: CCOc1cc2[nH]ncc2cc1Nc1ncnc2sc3c(c12)CC[C@H](C(=O)O)C3 ; CCOc1cc2[nH]ncc2cc1Nc1ncnc2sc3c(c12)CC[C@H](C(=O)N1CCCC1)C3]

100 mg (244 μmol) (7S)-4-[(6-ethoxy-1H-indazol-5-yl)amino]-5,6,7,8-tetrahydro[1]benzothieno[2,3-d]pyrimidine-7-carboxylic acid (prepared according to intermediate example 65a) were transformed in analogy to example 1 using pyrrolidine to give after working up and purification 96.9 mg (81%) of the title compound.

¹H-NMR (DMSO-d6): δ=1.46 (3H), 1.75-1.95 (5H), 2.10 (1H), 2.88-3.01 (3H), 3.16 (1H), 3.24-3.37 (3H), 3.49-3.61 (2H), 4.19 (2H), 7.04 (1H), 7.98 (1H), 8.33 (1H), 8.51 (1H), 9.01 (1H), 12.80 (1H) ppm.

### Example 68

### 1-({(7S)-4-[(6-Ethoxy-1H-indazol-5-yl)amino]-5,6,7,8-tetrahydro[1]benzothieno[2,3-d]pyrimidin-7-yl}carbonyl)piperidin-4-one

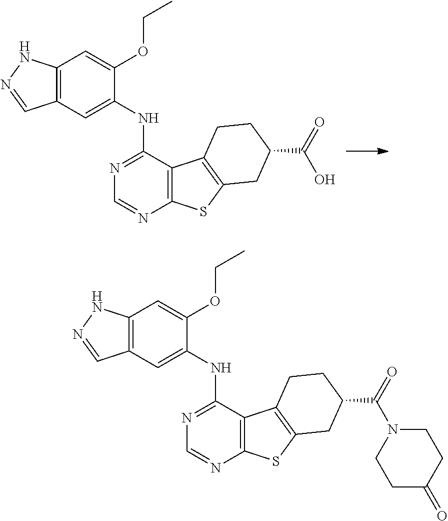 [SMILES: CCOc1cc2[nH]ncc2cc1Nc1ncnc2sc3c(c12)CC[C@H](C(=O)O)C3 ; CCOc1cc2[nH]ncc2cc1Nc1ncnc2sc3c(c12)CC[C@H](C(=O)N1CCC(=O)CC1)C3]

100 mg (244 μmol) (7S)-4-[(6-ethoxy-1H-indazol-5-yl)amino]-5,6,7,8-tetrahydro[1]benzothieno[2,3-d]pyrimidine-7-carboxylic acid (prepared according to intermediate example 65a) were transformed in analogy to example 1 using piperidin-4-one to give after working up and purification 66.8 mg (53%) of the title compound.

¹H-NMR (DMSO-d6): δ=1.46 (3H), 1.90 (1H), 2.12 (1H), 2.35-2.56 (3H), 2.97 (2H), 3.18-3.41 (4H), 3.68-3.97 (4H), 4.20 (2H), 7.04 (1H), 7.99 (1H), 8.35 (1H), 8.52 (1H), 9.02 (1H), 12.82 (1H) ppm.

### Example 69

### Reference Example

### 1-({(7R)-4-[(6-Ethoxy-1H-indazol-5-yl)amino]-5,6,7,8-tetrahydro[1]benzothieno[2,3-d]pyrimidin-7-yl}carbonyl)piperidin-4-one

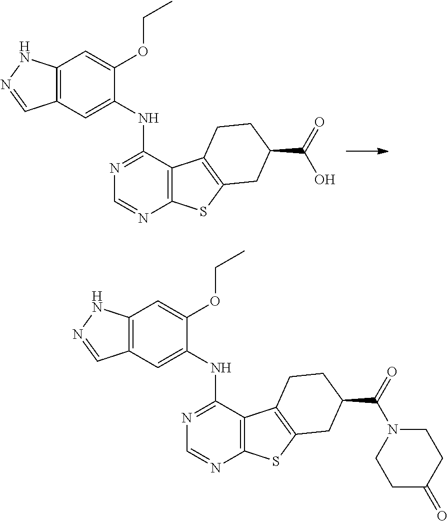 [SMILES: CCOc1cc2[nH]ncc2cc1Nc1ncnc2sc3c(c12)CC[C@@H](C(=O)O)C3 ; CCOc1cc2[nH]ncc2cc1Nc1ncnc2sc3c(c12)CC[C@@H](C(=O)N1CCC(=O)CC1)C3]

100 mg (244 μmol) (7R)-4-[(6-ethoxy-1H-indazol-5-yl)amino]-5,6,7,8-tetrahydro[1]benzothieno[2,3-d]pyrimidine-7-carboxylic acid (prepared according to intermediate example 69a) were transformed in analogy to example 1 using piperidin-4-one to give after working up and purification 3.7 mg (2%) of the title compound.

¹H-NMR (DMSO-d6): δ=1.46 (3H), 1.90 (1H), 2.12 (1H), 2.35-2.56 (3H), 2.97 (2H), 3.18-3.41 (4H), 3.68-3.97 (4H), 4.20 (2H), 7.04 (1H), 7.99 (1H), 8.35 (1H), 8.52 (1H), 9.02 (1H), 12.82 (1H) ppm.

### Example 69a

### (7R)-4-[(6-Ethoxy-1H-indazol-5-yl)amino]-5,6,7,8-tetrahydro[1]benzothieno[2,3-d]pyrimidine-7-carboxylic acid

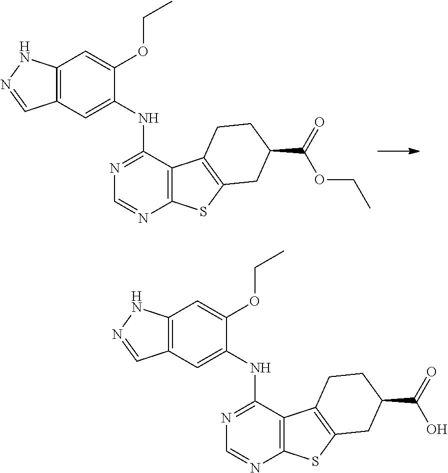 [SMILES: CCOC(=O)[C@@H]1CCc2c(sc3ncnc(Nc4cc5cn[nH]c5cc4OCC)c23)C1 ; CCOc1cc2[nH]ncc2cc1Nc1ncnc2sc3c(c12)CC[C@@H](C(=O)O)C3]

331 mg (757 μmol) ethyl (7R)-4-[(6-ethoxy-1H-indazol-5-yl)amino]-5,6,7,8-tetrahydro[1]benzothieno[2,3-d]pyrimidine-7-carboxylate (prepared according to intermediate example 69b) were transformed in analogy to intermediate example 1a to give after working up and purification 260 mg (80%) of the title compound.

### Example 69b

### Ethyl (7R)-4-[(6-ethoxy-1H-indazol-5-yl)amino]-5,6,7,8-tetrahydro[1]benzothieno[2,3-d]pyrimidine-7-carboxylate

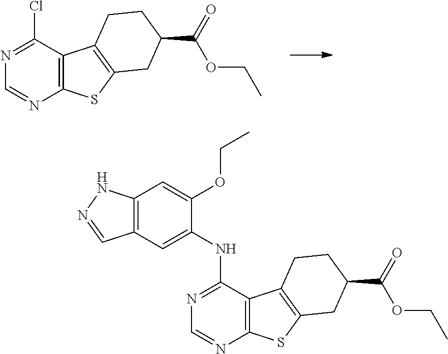

300 mg (1.01 mmol) ethyl (7R)-4-chloro-5,6,7,8-tetrahydro[1]benzothieno[2,3-d]pyrimidine-7-carboxylate (prepared according to intermediate example 2c) were transformed in analogy to intermediate example 1b using 6-ethoxy-1H-indazol-5-amine to give after working up and purification 336 mg (68%) of the title compound.

### Example 70

{(7S)-4-[(6-Ethoxy-1H-indazol-5-yl)amino]-5,6,7,8-tetrahydro[1]benzothieno[2,3-d]pyrimidin-7-yl}[(1S,4S)-2-oxa-5-azabicyclo[2.2.1]hept-5-yl]methanone

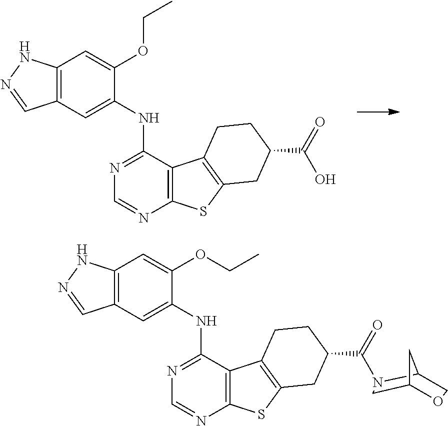

200 mg (488 μmol) (7S)-4-[(6-ethoxy-1H-indazol-5-yl)amino]-5,6,7,8-tetrahydro[1]benzothieno[2,3-d]pyrimidine-7-carboxylic acid (prepared according to intermediate example 65a) were transformed in analogy to example 1 using (1S,4S)-2-oxa-5-azabicyclo[2.2.1]heptane to give after working up and purification 70.0 mg (28%) of the title compound.

¹H-NMR (DMSO-d6): δ=1.46 (3H), 1.74-1.97 (3H), 2.07 (1H), 2.79-3.38 (6H), 3.54 (1H), 3.62-3.78 (2H), 4.20 (2H), 4.61+4.66 (1H), 4.76+4.88 (1H), 7.05 (1H), 7.99 (1H), 8.33+8.36 (1H), 8.52 (1H), 9.01 (1H), 12.82 (1H) ppm.

### Example 71

### {(7S)-4-[(6-Ethoxy-1H-indazol-5-yl)amino]-5,6,7,8-tetrahydro[1]benzothieno[2,3-d]pyrimidin-7-yl}[(1R,4R)-2-oxa-5-azabicyclo[2.2.1]hept-5-yl]methanone

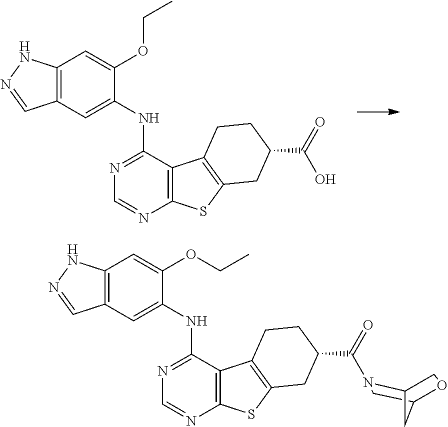

100 mg (244 μmol) (7S)-4-[(6-ethoxy-1H-indazol-5-yl)amino]-5,6,7,8-tetrahydro[1]benzothieno[2,3-d]pyrimidine-7-carboxylic acid (prepared according to intermediate example 65a) were transformed in analogy to example 1 using (1R,4R)-2-oxa-5-azabicyclo[2.2.1]heptane to give after working up and purification 55.9 mg (44%) of the title compound.

¹H-NMR (DMSO-d6): δ=1.48 (3H), 1.79 (1H), 1.83-1.95 (2H), 2.09 (1H), 2.79-3.28 (6H), 3.46-3.78 (3H), 4.21 (2H), 4.61+4.67 (1H), 4.77+4.85 (1H), 7.06 (1H), 7.99 (1H), 8.33+8.35 (1H), 8.52 (1H), 9.01 (1H), 12.80 (1H) ppm.

### Example 72

### {(7S)-4-[(6-Ethoxy-1H-indazol-5-yl)amino]-5,6,7,8-tetrahydro[1]benzothieno[2,3-d]pyrimidin-7-yl}(2-oxa-6-azaspiro[3.3]hept-6-yl)methanone

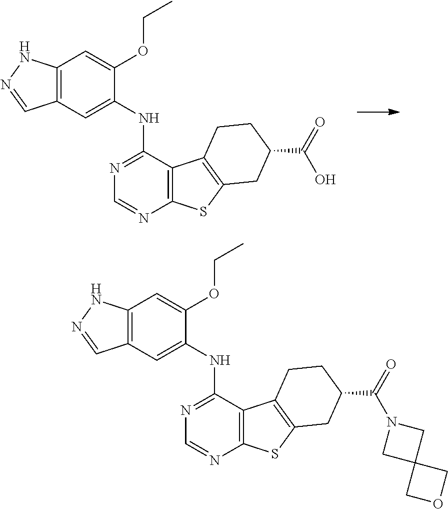 [SMILES: CCOc1cc2[nH]ncc2cc1Nc1ncnc2sc3c(c12)CC[C@H](C(=O)O)C3 ; CCOc1cc2[nH]ncc2cc1Nc1ncnc2sc3c(c12)CC[C@H](C(=O)N1CC2(COC2)C1)C3]

250 mg (611 μmol) (7S)-4-[(6-ethoxy-1H-indazol-5-yl)amino]-5,6,7,8-tetrahydro[1]benzothieno[2,3-d]pyrimidine-7-carboxylic acid (prepared according to intermediate example 65a) were transformed in analogy to example 1 using 2-oxa-6-azaspiro[3.3]heptane to give after working up and purification 170 mg (54%) of the title compound.

¹H-NMR (DMSO-d6): δ=1.47 (3H), 1.82 (1H), 2.05 (1H), 2.75 (1H), 2.80-2.96 (2H), 3.14 (1H), 3.27 (1H), 4.05 (2H), 4.20 (2H), 4.35-4.49 (2H), 4.68 (4H), 7.05 (1H), 7.99 (1H), 8.33 (1H), 8.51 (1H), 9.00 (1H), 12.81 (1H) ppm.

### Example 73

### (7S)-4-[(6-Ethoxy-1H-indazol-5-yl)amino]-N-(2-hydroxyethyl)-N-methyl-5,6,7,8-tetrahydro[1]benzothieno[2,3-d]pyrimidine-7-carboxamide

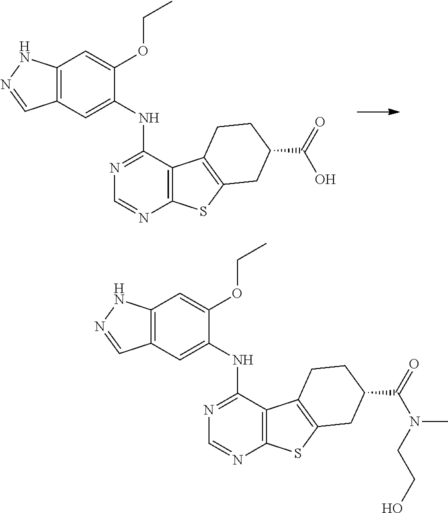 [SMILES: CCOc1cc2[nH]ncc2cc1Nc1ncnc2sc3c(c12)CC[C@H](C(=O)O)C3 ; CCOc1cc2[nH]ncc2cc1Nc1ncnc2sc3c(c12)CC[C@H](C(=O)N(C)CCO)C3]

100 mg (244 μmol) (7S)-4-[(6-ethoxy-1H-indazol-5-yl)amino]-5,6,7,8-tetrahydro[1]benzothieno[2,3-d]pyrimidine-7-carboxylic acid (prepared according to intermediate example 65a) were transformed in analogy to example 1 using 2-(methylamino)ethanol to give after working up and purification 12.2 mg (10%) of the title compound.

¹H-NMR (DMSO-d6): δ=1.41-1.53 (3H), 1.86 (1H), 2.08 (1H), 2.87+3.13 (3H), 2.92 (2H), 3.09-3.58 (7H), 4.21 (2H), 4.65+4.82 (1H), 7.05 (1H), 7.99 (1H), 8.35 (1H), 8.52 (1H), 9.02 (1H), 12.80 (1H) ppm.

### Example 74

### (7S)-4-[(6-Ethoxy-1H-indazol-5-yl)amino]-N-(2-hydroxy-2-methylpropyl)-N-methyl-5,6,7,8-tetrahydro[1]benzothieno[2,3-d]pyrimidine-7-carboxamide

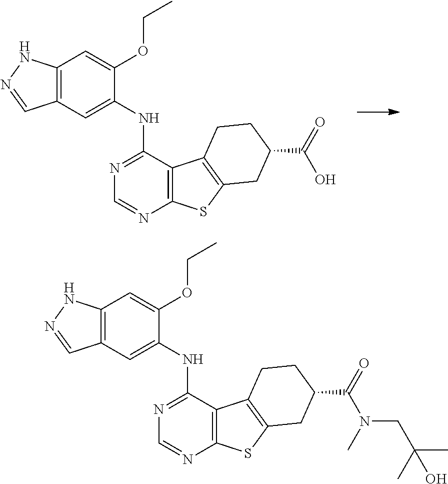

100 mg (244 μmol) (7S)-4-[(6-ethoxy-1H-indazol-5-yl)amino]-5,6,7,8-tetrahydro[1]benzothieno[2,3-d]pyrimidine-7-carboxylic acid (prepared according to intermediate example 65a) were transformed in analogy to example 1 using 2-methyl-1-(methylamino)propan-2-ol to give after working up and purification 20 mg (17%) of the title compound.

¹H-NMR (DMSO-d6): δ=1.01-1.16 (6H), 1.41-1.54 (3H), 1.87 (1H), 2.08 (1H), 2.86-3.02 (2H), 2.95+3.20 (3H), 3.15-3.44 (5H), 4.22 (2H), 4.51+4.56 (1H), 7.06 (1H), 7.99 (1H), 8.36 (1H), 8.52 (1H), 9.02+9.03 (1H), 12.82 (1H) ppm.

### Example 75

### [(3R,4R)-3,4-Dihydroxypyrrolidin-1-yl]{(7S)-4-[(6-ethoxy-1H-indazol-5-yl)amino]-5,6,7,8-tetrahydro[1]benzothieno[2,3-d]pyrimidin-7-yl}methanone

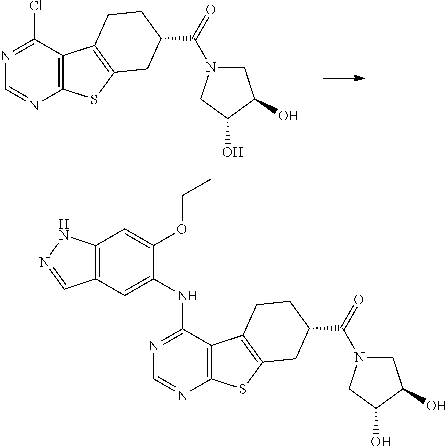

32 mg (90 μmol) [(7S)-4-chloro-5,6,7,8-tetrahydro[1]benzothieno[2,3-d]pyrimidin-7-yl][(3R,4R)-3,4-dihydroxypyrrolidin-1-yl]methanone (prepared according to intermediate example 75a) were transformed in analogy to intermediate example 1b using 6-ethoxy-1H-indazol-5-amine to give after working up and purification 21.0 mg (45%) of the title compound.

¹H-NMR (DMSO-d6): δ=1.46 (3H), 1.86 (1H), 2.07 (1H), 2.88-3.03 (3H), 3.17 (1H), 3.28 (2H), 3.40-3.51 (2H), 3.70 (1H), 3.93 (1H), 4.00 (1H), 4.20 (2H), 5.13 (1H), 5.17 (1H), 7.04 (1H), 7.98 (1H), 8.34 (1H), 8.51 (1H), 9.02 (1H), 12.80 (1H) ppm.

### Example 75a

### [(7S)-4-Chloro-5,6,7,8-tetrahydro[1]benzothieno[2,3-d]pyrimidin-7-yl][(3R,4R)-3,4-dihydroxypyrrolidin-1-yl]methanone

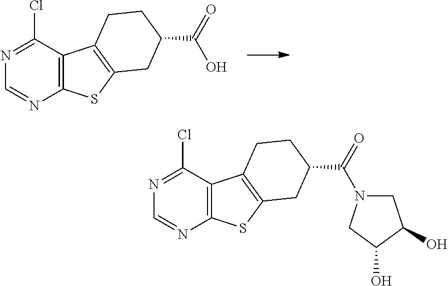 [SMILES: O=C(O)[C@H]1CCc2c(sc3ncnc(Cl)c23)C1 ; O=C([C@H]1CCc2c(sc3ncnc(Cl)c23)C1)N1C[C@@H](O)[C@H](O)C1]

150 mg (558 μmol) (7S)-4-chloro-5,6,7,8-tetrahydro[1]benzothieno[2,3-d]pyrimidine-7-carboxylic acid (prepared according to intermediate example 66b) were transformed in analogy to example 1 using (3R,4R)-pyrrolidine-3,4-diol to give after working up and purification 32.2 mg (15%) of the title compound.

### Example 76

### [(3S,4S)-3,4-Dihydroxypyrrolidin-1-yl]{(7S)-4-[(6-ethoxy-1H-indazol-5-yl)amino]-5,6,7,8-tetrahydro[1]benzothieno[2,3-d]pyrimidin-7-yl}methanone

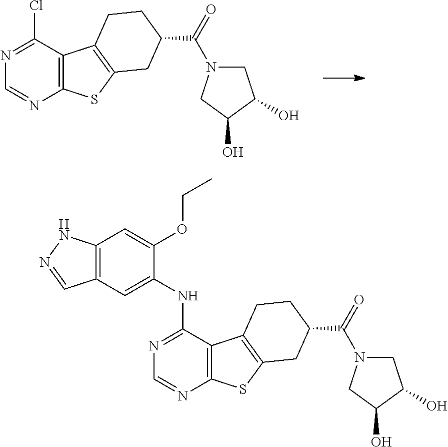

32 mg (90 μmol) [(7S)-4-chloro-5,6,7,8-tetrahydro[1]benzothieno[2,3-d]pyrimidin-7-yl][(3S,4S)-3,4-dihydroxypyrrolidin-1-yl]methanone (prepared according to intermediate example 76a) were transformed in analogy to intermediate example 1b using 6-ethoxy-1H-indazol-5-amine to give after working up and purification 26.4 mg (56%) of the title compound.

¹H-NMR (DMSO-d6): δ=1.46 (3H), 1.86 (1H), 2.09 (1H), 2.90-3.00 (3H), 3.18 (1H), 3.26-3.36 (2H), 3.38-3.46 (2H), 3.74 (1H), 3.93 (1H), 4.00 (1H), 4.20 (2H), 5.11 (1H), 5.21 (1H), 7.05 (1H), 7.99 (1H), 8.34 (1H), 8.52 (1H), 9.01 (1H), 12.80 (1H) ppm.

### Example 76a

### [(7S)-4-Chloro-5,6,7,8-tetrahydro[1]benzothieno[2,3-d]pyrimidin-7-yl][(3S,4S)-3,4-dihydroxypyrrolidin-1-yl]methanone

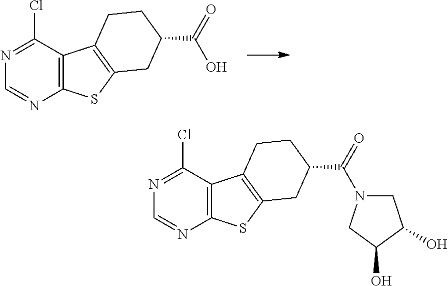 [SMILES: O=C(O)[C@H]1CCc2c(sc3ncnc(Cl)c23)C1 ; O=C([C@H]1CCc2c(sc3ncnc(Cl)c23)C1)N1C[C@H](O)[C@@H](O)C1]

150 mg (558 μmol) (7S)-4-chloro-5,6,7,8-tetrahydro[1]benzothieno[2,3-d]pyrimidine-7-carboxylic acid (prepared according to intermediate example 66b) were transformed in analogy to example 1 using (3S,4S)-pyrrolidine-3,4-diol to give after working up and purification 33.1 mg (15%) of the title compound.

### Example 77

### (7S)-4-[(6-Ethoxy-1H-indazol-5-yl)amino]-N,N-bis(2-hydroxyethyl)-5,6,7,8-tetrahydro[1]benzothieno[2,3-d]pyrimidine-7-carboxamide

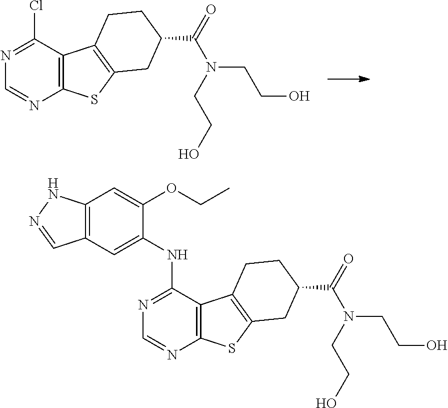

156 mg (438 μmol) (7S)-4-chloro-N,N-bis(2-hydroxyethyl)-5,6,7,8-tetrahydro[1]benzothieno[2,3-d]pyrimidine-7-carboxamide (prepared according to intermediate example 77a) were transformed in analogy to intermediate example 1b using 6-ethoxy-1H-indazol-5-amine to give after working up and purification 15.6 mg (7%) of the title compound.

¹H-NMR (DMSO-d6): δ=1.47 (3H), 1.87 (1H), 2.08 (1H), 2.92 (2H), 3.12-3.60 (11H), 4.21 (2H), 4.70 (1H), 4.84 (1H), 7.06 (1H), 7.99 (1H), 8.36 (1H), 8.52 (1H), 9.02 (1H), 12.82 (1H) ppm.

### Example 77a

### (7S)-4-chloro-N,N-bis(2-hydroxyethyl)-5,6,7,8-tetrahydro[1]benzothieno[2,3-d]pyrimidine-7-carboxamide

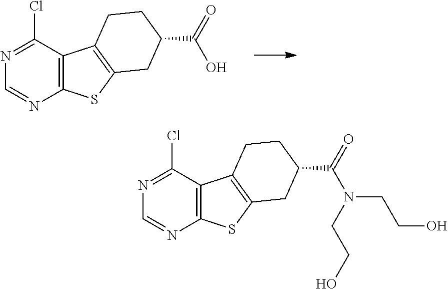 [SMILES: O=C(O)[C@H]1CCc2c(sc3ncnc(Cl)c23)C1 ; O=C([C@H]1CCc2c(sc3ncnc(Cl)c23)C1)N(CCO)CCO]

150 mg (558 μmol) (7S)-4-chloro-5,6,7,8-tetrahydro[1]benzothieno[2,3-d]pyrimidine-7-carboxylic acid (prepared according to intermediate example 66b) were transformed in analogy to example 1 using 2,2′-iminodiethanol to give after working up and purification 195 mg (98%) of the title compound.

### Example 78

### (7S)—N,N-Dimethyl-4-{[6-(propan-2-yloxy)-1H-indazol-5-yl]amino}-5,6,7,8-tetrahydro[1]benzothieno[2,3-d]pyrimidine-7-carboxamide

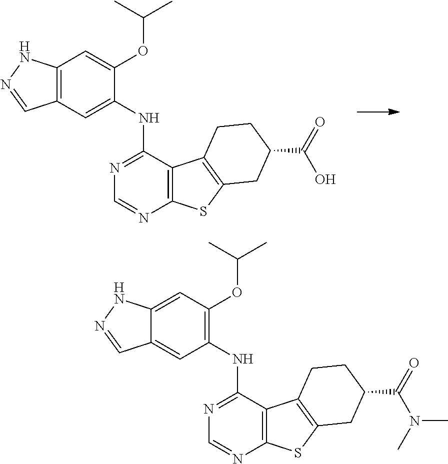

2.91 g (6.87 mmol) (7S)-4-[(6-isopropoxy-1H-indazol-5-yl)amino]-5,6,7,8-tetrahydro[1]benzothieno[2,3-d]pyrimidine-7-carboxylic acid (prepared according to intermediate example 78a) were transformed in analogy to example 1 to give after working up and purification 2.31 g (71%) of the title compound.

¹H-NMR (DMSO-d6): δ=1.40 (6H), 1.86 (1H), 2.08 (1H), 2.87 (3H), 2.93 (2H), 3.10 (3H), 3.16-3.29 (3H), 4.88 (1H), 7.11 (1H), 7.98 (1H), 8.36 (1H), 8.52 (1H), 9.06 (1H), 12.75 (1H) ppm.

### Example 78a

### (7S)-4-[(6-Isopropoxy-1H-indazol-5-yl)amino]-5,6,7,8-tetrahydro[1]benzothieno[2,3-d]pyrimidine-7-carboxylic acid

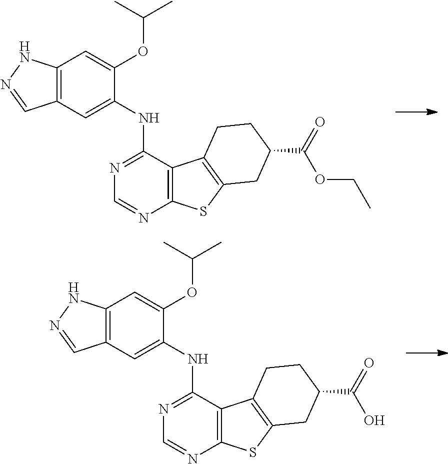

1.75 g (3.88 mmol) ethyl (7S)-4-[(6-isopropoxy-1H-indazol-5-yl)amino]-5,6,7,8-tetrahydro[1]benzothieno[2,3-d]pyrimidine-7-carboxylate (prepared according to intermediate example 78b) were transformed in analogy to intermediate example 1a to give after working up and purification 1.48 g (86%) of the title compound.

### Example 78b

### Ethyl (7S)-4-[(6-isopropoxy-1H-indazol-5-yl)amino]-5,6,7,8-tetrahydro[1]benzothieno[2,3-d]pyrimidine-7-carboxylate

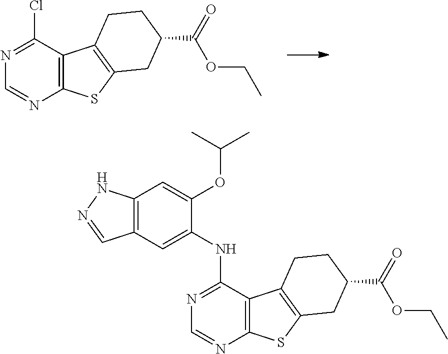

930 mg (3.13 mmol) ethyl (7S)-4-chloro-5,6,7,8-tetrahydro[1]benzothieno[2,3-d]pyrimidine-7-carboxylate (prepared according to intermediate example 1c) were transformed in analogy to intermediate example 1b using 6-isopropoxy-1H-indazol-5-amine to give after working up and purification 990 mg (63%) of the title compound.

### Example 79

### Reference Example

### (7R)—N,N-Dimethyl-4-{[6-(propan-2-yloxy)-1H-indazol-5-yl]amino}-5,6,7,8-tetrahydro[1]benzothieno[2,3-d]pyrimidine-7-carboxamide

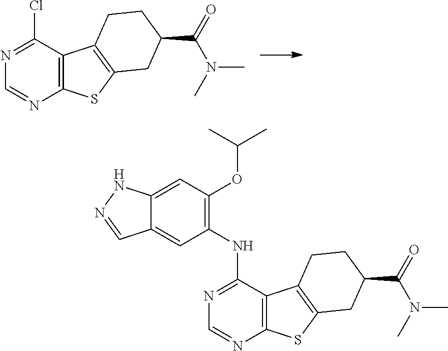

1.22 g (4.11 mmol) (7R)-4-chloro-N,N-dimethyl-5,6,7,8-tetrahydro[1]benzothieno[2,3-d]pyrimidine-7-carboxamide (prepared according to intermediate example 79a) were transformed in analogy to intermediate example 1b using 6-isopropoxy-1H-indazol-5-amine to give after working up and purification 985 mg (51%) of the title compound.

¹H-NMR (DMSO-d6): δ=1.40 (6H), 1.86 (1H), 2.08 (1H), 2.87 (3H), 2.93 (2H), 3.10 (3H), 3.16-3.29 (3H), 4.88 (1H), 7.11 (1H), 7.98 (1H), 8.36 (1H), 8.52 (1H), 9.06 (1H), 12.75 (1H) ppm.

### Example 79a

### (7R)-4-Chloro-N,N-dimethyl-5,6,7,8-tetrahydro[1]benzothieno[2,3-d]pyrimidine-7-carboxamide

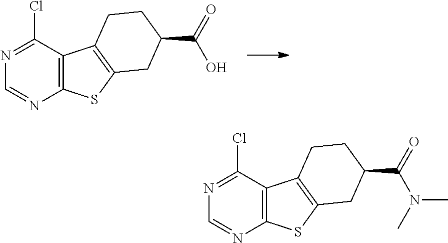 [SMILES: O=C(O)[C@@H]1CCc2c(sc3ncnc(Cl)c23)C1 ; CN(C)C(=O)[C@@H]1CCc2c(sc3ncnc(Cl)c23)C1]

3.00 g (11.16 mmol) (7R)-4-chloro-5,6,7,8-tetrahydro[1]benzothieno[2,3-d]pyrimidine-7-carboxylic acid (prepared according to intermediate example 79b) were transformed in analogy to example 1 to give after working up and purification 2.43 g (70%) of the title compound.

### Example 79b

### (7R)-4-Chloro-5,6,7,8-tetrahydro[1]benzothieno[2,3-d]pyrimidine-7-carboxylic acid

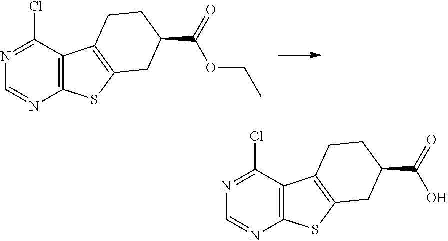

5.35 g (18.03 mmol) ethyl (7R)-4-chloro-5,6,7,8-tetrahydro[1]benzothieno[2,3-d]pyrimidine-7-carboxylate (prepared according to intermediate example 2c) were transformed in analogy to intermediate example 1a to give after working up and purification 4.69 g (92%) of the title compound.

### Example 80

### (7S)—N-Ethyl-N-(propan-2-yl)-4-{[6-(propan-2-yloxy)-1H-indazol-5-yl]amino}-5,6,7,8-tetrahydro[1]benzothieno[2,3-d]pyrimidine-7-carboxamide

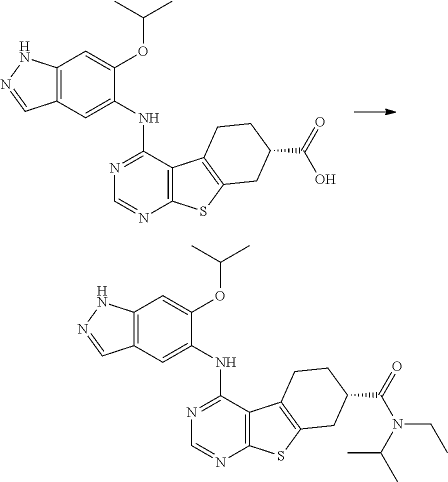

300 mg (708 μmol) (7S)-4-[(6-isopropoxy-1H-indazol-5-yl)amino]-5,6,7,8-tetrahydro[1]benzothieno[2,3-d]pyrimidine-7-carboxylic acid (prepared according to intermediate example 78a) were transformed in analogy to example 1 using N-ethylpropan-2-amine to give after working up and purification 106 mg (29%) of the title compound.

¹H-NMR (DMSO-d6): δ=1.02-1.26 (9H), 1.40 (6H), 1.92 (1H), 2.04 (1H), 2.85-3.38 (7H), 4.26+4.54 (1H), 4.89 (1H), 7.11 (1H), 8.00 (1H), 8.40 (1H), 8.54 (1H), 9.06 (1H), 12.61 (1H) ppm.

### Example 81

### (7S)—N-(2,2-Difluoroethyl)-N-methyl-4-{[6-(propan-2-yloxy)-1H-indazol-5-yl]amino}-5,6,7,8-tetrahydro[1]benzothieno[2,3-d]pyrimidine-7-carboxamide

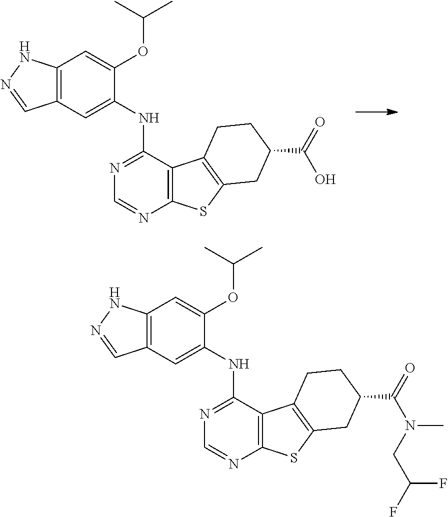

100 mg (236 μmol) (7S)-4-[(6-isopropoxy-1H-indazol-5-yl)amino]-5,6,7,8-tetrahydro[1]benzothieno[2,3-d]pyrimidine-7-carboxylic acid (prepared according to intermediate example 78a) were transformed in analogy to example 1 using 2,2-difluoro-N-methylethanamine to give after working up and purification 101 mg (81%) of the title compound.

¹H-NMR (DMSO-d6): δ=1.41 (6H), 1.90 (1H), 2.10 (1H), 2.87-3.04 (2H), 2.95+3.19 (3H), 3.16-3.29 (3H), 3.63-4.02 (2H), 4.89 (1H), 6.13+6.27 (1H), 7.11 (1H), 7.98 (1H), 8.36 (1H), 8.53 (1H), 9.07 (1H), 12.75 (1H) ppm.

### Example 82

### Reference Example

### (7R)—N-(2,2-Difluoroethyl)-N-methyl-4-{[6-(propan-2-yloxy)-1H-indazol-5-yl]amino}-5,6,7,8-tetrahydro[1]benzothieno[2,3-d]pyrimidine-7-carboxamide

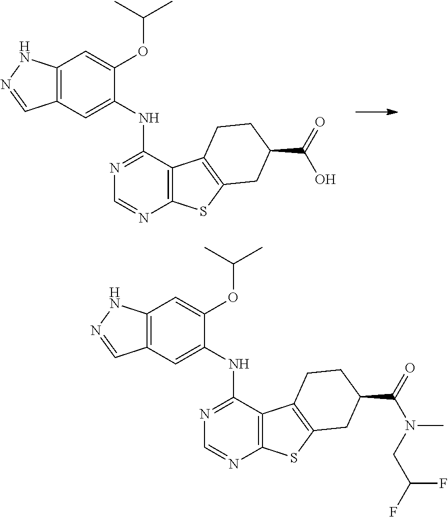

100 mg (236 μmol) (7R)-4-[(6-isopropoxy-1H-indazol-5-yl)amino]-5,6,7,8-tetrahydro[1]benzothieno[2,3-d]pyrimidine-7-carboxylic acid (prepared according to intermediate example 82a) were transformed in analogy to example 1 using 2,2-difluoro-N-methylethanamine to give after working up and purification 89.3 mg (72%) of the title compound.

¹H-NMR (DMSO-d6): δ=1.41 (6H), 1.90 (1H), 2.10 (1H), 2.87-3.04 (2H), 2.95+3.19 (3H), 3.16-3.29 (3H), 3.63-4.02 (2H), 4.89 (1H), 6.13+6.27 (1H), 7.11 (1H), 7.98 (1H), 8.36 (1H), 8.53 (1H), 9.07 (1H), 12.75 (1H) ppm.

### Example 82a

### (7R)-4-[(6-Isopropoxy-1H-indazol-5-yl)amino]-5,6,7,8-tetrahydro[1]benzothieno[2,3-d]pyrimidine-7-carboxylic acid

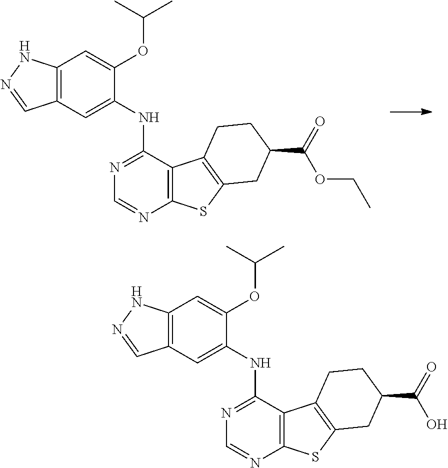

552 mg (1.22 mmol) ethyl (7R)-4-[(6-isopropoxy-1H-indazol-5-yl)amino]-5,6,7,8-tetrahydro[1]benzothieno[2,3-d]pyrimidine-7-carboxylate (prepared according to intermediate example 82b) were transformed in analogy to intermediate example 1a to give after working up and purification 461 mg (85%) of the title compound.

### Example 82b

### Ethyl (7R)-4-[(6-isopropoxy-1H-indazol-5-yl)amino]-5,6,7,8-tetrahydro[1]benzothieno[2,3-d]pyrimidine-7-carboxylate

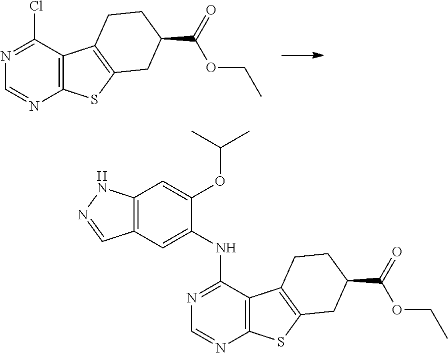

600 mg (2.02 mmol) ethyl (7R)-4-chloro-5,6,7,8-tetrahydro[1]benzothieno[2,3-d]pyrimidine-7-carboxylate (prepared according to intermediate example 2c) were transformed in analogy to intermediate example 1b using 6-isopropoxy-1H-indazol-5-amine (prepared according to intermediate example 82c) to give after working up and purification 556 mg (58%) of the title compound.

### Example 82c

### 6-Isopropoxy-1H-indazol-5-amine

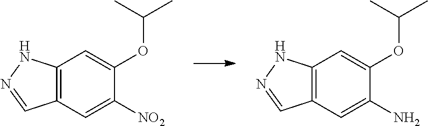 [SMILES: CC(C)Oc1cc2[nH]ncc2cc1[N+](=O)[O-] ; CC(C)Oc1cc2[nH]ncc2cc1N]

A mixture comprising 5.0 g (22.6 mmol) 6-isopropoxy-5-nitro-1H-indazole (purchased from Tractus chemicals, Unit 5, 3/F Harry Industrial Building; 4951 Au Pui Wan Street, Fo Tan; Shatin, New Territories; Hong Kong; Email: contact@tractuschem.com), 100 mL ethanol and 601 mg palladium on charcoal (10%) was heavily stirred under an atmosphere of hydrogen overnight. After filtration and removal of the solvent, the residue was washed with diethyl ether to give 3.64 g (80%) of the title compound.

### Example 83

### [(7S)-4-{[6-(Propan-2-yloxy)-1H-indazol-5-yl]amino}-5,6,7,8-tetrahydro[1]benzothieno[2,3-d]pyrimidin-7-yl](pyrrolidin-1-yl)methanone

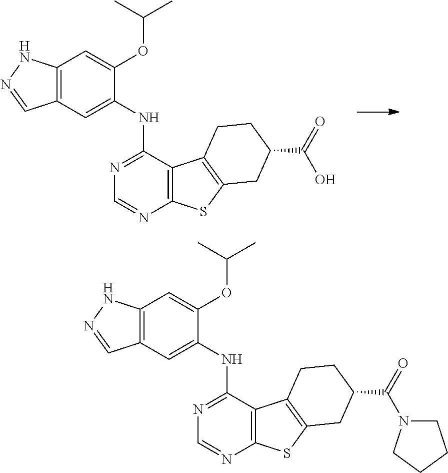

100 mg (236 μmol) (7S)-4-[(6-isopropoxy-1H-indazol-5-yl)amino]-5,6,7,8-tetrahydro[1]benzothieno[2,3-d]pyrimidine-7-carboxylic acid (prepared according to intermediate example 78a) were transformed in analogy to example 1 using pyrrolidine to give after working up and purification 102 mg (86%) of the title compound.

¹H-NMR (DMSO-d6): δ=1.40 (6H), 1.74-1.95 (5H), 2.11 (1H), 2.90-3.04 (3H), 3.21 (1H), 3.31-3.38 (3H), 3.48-3.62 (2H), 4.88 (1H), 7.11 (1H), 7.98 (1H), 8.36 (1H), 8.52 (1H), 9.06 (1H), 12.75 (1H) ppm.

### Example 84

### [(3S)-3-Methylmorpholin-4-yl][(7S)-4-{[6-(propan-2-yloxy)-1H-indazol-5-yl]amino}-5,6,7,8-tetrahydro[1]benzothieno[2,3-d]pyrimidin-7-yl]methanone

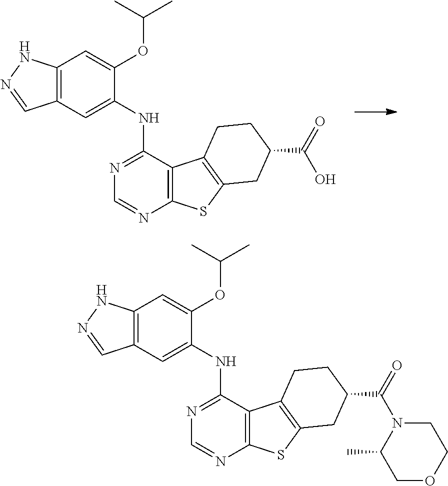

100 mg (236 μmol) (7S)-4-[(6-isopropoxy-1H-indazol-5-yl)amino]-5,6,7,8-tetrahydro[1]benzothieno[2,3-d]pyrimidine-7-carboxylic acid (prepared according to intermediate example 78a) were transformed in analogy to example 1 using (3S)-3-methylmorpholine to give after working up and purification 74.0 mg (62%) of the title compound.

¹H-NMR (DMSO-d6): δ=1.16+1.29 (3H), 1.40 (6H), 1.88 (1H), 2.05 (1H), 2.82-4.49 (12H), 4.88 (1H), 7.10 (1H), 7.98 (1H), 8.34 (1H), 8.52 (1H), 9.06 (1H), 12.77 (1H) ppm.

### Example 85

### [(3R)-3-Methylmorpholin-4-yl][(7S)-4-{[6-(propan-2-yloxy)-1H-indazol-5-yl]amino}-5,6,7,8-tetrahydro[1]benzothieno[2,3-d]pyrimidin-7-yl]methanone

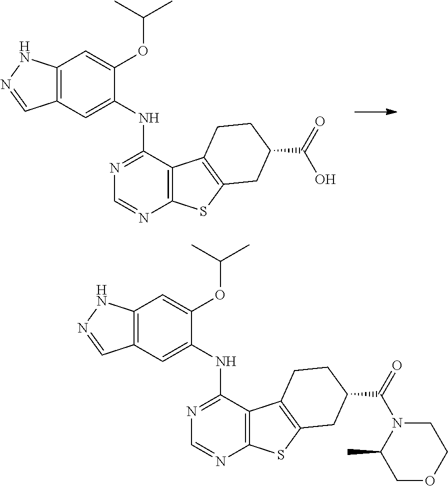

100 mg (236 μmol) (7S)-4-[(6-isopropoxy-1H-indazol-5-yl)amino]-5,6,7,8-tetrahydro[1]benzothieno[2,3-d]pyrimidine-7-carboxylic acid (prepared according to intermediate example 78a) were transformed in analogy to example 1 using (3R)-3-methylmorpholine to give after working up and purification 84.4 mg (71%) of the title compound.

¹H-NMR (DMSO-d6): δ=1.15+1.31 (3H), 1.40 (6H), 1.84-2.15 (2H), 2.79-4.47 (12H), 4.88 (1H), 7.10 (1H), 7.98 (1H), 8.36 (1H), 8.52 (1H), 9.07 (1H), 12.77 (1H) ppm.

### Example 86

### (1S,4S)-2-Oxa-5-azabicyclo[2.2.1]hept-5-yl[(7S)-4-{[6-(propan-2-yloxy)-1H-indazol-5-yl]amino}-5,6,7,8-tetrahydro[1]benzothieno[2,3-d]pyrimidin-7-yl]methanone

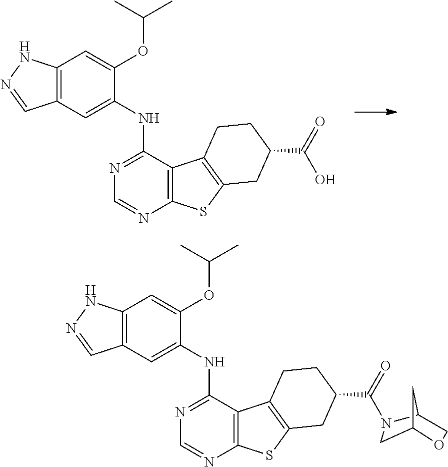

200 mg (472 μmol) (7S)-4-[(6-isopropoxy-1H-indazol-5-yl)amino]-5,6,7,8-tetrahydro[1]benzothieno[2,3-d]pyrimidine-7-carboxylic acid (prepared according to intermediate example 78a) were transformed in analogy to example 1 using (1S,4S)-2-oxa-5-azabicyclo[2.2.1]heptane to give after working up and purification 192 mg (76%) of the title compound.

¹H-NMR (DMSO-d6): δ=1.40 (6H), 1.75-1.97 (3H), 2.08 (1H), 2.83-3.05 (2H), 3.09-3.28 (3H), 3.52-3.78 (4H), 4.61+4.66 (1H), 4.76-4.92 (2H), 7.10 (1H), 7.98 (1H), 8.33+8.37 (1H), 8.52 (1H), 9.06 (1H), 12.75 (1H) ppm.

### Example 87

### (1R,4R)-2-Oxa-5-azabicyclo[2.2.1]hept-5-yl[(7S)-4-{[6-(propan-2-yloxy)-1H-indazol-5-yl]amino}-5,6,7,8-tetrahydro[1]benzothieno[2,3-d]pyrimidin-7-yl]methanone

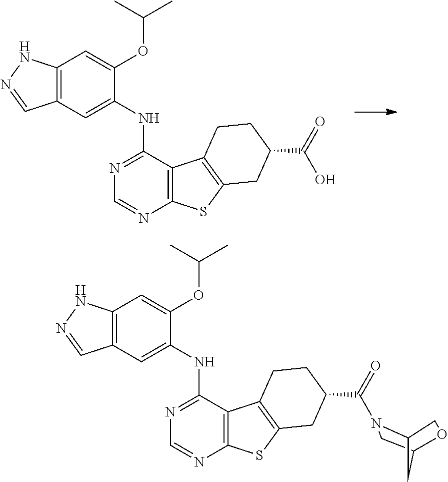

60 mg (142 μmol) (7S)-4-[(6-isopropoxy-1H-indazol-5-yl)amino]-5,6,7,8-tetrahydro[1]benzothieno[2,3-d]pyrimidine-7-carboxylic acid (prepared according to intermediate example 78a) were transformed in analogy to example 1 using (1R,4R)-2-oxa-5-azabicyclo[2.2.1]heptane to give after working up and purification 42.2 mg (56%) of the title compound.

¹H-NMR (DMSO-d6): δ=1.42 (6H), 1.75-1.96 (3H), 2.11 (1H), 2.81-3.36 (9H), 4.61+4.67 (1H), 4.77+4.86 (1H), 4.89 (1H), 7.11 (1H), 7.99 (1H), 8.35+8.37 (1H), 8.53 (1H), 9.07 (1H), 12.76 (1H) ppm.

### Example 88

### 2-Oxa-6-azaspiro[3.3]hept-6-yl[(7S)-4-{[6-(propan-2-yloxy)-1H-indazol-5-yl]amino}-5,6,7,8-tetrahydro[1]benzothieno[2,3-d]pyrimidin-7-yl]methanone

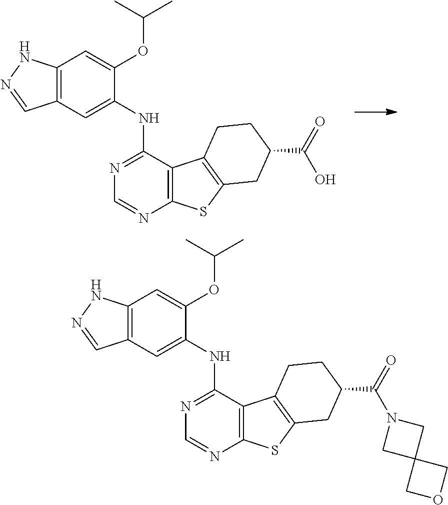

100 mg (236 μmol) (7S)-4-[(6-isopropoxy-1H-indazol-5-yl)amino]-5,6,7,8-tetrahydro[1]benzothieno[2,3-d]pyrimidine-7-carboxylic acid (prepared according to intermediate example 78a) were transformed in analogy to example 1 using 2-oxa-6-azaspiro[3.3]heptane to give after working up and purification 38.3 mg (31%) of the title compound.

¹H-NMR (DMSO-d6): δ=1.40 (6H), 1.84 (1H), 2.06 (1H), 2.70-2.97 (3H), 3.17 (1H), 3.28 (1H), 3.99-4.13 (2H), 4.41 (2H), 4.68 (4H), 4.88 (1H), 7.11 (1H), 7.99 (1H), 8.36 (1H), 8.52 (1H), 9.06 (1H), 12.77 (1H) ppm.

### Example 89

### Reference Example

### 2-Oxa-6-azaspiro[3.3]hept-6-yl[(7R)-4-{[6-(propan-2-yloxy)-1H-indazol-5-yl]amino}-5,6,7,8-tetrahydro[1]benzothieno[2,3-d]pyrimidin-7-yl]methanone

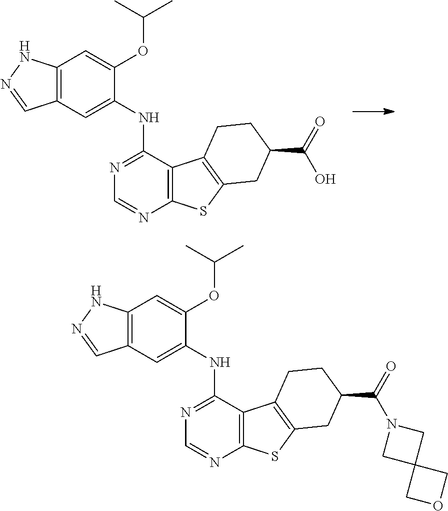

100 mg (236 μmol) (7R)-4-[(6-isopropoxy-1H-indazol-5-yl)amino]-5,6,7,8-tetrahydro[1]benzothieno[2,3-d]pyrimidine-7-carboxylic acid (prepared according to intermediate example 82a) were transformed in analogy to example 1 using 2-oxa-6-azaspiro[3.3]heptane to give after working up and purification 48.2 mg (38%) of the title compound.

¹H-NMR (DMSO-d6): δ=1.40 (6H), 1.84 (1H), 2.06 (1H), 2.70-2.97 (3H), 3.17 (1H), 3.28 (1H), 3.99-4.13 (2H), 4.41 (2H), 4.68 (4H), 4.88 (1H), 7.11 (1H), 7.99 (1H), 8.36 (1H), 8.52 (1H), 9.06 (1H), 12.77 (1H) ppm.

### Example 90

### (7S)—N-(2-Hydroxyethyl)-N-methyl-4-{[6-(propan-2-yloxy)-1H-indazol-5-yl]amino}-5,6,7,8-tetrahydro[1]benzothieno[2,3-d]pyrimidine-7-carboxamide

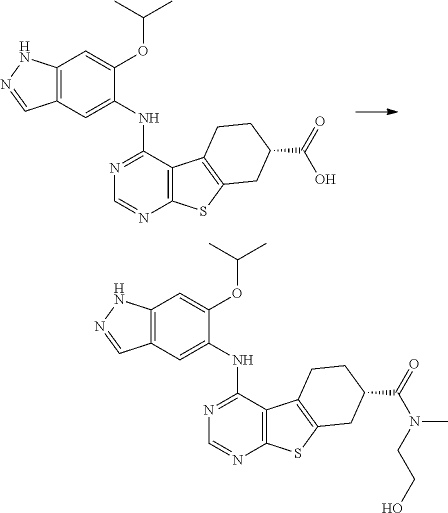

100 mg (236 μmol) (7S)-4-[(6-isopropoxy-1H-indazol-5-yl)amino]-5,6,7,8-tetrahydro[1]benzothieno[2,3-d]pyrimidine-7-carboxylic acid (prepared according to intermediate example 78a) were transformed in analogy to example 1 using 2-(methylamino)ethanol to give after working up and purification 66.1 mg (55%) of the title compound.

¹H-NMR (DMSO-d6): δ=1.40 (6H), 1.88 (1H), 2.10 (1H), 2.87+3.14 (3H), 2.94 (2H), 3.13-3.59 (7H), 4.65+4.82 (1H), 4.89 (1H), 7.11 (1H), 7.99 (1H), 8.37 (1H), 8.53 (1H), 9.07 (1H), 12.76 (1H) ppm.

### Example 91

### (7S)—N,N-bis(2-Hydroxyethyl)-4-{[6-(propan-2-yloxy)-1H-indazol-5-yl]amino}-5,6,7,8-tetrahydro[1]benzothieno[2,3-d]pyrimidine-7-carboxamide

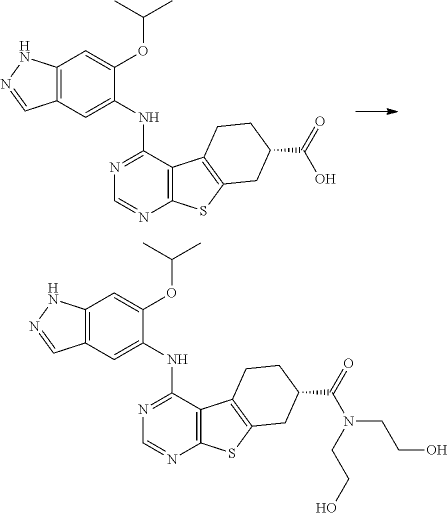

64 mg (151 μmol) (7S)-4-[(6-isopropoxy-1H-indazol-5-yl)amino]-5,6,7,8-tetrahydro[1]benzothieno[2,3-d]pyrimidine-7-carboxylic acid (prepared according to intermediate example 78a) were transformed in analogy to example 1 using 2,2′-iminodiethanol to give after working up and purification 40.0 mg (49%) of the title compound.

¹H-NMR (DMSO-d6): δ=1.41 (6H), 1.89 (1H), 2.10 (1H), 2.94 (2H), 3.13-3.60 (11H), 4.68 (1H), 4.82 (1H), 4.89 (1H), 7.11 (1H), 7.99 (1H), 8.37 (1H), 8.53 (1H), 9.07 (1H), 12.75 (1H) ppm.

### Example 92

### [(3S,4S)-3,4-Dihydroxypyrrolidin-1-yl][(7S)-4-{[6-(propan-2-yloxy)-1H-indazol-5-yl]amino}-5,6,7,8-tetrahydro[1]benzothieno[2,3-d]pyrimidin-7-yl]methanone

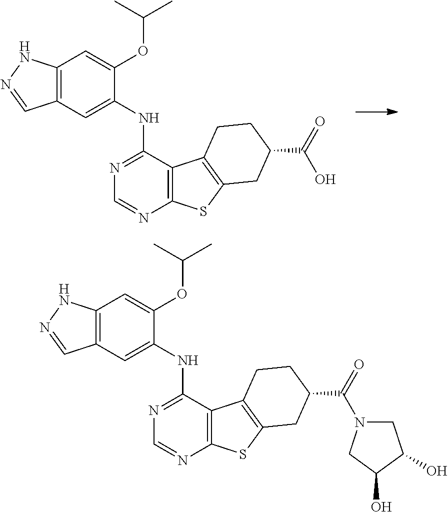

70 mg (165 μmol) (7S)-4-[(6-isopropoxy-1H-indazol-5-yl)amino]-5,6,7,8-tetrahydro[1]benzothieno[2,3-d]pyrimidine-7-carboxylic acid (prepared according to intermediate example 78a) were transformed in analogy to example 1 using (3S,4S)-pyrrolidine-3,4-diol to give after working up and purification 25.7 mg (29%) of the title compound.

¹H-NMR (DMSO-d6): δ=1.41 (6H), 1.88 (1H), 2.09 (1H), 2.87-3.07 (3H), 3.14-3.49 (5H), 3.74 (1H), 3.92 (1H), 3.99 (1H), 4.88 (1H), 5.14 (1H), 5.24 (1H), 7.11 (1H), 7.99 (1H), 8.37 (1H), 8.53 (1H), 9.07 (1H), 12.78 (1H) ppm.

### Example 93

### [(3R,4R)-3,4-Dihydroxypyrrolidin-1-yl][(7S)-4-{[6-(propan-2-yloxy)-1H-indazol-5-yl]amino}-5,6,7,8-tetrahydro[1]benzothieno[2,3-d]pyrimidin-7-yl]methanone

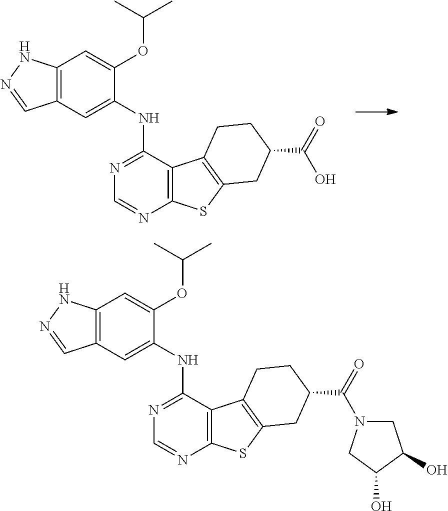

70 mg (165 μmol) (7S)-4-[(6-isopropoxy-1H-indazol-5-yl)amino]-5,6,7,8-tetrahydro[1]benzothieno[2,3-d]pyrimidine-7-carboxylic acid (prepared according to intermediate example 78a) were transformed in analogy to example 1 using (3R,4R)-pyrrolidine-3,4-diol to give after working up and purification 23.2 mg (26%) of the title compound.

¹H-NMR (DMSO-d6): δ=1.41 (6H), 1.89 (1H), 2.09 (1H), 2.88-3.05 (3H), 3.15-3.51 (5H), 3.70 (1H), 3.92 (1H), 4.00 (1H), 4.88 (1H), 5.12 (1H), 5.17 (1H), 7.11 (1H), 7.98 (1H), 8.37 (1H), 8.53 (1H), 9.07 (1H), 12.75 (1H) ppm.

### Example 94

### (7S)—N,N-Dimethyl-4-[(6-propoxy-1H-indazol-5-yl)amino]-5,6,7,8-tetrahydro[1]benzothieno[2,3-d]pyrimidine-7-carboxamide

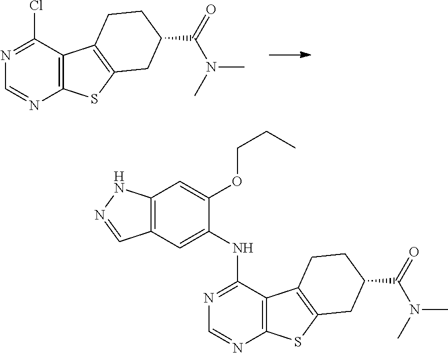 [SMILES: CN(C)C(=O)[C@H]1CCc2c(sc3ncnc(Cl)c23)C1 ; CCCOc1cc2[nH]ncc2cc1Nc1ncnc2sc3c(c12)CC[C@H](C(=O)N(C)C)C3]

81.2 mg (275 μmol) (7S)-4-chloro-N,N-dimethyl-5,6,7,8-tetrahydro[1]benzothieno[2,3-d]pyrimidine-7-carboxamide (prepared according to intermediate example 94a) were transformed in analogy to intermediate example 1b using 6-propoxy-1H-indazol-5-amine (prepared according to intermediate example 94b) to give after working up and purification 59.0 mg (44%) of the title compound.

¹H-NMR (DMSO-d6): δ=1.03 (3H), 1.75-1.92 (3H), 2.06 (1H), 2.87 (3H), 2.92 (3H), 3.09 (3H), 3.13-3.31 (2H), 4.10 (2H), 7.05 (1H), 7.99 (1H), 8.32 (1H), 8.51 (1H), 8.99 (1H), 12.82 (1H) ppm.

### Example 94a

### (7S)-4-Chloro-N,N-dimethyl-5,6,7,8-tetrahydro[1]benzothieno[2,3-d]pyrimidine-7-carboxamide

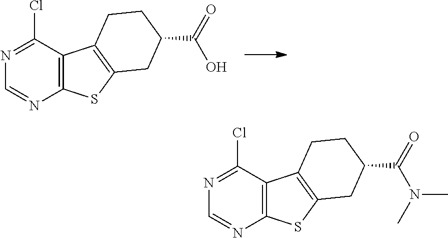 [SMILES: O=C(O)[C@H]1CCc2c(sc3ncnc(Cl)c23)C1 ; CN(C)C(=O)[C@H]1CCc2c(sc3ncnc(Cl)c23)C1]

372 mg (1.38 mmol) (7S)-4-chloro-5,6,7,8-tetrahydro[1]benzothieno[2,3-d]pyrimidine-7-carboxylic acid (prepared according to intermediate example 66b) were transformed in analogy to example 1 to give after working up and purification 308 mg (75%) of the title compound.

### Example 94b

### 6-Propoxy-1H-indazol-5-amine

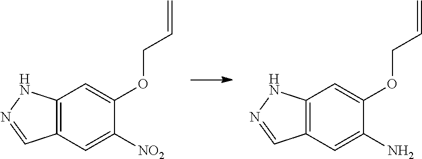 [SMILES: C=CCOc1cc2[nH]ncc2cc1[N+](=O)[O-] ; C=CCOc1cc2[nH]ncc2cc1N]

A mixture comprising 500 mg (2.28 mmol) 6-(allyloxy)-5-nitro-1H-indazole (prepared according to intermediate example 94c), 50 mL ethanol and 40 mg palladium on charcoal (10%) was shaken at 23° C. under an atmosphere of hydrogen for 5 hours. The catalyst and solvents were removed to give 430 mg (99%) of the title compound.

### Example 94c

### 6-(Allyloxy)-5-nitro-1H-indazole

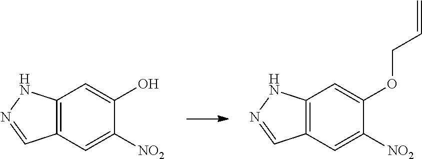 [SMILES: O=[N+]([O-])c1cc2cn[nH]c2cc1O ; C=CCOc1cc2[nH]ncc2cc1[N+](=O)[O-]]

To a mixture comprising 4.50 g (25.12 mmol) 5-nitro-1H-indazol-6-ol (prepared according to intermediate example 94d), 1.71 mL prop-2-en-1-ol, 7.91 g triphenylphosphane and 100 mL tetrahydrofurane were added at 3° C. 5.92 mL diisopropyl azodicarboxylate. The mixture was stirred at 23° C. overnight, concentrated and the residue was purified by chromatography to give 2.65 g (48%) of the title compound.

### Example 94d

### 5-Nitro-1H-indazol-6-ol

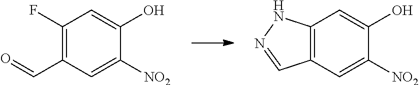 [SMILES: O=Cc1cc([N+](=O)[O-])c(O)cc1F ; O=[N+]([O-])c1cc2cn[nH]c2cc1O]

A mixture comprising 5.00 g (27.0 mmol) 2-fluoro-4-hydroxy-5-nitrobenzaldehyde (prepared according to intermediate example 94e), 100 mL ethanol and 6.57 mL hydrazine hydrate was heated at 100° C. for 2 hours. The mixture was acidified with hydrochloric acid, ethyl acetate was added, and the aqueous layer extracted with ethyl acetate. The combined organic layers were washed with hydrochloric acid, brine and dried over sodium sulfate. After filtration and removal of the solvent 2.33 g (48%) of the title compound were obtained.

### Example 94e

### 2-Fluoro-4-hydroxy-5-nitrobenzaldehyde

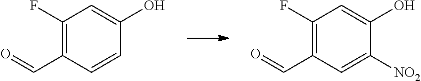 [SMILES: O=Cc1ccc(O)cc1F ; O=Cc1cc([N+](=O)[O-])c(O)cc1F]

A solution of 50.0 g (357 mmol) 2-fluoro-4-hydroxybenzaldehyde (CAS-No: 348-27-6) in 300 mL concentrated sulfuric acid was cooled to −15° C. A mixture comprising 22.5 mL nitric acid (65%) and 68.5 mL sulfuric acid was added slowly. After 1 hour the mixture was poured into ice-water. The precipitate was filtered, washed with water and hexane and dried to give 60.0 g (91%) of the title compound.

### Example 95

### (3-Hydroxy-3-methylazetidin-1-yl){(7S)-4-[(6-propoxy-1H-indazol-5-yl)amino]-5,6,7,8-tetrahydro[1]benzothieno[2,3-d]pyrimidin-7-yl}methanone

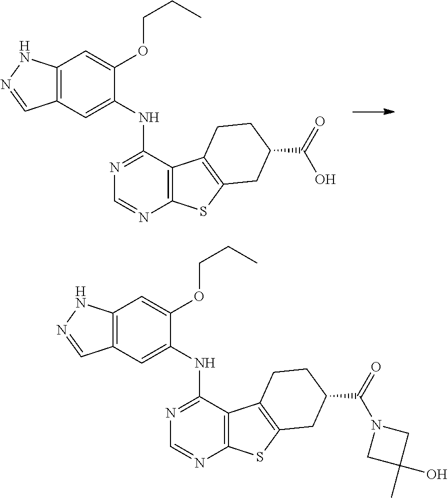

100 mg (236 μmol) (7S)-4-[(6-propoxy-1H-indazol-5-yl)amino]-5,6,7,8-tetrahydro[1]benzothieno[2,3-d]pyrimidine-7-carboxylic acid (prepared according to intermediate example 95a) were transformed in analogy to example 1 using 3-methylazetidin-3-ol to give after working up and purification 8.2 mg (7%) of the title compound.

¹H-NMR (DMSO-d6): δ=1.06 (3H), 1.39 (3H), 1.81-1.92 (3H), 2.06 (1H), 2.80 (1H), 2.84-2.97 (2H), 3.19 (2H), 3.69-3.78 (2H), 4.00-4.15 (4H), 7.08 (1H), 7.99 (1H), 8.31 (1H), 8.51 (1H), 8.99 (1H), 12.81 (1H) ppm.

### Example 95a

### (7S)-4-[(6-Propoxy-1H-indazol-5-yl)amino]-5,6,7,8-tetrahydro[1]benzothieno[2,3-d]pyrimidine-7-carboxylic acid

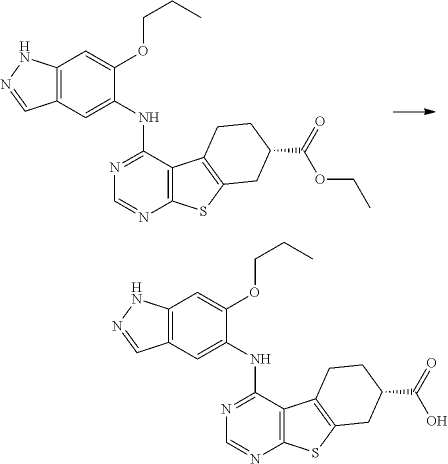 [SMILES: CCCOc1cc2[nH]ncc2cc1Nc1ncnc2sc3c(c12)CC[C@H](C(=O)OCC)C3 ; CCCOc1cc2[nH]ncc2cc1Nc1ncnc2sc3c(c12)CC[C@H](C(=O)O)C3]

475 mg (1.05 mmol) ethyl (7S)-4-[(6-propoxy-1H-indazol-5-yl)amino]-5,6,7,8-tetrahydro[1]benzothieno[2,3-d]pyrimidine-7-carboxylate (prepared according to intermediate example 95b) were transformed in analogy to intermediate example 1a to give after working up and purification 415 mg (93%) of the title compound.

### Example 95b

### Ethyl (7S)-4-[(6-propoxy-1H-indazol-5-yl)amino]-5,6,7,8-tetrahydro[1]benzothieno[2,3-d]pyrimidine-7-carboxylate

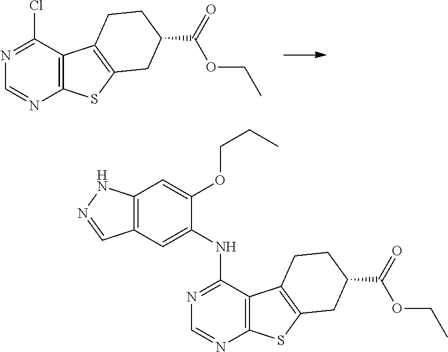

500 mg (1.69 mmol) ethyl (7S)-4-chloro-5,6,7,8-tetrahydro[1]benzothieno[2,3-d]pyrimidine-7-carboxylate (prepared according to intermediate example 1c) were transformed in analogy to intermediate example 1b using 6-propoxy-1H-indazol-5-amine (prepared according to intermediate example 94b) to give after working up and purification 480 mg (63%) of the title compound.

### Example 96

### (7S)-4-[(6-Hydroxy-1H-indazol-5-yl)amino]-N,N-dimethyl-5,6,7,8-tetrahydro[1]benzothieno[2,3-d]pyrimidine-7-carboxamide

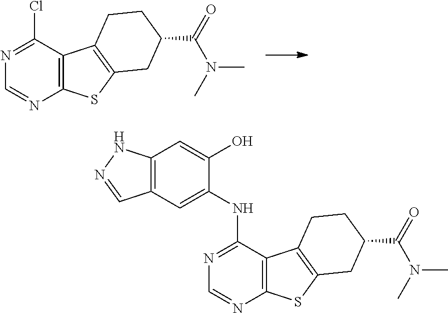 [SMILES: CN(C)C(=O)[C@H]1CCc2c(sc3ncnc(Cl)c23)C1 ; CN(C)C(=O)[C@H]1CCc2c(sc3ncnc(Nc4cc5cn[nH]c5cc4O)c23)C1]

145 mg (490 μmol) (7S)-4-chloro-N,N-dimethyl-5,6,7,8-tetrahydro[1]benzothieno[2,3-d]pyrimidine-7-carboxamide (prepared according to intermediate example 94a) were transformed in analogy to intermediate example 1b using 5-amino-1H-indazol-6-ol (prepared according to intermediate example 96a) to give after working up and purification 108 mg (54%) of the title compound.

¹H-NMR (DMSO-d6): δ=1.82 (1H), 2.09 (1H), 2.87 (3H), 2.90-2.97 (2H), 3.09 (3H), 3.14-3.28 (3H), 6.97 (1H), 7.92 (1H), 8.30 (1H), 8.48 (1H), 8.83 (1H), 10.76 (1H), 12.57 (1H) ppm.

### Example 96a

### 5-Amino-1H-indazol-6-ol

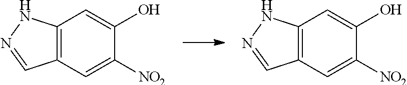 [SMILES: O=[N+]([O-])c1cc2cn[nH]c2cc1O ; O=[N+]([O-])c1cc2cn[nH]c2cc1O]

6.50 g (36.3 mmol) 5-nitro-1H-indazol-6-ol (prepared according to intermediate example 96b) were transformed in analogy to intermediate example 94b to give after working up and purification 5.28 g (98%) of the title compound.

### Example 96b

### 5-Nitro-1H-indazol-6-ol

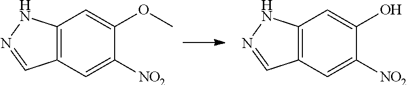

To a mixture comprising 5.00 g (25.89 mmol) 6-methoxy-5-nitro-1H-indazole and 240 mL dichloromethane were added 10.36 g aluminum trichloride at 23° C. The mixture was stirred at 55° C. overnight, cooled to 0° C. and water was added carefully. Methanol and dichloromethane were added, the precipitate filtered off and added to the organic layer. After removal of the solvent, the residue was purified by chromatography to give 3.11 g (67%) of the title compound.

### Example 97

### (7S)-4-[(6-methoxy-1H-indazol-5-yl)amino]-N-methyl-N-propyl-5,6,7,8-tetrahydro[1]benzothieno[2,3-d]pyrimidine-7-carboxamide

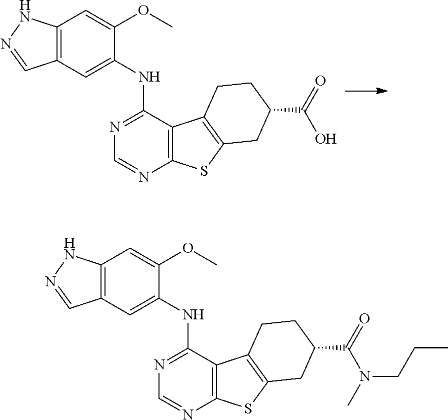 [SMILES: COc1cc2[nH]ncc2cc1Nc1ncnc2sc3c(c12)CC[C@H](C(=O)O)C3 ; CCCN(C)C(=O)[C@H]1CCc2c(sc3ncnc(Nc4cc5cn[nH]c5cc4OC)c23)C1]

250 mg (632 μmol) (7S)-4-[(6-methoxy-1H-indazol-5-yl)amino]-5,6,7,8-tetrahydro[1]benzothieno[2,3-d]pyrimidine-7-carboxylic acid (prepared according to intermediate example 20a) were transformed in analogy to example 1 using N-methylpropan-1-amine to give after working up and purification 212 mg (71%) of the title compound.

¹H-NMR (DMSO-d6): δ=0.83+0.87 (3H), 1.49+1.57 (2H), 1.84 (1H), 2.12 (1H), 2.85+3.08 (3H), 2.88-3.00 (2H), 3.10-3.44 (5H), 3.98 (3H), 7.09 (1H), 7.99 (1H), 8.21 (1H), 8.46 (1H), 8.76+8.79 (1H), 12.84 (1H) ppm.

### Example 98

### (7S)-4-{[6-(Dimethylamino)-1H-indazol-5-yl]amino}-N,N-dimethyl-5,6,7,8-tetrahydro[1]benzothieno[2,3-d]pyrimidine-7-carboxamide

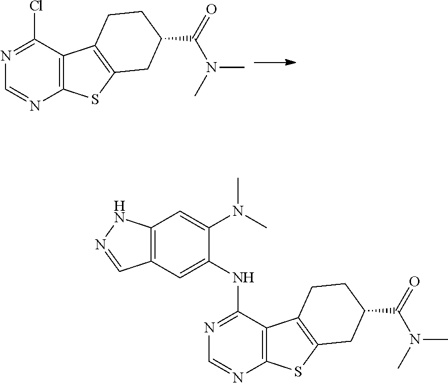 [SMILES: CN(C)C(=O)[C@H]1CCc2c(sc3ncnc(Cl)c23)C1 ; CN(C)C(=O)[C@H]1CCc2c(sc3ncnc(Nc4cc5cn[nH]c5cc4N(C)C)c23)C1]

50 mg (169 μmol) (7S)-4-chloro-N,N-dimethyl-5,6,7,8-tetrahydro[1]benzothieno[2,3-d]pyrimidine-7-carboxamide (prepared according to intermediate example 94a) were transformed in analogy to intermediate example 1b using N⁶,N⁶-dimethyl-1H-indazole-5,6-diamine (prepared according to intermediate example 98a) to give after working up and purification 14.6 mg (20%) of the title compound.

¹H-NMR (DMSO-d6): δ=1.84 (1H), 2.16 (1H), 2.72 (6H), 2.87 (3H), 2.93 (2H), 3.11 (3H), 3.16-3.27 (3H), 7.42 (1H), 8.03 (1H), 8.52 (1H), 8.99 (1H), 9.14 (1H), 12.90 (1H) ppm.

### Example 98a

### N⁶,N⁶-Dimethyl-1H-indazole-5,6-diamine

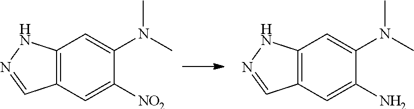

554 mg (2.69 mmol) N,N-dimethyl-5-nitro-1H-indazol-6-amine (prepared according to intermediate example 98b) were transformed in analogy to intermediate example 94b to give after working up and purification 173 mg (37%) of the title compound.

### Example 98b

### 5-Nitro-6-(trifluoromethoxy)-1H-indazole (A) and N,N-dimethyl-5-nitro-1H-indazol-6-amine (B)

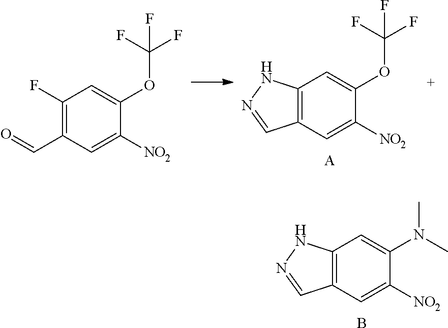

A mixture comprising 11.73 g (46.3 mmol) 2-fluoro-5-nitro-4-(trifluoromethoxy)benzaldehyde (prepared according to intermediate example 98c), 50 mL N,N-dimethylacetamide and 11.3 mL hydrazine hydrate was heated at 120° C. for 3 hours. The mixture was poured into water the precipitate was collected and purified by chromatography to give 3.44 g (30%) of the title compound A and 340 mg (3%) of the title compound B.

### Example 98c

### 2-Fluoro-5-nitro-4-(trifluoromethoxy)benzaldehyde

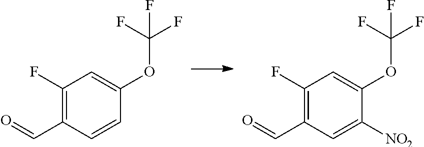 [SMILES: O=Cc1ccc(OC(F)(F)F)cc1F ; O=Cc1cc([N+](=O)[O-])c(OC(F)(F)F)cc1F]

28.20 g (135.5 mmol) 2-fluoro-4-(trifluoromethoxy)benzaldehyde (CAS-No: 1227628-83-2) were transformed in analogy to intermediate example 94e to give after working up and purification 34.87 g (100%) of the title compound.

### Example 99

### (7S)—N,N-Dimethyl-4-({6-[methyl(propan-2-yl)amino]-1H-indazol-5-yl}amino)-5,6,7,8-tetrahydro[1]benzothieno[2,3-d]pyrimidine-7-carboxamide

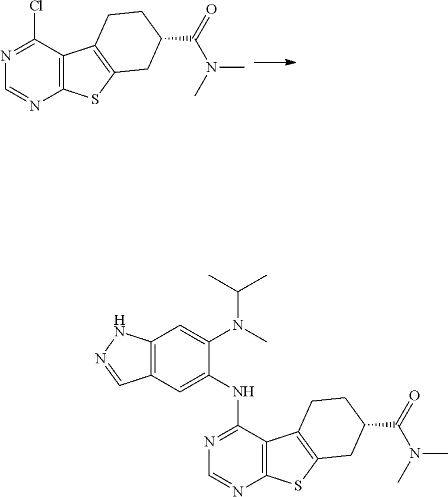 [SMILES: CN(C)C(=O)[C@H]1CCc2c(sc3ncnc(Cl)c23)C1 ; CC(C)N(C)c1cc2[nH]ncc2cc1Nc1ncnc2sc3c(c12)CC[C@H](C(=O)N(C)C)C3]

100 mg (338 μmol) (7S)-4-chloro-N,N-dimethyl-5,6,7,8-tetrahydro[1]benzothieno[2,3-d]pyrimidine-7-carboxamide (prepared according to intermediate example 94a) were transformed in analogy to intermediate example 1b using N⁶-isopropyl-N⁶-methyl-1H-indazole-5,6-diamine (prepared according to intermediate example 99a) to give after working up and purification 24.4 mg (16%) of the title compound.

¹H-NMR (DMSO-d6): δ=1.06 (6H), 1.88 (1H), 2.12 (1H), 2.64 (3H), 2.87 (3H), 2.94 (2H), 3.10 (3H), 3.18-3.36 (4H), 7.50 (1H), 8.05 (1H), 8.53 (1H), 9.10 (1H), 9.51 (1H), 12.87 (1H) ppm.

### Example 99a

### N⁶-Isopropyl-N⁶-methyl-1H-indazole-5,6-diamine

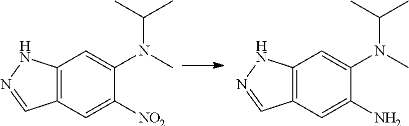 [SMILES: CC(C)N(C)c1cc2[nH]ncc2cc1[N+](=O)[O-] ; CC(C)N(C)c1cc2[nH]ncc2cc1N]

130 mg (555 μmol) N-isopropyl-N-methyl-5-nitro-1H-indazol-6-amine (prepared according to intermediate example 99b) were transformed in analogy to intermediate example 94b to give after working up and purification 69 mg (61%) of the title compound.

### Example 99b

### N-Isopropyl-N-methyl-5-nitro-1H-indazol-6-amine

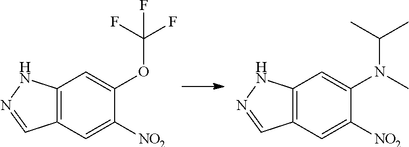 [SMILES: O=[N+]([O-])c1cc2cn[nH]c2cc1OC(F)(F)F ; CC(C)N(C)c1cc2[nH]ncc2cc1[N+](=O)[O-]]

A mixture comprising 1.30 g (5.26 mmol) 5-nitro-6-(trifluoromethoxy)-1H-indazole (prepared according to intermediate example 98b), 5.8 mL dimethyl sulfoxide and 5.48 mL N-methylpropan-2-amine was heated at 100° C. for 4 days. The mixture was poured into water and extracted with ethyl acetate. The organic layer was washed with brine and dried over sodium sulfate. After filtration and removal of the solvent the residue was purified by chromatography to give 154 mg (12%) of the title compound.

### Example 100

### (7S)-4-({6-[(2-Hydroxy-2-methylpropyl)amino]-1H-indazol-5-yl}amino)-N,N-dimethyl-5,6,7,8-tetrahydro[1]benzothieno[2,3-d]pyrimidine-7-carboxamide

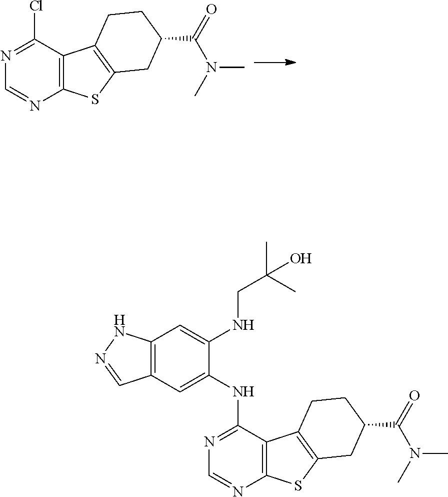 [SMILES: CN(C)C(=O)[C@H]1CCc2c(sc3ncnc(Cl)c23)C1 ; CN(C)C(=O)[C@H]1CCc2c(sc3ncnc(Nc4cc5cn[nH]c5cc4NCC(C)(C)O)c23)C1]

100 mg (338 μmol) (7S)-4-chloro-N,N-dimethyl-5,6,7,8-tetrahydro[1]benzothieno[2,3-d]pyrimidine-7-carboxamide (prepared according to intermediate example 94a) were transformed in analogy to intermediate example 1b using 1-[(5-amino-1H-indazol-6-yl)amino]-2-methylpropan-2-ol (prepared according to intermediate example 100a) to give after working up and purification 28 mg (17%) of the title compound.

¹H-NMR (DMSO-d6): δ=1.10 (6H), 1.77 (1H), 2.05 (1H), 2.87 (3H), 2.89-2.99 (4H), 3.09 (3H), 3.11-3.37 (3H), 4.46 (1H), 4.91 (1H), 6.55 (1H), 7.56 (1H), 7.79 (1H), 7.87 (1H), 8.18 (1H), 12.44 (1H) ppm.

### Example 100a

### 1-[(5-Amino-1H-indazol-6-yl)amino]-2-methylpropan-2-ol

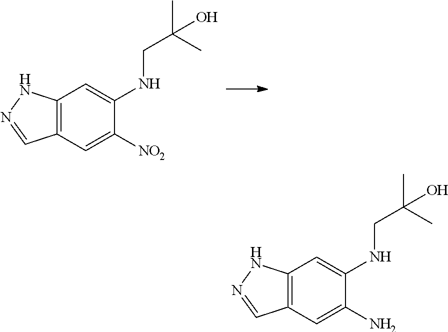 [SMILES: CC(C)(O)CNc1cc2[nH]ncc2cc1[N+](=O)[O-] ; CC(C)(O)CNc1cc2[nH]ncc2cc1N]

390 mg (1.56 mmol) 2-methyl-1-[(5-nitro-1H-indazol-6-yl)amino]propan-2-ol (prepared according to intermediate example 100b) were transformed in analogy to intermediate example 94b to give after working up and purification 126 mg (37%) of the title compound.

### Example 100b

### 2-Methyl-1-[(5-nitro-1H-indazol-6-yl)amino]propan-2-ol

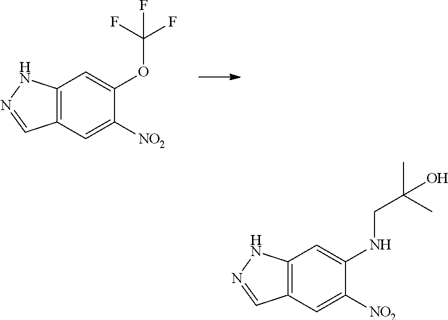 [SMILES: O=[N+]([O-])c1cc2cn[nH]c2cc1OC(F)(F)F ; CC(C)(O)CNc1cc2[nH]ncc2cc1[N+](=O)[O-]]

1.00 g (4.05 mmol) 5-nitro-6-(trifluoromethoxy)-1H-indazole (prepared according to intermediate example 98b) were transformed in analogy to intermediate example 99b using 1-amino-2-methylpropan-2-ol to give after working up and purification 394 mg (39%) of the title compound.

### Example 101

### (7S)-4-({6-[(2-Hydroxy-2-methylpropyl)(methyl)amino]-1H-indazol-5-yl}amino)-N,N-dimethyl-5,6,7,8-tetrahydro[1]benzothieno[2,3-d]pyrimidine-7-carboxamide

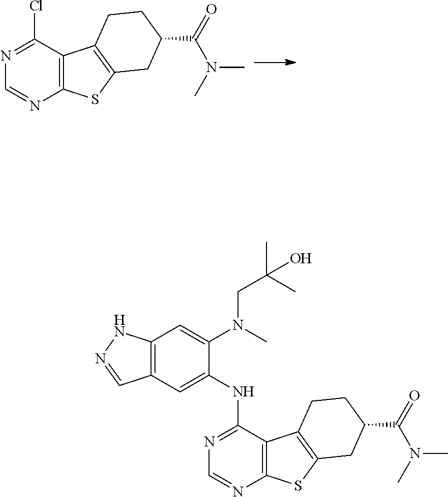 [SMILES: CN(C)C(=O)[C@H]1CCc2c(sc3ncnc(Cl)c23)C1 ; CN(C)C(=O)[C@H]1CCc2c(sc3ncnc(Nc4cc5cn[nH]c5cc4N(C)CC(C)(C)O)c23)C1]

100 mg (338 μmol) (7S)-4-chloro-N,N-dimethyl-5,6,7,8-tetrahydro[1]benzothieno[2,3-d]pyrimidine-7-carboxamide (prepared according to intermediate example 94a) were transformed in analogy to intermediate example 1b using 1-[(5-amino-1H-indazol-6-yl)(methyl)amino]-2-methylpropan-2-ol (prepared according to intermediate example 101a) to give after working up and purification 46 mg (28%) of the title compound.

¹H-NMR (DMSO-d6): δ=1.02 (6H), 1.81 (1H), 2.13 (1H), 2.73 (3H), 2.87 (3H), 2.90-2.97 (2H), 3.04-3.38 (4H), 3.09 (3H), 3.46 (1H), 4.33 (1H), 7.45 (1H), 8.02 (1H), 8.50 (1H), 9.00 (1H), 9.21 (1H), 12.83 (1H) ppm.

### Example 101a

### 1-[(5-Amino-1H-indazol-6-yl)(methyl)amino]-2-methylpropan-2-ol

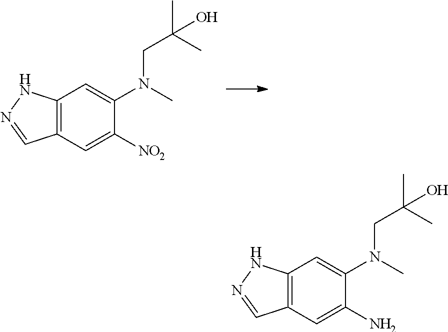 [SMILES: CN(CC(C)(C)O)c1cc2[nH]ncc2cc1[N+](=O)[O-] ; CN(CC(C)(C)O)c1cc2[nH]ncc2cc1N]

670 mg (2.54 mmol) 2-methyl-1-[methyl(5-nitro-1H-indazol-6-yl)amino]propan-2-ol (prepared according to intermediate example 101b) were transformed in analogy to intermediate example 94b to give after working up and purification 194 mg (33%) of the title compound.

### Example 101b

### 2-Methyl-1-[methyl(5-nitro-1H-indazol-6-yl)amino]propan-2-ol

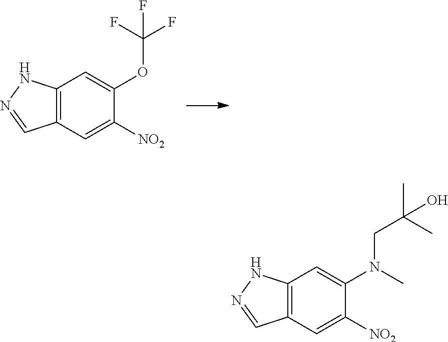 [SMILES: O=[N+]([O-])c1cc2cn[nH]c2cc1OC(F)(F)F ; CN(CC(C)(C)O)c1cc2[nH]ncc2cc1[N+](=O)[O-]]

1.00 g (4.05 mmol) 5-nitro-6-(trifluoromethoxy)-1H-indazole (prepared according to intermediate example 98b) were transformed in analogy to intermediate example 99b using 2-methyl-1-(methylamino)propan-2-ol to give after working up and purification 673 mg (63%) of the title compound.

### Example 102

### (7S)-4-{[6-(Azetidin-1-yl)-1H-indazol-5-yl]amino}-N,N-dimethyl-5,6,7,8-tetrahydro[1]benzothieno[2,3-d]pyrimidine-7-carboxamide

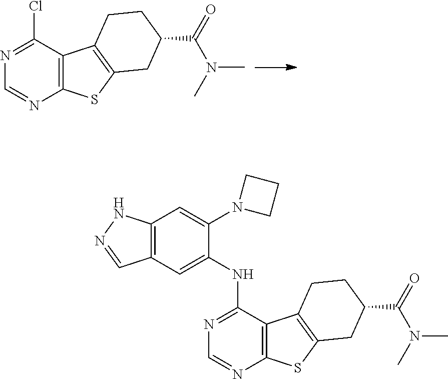 [SMILES: CN(C)C(=O)[C@H]1CCc2c(sc3ncnc(Cl)c23)C1 ; CN(C)C(=O)[C@H]1CCc2c(sc3ncnc(Nc4cc5cn[nH]c5cc4N4CCC4)c23)C1]

91.1 mg (308 μmol) (7S)-4-chloro-N,N-dimethyl-5,6,7,8-tetrahydro[1]benzothieno[2,3-d]pyrimidine-7-carboxamide (prepared according to intermediate example 94a) were transformed in analogy to intermediate example 1b using 6-(azetidin-1-yl)-1H-indazol-5-amine (prepared according to intermediate example 102a) to give after working up and purification 7 mg (5%) of the title compound.

¹H-NMR (DMSO-d6): δ=1.78 (1H), 2.05 (1H), 2.13 (2H), 2.82-2.94 (2H), 2.87 (3H), 3.09 (3H), 3.06-3.35 (3H), 3.75-3.85 (4H), 6.45 (1H), 7.50 (1H), 7.84 (1H), 7.90 (1H), 8.18 (1H), 12.56 (1H) ppm.

### Example 102a

### 6-(Azetidin-1-yl)-1H-indazol-5-amine

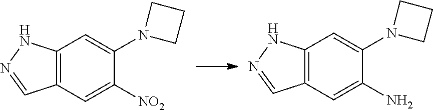 [SMILES: O=[N+]([O-])c1cc2cn[nH]c2cc1N1CCC1 ; Nc1cc2cn[nH]c2cc1N1CCC1]

570 mg (2.61 mmol) 6-(azetidin-1-yl)-5-nitro-1H-indazole (prepared according to intermediate example 102b) were transformed in analogy to intermediate example 94b to give after working up and purification 58 mg (12%) of the title compound.

### Example 102b

### 6-(Azetidin-1-yl)-5-nitro-1H-indazole

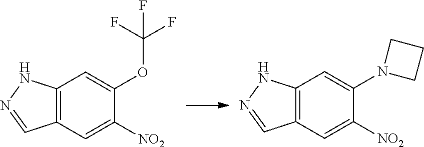 [SMILES: O=[N+]([O-])c1cc2cn[nH]c2cc1OC(F)(F)F ; O=[N+]([O-])c1cc2cn[nH]c2cc1N1CCC1]

1.00 g (4.05 mmol) 5-nitro-6-(trifluoromethoxy)-1H-indazole (prepared according to intermediate example 98b) were transformed in analogy to intermediate example 99b using aziridine to give after working up and purification 572 mg (65%) of the title compound.

### Example 103

### (7S)—N,N-Dimethyl-4-{[6-(pyrrolidin-1-yl)-1H-indazol-5-yl]amino}-5,6,7,8-tetrahydro[1]benzothieno[2,3-d]pyrimidine-7-carboxamide

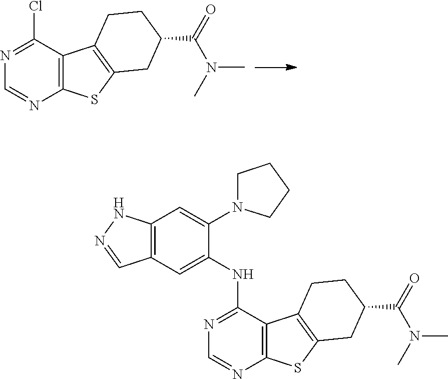 [SMILES: CN(C)C(=O)[C@H]1CCc2c(sc3ncnc(Cl)c23)C1 ; CN(C)C(=O)[C@H]1CCc2c(sc3ncnc(Nc4cc5cn[nH]c5cc4N4CCCC4)c23)C1]

100 mg (338 μmol) (7S)-4-chloro-N,N-dimethyl-5,6,7,8-tetrahydro[1]benzothieno[2,3-d]pyrimidine-7-carboxamide (prepared according to intermediate example 94a) were transformed in analogy to intermediate example 1b using 6-(pyrrolidin-1-yl)-1H-indazol-5-amine (prepared according to intermediate example 103a) to give after working up and purification 20 mg (13%) of the title compound.

¹H-NMR (DMSO-d6): δ=1.79-1.99 (5H), 2.07 (1H), 2.87 (3H), 2.93 (2H), 3.06-3.27 (7H), 3.09 (3H), 7.30 (1H), 7.98 (1H), 8.45 (1H), 8.73 (1H), 8.91 (1H), 12.78 (1H) ppm.

### Example 103a

### 6-(Pyrrolidin-1-yl)-1H-indazol-5-amine

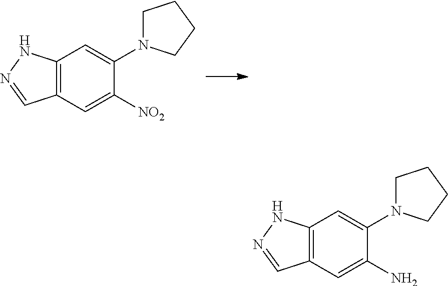 [SMILES: O=[N+]([O-])c1cc2cn[nH]c2cc1N1CCCC1 ; Nc1cc2cn[nH]c2cc1N1CCCC1]

1.03 g (4.45 mmol) 5-nitro-6-(pyrrolidin-1-yl)-1H-indazole (prepared according to intermediate example 103b) were transformed in analogy to intermediate example 94b to give after working up and purification 471 mg (52%) of the title compound.

### Example 103b

### 5-Nitro-6-(pyrrolidin-1-yl)-1H-indazole

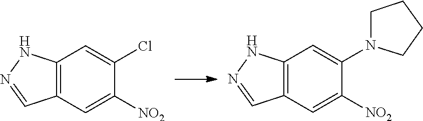 [SMILES: O=[N+]([O-])c1cc2cn[nH]c2cc1Cl ; O=[N+]([O-])c1cc2cn[nH]c2cc1N1CCCC1]

500 mg (2.53 mmol) 6-chloro-5-nitro-1H-indazole (CAS-No: 101420-98-8) were transformed in analogy to intermediate example 99b using pyrrolidine to give after working up and purification 567 mg (96%) of the title compound.

### Example 104

### (7S)-4-({6-[4-(Dimethylamino)piperidin-1-yl]-1H-indazol-5-yl}amino)-N,N-dimethyl-5,6,7,8-tetrahydro[1]benzothieno[2,3-d]pyrimidine-7-carboxamide

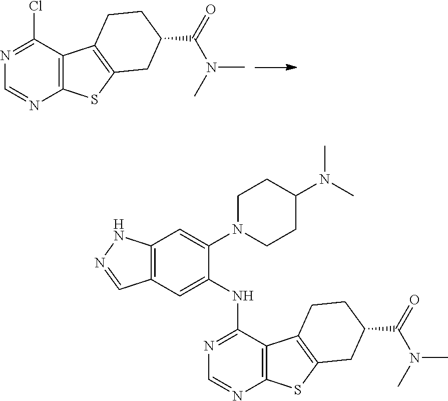 [SMILES: CN(C)C(=O)[C@H]1CCc2c(sc3ncnc(Cl)c23)C1 ; CN(C)C(=O)[C@H]1CCc2c(sc3ncnc(Nc4cc5cn[nH]c5cc4N4CCC(N(C)C)CC4)c23)C1]

75 mg (254 μmol) (7S)-4-chloro-N,N-dimethyl-5,6,7,8-tetrahydro[1]benzothieno[2,3-d]pyrimidine-7-carboxamide (prepared according to intermediate example 94a) were transformed in analogy to intermediate example 1b using 6-[4-(dimethylamino)piperidin-1-yl]-1H-indazol-5-amine (prepared according to intermediate example 104a) to give after working up and purification 20.1 mg (15%) of the title compound.

¹H-NMR (DMSO-d6): δ=1.60 (2H), 1.79-1.96 (3H), 2.13 (1H), 2.21 (1H), 2.24 (6H), 2.35 (1H), 2.72-3.10 (7H), 2.86 (3H), 3.07 (3H), 3.23 (1H), 7.47 (1H), 8.03 (1H), 8.52 (1H), 9.02 (1H), 9.07 (1H), 12.90 (1H) ppm.

### Example 104a

### 6-[4-(Dimethylamino)piperidin-1-yl]-1H-indazol-5-amine

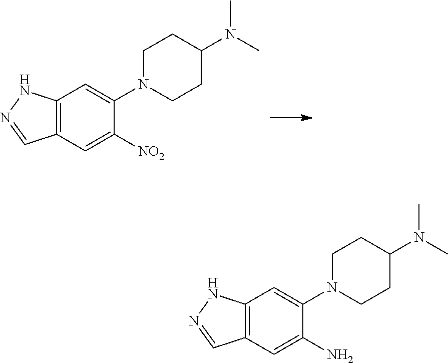 [SMILES: CN(C)C1CCN(c2cc3[nH]ncc3cc2[N+](=O)[O-])CC1 ; CN(C)C1CCN(c2cc3[nH]ncc3cc2N)CC1]

2.18 g (7.34 mmol) N,N-dimethyl-1-(5-nitro-1H-indazol-6-yl)piperidin-4-amine (prepared according to intermediate example 104b) were transformed in analogy to intermediate example 94b to give after working up and purification 1.48 g (75%) of the title compound.

### Example 104b

### N,N-Dimethyl-1-(5-nitro-1H-indazol-6-yl)piperidin-4-amine

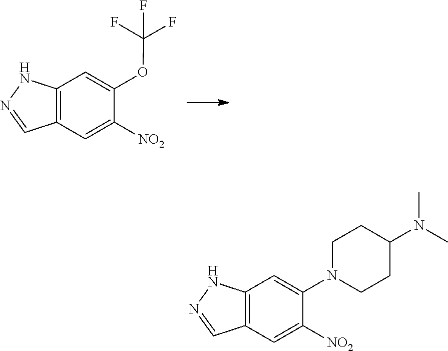 [SMILES: O=[N+]([O-])c1cc2cn[nH]c2cc1OC(F)(F)F ; CN(C)C1CCN(c2cc3[nH]ncc3cc2[N+](=O)[O-])CC1]

2.00 g (8.09 mmol) 5-nitro-6-(trifluoromethoxy)-1H-indazole (prepared according to intermediate example 98b) were transformed in analogy to intermediate example 99b using N,N-dimethylpiperidin-4-amine to give after working up and purification 2.19 g (94%) of the title compound.

### Example 105

### (7S)—N-Butyl-4-({6-[4-(dimethylamino)piperidin-1-yl]-1H-indazol-5-yl}amino)-N-methyl-5,6,7,8-tetrahydro[1]benzothieno[2,3-d]pyrimidine-7-carboxamide

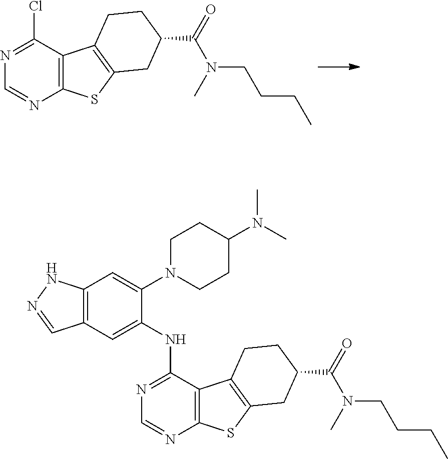 [SMILES: CCCCN(C)C(=O)[C@H]1CCc2c(sc3ncnc(Cl)c23)C1 ; CCCCN(C)C(=O)[C@H]1CCc2c(sc3ncnc(Nc4cc5cn[nH]c5cc4N4CCC(N(C)C)CC4)c23)C1]

64 mg (189 μmol) (7S)—N-butyl-4-chloro-N-methyl-5,6,7,8-tetrahydro[1]benzothieno[2,3-d]pyrimidine-7-carboxamide (prepared according to intermediate example 105a) were transformed in analogy to intermediate example 1b using 6-[4-(dimethylamino)piperidin-1-yl]-1H-indazol-5-amine (prepared according to intermediate example 104a) to give after working up and purification 14.0 mg (13%) of the title compound.

¹H-NMR (DMSO-d6): δ=0.86-0.93 (3H), 1.19-1.33 (2H), 1.41-1.68 (4H), 1.80-1.95 (3H), 2.11 (1H), 2.22 (6H), 2.26-2.38 (1H), 2.74-3.09 (6H), 2.84+3.04 (3H), 3.13-3.47 (5H), 7.47 (1H), 8.03 (1H), 8.52 (1H), 9.03+9.05 (1H), 9.08+9.13 (1H), 12.89 (1H) ppm.

### Example 105a

### (7S)—N-Butyl-4-chloro-N-methyl-5,6,7,8-tetrahydro[1]benzothieno[2,3-d]pyrimidine-7-carboxamide

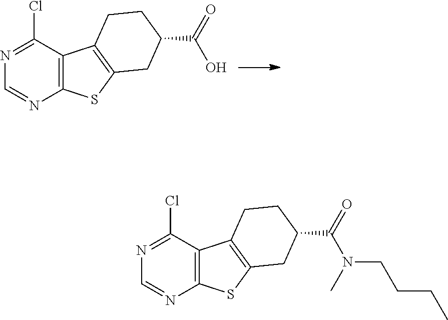 [SMILES: O=C(O)[C@H]1CCc2c(sc3ncnc(Cl)c23)C1 ; CCCCN(C)C(=O)[C@H]1CCc2c(sc3ncnc(Cl)c23)C1]

500 mg (1.86 mmol) (7S)-4-chloro-5,6,7,8-tetrahydro[1]benzothieno[2,3-d]pyrimidine-7-carboxylic acid (prepared according to intermediate example 66b) were transformed in analogy to example 1 using N-methylbutan-1-amine to give after working up and purification 283 mg (45%) of the title compound.

### Example 106

### (7S)—N,N-Dimethyl-4-{[6-(methylsulfanyl)-1H-indazol-5-yl]amino}-5,6,7,8-tetrahydro[1]benzothieno[2,3-d]pyrimidine-7-carboxamide

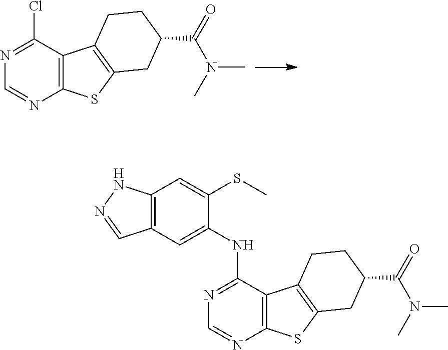 [SMILES: CN(C)C(=O)[C@H]1CCc2c(sc3ncnc(Cl)c23)C1 ; CSc1cc2[nH]ncc2cc1Nc1ncnc2sc3c(c12)CC[C@H](C(=O)N(C)C)C3]

125 mg (424 μmol) (7S)-4-chloro-N,N-dimethyl-5,6,7,8-tetrahydro[1]benzothieno[2,3-d]pyrimidine-7-carboxamide (prepared according to intermediate example 94a) were transformed in analogy to intermediate example 1b using 6-(methylsulfanyl)-1H-indazol-5-amine (prepared according to intermediate example 106a) to give after working up and purification 17 mg (9%) of the title compound.

¹H-NMR (DMSO-d6): δ=1.79 (1H), 2.07 (1H), 2.43 (3H), 2.87 (3H), 2.92 (2H), 3.09 (3H), 3.12-3.24 (2H), 3.32 (1H), 7.37 (1H), 7.89 (1H), 8.03 (1H), 8.13 (1H), 8.20 (1H), 12.98 (1H) ppm.

### Example 106a

### 6-(Methylsulfanyl)-1H-indazol-5-amine

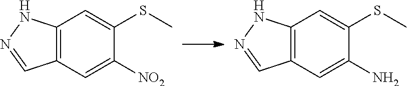

100 mg (478 μmol) 6-(methylsulfanyl)-5-nitro-1H-indazole (prepared according to intermediate example 106b) were transformed in analogy to intermediate example 94b to give after working up and purification 76 mg (89%) of the title compound.

### Example 106b

### 6-(Methylsulfanyl)-5-nitro-1H-indazole

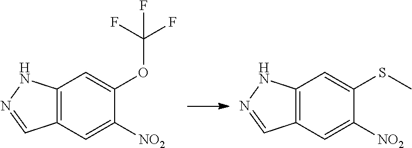 [SMILES: O=[N+]([O-])c1cc2cn[nH]c2cc1OC(F)(F)F ; CSc1cc2[nH]ncc2cc1[N+](=O)[O-]]

A mixture comprising 1.00 g (4.05 mmol) 5-nitro-6-(trifluoromethoxy)-1H-indazole (prepared according to intermediate example 98b), 20 mL N,N-dimethylformamide and 851 mg sodium methanethiolate was heated at 100° C. overnight. The mixture was poured into water, the precipitate collected, washed with water and dried. The crude product was purified by chromatography to give 368 mg (43%) of the title compound.

### Example 107

### (7S)-4-{[6-(2-Azidoethoxy)-1H-indazol-5-yl]amino}-N,N-dimethyl-5,6,7,8-tetrahydro[1]benzothieno[2,3-d]pyrimidine-7-carboxamide

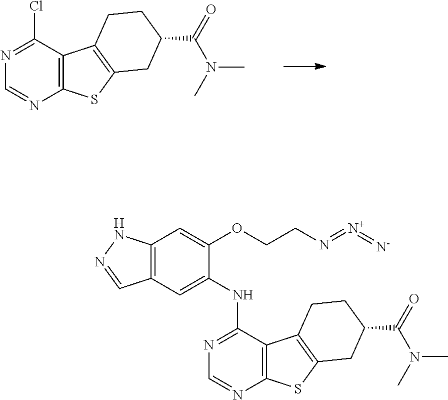 [SMILES: CN(C)C(=O)[C@H]1CCc2c(sc3ncnc(Cl)c23)C1 ; CN(C)C(=O)[C@H]1CCc2c(sc3ncnc(Nc4cc5cn[nH]c5cc4OCCN=[N+]=[N-])c23)C1]

100 mg (338 μmol) (7S)-4-chloro-N,N-dimethyl-5,6,7,8-tetrahydro[1]benzothieno[2,3-d]pyrimidine-7-carboxamide (prepared according to intermediate example 94a) were transformed in analogy to intermediate example 1b using 6-(2-azidoethoxy)-1H-indazol-5-amine (prepared according to intermediate example 107a) to give after working up and purification 105.3 mg (62%) of the title compound.

¹H-NMR (DMSO-d6): δ=1.84 (1H), 2.15 (1H), 2.83-3.01 (2H), 2.87 (3H), 3.10 (3H), 3.14-3.29 (3H), 3.85 (2H), 4.35 (2H), 7.12 (1H), 8.00 (1H), 8.21 (1H), 8.49 (1H), 8.93 (1H), 12.87 (1H) ppm.

### Example 107a

### 6-(2-Azidoethoxy)-1H-indazol-5-amine

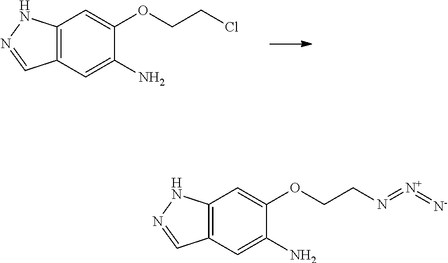 [SMILES: Nc1cc2cn[nH]c2cc1OCCCl ; [N-]=[N+]=NCCOc1cc2[nH]ncc2cc1N]

A mixture comprising 320 mg (1.51 mmol) 6-(2-chloroethoxy)-1H-indazol-5-amine (prepared according to intermediate example 107b), 4.8 mL N,N-dimethylformamide and 197 mg sodium azide was heated at 60° C. overnight. The mixture was filtered, the solvent removed and the residue was purified by chromatography to give 125 mg (38%) of the title compound.

### Example 107b

### 6-(2-Chloroethoxy)-1H-indazol-5-amine

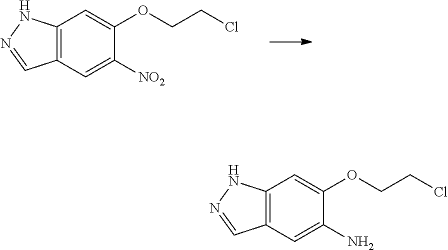 [SMILES: O=[N+]([O-])c1cc2cn[nH]c2cc1OCCCl ; Nc1cc2cn[nH]c2cc1OCCCl]

100 mg (414 μmol) 6-(2-chloroethoxy)-5-nitro-1H-indazole (prepared according to intermediate example 107c) were transformed in analogy to intermediate example 94b to give after working up and purification 78 mg (89%) of the title compound.

### Example 107c

### 6-(2-Chloroethoxy)-5-nitro-1H-indazole

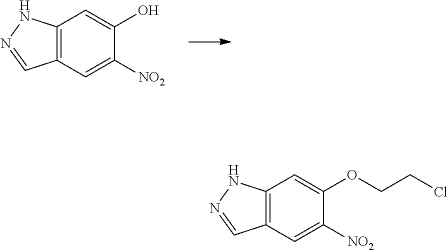 [SMILES: O=[N+]([O-])c1cc2cn[nH]c2cc1O ; O=[N+]([O-])c1cc2cn[nH]c2cc1OCCCl]

2.50 g (13.96 mmol) 5-nitro-1H-indazol-6-ol (prepared according to intermediate example 94d) were transformed in analogy to intermediate example 94c using 2-chloroethanol to give after working up and purification 820 mg (24%) of the title compound.

### Example 108

### (7S)-4-{[6-(2-Aminoethoxy)-1H-indazol-5-yl]amino}-N,N-dimethyl-5,6,7,8-tetrahydro[1]benzothieno[2,3-d]pyrimidine-7-carboxamide

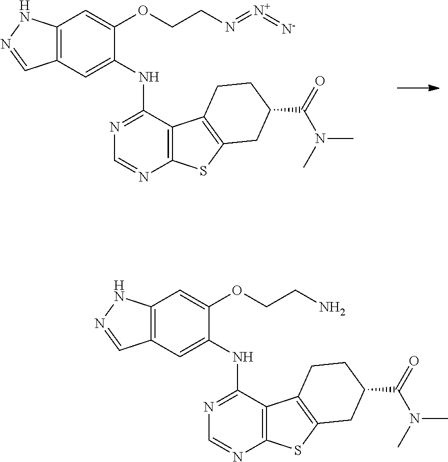 [SMILES: CN(C)C(=O)[C@H]1CCc2c(sc3ncnc(Nc4cc5cn[nH]c5cc4OCCN=[N+]=[N-])c23)C1 ; CN(C)C(=O)[C@H]1CCc2c(sc3ncnc(Nc4cc5cn[nH]c5cc4OCCN)c23)C1]

A mixture comprising 81.5 mg (171 μmol) (7S)-4-{[6-(2-Azidoethoxy)-1H-indazol-5-yl]amino}-N,N-dimethyl-5,6,7,8-tetrahydro[1]benzothieno[2,3-d]pyrimidine-7-carboxamide (prepared according to example 107), 7.5 mL N,N-dimethylformamide and 18 mg palladium on charcoal (10%) was vigorously stirred at 23° C. under an atmosphere of hydrogen overnight. The catalyst and solvent were removed and the residue crystallized from methanol to give 45.9 mg (55%) of the title compound.

¹H-NMR (DMSO-d6): δ=1.83 (1H), 2.12 (1H), 2.88 (3H), 2.93 (2H), 3.01 (2H), 3.10 (3H), 3.17 (1H), 3.21-3.39 (2H), 4.11 (2H), 7.06 (1H), 7.99 (1H), 8.38 (1H), 8.51 (1H), 9.00 (1H), 12.80 (1H) ppm.

### Example 109

### (7S)-4-({6-[2-(Dimethylamino)ethoxy]-1H-indazol-5-yl}amino)-N,N-dimethyl-5,6,7,8-tetrahydro[1]benzothieno[2,3-d]pyrimidine-7-carboxamide

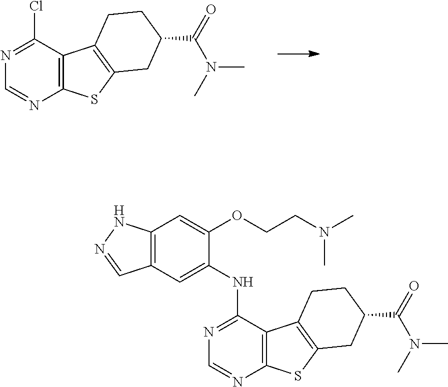 [SMILES: CN(C)C(=O)[C@H]1CCc2c(sc3ncnc(Cl)c23)C1 ; CN(C)CCOc1cc2[nH]ncc2cc1Nc1ncnc2sc3c(c12)CC[C@H](C(=O)N(C)C)C3]

40 mg (135 μmol) (7S)-4-chloro-N,N-dimethyl-5,6,7,8-tetrahydro[1]benzothieno[2,3-d]pyrimidine-7-carboxamide (prepared according to intermediate example 94a) were transformed in analogy to intermediate example 1b using 6-[2-(dimethylamino)ethoxy]-1H-indazol-5-amine (prepared according to intermediate example 109a) to give after working up and purification 30.5 mg (45%) of the title compound.

¹H-NMR (DMSO-d6): δ=1.85 (1H), 2.12 (1H), 2.21 (6H), 2.71 (2H), 2.87 (3H), 2.94 (2H), 3.10 (3H), 3.13-3.34 (3H), 4.25 (2H), 7.14 (1H), 7.99 (1H), 8.26 (1H), 8.50 (1H), 8.96 (1H), 12.83 (1H) ppm.

### Example 109a

### 6-[2-(Dimethylamino)ethoxy]-1H-indazol-5-amine

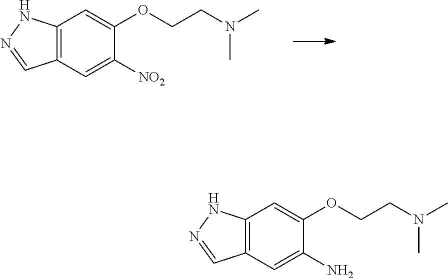 [SMILES: CN(C)CCOc1cc2[nH]ncc2cc1[N+](=O)[O-] ; CN(C)CCOc1cc2[nH]ncc2cc1N]

648 mg (2.59 mmol) N,N-dimethyl-2-[(5-nitro-1H-indazol-6-yl)oxy]ethanamine (prepared according to intermediate example 109b) were transformed in analogy to intermediate example 94b to give after working up and purification 312 mg (55%) of the title compound.

### Example 109b

### N,N-Dimethyl-2-[(5-nitro-1H-indazol-6-yl)oxy]ethanamine

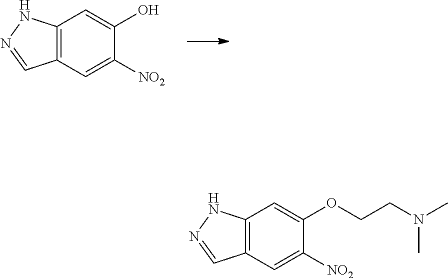 [SMILES: O=[N+]([O-])c1cc2cn[nH]c2cc1O ; CN(C)CCOc1cc2[nH]ncc2cc1[N+](=O)[O-]]

850 mg (4.74 mmol) 5-nitro-1H-indazol-6-ol (prepared according to intermediate example 94d) were transformed in analogy to intermediate example 94c using 2-(dimethylamino)ethanol to give after working up and purification 653 mg (55%) of the title compound.

### Example 110

### (7S)-4-{[6-(3-Azidopropoxy)-1H-indazol-5-yl]amino}-N,N-dimethyl-5,6,7,8-tetrahydro[1]benzothieno[2,3-d]pyrimidine-7-carboxamide

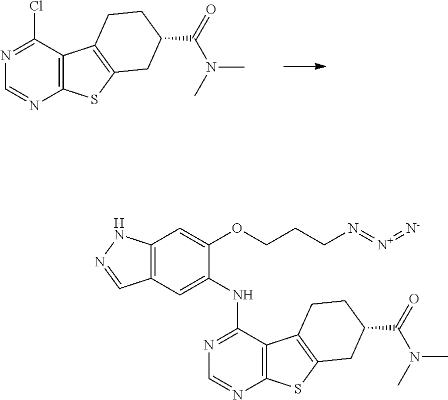 [SMILES: CN(C)C(=O)[C@H]1CCc2c(sc3ncnc(Cl)c23)C1 ; CN(C)C(=O)[C@H]1CCc2c(sc3ncnc(Nc4cc5cn[nH]c5cc4OCCCN=[N+]=[N-])c23)C1]

970 mg (3.28 mmol) (7S)-4-chloro-N,N-dimethyl-5,6,7,8-tetrahydro[1]benzothieno[2,3-d]pyrimidine-7-carboxamide (prepared according to intermediate example 94a) were transformed in analogy to intermediate example 1b using 6-(3-azidopropoxy)-1H-indazol-5-amine (prepared according to intermediate example 110a) to give after working up and purification 937 mg (54%) of the title compound.

¹H-NMR (DMSO-d6): δ=1.82 (1H), 2.01-2.16 (3H), 2.87 (3H), 2.92 (2H), 3.10 (3H), 3.11-3.32 (3H), 3.58 (2H), 4.20 (2H), 7.07 (1H), 7.99 (1H), 8.25 (1H), 8.49 (1H), 8.95 (1H), 12.84 (1H) ppm.

### Example 110a

### 6-(3-Azidopropoxy)-1H-indazol-5-amine

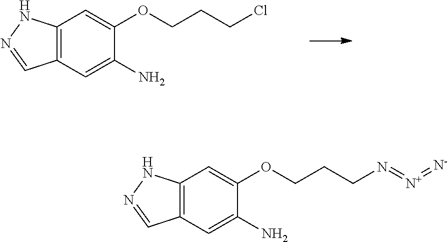 [SMILES: Nc1cc2cn[nH]c2cc1OCCCCl ; [N-]=[N+]=NCCCOc1cc2[nH]ncc2cc1N]

1.22 g (5.41 mmol) 6-(3-chloropropoxy)-1H-indazol-5-amine (prepared according to intermediate example 110b) were transformed in analogy to intermediate example 107a to give after working up and purification 973 mg (78%) of the title compound.

### Example 110b

### 6-(3-Chloropropoxy)-1H-indazol-5-amine

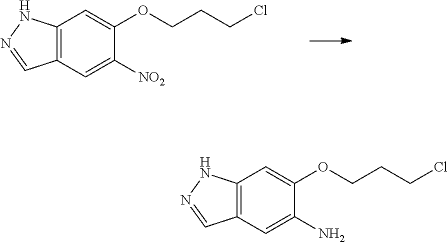 [SMILES: O=[N+]([O-])c1cc2cn[nH]c2cc1OCCCCl ; Nc1cc2cn[nH]c2cc1OCCCCl]

1.49 g (5.81 mmol) 6-(3-chloropropoxy)-5-nitro-1H-indazole (prepared according to intermediate example 110c) were transformed in analogy to intermediate example 94b to give after working up and purification 1.22 g (93%) of the title compound.

### Example 110c

### 6-(3-Chloropropoxy)-5-nitro-1H-indazole

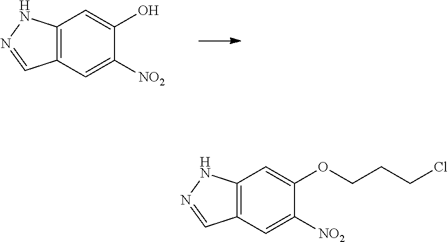 [SMILES: O=[N+]([O-])c1cc2cn[nH]c2cc1O ; O=[N+]([O-])c1cc2cn[nH]c2cc1OCCCCl]

2.50 g (13.96 mmol) 5-nitro-1H-indazol-6-ol (prepared according to intermediate example 94d) were transformed in analogy to intermediate example 94c using 3-chloropropan-1-ol to give after working up and purification 1.49 g (42%) of the title compound.

### Example 111

### (7S)-4-{[6-(3-Aminopropoxy)-1H-indazol-5-yl]amino}-N,N-dimethyl-5,6,7,8-tetrahydro[1]benzothieno[2,3-d]pyrimidine-7-carboxamide

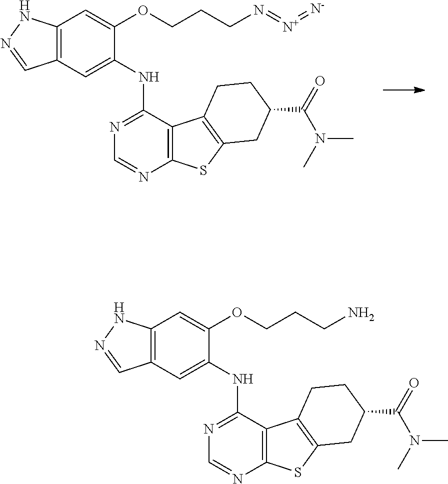 [SMILES: CN(C)C(=O)[C@H]1CCc2c(sc3ncnc(Nc4cc5cn[nH]c5cc4OCCCN=[N+]=[N-])c23)C1 ; CN(C)C(=O)[C@H]1CCc2c(sc3ncnc(Nc4cc5cn[nH]c5cc4OCCCN)c23)C1]

865 mg (1.76 mmol) (7S)-4-{[6-(3-Azidopropoxy)-1H-indazol-5-yl]amino}-N,N-dimethyl-5,6,7,8-tetrahydro[1]benzothieno[2,3-d]pyrimidine-7-carboxamide (prepared according to intermediate example 110) were transformed in analogy to example 108 to give after working up and purification 707 mg (86%) of the title compound.

¹H-NMR (DMSO-d6): δ=1.82 (1H), 1.93 (2H), 2.12 (1H), 2.78 (2H), 2.87 (3H), 2.92 (2H), 3.10 (3H), 3.13-3.26 (3H), 4.22 (2H), 7.07 (1H), 7.99 (1H), 8.26 (1H), 8.50 (1H), 8.98 (1H), 12.84 (1H) ppm.

### Example 112

### (7S)—N,N-Dimethyl-4-({6-[2-(pyrrolidin-1-yl)ethoxy]-1H-indazol-5-yl}amino)-5,6,7,8-tetrahydro[1]benzothieno[2,3-d]pyrimidine-7-carboxamide

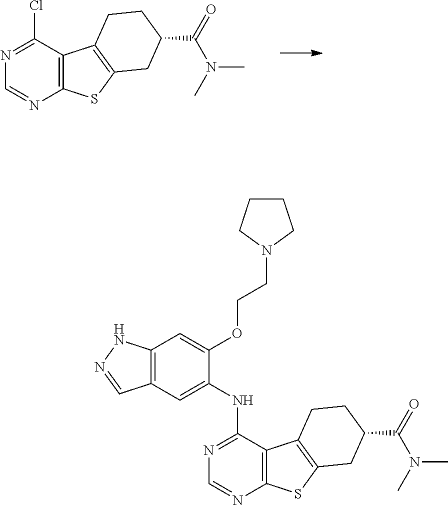 [SMILES: CN(C)C(=O)[C@H]1CCc2c(sc3ncnc(Cl)c23)C1 ; CN(C)C(=O)[C@H]1CCc2c(sc3ncnc(Nc4cc5cn[nH]c5cc4OCCN4CCCC4)c23)C1]

50 mg (169 μmol) (7S)-4-chloro-N,N-dimethyl-5,6,7,8-tetrahydro[1]benzothieno[2,3-d]pyrimidine-7-carboxamide (prepared according to intermediate example 94a) were transformed in analogy to intermediate example 1b using 6-[2-(pyrrolidin-1-yl)ethoxy]-1H-indazol-5-amine (prepared according to intermediate example 112a) to give after working up and purification 78 mg (87%) of the title compound.

¹H-NMR (DMSO-d6): δ=1.69-1.95 (5H), 2.12 (1H), 2.82-3.66 (2H), 2.87 (3H), 3.10 (3H), 3.07-3.29 (9H), 4.48 (2H), 7.15 (1H), 8.01 (1H), 8.19 (1H), 8.42 (1H), 8.66 (1H), 12.95 (1H) ppm.

### Example 112a

### 6-[2-(Pyrrolidin-1-yl)ethoxy]-1H-indazol-5-amine

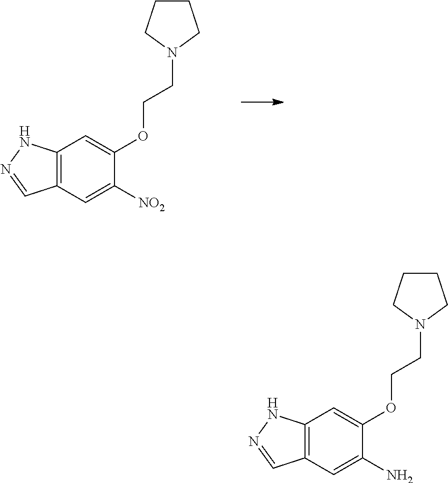 [SMILES: O=[N+]([O-])c1cc2cn[nH]c2cc1OCCN1CCCC1 ; Nc1cc2cn[nH]c2cc1OCCN1CCCC1]

533 mg (1.93 mmol) 5-nitro-6-[2-(pyrrolidin-1-yl)ethoxy]-1H-indazole (prepared according to intermediate example 112b) were transformed in analogy to intermediate example 94b to give after working up and purification 478 mg (100%) of the title compound.

### Example 112b

### 5-Nitro-6-[2-(pyrrolidin-1-yl)ethoxy]-1H-indazole

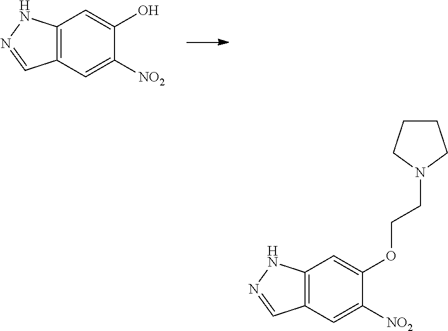 [SMILES: O=[N+]([O-])c1cc2cn[nH]c2cc1O ; O=[N+]([O-])c1cc2cn[nH]c2cc1OCCN1CCCC1]

1.00 g (5.58 mmol) 5-nitro-1H-indazol-6-ol (prepared according to intermediate example 94d) were transformed in analogy to intermediate example 94c using 2-(pyrrolidin-1-yl)ethanol to give after working up and purification 542 mg (35%) of the title compound.

### Example 113

### (7S)—N,N-Dimethyl-4-({6-[2-(piperidin-1-yl)ethoxy]-1H-indazol-5-yl}amino)-5,6,7,8-tetrahydro[1]benzothieno[2,3-d]pyrimidine-7-carboxamide

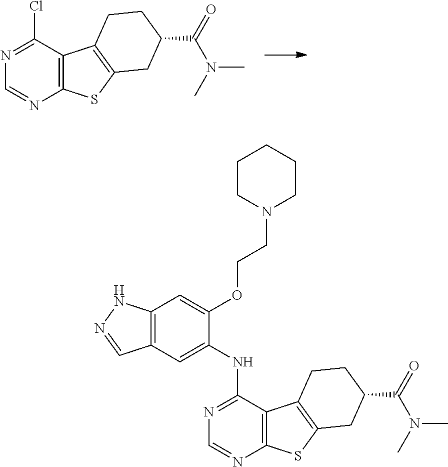 [SMILES: CN(C)C(=O)[C@H]1CCc2c(sc3ncnc(Cl)c23)C1 ; CN(C)C(=O)[C@H]1CCc2c(sc3ncnc(Nc4cc5cn[nH]c5cc4OCCN4CCCCC4)c23)C1]

50 mg (169 μmol) (7S)-4-chloro-N,N-dimethyl-5,6,7,8-tetrahydro[1]benzothieno[2,3-d]pyrimidine-7-carboxamide (prepared according to intermediate example 94a) were transformed in analogy to intermediate example 1b using 6-[2-(piperidin-1-yl)ethoxy]-1H-indazol-5-amine (prepared according to intermediate example 113a) to give after working up and purification 41.1 mg (44%) of the title compound.

¹H-NMR (DMSO-d6): δ=1.35 (2H), 1.54 (4H), 1.83 (1H), 2.11 (1H), 2.31-2.59 (4H), 2.88 (3H), 2.94 (2H), 3.10 (3H), 3.13-3.56 (5H), 4.41 (2H), 7.15 (1H), 8.00 (1H), 8.17 (1H), 8.43 (1H), 8.87 (1H), 12.89 (1H) ppm.

### Example 113a

### 6-[2-(Piperidin-1-yl)ethoxy]-1H-indazol-5-amine

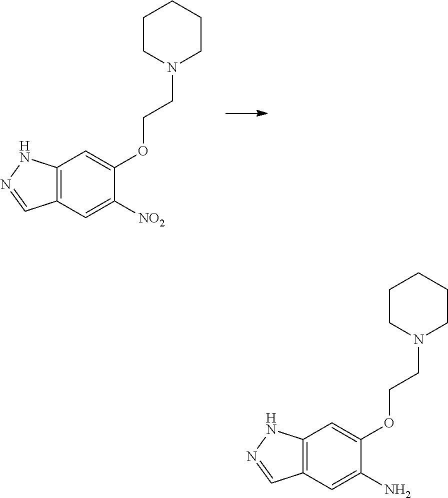 [SMILES: O=[N+]([O-])c1cc2cn[nH]c2cc1OCCN1CCCCC1 ; Nc1cc2cn[nH]c2cc1OCCN1CCCCC1]

925 mg (3.19 mmol) 5-nitro-6-[2-(piperidin-1-yl)ethoxy]-1H-indazole (prepared according to intermediate example 113b) were transformed in analogy to intermediate example 94b to give after working up and purification 588 mg (71%) of the title compound.

### Example 113b

### 5-Nitro-6-[2-(piperidin-1-yl)ethoxy]-1H-indazole

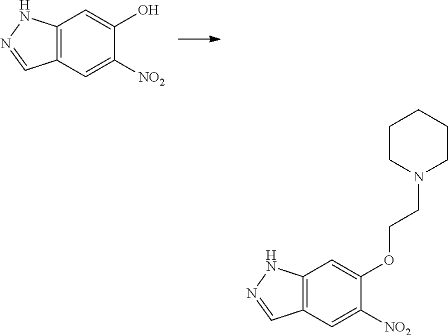 [SMILES: O=[N+]([O-])c1cc2cn[nH]c2cc1O ; O=[N+]([O-])c1cc2cn[nH]c2cc1OCCN1CCCCC1]

1.00 g (5.58 mmol) 5-nitro-1H-indazol-6-ol (prepared according to intermediate example 94d) were transformed in analogy to intermediate example 94c using 2-(piperidin-1-yl)ethanol to give after working up and purification 931 mg (57%) of the title compound.

### Example 114

### [(7S)-4-({6-[2-(Dimethylamino)ethoxy]-1H-indazol-5-yl}amino)-5,6,7,8-tetrahydro[1]benzothieno[2,3-d]pyrimidin-7-yl][(3R)-3-methylmorpholin-4-yl]methanone

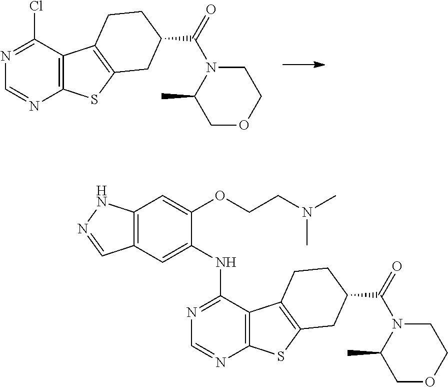 [SMILES: C[C@@H]1COCCN1C(=O)[C@H]1CCc2c(sc3ncnc(Cl)c23)C1 ; C[C@@H]1COCCN1C(=O)[C@H]1CCc2c(sc3ncnc(Nc4cc5cn[nH]c5cc4OCCN(C)C)c23)C1]

50 mg (142 μmol) [(7S)-4-chloro-5,6,7,8-tetrahydro[1]benzothieno[2,3-d]pyrimidin-7-yl][(3R)-3-methylmorpholin-4-yl]methanone (prepared according to intermediate example 114a) were transformed in analogy to intermediate example 1b using 6-[2-(dimethylamino)ethoxy]-1H-indazol-5-amine (prepared according to intermediate example 109a) to give after working up and purification 18.1 mg (23%) of the title compound.

¹H-NMR (DMSO-d6): δ=1.16+1.29 (3H), 1.94 (1H), 2.10 (1H), 2.21 (6H), 2.72 (2H), 2.93 (2H), 3.08-3.90 (8H), 4.03-4.49 (4H), 7.14 (1H), 7.99 (1H), 8.26 (1H), 8.50 (1H), 8.97 (1H), 12.83 (1H) ppm.

### Example 114a

### [(7S)-4-chloro-5,6,7,8-tetrahydro[1]benzothieno[2,3-d]pyrimidin-7-yl][(3R)-3-methylmorpholin-4-yl]methanone

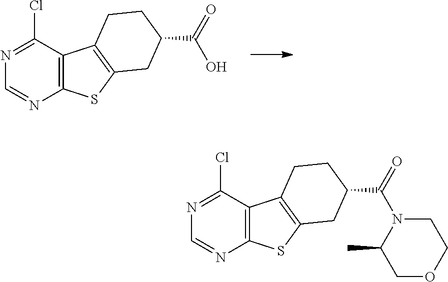 [SMILES: O=C(O)[C@H]1CCc2c(sc3ncnc(Cl)c23)C1 ; C[C@@H]1COCCN1C(=O)[C@H]1CCc2c(sc3ncnc(Cl)c23)C1]

500 mg (1.86 mmol) (7S)-4-chloro-5,6,7,8-tetrahydro[1]benzothieno[2,3-d]pyrimidine-7-carboxylic acid (prepared according to intermediate example 66b) were transformed in analogy to example 1 using (3R)-3-methylmorpholine to give after working up and purification 652 mg (99%) of the title compound.

### Example 115

### [(3R)-3-Methylmorpholin-4-yl][(7S)-4-({6-[2-(pyrrolidin-1-yl)ethoxy]-1H-indazol-5-yl}amino)-5,6,7,8-tetrahydro[1]benzothieno[2,3-d]pyrimidin-7-yl]methanone

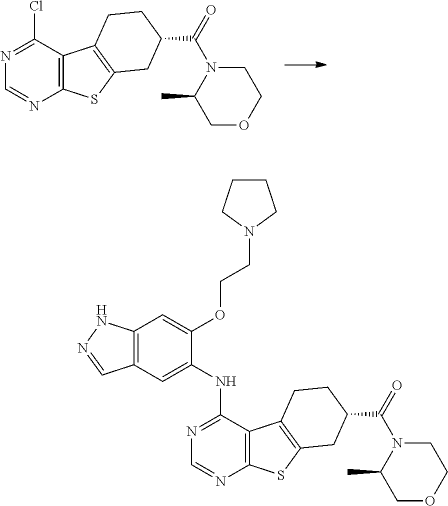 [SMILES: C[C@@H]1COCCN1C(=O)[C@H]1CCc2c(sc3ncnc(Cl)c23)C1 ; C[C@@H]1COCCN1C(=O)[C@H]1CCc2c(sc3ncnc(Nc4cc5cn[nH]c5cc4OCCN4CCCC4)c23)C1]

50 mg (142 μmol) [(7S)-4-chloro-5,6,7,8-tetrahydro[1]benzothieno[2,3-d]pyrimidin-7-yl][(3R)-3-methylmorpholin-4-yl]methanone (prepared according to intermediate example 114a) were transformed in analogy to intermediate example 1b using 6-[2-(pyrrolidin-1-yl)ethoxy]-1H-indazol-5-amine (prepared according to intermediate example 112a) to give after working up and purification 32.8 mg (39%) of the title compound.

¹H-NMR (DMSO-d6): δ=1.16+1.29 (3H), 1.70 (4H), 1.93 (1H), 2.08 (1H), 2.37-2.76 (4H), 2.81-3.92 (12H), 4.05-4.50 (4H), 7.14 (1H), 8.00 (1H), 8.25 (1H), 8.47 (1H), 8.85 (1H), 12.86 (1H) ppm.

### Example 116

### [(3R)-3-Methylmorpholin-4-yl][(7S)-4-({6-[2-(piperidin-1-yl)ethoxy]-1H-indazol-5-yl}amino)-5,6,7,8-tetrahydro[1]benzothieno[2,3-d]pyrimidin-7-yl]methanone

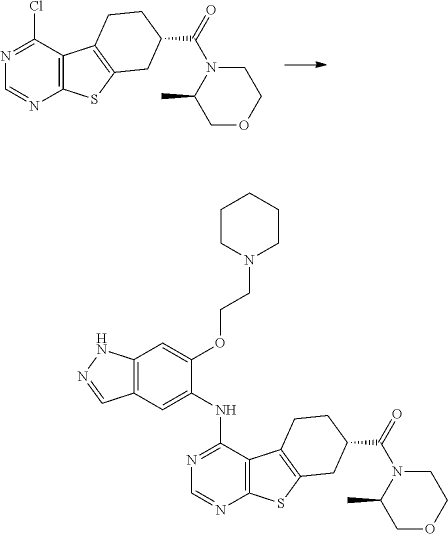 [SMILES: C[C@@H]1COCCN1C(=O)[C@H]1CCc2c(sc3ncnc(Cl)c23)C1 ; C[C@@H]1COCCN1C(=O)[C@H]1CCc2c(sc3ncnc(Nc4cc5cn[nH]c5cc4OCCN4CCCCC4)c23)C1]

50 mg (142 μmol) [(7S)-4-chloro-5,6,7,8-tetrahydro[1]benzothieno[2,3-d]pyrimidin-7-yl][(3R)-3-methylmorpholin-4-yl]methanone (prepared according to intermediate example 114a) were transformed in analogy to intermediate example 1b using 6-[2-(piperidin-1-yl)ethoxy]-1H-indazol-5-amine (prepared according to intermediate example 113a) to give after working up and purification 27.8 mg (32%) of the title compound.

¹H-NMR (DMSO-d6): δ=1.10-1.50 (9H), 1.93 (1H), 2.08 (1H), 2.35-2.44 (4H), 2.75 (2H), 2.86-3.90 (10H), 4.02-4.50 (4H), 7.14 (1H), 7.99 (1H), 8.26 (1H), 8.49 (1H), 8.92 (1H), 12.83 (1H) ppm.

### Example 117

### [(7S)-4-({6-[2-(Dimethylamino)ethoxy]-1H-indazol-5-yl}amino)-5,6,7,8-tetrahydro[1]benzothieno[2,3-d]pyrimidin-7-yl][(3S)-3-methylmorpholin-4-yl]methanone

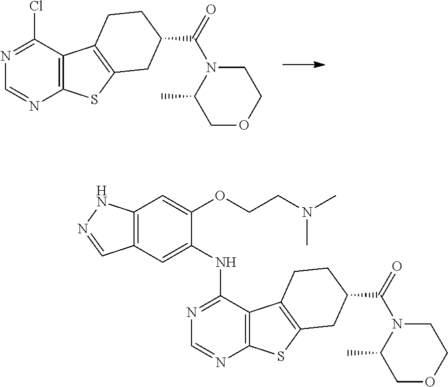 [SMILES: C[C@H]1COCCN1C(=O)[C@H]1CCc2c(sc3ncnc(Cl)c23)C1 ; C[C@H]1COCCN1C(=O)[C@H]1CCc2c(sc3ncnc(Nc4cc5cn[nH]c5cc4OCCN(C)C)c23)C1]

50 mg (142 μmol) [(7S)-4-chloro-5,6,7,8-tetrahydro[1]benzothieno[2,3-d]pyrimidin-7-yl][(3S)-3-methylmorpholin-4-yl]methanone (prepared according to intermediate example 117a) were transformed in analogy to intermediate example 1b using 6-[2-(dimethylamino)ethoxy]-1H-indazol-5-amine (prepared according to intermediate example 109a) to give after working up and purification 53.3 mg (67%) of the title compound.

¹H-NMR (DMSO-d6): δ=1.16+1.30 (3H), 1.82 (1H), 2.09 (1H), 2.45 (4H), 2.80-3.56 (9H), 3.38-3.56 (3H), 3.65 (1H), 3.72-4.51 (5H), 7.16 (1H), 8.00 (1H), 8.19 (1H), 8.47 (1H), 8.83+8.87 (1H), 12.90 (1H) ppm.

### Example 117a

### [(7S)-4-chloro-5,6,7,8-tetrahydro[1]benzothieno[2,3-d]pyrimidin-7-yl][(3S)-3-methylmorpholin-4-yl]methanone

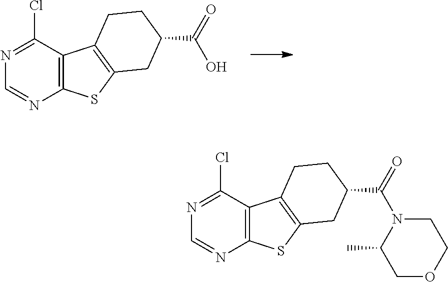 [SMILES: O=C(O)[C@H]1CCc2c(sc3ncnc(Cl)c23)C1 ; C[C@H]1COCCN1C(=O)[C@H]1CCc2c(sc3ncnc(Cl)c23)C1]

400 mg (1.49 mmol) (7S)-4-chloro-5,6,7,8-tetrahydro[1]benzothieno[2,3-d]pyrimidine-7-carboxylic acid (prepared according to intermediate example 66b) were transformed in analogy to example 1 using (3S)-3-methylmorpholine to give after working up and purification 508 mg (97%) of the title compound.

### Example 118

### [(3S)-3-Methylmorpholin-4-yl][(7S)-4-({6-[2-(pyrrolidin-1-yl)ethoxy]-1H-indazol-5-yl}amino)-5,6,7,8-tetrahydro[1]benzothieno[2,3-d]pyrimidin-7-yl]methanone

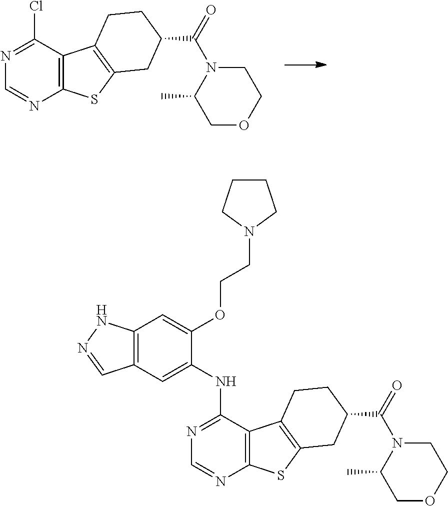 [SMILES: C[C@H]1COCCN1C(=O)[C@H]1CCc2c(sc3ncnc(Cl)c23)C1 ; C[C@H]1COCCN1C(=O)[C@H]1CCc2c(sc3ncnc(Nc4cc5cn[nH]c5cc4OCCN4CCCC4)c23)C1]

50 mg (142 μmol) [(7S)-4-chloro-5,6,7,8-tetrahydro[1]benzothieno[2,3-d]pyrimidin-7-yl][(3S)-3-methylmorpholin-4-yl]methanone (prepared according to intermediate example 117a) were transformed in analogy to intermediate example 1b using 6-[2-(pyrrolidin-1-yl)ethoxy]-1H-indazol-5-amine (prepared according to intermediate example 112a) to give after working up and purification 66.0 mg (79%) of the title compound.

¹H-NMR (DMSO-d6): δ=1.16+1.30 (3H), 1.64-1.95 (5H), 2.06 (1H), 2.79-3.58 (13H), 3.66 (1H), 3.73-4.58 (6H), 7.15 (1H), 8.01 (1H), 8.20 (1H), 8.44 (1H), 8.71 (1H), 12.93 (1H) ppm.

### Example 119

### [(3S)-3-Methylmorpholin-4-yl][(7S)-4-({6-[2-(piperidin-1-yl)ethoxy]-1H-indazol-5-yl}amino)-5,6,7,8-tetrahydro[1]benzothieno[2,3-d]pyrimidin-7-yl]methanone

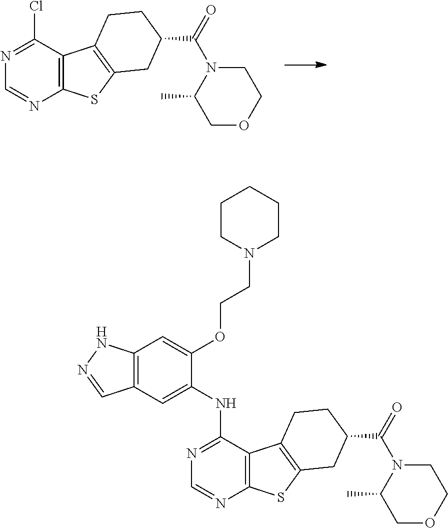 [SMILES: C[C@H]1COCCN1C(=O)[C@H]1CCc2c(sc3ncnc(Cl)c23)C1 ; C[C@H]1COCCN1C(=O)[C@H]1CCc2c(sc3ncnc(Nc4cc5cn[nH]c5cc4OCCN4CCCCC4)c23)C1]

50 mg (142 μmol) [(7S)-4-chloro-5,6,7,8-tetrahydro[1]benzothieno[2,3-d]pyrimidin-7-yl][(3S)-3-methylmorpholin-4-yl]methanone (prepared according to intermediate example 117a) were transformed in analogy to intermediate example 1b using 6-[2-(piperidin-1-yl)ethoxy]-1H-indazol-5-amine (prepared according to intermediate example 113a) to give after working up and purification 35.4 mg (41%) of the title compound.

¹H-NMR (DMSO-d6): δ=1.09-1.55 (9H), 1.84 (1H), 2.08 (1H), 2.40 (4H), 2.68-3.56 (9H), 3.66 (1H), 3.73-4.50 (6H), 7.14 (1H), 7.99 (1H), 8.26 (1H), 8.49 (1H), 8.91 (1H), 12.84 (1H) ppm.

### Example 120

### {(7S)-4-[(6-Methoxy-1H-indazol-5-yl)amino]-5,6,7,8-tetrahydro[1]benzothieno[2,3-d]pyrimidin-7-yl}[(1S,4S)-5-methyl-2,5-diazabicyclo[2.2.1]hept-2-yl]methanone

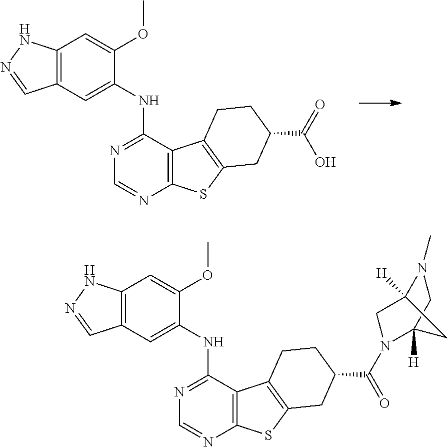 [SMILES: COc1cc2[nH]ncc2cc1Nc1ncnc2sc3c(c12)CC[C@H](C(=O)O)C3 ; COc1cc2[nH]ncc2cc1Nc1ncnc2sc3c(c12)CC[C@H](C(=O)N1C[C@@H]2C[C@H]1CN2C)C3]

100 mg (253 μmol) (7S)-4-[(6-methoxy-1H-indazol-5-yl)amino]-5,6,7,8-tetrahydro[1]benzothieno[2,3-d]pyrimidine-7-carboxylic acid (prepared according to intermediate example 20a) were transformed in analogy to example 1 using (1S,4S)-2-methyl-2,5-diazabicyclo[2.2.1]heptane to give after working up and purification 9.6 mg (7%) of the title compound.

¹H-NMR (DMSO-d6): δ=1.64 (1H), 1.75-1.95 (2H), 2.13 (1H), 2.32 (3H), 2.40-3.70 (10H), 3.98 (3H), 4.53+4.60 (1H), 7.09 (1H), 7.99 (1H), 8.22 (1H), 8.46 (1H), 8.75+8.78 (1H), 12.85 (1H) ppm.

### Example 121

### {(7S)-4-[(6-Methoxy-1H-indazol-5-yl)amino]-5,6,7,8-tetrahydro[1]benzothieno[2,3-d]pyrimidin-7-yl}[(1R,4R)-5-methyl-2,5-diazabicyclo[2.2.1]hept-2-yl]methanone

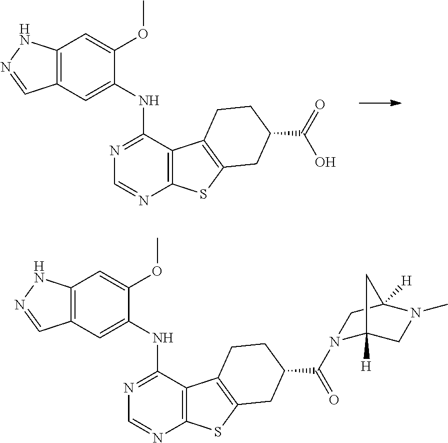

100 mg (253 μmol) (7S)-4-[(6-methoxy-1H-indazol-5-yl)amino]-5,6,7,8-tetrahydro[1]benzothieno[2,3-d]pyrimidine-7-carboxylic acid (prepared according to intermediate example 20a) were transformed in analogy to example 1 using (1R,4R)-2-methyl-2,5-diazabicyclo[2.2.1]heptane to give after working up and purification 13.8 mg (11%) of the title compound.

¹H-NMR (DMSO-d6): δ=1.56-1.91 (3H), 2.13 (1H), 2.29+2.33 (3H), 2.39-3.67 (10H), 3.99 (3H), 4.54+4.60 (1H), 7.09 (1H), 8.00 (1H), 8.23 (1H), 8.46 (1H), 8.79 (1H), 12.85 (1H) ppm.

### Example 122

### [4-(Dimethylamino)piperidin-1-yl]{(7S)-4-[(6-ethoxy-1H-indazol-5-yl)amino]-5,6,7,8-tetrahydro[1]benzothieno[2,3-d]pyrimidin-7-yl}methanone

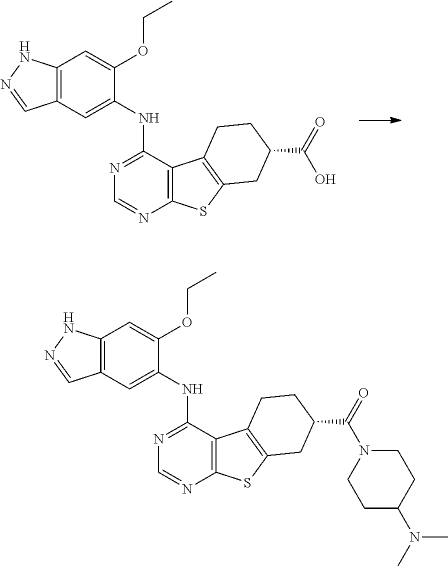 [SMILES: CCOc1cc2[nH]ncc2cc1Nc1ncnc2sc3c(c12)CC[C@H](C(=O)O)C3 ; CCOc1cc2[nH]ncc2cc1Nc1ncnc2sc3c(c12)CC[C@H](C(=O)N1CCC(N(C)C)CC1)C3]

430 mg (1.05 mmol) (7S)-4-[(6-ethoxy-1H-indazol-5-yl)amino]-5,6,7,8-tetrahydro[1]benzothieno[2,3-d]pyrimidine-7-carboxylic acid (prepared according to intermediate example 65a) were transformed in analogy to example 1 using N,N-dimethylpiperidin-4-amine to give after working up and purification 255 mg (44%) of the title compound.

¹H-NMR (DMSO-d6): δ=1.20 (1H), 1.13 (1H), 1.46 (3H), 1.72-1.93 (3H), 2.04 (1H), 2.17 (6H), 2.32 (1H), 2.61 (1H), 2.84-2.98 (2H), 3.07 (1H), 3.13-3.26 (3H), 4.05 (1H), 4.19 (2H), 4.40 (1H), 7.04 (1H), 7.98 (1H), 8.34 (1H), 8.51 (1H), 9.01 (1H), 12.80 (1H) ppm.

### Example 123

### (7S)-4-[(6-Methoxy-1H-indazol-5-yl)amino]-N-methyl-N-(3, 3,3-trifluoropropyl)-5,6,7,8-tetrahydro[1]benzothieno[2,3-d]pyrimidine-7-carboxamide

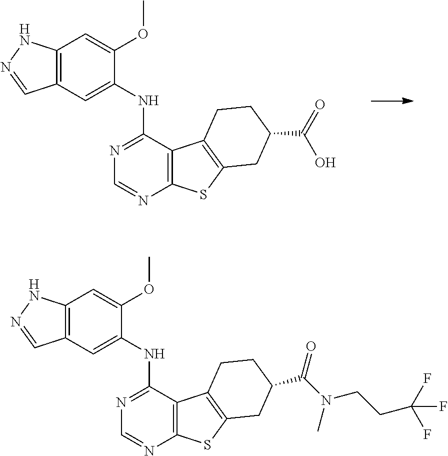 [SMILES: COc1cc2[nH]ncc2cc1Nc1ncnc2sc3c(c12)CC[C@H](C(=O)O)C3 ; COc1cc2[nH]ncc2cc1Nc1ncnc2sc3c(c12)CC[C@H](C(=O)N(C)CCC(F)(F)F)C3]

100 mg (253 μmol) (7S)-4-[(6-methoxy-1H-indazol-5-yl)amino]-5,6,7,8-tetrahydro[1]benzothieno[2,3-d]pyrimidine-7-carboxylic acid (prepared according to intermediate example 20a) were transformed in analogy to example 1 using 3,3,3-trifluoro-N-methylpropan-1-amine to give after working up and purification 83.2 mg (62%) of the title compound.

¹H-NMR (DMSO-d6): δ=1.83 (1H), 2.14 (1H), 2.42-2.64 (2H), 2.88+3.13 (3H), 2.92 (2H), 3.08-3.26 (3H), 3.44-3.70 (2H), 3.98 (3H), 7.09 (1H), 7.99 (1H), 8.21 (1H), 8.46 (1H), 8.78 (1H), 12.84 (1H) ppm.

### Example 124

### (7S)—N-(2-Hydroxy-2-methylpropyl)-4-[(6-methoxy-1H-indazol-5-yl)amino]-N-methyl-5,6,7,8-tetrahydro[1]benzothieno[2,3-d]pyrimidine-7-carboxamide

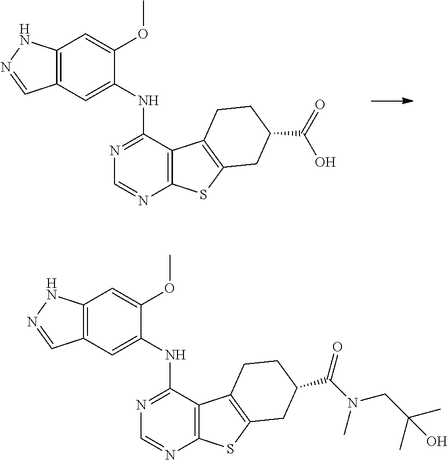 [SMILES: COc1cc2[nH]ncc2cc1Nc1ncnc2sc3c(c12)CC[C@H](C(=O)O)C3 ; COc1cc2[nH]ncc2cc1Nc1ncnc2sc3c(c12)CC[C@H](C(=O)N(C)CC(C)(C)O)C3]

50 mg (126 μmol) (7S)-4-[(6-methoxy-1H-indazol-5-yl)amino]-5,6,7,8-tetrahydro[1]benzothieno[2,3-d]pyrimidine-7-carboxylic acid (prepared according to intermediate example 20a) were transformed in analogy to example 1 using 2-methyl-1-(methylamino)propan-2-ol to give after working up and purification 31.7 mg (50%) of the title compound.

¹H-NMR (DMSO-d6): δ=1.02-1.18 (6H), 1.85 (1H), 2.14 (1H), 2.83-3.02 (2H), 2.95+3.22 (3H), 3.17-3.37 (4H), 3.45 (1H), 3.97+3.99 (3H), 4.49+4.55 (1H), 7.09 (1H), 7.99 (1H), 8.22 (1H), 8.45+8.46 (1H), 8.76+8.80 (1H), 12.84 (1H) ppm.

### Example 125

### (7S)—N-Methyl-4-{[6-(propan-2-yloxy)-1H-indazol-5-yl]amino}-N-(3,3,3-trifluoropropyl)-5,6,7,8-tetrahydro[1]benzothieno[2,3-d]pyrimidine-7-carboxamide

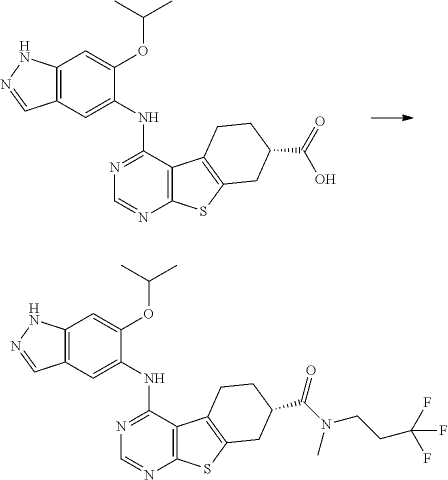 [SMILES: CC(C)Oc1cc2[nH]ncc2cc1Nc1ncnc2sc3c(c12)CC[C@H](C(=O)O)C3 ; CC(C)Oc1cc2[nH]ncc2cc1Nc1ncnc2sc3c(c12)CC[C@H](C(=O)N(C)CCC(F)(F)F)C3]

100 mg (236 μmol) (7S)-4-[(6-isopropoxy-1H-indazol-5-yl)amino]-5,6,7,8-tetrahydro[1]benzothieno[2,3-d]pyrimidine-7-carboxylic acid (prepared according to intermediate example 78a) were transformed in analogy to example 1 using 3,3,3-trifluoro-N-methylpropan-1-amine to give after working up and purification 78.5 mg (59%) of the title compound.

¹H-NMR (DMSO-d6): δ=1.41 (6H), 1.87 (1H), 2.08 (1H), 2.42-2.59 (2H), 2.87+3.12 (3H), 2.93 (2H), 3.08-3.37 (3H), 3.44-3.74 (2H), 4.88 (1H), 7.11 (1H), 7.98 (1H), 8.36 (1H), 8.53 (1H), 9.07 (1H), 12.75 (1H) ppm.

### Example 126

### (7S)—N-(2-Hydroxy-2-methylpropyl)-N-methyl-4-{[6-(propan-2-yloxy)-1H-indazol-5-yl]amino}-5,6,7,8-tetrahydro[1]benzothieno[2,3-d]pyrimidine-7-carboxamide

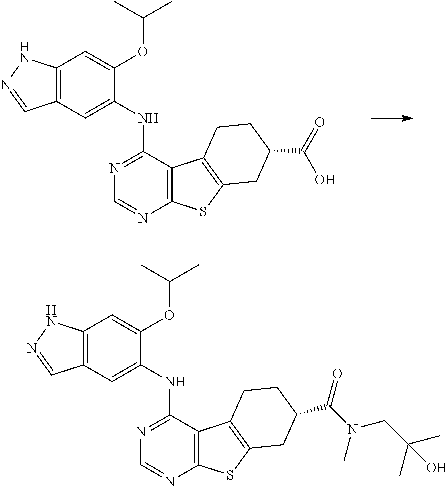 [SMILES: CC(C)Oc1cc2[nH]ncc2cc1Nc1ncnc2sc3c(c12)CC[C@H](C(=O)O)C3 ; CC(C)Oc1cc2[nH]ncc2cc1Nc1ncnc2sc3c(c12)CC[C@H](C(=O)N(C)CC(C)(C)O)C3]

50 mg (118 μmol) (7S)-4-[(6-isopropoxy-1H-indazol-5-yl)amino]-5,6,7,8-tetrahydro[1]benzothieno[2,3-d]pyrimidine-7-carboxylic acid (prepared according to intermediate example 78a) were transformed in analogy to example 1 using 2-methyl-1-(methylamino)propan-2-ol to give after working up and purification 45.8 mg (72%) of the title compound.

¹H-NMR (DMSO-d6): δ=1.02-1.19 (6H), 1.37-1.47 (6H), 1.89 (1H), 2.10 (1H), 2.82-3.05 (2H), 2.95+3.21 (3H), 3.16-3.45 (5H), 4.50+4.54 (1H), 4.89 (1H), 7.11 (1H), 7.99 (1H), 8.37 (1H), 8.53 (1H), 9.06+9.07 (1H), 12.75 (1H) ppm.

### Example 127

### (7S)-4-(1H-Indazol-5-ylamino)-N,N-di(propan-2-yl)-5,6,7,8-tetrahydro[1]benzothieno[2,3-d]pyrimidine-7-carboxamide

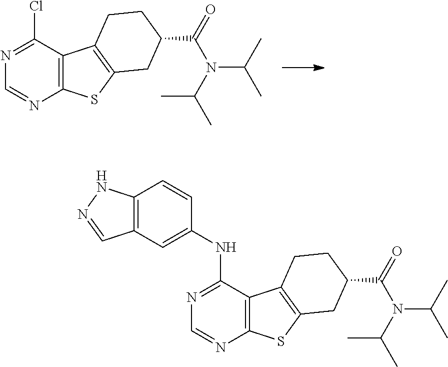 [SMILES: CC(C)N(C(=O)[C@H]1CCc2c(sc3ncnc(Cl)c23)C1)C(C)C ; CC(C)N(C(=O)[C@H]1CCc2c(sc3ncnc(Nc4ccc5[nH]ncc5c4)c23)C1)C(C)C]

65 mg (185 μmol) (7S)-4-chloro-N,N-diisopropyl-5,6,7,8-tetrahydro[1]benzothieno[2,3-d]pyrimidine-7-carboxamide (prepared according to intermediate example 127a) were transformed in analogy to intermediate example 1b to give after working up and purification 62.9 mg (72%) of the title compound.

¹H-NMR (DMSO-d6): δ=1.20 (6H), 1.30 (6H), 1.81 (1H), 2.00 (1H), 2.83 (1H), 2.94-3.08 (2H), 3.18-3.28 (2H), 3.59 (1H), 4.11 (1H), 7.47-7.53 (2H), 7.98 (1H), 8.04 (1H), 8.17 (1H), 8.30 (1H), 13.00 (1H) ppm.

### Example 127a

### (7S)-4-Chloro-N,N-diisopropyl-5,6,7,8-tetrahydro[1]benzothieno[2,3-d]pyrimidine-7-carboxamide

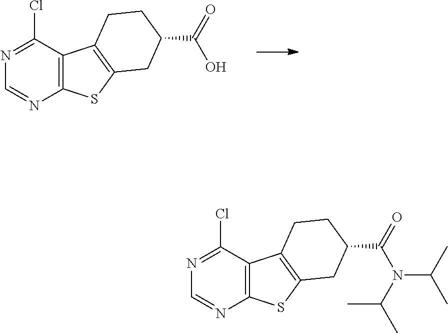 [SMILES: O=C(O)[C@H]1CCc2c(sc3ncnc(Cl)c23)C1 ; CC(C)N(C(=O)[C@H]1CCc2c(sc3ncnc(Cl)c23)C1)C(C)C]

200 mg (744 μmol) (7S)-4-chloro-5,6,7,8-tetrahydro[1]benzothieno[2,3-d]pyrimidine-7-carboxylic acid (prepared according to intermediate example 66b) were transformed in analogy to example 1 using N-isopropylpropan-2-amine to give after working up and purification 65 mg (25%) of the title compound.

### Example 128

### (7S)—N,N-Dimethyl-4-({6-[methyl(prop-2-en-1-yl)amino]-1H-indazol-5-yl}amino)-5,6,7,8-tetrahydro[1]benzothieno[2,3-d]pyrimidine-7-carboxamide

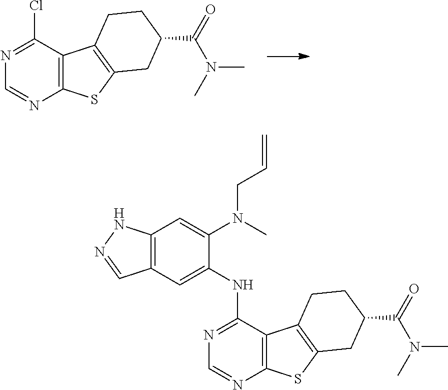 [SMILES: CN(C)C(=O)[C@H]1CCc2c(sc3ncnc(Cl)c23)C1 ; C=CCN(C)c1cc2[nH]ncc2cc1Nc1ncnc2sc3c(c12)CC[C@H](C(=O)N(C)C)C3]

168.2 mg (569 μmol) (7S)-4-chloro-N,N-dimethyl-5,6,7,8-tetrahydro[1]benzothieno[2,3-d]pyrimidine-7-carboxamide (prepared according to intermediate example 94a) were transformed in analogy to intermediate example 1b using N⁶-allyl-N⁶-methyl-1H-indazole-5,6-diamine (prepared according to intermediate example 128a) to give after working up and purification 34.4 mg (13%) of the title compound.

¹H-NMR (DMSO-d6): δ=1.85 (1H), 2.13 (1H), 2.64 (3H), 2.80 (3H), 2.93 (2H), 3.11 (3H), 3.13-3.27 (3H), 3.64 (2H), 5.16 (1H), 5.23 (1H), 5.86 (1H), 7.43 (1H), 8.03 (1H), 8.52 (1H), 9.02 (1H), 9.22 (1H), 12.89 (1H) ppm.

### Example 128a

### N⁶-Allyl-N⁶-methyl-1H-indazole-5,6-diamine

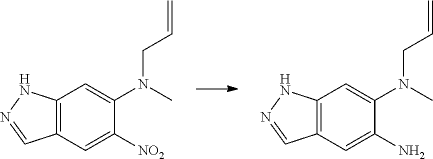 [SMILES: C=CCN(C)c1cc2[nH]ncc2cc1[N+](=O)[O-] ; C=CCN(C)c1cc2[nH]ncc2cc1N]

A mixture comprising 609 mg (2.62 mmol)N-allyl-N-methyl-5-nitro-1H-indazol-6-amine (prepared according to intermediate example 128b), 1.46 g iron powder, 70 mg ammonium chloride, 25 mL ethanol and 6 mL water was stirred vigorously at 90° C. for 6 hours. After filtration most of the ethanol was removed and the mixture extracted with ethyl acetate. The organic layer was washed with water, brine and dried over sodium sulphate to give after filtration and removal of the solvent 498 mg (94%) of the title compound that was used without further purification.

### Example 128b

### N-Allyl-N-methyl-5-nitro-1H-indazol-6-amine

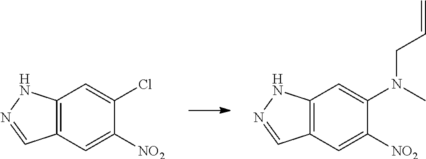 [SMILES: O=[N+]([O-])c1cc2cn[nH]c2cc1Cl ; C=CCN(C)c1cc2[nH]ncc2cc1[N+](=O)[O-]]

500 mg (2.53 mmol) 6-chloro-5-nitro-1H-indazole (CAS-No: 101420-98-8) were transformed in analogy to intermediate example 99b using N-methylprop-2-en-1-amine to give after working up and purification 515 mg (88%) of the title compound.

### Example 129

### (7S)-4-{[4-Fluoro-6-(propan-2-yloxy)-1H-indazol-5-yl]amino}-N,N-dimethyl-5,6,7,8-tetrahydro[1]benzothieno[2,3-d]pyrimidine-7-carboxamide

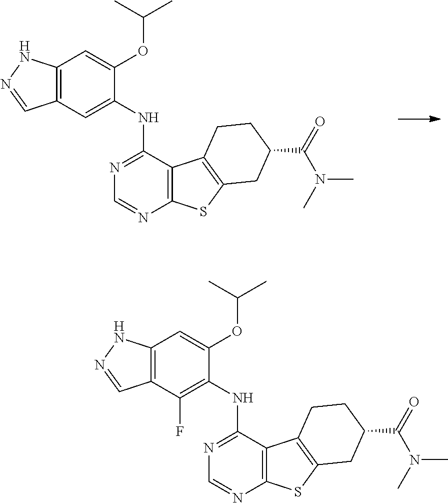 [SMILES: CC(C)Oc1cc2[nH]ncc2cc1Nc1ncnc2sc3c(c12)CC[C@H](C(=O)N(C)C)C3 ; CC(C)Oc1cc2[nH]ncc2c(F)c1Nc1ncnc2sc3c(c12)CC[C@H](C(=O)N(C)C)C3]

A mixture comprising 74 mg (164 μmol) (7S)-4-[(6-isopropoxy-1H-indazol-5-yl)amino]-N,N-dimethyl-5,6,7,8-tetrahydro[1]benzothieno[2,3-d]pyrimidine-7-carboxamide (prepared according to intermediate example 78), 22 mL N,N-dimethylformamide and 58.2 mg 1-chloromethyl-4-fluoro-1,4-diazoniabicyclo[2.2.2]octane bis(tetrafluoroborate) was stirred at 23° C. for 23 hours. The solvent was removed and the residue purified by chromatography to give 19.4 mg (23%) of the title compound.

¹H-NMR (DMSO-d6): δ=1.19 (6H), 1.80 (1H), 2.06 (1H), 2.87 (3H), 2.92 (2H), 3.09 (3H), 3.04-3.28 (3H), 4.62 (1H), 6.90 (1H), 7.61 (1H), 8.07 (1H), 8.17 (1H), 13.10 (1H) ppm.

### Example 130

### [(7S)-4-{[6-(2-Aminoethoxy)-1H-indazol-5-yl]amino}-5,6,7,8-tetrahydro[1]benzothieno[2,3-d]pyrimidin-7-yl][(3R)-3-methylmorpholin-4-yl]methanone

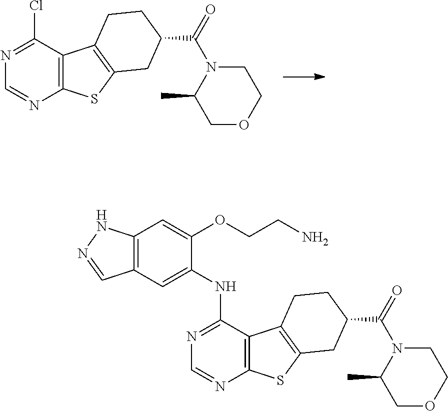 [SMILES: C[C@@H]1COCCN1C(=O)[C@H]1CCc2c(sc3ncnc(Cl)c23)C1 ; C[C@@H]1COCCN1C(=O)[C@H]1CCc2c(sc3ncnc(Nc4cc5cn[nH]c5cc4OCCN)c23)C1]

A mixture comprising 50 mg (142 μmol) [(7S)-4-chloro-5,6,7,8-tetrahydro[1]benzothieno[2,3-d]pyrimidin-7-yl][(3R)-3-methylmorpholin-4-yl]methanone (prepared according to intermediate example 114a), 43.6 mg tert-butyl {2-[(5-amino-1H-indazol-6-yl)oxy]ethyl}carbamate (prepared according to intermediate example 130a) and 2 mL ethanol was heated at reflux overnight. 107 μL hydrochloric acid (4N in dioxane) were added and stirring was continued at 23° C. overnight. The solvents were removed and the residue was purified by chromatography to give 33.7 mg (44%) of the title compound.

¹H-NMR (DMSO-d6): δ=1.15+1.32 (3H), 1.91 (1H), 2.10 (1H), 2.81-3.77 (13H), 3.85 (1H), 4.09-4.46 (3H), 7.07 (1H), 8.00 (1H), 8.31 (1H), 8.36 (1H), 8.51 (1H), 8.99 (1H), 12.83 (1H) ppm.

### Example 130a

### tert-Butyl {2-[(5-amino-1H-indazol-6-yl)oxy]ethyl}carbamate

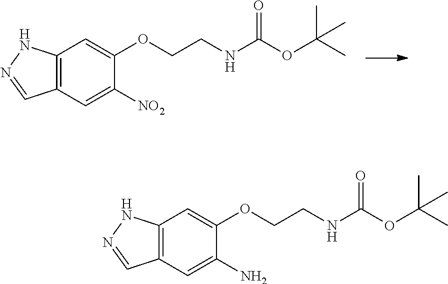 [SMILES: CC(C)(C)OC(=O)NCCOc1cc2[nH]ncc2cc1[N+](=O)[O-] ; CC(C)(C)OC(=O)NCCOc1cc2[nH]ncc2cc1N]

3.43 g (10.6 mmol) tert-butyl {2-[(5-nitro-1H-indazol-6-yl)oxy]ethyl}carbamate (prepared according to intermediate example 130b) were transformed in analogy to intermediate example 94b to give after working up and purification 1.77 g (57%) of the title compound.

### Example 130b

### tert-Butyl {2-[(5-nitro-1H-indazol-6-yl)oxy]ethyl}carbamate

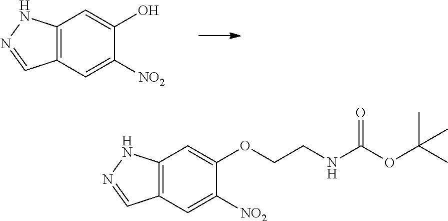 [SMILES: O=[N+]([O-])c1cc2cn[nH]c2cc1O ; CC(C)(C)OC(=O)NCCOc1cc2[nH]ncc2cc1[N+](=O)[O-]]

500 mg (2.79 mmol) 5-nitro-1H-indazol-6-ol (prepared according to intermediate example 94d) were transformed in analogy to intermediate example 94c using tert-butyl (2-hydroxyethyl)carbamate (CAS-No: 26690-80-2) to give after working up and purification 650 mg (72%) of the title compound.

### Example 131

### [(7S)-4-{[6-(2-Aminoethoxy)-1H-indazol-5-yl]amino}-5,6,7,8-tetrahydro[1]benzothieno[2,3-d]pyrimidin-7-yl][(3S)-3-methylmorpholin-4-yl]methanone

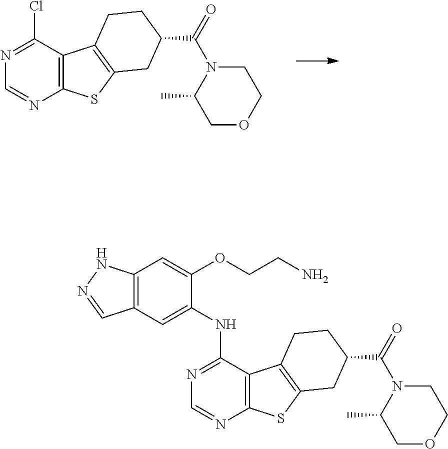 [SMILES: C[C@H]1COCCN1C(=O)[C@H]1CCc2c(sc3ncnc(Cl)c23)C1 ; C[C@H]1COCCN1C(=O)[C@H]1CCc2c(sc3ncnc(Nc4cc5cn[nH]c5cc4OCCN)c23)C1]

50 mg (142 μmol) [(7S)-4-chloro-5,6,7,8-tetrahydro[1]benzothieno[2,3-d]pyrimidin-7-yl][(3S)-3-methylmorpholin-4-yl]methanone (prepared according to intermediate example 117a) were transformed in analogy to intermediate example 130 to give after working up and purification 34.5 mg (45%) of the title compound.

¹H-NMR (DMSO-d6): δ=1.16+1.29 (3H), 1.84 (1H), 2.07 (1H), 2.83-3.90 (14H), 4.08+4.43 (1H), 4.12 (2H), 7.06 (1H), 7.99 (1H), 8.36 (1H), 8.51 (1H), 9.00 (1H), 12.80 (1H) ppm.

### Example 132

### Azetidin-1-yl[(7S)-4-({6-[4-(dimethylamino)piperidin-1-yl]-1H-indazol-5-yl}amino)-5,6,7,8-tetrahydro[1]benzothieno[2,3-d]pyrimidin-7-yl]methanone

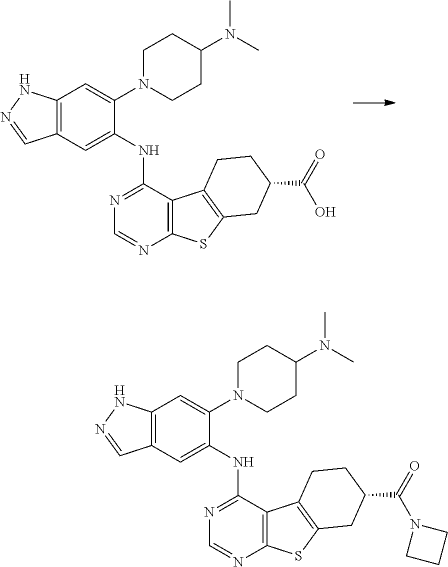 [SMILES: CN(C)C1CCN(c2cc3[nH]ncc3cc2Nc2ncnc3sc4c(c23)CC[C@H](C(=O)O)C4)CC1 ; CN(C)C1CCN(c2cc3[nH]ncc3cc2Nc2ncnc3sc4c(c23)CC[C@H](C(=O)N2CCC2)C4)CC1]

42 mg (85 μmol) (7S)-4-({6-[4-(dimethylamino)piperidin-1-yl]-1H-indazol-5-yl}amino)-5,6,7,8-tetrahydro[1]benzothieno[2,3-d]pyrimidine-7-carboxylic acid (prepared according to intermediate example 132a) were transformed in analogy to example 1 using azetidine to give after working up and purification 7.1 mg (15%) of the title compound.

¹H-NMR (DMSO-d6): δ=1.47-1.67 (2H), 1.76-1.97 (3H), 2.04-2.35 (4H), 2.23 (6H), 2.69-3.43 (9H), 3.88 (2H), 4.12-4.32 (2H), 7.46 (1H), 8.03 (1H), 8.51 (1H), 9.00 (1H), 9.06 (1H), 12.95 (1H) ppm.

### Example 132a

### (7S)-4-({6-[4-(Dimethylamino)piperidin-1-yl]-1H-indazol-5-yl}amino)-5,6,7,8-tetrahydro[1]benzothieno[2,3-d]pyrimidine-7-carboxylic acid

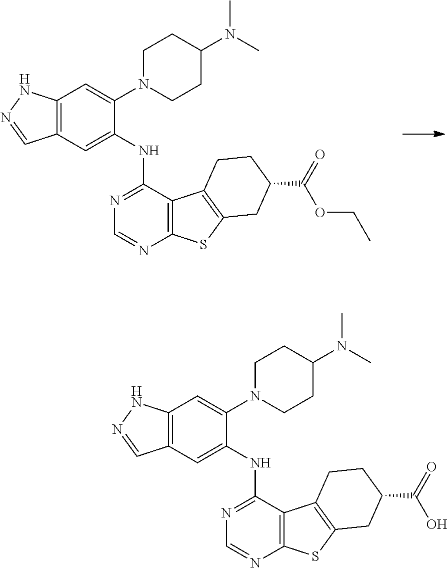 [SMILES: CCOC(=O)[C@H]1CCc2c(sc3ncnc(Nc4cc5cn[nH]c5cc4N4CCC(N(C)C)CC4)c23)C1 ; CN(C)C1CCN(c2cc3[nH]ncc3cc2Nc2ncnc3sc4c(c23)CC[C@H](C(=O)O)C4)CC1]

501 mg (964 μmol) ethyl (7S)-4-({6-[4-(dimethylamino)piperidin-1-yl]-1H-indazol-5-yl}amino)-5,6,7,8-tetrahydro[1]benzothieno[2,3-d]pyrimidine-7-carboxylate (prepared according to intermediate example 132b) were transformed in analogy to intermediate example 1a to give after working up and purification 433 mg (91%) of the title compound.

### Example 132b

### Ethyl (7S)-4-({6-[4-(dimethylamino)piperidin-1-yl]-1H-indazol-5-yl}amino)-5,6,7,8-tetrahydro[1]benzothieno[2,3-d]pyrimidine-7-carboxylate

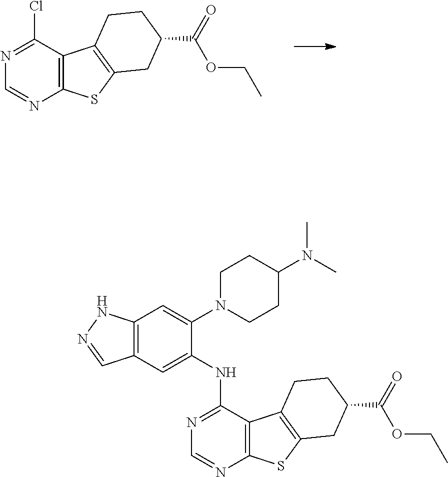 [SMILES: CCOC(=O)[C@H]1CCc2c(sc3ncnc(Cl)c23)C1 ; CCOC(=O)[C@H]1CCc2c(sc3ncnc(Nc4cc5cn[nH]c5cc4N4CCC(N(C)C)CC4)c23)C1]

1.00 g (3.37 mmol) ethyl (7S)-4-chloro-5,6,7,8-tetrahydro[1]benzothieno[2,3-d]pyrimidine-7-carboxylate (prepared according to intermediate example 1c) were transformed in analogy to intermediate example 1b using 6-[4-(dimethylamino)piperidin-1-yl]-1H-indazol-5-amine (prepared according to intermediate example 104a) to give after working up and purification 506 mg (29%) of the title compound.

### Example 133

### [(7S)-4-({6-[4-(Dimethylamino)piperidin-1-yl]-1H-indazol-5-yl}amino)-5,6,7,8-tetrahydro[1]benzothieno[2,3-d]pyrimidin-7-yl](morpholin-4-yl)methanone

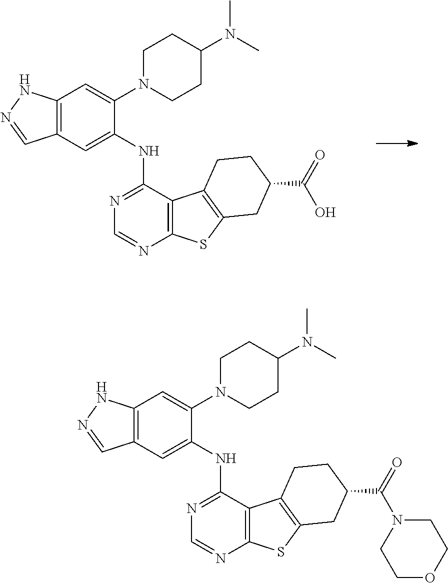 [SMILES: CN(C)C1CCN(c2cc3[nH]ncc3cc2Nc2ncnc3sc4c(c23)CC[C@H](C(=O)O)C4)CC1 ; CN(C)C1CCN(c2cc3[nH]ncc3cc2Nc2ncnc3sc4c(c23)CC[C@H](C(=O)N2CCOCC2)C4)CC1]

42 mg (85 μmol) (7S)-4-({6-[4-(dimethylamino)piperidin-1-yl]-1H-indazol-5-yl}amino)-5,6,7,8-tetrahydro[1]benzothieno[2,3-d]pyrimidine-7-carboxylic acid (prepared according to intermediate example 132a) were transformed in analogy to example 1 using morpholine to give after working up and purification 14.7 mg (29%) of the title compound.

¹H-NMR (DMSO-d6): δ=1.58 (2H), 1.82-1.97 (3H), 2.11 (1H), 2.23 (6H), 2.80 (2H), 2.91 (1H), 2.97-3.09 (3H), 3.20-3.66 (12H), 7.47 (1H), 8.03 (1H), 8.52 (1H), 9.04 (1H), 9.09 (1H), 12.90 (1H) ppm.

### Example 134

### [(7S)-4-({6-[2-(Dimethylamino)ethoxy]-1H-indazol-5-yl}amino)-5,6,7,8-tetrahydro[1]benzothieno[2,3-d]pyrimidin-7-yl][4-(dimethylamino)piperidin-1-yl]methanone

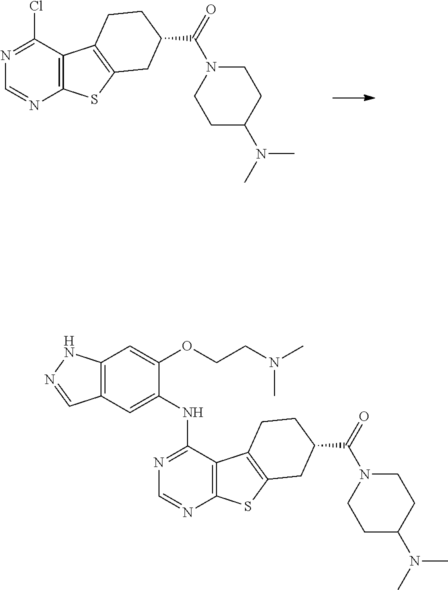 [SMILES: CN(C)C1CCN(C(=O)[C@H]2CCc3c(sc4ncnc(Cl)c34)C2)CC1 ; CN(C)CCOc1cc2[nH]ncc2cc1Nc1ncnc2sc3c(c12)CC[C@H](C(=O)N1CCC(N(C)C)CC1)C3]

50 mg (132 μmol) [(7S)-4-chloro-5,6,7,8-tetrahydro[1]benzothieno[2,3-d]pyrimidin-7-yl][4-(dimethylamino)piperidin-1-yl]methanone (prepared according to intermediate example 134a) were transformed in analogy to intermediate example 1b using 6-[2-(dimethylamino)ethoxy]-1H-indazol-5-amine (prepared according to intermediate example 109a) to give after working up and purification 14.1 mg (18%) of the title compound.

¹H-NMR (DMSO-d6): δ=1.20-1.50 (2H), 1.89 (3H), 2.10 (1H), 2.25 (6H), 2.28-3.43 (16H), 4.12 (1H), 4.27 (2H), 4.46 (1H), 7.15 (1H), 8.00 (1H), 8.26 (1H), 8.51 (1H), 8.95 (1H), 12.84 (1H) ppm.

### Example 134a

### [(7S)-4-Chloro-5,6,7,8-tetrahydro[1]benzothieno[2,3-d]pyrimidin-7-yl][4-(dimethylamino)piperidin-1-yl]methanone

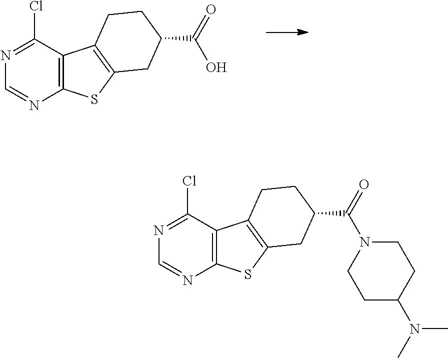 [SMILES: O=C(O)[C@H]1CCc2c(sc3ncnc(Cl)c23)C1 ; CN(C)C1CCN(C(=O)[C@H]2CCc3c(sc4ncnc(Cl)c34)C2)CC1]

500 mg (1.86 mmol) (7S)-4-chloro-5,6,7,8-tetrahydro[1]benzothieno[2,3-d]pyrimidine-7-carboxylic acid (prepared according to intermediate example 66b) were transformed in analogy to example 1 using N,N-dimethylpiperidin-4-amine to give after working up and purification 503 mg (71%) of the title compound.

### Example 135

### [(7S)-4-({6-[4-(Dimethylamino)piperidin-1-yl]-1H-indazol-5-yl}amino)-5,6,7,8-tetrahydro[1]benzothieno[2,3-d]pyrimidin-7-yl][(3S)-3-methylmorpholin-4-yl]methanone

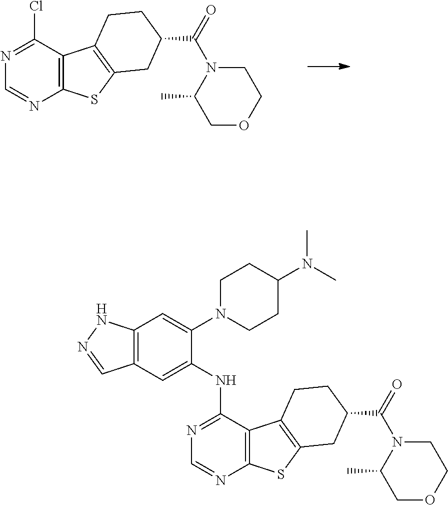 [SMILES: C[C@H]1COCCN1C(=O)[C@H]1CCc2c(sc3ncnc(Cl)c23)C1 ; C[C@H]1COCCN1C(=O)[C@H]1CCc2c(sc3ncnc(Nc4cc5cn[nH]c5cc4N4CCC(N(C)C)CC4)c23)C1]

50 mg (142 μmol) [(7S)-4-chloro-5,6,7,8-tetrahydro[1]benzothieno[2,3-d]pyrimidin-7-yl][(3S)-3-methylmorpholin-4-yl]methanone (prepared according to intermediate example 117a) were transformed in analogy to intermediate example 1b using 6-[4-(dimethylamino)piperidin-1-yl]-1H-indazol-5-amine (prepared according to intermediate example 104a) to give after working up and purification 24.0 mg (28%) of the title compound.

¹H-NMR (DMSO-d6): δ=1.14+1.29 (3H), 1.46-2.00 (5H), 2.10 (1H), 2.23 (6H), 2.18-2.37 (1H), 2.69-4.47 (16H), 7.48 (1H), 8.03 (1H), 8.53 (1H), 9.06 (1H), 9.10+9.15 (1H), 12.92 (1H) ppm.

### Example 136

### [(7S)-4-({6-[4-(Dimethylamino)piperidin-1-yl]-1H-indazol-5-yl}amino)-5,6,7,8-tetrahydro[1]benzothieno[2,3-d]pyrimidin-7-yl][(3R)-3-methylmorpholin-4-yl]methanone

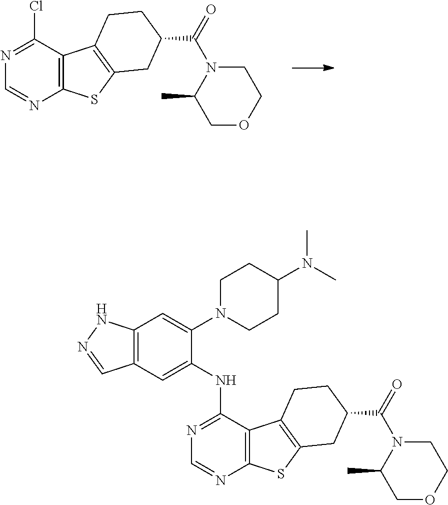 [SMILES: C[C@@H]1COCCN1C(=O)[C@H]1CCc2c(sc3ncnc(Cl)c23)C1 ; C[C@@H]1COCCN1C(=O)[C@H]1CCc2c(sc3ncnc(Nc4cc5cn[nH]c5cc4N4CCC(N(C)C)CC4)c23)C1]

50 mg (142 μmol) [(7S)-4-chloro-5,6,7,8-tetrahydro[1]benzothieno[2,3-d]pyrimidin-7-yl][(3R)-3-methylmorpholin-4-yl]methanone (prepared according to intermediate example 114a) were transformed in analogy to intermediate example 1b using 6-[4-(dimethylamino)piperidin-1-yl]-1H-indazol-5-amine (prepared according to intermediate example 104a) to give after working up and purification 18.8 mg (22%) of the title compound.

¹H-NMR (DMSO-d6): δ=1.15+1.28 (3H), 1.62 (1H), 1.58 (2H), 1.90 (3H), 2.07 (1H), 2.16-2.36 (2H), 2.22 (6H), 2.69-4.44 (14H), 7.47 (1H), 8.03 (1H), 8.52 (1H), 9.03 (1H), 9.08 (1H), 12.91 (1H) ppm.

### Example 137

### [(7S)-4-({6-[4-(Dimethylamino)piperidin-1-yl]-1H-indazol-5-yl}amino)-5,6,7,8-tetrahydro[1]benzothieno[2,3-d]pyrimidin-7-yl][(2R,6S)-2,6-dimethylmorpholin-4-yl]methanone

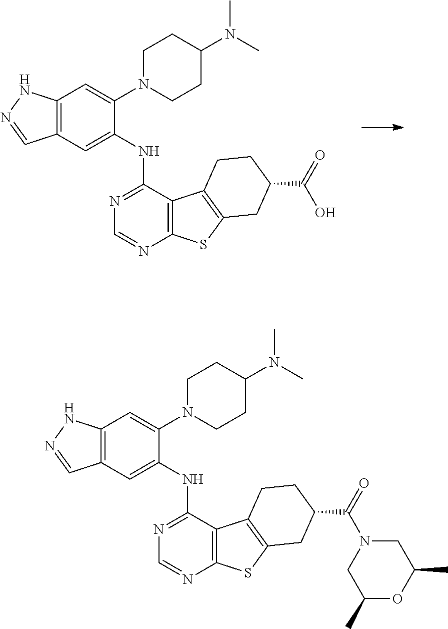 [SMILES: CN(C)C1CCN(c2cc3[nH]ncc3cc2Nc2ncnc3sc4c(c23)CC[C@H](C(=O)O)C4)CC1 ; C[C@@H]1CN(C(=O)[C@H]2CCc3c(sc4ncnc(Nc5cc6cn[nH]c6cc5N5CCC(N(C)C)CC5)c34)C2)C[C@H](C)O1]

42 mg (85 μmol) (7S)-4-({6-[4-(dimethylamino)piperidin-1-yl]-1H-indazol-5-yl}amino)-5,6,7,8-tetrahydro[1]benzothieno[2,3-d]pyrimidine-7-carboxylic acid (prepared according to intermediate example 132a) were transformed in analogy to example 1 using (2R,6S)-2,6-dimethylmorpholine to give after working up and purification 19.8 mg (37%) of the title compound.

¹H-NMR (DMSO-d6): δ=1.11 (6H), 1.50-1.68 (2H), 1.91 (2H), 1.83-1.98 (3H), 2.09 (1H), 2.23 (6H), 2.27 (1H), 2.68-3.10 (6H), 3.21-3.56 (3H), 3.96 (1H), 4.30 (1H), 7.48 (1H), 8.03 (1H), 8.52 (1H), 9.05 (1H), 9.10 (1H), 12.90 (1H) ppm.

### Example 138

### [4-(Dimethylamino)piperidin-1-yl][(7S)-4-({6-[2-(pyrrolidin-1-yl)ethoxy]-1H-indazol-5-yl}amino)-5,6,7,8-tetrahydro[1]benzothieno[2,3-d]pyrimidin-7-yl]methanone

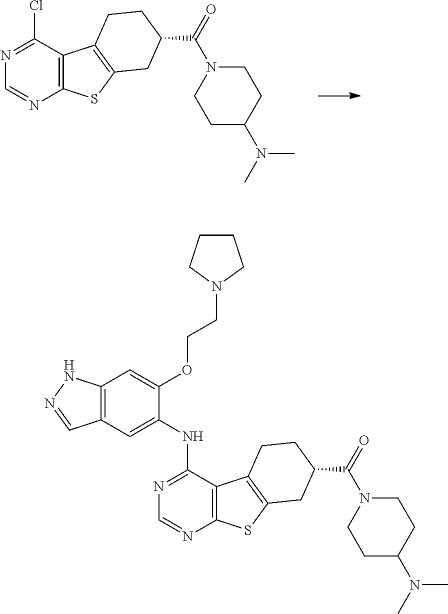 [SMILES: CN(C)C1CCN(C(=O)[C@H]2CCc3c(sc4ncnc(Cl)c34)C2)CC1 ; CN(C)C1CCN(C(=O)[C@H]2CCc3c(sc4ncnc(Nc5cc6cn[nH]c6cc5OCCN5CCCC5)c34)C2)CC1]

50 mg (132 μmol) [(7S)-4-chloro-5,6,7,8-tetrahydro[1]benzothieno[2,3-d]pyrimidin-7-yl][4-(dimethylamino)piperidin-1-yl]methanone (prepared according to intermediate example 134a) were transformed in analogy to intermediate example 1b using 6-[2-(pyrrolidin-1-yl)ethoxy]-1H-indazol-5-amine (prepared according to intermediate example 112a) to give after working up and purification 24.1 mg (29%) of the title compound.

¹H-NMR (DMSO-d6): δ=1.13-1.41 (2H), 1.66 (4H), 1.71-1.91 (3H), 2.08 (1H), 2.17 (6H), 2.33 (1H), 2.49 (4H), 2.61 (1H), 2.82-3.26 (8H), 4.05 (1H), 4.27 (2H), 4.40 (1H), 7.14 (1H), 7.99 (1H), 8.29 (1H), 8.50 (1H), 8.93+8.98 (1H), 12.83 (1H) ppm.

### Example 139

[4-(Dimethylamino)piperidin-1-yl][(7S)-4-({6-[2-(piperidin-1-yl)ethoxy]-1H-indazol-5-yl}amino)-5,6,7,8-tetrahydro[1]benzothieno[2,3-d]pyrimidin-7-yl]methanone

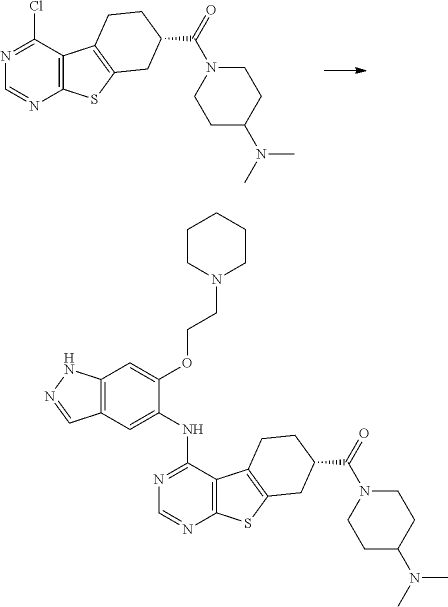

50 mg (132 μmol) [(7S)-4-chloro-5,6,7,8-tetrahydro[1]benzothieno[2,3-d]pyrimidin-7-yl][4-(dimethylamino)piperidin-1-yl]methanone (prepared according to intermediate example 134a) were transformed in analogy to intermediate example 1b using 6-[2-(piperidin-1-yl)ethoxy]-1H-indazol-5-amine (prepared according to intermediate example 113a) to give after working up and purification 24.3 mg (29%) of the title compound.

¹H-NMR (DMSO-d6): δ=1.20 (1H), 1.28-1.50 (7H), 1.71-1.92 (3H), 2.09 (1H), 2.17 (6H), 2.40 (4H), 2.61 (1H), 2.76 (2H), 2.90 (1H), 2.96-3.26 (6H), 4.05 (1H), 4.27 (2H), 4.40 (1H), 7.15 (1H), 7.99 (1H), 8.27 (1H), 8.50 (1H), 8.92+8.97 (1H), 12.84 (1H) ppm.

### Example 140

### {(7S)-4-[(6-{[(2R)-1-Aminopropan-2-yl]oxy}-1H-indazol-5-yl)amino]-5,6,7,8-tetrahydro[1]benzothieno[2,3-d]pyrimidin-7-yl}[(3S)-3-methylmorpholin-4-yl]methanone

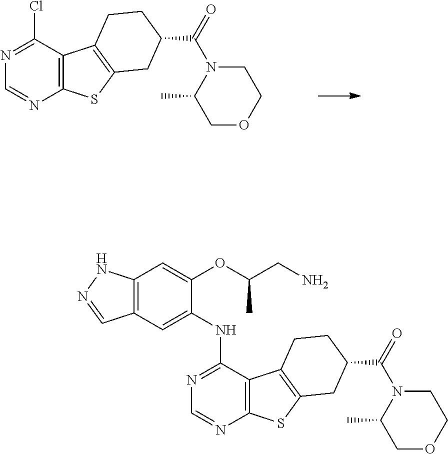 [SMILES: C[C@H]1COCCN1C(=O)[C@H]1CCc2c(sc3ncnc(Cl)c23)C1 ; C[C@H](CN)Oc1cc2[nH]ncc2cc1Nc1ncnc2sc3c(c12)CC[C@H](C(=O)N1CCOC[C@@H]1C)C3]

50 mg (142 μmol) [(7S)-4-chloro-5,6,7,8-tetrahydro[1]benzothieno[2,3-d]pyrimidin-7-yl][(3S)-3-methylmorpholin-4-yl]methanone (prepared according to intermediate example 117a) were transformed in analogy to intermediate example 130 using tert-butyl {(2R)-2-[(5-amino-1H-indazol-6-yl)oxy]propyl}carbamate (prepared according to intermediate example 140a) to give after working up and purification 20.5 mg (49%) of the title compound.

¹H-NMR (DMSO-d6): δ=1.16+1.30 (3H), 1.36 (3H), 1.86 (1H), 2.12 (1H), 2.85-4.49 (14H), 4.98 (1H), 7.20 (1H), 7.91 (2H), 8.02 (1H), 8.26 (1H), 8.52 (1H), 9.03 (1H), 12.86 (1H) ppm.

### Example 140a

### tert-Butyl {(2R)-2-[(5-amino-1H-indazol-6-yl)oxy]propyl}carbamate

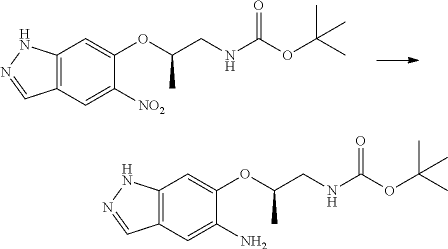 [SMILES: C[C@H](CNC(=O)OC(C)(C)C)Oc1cc2[nH]ncc2cc1[N+](=O)[O-] ; C[C@H](CNC(=O)OC(C)(C)C)Oc1cc2[nH]ncc2cc1N]

808 mg (2.40 mmol) tert-butyl {(2R)-2-[(5-nitro-1H-indazol-6-yl)oxy]propyl}carbamate (prepared according to intermediate example 140b) were transformed in analogy to intermediate example 94b to give after working up and purification 353 mg (48%) of the title compound.

### Example 140b

### tert-Butyl {(2R)-2-[(5-nitro-1H-indazol-6-yl)oxy]propyl}carbamate

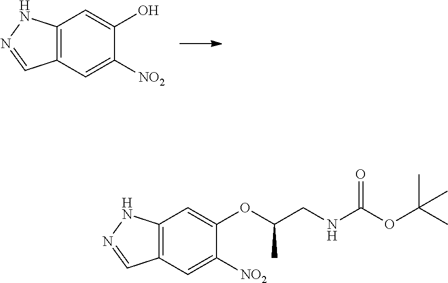 [SMILES: O=[N+]([O-])c1cc2cn[nH]c2cc1O ; C[C@H](CNC(=O)OC(C)(C)C)Oc1cc2[nH]ncc2cc1[N+](=O)[O-]]

500 mg (2.79 mmol) 5-nitro-1H-indazol-6-ol (prepared according to intermediate example 94d) were transformed in analogy to intermediate example 94c using tert-butyl [(2R)-2-hydroxypropyl]carbamate (CAS-No: 119768-44-4) to give after working up and purification 813 mg (87%) of the title compound.

### Example 141

### (7S)-4-[(6-{[(25)-1-Aminopropan-2-yl]oxy}-1H-indazol-5-yl)amino]-N,N-dimethyl-5,6,7,8-tetrahydro[1]benzothieno[2,3-d]pyrimidine-7-carboxamide

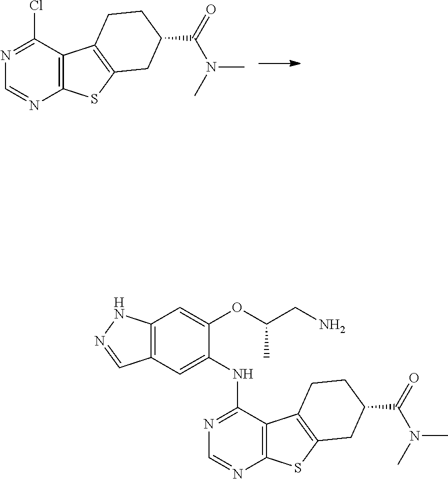 [SMILES: CN(C)C(=O)[C@H]1CCc2c(sc3ncnc(Cl)c23)C1 ; C[C@@H](CN)Oc1cc2[nH]ncc2cc1Nc1ncnc2sc3c(c12)CC[C@H](C(=O)N(C)C)C3]

50 mg (169 μmol) (7S)-4-chloro-N,N-dimethyl-5,6,7,8-tetrahydro[1]benzothieno[2,3-d]pyrimidine-7-carboxamide (prepared according to intermediate example 94a) were transformed in analogy to intermediate example 130 using tert-butyl {(2S)-2-[(5-amino-1H-indazol-6-yl)oxy]propyl}carbamate (prepared according to intermediate example 141a) to give after working up and purification 55.1 mg (67%) of the title compound.

¹H-NMR (DMSO-d6): δ=1.33 (3H), 1.85 (1H), 2.10 (1H), 2.79-2.97 (4H), 2.87 (3H), 3.10 (3H), 3.12-3.26 (2H), 4.64 (1H), 7.13 (1H), 7.99 (1H), 8.38 (1H), 8.51 (1H), 9.03 (1H), 12.79 (1H) ppm.

### Example 141a

### tert-Butyl {(2S)-2-[(5-amino-1H-indazol-6-yl)oxy]propyl}carbamate

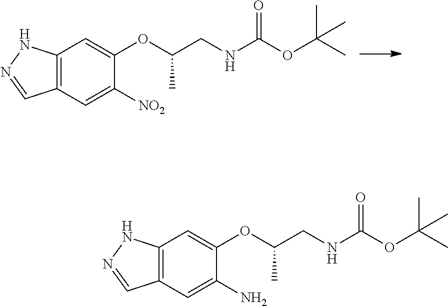 [SMILES: C[C@@H](CNC(=O)OC(C)(C)C)Oc1cc2[nH]ncc2cc1[N+](=O)[O-] ; C[C@@H](CNC(=O)OC(C)(C)C)Oc1cc2[nH]ncc2cc1N]

1.03 g (3.05 mmol) tert-butyl {(2S)-2-[(5-nitro-1H-indazol-6-yl)oxy]propyl}carbamate (prepared according to intermediate example 141b) were transformed in analogy to intermediate example 94b to give after working up and purification 466 mg (50%) of the title compound.

### Example 141b

### tert-Butyl {(2R)-2-[(5-nitro-1H-indazol-6-yl)oxy]propyl}carbamate

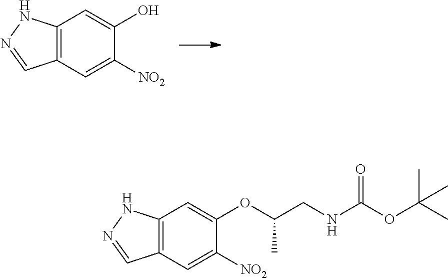 [SMILES: O=[N+]([O-])c1cc2cn[nH]c2cc1O ; C[C@@H](CNC(=O)OC(C)(C)C)Oc1cc2[nH]ncc2cc1[N+](=O)[O-]]

500 mg (2.79 mmol) 5-nitro-1H-indazol-6-ol (prepared according to intermediate example 94d) were transformed in analogy to intermediate example 94c using tert-butyl [(2S)-2-hydroxypropyl]carbamate (CAS-No: 167938-56-9) to give after working up and purification 938 mg (100%) of the title compound.

### Example 142

### (7S)-4-[(6-{[(2S)-2-Aminopropyl]oxy}-1H-indazol-5-yl)amino]-N,N-dimethyl-5,6,7,8-tetrahydro[1]benzothieno[2,3-d]pyrimidine-7-carboxamide

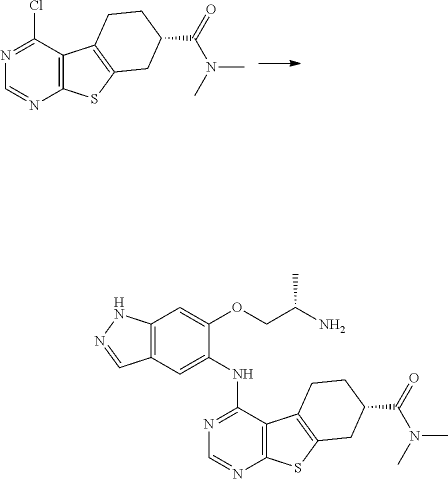 [SMILES: CN(C)C(=O)[C@H]1CCc2c(sc3ncnc(Cl)c23)C1 ; C[C@H](N)COc1cc2[nH]ncc2cc1Nc1ncnc2sc3c(c12)CC[C@H](C(=O)N(C)C)C3]

50 mg (169 μmol) (7S)-4-chloro-N,N-dimethyl-5,6,7,8-tetrahydro[1]benzothieno[2,3-d]pyrimidine-7-carboxamide (prepared according to intermediate example 94a) were transformed in analogy to intermediate example 130 using tert-butyl {(2S)-1-[(5-amino-1H-indazol-6-yl)oxy]propan-2-yl}carbamate (prepared according to intermediate example 142a) to give after working up and purification 59.3 mg (72%) of the title compound.

¹H-NMR (DMSO-d6): δ=1.11 (3H), 1.82 (1H), 2.10 (1H), 2.88 (3H), 2.92 (2H), 3.05-3.42 (6H), 3.10 (3H), 3.91 (2H), 7.05 (1H), 7.99 (1H), 8.30 (1H), 8.49 (1H), 8.96 (1H), 12.81 (1H) ppm.

### Example 142a

### tert-Butyl {(2S)-1-[(5-amino-1H-indazol-6-yl)oxy]propan-2-yl}carbamate

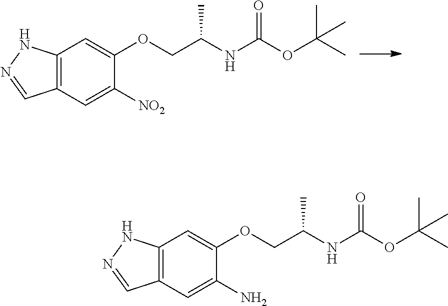 [SMILES: C[C@@H](COc1cc2[nH]ncc2cc1[N+](=O)[O-])NC(=O)OC(C)(C)C ; C[C@@H](COc1cc2[nH]ncc2cc1N)NC(=O)OC(C)(C)C]

960 mg (2.85 mmol) tert-butyl {(2S)-1-[(5-nitro-1H-indazol-6-yl)oxy]propan-2-yl}carbamate (prepared according to intermediate example 142b) were transformed in analogy to intermediate example 94b to give after working up and purification 382 mg (44%) of the title compound.

### Example 142b

### tert-Butyl {(2S)-1-[(5-nitro-1H-indazol-6-yl)oxy]propan-2-yl}carbamate

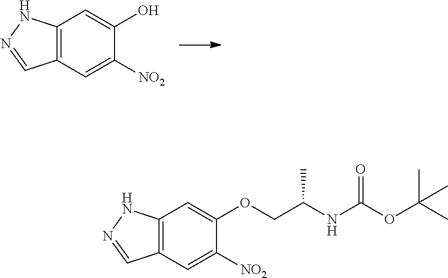 [SMILES: O=[N+]([O-])c1cc2cn[nH]c2cc1O ; C[C@@H](COc1cc2[nH]ncc2cc1[N+](=O)[O-])NC(=O)OC(C)(C)C]

500 mg (2.79 mmol) 5-nitro-1H-indazol-6-ol (prepared according to intermediate example 94d) were transformed in analogy to intermediate example 94c using tert-butyl [(2S)-1-hydroxypropan-2-yl]carbamate (CAS-No: 79069-13-9) to give after working up and purification 935 mg (100%) of the title compound.

### Example 143

### {(7S)-4-[(6-{[(2S)-2-Aminopropyl]oxy}-1H-indazol-5-yl)amino]-5,6,7,8-tetrahydro[1]benzothieno[2,3-d]pyrimidin-7-yl}[(3S)-3-methylmorpholin-4-yl]methanone

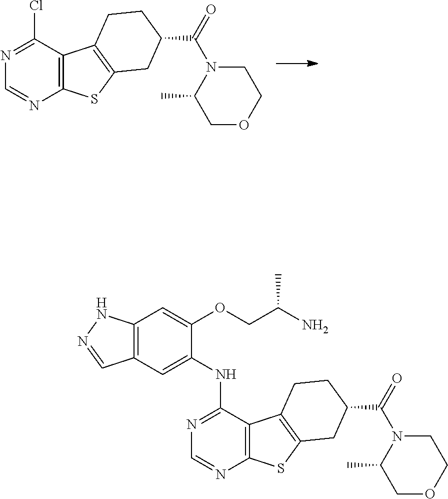 [SMILES: C[C@H]1COCCN1C(=O)[C@H]1CCc2c(sc3ncnc(Cl)c23)C1 ; C[C@H](N)COc1cc2[nH]ncc2cc1Nc1ncnc2sc3c(c12)CC[C@H](C(=O)N1CCOC[C@@H]1C)C3]

50 mg (142 μmol) [(7S)-4-chloro-5,6,7,8-tetrahydro[1]benzothieno[2,3-d]pyrimidin-7-yl][(3S)-3-methylmorpholin-4-yl]methanone (prepared according to intermediate example 117a) were transformed in analogy to intermediate example 130 using tert-butyl {(2S)-1-[(5-amino-1H-indazol-6-yl)oxy]propan-2-yl}carbamate (prepared according to intermediate example 142a) to give after working up and purification 45.8 mg (59%) of the title compound.

¹H-NMR (DMSO-d6): δ=1.11 (3H), 1.16+1.29 (3H), 1.63-1.95 (1H), 2.08 (1H), 2.81-4.48 (17H), 7.05 (1H), 7.98 (1H), 8.29 (1H), 8.49 (1H), 8.94 (1H), 12.81 (1H) ppm.

### Example 144

### {(7S)-4-[(6-{[(2R)-1-Aminopropan-2-yl]oxy}-1H-indazol-5-yl)amino]-5,6,7,8-tetrahydro[1]benzothieno[2,3-d]pyrimidin-7-yl}[(3R)-3-methylmorpholin-4-yl]methanone

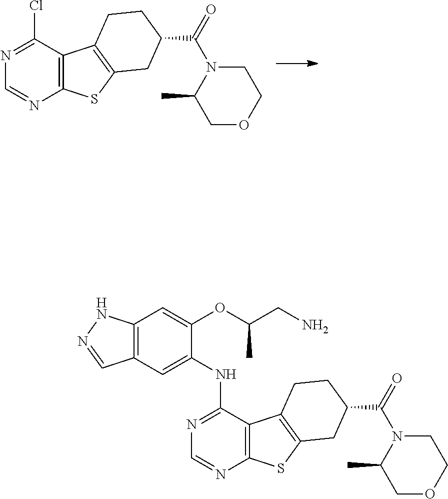 [SMILES: C[C@@H]1COCCN1C(=O)[C@H]1CCc2c(sc3ncnc(Cl)c23)C1 ; C[C@H](CN)Oc1cc2[nH]ncc2cc1Nc1ncnc2sc3c(c12)CC[C@H](C(=O)N1CCOC[C@H]1C)C3]

50 mg (142 μmol) [(7S)-4-chloro-5,6,7,8-tetrahydro[1]benzothieno[2,3-d]pyrimidin-7-yl][(3R)-3-methylmorpholin-4-yl]methanone (prepared according to intermediate example 114a) were transformed in analogy to intermediate example 130 using tert-butyl {(2R)-2-[(5-amino-1H-indazol-6-yl)oxy]propyl}carbamate (prepared according to intermediate example 140a) to give after working up and purification 44.4 mg (57%) of the title compound.

¹H-NMR (DMSO-d6): δ=1.15+1.31 (3H), 1.37 (3H), 1.93 (1H), 2.13 (1H), 2.79-4.47 (14H), 5.03 (1H), 7.21 (1H), 8.02 (1H), 8.21 (2H), 8.28 (1H), 8.52 (1H), 9.03 (1H), 12.90 (1H) ppm.

### Example 145

### {(7S)-4-[(6-{[(2S)-1-Aminopropan-2-yl]oxy}-1H-indazol-5-yl)amino]-5,6,7,8-tetrahydro[1]benzothieno[2,3-d]pyrimidin-7-yl}[(3S)-3-methylmorpholin-4-yl]methanone

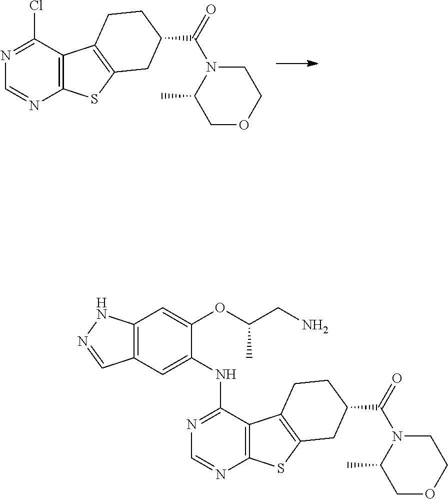 [SMILES: C[C@H]1COCCN1C(=O)[C@H]1CCc2c(sc3ncnc(Cl)c23)C1 ; C[C@@H](CN)Oc1cc2[nH]ncc2cc1Nc1ncnc2sc3c(c12)CC[C@H](C(=O)N1CCOC[C@@H]1C)C3]

50 mg (142 μmol) [(7S)-4-chloro-5,6,7,8-tetrahydro[1]benzothieno[2,3-d]pyrimidin-7-yl][(3S)-3-methylmorpholin-4-yl]methanone (prepared according to intermediate example 117a) were transformed in analogy to intermediate example 130 using tert-butyl {(2S)-2-[(5-amino-1H-indazol-6-yl)oxy]propyl}carbamate (prepared according to intermediate example 141a) to give after working up and purification 51.6 mg (66%) of the title compound.

¹H-NMR (DMSO-d6): δ=1.17+1.31 (3H), 1.38 (3H), 1.87 (1H), 2.11 (1H), 2.87-4.51 (14H), 5.01 (1H), 7.20 (1H), 8.02 (1H), 8.22 (1H), 8.26 (2H), 8.51 (1H), 9.00 (1H), 12.87 (1H) ppm.

### Example 146

### {(7S)-4-[(6-{[(2S)-2-Aminopropyl]oxy}-1H-indazol-5-yl)amino]-5,6,7,8-tetrahydro[1]benzothieno[2,3-d]pyrimidin-7-yl}[(3R)-3-methylmorpholin-4-yl]methanone

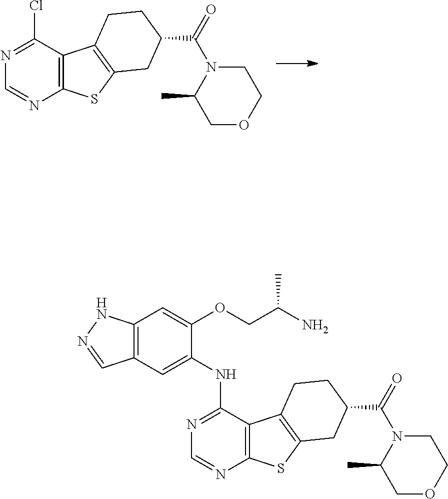 [SMILES: C[C@@H]1COCCN1C(=O)[C@H]1CCc2c(sc3ncnc(Cl)c23)C1 ; C[C@H](N)COc1cc2[nH]ncc2cc1Nc1ncnc2sc3c(c12)CC[C@H](C(=O)N1CCOC[C@H]1C)C3]

50 mg (142 μmol) [(7S)-4-chloro-5,6,7,8-tetrahydro[1]benzothieno[2,3-d]pyrimidin-7-yl][(3R)-3-methylmorpholin-4-yl]methanone (prepared according to intermediate example 114a) were transformed in analogy to intermediate example 130 using tert-butyl {(2S)-1-[(5-amino-1H-indazol-6-yl)oxy]propan-2-yl}carbamate (prepared according to intermediate example 142a) to give after working up and purification 37.3 mg (48%) of the title compound.

¹H-NMR (DMSO-d6): δ=1.11 (3H), 1.15+1.33 (3H), 1.92 (1H), 2.07 (1H), 2.82-4.48 (17H), 7.05 (1H), 7.99 (1H), 8.31 (1H), 8.49 (1H), 8.96 (1H), 12.81 (1H) ppm.

### Example 147

### (7S)-4-[(6-{[(2R)-2-Aminopropyl]oxy}-1H-indazol-5-yl)amino]-N,N-dimethyl-5,6,7,8-tetrahydro[1]benzothieno[2,3-d]pyrimidine-7-carboxamide

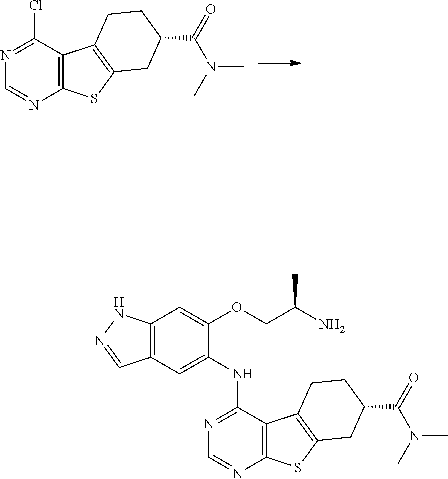 [SMILES: CN(C)C(=O)[C@H]1CCc2c(sc3ncnc(Cl)c23)C1 ; C[C@@H](N)COc1cc2[nH]ncc2cc1Nc1ncnc2sc3c(c12)CC[C@H](C(=O)N(C)C)C3]

50 mg (169 μmol) (7S)-4-chloro-N,N-dimethyl-5,6,7,8-tetrahydro[1]benzothieno[2,3-d]pyrimidine-7-carboxamide (prepared according to intermediate example 94a) were transformed in analogy to intermediate example 130 using tert-butyl {(2R)-1-[(5-amino-1H-indazol-6-yl)oxy]propan-2-yl}carbamate (prepared according to intermediate example 147a) to give after working up and purification 51.6 mg (62%) of the title compound.

¹H-NMR (DMSO-d6): δ=1.10 (3H), 1.68-1.91 (2H), 2.10 (1H), 2.87 (3H), 2.91 (2H), 3.06-3.28 (5H), 3.10 (3H), 3.90 (2H), 7.04 (1H), 7.98 (1H), 8.29 (1H), 8.49 (1H), 8.97 (1H), 12.82 (1H) ppm.

### Example 147a

### tert-Butyl {(2R)-1-[(5-amino-1H-indazol-6-yl)oxy]propan-2-yl}carbamate

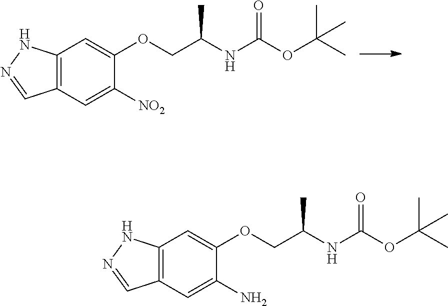 [SMILES: C[C@H](COc1cc2[nH]ncc2cc1[N+](=O)[O-])NC(=O)OC(C)(C)C ; C[C@H](COc1cc2[nH]ncc2cc1N)NC(=O)OC(C)(C)C]

894 mg (2.66 mmol) tert-butyl {(2R)-1-[(5-nitro-1H-indazol-6-yl)oxy]propan-2-yl}carbamate (prepared according to intermediate example 147b) were transformed in analogy to intermediate example 94b to give after working up and purification 425 mg (52%) of the title compound.

### Example 147b

### tert-Butyl {(2R)-1-[(5-nitro-1H-indazol-6-yl)oxy]propan-2-yl}carbamate

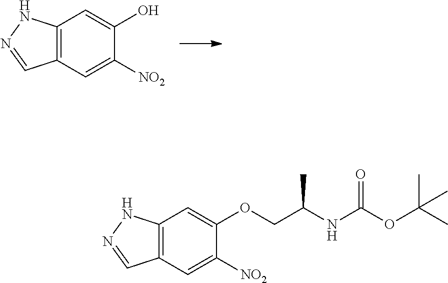 [SMILES: O=[N+]([O-])c1cc2cn[nH]c2cc1O ; C[C@H](COc1cc2[nH]ncc2cc1[N+](=O)[O-])NC(=O)OC(C)(C)C]

500 mg (2.79 mmol) 5-nitro-1H-indazol-6-ol (prepared according to intermediate example 94d) were transformed in analogy to intermediate example 94c using tert-butyl [(2R)-1-hydroxypropan-2-yl]carbamate (CAS-No: 106391-86-0) to give after working up and purification 899 mg (96%) of the title compound.

### Example 148

### {(7S)-4-[(6-{[(2S)-1-Aminopropan-2-yl]oxy}-1H-indazol-5-yl)amino]-5,6,7,8-tetrahydro[1]benzothieno[2,3-d]pyrimidin-7-yl}[(3R)-3-methylmorpholin-4-yl]methanone

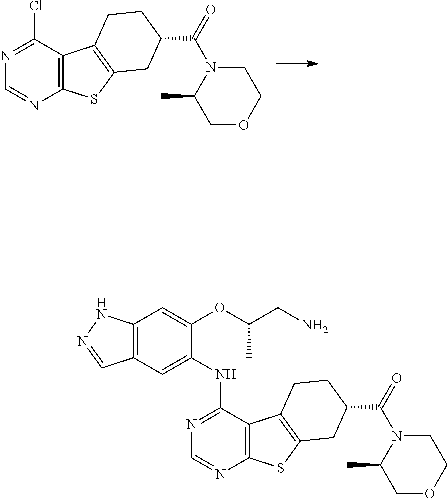 [SMILES: C[C@@H]1COCCN1C(=O)[C@H]1CCc2c(sc3ncnc(Cl)c23)C1 ; C[C@@H]1COCCN1C(=O)[C@H]1CCc2c(sc3ncnc(Nc4cc5cn[nH]c5cc4O[C@@H](C)CN)c23)C1]

50 mg (142 μmol) [(7S)-4-chloro-5,6,7,8-tetrahydro[1]benzothieno[2,3-d]pyrimidin-7-yl][(3R)-3-methylmorpholin-4-yl]methanone (prepared according to intermediate example 114a) were transformed in analogy to intermediate example 130 using tert-butyl {(2S)-2-[(5-amino-1H-indazol-6-yl)oxy]propyl}carbamate (prepared according to intermediate example 141a) to give after working up and purification 37.3 mg (48%) of the title compound.

¹H-NMR (DMSO-d6): δ=1.15+1.33 (6H), 1.95 (1H), 2.09 (1H), 2.78-4.47 (16H), 4.66 (1H), 7.14 (1H), 7.99 (1H), 8.38 (1H), 8.51 (1H), 9.03 (1H), 12.78 (1H) ppm.

### Example 149

### {(7S)-4-[(6-{[(2R)-2-Aminopropyl]oxy}-1H-indazol-5-yl)amino]-5,6,7,8-tetrahydro[1]benzothieno[2,3-d]pyrimidin-7-yl}[(3S)-3-methylmorpholin-4-yl]methanone

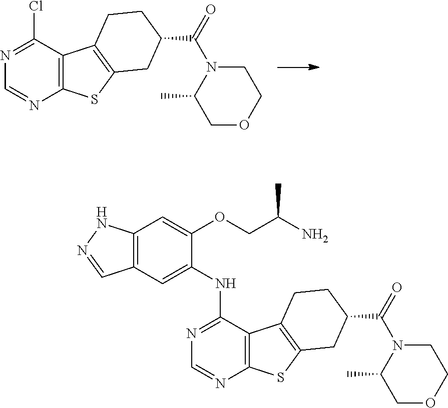 [SMILES: C[C@H]1COCCN1C(=O)[C@H]1CCc2c(sc3ncnc(Cl)c23)C1 ; C[C@@H](N)COc1cc2[nH]ncc2cc1Nc1ncnc2sc3c(c12)CC[C@H](C(=O)N1CCOC[C@@H]1C)C3]

50 mg (142 μmol) [(7S)-4-chloro-5,6,7,8-tetrahydro[1]benzothieno[2,3-d]pyrimidin-7-yl][(3S)-3-methylmorpholin-4-yl]methanone (prepared according to intermediate example 117a) were transformed in analogy to intermediate example 130 using tert-butyl {(2R)-1-[(5-amino-1H-indazol-6-yl)oxy]propan-2-yl}carbamate (prepared according to intermediate example 147a) to give after working up and purification 46.3 mg (59%) of the title compound.

¹H-NMR (DMSO-d6): δ=1.10 (3H), 1.16+1.29 (3H), 1.85 (1H), 2.06 (1H), 2.80-4.48 (17H), 7.04 (1H), 7.98 (1H), 8.28 (1H), 8.48 (1H), 8.95 (1H), 12.82 (1H) ppm.

### Example 150

### {(7S)-4-[(6-{[(2R)-2-Aminopropyl]oxy}-1H-indazol-5-yl)amino]-5,6,7,8-tetrahydro[1]benzothieno[2,3-d]pyrimidin-7-yl}[(3R)-3-methylmorpholin-4-yl]methanone

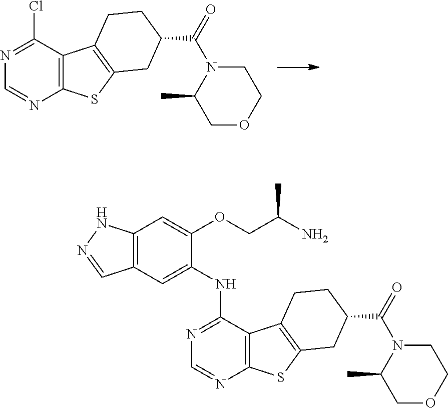 [SMILES: C[C@@H]1COCCN1C(=O)[C@H]1CCc2c(sc3ncnc(Cl)c23)C1 ; C[C@@H]1COCCN1C(=O)[C@H]1CCc2c(sc3ncnc(Nc4cc5cn[nH]c5cc4OC[C@@H](C)N)c23)C1]

50 mg (142 μmol) [(7S)-4-chloro-5,6,7,8-tetrahydro[1]benzothieno[2,3-d]pyrimidin-7-yl][(3R)-3-methylmorpholin-4-yl]methanone (prepared according to intermediate example 114a) were transformed in analogy to intermediate example 130 using tert-butyl {(2R)-1-[(5-amino-1H-indazol-6-yl)oxy]propan-2-yl}carbamate (prepared according to intermediate example 147a) to give after working up and purification 27.1 mg (35%) of the title compound.

¹H-NMR (DMSO-d6): δ=1.11 (3H), 1.15+1.31 (3H), 1.90 (1H), 2.07 (1H), 2.84-4.46 (17H), 7.04 (1H), 7.99 (1H), 8.30 (1H), 8.49 (1H), 8.96 (1H), 12.82 (1H) ppm.

### Example 151

### (7S)-4-[(6-{[2-(Dimethylamino)ethyl](methyl)amino}-1H-indazol-5-yl)amino]-N,N-dimethyl-5,6,7,8-tetrahydro[1]benzothieno[2,3-d]pyrimidine-7-carboxamide

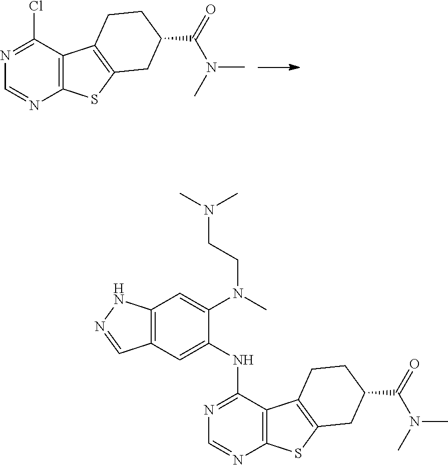 [SMILES: CN(C)C(=O)[C@H]1CCc2c(sc3ncnc(Cl)c23)C1 ; CN(C)CCN(C)c1cc2[nH]ncc2cc1Nc1ncnc2sc3c(c12)CC[C@H](C(=O)N(C)C)C3]

126.8 mg (429 μmol) (7S)-4-chloro-N,N-dimethyl-5,6,7,8-tetrahydro[1]benzothieno[2,3-d]pyrimidine-7-carboxamide (prepared according to intermediate example 94a) were transformed in analogy to intermediate example 1b using N⁶-[2-(dimethylamino)ethyl]-N⁶-methyl-1H-indazole-5,6-diamine (prepared according to intermediate example 151a) to give after working up and purification 9.0 mg (4%) of the title compound.

¹H-NMR (DMSO-d6): δ=1.84 (1H), 1.99 (6H), 2.15 (1H), 2.23 (2H), 2.69 (3H), 2.87 (3H), 2.95 (2H), 3.08 (2H), 3.10 (3H), 3.14-3.41 (3H), 7.49 (1H), 8.04 (1H), 8.51 (1H), 9.01 (1H), 9.44 (1H), 12.88 (1H) ppm.

### Example 151a

### N⁶-[2-(Dimethylamino)ethyl]-N⁶-methyl-1H-indazole-5,6-diamine

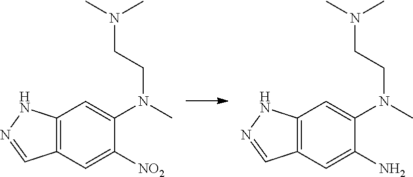 [SMILES: CN(C)CCN(C)c1cc2[nH]ncc2cc1[N+](=O)[O-] ; CN(C)CCN(C)c1cc2[nH]ncc2cc1N]

685 mg (2.60 mmol) N,N,N′-trimethyl-N′-(5-nitro-1H-indazol-6-yl)ethane-1,2-diamine (prepared according to intermediate example 151b) were transformed in analogy to intermediate example 94b to give after working up and purification 525 mg (86%) of the title compound.

### Example 151b

### N,N,N′-trimethyl-N′-(5-nitro-1H-indazol-6-yl)ethane-1,2-diamine

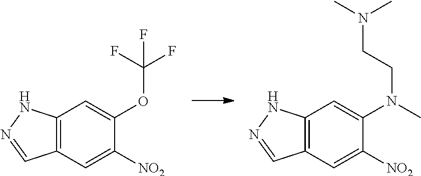 [SMILES: O=[N+]([O-])c1cc2cn[nH]c2cc1OC(F)(F)F ; CN(C)CCN(C)c1cc2[nH]ncc2cc1[N+](=O)[O-]]

1.00 g (4.05 mmol) 5-nitro-6-(trifluoromethoxy)-1H-indazole (prepared according to intermediate example 98b) were transformed in analogy to intermediate example 99b using N,N,N′-trimethylethane-1,2-diamine to give after working up and purification 730 mg (69%) of the title compound.

### Example 152

### (7S)-4-[(6-{[(2R)-1-Aminopropan-2-yl]oxy}-1H-indazol-5-yl)amino]-N,N-dimethyl-5,6,7,8-tetrahydro[1]benzothieno[2,3-d]pyrimidine-7-carboxamide

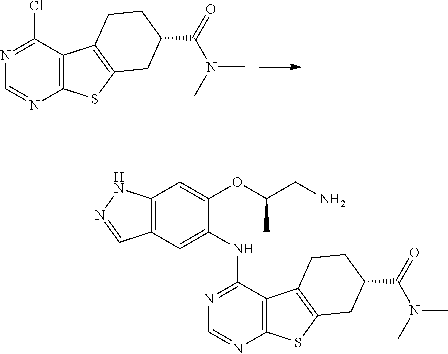 [SMILES: CN(C)C(=O)[C@H]1CCc2c(sc3ncnc(Cl)c23)C1 ; C[C@H](CN)Oc1cc2[nH]ncc2cc1Nc1ncnc2sc3c(c12)CC[C@H](C(=O)N(C)C)C3]

50 mg (169 μmol) (7S)-4-chloro-N,N-dimethyl-5,6,7,8-tetrahydro[1]benzothieno[2,3-d]pyrimidine-7-carboxamide (prepared according to intermediate example 94a) were transformed in analogy to intermediate example 130 using tert-butyl {(2R)-2-[(5-amino-1H-indazol-6-yl)oxy]propyl}carbamate (prepared according to intermediate example 140a) to give after working up and purification 37 mg (45%) of the title compound.

¹H-NMR (DMSO-d6): δ=1.37 (3H), 1.83 (1H), 2.17 (1H), 3.28 (1H), 2.88 (3H), 2.94 (2H), 3.06 (1H), 3.11 (3H), 3.18 (2H), 3.45 (1H), 5.02 (1H), 7.20 (1H), 8.02 (1H), 8.14 (2H), 8.28 (1H), 8.52 (1H), 9.03 (1H), 12.88 (1H) ppm.

### Example 153

### [(7S)-4-({6-[4-(Dimethylamino)piperidin-1-yl]-1H-indazol-5-yl}amino)-5,6,7,8-tetrahydro[1]benzothieno[2,3-d]pyrimidin-7-yl][(1S,4S)-2-oxa-5-azabicyclo[2.2.1]hept-5-yl]methanone

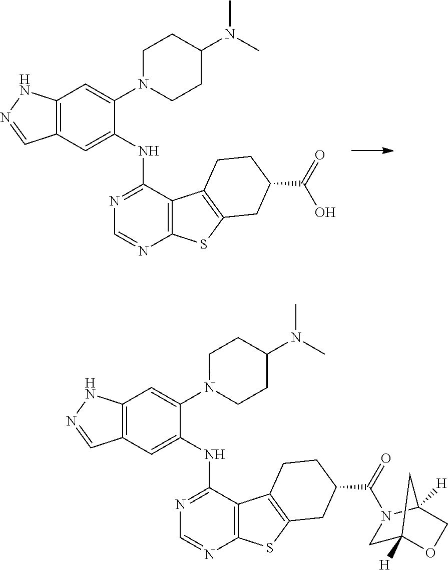 [SMILES: CN(C)C1CCN(c2cc3[nH]ncc3cc2Nc2ncnc3sc4c(c23)CC[C@H](C(=O)O)C4)CC1 ; CN(C)C1CCN(c2cc3[nH]ncc3cc2Nc2ncnc3sc4c(c23)CC[C@H](C(=O)N2C[C@@H]3C[C@H]2CO3)C4)CC1]

50 mg (102 μmol) (7S)-4-({6-[4-(dimethylamino)piperidin-1-yl]-1H-indazol-5-yl}amino)-5,6,7,8-tetrahydro[1]benzothieno[2,3-d]pyrimidine-7-carboxylic acid (prepared according to intermediate example 132a) were transformed in analogy to example 1 using (1S,4S)-2-oxa-5-azabicyclo[2.2.1]heptane to give after working up and purification 9.2 mg (15%) of the title compound.

¹H-NMR (DMSO-d6): δ=1.44-1.68 (2H), 1.74-1.99 (5H), 2.12 (1H), 2.22 (6H), 2.26 (1H), 2.70-3.79 (13H), 4.61+4.66 (1H), 4.77+4.85 (1H), 7.46 (1H), 8.03 (1H), 8.52 (1H), 9.01+9.02 (1H), 9.08 (1H), 12.89 (1H) ppm.

### Example 154

### [(7S)-4-({6-[4-(Dimethylamino)piperidin-1-yl]-1H-indazol-5-yl}amino)-5,6,7,8-tetrahydro[1]benzothieno[2,3-d]pyrimidin-7-yl][(1R,4R)-2-oxa-5-azabicyclo[2.2.1]hept-5-yl]methanone

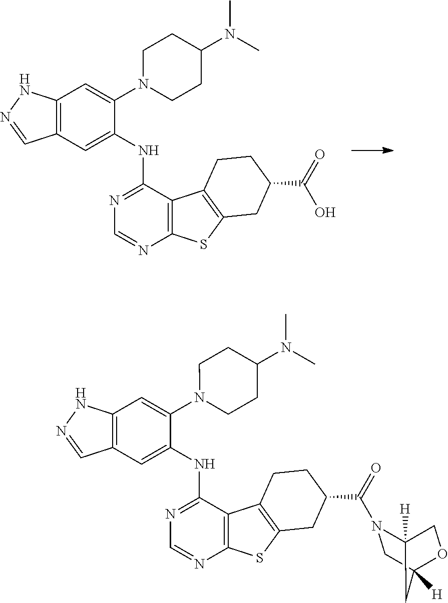 [SMILES: CN(C)C1CCN(c2cc3[nH]ncc3cc2Nc2ncnc3sc4c(c23)CC[C@H](C(=O)O)C4)CC1 ; CN(C)C1CCN(c2cc3[nH]ncc3cc2Nc2ncnc3sc4c(c23)CC[C@H](C(=O)N2C[C@H]3C[C@@H]2CO3)C4)CC1]

50 mg (102 μmol) (7S)-4-({6-[4-(dimethylamino)piperidin-1-yl]-1H-indazol-5-yl}amino)-5,6,7,8-tetrahydro[1]benzothieno[2,3-d]pyrimidine-7-carboxylic acid (prepared according to intermediate example 132a) were transformed in analogy to example 1 using (1R,4R)-2-oxa-5-azabicyclo[2.2.1]heptane to give after working up and purification 6.6 mg (11%) of the title compound.

¹H-NMR (DMSO-d6): δ=1.46-1.99 (6H), 2.07-2.37 (3H), 2.23 (6H), 2.68-3.79 (13H), 4.61+4.66 (1H), 4.77 (1H), 7.48 (1H), 8.03 (1H), 8.52 (1H), 9.04+9.07 (1H), 9.13 (1H), 12.90 (1H) ppm.

### Example 155

### (7S)-4-({6-[4-(Dimethylamino)piperidin-1-yl]-1H-indazol-5-yl}amino)-N-methyl-N-(propan-2-yl)-5,6,7,8-tetrahydro[1]benzothieno[2,3-d]pyrimidine-7-carboxamide

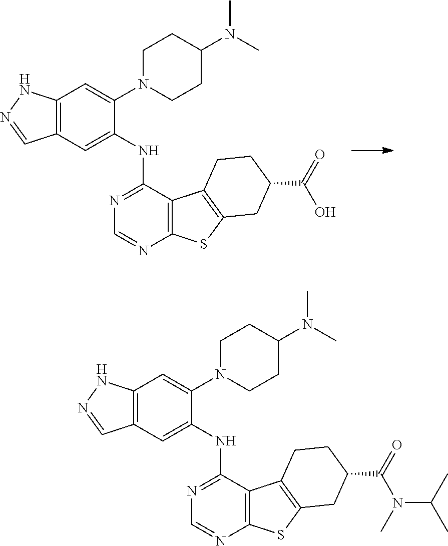 [SMILES: CN(C)C1CCN(c2cc3[nH]ncc3cc2Nc2ncnc3sc4c(c23)CC[C@H](C(=O)O)C4)CC1 ; CC(C)N(C)C(=O)[C@H]1CCc2c(sc3ncnc(Nc4cc5cn[nH]c5cc4N4CCC(N(C)C)CC4)c23)C1]

50 mg (102 μmol) (7S)-4-({6-[4-(dimethylamino)piperidin-1-yl]-1H-indazol-5-yl}amino)-5,6,7,8-tetrahydro[1]benzothieno[2,3-d]pyrimidine-7-carboxylic acid (prepared according to intermediate example 132a) were transformed in analogy to example 1 using N-methylpropan-2-amine to give after working up and purification 8.4 mg (14%) of the title compound.

¹H-NMR (DMSO-d6): δ=1.04 (3H), 1.17 (3H), 1.60 (2H), 1.78-1.97 (3H), 2.11 (1H), 2.22 (6H), 2.29 (1H), 2.71+2.88 (3H), 2.73-3.45 (11H), 4.25+4.72 (1H), 7.47 (1H), 8.03 (1H), 8.52 (1H), 9.02+9.04 (1H), 9.06+9.12 (1H), 12.90 (1H) ppm.

### Example 156

### (7S)-4-({6-[4-(Dimethylamino)piperidin-1-yl]-1H-indazol-5-yl}amino)-N-methyl-N-propyl-5,6,7,8-tetrahydro[1]benzothieno[2,3-d]pyrimidine-7-carboxamide

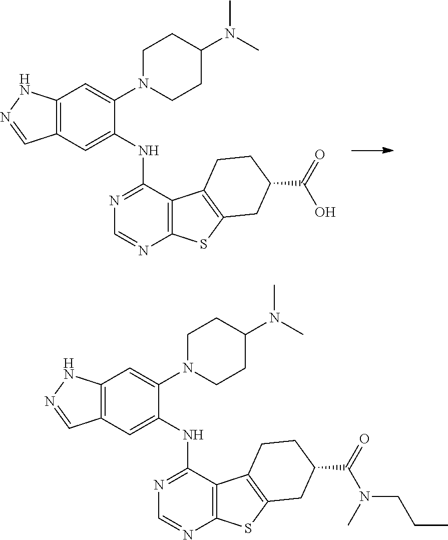 [SMILES: CN(C)C1CCN(c2cc3[nH]ncc3cc2Nc2ncnc3sc4c(c23)CC[C@H](C(=O)O)C4)CC1 ; CCCN(C)C(=O)[C@H]1CCc2c(sc3ncnc(Nc4cc5cn[nH]c5cc4N4CCC(N(C)C)CC4)c23)C1]

50 mg (102 μmol) (7S)-4-({6-[4-(dimethylamino)piperidin-1-yl]-1H-indazol-5-yl}amino)-5,6,7,8-tetrahydro[1]benzothieno[2,3-d]pyrimidine-7-carboxylic acid (prepared according to intermediate example 132a) were transformed in analogy to example 1 using N-methylpropan-1-amine to give after working up and purification 8.4 mg (14%) of the title compound.

¹H-NMR (DMSO-d6): δ=1.04 (3H), 1.17 (3H), 1.60 (2H), 1.77-1.97 (3H), 2.11 (1H), 2.22 (6H), 2.30 (1H), 2.71+2.88 (3H), 2.72-3.46 (9H), 4.25+4.72 (1H), 7.47 (1H), 8.03 (1H), 8.52 (1H), 9.02+9.04 (1H), 9.06+9.12 (1H), 12.90 (1H) ppm.

### Example 157

### (7S)-4-({6-[4-(Dimethylamino)piperidin-1-yl]-1H-indazol-5-yl}amino)-N-ethyl-N-(2-methoxyethyl)-5,6,7,8-tetrahydro[1]benzothieno[2,3-d]pyrimidine-7-carboxamide

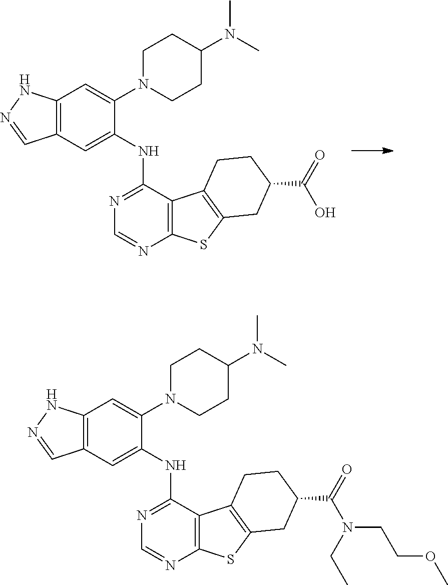 [SMILES: CN(C)C1CCN(c2cc3[nH]ncc3cc2Nc2ncnc3sc4c(c23)CC[C@H](C(=O)O)C4)CC1 ; CCN(CCOC)C(=O)[C@H]1CCc2c(sc3ncnc(Nc4cc5cn[nH]c5cc4N4CCC(N(C)C)CC4)c23)C1]

50 mg (102 μmol) (7S)-4-({6-[4-(dimethylamino)piperidin-1-yl]-1H-indazol-5-yl}amino)-5,6,7,8-tetrahydro[1]benzothieno[2,3-d]pyrimidine-7-carboxylic acid (prepared according to intermediate example 132a) were transformed in analogy to example 1 using N-ethyl-2-methoxyethanamine to give after working up and purification 7.4 mg (12%) of the title compound.

¹H-NMR (DMSO-d6): δ=1.02+1.13 (3H), 1.39-1.72 (3H), 1.78-1.99 (3H), 2.09 (1H), 2.23 (6H), 2.34 (1H), 2.68-3.63 (14H), 3.25+3.26 (3H), 7.47 (1H), 8.03 (1H), 8.52 (1H), 9.02+9.04 (1H), 9.07+9.10 (1H), 12.90 (1H) ppm.

### Example 158

### (7S)-4-({6-[4-(Dimethylamino)piperidin-1-yl]-1H-indazol-5-yl}amino)-N-ethyl-N-methyl-5,6,7,8-tetrahydro[1]benzothieno[2,3-d]pyrimidine-7-carboxamide

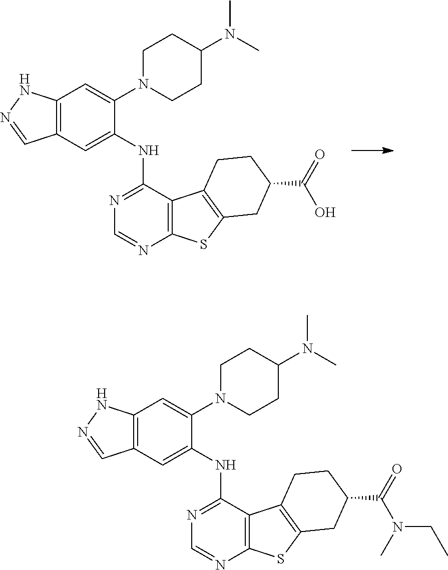 [SMILES: CN(C)C1CCN(c2cc3[nH]ncc3cc2Nc2ncnc3sc4c(c23)CC[C@H](C(=O)O)C4)CC1 ; CCN(C)C(=O)[C@H]1CCc2c(sc3ncnc(Nc4cc5cn[nH]c5cc4N4CCC(N(C)C)CC4)c23)C1]

50 mg (102 μmol) (7S)-4-({6-[4-(dimethylamino)piperidin-1-yl]-1H-indazol-5-yl}amino)-5,6,7,8-tetrahydro[1]benzothieno[2,3-d]pyrimidine-7-carboxylic acid (prepared according to intermediate example 132a) were transformed in analogy to example 1 using N-methylethanamine to give after working up and purification 17.8 mg (31%) of the title compound.

¹H-NMR (DMSO-d6): δ=1.02+1.13 (3H), 1.56-2.18 (6H), 2.46 (6H), 2.70-3.55 (12H), 2.84+3.05 (3H), 7.46 (1H), 8.03 (1H), 8.50 (1H), 8.92 (1H), 8.95 (1H), 12.92 (1H) ppm.

### Example 159

### (3-Hydroxyazetidin-1-yl){(7S)-4-[(6-propoxy-1H-indazol-5-yl)amino]-5,6,7,8-tetrahydro[1]benzothieno[2,3-d]pyrimidin-7-yl}methanone

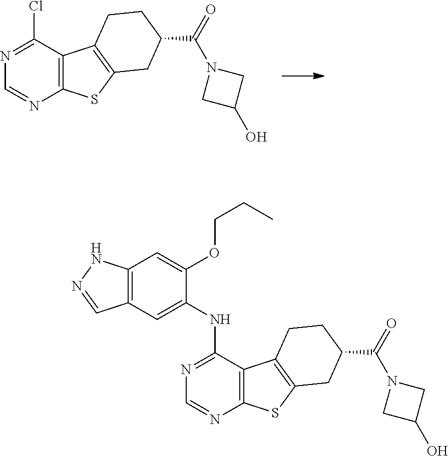 [SMILES: O=C([C@H]1CCc2c(sc3ncnc(Cl)c23)C1)N1CC(O)C1 ; CCCOc1cc2[nH]ncc2cc1Nc1ncnc2sc3c(c12)CC[C@H](C(=O)N1CC(O)C1)C3]

40 mg (124 μmol) [(7S)-4-chloro-5,6,7,8-tetrahydro[1]benzothieno[2,3-d]pyrimidin-7-yl](3-hydroxyazetidin-1-yl)methanone (prepared according to intermediate example 159a) were transformed in analogy to intermediate example 1b using 6-propoxy-1H-indazol-5-amine (prepared according to intermediate example 159b) to give after working up and purification 12.2 mg (22%) of the title compound.

¹H-NMR (DMSO-d6): δ=1.05 (3H), 1.77-1.95 (3H), 2.06 (1H), 2.79 (1H), 2.89 (2H), 3.19 (2H), 3.62 (1H), 3.89-4.18 (4H), 4.34-4.53 (2H), 5.72 (1H), 7.08 (1H), 7.99 (1H), 8.31 (1H), 8.51 (1H), 8.99 (1H), 12.81 (1H) ppm.

### Example 159a

### [(7S)-4-Chloro-5,6,7,8-tetrahydro[1]benzothieno[2,3-d]pyrimidin-7-yl](3-hydroxyazetidin-1-yl)methanone

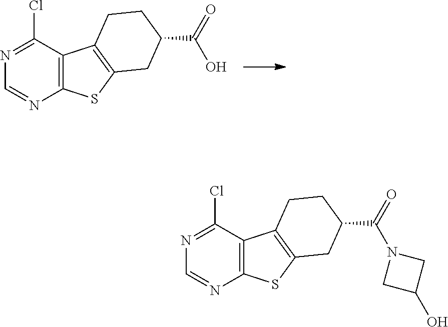 [SMILES: O=C(O)[C@H]1CCc2c(sc3ncnc(Cl)c23)C1 ; O=C([C@H]1CCc2c(sc3ncnc(Cl)c23)C1)N1CC(O)C1]

100 mg (372 μmol) (7S)-4-chloro-5,6,7,8-tetrahydro[1]benzothieno[2,3-d]pyrimidine-7-carboxylic acid (prepared according to intermediate example 66b) were transformed in analogy to example 1 using azetidin-3-ol to give after working up and purification 37 mg (31%) of the title compound.

### Example 159b

### 6-Propoxy-1H-indazol-5-amine

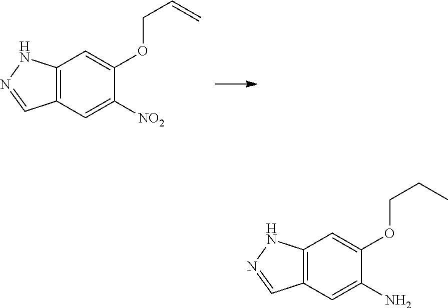 [SMILES: C=CCOc1cc2[nH]ncc2cc1[N+](=O)[O-] ; CCCOc1cc2[nH]ncc2cc1N]

500 mg (2.28 mmol) 6-(allyloxy)-5-nitro-1H-indazole (prepared according to intermediate example 94c) were transformed in analogy to intermediate example 94b to give after working up and purification 430 mg (99%) of the title compound.

### Example 160

### [(2R,6S)-2,6-dimethylmorpholin-4-yl][(7S)-4-{[6-(propan-2-yloxy)-1H-indazol-5-yl]amino}-5,6,7,8-tetrahydro[1]benzothieno[2,3-d]pyrimidin-7-yl]methanone

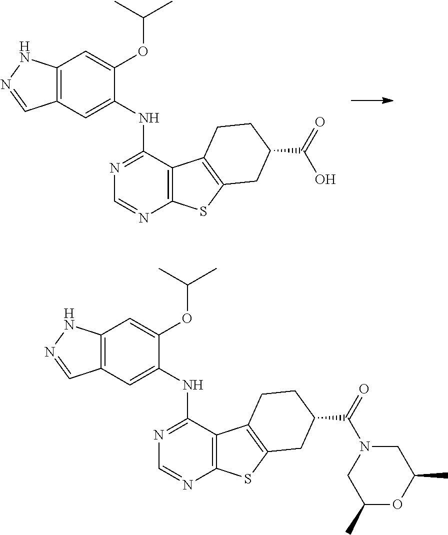 [SMILES: CC(C)Oc1cc2[nH]ncc2cc1Nc1ncnc2sc3c(c12)CC[C@H](C(=O)O)C3 ; CC(C)Oc1cc2[nH]ncc2cc1Nc1ncnc2sc3c(c12)CC[C@H](C(=O)N1C[C@@H](C)O[C@@H](C)C1)C3]

100 mg (236 μmol) (7S)-4-[(6-isopropoxy-1H-indazol-5-yl)amino]-5,6,7,8-tetrahydro[1]benzothieno[2,3-d]pyrimidine-7-carboxylic acid (prepared according to intermediate example 78a) were transformed in analogy to example 1 using (2R,6S)-2,6-dimethylmorpholine to give after working up and purification 65.1 mg (50%) of the title compound.

¹H-NMR (DMSO-d6): δ=1.10 (6H), 1.41 (6H), 1.89 (1H), 2.06 (1H), 2.27 (1H), 2.76 (1H), 2.93 (2H), 3.18-3.33 (3H), 3.44 (1H), 3.52 (1H), 3.98 (1H), 4.31 (1H), 4.88 (1H), 7.11 (1H), 7.99 (1H), 8.37 (1H), 8.52 (1H), 9.05+9.07 (1H), 12.76 (1H) ppm.

### Example 161

### [(2R,6S)-2, 6-dimethylmorpholin-4-yl]{(7S)-4-[(6-methoxy-1H-indazol-5-yl)amino]-5,6,7,8-tetrahydro[1]benzothieno[2,3-d]pyrimidin-7-yl}methanone

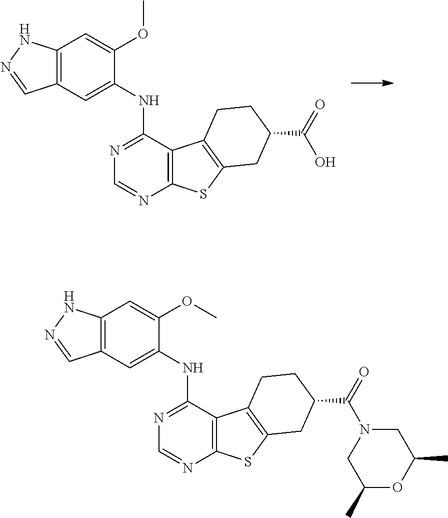 [SMILES: COc1cc2[nH]ncc2cc1Nc1ncnc2sc3c(c12)CC[C@H](C(=O)O)C3 ; COc1cc2[nH]ncc2cc1Nc1ncnc2sc3c(c12)CC[C@H](C(=O)N1C[C@@H](C)O[C@@H](C)C1)C3]

100 mg (253 μmol) (7S)-4-[(6-methoxy-1H-indazol-5-yl)amino]-5,6,7,8-tetrahydro[1]benzothieno[2,3-d]pyrimidine-7-carboxylic acid (prepared according to intermediate example 20a) were transformed in analogy to example 1 using (2R,6S)-2,6-dimethylmorpholine to give after working up and purification 76.8 mg (62%) of the title compound.

¹H-NMR (DMSO-d6): δ=1.11 (6H), 1.87 (1H), 2.11 (1H), 2.27 (1H), 2.76 (1H), 2.85-3.26 (5H), 3.43 (1H), 3.53 (1H), 3.97 (3H), 4.01 (1H), 4.31 (1H), 7.08 (1H), 7.99 (1H), 8.20 (1H), 8.45 (1H), 8.75 (1H), 12.83 (1H) ppm.

### Example 162

### [(2R,6S)-2, 6-dimethylmorpholin-4-yl][(7S)-4-(1H-indazol-5-ylamino)-5,6,7,8-tetrahydro[1]benzothieno[2,3-d]pyrimidin-7-yl]methanone

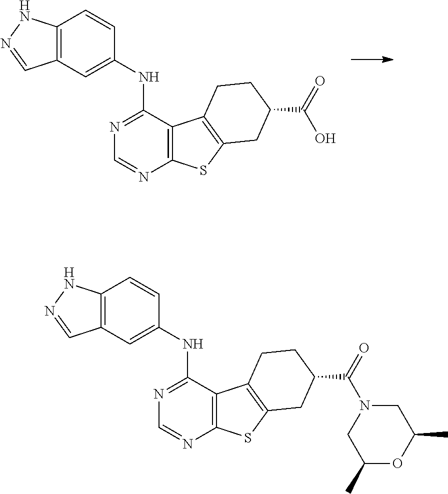 [SMILES: O=C(O)[C@H]1CCc2c(sc3ncnc(Nc4ccc5[nH]ncc5c4)c23)C1 ; C[C@@H]1CN(C(=O)[C@H]2CCc3c(sc4ncnc(Nc5ccc6[nH]ncc6c5)c34)C2)C[C@H](C)O1]

91 mg (249 μmol) (7S)-4-(1H-indazol-5-ylamino)-5,6,7,8-tetrahydro[1]benzothieno[2,3-d]pyrimidine-7-carboxylic acid (prepared according to intermediate example 1a) were transformed in analogy to example 1 using (2R,6S)-2,6-dimethylmorpholine to give after working up and purification 19.1 mg (16%) of the title compound.

¹H-NMR (DMSO-d6): δ=1.10 (6H), 1.79 (1H), 2.04 (1H), 2.26 (1H), 2.77 (1H), 2.85-3.04 (2H), 3.14-3.27 (3H), 3.44 (1H), 3.52 (1H), 3.94 (1H), 4.31 (1H), 7.46-7.53 (2H), 7.98 (1H), 8.04 (1H), 8.18 (1H), 8.30 (1H), 13.01 (1H) ppm.

### Example 163

### (3-fluoroazetidin-1-yl){(7S)-4-[(6-methoxy-1H-indazol-5-yl)amino]-5,6,7,8-tetrahydro[1]benzothieno[2,3-d]pyrimidin-7-yl}methanone

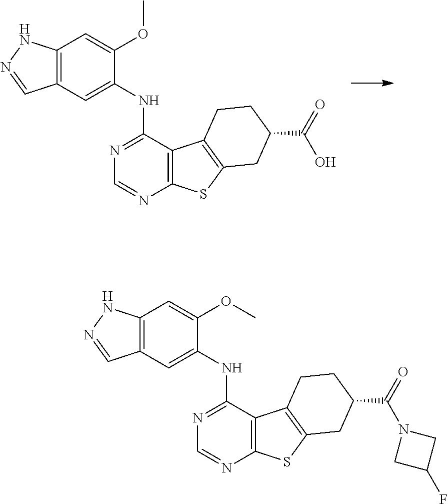 [SMILES: COc1cc2[nH]ncc2cc1Nc1ncnc2sc3c(c12)CC[C@H](C(=O)O)C3 ; COc1cc2[nH]ncc2cc1Nc1ncnc2sc3c(c12)CC[C@H](C(=O)N1CC(F)C1)C3]

70 mg (177 μmol) (7S)-4-[(6-methoxy-1H-indazol-5-yl)amino]-5,6,7,8-tetrahydro[1]benzothieno[2,3-d]pyrimidine-7-carboxylic acid (prepared according to intermediate example 20a) were transformed in analogy to example 1 using 3-fluoroazetidine to give after working up and purification 43.8 mg (52%) of the title compound.

¹H-NMR (DMSO-d6): δ=1.82 (1H), 2.16 (1H), 2.75-3.00 (3H), 3.07-3.41 (2H), 3.93 (1H), 3.98 (3H), 4.14-4.44 (2H), 4.58 (1H), 5.34+5.52 (1H), 7.09 (1H), 7.99 (1H), 8.21 (1H), 8.45 (1H), 8.77 (1H), 12.84 (1H) ppm.

### Example 164

### (3,3-difluoroazetidin-1-yl){(7S)-4-[(6-methoxy-1H-indazol-5-yl)amino]-5,6,7,8-tetrahydro[1]benzothieno[2,3-d]pyrimidin-7-yl}methanone

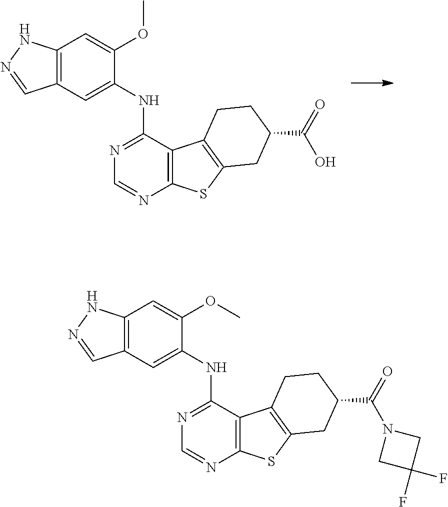 [SMILES: COc1cc2[nH]ncc2cc1Nc1ncnc2sc3c(c12)CC[C@H](C(=O)O)C3 ; COc1cc2[nH]ncc2cc1Nc1ncnc2sc3c(c12)CC[C@H](C(=O)N1CC(F)(F)C1)C3]

70 mg (177 μmol) (7S)-4-[(6-methoxy-1H-indazol-5-yl)amino]-5,6,7,8-tetrahydro[1]benzothieno[2,3-d]pyrimidine-7-carboxylic acid (prepared according to intermediate example 20a) were transformed in analogy to example 1 using 3,3-difluoroazetidine to give after working up and purification 44.1 mg (50%) of the title compound.

¹H-NMR (DMSO-d6): δ=1.85 (1H), 2.21 (1H), 2.81-3.04 (3H), 3.15 (1H), 3.25 (1H), 3.98 (3H), 4.33 (2H), 4.76 (2H), 7.09 (1H), 7.99 (1H), 8.22 (1H), 8.46 (1H), 8.77 (1H), 12.84 (1H) ppm.

### Example 165

### tert-butyl {2-[(5-{[(7S)-7-(dimethylcarbamoyl)-5,6,7,8-tetrahydro[1]benzothieno[2,3-d]pyrimidin-4-yl]amino}-1H-indazol-6-yl)oxy]ethyl}carbamate

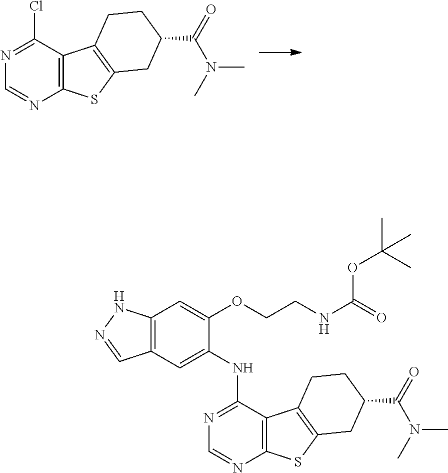 [SMILES: CN(C)C(=O)[C@H]1CCc2c(sc3ncnc(Cl)c23)C1 ; CN(C)C(=O)[C@H]1CCc2c(sc3ncnc(Nc4cc5cn[nH]c5cc4OCCNC(=O)OC(C)(C)C)c23)C1]

100 mg (338 μmol) (7S)-4-chloro-N,N-dimethyl-5,6,7,8-tetrahydro[1]benzothieno[2,3-d]pyrimidine-7-carboxamide (prepared according to intermediate example 94a) were transformed in analogy to intermediate example 1b using tert-butyl {2-[(5-amino-1H-indazol-6-yl)oxy]ethyl}carbamate (prepared according to intermediate example 130a) to give after working up and purification 80.1 mg (43%) of the title compound.

¹H-NMR (DMSO-d6): δ=1.31 (9H), 1.81 (1H), 2.12 (1H), 2.86 (3H), 2.92 (2H), 3.10 (3H), 3.15 (1H), 3.22-3.37 (2H), 3.45 (2H), 4.20 (2H), 7.07 (1H), 7.15 (1H), 7.99 (1H), 8.28 (1H), 8.51 (1H), 8.96 (1H), 12.81 (1H) ppm.

### Example 166

### [(7S)-4-({6-[4-(dimethylamino)piperidin-1-yl]-1H-indazol-5-yl}amino)-5,6,7,8-tetrahydro[1]benzothieno[2,3-d]pyrimidin-7-yl](2-oxa-6-azaspiro[3.3]hept-6-yl)methanone

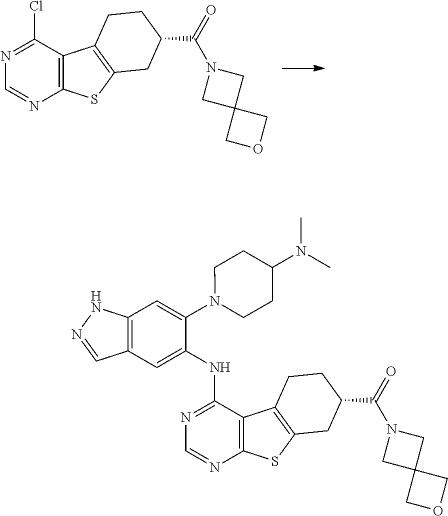 [SMILES: O=C([C@H]1CCc2c(sc3ncnc(Cl)c23)C1)N1CC2(COC2)C1 ; CN(C)C1CCN(c2cc3[nH]ncc3cc2Nc2ncnc3sc4c(c23)CC[C@H](C(=O)N2CC3(COC3)C2)C4)CC1]

100 mg (286 μmol) [(7S)-4-chloro-5,6,7,8-tetrahydro[1]benzothieno[2,3-d]pyrimidin-7-yl](2-oxa-6-azaspiro[3.3]hept-6-yl)methanone (prepared according to intermediate example 166a) were transformed in analogy to intermediate example 1b using 6-[4-(dimethylamino)piperidin-1-yl]-1H-indazol-5-amine (prepared according to intermediate example 104a) to give after working up and purification 17.1 mg (9%) of the title compound.

¹H-NMR (DMSO-d6): δ=1.58 (2H), 1.81 (1H), 1.91 (2H), 2.10 (1H), 2.24 (6H), 2.28 (1H), 2.71-2.86 (3H), 2.90 (2H), 3.05 (2H), 3.36 (2H), 4.05 (2H), 4.31 (1H), 4.43 (1H), 4.63-4.72 (4H), 7.46 (1H), 8.03 (1H), 8.51 (1H), 8.99 (1H), 9.04 (1H), 12.91 (1H) ppm.

### Example 166a

### [(7S)-4-Chloro-5,6,7,8-tetrahydro[1]benzothieno[2,3-d]pyrimidin-7-yl](2-oxa-6-azaspiro[3.3]hept-6-yl)methanone

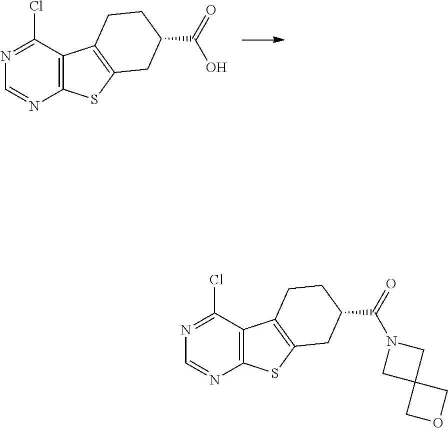 [SMILES: O=C(O)[C@H]1CCc2c(sc3ncnc(Cl)c23)C1 ; O=C([C@H]1CCc2c(sc3ncnc(Cl)c23)C1)N1CC2(COC2)C1]

500 mg (1.86 mmol) (7S)-4-chloro-5,6,7,8-tetrahydro[1]benzothieno[2,3-d]pyrimidine-7-carboxylic acid (prepared according to intermediate example 66b) were transformed in analogy to example 1 using 2-oxa-6-azaspiro[3.3]heptane to give after working up and purification 362 mg (56%) of the title compound.

### Example 167

### [(2R,6S)-2, 6-dimethylmorpholin-4-yl]{(7S)-4-[(6-ethoxy-1H-indazol-5-yl)amino]-5,6,7,8-tetrahydro[1]benzothieno[2,3-d]pyrimidin-7-yl}methanone

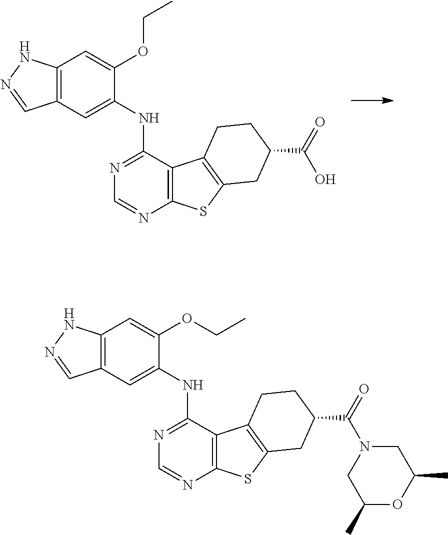 [SMILES: CCOc1cc2[nH]ncc2cc1Nc1ncnc2sc3c(c12)CC[C@H](C(=O)O)C3 ; CCOc1cc2[nH]ncc2cc1Nc1ncnc2sc3c(c12)CC[C@H](C(=O)N1C[C@@H](C)O[C@@H](C)C1)C3]

50 mg (122 μmol) (7S)-4-[(6-ethoxy-1H-indazol-5-yl)amino]-5,6,7,8-tetrahydro[1]benzothieno[2,3-d]pyrimidine-7-carboxylic acid (prepared according to intermediate example 65a) were transformed in analogy to example 1 using (2R,6S)-2,6-dimethylmorpholine to give after working up and purification 17.8 mg (26%) of the title compound.

¹H-NMR (DMSO-d6): δ=1.12 (6H), 1.48 (3H), 1.88 (1H), 2.07 (1H), 2.27 (1H), 2.76 (1H), 2.94 (2H), 3.15-3.59 (5H), 3.98 (1H), 4.23 (2H), 4.30 (1H), 7.07 (1H), 7.99 (1H), 8.37 (1H), 8.53 (1H), 9.00+9.02 (1H), 12.81 (1H) ppm.

### Example 168

### [(3S)-3-(dimethylamino)pyrrolidin-1-yl][(7S)-4-{[6-(propan-2-yloxy)-1H-indazol-5-yl]amino}-5,6,7,8-tetrahydro[1]benzothieno[2,3-d]pyrimidin-7-yl]methanone

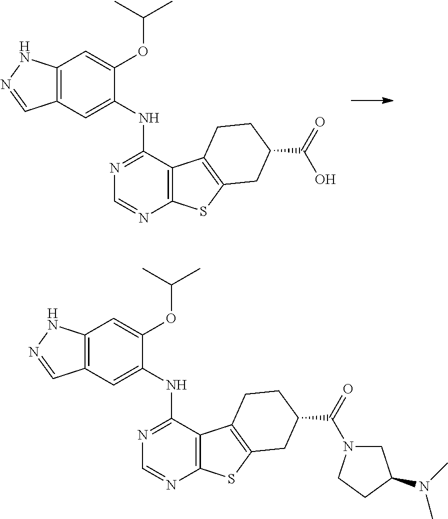 [SMILES: CC(C)Oc1cc2[nH]ncc2cc1Nc1ncnc2sc3c(c12)CC[C@H](C(=O)O)C3 ; CC(C)Oc1cc2[nH]ncc2cc1Nc1ncnc2sc3c(c12)CC[C@H](C(=O)N1CC[C@H](N(C)C)C1)C3]

100 mg (236 μmol) (7S)-4-[(6-isopropoxy-1H-indazol-5-yl)amino]-5,6,7,8-tetrahydro[1]benzothieno[2,3-d]pyrimidine-7-carboxylic acid (prepared according to intermediate example 78a) were transformed in analogy to example 1 using (3S)—N,N-dimethylpyrrolidin-3-amine to give after working up and purification 85.5 mg (66%) of the title compound.

¹H-NMR (DMSO-d6): δ=1.41 (6H), 1.57-1.94 (2H), 1.74-1.93 (2H), 1.98-2.14 (2H), 2.17 (6H), 2.61+2.72 (1H), 2.85-3.10 (3H), 3.14-3.27 (2H), 3.51-3.84 (2H), 4.88 (1H), 7.11 (1H), 7.99 (1H), 8.37 (1H), 8.53 (1H), 9.06 (1H), 12.76 (1H) ppm.

### Example 169

### [(3R)-3-(Dimethylamino)pyrrolidin-1-yl][(7S)-4-{[6-(propan-2-yloxy)-1H-indazol-5-yl]amino}-5,6,7,8-tetrahydro[1]benzothieno[2,3-d]pyrimidin-7-yl]methanone

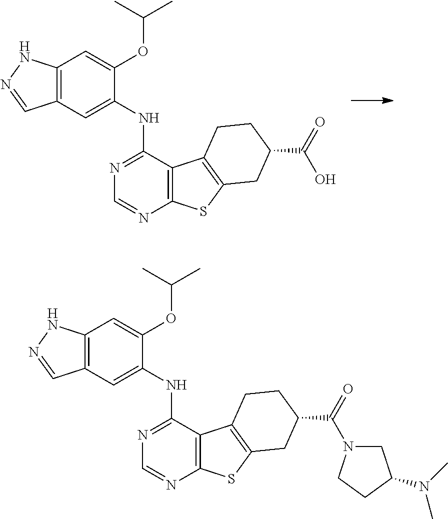 [SMILES: CC(C)Oc1cc2[nH]ncc2cc1Nc1ncnc2sc3c(c12)CC[C@H](C(=O)O)C3 ; CC(C)Oc1cc2[nH]ncc2cc1Nc1ncnc2sc3c(c12)CC[C@H](C(=O)N1CC[C@@H](N(C)C)C1)C3]

75 mg (177 μmol) (7S)-4-[(6-isopropoxy-1H-indazol-5-yl)amino]-5,6,7,8-tetrahydro[1]benzothieno[2,3-d]pyrimidine-7-carboxylic acid (prepared according to intermediate example 78a) were transformed in analogy to example 1 using (3R)—N,N-dimethylpyrrolidin-3-amine to give after working up and purification 24.8 mg (26%) of the title compound.

¹H-NMR (DMSO-d6): δ=1.41 (6H), 1.65+1.76 (1H), 1.87 (1H), 1.97-2.14 (2H), 2.16 (3H), 2.17 (3H), 2.66 (1H), 2.88-3.08 (4H), 3.16-3.66 (4H), 3.78+3.86 (1H), 4.89 (1H), 7.12 (1H), 7.99 (1H), 8.37+8.38 (1H), 8.53 (1H), 9.07 (1H), 12.76 (1H) ppm.

### Example 170

### (7S)—N-[3-(1H-Imidazol-1-yl)propyl]-4-[(6-methoxy-1H-indazol-5-yl)amino]-N-methyl-5,6,7,8-tetrahydro[1]benzothieno[2,3-d]pyrimidine-7-carboxamide

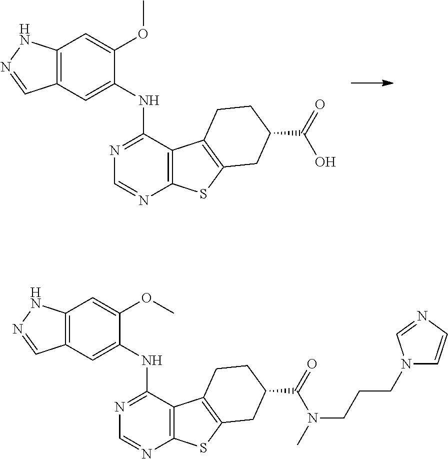 [SMILES: COc1cc2[nH]ncc2cc1Nc1ncnc2sc3c(c12)CC[C@H](C(=O)O)C3 ; COc1cc2[nH]ncc2cc1Nc1ncnc2sc3c(c12)CC[C@H](C(=O)N(C)CCCn1ccnc1)C3]

70 mg (177 μmol) (7S)-4-[(6-methoxy-1H-indazol-5-yl)amino]-5,6,7,8-tetrahydro[1]benzothieno[2,3-d]pyrimidine-7-carboxylic acid (prepared according to intermediate example 20a) were transformed in analogy to example 1 using 3-(1H-imidazol-1-yl)-N-methylpropan-1-amine to give after working up and purification 44.0 mg (46%) of the title compound.

¹H-NMR (DMSO-d6): δ=1.73-2.22 (4H), 2.84+3.08 (3H), 2.94 (2H), 3.09-3.42 (4H), 3.88-4.09 (6H), 6.82+6.88 (1H), 7.08+7.09 (1H), 7.21 (1H), 7.65 (1H), 7.99 (1H), 8.22 (1H), 8.46 (1H), 8.78 (1H), 12.85 (1H) ppm.

### Example 171

### (7S)—N-[2-(1H-Imidazol-1-yl)ethyl]-4-[(6-methoxy-1H-indazol-5-yl)amino]-N-methyl-5,6,7,8-tetrahydro[1]benzothieno[2,3-d]pyrimidine-7-carboxamide

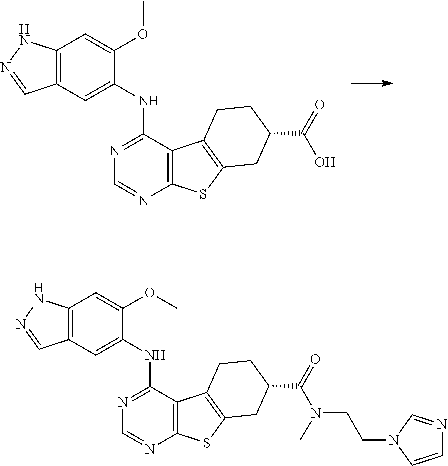 [SMILES: COc1cc2[nH]ncc2cc1Nc1ncnc2sc3c(c12)CC[C@H](C(=O)O)C3 ; COc1cc2[nH]ncc2cc1Nc1ncnc2sc3c(c12)CC[C@H](C(=O)N(C)CCn1ccnc1)C3]

70 mg (177 μmol) (7S)-4-[(6-methoxy-1H-indazol-5-yl)amino]-5,6,7,8-tetrahydro[1]benzothieno[2,3-d]pyrimidine-7-carboxylic acid (prepared according to intermediate example 20a) were transformed in analogy to example 1 using 2-(1H-imidazol-1-yl)-N-methylethanamine to give after working up and purification 46.6 mg (50%) of the title compound.

¹H-NMR (DMSO-d6): δ=1.63 (1H), 1.80+2.08 (1H), 2.53-3.23 (5H), 2.88+2.90 (3H), 3.53-3.88 (2H), 3.98+4.04 (3H), 4.12+4.19 (2H), 6.90 (1H), 7.09 (1H), 7.19+7.21 (1H), 7.61+7.63 (1H), 7.99 (1H), 8.20 (1H), 8.46 (1H), 8.76+8.80 (1H), 12.86 (1H) ppm.

### Example 172

### [(3S)-3-(Dimethylamino)pyrrolidin-1-yl]{(7S)-4-[(6-ethoxy-1H-indazol-5-yl)amino]-5,6,7,8-tetrahydro[1]benzothieno[2,3-d]pyrimidin-7-yl}methanone

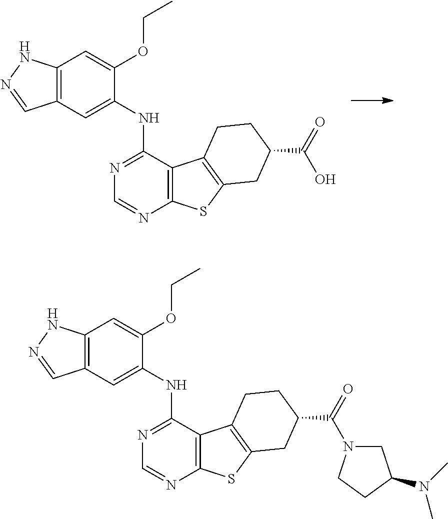 [SMILES: CCOc1cc2[nH]ncc2cc1Nc1ncnc2sc3c(c12)CC[C@H](C(=O)O)C3 ; CCOc1cc2[nH]ncc2cc1Nc1ncnc2sc3c(c12)CC[C@H](C(=O)N1CC[C@H](N(C)C)C1)C3]

70 mg (171 μmol) (7S)-4-[(6-ethoxy-1H-indazol-5-yl)amino]-5,6,7,8-tetrahydro[1]benzothieno[2,3-d]pyrimidine-7-carboxylic acid (prepared according to intermediate example 65a) were transformed in analogy to example 1 using (3S)—N,N-dimethylpyrrolidin-3-amine to give after working up and purification 49.1 mg (54%) of the title compound.

¹H-NMR (DMSO-d6): δ=1.47 (3H), 1.54-1.92 (2H), 1.96-2.23 (2H), 2.16 (6H), 2.40-3.86 (10H), 4.20 (2H), 7.05 (1H), 7.99 (1H), 8.35 (1H), 8.51 (1H), 9.01 (1H), 12.84 (1H) ppm.

### Example 173

### (7S)—N-(2, 2-Dimethylpropyl)-4-[(6-ethoxy-1H-indazol-5-yl)amino]-N-methyl-5,6,7,8-tetrahydro[1]benzothieno[2,3-d]pyrimidine-7-carboxamide

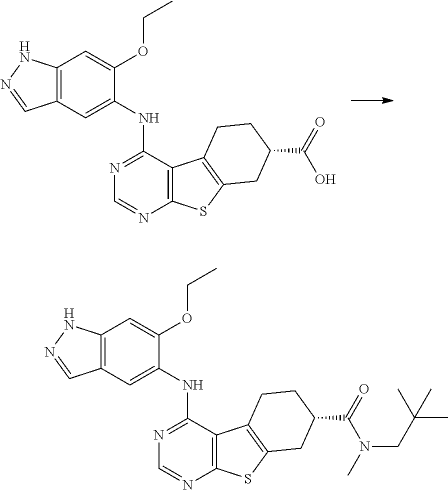 [SMILES: CCOc1cc2[nH]ncc2cc1Nc1ncnc2sc3c(c12)CC[C@H](C(=O)O)C3 ; CCOc1cc2[nH]ncc2cc1Nc1ncnc2sc3c(c12)CC[C@H](C(=O)N(C)CC(C)(C)C)C3]

70 mg (171 μmol) (7S)-4-[(6-ethoxy-1H-indazol-5-yl)amino]-5,6,7,8-tetrahydro[1]benzothieno[2,3-d]pyrimidine-7-carboxylic acid (prepared according to intermediate example 65a) were transformed in analogy to example 1 using N,2,2-trimethylpropan-1-amine to give after working up and purification 55.8 mg (63%) of the title compound.

¹H-NMR (DMSO-d6): δ=0.89+0.93 (9H), 1.46 (3H), 1.87 (1H), 2.08 (1H), 2.80-3.03 (3H), 3.16 (3H), 3.18-3.45 (4H), 4.20 (2H), 7.04 (1H), 7.99 (1H), 8.33 (1H), 8.51 (1H), 9.02 (1H), 12.82 (1H) ppm.

### Example 174

### (7S)-4-[(6-Ethoxy-1H-indazol-5-yl)amino]-N-methyl-N-propyl-5,6,7,8-tetrahydro[1]benzothieno[2,3-d]pyrimidine-7-carboxamide

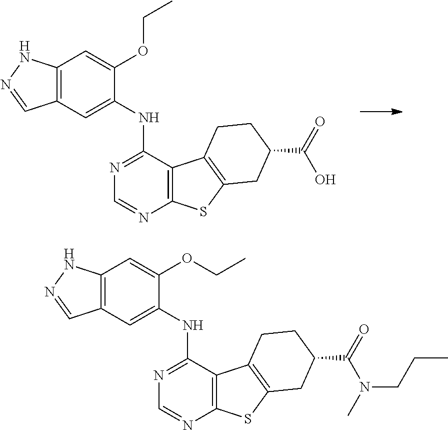

200 mg (488 μmol) (7S)-4-[(6-ethoxy-1H-indazol-5-yl)amino]-5,6,7,8-tetrahydro[1]benzothieno[2,3-d]pyrimidine-7-carboxylic acid (prepared according to intermediate example 65a) were transformed in analogy to example 1 using N-methylpropan-1-amine to give after working up and purification 76.2 mg (32%) of the title compound.

¹H-NMR (DMSO-d6): δ=0.83+0.87 (3H), 1.45 (3H), 1.46-1.63 (2H), 1.86 (1H), 2.03 (1H), 2.85+3.07 (3H), 2.91 (2H), 3.09-3.42 (5H), 4.18 (2H), 7.03 (1H), 7.98 (1H), 8.32 (1H), 8.51 (1H), 9.02 (1H), 12.82 (1H) ppm.

### Example 175

### {(7S)-4-[(6-Ethoxy-1H-indazol-5-yl)amino]-5,6,7,8-tetrahydro[1]benzothieno[2,3-d]pyrimidin-7-yl}[3-(piperidin-1-yl)azetidin-1-yl]methanone

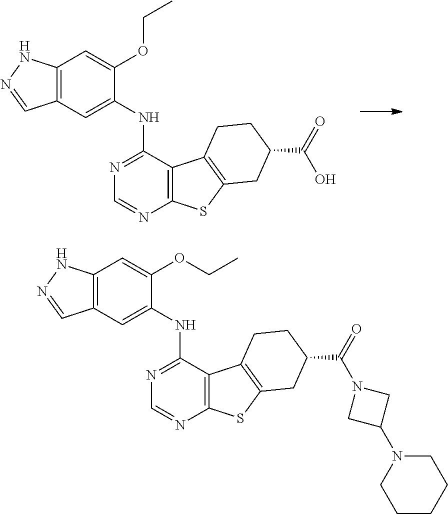

70 mg (171 μmol) (7S)-4-[(6-ethoxy-1H-indazol-5-yl)amino]-5,6,7,8-tetrahydro[1]benzothieno[2,3-d]pyrimidine-7-carboxylic acid (prepared according to intermediate example 65a) were transformed in analogy to example 1 using 1-(azetidin-3-yl)piperidine to give after working up and purification 37.4 mg (39%) of the title compound.

¹H-NMR (DMSO-d6): δ=1.34 (1H), 1.35-1.55 (9H), 1.83 (1H), 2.06 (1H), 2.23 (3H), 2.78 (1H), 2.84-2.99 (2H), 3.00-3.28 (3H), 3.63-4.28 (6H), 7.05 (1H), 7.99 (1H), 8.33 (1H), 8.51 (1H), 9.01 (1H), 12.80 (1H) ppm.

### Example 176

### (7S)-4-{[6-(2,2-Dimethylpropoxy)-1H-indazol-5-yl]amino}-N,N-dimethyl-5,6,7,8-tetrahydro[1]benzothieno[2,3-d]pyrimidine-7-carboxamide

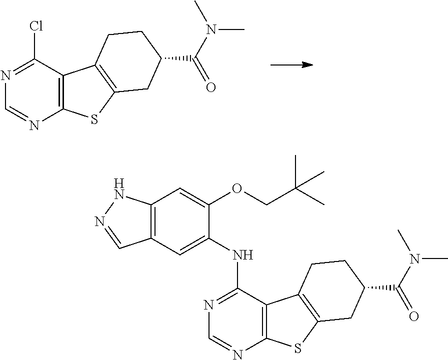 [SMILES: CN(C)C(=O)[C@H]1CCc2c(sc3ncnc(Cl)c23)C1 ; CN(C)C(=O)[C@H]1CCc2c(sc3ncnc(Nc4cc5cn[nH]c5cc4OCC(C)(C)C)c23)C1]

84.3 mg (285 μmol) (7S)-4-chloro-N,N-dimethyl-5,6,7,8-tetrahydro[1]benzothieno[2,3-d]pyrimidine-7-carboxamide (prepared according to intermediate example 94a) were transformed in analogy to intermediate example 1b using 6-(2,2-dimethylpropoxy)-1H-indazol-5-amine (prepared according to intermediate example 176a) to give after working up and purification 40.7 mg (27%) of the title compound.

¹H-NMR (DMSO-d6): δ=1.00 (9H), 1.82 (1H), 2.05 (1H), 2.88 (3H), 2.93 (2H), 3.09 (3H), 3.17-3.27 (3H), 3.81 (2H), 7.07 (1H), 7.98 (1H), 8.08 (1H), 8.43 (1H), 8.69 (1H), 12.80 (1H) ppm.

### Example 176a

### 6-(2,2-Dimethylpropoxy)-1H-indazol-5-amine

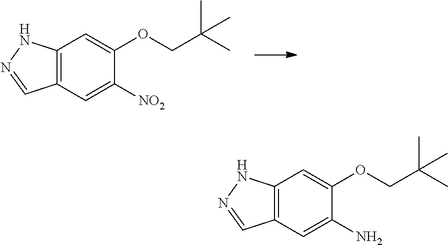 [SMILES: CC(C)(C)COc1cc2[nH]ncc2cc1[N+](=O)[O-] ; CC(C)(C)COc1cc2[nH]ncc2cc1N]

3.67 g (14.7 mmol) 6-(2,2-dimethylpropoxy)-5-nitro-1H-indazole (prepared according to intermediate example 176b) were transformed in analogy to intermediate example 94b to give after working up and purification 3.07 g (93%) of the title compound.

### Example 176b

### 6-(2,2-Dimethylpropoxy)-5-nitro-1H-indazole

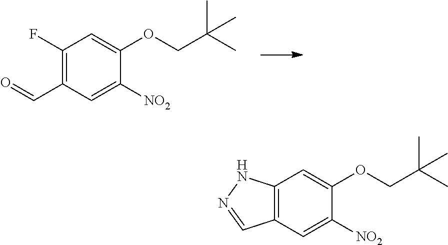 [SMILES: CC(C)(C)COc1cc(F)c(C=O)cc1[N+](=O)[O-] ; CC(C)(C)COc1cc2[nH]ncc2cc1[N+](=O)[O-]]

5.64 g (22.1 mmol) 4-(2,2-dimethylpropoxy)-2-fluoro-5-nitrobenzaldehyde (prepared according to intermediate example 176c) were transformed in analogy to intermediate example 98b to give after working up and purification 3.67 g (67%) of the title compound.

### Example 176c

### 4-(2,2-Dimethylpropoxy)-2-fluoro-5-nitrobenzaldehyde

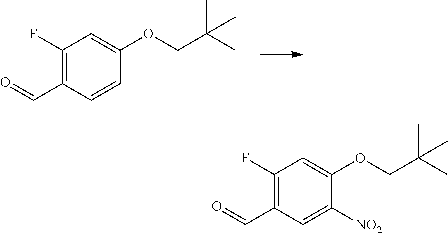 [SMILES: CC(C)(C)COc1ccc(C=O)c(F)c1 ; CC(C)(C)COc1cc(F)c(C=O)cc1[N+](=O)[O-]]

3.49 g (16.6 mol) 4-(2,2-dimethylpropoxy)-2-fluorobenzaldehyde (prepared according to intermediate example 176d) were transformed in analogy to intermediate example 94e to give after working up and purification 4.48 g (61%) of the title compound.

### Example 176d

### 4-(2,2-dimethylpropoxy)-2-fluorobenzaldehyde

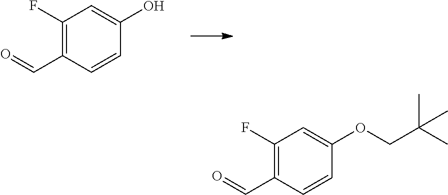

A mixture comprising 5.00 g (35.7 mmol) 2-fluoro-4-hydroxybenzaldehyde (CAS-No: 348-27-6), 5.04 mL 1-bromo-2,2-dimethylpropane, 11.63 g cesium carbonate and 35 mL N,N-dimethylformamide was heated at 150° C. for 90 minutes under microwave irradiation. The mixture was poured into water and extracted with ethyl acetate. The organic layer was washed with brine and dried over sodium sulfate. After filtration and removal of the solvents the residue was purified by chromatography to give 3.49 g (46%) of the title compound.

### Example 177

### (7S)—N-(2, 2-Dimethylpropyl)-4-[(6-methoxy-1H-indazol-5-yl)amino]-N-methyl-5,6,7,8-tetrahydro[1]benzothieno[2,3-d]pyrimidine-7-carboxamide

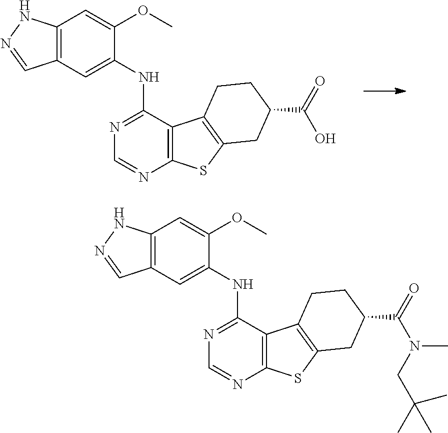 [SMILES: COc1cc2[nH]ncc2cc1Nc1ncnc2sc3c(c12)CC[C@H](C(=O)O)C3 ; COc1cc2[nH]ncc2cc1Nc1ncnc2sc3c(c12)CC[C@H](C(=O)N(C)CC(C)(C)C)C3]

70 mg (177 μmol) (7S)-4-[(6-methoxy-1H-indazol-5-yl)amino]-5,6,7,8-tetrahydro[1]benzothieno[2,3-d]pyrimidine-7-carboxylic acid (prepared according to intermediate example 20a) were transformed in analogy to example 1 using N,2,2-trimethylpropan-1-amine to give after working up and purification 56.7 mg (64%) of the title compound.

¹H-NMR (DMSO-d6): δ=0.90+0.94 (9H), 1.85 (1H), 2.08+2.18 (1H), 2.83-3.02 (2H), 2.92+3.17 (3H), 3.08-3.37 (5H), 3.97+3.99 (3H), 7.09 (1H), 7.99 (1H), 8.23 (1H), 8.45+8.47 (1H), 8.75-8.80 (1H), 12.86 (1H) ppm.

### Example 178

### {(7S)-4-[(6-Methoxy-1H-indazol-5-yl)amino]-5,6,7,8-tetrahydro[1]benzothieno[2,3-d]pyrimidin-7-yl}[3-(piperidin-1-yl)azetidin-1-yl]methanone

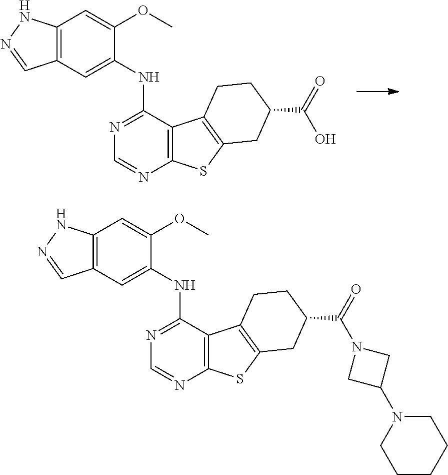

70 mg (177 μmol) (7S)-4-[(6-methoxy-1H-indazol-5-yl)amino]-5,6,7,8-tetrahydro[1]benzothieno[2,3-d]pyrimidine-7-carboxylic acid (prepared according to intermediate example 20a) were transformed in analogy to example 1 using 1-(azetidin-3-yl)piperidine to give after working up and purification 37.2 mg (39%) of the title compound.

¹H-NMR (DMSO-d6): δ=1.31-1.60 (7H), 1.81 (1H), 2.14 (1H), 2.18-2.29 (3H), 2.74-2.98 (3H), 3.01-3.25 (3H), 3.67 (1H), 3.88 (1H), 3.98 (3H), 4.05 (1H), 4.25 (1H), 7.09 (1H), 7.99 (1H), 8.21 (1H), 8.45 (1H), 8.77 (1H), 12.85 (1H) ppm.

### Example 179

### [(3R)-3-(Dimethylamino)pyrrolidin-1-yl]{(7S)-4-[(6-methoxy-1H-indazol-5-yl)amino]-5,6,7,8-tetrahydro[1]benzothieno[2,3-d]pyrimidin-7-yl}methanone

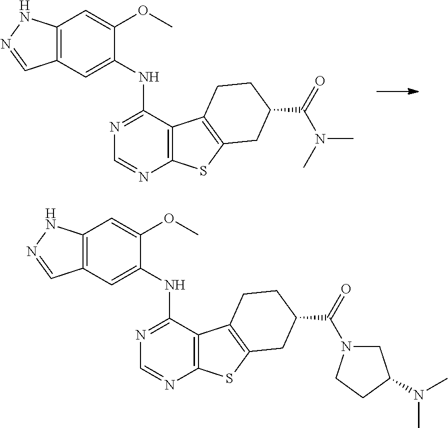 [SMILES: COc1cc2[nH]ncc2cc1Nc1ncnc2sc3c(c12)CC[C@H](C(=O)N(C)C)C3 ; COc1cc2[nH]ncc2cc1Nc1ncnc2sc3c(c12)CC[C@H](C(=O)N1CC[C@@H](N(C)C)C1)C3]

75 mg (190 μmol) (7S)-4-[(6-methoxy-1H-indazol-5-yl)amino]-5,6,7,8-tetrahydro[1]benzothieno[2,3-d]pyrimidine-7-carboxylic acid (prepared according to intermediate example 20a) were transformed in analogy to example 1 using (3R)—N,N-dimethylpyrrolidin-3-amine to give after working up and purification 25.6 mg (26%) of the title compound.

¹H-NMR (DMSO-d6): δ=1.58-1.88 (2H), 1.98-2.19 (2H), 2.16+2.17 (6H), 2.63+2.70 (1H), 2.89-3.66 (8H), 3.78+3.87 (1H), 3.98 (3H), 7.09 (1H), 7.99 (1H), 8.22 (1H), 8.45+8.46 (1H), 8.76+8.77 (1H), 12.84 (1H) ppm.

### Example 180

### tert-Butyl {3-[(5-{[(7S)-7-(dimethylcarbamoyl)-5,6,7,8-tetrahydro[1]benzothieno[2,3-d]pyrimidin-4-yl]amino}-1H-indazol-6-yl)oxy]propyl}carbamate

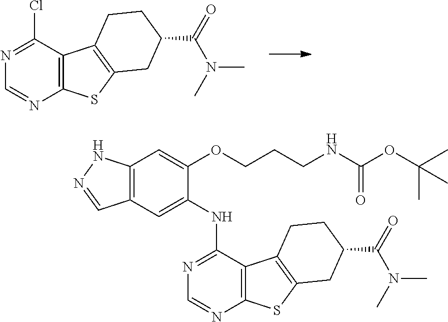 [SMILES: CN(C)C(=O)[C@H]1CCc2c(sc3ncnc(Cl)c23)C1 ; CN(C)C(=O)[C@H]1CCc2c(sc3ncnc(Nc4cc5cn[nH]c5cc4OCCCNC(=O)OC(C)(C)C)c23)C1]

100 mg (338 μmol) (7S)-4-chloro-N,N-dimethyl-5,6,7,8-tetrahydro[1]benzothieno[2,3-d]pyrimidine-7-carboxamide (prepared according to intermediate example 94a) were transformed in analogy to intermediate example 1b using tert-butyl {3-[(5-amino-1H-indazol-6-yl)oxy]propyl}carbamate (prepared according to intermediate example 180a) to give after working up and purification 19.1 mg (9%) of the title compound.

¹H-NMR (DMSO-d6): δ=1.36 (9H), 1.84 (1H), 1.99 (2H), 2.11 (1H), 2.89 (3H), 2.93 (2H), 3.12 (3H), 3.10-3.31 (5H), 4.17 (2H), 6.98 (1H), 7.06 (1H), 8.01 (1H), 8.26 (1H), 8.53 (1H), 9.01 (1H), 12.85 (1H) ppm.

### Example 180a

### tert-Butyl {3-[(5-amino-1H-indazol-6-yl)oxy]propyl}carbamate

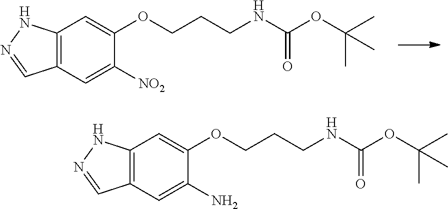 [SMILES: CC(C)(C)OC(=O)NCCCOc1cc2[nH]ncc2cc1[N+](=O)[O-] ; CC(C)(C)OC(=O)NCCCOc1cc2[nH]ncc2cc1N]

2.70 g (8.03 mmol) tert-butyl {3-[(5-nitro-1H-indazol-6-yl)oxy]propyl}carbamate (prepared according to intermediate example 180b) were transformed in analogy to intermediate example 94b to give after working up and purification 1.21 g (49%) of the title compound.

### Example 180b

### tert-Butyl {3-[(5-nitro-1H-indazol-6-yl)oxy]propyl}carbamate

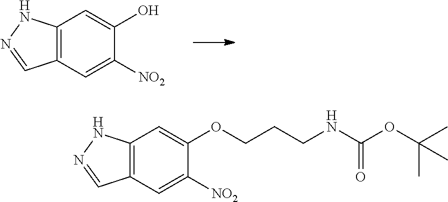 [SMILES: O=[N+]([O-])c1cc2cn[nH]c2cc1O ; CC(C)(C)OC(=O)NCCCOc1cc2[nH]ncc2cc1[N+](=O)[O-]]

2.00 g (11.2 mmol) 5-nitro-1H-indazol-6-ol (prepared according to intermediate example 94d) were transformed in analogy to intermediate example 94c using tert-butyl (3-hydroxypropyl)carbamate (CAS-No: 58885-58-8) to give after working up and purification 2.70 g (72%) of the title compound.

### Example 181

### (7S)-4-[(6-Bromo-1H-indazol-5-yl)amino]-N,N-dimethyl-5,6,7,8-tetrahydro[1]benzothieno[2,3-d]pyrimidine-7-carboxamide

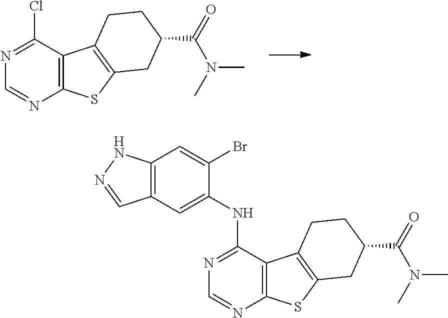 [SMILES: CN(C)C(=O)[C@H]1CCc2c(sc3ncnc(Cl)c23)C1 ; CN(C)C(=O)[C@H]1CCc2c(sc3ncnc(Nc4cc5cn[nH]c5cc4Br)c23)C1]

100 mg (338 μmol) (7S)-4-chloro-N,N-dimethyl-5,6,7,8-tetrahydro[1]benzothieno[2,3-d]pyrimidine-7-carboxamide (prepared according to intermediate example 94a) were transformed in analogy to intermediate example 1b using 6-bromo-1H-indazol-5-amine (prepared according to intermediate example 181a) to give after working up and purification 39.6 mg (24%) of the title compound.

¹H-NMR (DMSO-d6): δ=1.78 (1H), 2.08 (1H), 2.87 (3H), 2.92 (2H), 3.05-3.27 (3H), 3.09 (3H), 7.92 (1H), 8.05 (1H), 8.12 (1H), 8.22 (1H), 8.28 (1H), 13.20 (1H) ppm.

### Example 181a

### 6-Bromo-1H-indazol-5-amine

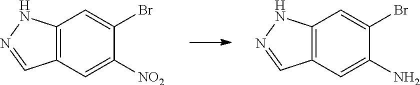 [SMILES: O=[N+]([O-])c1cc2cn[nH]c2cc1Br ; Nc1cc2cn[nH]c2cc1Br]

10.0 g (41.3 mmol) 6-bromo-5-nitro-1H-indazole (prepared according to intermediate example 181b) were transformed in analogy to intermediate example 128a to give after working up and purification 8.44 g (92%) of the title compound.

### Example 181b

### 6-Bromo-5-nitro-1H-indazole

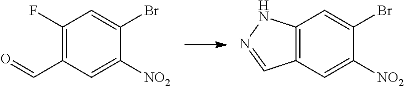 [SMILES: O=Cc1cc([N+](=O)[O-])c(Br)cc1F ; O=[N+]([O-])c1cc2cn[nH]c2cc1Br]

25.0 g (100.8 mmol) 4-bromo-2-fluoro-5-nitrobenzaldehyde (CAS-No: 679839-39-5) were transformed in analogy to intermediate example 94d to give after working up and purification 20.8 g (86%) of the title compound.

### Example 182

### (7S)-4-[(6-Chloro-1H-indazol-5-yl)amino]-N,N-dimethyl-5,6,7,8-tetrahydro[1]benzothieno[2,3-d]pyrimidine-7-carboxamide

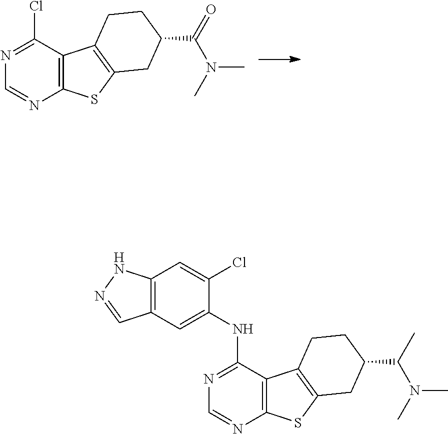 [SMILES: CN(C)C(=O)[C@H]1CCc2c(sc3ncnc(Cl)c23)C1 ; CC([C@H]1CCc2c(sc3ncnc(Nc4cc5cn[nH]c5cc4Cl)c23)C1)N(C)C]

100 mg (338 μmol) (7S)-4-chloro-N,N-dimethyl-5,6,7,8-tetrahydro[1]benzothieno[2,3-d]pyrimidine-7-carboxamide (prepared according to intermediate example 94a) were transformed in analogy to intermediate example 1b using 6-chloro-1H-indazol-5-amine (CAS-No: 221681-75-0) to give after working up and purification 49.2 mg (33%) of the title compound.

¹H-NMR (DMSO-d6): δ=1.78 (1H), 2.07 (1H), 2.87 (3H), 2.92 (2H), 3.12-3.30 (3H), 3.09 (3H), 7.76 (1H), 8.11 (1H), 8.13 (1H), 8.24 (1H), 8.27 (1H), 13.20 (1H) ppm.

### Example 183

### [(2R,6S)-2,6-Dimethylmorpholin-4-yl][(7S)-4-{[6-(pyrrolidin-1-yl)-1H-indazol-5-yl]amino}-5,6,7,8-tetrahydro[1]benzothieno[2,3-d]pyrimidin-7-yl]methanone

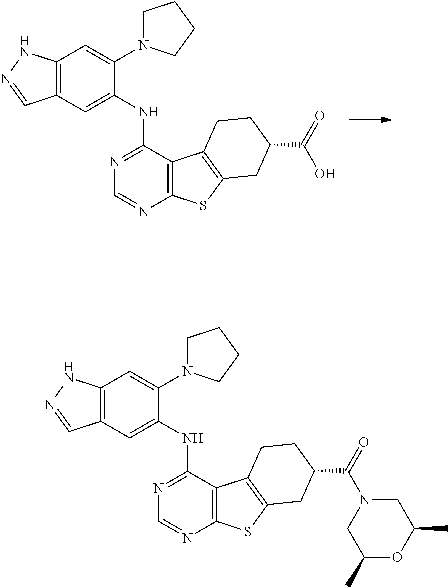 [SMILES: O=C(O)[C@H]1CCc2c(sc3ncnc(Nc4cc5cn[nH]c5cc4N4CCCC4)c23)C1 ; C[C@@H]1CN(C(=O)[C@H]2CCc3c(sc4ncnc(Nc5cc6cn[nH]c6cc5N5CCCC5)c34)C2)C[C@H](C)O1]

50 mg (115 μmol) (7S)-4-{[6-(pyrrolidin-1-yl)-1H-indazol-5-yl]amino}-5,6,7,8-tetrahydro[1]benzothieno[2,3-d]pyrimidine-7-carboxylic acid (prepared according to intermediate example 183a) were transformed in analogy to example 1 using (2R,6S)-2,6-dimethylmorpholine to give after working up and purification 16.6 mg (26%) of the title compound.

¹H-NMR (DMSO-d6): δ=1.11+1.13 (6H), 1.81-2.00 (5H), 2.06 (1H), 2.28 (1H), 2.78 (1H), 2.89-3.03 (2H), 3.08-3.16 (4H), 3.19-3.31 (3H), 3.44 (1H), 3.54 (1H), 3.99 (1H), 4.32 (1H), 7.31+7.33 (1H), 8.00 (1H), 8.46+8.47 (1H), 8.72+8.77 (1H), 8.90+8.96 (1H), 12.82 (1H) ppm.

### Example 183a

### (7S)-4-{[6-(Pyrrolidin-1-yl)-1H-indazol-5-yl]amino}-5,6,7,8-tetrahydro[1]benzothieno[2,3-d]pyrimidine-7-carboxylic acid

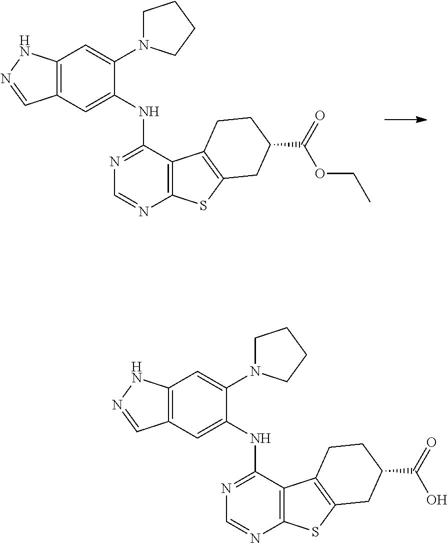 [SMILES: CCOC(=O)[C@H]1CCc2c(sc3ncnc(Nc4cc5cn[nH]c5cc4N4CCCC4)c23)C1 ; O=C(O)[C@H]1CCc2c(sc3ncnc(Nc4cc5cn[nH]c5cc4N4CCCC4)c23)C1]

495 mg (1.07 mmol) ethyl (7S)-4-{[6-(pyrrolidin-1-yl)-1H-indazol-5-yl]amino}-5,6,7,8-tetrahydro[1]benzothieno[2,3-d]pyrimidine-7-carboxylate (prepared according to intermediate example 183b) were transformed in analogy to intermediate example 1a to give after working up and purification 409 mg (88%) of the title compound.

### Example 183b

### Ethyl (7S)-4-{[6-(pyrrolidin-1-yl)-1H-indazol-5-yl]amino}-5,6,7,8-tetrahydro[1]benzothieno[2,3-d]pyrimidine-7-carboxylate

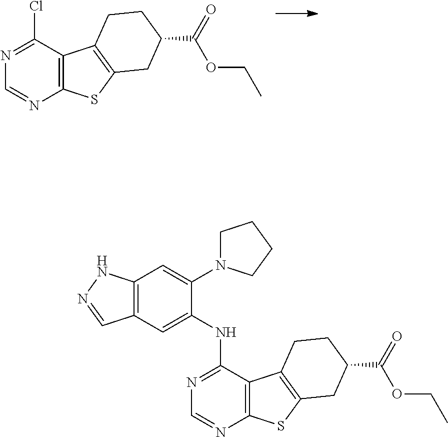 [SMILES: CCOC(=O)[C@H]1CCc2c(sc3ncnc(Cl)c23)C1 ; CCOC(=O)[C@H]1CCc2c(sc3ncnc(Nc4cc5cn[nH]c5cc4N4CCCC4)c23)C1]

1.12 g (3.77 mmol) ethyl (7S)-4-chloro-5,6,7,8-tetrahydro[1]benzothieno[2,3-d]pyrimidine-7-carboxylate (prepared according to intermediate example 1c) were transformed in analogy to intermediate example 1b using 6-(pyrrolidin-1-yl)-1H-indazol-5-amine (prepared according to intermediate example 103a) to give after working up and purification 506 mg (29%) of the title compound.

### Example 184

### [(3R)-3-Methylmorpholin-4-yl][(7S)-4-{[6-(pyrrolidin-1-yl)-1H-indazol-5-yl]amino}-5,6,7,8-tetrahydro[1]benzothieno[2,3-d]pyrimidin-7-yl]methanone

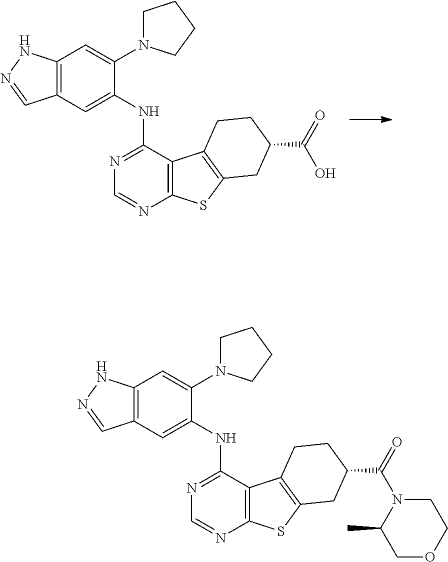 [SMILES: O=C(O)[C@H]1CCc2c(sc3ncnc(Nc4cc5cn[nH]c5cc4N4CCCC4)c23)C1 ; C[C@@H]1COCCN1C(=O)[C@H]1CCc2c(sc3ncnc(Nc4cc5cn[nH]c5cc4N4CCCC4)c23)C1]

50 mg (115 μmol) (7S)-4-{[6-(pyrrolidin-1-yl)-1H-indazol-5-yl]amino}-5,6,7,8-tetrahydro[1]benzothieno[2,3-d]pyrimidine-7-carboxylic acid (prepared according to intermediate example 183a) were transformed in analogy to example 1 using (3R)-3-methylmorpholine to give after working up and purification 13.7 mg (22%) of the title compound.

¹H-NMR (DMSO-d6): δ=1.16+1.32 (3H), 1.88-2.11 (6H), 2.86-4.46 (16H), 7.32 (1H), 8.00 (1H), 8.47 (1H), 8.73+8.77 (1H), 8.92+8.97 (1H), 12.82 (1H) ppm.

### Example 185

### [(3S)-3-Methylmorpholin-4-yl][(7S)-4-{[6-(pyrrolidin-1-yl)-1H-indazol-5-yl]amino}-5,6,7,8-tetrahydro[1]benzothieno[2,3-d]pyrimidin-7-yl]methanone

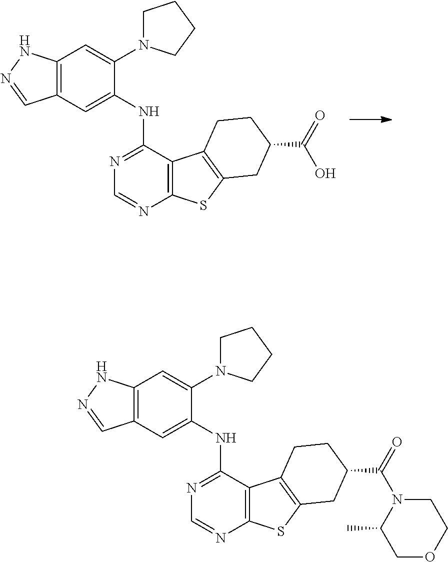 [SMILES: O=C(O)[C@H]1CCc2c(sc3ncnc(Nc4cc5cn[nH]c5cc4N4CCCC4)c23)C1 ; C[C@H]1COCCN1C(=O)[C@H]1CCc2c(sc3ncnc(Nc4cc5cn[nH]c5cc4N4CCCC4)c23)C1]

50 mg (115 μmol) (7S)-4-{[6-(pyrrolidin-1-yl)-1H-indazol-5-yl]amino}-5,6,7,8-tetrahydro[1]benzothieno[2,3-d]pyrimidine-7-carboxylic acid (prepared according to intermediate example 183a) were transformed in analogy to example 1 using (3S)-3-methylmorpholine to give after working up and purification 17.6 mg (28%) of the title compound.

¹H-NMR (DMSO-d6): δ=1.17+1.30 (3H), 1.77-2.13 (6H), 2.85-4.47 (16H), 7.31 (1H), 8.00 (1H), 8.46 (1H), 8.72 (1H), 8.89 (1H), 12.81 (1H) ppm.

### Example 186

### (7S)—N,N-Dimethyl-4-{[6-(methylsulfonyl)-1H-indazol-5-yl]amino}-5,6,7,8-tetrahydro[1]benzothieno[2,3-d]pyrimidine-7-carboxamide

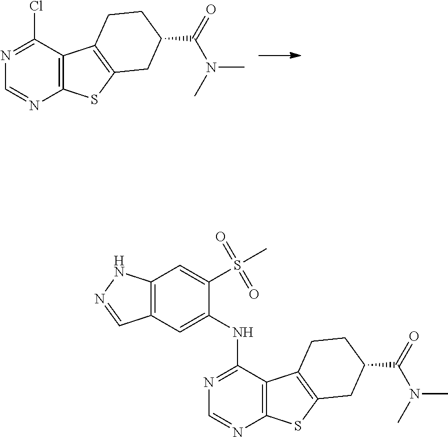 [SMILES: CN(C)C(=O)[C@H]1CCc2c(sc3ncnc(Cl)c23)C1 ; CN(C)C(=O)[C@H]1CCc2c(sc3ncnc(Nc4cc5cn[nH]c5cc4S(C)(=O)=O)c23)C1]

50 mg (169 μmol) (7S)-4-chloro-N,N-dimethyl-5,6,7,8-tetrahydro[1]benzothieno[2,3-d]pyrimidine-7-carboxamide (prepared according to intermediate example 94a) were transformed in analogy to intermediate example 1b using 6-(methylsulfonyl)-1H-indazol-5-amine (prepared according to intermediate example 186a) to give after working up and purification 11.2 mg (13%) of the title compound.

¹H-NMR (DMSO-d6): δ=1.79 (1H), 2.09 (1H), 2.88 (3H), 2.90-3.03 (2H), 3.07-3.30 (3H), 3.11 (3H), 3.19 (3H), 8.21 (1H), 8.30 (1H), 8.31 (1H), 8.35 (1H), 8.47 (1H), 13.70 (1H) ppm.

### Example 186a

### 6-(Methylsulfonyl)-1H-indazol-5-amine

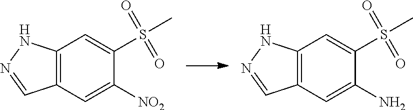 [SMILES: CS(=O)(=O)c1cc2[nH]ncc2cc1[N+](=O)[O-] ; CS(=O)(=O)c1cc2[nH]ncc2cc1N]

180 mg (746 μmol) 6-(methylsulfonyl)-5-nitro-1H-indazole (prepared according to intermediate example 186b) were transformed in analogy to intermediate example 94b to give after working up and purification 128 mg (81%) of the title compound.

### Example 186b

### 6-(Methylsulfonyl)-5-nitro-1H-indazole

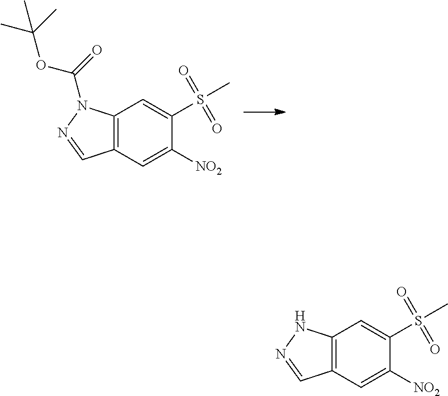 [SMILES: CC(C)(C)OC(=O)n1ncc2cc([N+](=O)[O-])c(S(C)(=O)=O)cc21 ; CS(=O)(=O)c1cc2[nH]ncc2cc1[N+](=O)[O-]]

A mixture comprising 289.6 mg (848 μmol) tert-butyl 6-(methylsulfonyl)-5-nitro-1H-indazole-1-carboxylate (prepared according to intermediate example 186c), 11.5 mL tetrahydrofurane, 5.5 mL ethanol and 582 μL hydrochloric acid (4M in dioxane) was heated at 100° C. under microwave irradiation for two hours. Water and saturated sodium hydrogencarbonate solution were added, the organic solvents were removed, the precipitate collected, washed with water and dried to give 184 mg (90%) of the title compound.

### Example 186c

### tert-Butyl 6-(methylsulfonyl)-5-nitro-1H-indazole-1-carboxylate

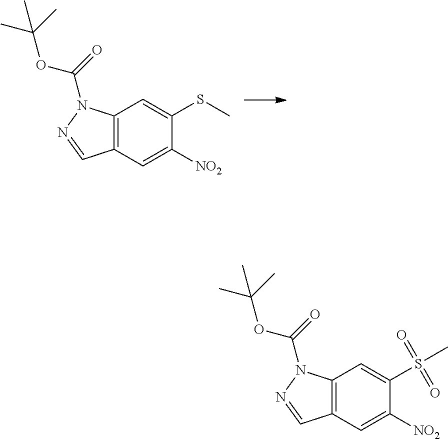 [SMILES: CSc1cc2c(cnn2C(=O)OC(C)(C)C)cc1[N+](=O)[O-] ; CC(C)(C)OC(=O)n1ncc2cc([N+](=O)[O-])c(S(C)(=O)=O)cc21]

A mixture comprising 250 mg (808 μmol) tert-butyl 6-(methylsulfanyl)-5-nitro-1H-indazole-1-carboxylate (prepared according to intermediate example 186d), 10 mL dichloromethane and 465 mg 3-chlorobenzenecarboperoxoic acid (75%) was stirred at 23° C. overnight. Saturated sodium hydrogencarbonate solution was added, the organic layer was washed with water, brine and dried over sodium sulphate. After filtration and removal of the solvent, the residue was purified by chromatography to give 257 mg (93%) of the title compound.

### Example 186d

### tert-Butyl 6-(methylsulfanyl)-5-nitro-1H-indazole-1-carboxylate

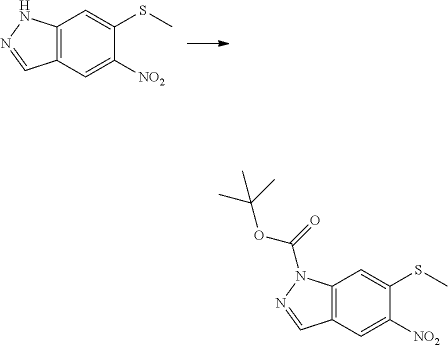 [SMILES: CSc1cc2[nH]ncc2cc1[N+](=O)[O-] ; CSc1cc2c(cnn2C(=O)OC(C)(C)C)cc1[N+](=O)[O-]]

A mixture comprising 500 mg (2.39 mmol) 6-(methylsulfanyl)-5-nitro-1H-indazole prepared according to intermediate example 106b), 10 mL tetrahydrofurane, 8.8 mg N,N-dimethylpyridin-4-amine and 1.13 mL di-tert-butyl dicarbonate was stirred at 23° C. overnight. Water and ethyl acetate were added, the organic layer was washed with saturated sodium hydrogencarbonate, brine and dried over sodium sulphate. After filtration and removal of the solvent, the residue was purified by rescrystallisation to give 614 mg (83%) of the title compound.

### Example 187

### [(7S)-4-{[6-(Dimethylamino)-1H-indazol-5-yl]amino}-5,6,7,8-tetrahydro[1]benzothieno[2,3-d]pyrimidin-7-yl][(3R)-3-methylmorpholin-4-yl]methanone

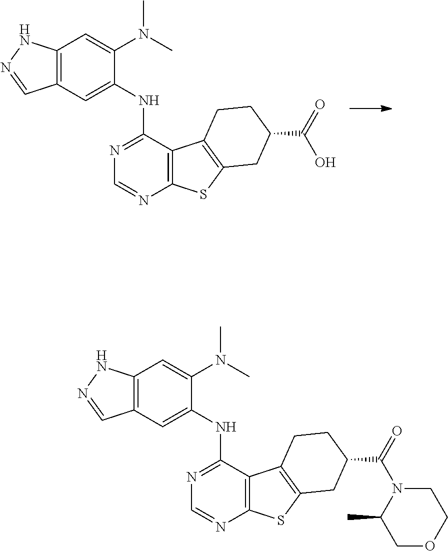 [SMILES: CN(C)c1cc2[nH]ncc2cc1Nc1ncnc2sc3c(c12)CC[C@H](C(=O)O)C3 ; C[C@@H]1COCCN1C(=O)[C@H]1CCc2c(sc3ncnc(Nc4cc5cn[nH]c5cc4N(C)C)c23)C1]

50 mg (122 μmol) (7S)-4-{[6-(dimethylamino)-1H-indazol-5-yl]amino}-5,6,7,8-tetrahydro[1]benzothieno[2,3-d]pyrimidine-7-carboxylic acid (prepared according to intermediate example 187a) were transformed in analogy to example 1 using (3R)-3-methylmorpholine to give after working up and purification 36.8 mg (60%) of the title compound.

¹H-NMR (DMSO-d6): δ=1.16+1.33 (3H), 1.96 (1H), 2.14 (1H), 2.74 (6H), 2.85-4.48 (12H), 7.44 (1H), 8.05 (1H), 8.53 (1H), 8.99 (1H), 9.15 (1H), 12.91 (1H) ppm.

### Example 187a

### (7S)-4-{[6-(Dimethylamino)-1H-indazol-5-yl]amino}-5,6,7,8-tetrahydro[1]benzothieno[2,3-d]pyrimidine-7-carboxylic acid

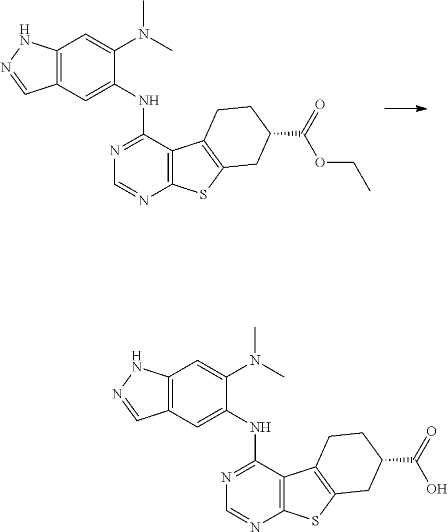 [SMILES: CCOC(=O)[C@H]1CCc2c(sc3ncnc(Nc4cc5cn[nH]c5cc4N(C)C)c23)C1 ; CN(C)c1cc2[nH]ncc2cc1Nc1ncnc2sc3c(c12)CC[C@H](C(=O)O)C3]

547 mg (1.25 mmol) ethyl (7S)-4-{[6-(dimethylamino)-1H-indazol-5-yl]amino}-5,6,7,8-tetrahydro[1]benzothieno[2,3-d]pyrimidine-7-carboxylate (prepared according to intermediate example 187b) were transformed in analogy to intermediate example 1a to give after working up and purification 453 mg (89%) of the title compound.

### Example 187b

### Ethyl (7S)-4-{[6-(dimethylamino)-1H-indazol-5-yl]amino}-5,6,7,8-tetrahydro[1]benzothieno[2,3-d]pyrimidine-7-carboxylate

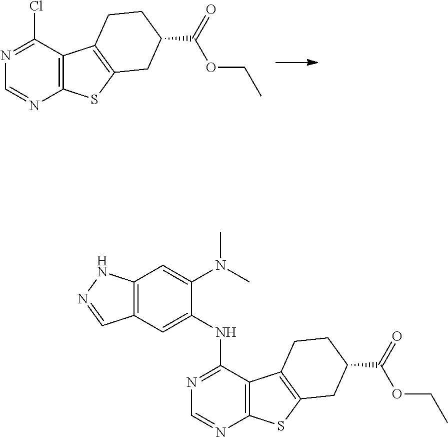 [SMILES: CCOC(=O)[C@H]1CCc2c(sc3ncnc(Cl)c23)C1 ; CCOC(=O)[C@H]1CCc2c(sc3ncnc(Nc4cc5cn[nH]c5cc4N(C)C)c23)C1]

1.50 g (5.05 mmol) ethyl (7S)-4-chloro-5,6,7,8-tetrahydro[1]benzothieno[2,3-d]pyrimidine-7-carboxylate (prepared according to intermediate example 1c) were transformed in analogy to intermediate example 1b using N⁶,N⁶-dimethyl-1H-indazole-5,6-diamine (prepared according to intermediate example 98a) to give after working up and purification 556 mg (25%) of the title compound.

### Example 188

### [(7S)-4-{[6-(Dimethylamino)-1H-indazol-5-yl]amino}-5,6,7,8-tetrahydro[1]benzothieno[2,3-d]pyrimidin-7-yl][(3S)-3-methylmorpholin-4-yl]methanone

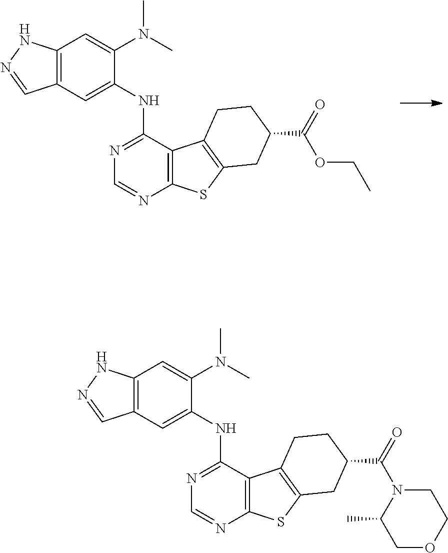 [SMILES: CCOC(=O)[C@H]1CCc2c(sc3ncnc(Nc4cc5cn[nH]c5cc4N(C)C)c23)C1 ; C[C@H]1COCCN1C(=O)[C@H]1CCc2c(sc3ncnc(Nc4cc5cn[nH]c5cc4N(C)C)c23)C1]

50 mg (122 μmol) (7S)-4-{[6-(dimethylamino)-1H-indazol-5-yl]amino}-5,6,7,8-tetrahydro[1]benzothieno[2,3-d]pyrimidine-7-carboxylic acid (prepared according to intermediate example 187a) were transformed in analogy to example 1 using (3S)-3-methylmorpholine to give after working up and purification 35.2 mg (56%) of the title compound.

¹H-NMR (DMSO-d6): δ=1.17+1.31 (3H), 1.86 (1H), 2.15 (1H), 2.74 (6H), 2.87-4.49 (12H), 7.44 (1H), 8.05 (1H), 8.53 (1H), 9.01 (1H), 9.12+9.16 (1H), 12.91 (1H) ppm.

### Example 189

### [(7S)-4-{[6-(Dimethylamino)-1H-indazol-5-yl]amino}-5,6,7,8-tetrahydro[1]benzothieno[2,3-d]pyrimidin-7-yl][(2R,6S)-2,6-dimethylmorpholin-4-yl]methanone

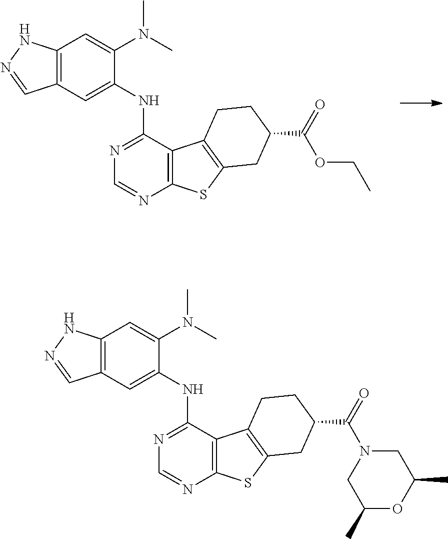 [SMILES: CCOC(=O)[C@H]1CCc2c(sc3ncnc(Nc4cc5cn[nH]c5cc4N(C)C)c23)C1 ; C[C@@H]1CN(C(=O)[C@H]2CCc3c(sc4ncnc(Nc5cc6cn[nH]c6cc5N(C)C)c34)C2)C[C@H](C)O1]

50 mg (122 μmol) (7S)-4-{[6-(dimethylamino)-1H-indazol-5-yl]amino}-5,6,7,8-tetrahydro[1]benzothieno[2,3-d]pyrimidine-7-carboxylic acid (prepared according to intermediate example 187a) were transformed in analogy to example 1 using (2R,6S)-2,6-dimethylmorpholine to give after working up and purification 27.0 mg (41%) of the title compound.

¹H-NMR (DMSO-d6): δ=1.12+1.13 (6H), 1.91 (1H), 2.14 (1H), 2.28 (1H), 2.74 (6H), 2.78 (1H), 2.85-3.03 (2H), 3.16-3.31 (3H), 3.45 (1H), 3.54 (1H), 4.02 (1H), 4.33 (1H), 7.44 (1H), 8.05 (1H), 8.53 (1H), 8.98 (1H), 9.13+9.15 (1H), 12.90 (1H) ppm.

### Example 190

### [(3R)-3-(Dimethylamino)pyrrolidin-1-yl]{(7S)-4-[(6-ethoxy-1H-indazol-5-yl)amino]-5,6,7,8-tetrahydro[1]benzothieno[2,3-d]pyrimidin-7-yl}methanone

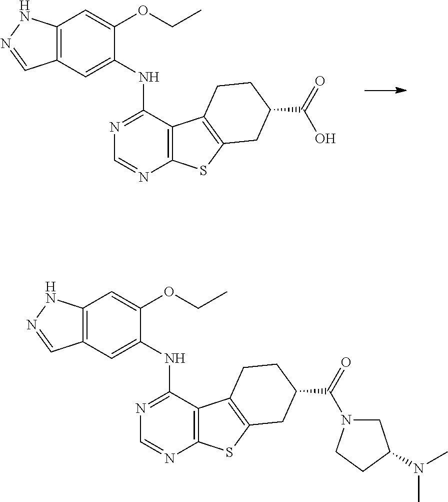 [SMILES: CCOc1cc2[nH]ncc2cc1Nc1ncnc2sc3c(c12)CC[C@H](C(=O)O)C3 ; CCOc1cc2[nH]ncc2cc1Nc1ncnc2sc3c(c12)CC[C@H](C(=O)N1CC[C@@H](N(C)C)C1)C3]

75 mg (183 μmol) (7S)-4-[(6-ethoxy-1H-indazol-5-yl)amino]-5,6,7,8-tetrahydro[1]benzothieno[2,3-d]pyrimidine-7-carboxylic acid (prepared according to intermediate example 65a) were transformed in analogy to example 1 using (3R)—N,N-dimethylpyrrolidin-3-amine to give after working up and purification 29.3 mg (30%) of the title compound.

¹H-NMR (DMSO-d6): δ=1.47 (3H), 1.65+1.75 (1H), 1.85 (1H), 1.98-2.14 (2H), 2.16+2.17 (6H), 2.63+2.70 (1H), 2.90-3.64 (8H), 3.78+3.86 (1H), 4.21 (2H), 7.06 (1H), 7.99 (1H), 8.35+8.36 (1H), 8.52 (1H), 9.01 (1H), 12.81 (1H) ppm.

### Example 191

### (7S)—N,N-Dimethyl-4-{[6-(trifluoromethoxy)-1H-indazol-5-yl]amino}-5,6,7,8-tetrahydro[1]benzothieno[2,3-d]pyrimidine-7-carboxamide

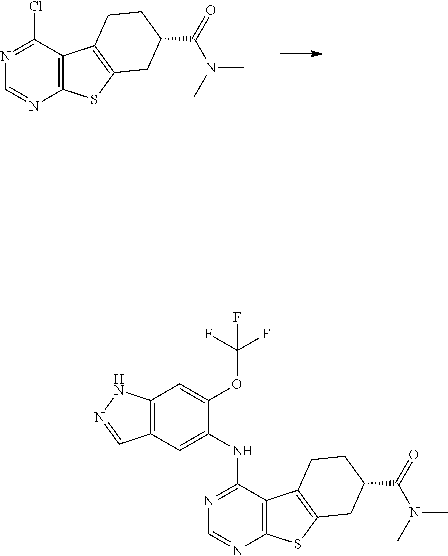 [SMILES: CN(C)C(=O)[C@H]1CCc2c(sc3ncnc(Cl)c23)C1 ; CN(C)C(=O)[C@H]1CCc2c(sc3ncnc(Nc4cc5cn[nH]c5cc4OC(F)(F)F)c23)C1]

200 mg (676 μmol) (7S)-4-chloro-N,N-dimethyl-5,6,7,8-tetrahydro[1]benzothieno[2,3-d]pyrimidine-7-carboxamide (prepared according to intermediate example 94a) were transformed in analogy to intermediate example 1b using 6-(trifluoromethoxy)-1H-indazol-5-amine (prepared according to intermediate example 191a) to give after working up and purification 201 mg (62%) of the title compound.

¹H-NMR (DMSO-d6): δ=1.79 (1H), 2.07 (1H), 2.87 (3H), 2.93 (2H), 3.05-3.26 (3H), 3.09 (3H), 7.60 (1H), 8.16 (1H), 8.18 (1H), 8.21 (1H), 8.26 (1H), 13.26 (1H) ppm.

### Example 191a

### 6-(Trifluoromethoxy)-1H-indazol-5-amine

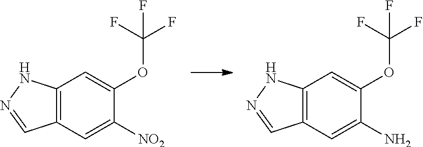 [SMILES: O=[N+]([O-])c1cc2cn[nH]c2cc1OC(F)(F)F ; Nc1cc2cn[nH]c2cc1OC(F)(F)F]

1.00 g (4.05 mmol) 5-nitro-6-(trifluoromethoxy)-1H-indazole (prepared according to intermediate example 98b) were transformed in analogy to intermediate example 94b to give after working up and purification 878 mg (100%) of the title compound.

### Example 192

### (7S)—N,N-Dimethyl-4-{[6-(propylsulfanyl)-1H-indazol-5-yl]amino}-5,6,7,8-tetrahydro[1]benzothieno[2,3-d]pyrimidine-7-carboxamide

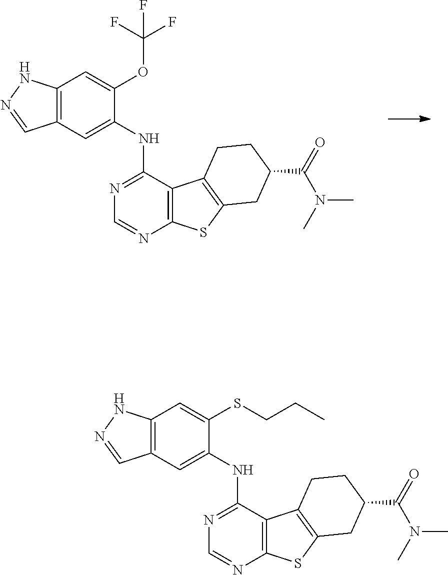 [SMILES: CN(C)C(=O)[C@H]1CCc2c(sc3ncnc(Nc4cc5cn[nH]c5cc4OC(F)(F)F)c23)C1 ; CCCSc1cc2[nH]ncc2cc1Nc1ncnc2sc3c(c12)CC[C@H](C(=O)N(C)C)C3]

67 mg (141 μmol) (7S)—N,N-Dimethyl-4-{[6-(trifluoromethoxy)-1H-indazol-5-yl]amino}-5,6,7,8-tetrahydro[1]benzothieno[2,3-d]pyrimidine-7-carboxamide (prepared according to example 191) were transformed in analogy to intermediate example 106b using sodium propane-1-thiolate to give after working up and purification 14.5 mg (21%) of the title compound.

¹H-NMR (DMSO-d6): δ=0.93 (3H), 1.56 (2H), 1.80 (1H), 2.09 (1H), 2.84-3.00 (4H), 2.87 (3H), 3.09 (3H), 3.20 (2H), 3.35 (1H), 7.57 (1H), 8.06 (1H), 8.22 (1H), 8.29 (1H), 8.32 (1H), 12.99 (1H) ppm.

### Example 193

### (7S)—N,N-Dimethyl-4-({6-[2-(2-oxopyrrolidin-1-yl)ethoxy]-1H-indazol-5-yl}amino)-5,6,7,8-tetrahydro[1]benzothieno[2,3-d]pyrimidine-7-carboxamide

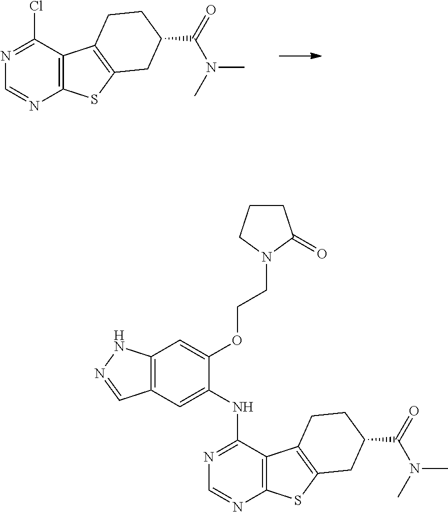 [SMILES: CN(C)C(=O)[C@H]1CCc2c(sc3ncnc(Cl)c23)C1 ; CN(C)C(=O)[C@H]1CCc2c(sc3ncnc(Nc4cc5cn[nH]c5cc4OCCN4CCCC4=O)c23)C1]

50 mg (169 μmol) (7S)-4-chloro-N,N-dimethyl-5,6,7,8-tetrahydro[1]benzothieno[2,3-d]pyrimidine-7-carboxamide (prepared according to intermediate example 94a) were transformed in analogy to intermediate example 1b using 1-{2-[(5-amino-1H-indazol-6-yl)oxy]ethyl}pyrrolidin-2-one (prepared according to intermediate example 193a) to give after working up and purification 47.1 mg (51%) of the title compound.

¹H-NMR (DMSO-d6): δ=1.73-1.89 (3H), 2.09-2.18 (3H), 2.89 (3H), 2.94 (2H), 3.12 (3H), 3.16-3.38 (5H), 3.58 (1H), 3.68 (1H), 4.29 (2H), 7.15 (1H), 8.01 (1H), 8.14 (1H), 8.44 (1H), 8.68 (1H), 12.89 (1H) ppm.

### Example 193a

### 1-{2-[(5-Amino-1H-indazol-6-yl)oxy]ethyl}pyrrolidin-2-one carboxamide

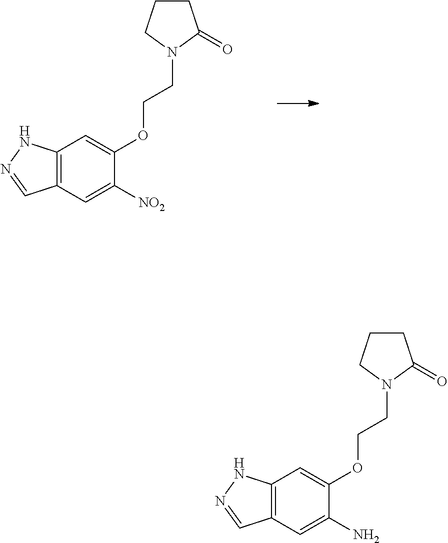 [SMILES: O=C1CCCN1CCOc1cc2[nH]ncc2cc1[N+](=O)[O-] ; Nc1cc2cn[nH]c2cc1OCCN1CCCC1=O]

719 mg (2.48 mmol) 1-{2-[(5-nitro-1H-indazol-6-yl)oxy]ethyl}pyrrolidin-2-one (prepared according to intermediate example 193b) were transformed in analogy to intermediate example 94b to give after working up and purification 265 mg (41%) of the title compound.

### Example 193b

### 1-{2-[(5-Nitro-1H-indazol-6-yl)oxy]ethyl}pyrrolidin-2-one

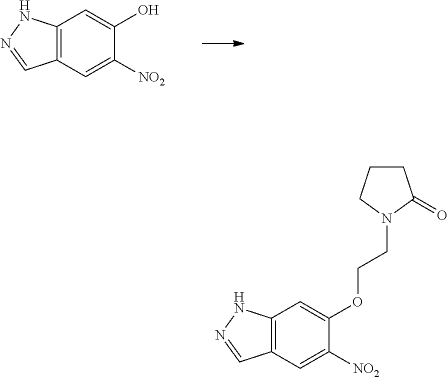 [SMILES: O=[N+]([O-])c1cc2cn[nH]c2cc1O ; O=C1CCCN1CCOc1cc2[nH]ncc2cc1[N+](=O)[O-]]

500 mg (2.79 mmol) 5-nitro-1H-indazol-6-ol (prepared according to intermediate example 94d) were transformed in analogy to intermediate example 94c using 1-(2-hydroxyethyl)pyrrolidin-2-one to give after working up and purification 271 mg (33%) of the title compound.

### Example 194

### (7S)—N,N-Dimethyl-4-({6-[2-(2-oxo-1, 3-oxazolidin-3-yl)ethoxy]-1H-indazol-5-yl}amino)-5,6,7,8-tetrahydro[1]benzothieno[2,3-d]pyrimidine-7-carboxamide

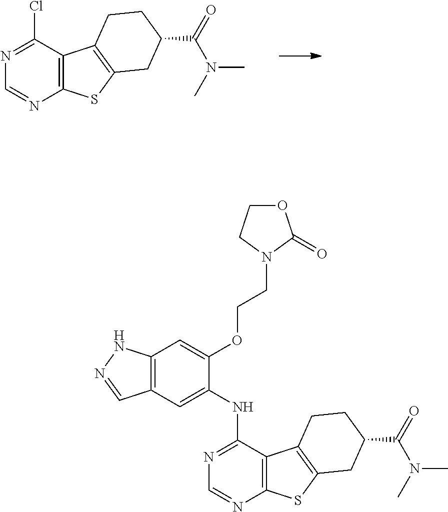 [SMILES: CN(C)C(=O)[C@H]1CCc2c(sc3ncnc(Cl)c23)C1 ; CN(C)C(=O)[C@H]1CCc2c(sc3ncnc(Nc4cc5cn[nH]c5cc4OCCN4CCOC4=O)c23)C1]

50 mg (169 μmol) (7S)-4-chloro-N,N-dimethyl-5,6,7,8-tetrahydro[1]benzothieno[2,3-d]pyrimidine-7-carboxamide (prepared according to intermediate example 94a) were transformed in analogy to intermediate example 1b using 3-{2-[(5-amino-1H-indazol-6-yl)oxy]ethyl}-1,3-oxazolidin-2-one (prepared according to intermediate example 194a) to give after working up and purification 27.1 mg (29%) of the title compound.

¹H-NMR (DMSO-d6): δ=1.81 (1H), 2.16 (1H), 2.89 (3H), 2.94 (2H), 3.12 (3H), 3.13-3.39 (3H), 3.56 (2H), 3.64 (2H), 4.19 (2H), 4.35 (2H), 7.17 (1H), 8.01 (1H), 8.13 (1H), 8.47 (1H), 8.76 (1H), 12.90 (1H) ppm.

### Example 194a

### 3-{2-[(5-Amino-1H-indazol-6-yl)oxy]ethyl}-1,3-oxazolidin-2-one

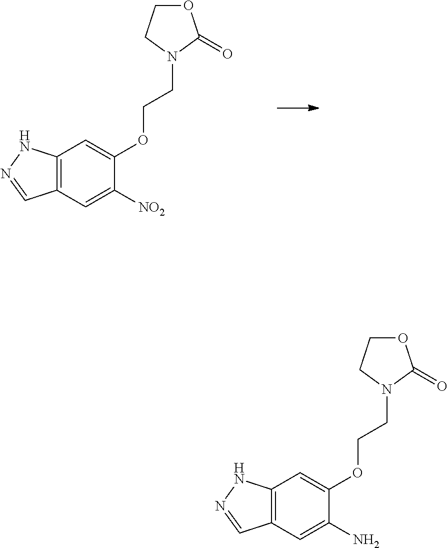 [SMILES: O=C1OCCN1CCOc1cc2[nH]ncc2cc1[N+](=O)[O-] ; Nc1cc2cn[nH]c2cc1OCCN1CCOC1=O]

502 mg (1.72 mmol) 3-{2-[(5-nitro-1H-indazol-6-yl)oxy]ethyl}-1,3-oxazolidin-2-one (prepared according to intermediate example 194b) were transformed in analogy to intermediate example 94b to give after working up and purification 295 mg (65%) of the title compound.

### Example 194b

### 3-{2-[(5-Nitro-1H-indazol-6-yl)oxy]ethyl}-1,3-oxazolidin-2-one

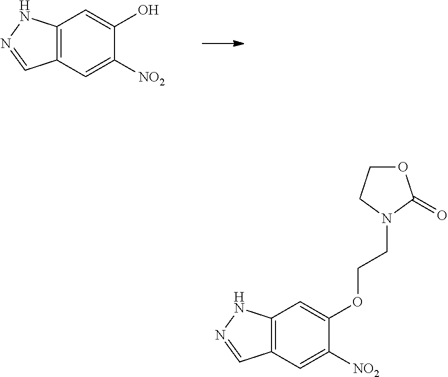 [SMILES: O=[N+]([O-])c1cc2cn[nH]c2cc1O ; O=C1OCCN1CCOc1cc2[nH]ncc2cc1[N+](=O)[O-]]

500 mg (2.79 mmol) 5-nitro-1H-indazol-6-ol (prepared according to intermediate example 94d) were transformed in analogy to intermediate example 94c using 3-(2-hydroxyethyl)-1,3-oxazolidin-2-one to give after working up and purification 509 mg (62%) of the title compound.

### Example 195

### Azetidin-1-yl[(7S)-4-{[6-(pyrrolidin-1-yl)-1H-indazol-5-yl]amino}-5,6,7,8-tetrahydro[1]benzothieno[2,3-d]pyrimidin-7-yl]methanone

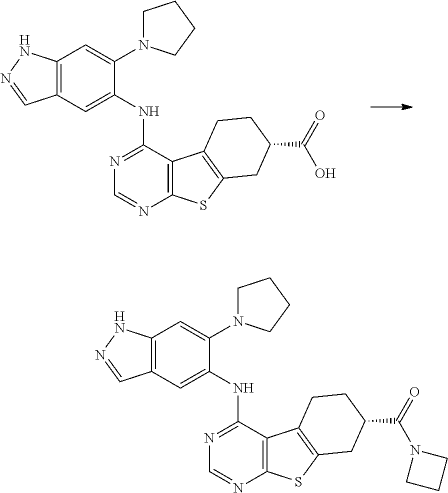 [SMILES: O=C(O)[C@H]1CCc2c(sc3ncnc(Nc4cc5cn[nH]c5cc4N4CCCC4)c23)C1 ; O=C([C@H]1CCc2c(sc3ncnc(Nc4cc5cn[nH]c5cc4N4CCCC4)c23)C1)N1CCC1]

70 mg (141 μmol) (7S)-4-{[6-(pyrrolidin-1-yl)-1H-indazol-5-yl]amino}-5,6,7,8-tetrahydro[1]benzothieno[2,3-d]pyrimidine-7-carboxylic acid (prepared according to intermediate example 183a) were transformed in analogy to example 1 using azetidine to give after working up and purification 44.3 mg (55%) of the title compound.

¹H-NMR (DMSO-d6): δ=1.83 (1H), 1.93 (4H), 2.06 (1H), 2.21 (2H), 2.78 (1H), 2.88 (2H), 3.02-3.17 (5H), 3.24 (1H), 3.88 (2H), 4.23 (2H), 7.30 (1H), 7.99 (1H), 8.44 (1H), 8.72 (1H), 8.90 (1H), 12.80 (1H) ppm.

### Example 196

### Azetidin-1-yl[(7S)-4-{[6-(dimethylamino)-1H-indazol-5-yl]amino}-5,6,7,8-tetrahydro[1]benzothieno[2,3-d]pyrimidin-7-yl]methanone

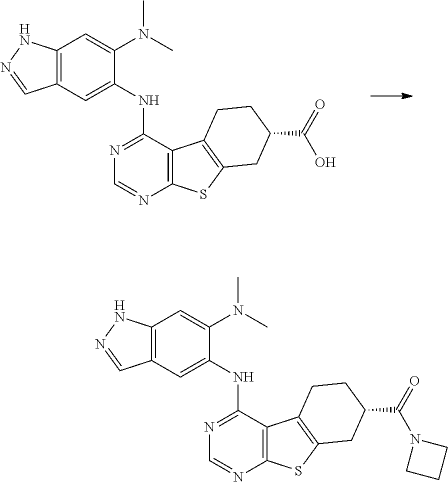 [SMILES: CN(C)c1cc2[nH]ncc2cc1Nc1ncnc2sc3c(c12)CC[C@H](C(=O)O)C3 ; CN(C)c1cc2[nH]ncc2cc1Nc1ncnc2sc3c(c12)CC[C@H](C(=O)N1CCC1)C3]

70 mg (171 μmol) (7S)-4-{[6-(dimethylamino)-1H-indazol-5-yl]amino}-5,6,7,8-tetrahydro[1]benzothieno[2,3-d]pyrimidine-7-carboxylic acid (prepared according to intermediate example 187a) were transformed in analogy to example 1 using azetidine to give after working up and purification 50.3 mg (62%) of the title compound.

¹H-NMR (DMSO-d6): δ=1.82 (1H), 2.15 (1H), 2.21 (2H), 2.72 (6H), 2.79 (1H), 2.90 (2H), 3.13 (1H), 3.26 (1H), 3.88 (2H), 4.25 (2H), 7.42 (1H), 8.03 (1H), 8.51 (1H), 8.99 (1H), 9.14 (1H), 12.90 (1H) ppm.

### Example 197

### (7S)—N-Ethyl-N-methoxy-4-[(6-methoxy-1H-indazol-5-yl)amino]-5,6,7,8-tetrahydro[1]benzothieno[2,3-d]pyrimidine-7-carboxamide

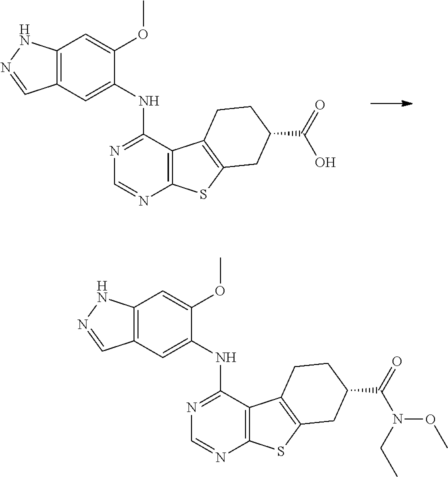 [SMILES: COc1cc2[nH]ncc2cc1Nc1ncnc2sc3c(c12)CC[C@H](C(=O)O)C3 ; CCN(OC)C(=O)[C@H]1CCc2c(sc3ncnc(Nc4cc5cn[nH]c5cc4OC)c23)C1]

70 mg (177 μmol) (7S)-4-[(6-methoxy-1H-indazol-5-yl)amino]-5,6,7,8-tetrahydro[1]benzothieno[2,3-d]pyrimidine-7-carboxylic acid (prepared according to intermediate example 20a) were transformed in analogy to example 1 using N-methoxyethanamine to give after working up and purification 55.3 mg (66%) of the title compound.

¹H-NMR (DMSO-d6): δ=1.10+1.20 (3H), 1.85 (1H), 2.17 (1H), 2.88-3.01 (2H), 3.12-3.34 (3H), 3.17+3.72 (3H), 3.53-4.02 (2H), 3.97 (3H), 7.09 (1H), 7.99 (1H), 8.21 (1H), 8.45+8.46 (1H), 8.74+8.77 (1H), 12.84 (1H) ppm.

### Example 198

### (7S)—N-Methoxy-4-[(6-methoxy-1H-indazol-5-yl)amino]-N-methyl-5,6,7,8-tetrahydro[1]benzothieno[2,3-d]pyrimidine-7-carboxamide

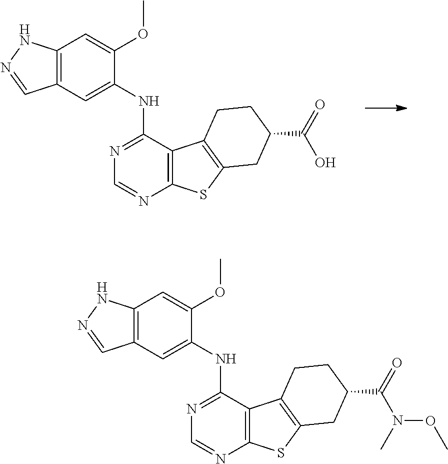 [SMILES: COc1cc2[nH]ncc2cc1Nc1ncnc2sc3c(c12)CC[C@H](C(=O)O)C3 ; COc1cc2[nH]ncc2cc1Nc1ncnc2sc3c(c12)CC[C@H](C(=O)N(C)OC)C3]

70 mg (177 μmol) (7S)-4-[(6-methoxy-1H-indazol-5-yl)amino]-5,6,7,8-tetrahydro[1]benzothieno[2,3-d]pyrimidine-7-carboxylic acid (prepared according to intermediate example 20a) were transformed in analogy to example 1 using N-methoxymethanamine to give after working up and purification 64.0 mg (78%) of the title compound.

¹H-NMR (DMSO-d6): δ=1.86 (1H), 2.19 (1H), 2.88-3.05 (2H), 3.13-3.27 (6H), 3.73 (3H), 3.97 (3H), 7.09 (1H), 7.99 (1H), 8.21 (1H), 8.45 (1H), 8.76 (1H), 12.84 (1H) ppm.

### Example 199

### (7S)-4-{[6-(Cyclopentyloxy)-1H-indazol-5-yl]amino}-N,N-dimethyl-5,6,7,8-tetrahydro[1]benzothieno[2,3-d]pyrimidine-7-carboxamide

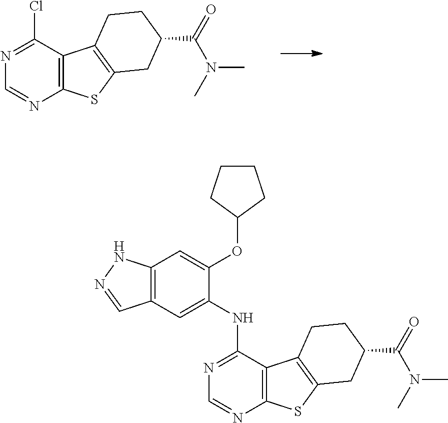 [SMILES: CN(C)C(=O)[C@H]1CCc2c(sc3ncnc(Cl)c23)C1 ; CN(C)C(=O)[C@H]1CCc2c(sc3ncnc(Nc4cc5cn[nH]c5cc4OC4CCCC4)c23)C1]

75 mg (254 μmol) (7S)-4-chloro-N,N-dimethyl-5,6,7,8-tetrahydro[1]benzothieno[2,3-d]pyrimidine-7-carboxamide (prepared according to intermediate example 94a) were transformed in analogy to intermediate example 1b using 6-(cyclopentyloxy)-1H-indazol-5-amine (prepared according to intermediate example 199a) to give after working up and purification 90.9 mg (75%) of the title compound.

¹H-NMR (DMSO-d6): δ=1.60-2.11 (10H), 2.87 (3H), 2.92 (2H), 3.09 (3H), 3.14-3.27 (3H), 5.06 (1H), 7.04 (1H), 7.98 (1H), 8.26 (1H), 8.51 (1H), 9.05 (1H), 12.77 (1H) ppm.

### Example 199a

### 6-(Cyclopentyloxy)-1H-indazol-5-amine

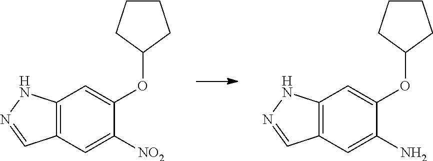 [SMILES: O=[N+]([O-])c1cc2cn[nH]c2cc1OC1CCCC1 ; Nc1cc2cn[nH]c2cc1OC1CCCC1]

765 mg (3.09 mmol) 6-(cyclopentyloxy)-5-nitro-1H-indazole (prepared according to intermediate example 199b) were transformed in analogy to intermediate example 94b to give after working up and purification 303 mg (45%) of the title compound.

### Example 199b

### 6-(Cyclopentyloxy)-5-nitro-1H-indazole

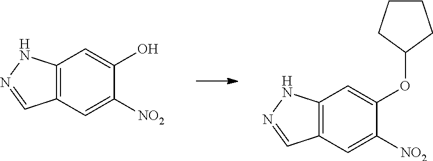 [SMILES: O=[N+]([O-])c1cc2cn[nH]c2cc1O ; O=[N+]([O-])c1cc2cn[nH]c2cc1OC1CCCC1]

675 mg (3.77 mmol) 5-nitro-1H-indazol-6-ol (prepared according to intermediate example 94d) were transformed in analogy to intermediate example 94c using cyclopentanol to give after working up and purification 768 mg (78%) of the title compound.

### Example 200

### (7S)—N,N-Dimethyl-4-{[6-(tetrahydro-2H-pyran-4-yloxy)-1H-indazol-5-yl]amino}-5,6,7,8-tetrahydro[1]benzothieno[2,3-d]pyrimidine-7-carboxamide

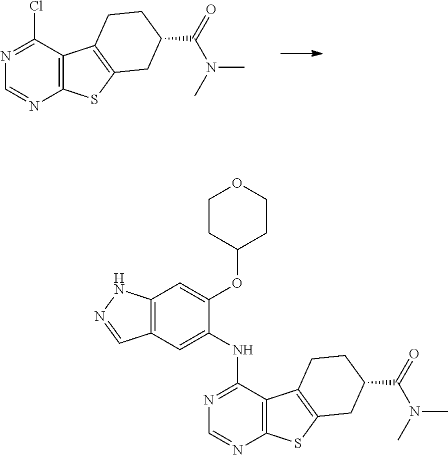 [SMILES: CN(C)C(=O)[C@H]1CCc2c(sc3ncnc(Cl)c23)C1 ; CN(C)C(=O)[C@H]1CCc2c(sc3ncnc(Nc4cc5cn[nH]c5cc4OC4CCOCC4)c23)C1]

75 mg (254 μmol) (7S)-4-chloro-N,N-dimethyl-5,6,7,8-tetrahydro[1]benzothieno[2,3-d]pyrimidine-7-carboxamide (prepared according to intermediate example 94a) were transformed in analogy to intermediate example 1b using 6-(tetrahydro-2H-pyran-4-yloxy)-1H-indazol-5-amine (prepared according to intermediate example 200a) to give after working up and purification 96.6 mg (77%) of the title compound.

¹H-NMR (DMSO-d6): δ=1.68 (2H), 1.87 (1H), 2.03-2.20 (3H), 2.87 (3H), 2.92 (2H), 3.10 (3H), 3.14-3.29 (3H), 3.52 (2H), 3.85-3.97 (2H), 4.83 (1H), 7.21 (1H), 7.99 (1H), 8.29 (1H), 8.51 (1H), 9.02 (1H), 12.78 (1H) ppm.

### Example 200a

### 6-(Tetrahydro-2H-pyran-4-yloxy)-1H-indazol-5-amine

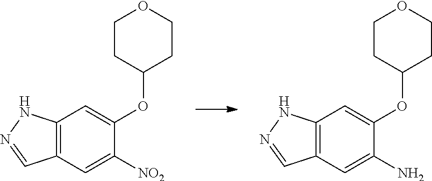 [SMILES: O=[N+]([O-])c1cc2cn[nH]c2cc1OC1CCOCC1 ; Nc1cc2cn[nH]c2cc1OC1CCOCC1]

533 mg (2.02 mmol) 5-nitro-6-(tetrahydro-2H-pyran-4-yloxy)-1H-indazole (prepared according to intermediate example 200b) were transformed in analogy to intermediate example 94b to give after working up and purification 311 mg (66%) of the title compound.

### Example 200b

### 5-Nitro-6-(Tetrahydro-2H-pyran-4-yloxy)-1H-indazole

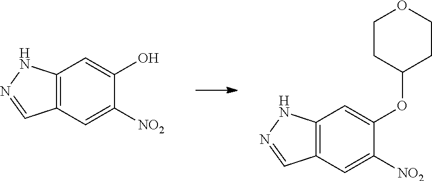 [SMILES: O=[N+]([O-])c1cc2cn[nH]c2cc1O ; O=[N+]([O-])c1cc2cn[nH]c2cc1OC1CCOCC1]

675 mg (3.77 mmol) 5-nitro-1H-indazol-6-ol (prepared according to intermediate example 94d) were transformed in analogy to intermediate example 94c using tetrahydro-2H-pyran-4-ol to give after working up and purification 592 mg (54%) of the title compound.

### Example 201

### (7S)—N,N-Dimethyl-4-{[6-(tetrahydro-2H-pyran-4-ylmethoxy)-1H-indazol-5-yl]amino}-5,6,7,8-tetrahydro[1]benzothieno[2,3-d]pyrimidine-7-carboxamide

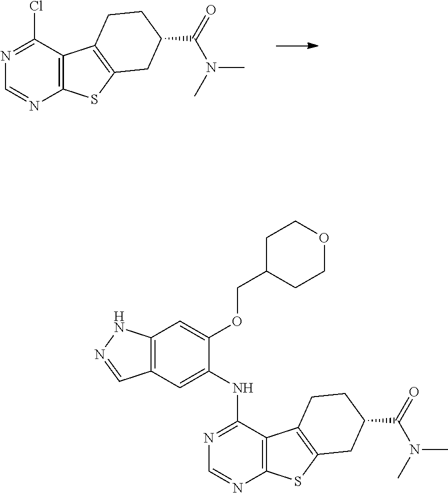 [SMILES: CN(C)C(=O)[C@H]1CCc2c(sc3ncnc(Cl)c23)C1 ; CN(C)C(=O)[C@H]1CCc2c(sc3ncnc(Nc4cc5cn[nH]c5cc4OCC4CCOCC4)c23)C1]

75 mg (254 μmol) (7S)-4-chloro-N,N-dimethyl-5,6,7,8-tetrahydro[1]benzothieno[2,3-d]pyrimidine-7-carboxamide (prepared according to intermediate example 94a) were transformed in analogy to intermediate example 1b using 6-(tetrahydro-2H-pyran-4-ylmethoxy)-1H-indazol-5-amine (prepared according to intermediate example 201a) to give after working up and purification 85.8 mg (67%) of the title compound.

¹H-NMR (DMSO-d6): δ=1.39 (2H), 1.71 (2H), 1.82 (1H), 2.07 (2H), 2.88 (3H), 2.92 (2H), 3.10 (3H), 3.15-3.39 (5H), 3.90 (2H), 4.02 (2H), 7.07 (1H), 7.99 (1H), 8.15 (1H), 8.49 (1H), 8.97 (1H), 12.83 (1H) ppm.

### Example 201a

### 6-(Tetrahydro-2H-pyran-4-ylmethoxy)-1H-indazol-5-amine

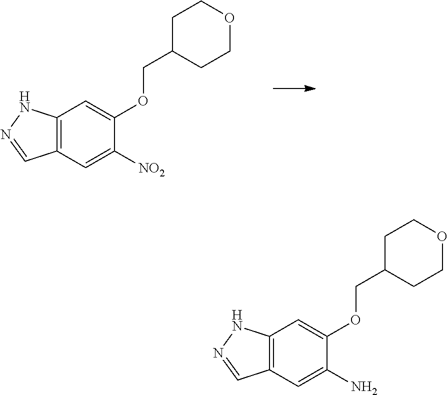 [SMILES: O=[N+]([O-])c1cc2cn[nH]c2cc1OCC1CCOCC1 ; Nc1cc2cn[nH]c2cc1OCC1CCOCC1]

387 mg (1.40 mmol) 5-nitro-6-(tetrahydro-2H-pyran-4-ylmethoxy)-1H-indazole (prepared according to intermediate example 200b) were transformed in analogy to intermediate example 201b to give after working up and purification 159 mg (41%) of the title compound.

### Example 201b

### 5-Nitro-6-(tetrahydro-2H-pyran-4-ylmethoxy)-1H-indazole

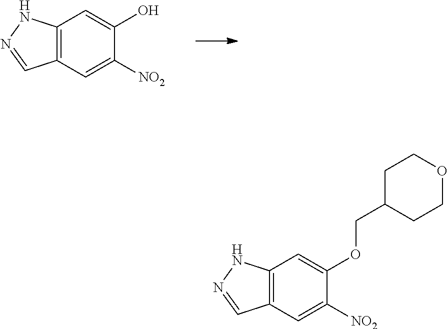 [SMILES: O=[N+]([O-])c1cc2cn[nH]c2cc1O ; O=[N+]([O-])c1cc2cn[nH]c2cc1OCC1CCOCC1]

675 mg (3.77 mmol) 5-nitro-1H-indazol-6-ol (prepared according to intermediate example 94d) were transformed in analogy to intermediate example 94c using tetrahydro-2H-pyran-4-ylmethanol to give after working up and purification 434 mg (37%) of the title compound.

### Example 202

### (7S)-4-{[6-(Cyclohexyloxy)-1H-indazol-5-yl]amino}-N,N-dimethyl-5,6,7,8-tetrahydro[1]benzothieno[2,3-d]pyrimidine-7-carboxamide

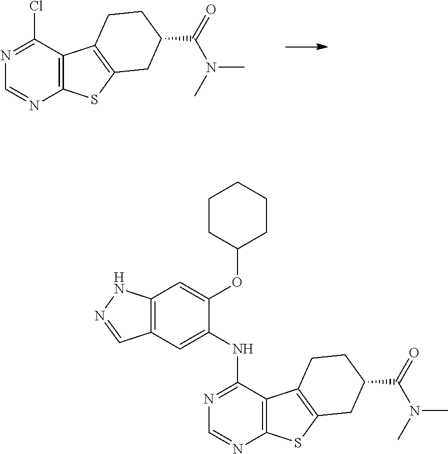 [SMILES: CN(C)C(=O)[C@H]1CCc2c(sc3ncnc(Cl)c23)C1 ; CN(C)C(=O)[C@H]1CCc2c(sc3ncnc(Nc4cc5cn[nH]c5cc4OC4CCCCC4)c23)C1]

75 mg (254 μmol) (7S)-4-chloro-N,N-dimethyl-5,6,7,8-tetrahydro[1]benzothieno[2,3-d]pyrimidine-7-carboxamide (prepared according to intermediate example 94a) were transformed in analogy to intermediate example 1b using 6-(cyclohexyloxy)-1H-indazol-5-amine (prepared according to intermediate example 202a) to give after working up and purification 50.1 mg (40%) of the title compound.

¹H-NMR (DMSO-d6): δ=1.23 (1H), 1.36-1.53 (4H), 1.62 (1H), 1.71-1.93 (3H), 2.02-2.22 (3H), 2.87 (3H), 2.92 (2H), 3.10 (3H), 3.14-3.29 (3H), 4.57 (1H), 7.14 (1H), 7.98 (1H), 8.32 (1H), 8.51 (1H), 9.02 (1H), 12.73 (1H) ppm.

### Example 202a

### 6-(Cyclohexyloxy)-1H-indazol-5-amine

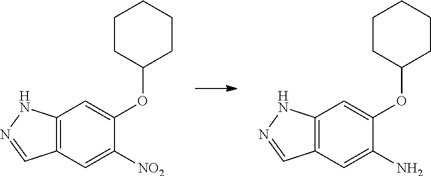 [SMILES: O=[N+]([O-])c1cc2cn[nH]c2cc1OC1CCCCC1 ; Nc1cc2cn[nH]c2cc1OC1CCCCC1]

311 mg (1.19 mmol) 6-(cyclohexyloxy)-5-nitro-1H-indazole (prepared according to intermediate example 202b) were transformed in analogy to intermediate example 94b to give after working up and purification 137 mg (50%) of the title compound.

### Example 202b

6-(Cyclohexyloxy)-5-nitro-1H-indazole

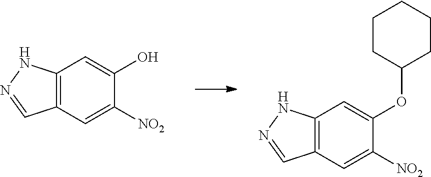 [SMILES: O=[N+]([O-])c1cc2cn[nH]c2cc1O ; O=[N+]([O-])c1cc2cn[nH]c2cc1OC1CCCCC1]

675 mg (3.77 mmol) 5-nitro-1H-indazol-6-ol (prepared according to intermediate example 94d) were transformed in analogy to intermediate example 94c using cyclohexanol to give after working up and purification 349 mg (32%) of the title compound.

### Example 203

### (7S)-4-({6-[3-(Dimethylamino)propoxy]-1H-indazol-5-yl}amino)-N,N-dimethyl-5,6,7,8-tetrahydro[1]benzothieno[2,3-d]pyrimidine-7-carboxamide

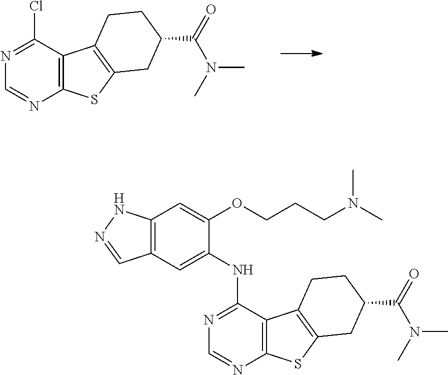 [SMILES: CN(C)C(=O)[C@H]1CCc2c(sc3ncnc(Cl)c23)C1 ; CN(C)CCCOc1cc2[nH]ncc2cc1Nc1ncnc2sc3c(c12)CC[C@H](C(=O)N(C)C)C3]

75 mg (254 μmol) (7S)-4-chloro-N,N-dimethyl-5,6,7,8-tetrahydro[1]benzothieno[2,3-d]pyrimidine-7-carboxamide (prepared according to intermediate example 94a) were transformed in analogy to intermediate example 1b using 6-[3-(dimethylamino)propoxy]-1H-indazol-5-amine (prepared according to intermediate example 203a) to give after working up and purification 43.1 mg (33%) of the title compound.

¹H-NMR (DMSO-d6): δ=1.85 (1H), 2.10-2.21 (3H), 2.56 (6H), 2.85-3.01 (4H), 2.90 (3H), 3.12 (3H), 3.17-3.30 (3H), 4.24 (2H), 7.11 (1H), 8.02 (1H), 8.23 (1H), 8.50 (1H), 8.86 (1H), 12.90 (1H) ppm.

### Example 203a

### 6-[3-(Dimethylamino)propoxy]-1H-indazol-5-amine

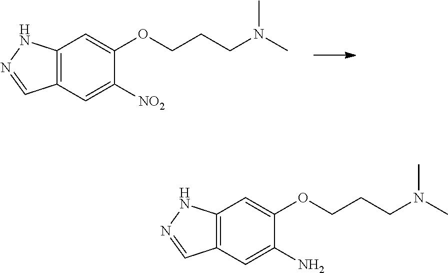 [SMILES: CN(C)CCCOc1cc2[nH]ncc2cc1[N+](=O)[O-] ; CN(C)CCCOc1cc2[nH]ncc2cc1N]

495 mg (1.87 mmol) N,N-dimethyl-3-[(5-nitro-1H-indazol-6-yl)oxy]propan-1-amine (prepared according to intermediate example 203b) were transformed in analogy to intermediate example 94b to give after working up and purification 326 mg (74%) of the title compound.

### Example 203b

### N,N-Dimethyl-3-[(5-nitro-1H-indazol-6-yl)oxy]propan-1-amine

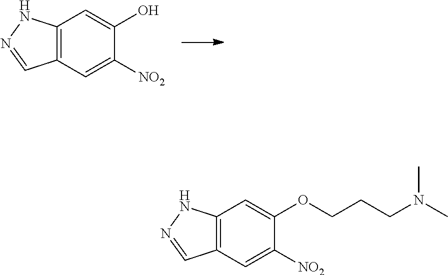 [SMILES: O=[N+]([O-])c1cc2cn[nH]c2cc1O ; CN(C)CCCOc1cc2[nH]ncc2cc1[N+](=O)[O-]]

675 mg (3.77 mmol) 5-nitro-1H-indazol-6-ol (prepared according to intermediate example 94d) were transformed in analogy to intermediate example 94c using 3-(dimethylamino)propan-1-ol to give after working up and purification 550 mg (55%) of the title compound.

### Example 204

### (7S)-4-[(6-Methoxy-1H-indazol-5-yl)amino]-N-(2-methoxy-2-methylpropyl)-N-methyl-5,6,7,8-tetrahydro[1]benzothieno[2,3-d]pyrimidine-7-carboxamide

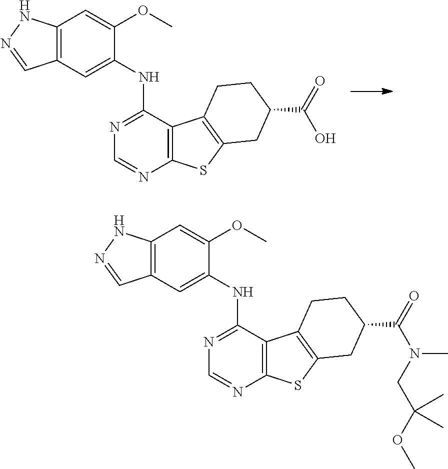 [SMILES: COc1cc2[nH]ncc2cc1Nc1ncnc2sc3c(c12)CC[C@H](C(=O)O)C3 ; COc1cc2[nH]ncc2cc1Nc1ncnc2sc3c(c12)CC[C@H](C(=O)N(C)CC(C)(C)OC)C3]

100 mg (253 μmol) (7S)-4-[(6-methoxy-1H-indazol-5-yl)amino]-5,6,7,8-tetrahydro[1]benzothieno[2,3-d]pyrimidine-7-carboxylic acid (prepared according to intermediate example 20a) were transformed in analogy to example 1 using 2-methoxy-N,2-dimethylpropan-1-amine (CAS-No: 17860-82-1) to give after working up and purification 65.0 mg (49%) of the title compound.

¹H-NMR (DMSO-d6): δ=1.04-1.15 (6H), 1.85 (1H), 2.07+2.17 (1H), 2.81-3.54 (13H), 3.97+3.99 (3H), 7.09 (1H), 7.99 (1H), 8.22 (1H), 8.45+8.46 (1H), 8.76+8.80 (1H), 12.84 (1H) ppm.

### Example 205

### [(3RS)-3-fluoropiperidin-1-yl]{(7S)-4-[(6-methoxy-1H-indazol-5-yl)amino]-5,6,7,8-tetrahydro[1]benzothieno[2,3-d]pyrimidin-7-yl}methanone

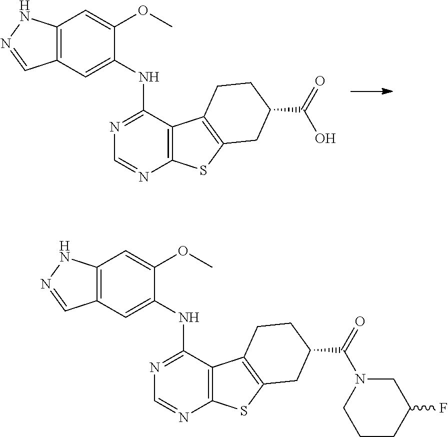 [SMILES: COc1cc2[nH]ncc2cc1Nc1ncnc2sc3c(c12)CC[C@H](C(=O)O)C3 ; COc1cc2[nH]ncc2cc1Nc1ncnc2sc3c(c12)CC[C@H](C(=O)N1CCCC(F)C1)C3]

A mixture comprising 60 mg (152 μmol) (7S)-4-[(6-methoxy-1H-indazol-5-yl)amino]-5,6,7,8-tetrahydro[1]benzothieno[2,3-d]pyrimidine-7-carboxylic acid (prepared according to intermediate example 20a), 2.2 mL dimethyl sulfoxide, 63.5 mg (3RS)-3-fluoropiperidine hydrochloride (CAS-No: 116574-75-5), 159 μL N-ethyl-N-isopropylpropan-2-amine and 722 μL (1H-benzotriazol-1-yloxy)(tripyrrolidin-1-yl)phosphonium hexafluorophosphate was heated at 90° C. overnight. The crude mixture was purified by chromatography to give 73.2 mg (90%) of the title compound.

¹H-NMR (DMSO-d6): δ=1.55 (1H), 1.76-1.97 (3H), 2.16 (1H), 2.84-3.08 (3H), 3.16-3.81 (6H), 3.95-4.11 (4H), 4.78 (1H), 7.10 (1H), 8.01 (1H), 8.23 (1H), 8.47 (1H), 8.78 (1H), 12.87 (1H) ppm.

### Example 206

### 2,5-Dihydro-1H-pyrrol-1-yl{(7S)-4-[(6-methoxy-1H-indazol-5-yl)amino]-5,6,7,8-tetrahydro[1]benzothieno[2,3-d]pyrimidin-7-yl}methanone

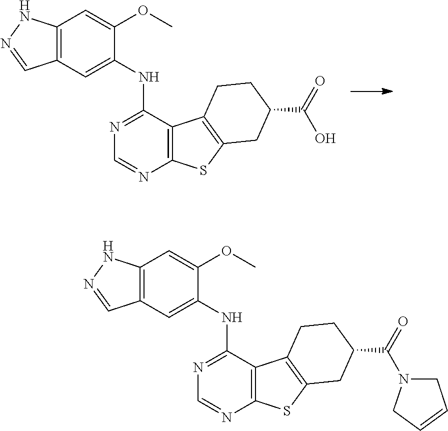 [SMILES: COc1cc2[nH]ncc2cc1Nc1ncnc2sc3c(c12)CC[C@H](C(=O)O)C3 ; COc1cc2[nH]ncc2cc1Nc1ncnc2sc3c(c12)CC[C@H](C(=O)N1CC=CC1)C3]

100 mg (253 μmol) (7S)-4-[(6-methoxy-1H-indazol-5-yl)amino]-5,6,7,8-tetrahydro[1]benzothieno[2,3-d]pyrimidine-7-carboxylic acid (prepared according to intermediate example 20a) were transformed in analogy to example 205 using 2,5-dihydro-1H-pyrrole to give after working up and purification 87.0 mg (73%) of the title compound.

¹H-NMR (DMSO-d6): δ=1.86 (1H), 2.24 (1H), 2.95-3.05 (3H), 3.14-3.39 (2H), 4.00 (3H), 4.14 (2H), 4.44 (2H), 5.95 (2H), 7.11 (1H), 8.01 (1H), 8.26 (1H), 8.48 (1H), 8.80 (1H), 12.87 (1H) ppm.

### Example 207

### {(7S)-4-[(6-methoxy-1H-indazol-5-yl)amino]-5,6,7,8-tetrahydro[1]benzothieno[2,3-d]pyrimidin-7-yl}(thiomorpholin-4-yl)methanone

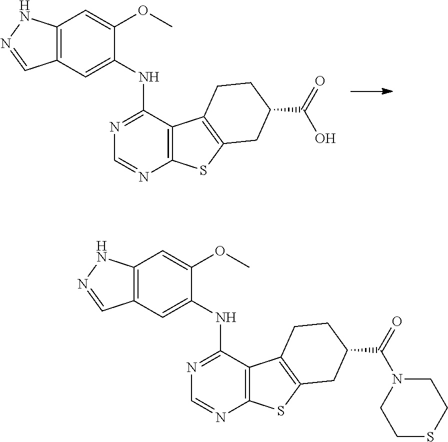 [SMILES: COc1cc2[nH]ncc2cc1Nc1ncnc2sc3c(c12)CC[C@H](C(=O)O)C3 ; COc1cc2[nH]ncc2cc1Nc1ncnc2sc3c(c12)CC[C@H](C(=O)N1CCSCC1)C3]

100 mg (253 μmol) (7S)-4-[(6-methoxy-1H-indazol-5-yl)amino]-5,6,7,8-tetrahydro[1]benzothieno[2,3-d]pyrimidine-7-carboxylic acid (prepared according to intermediate example 20a) were transformed in analogy to example 205 using thiomorpholine (CAS-No: 123-90-0) to give after working up and purification 80.0 mg (63%) of the title compound.

¹H-NMR (DMSO-d6): δ=1.87 (1H), 2.15 (1H), 2.59 (4H), 2.90-3.04 (2H), 3.17-3.30 (3H), 3.68-3.94 (4H), 4.00 (3H), 7.11 (1H), 8.01 (1H), 8.24 (1H), 8.48 (1H), 8.78 (1H), 12.87 (1H) ppm.

### Example 208

### (3,3-Difluoropyrrolidin-1-yl){(7S)-4-[(6-methoxy-1H-indazol-5-yl)amino]-5,6,7,8-tetrahydro[1]benzothieno[2,3-d]pyrimidin-7-yl}methanone

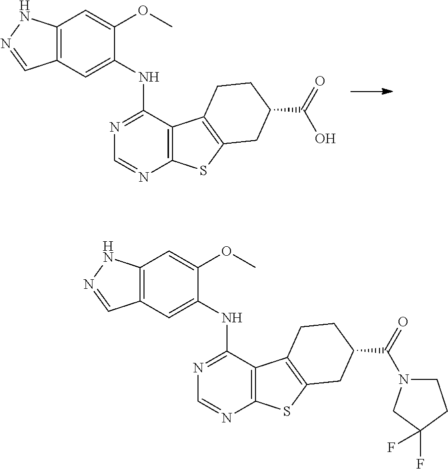 [SMILES: COc1cc2[nH]ncc2cc1Nc1ncnc2sc3c(c12)CC[C@H](C(=O)O)C3 ; COc1cc2[nH]ncc2cc1Nc1ncnc2sc3c(c12)CC[C@H](C(=O)N1CCC(F)(F)C1)C3]

100 mg (253 μmol) (7S)-4-[(6-methoxy-1H-indazol-5-yl)amino]-5,6,7,8-tetrahydro[1]benzothieno[2,3-d]pyrimidine-7-carboxylic acid (prepared according to intermediate example 20a) were transformed in analogy to example 1 using 3,3-difluoropyrrolidine (CAS-No: 316131-01-8) to give after working up and purification 41.4 mg (32%) of the title compound.

¹H-NMR (DMSO-d6): δ=1.85 (1H), 2.21 (1H), 2.36-2.60 (2H), 2.91-3.42 (4H), 3.50-3.63 (2H), 3.77 (1H), 3.88 (1H), 4.00 (3H), 4.13 (1H), 7.11 (1H), 8.01 (1H), 8.25 (1H), 8.48 (1H), 8.79 (1H), 12.87 (1H) ppm.

### Example 209

### 3-Azabicyclo[3.1.0]hex-3-yl{(7S)-4-[(6-methoxy-1H-indazol-5-yl)amino]-5,6,7,8-tetrahydro[1]benzothieno[2,3-d]pyrimidin-7-yl}methanone

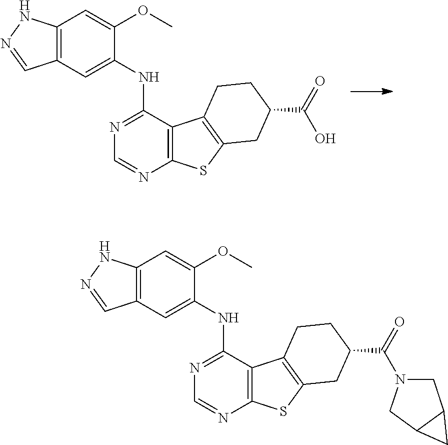 [SMILES: COc1cc2[nH]ncc2cc1Nc1ncnc2sc3c(c12)CC[C@H](C(=O)O)C3 ; COc1cc2[nH]ncc2cc1Nc1ncnc2sc3c(c12)CC[C@H](C(=O)N1CC2CC2C1)C3]

100 mg (253 μmol) (7S)-4-[(6-methoxy-1H-indazol-5-yl)amino]-5,6,7,8-tetrahydro[1]benzothieno[2,3-d]pyrimidine-7-carboxylic acid (prepared according to intermediate example 20a) were transformed in analogy to example 205 using 3-azabicyclo[3.1.0]hexane (CAS-No: 285-59-6) to give after working up and purification 28.3 mg (23%) of the title compound.

¹H-NMR (DMSO-d6): δ=0.72 (1H), 1.55 (1H), 1.63 (1H), 1.81 (1H), 2.13 (1H), 2.92 (3H), 3.10-3.36 (3H), 3.31 (1H), 3.60-3.69 (2H), 3.75 (1H), 3.99 (3H), 7.09 (1H), 7.99 (1H), 8.21 (1H), 8.46 (1H), 8.78 (1H), 12.84 (1H) ppm.

### Example 210

### [(2S,6S)-2,6-Dimethylmorpholin-4-yl]{(7S)-4-[(6-methoxy-1H-indazol-5-yl)amino]-5,6,7,8-tetrahydro[1]benzothieno[2,3-d]pyrimidin-7-yl}methanone

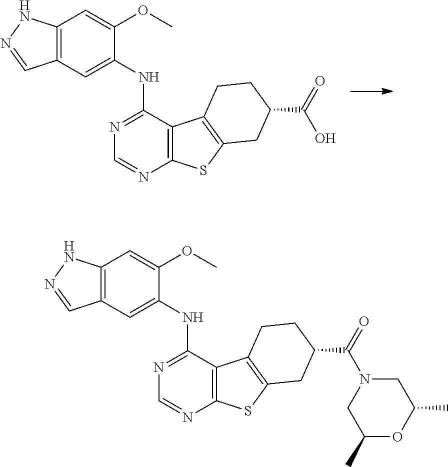 [SMILES: COc1cc2[nH]ncc2cc1Nc1ncnc2sc3c(c12)CC[C@H](C(=O)O)C3 ; COc1cc2[nH]ncc2cc1Nc1ncnc2sc3c(c12)CC[C@H](C(=O)N1C[C@H](C)O[C@@H](C)C1)C3]

50 mg (126 μmol) (7S)-4-[(6-methoxy-1H-indazol-5-yl)amino]-5,6,7,8-tetrahydro[1]benzothieno[2,3-d]pyrimidine-7-carboxylic acid (prepared according to intermediate example 20a) were transformed in analogy to example 1 using (2S,6S)-2,6-dimethylmorpholine (CAS-No: 276252-73-4) to give after working up and purification 46.2 mg (70%) of the title compound.

¹H-NMR (DMSO-d6): δ=1.11 (3H), 1.14 (3H), 1.91 (1H), 2.13 (1H), 2.91-3.02 (2H), 3.16-3.42 (5H), 3.48 (1H), 3.75 (1H), 3.95 (2H), 3.99 (3H), 7.10 (1H), 8.01 (1H), 8.24 (1H), 8.47 (1H), 8.77 (1H), 12.87 (1H) ppm.

### Example 211

### [(2R,6R)-2,6-Dimethylmorpholin-4-yl]{(7S)-4-[(6-methoxy-1H-indazol-5-yl)amino]-5,6,7,8-tetrahydro[1]benzothieno[2,3-d]pyrimidin-7-yl}methanone

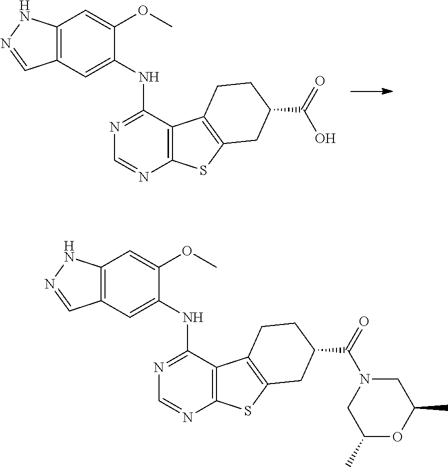 [SMILES: COc1cc2[nH]ncc2cc1Nc1ncnc2sc3c(c12)CC[C@H](C(=O)O)C3 ; COc1cc2[nH]ncc2cc1Nc1ncnc2sc3c(c12)CC[C@H](C(=O)N1C[C@@H](C)O[C@H](C)C1)C3]

50 mg (126 μmol) (7S)-4-[(6-methoxy-1H-indazol-5-yl)amino]-5,6,7,8-tetrahydro[1]benzothieno[2,3-d]pyrimidine-7-carboxylic acid (prepared according to intermediate example 20a) were transformed in analogy to example 1 using (2R,6R)-2,6-dimethylmorpholine (CAS-No: 171753-74-5) to give after working up and purification 30.1 mg (46%) of the title compound.

¹H-NMR (DMSO-d6): δ=1.11 (3H), 1.14 (3H), 1.88 (1H), 2.15 (1H), 2.90 (1H), 3.02 (1H), 3.18-3.40 (5H), 3.55 (1H), 3.73 (1H), 3.95 (2H), 4.00 (3H), 7.11 (1H), 8.01 (1H), 8.23 (1H), 8.47 (1H), 8.78 (1H), 12.87 (1H) ppm.

### Example 212

### {(7S)-4-[(6-Methoxy-1H-indazol-5-yl)amino]-5,6,7,8-tetrahydro[1]benzothieno[2,3-d]pyrimidin-7-yl}(1,2-oxazinan-2-yl)methanone

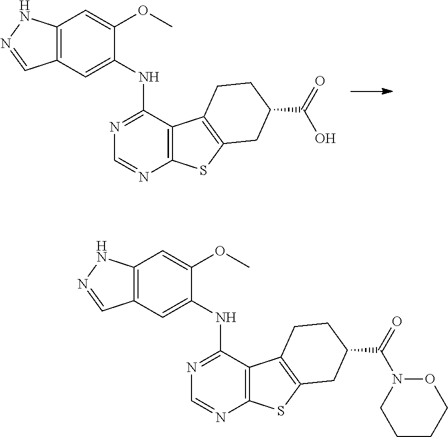 [SMILES: COc1cc2[nH]ncc2cc1Nc1ncnc2sc3c(c12)CC[C@H](C(=O)O)C3 ; COc1cc2[nH]ncc2cc1Nc1ncnc2sc3c(c12)CC[C@H](C(=O)N1CCCCO1)C3]

70 mg (177 μmol) (7S)-4-[(6-methoxy-1H-indazol-5-yl)amino]-5,6,7,8-tetrahydro[1]benzothieno[2,3-d]pyrimidine-7-carboxylic acid (prepared according to intermediate example 20a) were transformed in analogy to example 1 using 1,2-oxazinane (CAS-No: 36652-42-3) to give after working up and purification 18.4 mg (21%) of the title compound.

¹H-NMR (DMSO-d6): δ=1.66 (2H), 1.77 (2H), 1.87 (1H), 2.18 (1H), 2.88-3.03 (2H), 3.12-3.28 (3H), 3.65 (1H), 3.76 (1H), 3.97 (3H), 4.02 (2H), 7.09 (1H), 7.99 (1H), 8.21 (1H), 8.45 (1H), 8.76 (1H), 12.83 (1H) ppm.

### Example 213

### {(7S)-4-[(6-Methoxy-1H-indazol-5-yl)amino]-5,6,7,8-tetrahydro[1]benzothieno[2,3-d]pyrimidin-7-yl}(1,2-oxazolidin-2-yl)methanone

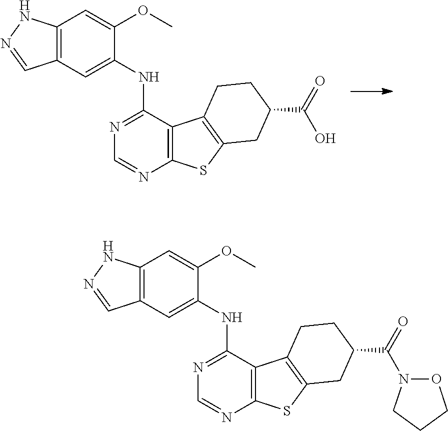 [SMILES: COc1cc2[nH]ncc2cc1Nc1ncnc2sc3c(c12)CC[C@H](C(=O)O)C3 ; COc1cc2[nH]ncc2cc1Nc1ncnc2sc3c(c12)CC[C@H](C(=O)N1CCCO1)C3]

70 mg (177 μmol) (7S)-4-[(6-methoxy-1H-indazol-5-yl)amino]-5,6,7,8-tetrahydro[1]benzothieno[2,3-d]pyrimidine-7-carboxylic acid (prepared according to intermediate example 20a) were transformed in analogy to example 1 using 1,2-oxazolidine (CAS-No: 504-72-3) to give after working up and purification 69.0 mg (82%) of the title compound.

¹H-NMR (DMSO-d6): δ=1.89 (1H), 2.16-2.33 (3H), 2.87-3.04 (2H), 3.09-3.27 (3H), 3.55-3.72 (2H), 3.92-4.07 (2H), 3.97 (3H), 7.08 (1H), 7.99 (1H), 8.21 (1H), 8.45 (1H), 8.76 (1H), 12.86 (1H) ppm.

### Example 214

### (7S)—N-Ethoxy-4-[(6-methoxy-1H-indazol-5-yl)amino]-N-methyl-5,6,7,8-tetrahydro[1]benzothieno[2,3-d]pyrimidine-7-carboxamide

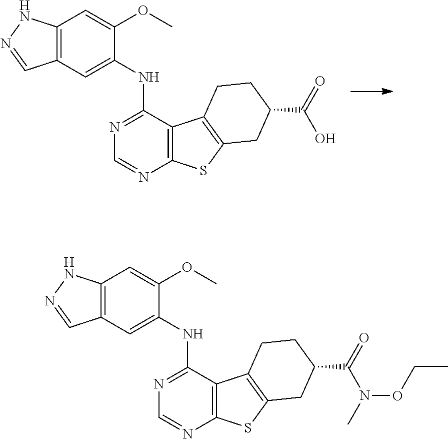 [SMILES: COc1cc2[nH]ncc2cc1Nc1ncnc2sc3c(c12)CC[C@H](C(=O)O)C3 ; CCON(C)C(=O)[C@H]1CCc2c(sc3ncnc(Nc4cc5cn[nH]c5cc4OC)c23)C1]

70 mg (177 μmol) (7S)-4-[(6-methoxy-1H-indazol-5-yl)amino]-5,6,7,8-tetrahydro[1]benzothieno[2,3-d]pyrimidine-7-carboxylic acid (prepared according to intermediate example 20a) were transformed in analogy to example 1 using N-ethoxymethanamine (UkrOrgSynthesis Ltd., http://www.ukrorgsynth.com) to give after working up and purification 59.5 mg (71%) of the title compound.

¹H-NMR (DMSO-d6): δ=1.20 (3H), 1.85 (1H), 2.19 (1H), 2.95 (2H), 3.11-3.41 (3H), 3.17 (3H), 3.89-4.07 (2H), 3.96 (3H), 7.08 (1H), 7.99 (1H), 8.21 (1H), 8.45 (1H), 8.74 (1H), 12.86 (1H) ppm.

### Example 215

### (7S)—N-(3-Hydroxy-3-methylbutyl)-4-[(6-methoxy-1H-indazol-5-yl)amino]-N-methyl-5,6,7,8-tetrahydro[1]benzothieno[2,3-d]pyrimidine-7-carboxamide

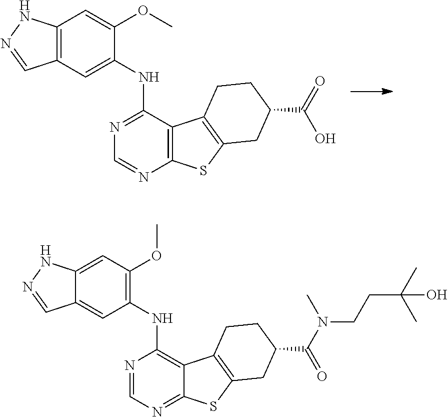 [SMILES: COc1cc2[nH]ncc2cc1Nc1ncnc2sc3c(c12)CC[C@H](C(=O)O)C3 ; COc1cc2[nH]ncc2cc1Nc1ncnc2sc3c(c12)CC[C@H](C(=O)N(C)CCC(C)(C)O)C3]

100 mg (253 μmol) (7S)-4-[(6-methoxy-1H-indazol-5-yl)amino]-5,6,7,8-tetrahydro[1]benzothieno[2,3-d]pyrimidine-7-carboxylic acid (prepared according to intermediate example 20a) were transformed in analogy to example 1 using 2-methyl-4-(methylamino)butan-2-ol (CAS-No: 866223-53-2) to give after working up and purification 62.4 mg (46%) of the title compound.

¹H-NMR (DMSO-d6): δ=1.13 (6H), 1.56 (1H), 1.68 (1H), 1.85 (1H), 2.15 (1H), 2.86+3.09 (3H), 2.88-3.04 (2H), 3.11-3.37 (4H), 3.47 (1H), 3.99+4.00 (3H), 4.28+4.41 (1H), 7.11 (1H), 8.01 (1H), 8.24 (1H), 8.47+8.48 (1H), 8.77+8.80 (1H), 12.88 (1H) ppm.

### Example 216

### (7S)-4-[(6-Methoxy-1H-indazol-5-yl)amino]-N-methyl-N-(methylsulfonyl)-5,6,7,8-tetrahydro[1]benzothieno[2,3-d]pyrimidine-7-carboxamide

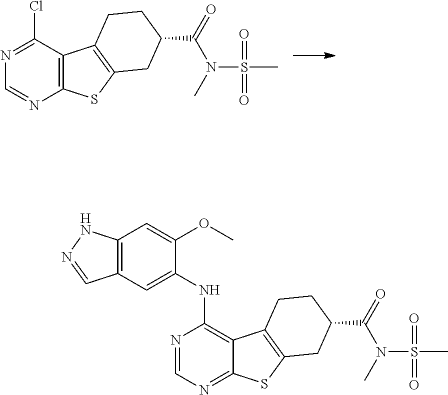 [SMILES: CN(C(=O)[C@H]1CCc2c(sc3ncnc(Cl)c23)C1)S(C)(=O)=O ; COc1cc2[nH]ncc2cc1Nc1ncnc2sc3c(c12)CC[C@H](C(=O)N(C)S(C)(=O)=O)C3]

19.1 mg (53 μmol) (7S)-4-chloro-N-methyl-N-(methylsulfonyl)-5,6,7,8-tetrahydro[1]benzothieno[2,3-d]pyrimidine-7-carboxamide (prepared according to intermediate example 216a) were transformed in analogy to intermediate example 1b using 6-methoxy-1H-indazol-5-amine to give after working up and purification 14.8 mg (57%) of the title compound.

¹H-NMR (DMSO-d6): δ=1.91 (1H), 2.32 (1H), 2.99 (1H), 3.10 (1H), 3.18-3.52 (2H), 3.31 (3H), 3.43 (3H), 3.48 (1H), 3.99 (3H), 7.10 (1H), 8.01 (1H), 8.23 (1H), 8.47 (1H), 8.75 (1H), 12.87 (1H) ppm.

### Example 216a

### (7S)-4-Chloro-N-methyl-N-(methylsulfonyl)-5,6,7,8-tetrahydro[1]benzothieno[2,3-d]pyrimidine-7-carboxamide

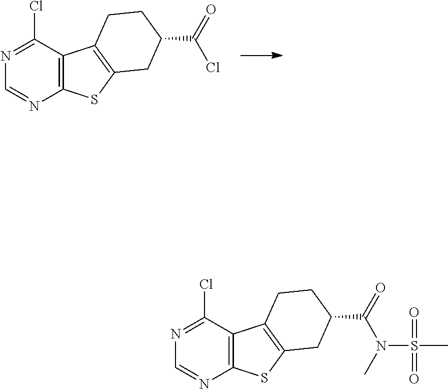 [SMILES: O=C(Cl)[C@H]1CCc2c(sc3ncnc(Cl)c23)C1 ; CN(C(=O)[C@H]1CCc2c(sc3ncnc(Cl)c23)C1)S(C)(=O)=O]

A mixture comprising 1.03 g (3.57 mmol) (7S)-4-chloro-5,6,7,8-tetrahydro [1]benzothieno[2,3-d]pyrimidine-7-carbonyl chloride (prepared according to intermediate example 216b), 36.6 mL N,N-dimethylformamide, 584 mg N-methylmethanesulfonamide and 1.86 mL N-ethyl-N-isopropylpropan-2-amine was stirred at 23° C. overnight. The crude mixture was purified by chromatography to give 186 mg (14%) of the title compound.

### Example 216b

### (7S)-4-Chloro-5,6,7,8-tetrahydro[1]benzothieno[2,3-d]pyrimidine-7-carbonyl chloride

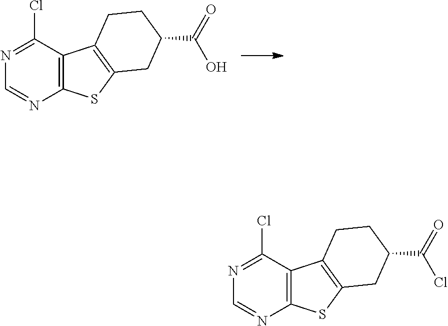 [SMILES: O=C(O)[C@H]1CCc2c(sc3ncnc(Cl)c23)C1 ; O=C(Cl)[C@H]1CCc2c(sc3ncnc(Cl)c23)C1]

881 mg (3.28 mmol) (7S)-4-[(6-methoxy-1H-indazol-5-yl)amino]-5,6,7,8-tetrahydro[1]benzothieno[2,3-d]pyrimidine-7-carboxylic acid (prepared according to intermediate example 20a) were transformed in analogy to intermediate example 1e to give after working up 1.02 g of the title compound as crude product that was used without further purification.

### Example 217

### (7S)—N-(2-Methoxyethyl)-4-[(6-methoxy-1H-indazol-5-yl)amino]-N-methyl-5,6,7,8-tetrahydro[1]benzothieno[2,3-d]pyrimidine-7-carboxamide

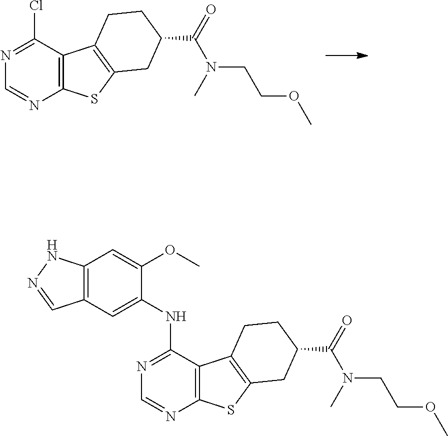 [SMILES: COCCN(C)C(=O)[C@H]1CCc2c(sc3ncnc(Cl)c23)C1 ; COCCN(C)C(=O)[C@H]1CCc2c(sc3ncnc(Nc4cc5cn[nH]c5cc4OC)c23)C1]

75 mg (221 μmol) (7S)-4-chloro-N-(2-methoxyethyl)-N-methyl-5,6,7,8-tetrahydro[1]benzothieno[2,3-d]pyrimidine-7-carboxamide (prepared according to intermediate example 217a) were transformed in analogy to intermediate example 1b using 6-methoxy-1H-indazol-5-amine to give after working up and purification 69.9 mg (64%) of the title compound.

¹H-NMR (DMSO-d6): δ=1.84 (1H), 2.14 (1H), 2.85-3.03 (2H), 2.89+3.15 (3H), 3.11-3.31 (3H), 3.27+3.29 (3H), 3.39-3.73 (4H), 3.98+3.99 (3H), 7.09 (1H), 8.01 (1H), 8.24 (1H), 8.47 (1H), 8.79 (1H), 12.89 (1H) ppm.

### Example 217a

### (7S)-4-Chloro-N-(2-methoxyethyl)-N-methyl-5,6,7,8-tetrahydro[1]benzothieno[2,3-d]pyrimidine-7-carboxamide

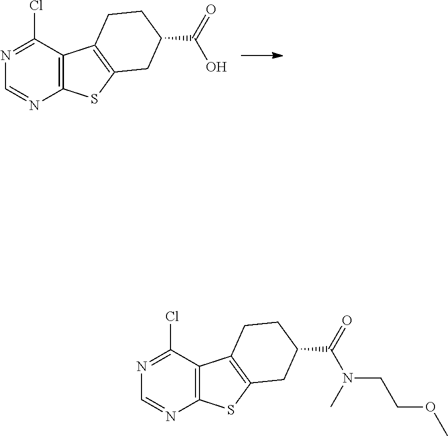 [SMILES: O=C(O)[C@H]1CCc2c(sc3ncnc(Cl)c23)C1 ; COCCN(C)C(=O)[C@H]1CCc2c(sc3ncnc(Cl)c23)C1]

3.50 g (13.0 mmol) (7S)-4-[(6-methoxy-1H-indazol-5-yl)amino]-5,6,7,8-tetrahydro[1]benzothieno[2,3-d]pyrimidine-7-carboxylic acid (prepared according to intermediate example 20a) were transformed in analogy to example 1 using 2-methoxy-N-methylethanamine to give after working up and purification 3.62 g (82%) of the title compound.

### Example 218

### [3-(Dimethylamino)azetidin-1-yl]{(7S)-4-[(6-ethoxy-1H-indazol-5-yl)amino]-5,6,7,8-tetrahydro[1]benzothieno[2,3-d]pyrimidin-7-yl}methanone

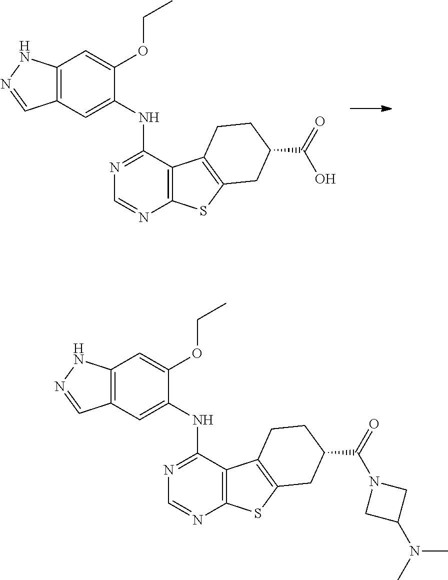 [SMILES: CCOc1cc2[nH]ncc2cc1Nc1ncnc2sc3c(c12)CC[C@H](C(=O)O)C3 ; CCOc1cc2[nH]ncc2cc1Nc1ncnc2sc3c(c12)CC[C@H](C(=O)N1CC(N(C)C)C1)C3]

50 mg (122 μmol) (7S)-4-[(6-ethoxy-1H-indazol-5-yl)amino]-5,6,7,8-tetrahydro[1]benzothieno[2,3-d]pyrimidine-7-carboxylic acid (prepared according to intermediate example 65a) were transformed in analogy to example 1 using N,N-dimethylazetidin-3-amine (CAS-No: 138022-85-2) to give after working up and purification 7.5 mg (12%) of the title compound.

¹H-NMR (DMSO-d6): δ=1.50 (3H), 1.86 (1H), 2.07 (1H), 2.10 (6H), 2.77-3.42 (6H), 3.67 (1H), 3.90 (1H), 3.96-4.14 (1H), 4.20-4.32 (3H), 7.08 (1H), 8.01 (1H), 8.38 (1H), 8.54 (1H), 9.03 (1H), 12.84 (1H) ppm.

### Example 219

### [3-(Dimethylamino)azetidin-1-yl][(7S)-4-{[6-(propan-2-yloxy)-1H-indazol-5-yl]amino}-5,6,7,8-tetrahydro[1]benzothieno[2,3-d]pyrimidin-7-yl]methanone

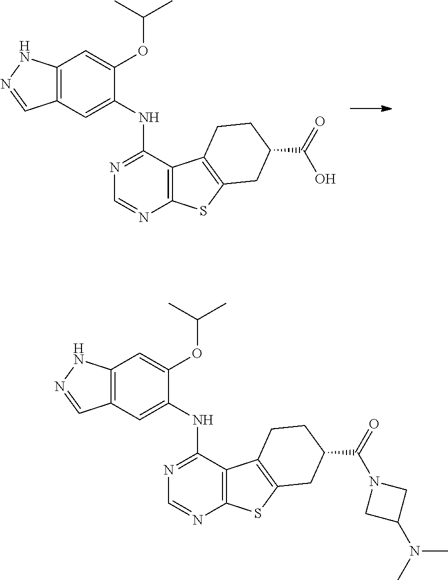 [SMILES: CC(C)Oc1cc2[nH]ncc2cc1Nc1ncnc2sc3c(c12)CC[C@H](C(=O)O)C3 ; CC(C)Oc1cc2[nH]ncc2cc1Nc1ncnc2sc3c(c12)CC[C@H](C(=O)N1CC(N(C)C)C1)C3]

50 mg (118 μmol) (7S)-4-[(6-isopropoxy-1H-indazol-5-yl)amino]-5,6,7,8-tetrahydro[1]benzothieno[2,3-d]pyrimidine-7-carboxylic acid (prepared according to intermediate example 78a) were transformed in analogy to example 1 using N,N-dimethylazetidin-3-amine to give after working up and purification 12.8 mg (20%) of the title compound.

¹H-NMR (DMSO-d6): δ=1.41 (6H), 1.86 (1H), 2.06 (1H), 2.09 (6H), 2.76-3.36 (6H), 3.66 (1H), 3.88 (1H), 3.97-4.09 (1H), 4.24 (1H), 4.88 (1H), 7.11 (1H), 7.98 (1H), 8.36 (1H), 8.52 (1H), 9.01-9.08 (1H), 12.75 (1H) ppm.

### Example 220

### 3-Azabicyclo[3.1.0]hex-3-yl{(7S)-4-[(6-fluoro-1H-indazol-5-yl)amino]-5,6,7,8-tetrahydro[1]benzothieno[2,3-d]pyrimidin-7-yl}methanone

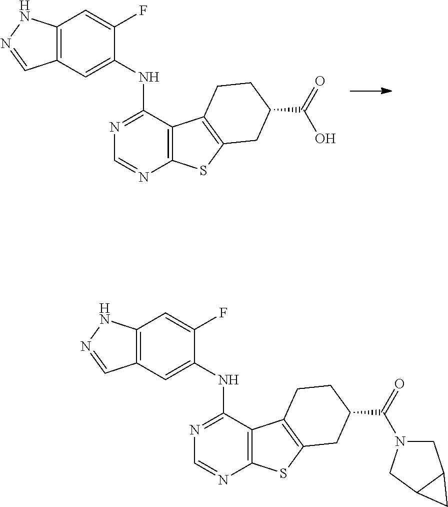 [SMILES: O=C(O)[C@H]1CCc2c(sc3ncnc(Nc4cc5cn[nH]c5cc4F)c23)C1 ; O=C([C@H]1CCc2c(sc3ncnc(Nc4cc5cn[nH]c5cc4F)c23)C1)N1CC2CC2C1]

100 mg (261 μmol) (7S)-4-[(6-fluoro-1H-indazol-5-yl)amino]-5,6,7,8-tetrahydro[1]benzothieno[2,3-d]pyrimidine-7-carboxylic acid (prepared according to intermediate example 220a) were transformed in analogy to example 205 using 3-azabicyclo[3.1.0]hexane to give after working up and purification 69.4 mg (59%) of the title compound.

¹H-NMR (DMSO-d6): δ=0.11 (1H), 0.70 (1H), 1.55 (1H), 1.62 (1H), 1.77 (1H), 1.74 (1H), 2.01+2.08 (1H), 2.86-2.96 (3H), 3.13 (1H), 3.26 (1H), 3.60-3.77 (3H), 7.43 (1H), 8.02 (1H), 8.09 (1H), 8.18 (1H), 8.26 (1H), 13.12 (1H) ppm.

### Example 220a

### (7S)-4-[(6-Fluoro-1H-indazol-5-yl)amino]-5,6,7,8-tetrahydro[1]benzothieno[2,3-d]pyrimidine-7-carboxylic acid 3-Azabicyclo[3.1.0]hex-3-yl

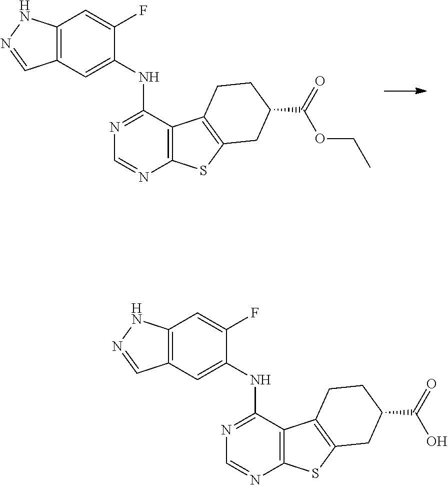 [SMILES: CCOC(=O)[C@H]1CCc2c(sc3ncnc(Nc4cc5cn[nH]c5cc4F)c23)C1 ; O=C(O)[C@H]1CCc2c(sc3ncnc(Nc4cc5cn[nH]c5cc4F)c23)C1]

1.41 g (3.43 mmol) ethyl (7S)-4-[(6-fluoro-1H-indazol-5-yl)amino]-5,6,7,8-tetrahydro[1]benzothieno[2,3-d]pyrimidine-7-carboxylate (prepared according to intermediate example 220b) were transformed in analogy to intermediate example 1a to give after working up and purification 1.28 g (94%) of the title compound.

### Example 220b

### Ethyl (7S)-4-[(6-fluoro-1H-indazol-5-yl)amino]-5,6,7,8-tetrahydro[1]benzothieno[2,3-d]pyrimidine-7-carboxylate

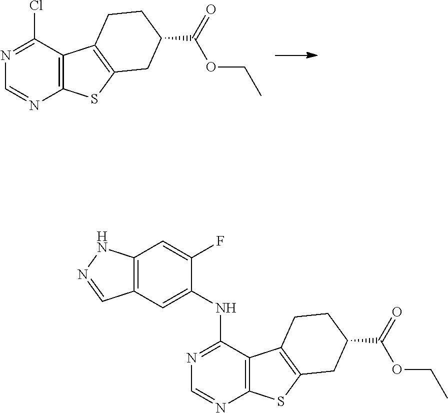 [SMILES: CCOC(=O)[C@H]1CCc2c(sc3ncnc(Cl)c23)C1 ; CCOC(=O)[C@H]1CCc2c(sc3ncnc(Nc4cc5cn[nH]c5cc4F)c23)C1]

1.30 g (4.38 mmol) ethyl (7S)-4-chloro-5,6,7,8-tetrahydro[1]benzothieno[2,3-d]pyrimidine-7-carboxylate (prepared according to intermediate example 1c) were transformed in analogy to intermediate example 1b using 6-fluoro-1H-indazol-5-amine (CAS-No: 709046-14-0) to give after working up and purification 1.41 g (76%) of the title compound.

### Example 221

### [(2R,6S)-2,6-Dimethylmorpholin-4-yl]{(7S)-4-[(6-fluoro-1H-indazol-5-yl)amino]-5,6,7,8-tetrahydro[1]benzothieno[2,3-d]pyrimidin-7-yl}methanone

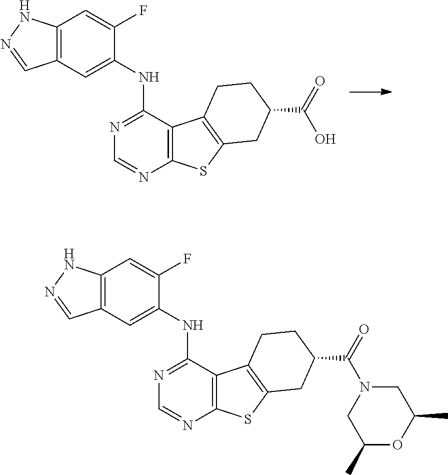 [SMILES: O=C(O)[C@H]1CCc2c(sc3ncnc(Nc4cc5cn[nH]c5cc4F)c23)C1 ; C[C@@H]1CN(C(=O)[C@H]2CCc3c(sc4ncnc(Nc5cc6cn[nH]c6cc5F)c34)C2)C[C@H](C)O1]

100 mg (261 μmol) (7S)-4-[(6-fluoro-1H-indazol-5-yl)amino]-5,6,7,8-tetrahydro[1]benzothieno[2,3-d]pyrimidine-7-carboxylic acid (prepared according to intermediate example 220a) were transformed in analogy to example 1 using (2R,6S)-2,6-dimethylmorpholine (CAS-No: 6485-55-8) to give after working up and purification 23.2 mg (17%) of the title compound.

¹H-NMR (DMSO-d6): δ=1.09-1.16 (6H), 1.73-1.92 (1H), 2.05 (1H), 2.28 (1H), 2.78 (1H), 2.87-3.06 (2H), 3.13-3.30 (3H), 3.45 (1H), 3.54 (1H), 3.99 (1H), 4.32 (1H), 7.44 (1H), 8.00+8.01 (1H), 8.11 (1H), 8.23 (1H), 8.27 (1H), 13.15 (1H) ppm.

### Example 222

### (7S)-4-[(6-Fluoro-1H-indazol-5-yl)amino]-N-(2-methoxyethyl)-N-methyl-5,6,7,8-tetrahydro[1]benzothieno[2,3-d]pyrimidine-7-carboxamide

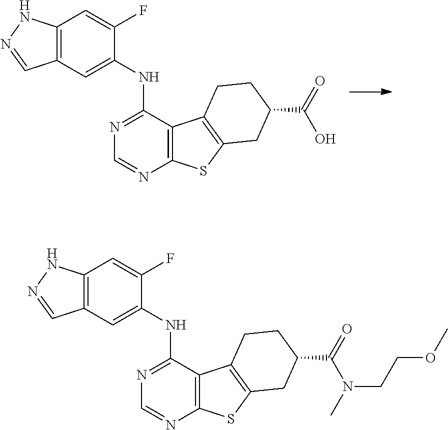 [SMILES: O=C(O)[C@H]1CCc2c(sc3ncnc(Nc4cc5cn[nH]c5cc4F)c23)C1 ; COCCN(C)C(=O)[C@H]1CCc2c(sc3ncnc(Nc4cc5cn[nH]c5cc4F)c23)C1]

100 mg (261 μmol) (7S)-4-[(6-fluoro-1H-indazol-5-yl)amino]-5,6,7,8-tetrahydro[1]benzothieno[2,3-d]pyrimidine-7-carboxylic acid (prepared according to intermediate example 220a) were transformed in analogy to example 1 using 2-methoxy-N-methylethanamine to give after working up and purification 38.1 mg (29%) of the title compound.

¹H-NMR (DMSO-d6): δ=1.81 (1H), 2.07 (1H), 2.85-3.02 (2H), 2.89+3.13 (3H), 3.10-3.33 (3H), 3.27+3.29 (3H), 3.42-3.54 (3H), 3.61 (1H), 7.45 (1H), 8.01+8.03 (1H), 8.12 (1H), 8.22 (1H), 8.27 (1H), 13.16 (1H) ppm.

### Example 223

### {(7S)-4-[(6-Fluoro-1H-indazol-5-yl)amino]-5,6,7,8-tetrahydro[1]benzothieno[2,3-d]pyrimidin-7-yl}[(3R)-3-methylmorpholin-4-yl]methanone

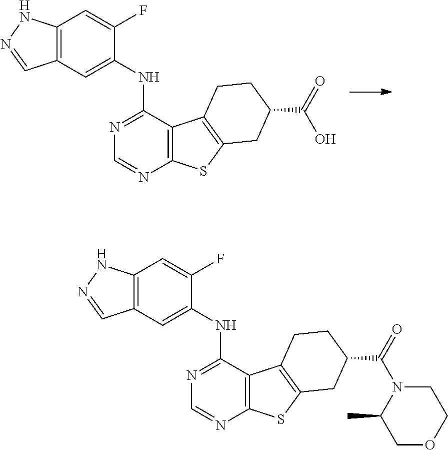 [SMILES: O=C(O)[C@H]1CCc2c(sc3ncnc(Nc4cc5cn[nH]c5cc4F)c23)C1 ; C[C@@H]1COCCN1C(=O)[C@H]1CCc2c(sc3ncnc(Nc4cc5cn[nH]c5cc4F)c23)C1]

100 mg (261 μmol) (7S)-4-[(6-fluoro-1H-indazol-5-yl)amino]-5,6,7,8-tetrahydro[1]benzothieno[2,3-d]pyrimidine-7-carboxylic acid (prepared according to intermediate example 220a) were transformed in analogy to example 1 using (3R)-3-methylmorpholine (CAS-No: 74572-04-6) to give after working up and purification 34.1 mg (25%) of the title compound.

¹H-NMR (DMSO-d6): δ=1.17+1.32 (3H), 1.89 (1H), 2.05 (1H), 2.86-3.06 (2H), 3.09-3.90 (8H), 4.10 (1H), 4.17+4.42 (1H), 7.45 (1H), 8.01+8.03 (1H), 8.11 (1H), 8.22 (1H), 8.28 (1H), 13.15 (1H) ppm.

### Example 224

### [(2R,6R)-2, 6-Dimethylmorpholin-4-yl]{(7S)-4-[(6-fluoro-1H-indazol-5-yl)amino]-5,6,7,8-tetrahydro[1]benzothieno[2,3-d]pyrimidin-7-yl}methanone

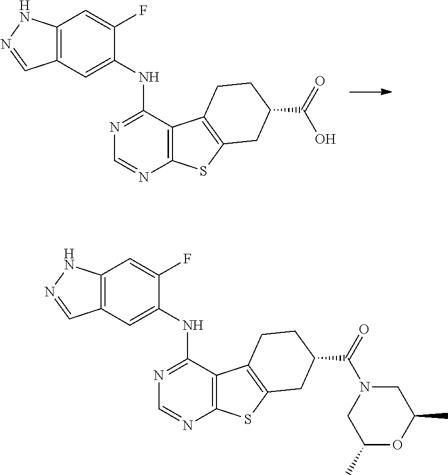 [SMILES: O=C(O)[C@H]1CCc2c(sc3ncnc(Nc4cc5cn[nH]c5cc4F)c23)C1 ; C[C@@H]1CN(C(=O)[C@H]2CCc3c(sc4ncnc(Nc5cc6cn[nH]c6cc5F)c34)C2)C[C@@H](C)O1]

100 mg (261 μmol) (7S)-4-[(6-fluoro-1H-indazol-5-yl)amino]-5,6,7,8-tetrahydro[1]benzothieno[2,3-d]pyrimidine-7-carboxylic acid (prepared according to intermediate example 220a) were transformed in analogy to example 1 using (2R,6R)-2,6-dimethylmorpholine to give after working up and purification 11.7 mg (8%) of the title compound.

¹H-NMR (DMSO-d6): δ=1.10 (3H), 1.14 (3H), 1.85 (1H), 2.07 (1H), 2.89 (1H), 3.03 (1H), 3.16-3.31 (5H), 3.55 (1H), 3.71 (1H), 3.94 (2H), 7.45 (1H), 8.00+8.02 (1H), 8.11 (1H), 8.23 (1H), 8.27 (1H), 13.15 (1H) ppm.

### Example 225

### [(2S,6S)-2,6-Dimethylmorpholin-4-yl]{(7S)-4-[(6-fluoro-1H-indazol-5-yl)amino]-5,6,7,8-tetrahydro[1]benzothieno[2,3-d]pyrimidin-7-yl}methanone

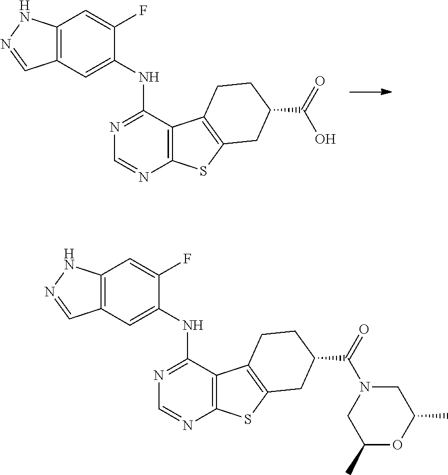 [SMILES: O=C(O)[C@H]1CCc2c(sc3ncnc(Nc4cc5cn[nH]c5cc4F)c23)C1 ; C[C@H]1CN(C(=O)[C@H]2CCc3c(sc4ncnc(Nc5cc6cn[nH]c6cc5F)c34)C2)C[C@H](C)O1]

100 mg (261 μmol) (7S)-4-[(6-fluoro-1H-indazol-5-yl)amino]-5,6,7,8-tetrahydro[1]benzothieno[2,3-d]pyrimidine-7-carboxylic acid (prepared according to intermediate example 220a) were transformed in analogy to example 1 using (2S,6S)-2,6-dimethylmorpholine to give after working up and purification 6.7 mg (5%) of the title compound.

¹H-NMR (DMSO-d6): δ=1.10 (3H), 1.13 (3H), 1.86 (1H), 2.05 (1H), 2.90-3.03 (2H), 3.14-3.41 (5H), 3.48 (1H), 3.74 (1H), 3.91-3.99 (2H), 7.44 (1H), 8.00+8.01 (1H), 8.11 (1H), 8.23 (1 h), 8.27 (1H), 13.15 (1H) ppm.

### Example 226

### {(7S)-4-[(6-Fluoro-1H-indazol-5-yl)amino]-5,6,7,8-tetrahydro[1]benzothieno[2,3-d]pyrimidin-7-yl}(morpholin-4-yl)methanone

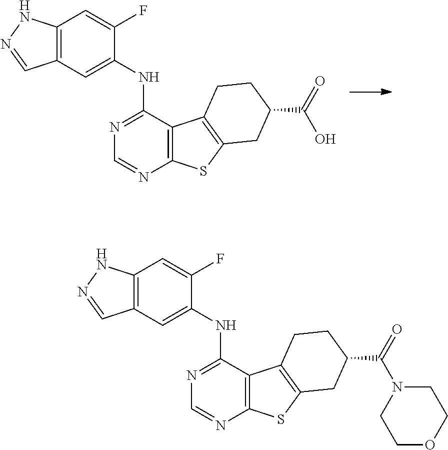 [SMILES: O=C(O)[C@H]1CCc2c(sc3ncnc(Nc4cc5cn[nH]c5cc4F)c23)C1 ; O=C([C@H]1CCc2c(sc3ncnc(Nc4cc5cn[nH]c5cc4F)c23)C1)N1CCOCC1]

100 mg (261 μmol) (7S)-4-[(6-fluoro-1H-indazol-5-yl)amino]-5,6,7,8-tetrahydro[1]benzothieno[2,3-d]pyrimidine-7-carboxylic acid (prepared according to intermediate example 220a) were transformed in analogy to example 1 using morpholine to give after working up and purification 27.0 mg (22%) of the title compound.

¹H-NMR (DMSO-d6): δ=1.83 (1H), 2.07 (1H), 2.88-3.05 (2H), 3.12-3.28 (3H), 3.44-3.68 (8H), 7.45 (1H), 8.01+8.03 (1H), 8.12 (1H), 8.22 (1H), 8.28 (1H), 13.15 (1H) ppm.

### Example 227

### {(7S)-4-[(6-Fluoro-1H-indazol-5-yl)amino]-5,6,7,8-tetrahydro[1]benzothieno[2,3-d]pyrimidin-7-yl}[(3S)-3-methylmorpholin-4-yl]methanone

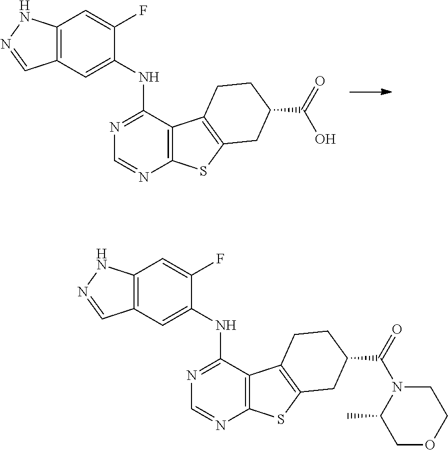 [SMILES: O=C(O)[C@H]1CCc2c(sc3ncnc(Nc4cc5cn[nH]c5cc4F)c23)C1 ; C[C@H]1COCCN1C(=O)[C@H]1CCc2c(sc3ncnc(Nc4cc5cn[nH]c5cc4F)c23)C1]

100 mg (261 μmol) (7S)-4-[(6-fluoro-1H-indazol-5-yl)amino]-5,6,7,8-tetrahydro[1]benzothieno[2,3-d]pyrimidine-7-carboxylic acid (prepared according to intermediate example 220a) were transformed in analogy to example 1 using (3S)-3-methylmorpholine (CAS-No: 350595-57-2) to give after working up and purification 14.9 mg (11%) of the title compound.

¹H-NMR (DMSO-d6): δ=1.17+1.30 (3H), 1.81 (1H), 2.05 (1H), 2.82-3.61 (8H), 3.66 (1H), 3.73-4.48 (3H), 7.45 (1H), 8.02 (1H), 8.12 (1H), 8.22 (1H), 8.28 (1H), 13.15 (1H) ppm.

### Example 228

### (7S)-4-[(6-Fluoro-1H-indazol-5-yl)amino]-N,N-dimethyl-5,6,7,8-tetrahydro[1]benzothieno[2,3-d]pyrimidine-7-carboxamide

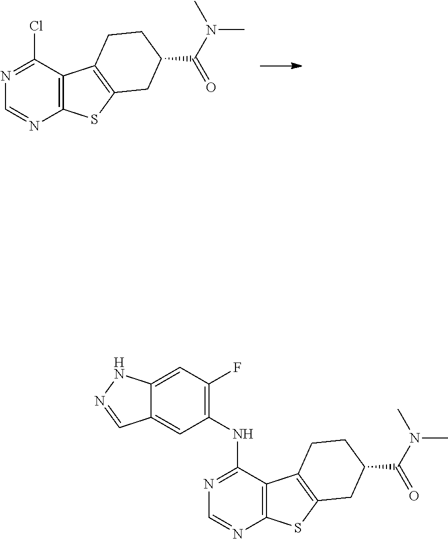 [SMILES: CN(C)C(=O)[C@H]1CCc2c(sc3ncnc(Cl)c23)C1 ; CN(C)C(=O)[C@H]1CCc2c(sc3ncnc(Nc4cc5cn[nH]c5cc4F)c23)C1]

71.8 mg (243 μmol) (7S)-4-chloro-N,N-dimethyl-5,6,7,8-tetrahydro[1]benzothieno[2,3-d]pyrimidine-7-carboxamide (prepared according to intermediate example 94a) were transformed in analogy to intermediate example 1b using 6-fluoro-1H-indazol-5-amine (CAS-No: 709046-14-0) to give after working up and purification 25.0 mg (24%) of the title compound.

¹H-NMR (DMSO-d6): δ=1.80 (1H), 2.08 (1H), 2.88 (3H), 2.90-3.02 (2H), 3.11 (3H), 3.19 (2H), 3.28 (1H), 7.45 (1H), 8.02 (1H), 8.11 (1H), 8.22 (1H), 8.28 (1H), 13.14 (1H) ppm.

### Example 229

### {(7S)-4-[(6-Chloro-1H-indazol-5-yl)amino]-5,6,7,8-tetrahydro[1]benzothieno[2,3-d]pyrimidin-7-yl}[(2S,6S)-2, 6-dimethylmorpholin-4-yl]methanone

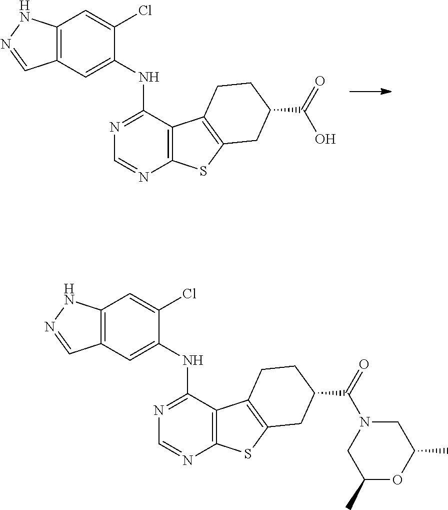 [SMILES: O=C(O)[C@H]1CCc2c(sc3ncnc(Nc4cc5cn[nH]c5cc4Cl)c23)C1 ; C[C@H]1CN(C(=O)[C@H]2CCc3c(sc4ncnc(Nc5cc6cn[nH]c6cc5Cl)c34)C2)C[C@H](C)O1]

100 mg (250 μmol) (7S)-4-[(6-chloro-1H-indazol-5-yl)amino]-5,6,7,8-tetrahydro[1]benzothieno[2,3-d]pyrimidine-7-carboxylic acid (prepared according to intermediate example 229a) were transformed in analogy to example 205 using (2S,6S)-2,6-dimethylmorpholine to give after working up and purification 111.6 mg (83%) of the title compound.

¹H-NMR (DMSO-d6): δ=1.12 (6H), 1.87 (1H), 2.07 (1H), 2.96 (2H), 3.14-3.41 (6H), 3.47 (1H), 3.94 (2H), 7.77 (1H), 8.11 (1H), 8.14 (1H), 8.25 (1H), 8.29 (1H), 13.21 (1H) ppm.

### Example 229a

### (7S)-4-[(6-Chloro-1H-indazol-5-yl)amino]-5,6,7,8-tetrahydro[1]benzothieno[2,3-d]pyrimidine-7-carboxylic acid

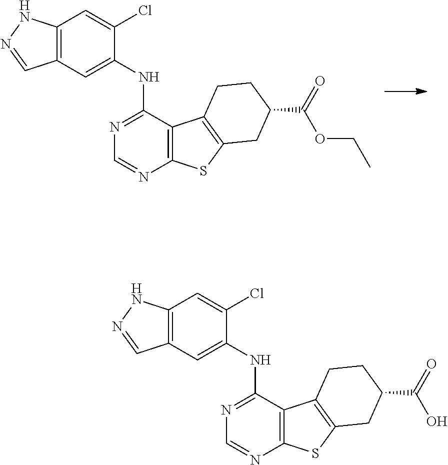 [SMILES: CCOC(=O)[C@H]1CCc2c(sc3ncnc(Nc4cc5cn[nH]c5cc4Cl)c23)C1 ; O=C(O)[C@H]1CCc2c(sc3ncnc(Nc4cc5cn[nH]c5cc4Cl)c23)C1]

1.02 g (2.38 mmol) ethyl (7S)-4-[(6-chloro-1H-indazol-5-yl)amino]-5,6,7,8-tetrahydro[1]benzothieno[2,3-d]pyrimidine-7-carboxylate (prepared according to intermediate example 229b) were transformed in analogy to intermediate example 1a to give after working up and purification 944 mg (94%) of the title compound.

### Example 229b

### Ethyl (7S)-4-[(6-chloro-1H-indazol-5-yl)amino]-5,6,7,8-tetrahydro[1]benzothieno[2,3-d]pyrimidine-7-carboxylate

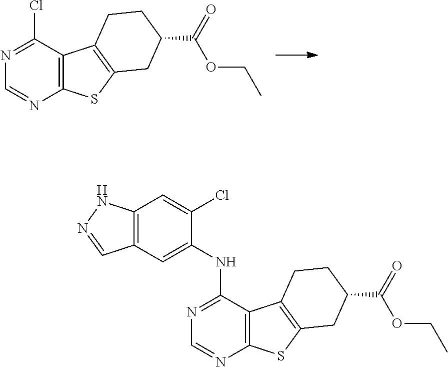 [SMILES: CCOC(=O)[C@H]1CCc2c(sc3ncnc(Cl)c23)C1 ; CCOC(=O)[C@H]1CCc2c(sc3ncnc(Nc4cc5cn[nH]c5cc4Cl)c23)C1]

1.27 g (4.27 mmol) ethyl (7S)-4-chloro-5,6,7,8-tetrahydro[1]benzothieno[2,3-d]pyrimidine-7-carboxylate (prepared according to intermediate example 1c) were transformed in analogy to intermediate example 1b using 6-chloro-1H-indazol-5-amine (CAS-No: 221681-75-0) to give after working up and purification 1.02 g (51%) of the title compound.

### Example 230

### (7S)-4-[(6-Chloro-1H-indazol-5-yl)amino]-N-(2-methoxyethyl)-N-methyl-5,6,7,8-tetrahydro[1]benzothieno[2,3-d]pyrimidine-7-carboxamide

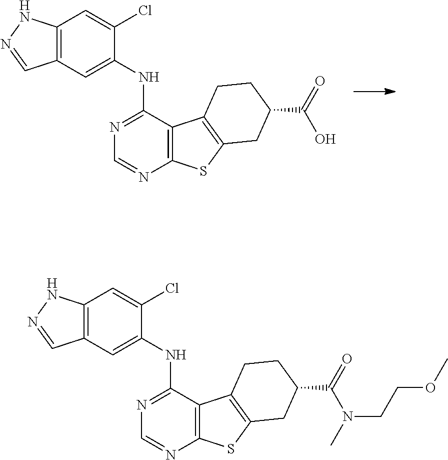 [SMILES: O=C(O)[C@H]1CCc2c(sc3ncnc(Nc4cc5cn[nH]c5cc4Cl)c23)C1 ; COCCN(C)C(=O)[C@H]1CCc2c(sc3ncnc(Nc4cc5cn[nH]c5cc4Cl)c23)C1]

87.5 mg (219 μmol) (7S)-4-[(6-chloro-1H-indazol-5-yl)amino]-5,6,7,8-tetrahydro[1]benzothieno[2,3-d]pyrimidine-7-carboxylic acid (prepared according to intermediate example 229a) were transformed in analogy to example 1 using 2-methoxy-N-methylethanamine to give after working up and purification 34.8 mg (33%) of the title compound.

¹H-NMR (DMSO-d6): δ=1.80 (1H), 2.06 (1H), 2.84-2.99 (2H), 2.87+3.12 (3H), 3.18 (2H), 3.25+3.27 (3H), 3.29 (1H), 3.42-3.54 (3H), 3.60 (1H), 7.76 (1H), 8.09-8.15 (2H), 8.22-8.28 (2H), 13.18 (1H) ppm.

### Example 231

### {(7S)-4-[(6-Chloro-1H-indazol-5-yl)amino]-5,6,7,8-tetrahydro[1]benzothieno[2,3-d]pyrimidin-7-yl}(morpholin-4-yl)methanone

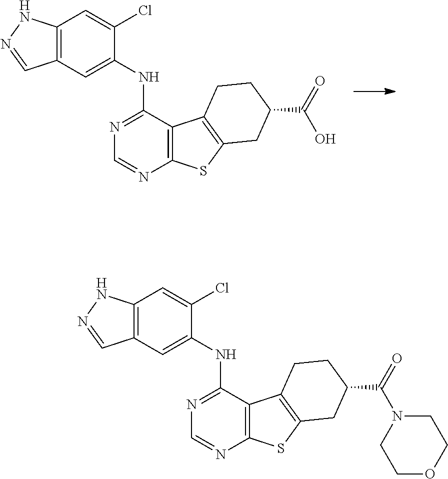 [SMILES: O=C(O)[C@H]1CCc2c(sc3ncnc(Nc4cc5cn[nH]c5cc4Cl)c23)C1 ; O=C([C@H]1CCc2c(sc3ncnc(Nc4cc5cn[nH]c5cc4Cl)c23)C1)N1CCOCC1]

87.5 mg (219 μmol) (7S)-4-[(6-chloro-1H-indazol-5-yl)amino]-5,6,7,8-tetrahydro[1]benzothieno[2,3-d]pyrimidine-7-carboxylic acid (prepared according to intermediate example 229a) were transformed in analogy to example 1 using morpholine to give after working up and purification 38.5 mg (37%) of the title compound.

¹H-NMR (DMSO-d6): δ=1.81 (1H), 2.06 (1H), 2.85-3.04 (2H), 3.18 (2H), 3.28 (1H), 3.43-3.64 (8H), 7.76 (1H), 8.11 (1H), 8.13 (1H), 8.24 (1H), 8.27 (1H), 13.20 (1H) ppm.

### Example 232

### {(7S)-4-[(6-Bromo-1H-indazol-5-yl)amino]-5,6,7,8-tetrahydro[1]benzothieno[2,3-d]pyrimidin-7-yl}[(3R)-3-methylmorpholin-4-yl]methanone

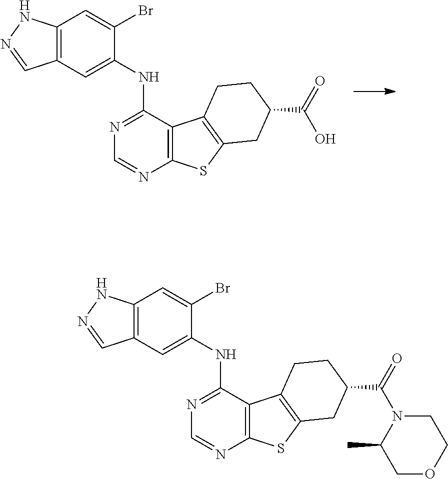 [SMILES: O=C(O)[C@H]1CCc2c(sc3ncnc(Nc4cc5cn[nH]c5cc4Br)c23)C1 ; C[C@@H]1COCCN1C(=O)[C@H]1CCc2c(sc3ncnc(Nc4cc5cn[nH]c5cc4Br)c23)C1]

100 mg (225 μmol) (7S)-4-[(6-bromo-1H-indazol-5-yl)amino]-5,6,7,8-tetrahydro[1]benzothieno[2,3-d]pyrimidine-7-carboxylic acid (prepared according to intermediate example 232a) were transformed in analogy to example 1 using (3R)-3-methylmorpholine to give after working up and purification 18.8 mg (15%) of the title compound.

¹H-NMR (DMSO-d6): δ=1.16+1.32 (3H), 1.89 (1H), 2.06 (1H), 2.87-3.59 (8H), 3.65 (1H), 3.72-4.48 (3H), 7.94 (1H), 8.06 (1H), 8.14 (1H), 8.23 (1H), 8.29 (1H), 13.22 (1H) ppm.

### Example 232a

### (7S)-4-[(6-Bromo-1H-indazol-5-yl)amino]-5,6,7,8-tetrahydro[1]benzothieno[2,3-d]pyrimidine-7-carboxylic acid

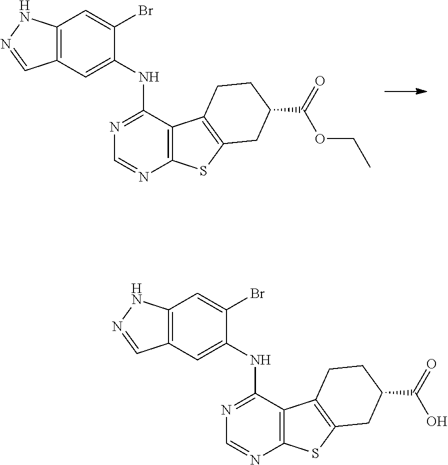 [SMILES: CCOC(=O)[C@H]1CCc2c(sc3ncnc(Nc4cc5cn[nH]c5cc4Br)c23)C1 ; O=C(O)[C@H]1CCc2c(sc3ncnc(Nc4cc5cn[nH]c5cc4Br)c23)C1]

164 mg (347 mmol) ethyl (7S)-4-[(6-bromo-1H-indazol-5-yl)amino]-5,6,7,8-tetrahydro[1]benzothieno[2,3-d]pyrimidine-7-carboxylate (prepared according to intermediate example 232b) were transformed in analogy to intermediate example 1a to give after working up and purification 149 mg (95%) of the title compound.

### Example 232b

Ethyl (7S)-4-[(6-bromo-1H-indazol-5-yl)amino]-5,6,7,8-tetrahydro[1]benzothieno[2,3-d]pyrimidine-7-carboxylate

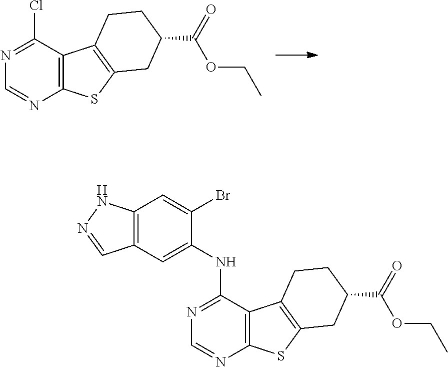 [SMILES: CCOC(=O)[C@H]1CCc2c(sc3ncnc(Cl)c23)C1 ; CCOC(=O)[C@H]1CCc2c(sc3ncnc(Nc4cc5cn[nH]c5cc4Br)c23)C1]

800 mg (2.70 mmol) ethyl (7S)-4-chloro-5,6,7,8-tetrahydro[1]benzothieno[2,3-d]pyrimidine-7-carboxylate (prepared according to intermediate example 1c) were transformed in analogy to intermediate example 1b using 6-bromo-1H-indazol-5-amine (prepared according to intermediate example 232c) to give after working up and purification 628 mg (45%) of the title compound.

### Example 232c

### 6-Bromo-1H-indazol-5-amine

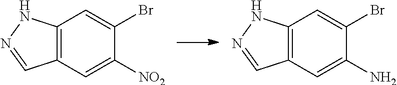 [SMILES: O=[N+]([O-])c1cc2cn[nH]c2cc1Br ; Nc1cc2cn[nH]c2cc1Br]

A mixture comprising 10 g (41.3 mmol) 6-bromo-5-nitro-1H-indazole (prepared according to the Journal of Medicinal Chemistry, 2013, vol. 56, #11 p. 4343-4356), 400 mL ethanol, 80 mL water, 23.1 g iron powder and 1.11 g ammonium chloride was stirred vigorously for 3 h at reflux. After filtration and washing with ethanol the reaction mixture was concentrated in vacuum and then taken up in ethyl acetate and washed with saturated sodium hydrogencarbonate solution and brine. The organic layer was dried over sodium sulfate, filtrated and concentrated to give 8.44 g (92%) of the title compound.

### Example 233

### {(7S)-4-[(6-Bromo-1H-indazol-5-yl)amino]-5,6,7,8-tetrahydro[1]benzothieno[2,3-d]pyrimidin-7-yl}[(2R,6R)-2, 6-dimethylmorpholin-4-yl]methanone

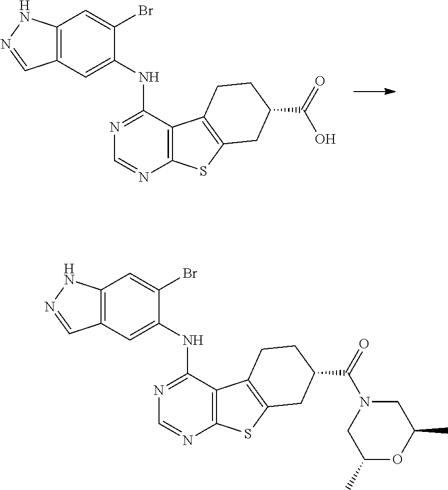 [SMILES: O=C(O)[C@H]1CCc2c(sc3ncnc(Nc4cc5cn[nH]c5cc4Br)c23)C1 ; C[C@@H]1CN(C(=O)[C@H]2CCc3c(sc4ncnc(Nc5cc6cn[nH]c6cc5Br)c34)C2)C[C@@H](C)O1]

100 mg (225 μmol) (7S)-4-[(6-bromo-1H-indazol-5-yl)amino]-5,6,7,8-tetrahydro[1]benzothieno[2,3-d]pyrimidine-7-carboxylic acid (prepared according to intermediate example 232a) were transformed in analogy to example 1 using (2R,6R)-2,6-dimethylmorpholine to give after working up and purification 11.6 mg (9%) of the title compound.

¹H-NMR (DMSO-d6): δ=1.10 (3H), 1.14 (3H), 1.85 (1H), 2.08 (1H), 2.89 (1H), 3.03 (1H), 3.18-3.37 (5H), 3.55 (1H), 3.72 (1H), 3.94 (2H), 7.94 (1H), 8.06 (1H), 8.14 (1H), 8.24 (1H), 8.30 (1H), 13.22 (1H) ppm.

### Example 234

### {(7S)-4-[(6-Bromo-1H-indazol-5-yl)amino]-5,6,7,8-tetrahydro[1]benzothieno[2,3-d]pyrimidin-7-yl}[(2R,6S)-2, 6-dimethylmorpholin-4-yl]methanone

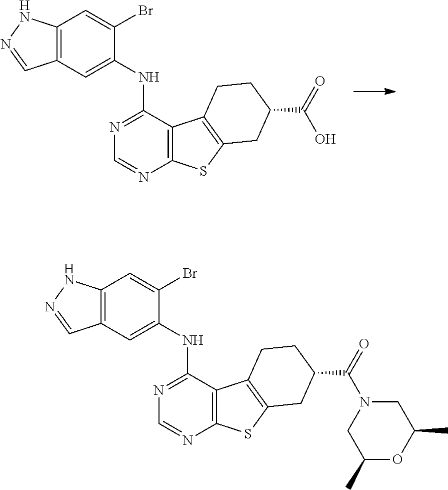 [SMILES: O=C(O)[C@H]1CCc2c(sc3ncnc(Nc4cc5cn[nH]c5cc4Br)c23)C1 ; C[C@@H]1CN(C(=O)[C@H]2CCc3c(sc4ncnc(Nc5cc6cn[nH]c6cc5Br)c34)C2)C[C@H](C)O1]

100 mg (225 μmol) (7S)-4-[(6-bromo-1H-indazol-5-yl)amino]-5,6,7,8-tetrahydro[1]benzothieno[2,3-d]pyrimidine-7-carboxylic acid (prepared according to intermediate example 232a) were transformed in analogy to example 1 using (2S,6R)-2,6-dimethylmorpholine to give after working up and purification 18.6 mg (15%) of the title compound.

¹H-NMR (DMSO-d6): δ=1.12 (6H), 1.83 (1H), 2.06 (1H), 2.28 (1H), 2.77 (1H), 2.87-3.06 (2H), 3.16-3.38 (3H), 3.45 (1H), 3.53 (1H), 4.01 (1H), 4.32 (1H), 7.94 (1H), 8.04 (1H), 8.14 (1H), 8.23 (1H), 8.30 (1H), 13.22 (1H) ppm.

### Example 235

### {(7S)-4-[(6-Bromo-1H-indazol-5-yl)amino]-5,6,7,8-tetrahydro[1]benzothieno[2,3-d]pyrimidin-7-yl}[(3S)-3-methylmorpholin-4-yl]methanone

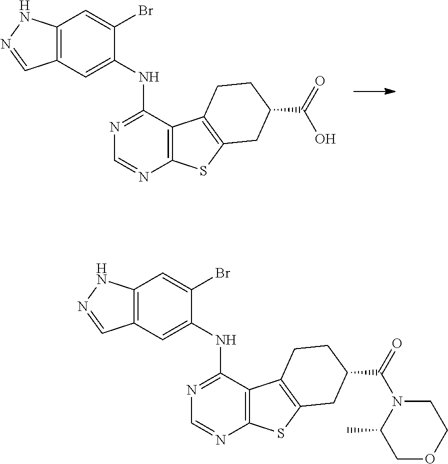 [SMILES: O=C(O)[C@H]1CCc2c(sc3ncnc(Nc4cc5cn[nH]c5cc4Br)c23)C1 ; C[C@H]1COCCN1C(=O)[C@H]1CCc2c(sc3ncnc(Nc4cc5cn[nH]c5cc4Br)c23)C1]

100 mg (225 μmol) (7S)-4-[(6-bromo-1H-indazol-5-yl)amino]-5,6,7,8-tetrahydro[1]benzothieno[2,3-d]pyrimidine-7-carboxylic acid (prepared according to intermediate example 232a) were transformed in analogy to example 1 using (3S)-3-methylmorpholine to give after working up and purification 15.4 mg (12%) of the title compound.

¹H-NMR (DMSO-d6): δ=1.17+1.30 (3H), 1.81 (1H), 2.06 (1H), 2.83-3.60 (8H), 3.66 (1H), 3.75-4.49 (3H), 7.94 (1H), 8.08 (1H), 8.14 (1H), 8.24 (1H), 8.28 (1H), 13.22 (1H) ppm.

### Example 236

### {(7S)-4-[(6-Bromo-1H-indazol-5-yl)amino]-5,6,7,8-tetrahydro[1]benzothieno[2,3-d]pyrimidin-7-yl}[(2S,6S)-2, 6-dimethylmorpholin-4-yl]methanone

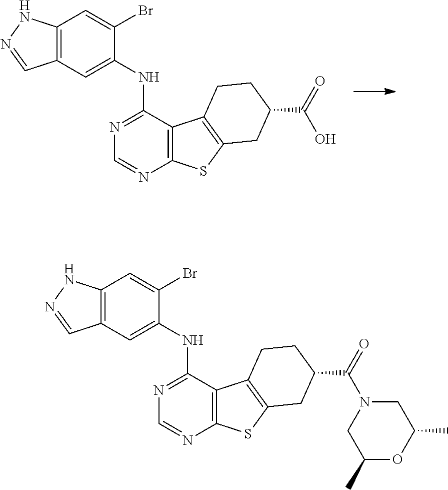 [SMILES: O=C(O)[C@H]1CCc2c(sc3ncnc(Nc4cc5cn[nH]c5cc4Br)c23)C1 ; C[C@H]1CN(C(=O)[C@H]2CCc3c(sc4ncnc(Nc5cc6cn[nH]c6cc5Br)c34)C2)C[C@H](C)O1]

100 mg (225 μmol) (7S)-4-[(6-bromo-1H-indazol-5-yl)amino]-5,6,7,8-tetrahydro[1]benzothieno[2,3-d]pyrimidine-7-carboxylic acid (prepared according to intermediate example 232a) were transformed in analogy to example 1 using (2S,6S)-2,6-dimethylmorpholine to give after working up and purification 5.4 mg (4%) of the title compound.

¹H-NMR (DMSO-d6): δ=1.12 (6H), 1.86 (1H), 2.07 (1H), 2.96 (2H), 3.14-3.42 (5H), 3.47 (1H), 3.75 (1H), 3.94 (2H), 7.94 (1H), 8.05 (1H), 8.14 (1H), 8.23 (1H), 8.30 (1H), 13.21 (1H) ppm.

### Example 237

### {(7S)-4-[(6-Bromo-1H-indazol-5-yl)amino]-5,6,7,8-tetrahydro[1]benzothieno[2,3-d]pyrimidin-7-yl}(morpholin-4-yl)methanone

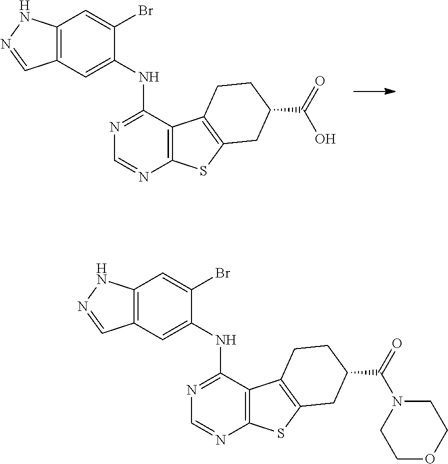 [SMILES: O=C(O)[C@H]1CCc2c(sc3ncnc(Nc4cc5cn[nH]c5cc4Br)c23)C1 ; O=C([C@H]1CCc2c(sc3ncnc(Nc4cc5cn[nH]c5cc4Br)c23)C1)N1CCOCC1]

75 mg (169 μmol) (7S)-4-[(6-bromo-1H-indazol-5-yl)amino]-5,6,7,8-tetrahydro[1]benzothieno[2,3-d]pyrimidine-7-carboxylic acid (prepared according to intermediate example 232a) were transformed in analogy to example 1 using morpholine to give after working up and purification 27.5 mg (31%) of the title compound.

¹H-NMR (DMSO-d6): δ=1.82 (1H), 2.07 (1H), 2.90 (1H), 2.98 (1H), 3.14-3.24 (2H), 3.32 (1H), 3.44-3.64 (8H), 7.92 (1H), 8.05 (1H), 8.12 (1H), 8.22 (1H), 8.26 (1H), 13.18 (1H) ppm.

### Example 238

### (7S)-4-[(6-Bromo-1H-indazol-5-yl)amino]-N-(2-methoxyethyl)-N-methyl-5,6,7,8-tetrahydro[1]benzothieno[2,3-d]pyrimidine-7-carboxamide

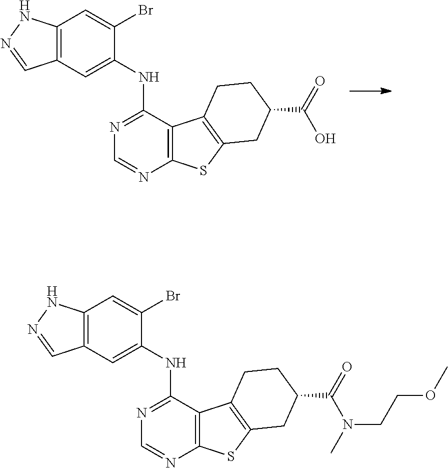 [SMILES: O=C(O)[C@H]1CCc2c(sc3ncnc(Nc4cc5cn[nH]c5cc4Br)c23)C1 ; COCCN(C)C(=O)[C@H]1CCc2c(sc3ncnc(Nc4cc5cn[nH]c5cc4Br)c23)C1]

75 mg (169 μmol) (7S)-4-[(6-bromo-1H-indazol-5-yl)amino]-5,6,7,8-tetrahydro[1]benzothieno[2,3-d]pyrimidine-7-carboxylic acid (prepared according to intermediate example 232a) were transformed in analogy to example 1 using 2-methoxy-N-methylethanamine to give after working up and purification 35.8 mg (40%) of the title compound.

¹H-NMR (DMSO-d6): δ=1.80 (1H), 2.06 (1H), 2.85-2.99 (2H), 2.87+3.12 (3H), 3.18 (2H), 3.25+3.27 (3H), 3.32 (1H), 3.41-3.54 (3H), 3.60 (1H), 7.92 (1H), 8.04+8.07 (1H), 8.12 (1H), 8.22 (1H), 8.26 (1H), 13.18 (1H) ppm.

### Example 239

### [(7S)-4-{[6-(Dimethylamino)-1H-indazol-5-yl]amino}-5,6,7,8-tetrahydro[1]benzothieno[2,3-d]pyrimidin-7-yl][(2S,6S)-2,6-dimethylmorpholin-4-yl]methanone

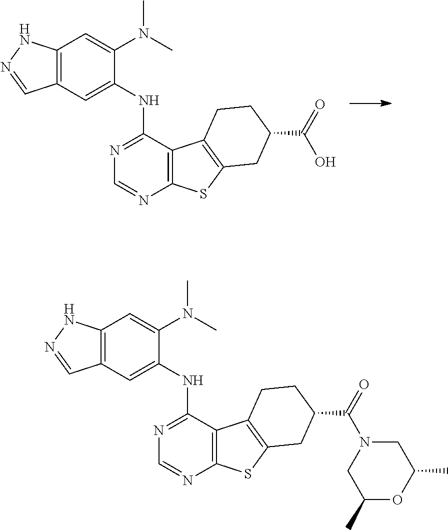 [SMILES: CN(C)c1cc2[nH]ncc2cc1Nc1ncnc2sc3c(c12)CC[C@H](C(=O)O)C3 ; C[C@H]1CN(C(=O)[C@H]2CCc3c(sc4ncnc(Nc5cc6cn[nH]c6cc5N(C)C)c34)C2)C[C@H](C)O1]

50 mg (122 μmol) (7S)-4-{[6-(dimethylamino)-1H-indazol-5-yl]amino}-5,6,7,8-tetrahydro[1]benzothieno[2,3-d]pyrimidine-7-carboxylic acid (prepared according to intermediate example 187a) were transformed in analogy to example 1 using (2S,6S)-2,6-dimethylmorpholine to give after working up and purification 34.5 mg (53%) of the title compound.

¹H-NMR (DMSO-d6): δ=1.12 (6H), 1.93 (1H), 2.15 (1H), 2.74 (6H), 2.89-3.04 (2H), 3.13-3.38 (5H), 3.48 (1H), 3.76 (1H), 3.95 (2H), 7.44 (1H), 8.05 (1H), 8.53 (1H), 9.00 (1H), 9.16 (1H), 12.91 (1H) ppm.

### Example 240

### [(7S)-4-{[6-(Dimethylamino)-1H-indazol-5-yl]amino}-5,6,7,8-tetrahydro[1]benzothieno[2,3-d]pyrimidin-7-yl][(2R,6R)-2,6-dimethylmorpholin-4-yl]methanone

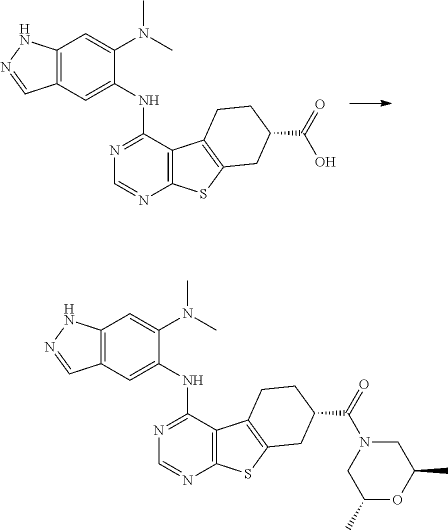 [SMILES: CN(C)c1cc2[nH]ncc2cc1Nc1ncnc2sc3c(c12)CC[C@H](C(=O)O)C3 ; C[C@@H]1CN(C(=O)[C@H]2CCc3c(sc4ncnc(Nc5cc6cn[nH]c6cc5N(C)C)c34)C2)C[C@@H](C)O1]

50 mg (122 μmol) (7S)-4-{[6-(dimethylamino)-1H-indazol-5-yl]amino}-5,6,7,8-tetrahydro[1]benzothieno[2,3-d]pyrimidine-7-carboxylic acid (prepared according to intermediate example 187a) were transformed in analogy to example 1 using (2R,6R)-2,6-dimethylmorpholine to give after working up and purification 23.2 mg (36%) of the title compound.

¹H-NMR (DMSO-d6): δ=1.13 (6H), 1.91 (1H), 2.16 (1H), 2.74 (6H), 2.87-3.06 (2H), 3.18-3.34 (5H), 3.54 (1H), 3.73 (1H), 3.95 (2H), 7.44 (1H), 8.05 (1H), 8.53 (1H), 9.00 (1H), 9.15 (1H), 12.91 (1H) ppm.

### Example 241

### (7S)-4-{[6-(Dimethylamino)-1H-indazol-5-yl]amino}-N-methyl-N-(3, 3,3-trifluoropropyl)-5,6,7,8-tetrahydro[1]benzothieno[2,3-d]pyrimidine-7-carboxamide

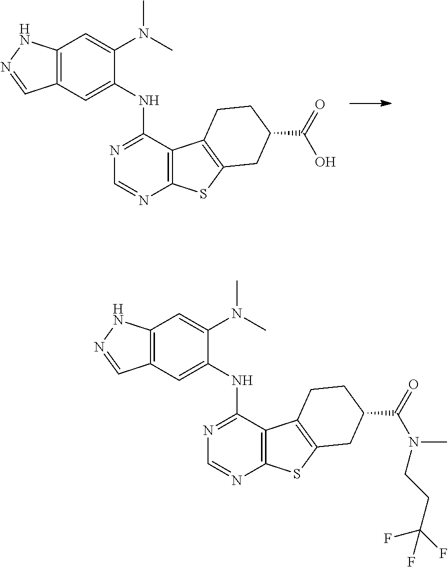 [SMILES: CN(C)c1cc2[nH]ncc2cc1Nc1ncnc2sc3c(c12)CC[C@H](C(=O)O)C3 ; CN(CCC(F)(F)F)C(=O)[C@H]1CCc2c(sc3ncnc(Nc4cc5cn[nH]c5cc4N(C)C)c23)C1]

70 mg (171 μmol) (7S)-4-{[6-(dimethylamino)-1H-indazol-5-yl]amino}-5,6,7,8-tetrahydro[1]benzothieno[2,3-d]pyrimidine-7-carboxylic acid (prepared according to intermediate example 187a) were transformed in analogy to example 1 using 3,3,3-trifluoro-N-methylpropan-1-amine (Enamine, www.enamine.net) to give after working up and purification 56.0 mg (60%) of the title compound.

¹H-NMR (DMSO-d6): δ=1.88 (1H), 2.17 (1H), 246-2.62 (2H), 2.74 (6H), 2.89+3.15 (3H), 2.95 (2H), 3.17-3.31 (3H), 3.49 (1H), 3.68 (1H), 7.44 (1H), 8.05 (1H), 8.53 (1H), 9.01 (1H), 9.16 (1H), 12.91 (1H) ppm.

### Example 242

### (7S)-4-({6-[(3-Hydroxy-3-methylbutyl)(methyl)amino]-1H-indazol-5-yl}amino)-N,N-dimethyl-5,6,7,8-tetrahydro[1]benzothieno[2,3-d]pyrimidine-7-carboxamide

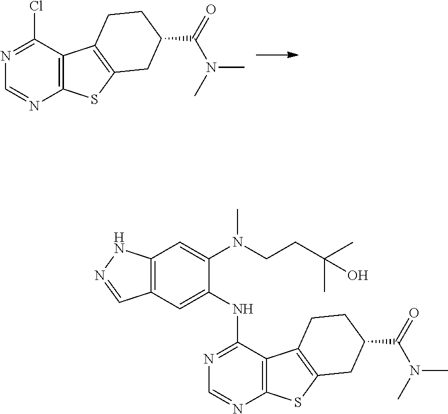 [SMILES: CN(C)C(=O)[C@H]1CCc2c(sc3ncnc(Cl)c23)C1 ; CN(C)C(=O)[C@H]1CCc2c(sc3ncnc(Nc4cc5cn[nH]c5cc4N(C)CCC(C)(C)O)c23)C1]

100 mg (338 μmol) ethyl (7S)-4-chloro-5,6,7,8-tetrahydro[1]benzothieno[2,3-d]pyrimidine-7-carboxylate (prepared according to intermediate example 1c) were transformed in analogy to intermediate example 1b using 4-[(5-amino-1H-indazol-6-yl)(methyl)amino]-2-methylbutan-2-ol (prepared according to intermediate example 242a) to give after working up and purification 9.0 mg (5%) of the title compound.

¹H-NMR (DMSO-d6): δ=1.06 (6H), 1.56 (2H), 1.87 (1H), 2.16 (1H), 2.66 (3H), 2.89 (3H), 2.96 (2H), 3.09 (2H), 3.11 (3H), 3.23 (2H), 3.32 (1H), 4.23 (1H), 7.46 (1H), 8.06 (1H), 8.54 (1H), 9.07 (1H), 9.39 (1H), 12.90 (1H) ppm.

### Example 242a

### 4-[(5-Amino-1H-indazol-6-yl)(methyl)amino]-2-methylbutan-2-ol

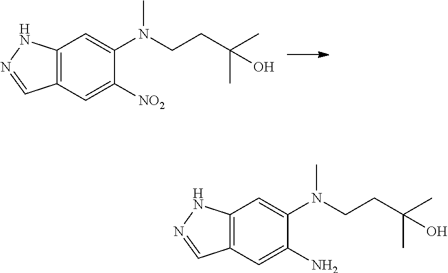 [SMILES: CN(CCC(C)(C)O)c1cc2[nH]ncc2cc1[N+](=O)[O-] ; CN(CCC(C)(C)O)c1cc2[nH]ncc2cc1N]

228 mg (821 μmol) 2-methyl-4-[methyl(5-nitro-1H-indazol-6-yl)amino]butan-2-ol (prepared according to intermediate example 242b) were transformed in analogy to intermediate example 232c using acetic acid to give after working up and purification 77.7 mg (38%) of the title compound.

### Example 242b

### 2-Methyl-4-[methyl(5-nitro-1H-indazol-6-yl)amino]butan-2-ol

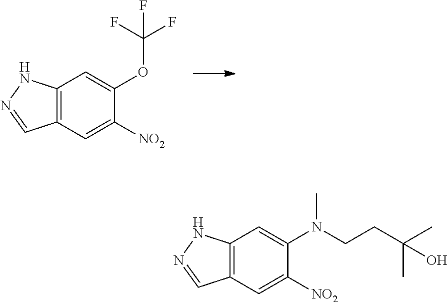 [SMILES: O=[N+]([O-])c1cc2cn[nH]c2cc1OC(F)(F)F ; CN(CCC(C)(C)O)c1cc2[nH]ncc2cc1[N+](=O)[O-]]

200 mg (809 μmol) 5-nitro-6-(trifluoromethoxy)-1H-indazole (prepared according to intermediate example 98b) were transformed in analogy to intermediate example 99b using 2-methyl-4-(methylamino)butan-2-ol to give after working up and purification 228 mg (92%) of the title compound.

### Example 243

### tert-butyl {2-[(5-{[(7S)-7-(Dimethylcarbamoyl)-5,6,7,8-tetrahydro[1]benzothieno[2,3-d]pyrimidin-4-yl]amino}-1H-indazol-6-yl)(methyl)amino]ethyl}carbamate

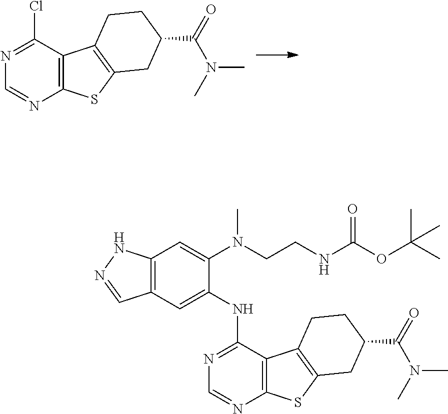 [SMILES: CN(C)C(=O)[C@H]1CCc2c(sc3ncnc(Cl)c23)C1 ; CN(C)C(=O)[C@H]1CCc2c(sc3ncnc(Nc4cc5cn[nH]c5cc4N(C)CCNC(=O)OC(C)(C)C)c23)C1]

100 mg (338 μmol) ethyl (7S)-4-chloro-5,6,7,8-tetrahydro[1]benzothieno[2,3-d]pyrimidine-7-carboxylate (prepared according to intermediate example 1c) were transformed in analogy to intermediate example 1b using tert-butyl {2-[(5-amino-1H-indazol-6-yl)(methyl)amino]ethyl}carbamate (prepared according to intermediate example 243a) to give after working up and purification 19.7 mg (10%) of the title compound.

¹H-NMR (DMSO-d6): δ=1.22 (9H), 1.85 (1H), 2.19 (1H), 2.65 (3H), 2.87 (3H), 2.93 (2H), 2.98-3.40 (7H), 3.11 (3H), 6.81 (1H), 7.45 (1H), 8.05 (1H), 8.52 (1H), 9.07 (1H), 9.24 (1H), 12.91 (1H) ppm.

### Example 243a

### tert-Butyl {2-[(5-amino-1H-indazol-6-yl)(methyl)amino]ethyl}carbamate

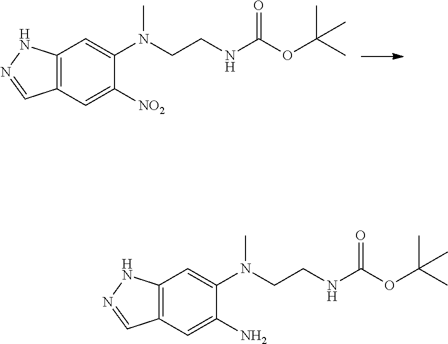 [SMILES: CN(CCNC(=O)OC(C)(C)C)c1cc2[nH]ncc2cc1[N+](=O)[O-] ; CN(CCNC(=O)OC(C)(C)C)c1cc2[nH]ncc2cc1N]

580 mg (1.73 mmol) tert-butyl {2-[methyl(5-nitro-1H-indazol-6-yl)amino]ethyl}carbamate (prepared according to intermediate example 243b) were transformed in analogy to intermediate example 94b to give after working up and purification 323 mg (61%) of the title compound.

### Example 243b

### tert-Butyl {2-[methyl(5-nitro-1H-indazol-6-yl)amino]ethyl}carbamate

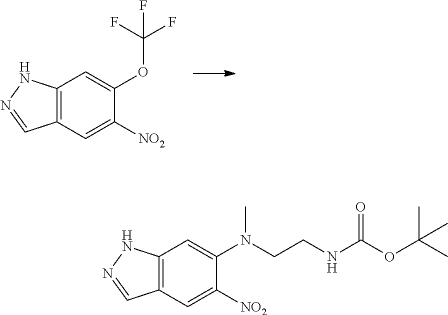 [SMILES: O=[N+]([O-])c1cc2cn[nH]c2cc1OC(F)(F)F ; CN(CCNC(=O)OC(C)(C)C)c1cc2[nH]ncc2cc1[N+](=O)[O-]]

709 mg (2.87 mmol) 5-nitro-6-(trifluoromethoxy)-1H-indazole (prepared according to intermediate example 98b) were transformed in analogy to intermediate example 99b using tert-butyl [2-(methylamino)ethyl]carbamate to give after working up and purification 586 mg (61%) of the title compound.

### Example 244

### tert-Butyl {2-[(5-{[(7S)-7-(dimethylcarbamoyl)-5,6,7,8-tetrahydro[1]benzothieno[2,3-d]pyrimidin-4-yl]amino}-1H-indazol-6-yl)amino]ethyl}carbamate

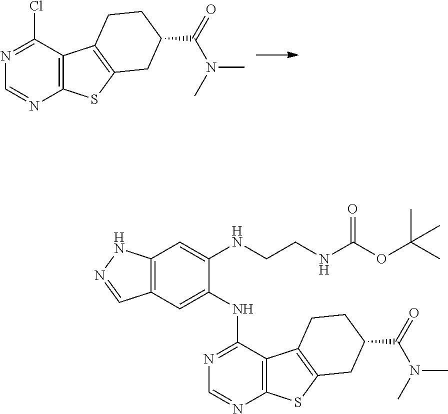 [SMILES: CN(C)C(=O)[C@H]1CCc2c(sc3ncnc(Cl)c23)C1 ; CN(C)C(=O)[C@H]1CCc2c(sc3ncnc(Nc4cc5cn[nH]c5cc4NCCNC(=O)OC(C)(C)C)c23)C1]

100 mg (338 μmol) ethyl (7S)-4-chloro-5,6,7,8-tetrahydro[1]benzothieno[2,3-d]pyrimidine-7-carboxylate (prepared according to intermediate example 1c) were transformed in analogy to intermediate example 1b using tert-butyl {2-[(5-amino-1H-indazol-6-yl)amino]ethyl}carbamate (prepared according to intermediate example 244a) to give after working up and purification 24.6 mg (13%) of the title compound.

¹H-NMR (DMSO-d6): δ=1.27 (9H), 1.76 (1H), 2.06 (1H), 2.87 (3H), 2.90 (2H), 3.08 (3H), 3.05-3.25 (6H), 3.37 (1H), 5.56 (1H), 6.53 (1H), 6.88 (1H), 7.47 (1H), 7.70 (1H), 7.78 (1H), 8.14 (1H), 12.47 (1H) ppm.

### Example 244a

### tert-Butyl {2-[(5-amino-1H-indazol-6-yl)amino]ethyl}carbamate

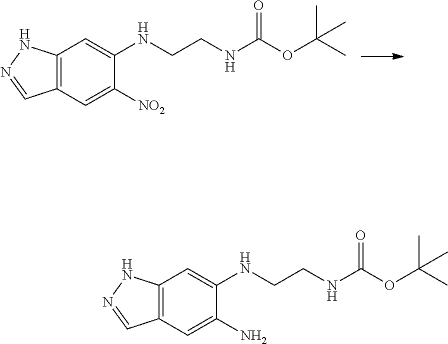 [SMILES: CC(C)(C)OC(=O)NCCNc1cc2[nH]ncc2cc1[N+](=O)[O-] ; CC(C)(C)OC(=O)NCCNc1cc2[nH]ncc2cc1N]

935 mg (2.91 mmol) tert-butyl {2-[(5-nitro-1H-indazol-6-yl)amino]ethyl}carbamate (prepared according to intermediate example 244b) were transformed in analogy to intermediate example 94b to give after working up and purification 490 mg (58%) of the title compound.

### Example 244b

### tert-Butyl {2-[(5-nitro-1H-indazol-6-yl)amino]ethyl}carbamate

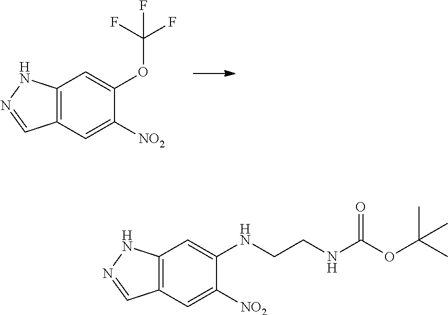 [SMILES: O=[N+]([O-])c1cc2cn[nH]c2cc1OC(F)(F)F ; CC(C)(C)OC(=O)NCCNc1cc2[nH]ncc2cc1[N+](=O)[O-]]

1.00 g (4.05 mmol) 5-nitro-6-(trifluoromethoxy)-1H-indazole (prepared according to intermediate example 98b) were transformed in analogy to intermediate example 99b using tert-butyl (2-aminoethyl)carbamate to give after working up and purification 940 mg (72%) of the title compound.

### Example 245

### (7S)—N-Methyl-4-{[6-(pyrrolidin-1-yl)-1H-indazol-5-yl]amino}-N-(3,3,3-trifluoropropyl)-5,6,7,8-tetrahydro[1]benzothieno[2,3-d]pyrimidine-7-carboxamide

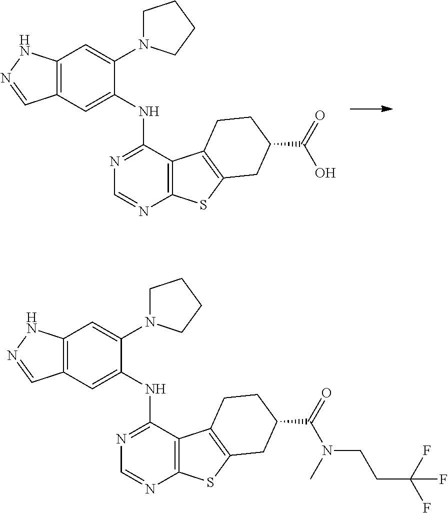 [SMILES: O=C(O)[C@H]1CCc2c(sc3ncnc(Nc4cc5cn[nH]c5cc4N4CCCC4)c23)C1 ; CN(CCC(F)(F)F)C(=O)[C@H]1CCc2c(sc3ncnc(Nc4cc5cn[nH]c5cc4N4CCCC4)c23)C1]

70 mg (161 μmol) (7S)-4-{[6-(pyrrolidin-1-yl)-1H-indazol-5-yl]amino}-5,6,7,8-tetrahydro[1]benzothieno[2,3-d]pyrimidine-7-carboxylic acid (prepared according to intermediate example 183a) were transformed in analogy to example 1 using 3,3,3-trifluoro-N-methylpropan-1-amine to give after working up and purification 41.1 mg (45%) of the title compound.

¹H-NMR (DMSO-d6): δ=1.87 (1H), 1.94 (4H), 2.08 (1H), 2.46-2.62 (2H), 2.89+3.12 (3H), 2.93 (2H), 3.07-3.31 (7H), 3.51 (1H), 3.59-3.74 (1H), 7.32 (1H), 8.00 (1H), 8.47 (1H), 8.74 (1H), 8.92 (1H), 12.81 (1H) ppm.

### Example 246

### [(2S,6S)-2,6-Dimethylmorpholin-4-yl][(7S)-4-{[6-(methylsulfanyl)-1H-indazol-5-yl]amino}-5,6,7,8-tetrahydro[1]benzothieno[2,3-d]pyrimidin-7-yl]methanone

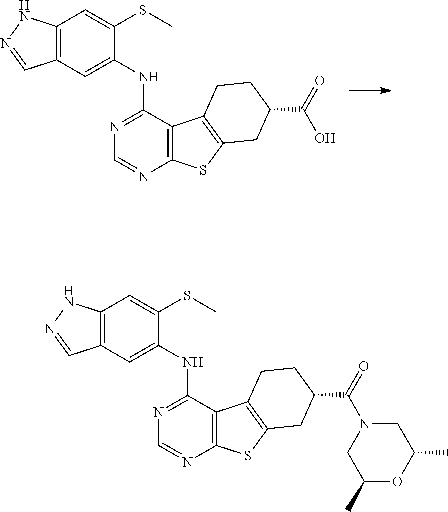 [SMILES: CSc1cc2[nH]ncc2cc1Nc1ncnc2sc3c(c12)CC[C@H](C(=O)O)C3 ; CSc1cc2[nH]ncc2cc1Nc1ncnc2sc3c(c12)CC[C@H](C(=O)N1C[C@H](C)O[C@@H](C)C1)C3]

50 mg (122 μmol) (7S)-4-{[6-(methylsulfanyl)-1H-indazol-5-yl]amino}-5,6,7,8-tetrahydro[1]benzothieno[2,3-d]pyrimidine-7-carboxylic acid (prepared according to intermediate example 246a) were transformed in analogy to example 1 using (2S,6S)-2,6-dimethylmorpholine to give after working up and purification 13.7 mg (21%) of the title compound.

¹H-NMR (DMSO-d6): δ=1.12 (6H), 1.86 (1H), 2.06 (1H), 2.45 (3H), 2.96 (2H), 3.14-3.42 (5H), 3.47 (1H), 3.75 (1H), 3.94 (2H), 7.38 (1H), 7.88 (1H), 8.05 (1H), 8.16 (1H), 8.21 (1H), 13.02 (1H) ppm.

### Example 246a

### (7S)-4-{[6-(methylsulfanyl)-1H-indazol-5-yl]amino}-5,6,7,8-tetrahydro[1]benzothieno[2,3-d]pyrimidine-7-carboxylic acid

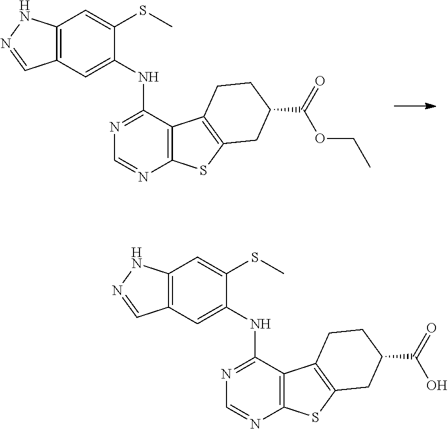 [SMILES: CCOC(=O)[C@H]1CCc2c(sc3ncnc(Nc4cc5cn[nH]c5cc4SC)c23)C1 ; CSc1cc2[nH]ncc2cc1Nc1ncnc2sc3c(c12)CC[C@H](C(=O)O)C3]

300 mg (682 μmol) ethyl (7S)-4-{[6-(methylsulfanyl)-1H-indazol-5-yl]amino}-5,6,7,8-tetrahydro[1]benzothieno[2,3-d]pyrimidine-7-carboxylate (prepared according to intermediate example 246b) were transformed in analogy to intermediate example 1a to give after working up and purification 285 mg (100%) of the title compound.

### Example 246b

### Ethyl (7S)-4-{[6-(methylsulfanyl)-1H-indazol-5-yl]amino}-5,6,7,8-tetrahydro[1]benzothieno[2,3-d]pyrimidine-7-carboxylate

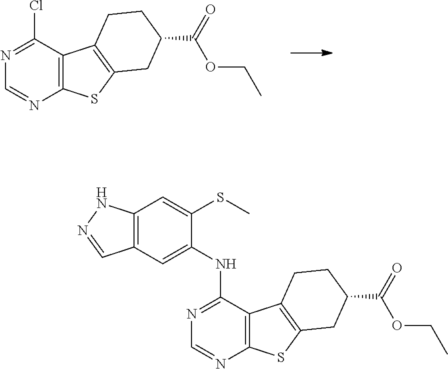 [SMILES: CCOC(=O)[C@H]1CCc2c(sc3ncnc(Cl)c23)C1 ; CCOC(=O)[C@H]1CCc2c(sc3ncnc(Nc4cc5cn[nH]c5cc4SC)c23)C1]

500 mg (1.69 mmol) ethyl (7S)-4-chloro-5,6,7,8-tetrahydro[1]benzothieno[2,3-d]pyrimidine-7-carboxylate (prepared according to intermediate example 1c) were transformed in analogy to intermediate example 1b using 6-(methylsulfanyl)-1H-indazol-5-amine (prepared according to intermediate example 106a) to give after working up and purification 501 mg (68%) of the title compound.

### Example 247

### [(2R,6R)-2, 6-Dimethylmorpholin-4-yl][(7S)-4-{[6-(methylsulfanyl)-1H-indazol-5-yl]amino}-5,6,7,8-tetrahydro[1]benzothieno[2,3-d]pyrimidin-7-yl]methanone

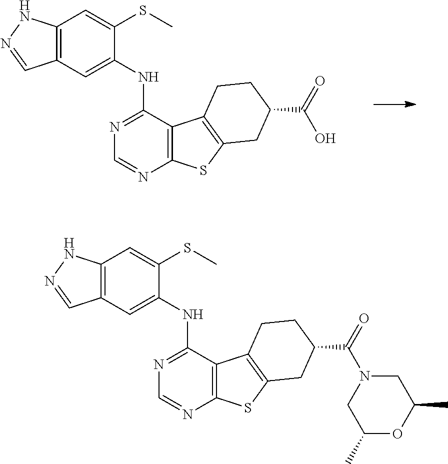 [SMILES: CSc1cc2[nH]ncc2cc1Nc1ncnc2sc3c(c12)CC[C@H](C(=O)O)C3 ; CSc1cc2[nH]ncc2cc1Nc1ncnc2sc3c(c12)CC[C@H](C(=O)N1C[C@@H](C)O[C@H](C)C1)C3]

50 mg (122 μmol) (7S)-4-{[6-(methylsulfanyl)-1H-indazol-5-yl]amino}-5,6,7,8-tetrahydro[1]benzothieno[2,3-d]pyrimidine-7-carboxylic acid (prepared according to intermediate example 246a) were transformed in analogy to example 1 using (2R,6R)-2,6-dimethylmorpholine to give after working up and purification 24.6 mg (38%) of the title compound.

¹H-NMR (DMSO-d6): δ=1.10 (6H), 1.83 (1H), 2.06 (1H), 2.43 (3H), 2.87 (1H), 3.00 (1H), 3.16-3.35 (5H), 3.53 (1H), 3.70 (1H), 3.92 (2H), 7.38 (1H), 7.89 (1H), 8.03 (1H), 8.13 (1H), 8.20 (1H), 12.99 (1H) ppm.

### Example 248

### [(3R)-3-Methylmorpholin-4-yl][(7S)-4-{[6-(methylsulfanyl)-1H-indazol-5-yl]amino}-5,6,7,8-tetrahydro[1]benzothieno[2,3-d]pyrimidin-7-yl]methanone

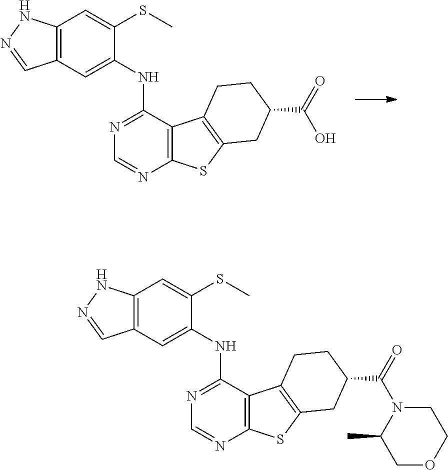 [SMILES: CSc1cc2[nH]ncc2cc1Nc1ncnc2sc3c(c12)CC[C@H](C(=O)O)C3 ; CSc1cc2[nH]ncc2cc1Nc1ncnc2sc3c(c12)CC[C@H](C(=O)N1CCOC[C@H]1C)C3]

75 mg (182 μmol) (7S)-4-{[6-(methylsulfanyl)-1H-indazol-5-yl]amino}-5,6,7,8-tetrahydro[1]benzothieno[2,3-d]pyrimidine-7-carboxylic acid (prepared according to intermediate example 246a) were transformed in analogy to example 1 using (3R)-3-methylmorpholine to give after working up and purification 53.2 mg (56%) of the title compound.

¹H-NMR (DMSO-d6): δ=1.16+1.32 (3H), 1.89 (1H), 2.05 (1H), 2.45 (3H), 2.86-3.59 (8H), 3.65 (1H), 3.72-4.48 (3H), 7.38 (1H), 7.89 (1H), 8.05 (1H), 8.15 (1H), 8.21 (1H), 13.02 (1H) ppm.

### Example 249

### (7S)—N-Methyl-4-{[6-(methylsulfanyl)-1H-indazol-5-yl]amino}-N-(3,3,3-trifluoropropyl)-5,6,7,8-tetrahydro[1]benzothieno[2,3-d]pyrimidine-7-carboxamide

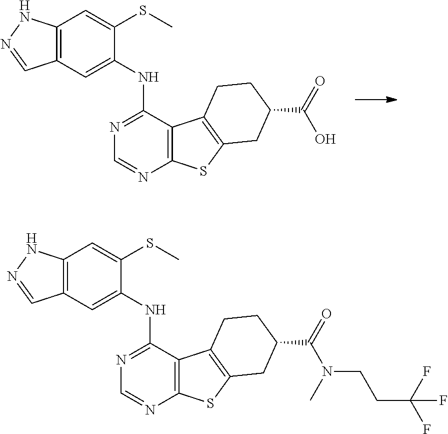 [SMILES: CSc1cc2[nH]ncc2cc1Nc1ncnc2sc3c(c12)CC[C@H](C(=O)O)C3 ; CSc1cc2[nH]ncc2cc1Nc1ncnc2sc3c(c12)CC[C@H](C(=O)N(C)CCC(F)(F)F)C3]

75 mg (182 μmol) (7S)-4-{[6-(methylsulfanyl)-1H-indazol-5-yl]amino}-5,6,7,8-tetrahydro[1]benzothieno[2,3-d]pyrimidine-7-carboxylic acid (prepared according to intermediate example 246a) were transformed in analogy to example 1 using 3,3,3-trifluoro-N-methylpropan-1-amine to give after working up and purification 37.5 mg (38%) of the title compound.

¹H-NMR (DMSO-d6): δ=1.82 (1H), 2.08 (1H), 2.45 (3H), 2.47-2.61 (2H), 2.89+3.13 (3H), 2.90-3.02 (2H), 3.20 (2H), 3.31 (1H), 3.51 (1H), 3.60-3.71 (1H), 7.39 (1H), 7.88+7.91 (1H), 8.05 (1H), 8.16 (1H), 8.22 (1H), 13.02 (1H) ppm.

### Example 250

### Azetidin-1-yl[(7S)-4-{[6-(methylsulfanyl)-1H-indazol-5-yl]amino}-5,6,7,8-tetrahydro[1]benzothieno[2,3-d]pyrimidin-7-yl]methanone

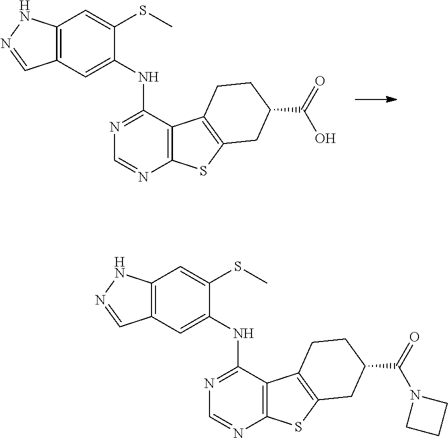 [SMILES: CSc1cc2[nH]ncc2cc1Nc1ncnc2sc3c(c12)CC[C@H](C(=O)O)C3 ; CSc1cc2[nH]ncc2cc1Nc1ncnc2sc3c(c12)CC[C@H](C(=O)N1CCC1)C3]

400 mg (972 μmol) (7S)-4-{[6-(methylsulfanyl)-1H-indazol-5-yl]amino}-5,6,7,8-tetrahydro[1]benzothieno[2,3-d]pyrimidine-7-carboxylic acid (prepared according to intermediate example 246a) were transformed in analogy to example 1 using azetidine to give after working up and purification 241 mg (52%) of the title compound.

¹H-NMR (DMSO-d6): δ=1.78 (1H), 2.07 (1H), 2.22 (2H), 2.44 (3H), 2.77 (1H), 2.90 (2H), 3.14 (1H), 3.22-3.41 (1H), 3.89 (2H), 4.25 (2H), 7.38 (1H), 7.89 (1H), 8.05 (1H), 8.16 (1H), 8.21 (1H), 13.01 (1H) ppm.

### Example 251

### (7S)—N-Methyl-4-{[6-(methylsulfanyl)-1H-indazol-5-yl]amino}-N-(propan-2-yl)-5,6,7,8-tetrahydro[1]benzothieno[2,3-d]pyrimidine-7-carboxamide

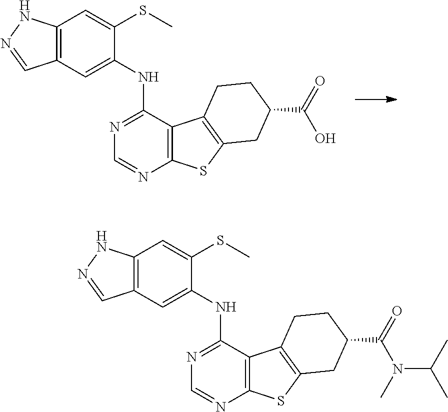 [SMILES: CSc1cc2[nH]ncc2cc1Nc1ncnc2sc3c(c12)CC[C@H](C(=O)O)C3 ; CSc1cc2[nH]ncc2cc1Nc1ncnc2sc3c(c12)CC[C@H](C(=O)N(C)C(C)C)C3]

75 mg (182 μmol) (7S)-4-{[6-(methylsulfanyl)-1H-indazol-5-yl]amino}-5,6,7,8-tetrahydro[1]benzothieno[2,3-d]pyrimidine-7-carboxylic acid (prepared according to intermediate example 246a) were transformed in analogy to example 1 using N-methylpropan-2-amine to give after working up and purification 29.6 mg (33%) of the title compound.

¹H-NMR (DMSO-d6): δ=1.05+1.17 (6H), 1.82 (1H), 2.04 (1H), 2.43 (3H), 2.70+2.90 (3H), 2.84-3.03 (2H), 3.07-3.26 (2H), 3.31 (1H), 4.28+4.72 (1H), 7.38 (1H), 7.88+7.91 (1H), 8.03 (1H), 8.12+8.14 (1H), 8.20 (1H), 12.98 (1H) ppm.

### Example 252

### [(2R,6S)-2, 6-Dimethylmorpholin-4-yl][(7S)-4-{[6-(methylsulfanyl)-1H-indazol-5-yl]amino}-5,6,7,8-tetrahydro[1]benzothieno[2,3-d]pyrimidin-7-yl]methanone

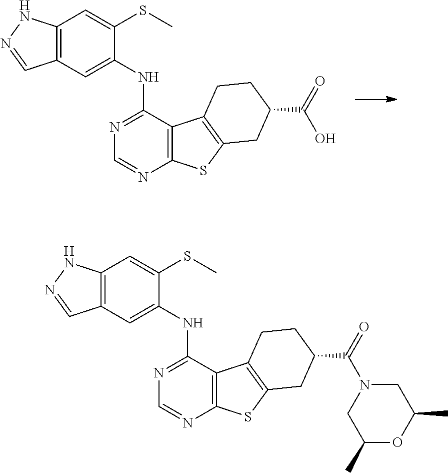 [SMILES: CSc1cc2[nH]ncc2cc1Nc1ncnc2sc3c(c12)CC[C@H](C(=O)O)C3 ; CSc1cc2[nH]ncc2cc1Nc1ncnc2sc3c(c12)CC[C@H](C(=O)N1C[C@@H](C)O[C@@H](C)C1)C3]

75 mg (182 μmol) (7S)-4-{[6-(methylsulfanyl)-1H-indazol-5-yl]amino}-5,6,7,8-tetrahydro[1]benzothieno[2,3-d]pyrimidine-7-carboxylic acid (prepared according to intermediate example 246a) were transformed in analogy to example 1 using (2R,6S)-2,6-dimethylmorpholine to give after working up and purification 31.2 mg (32%) of the title compound.

¹H-NMR (DMSO-d6): δ=1.10 (6H), 1.82 (1H), 2.04 (1H), 2.26 (1H), 2.43 (3H), 2.76 (1H), 2.86-3.03 (2H), 3.14-3.35 (3H), 3.43 (1H), 3.52 (1H), 3.99 (1H), 4.31 (1H), 7.37 (1H), 7.87 (1H), 8.03 (1H), 8.13 (1H), 8.20 (1H), 12.99 (1H) ppm.

### Example 253

### (7S)—N-(2-Methoxyethyl)-N-methyl-4-{[6-(methylsulfanyl)-1H-indazol-5-yl]amino}-5,6,7,8-tetrahydro[1]benzothieno[2,3-d]pyrimidine-7-carboxamide

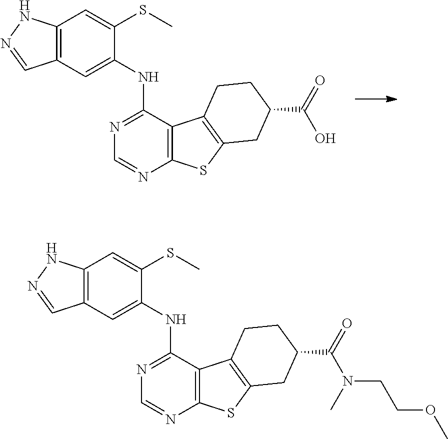 [SMILES: CSc1cc2[nH]ncc2cc1Nc1ncnc2sc3c(c12)CC[C@H](C(=O)O)C3 ; COCCN(C)C(=O)[C@H]1CCc2c(sc3ncnc(Nc4cc5cn[nH]c5cc4SC)c23)C1]

75 mg (182 μmol) (7S)-4-{[6-(methylsulfanyl)-1H-indazol-5-yl]amino}-5,6,7,8-tetrahydro[1]benzothieno[2,3-d]pyrimidine-7-carboxylic acid (prepared according to intermediate example 246a) were transformed in analogy to example 1 using 2-methoxy-N-methylethanamine to give after working up and purification 42.6 mg (46%) of the title compound.

¹H-NMR (DMSO-d6): δ=1.80 (1H), 2.06 (1H), 2.43 (3H), 2.82-3.00 (2H), 2.87+3.12 (3H), 3.19 (2H), 3.25+3.27 (3H), 3.31 (1H), 3.41-3.64 (4H), 7.38 (1H), 7.88+7.91 (1H), 8.03 (1H), 8.13 (1H), 8.20 (1H), 12.99 (1H) ppm.

### Example 254

### [(3S)-3-Methylmorpholin-4-yl][(7S)-4-{[6-(methylsulfanyl)-1H-indazol-5-yl]amino}-5,6,7,8-tetrahydro[1]benzothieno[2,3-d]pyrimidin-7-yl]methanone

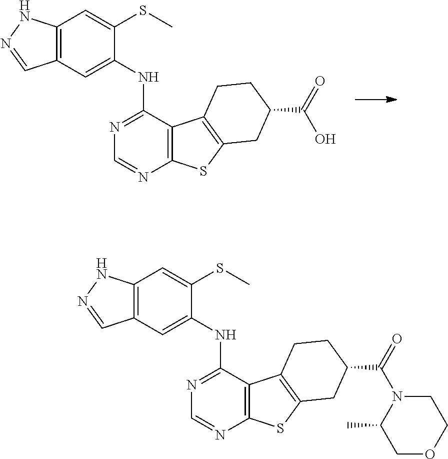 [SMILES: CSc1cc2[nH]ncc2cc1Nc1ncnc2sc3c(c12)CC[C@H](C(=O)O)C3 ; CSc1cc2[nH]ncc2cc1Nc1ncnc2sc3c(c12)CC[C@H](C(=O)N1CCOC[C@@H]1C)C3]

75 mg (182 μmol) (7S)-4-{[6-(methylsulfanyl)-1H-indazol-5-yl]amino}-5,6,7,8-tetrahydro[1]benzothieno[2,3-d]pyrimidine-7-carboxylic acid (prepared according to intermediate example 246a) were transformed in analogy to example 1 using (3S)-3-methylmorpholine to give after working up and purification 31.2 mg (33%) of the title compound.

¹H-NMR (DMSO-d6): δ=1.15+1.29 (3H), 1.78 (1H), 2.04 (1H), 2.43 (3H), 2.81-3.59 (8H), 3.64 (1H), 3.73-4.47 (3H), 7.38 (1H), 7.91 (1H), 8.03 (1H), 8.12 (1H), 8.20 (1H), 12.99 (1H) ppm.

### Example 255

### [(7S)-4-{[6-(Methylsulfanyl)-1H-indazol-5-yl]amino}-5,6,7,8-tetrahydro[1]benzothieno[2,3-d]pyrimidin-7-yl](morpholin-4-yl)methanone

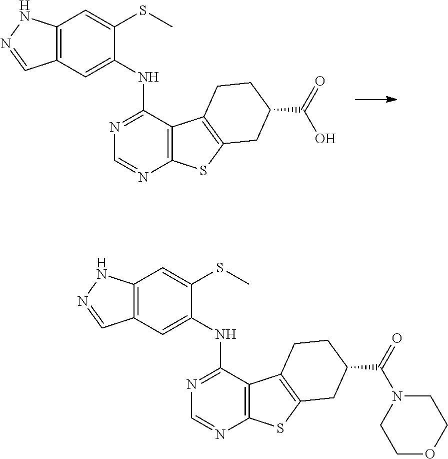 [SMILES: CSc1cc2[nH]ncc2cc1Nc1ncnc2sc3c(c12)CC[C@H](C(=O)O)C3 ; CSc1cc2[nH]ncc2cc1Nc1ncnc2sc3c(c12)CC[C@H](C(=O)N1CCOCC1)C3]

75 mg (182 μmol) (7S)-4-{[6-(methylsulfanyl)-1H-indazol-5-yl]amino}-5,6,7,8-tetrahydro[1]benzothieno[2,3-d]pyrimidine-7-carboxylic acid (prepared according to intermediate example 246a) were transformed in analogy to example 1 using morpholine to give after working up and purification 27.4 mg (30%) of the title compound.

¹H-NMR (DMSO-d6): δ=1.82 (1H), 2.06 (1H), 2.43 (3H), 2.90 (1H), 2.98 (1H), 3.13-3.25 (2H), 3.33 (1H), 3.46-3.65 (8H), 7.37 (1H), 7.89 (1H), 8.03 (1H), 8.13 (1H), 8.20 (1H), 12.99 (1H) ppm.

### Example 256

### tert-Butyl {2-[(5-{[(7S)-7-(dimethylcarbamoyl)-5,6,7,8-tetrahydro[1]benzothieno[2,3-d]pyrimidin-4-yl]amino}-1H-indazol-6-yl)oxy]ethyl}methylcarbamate

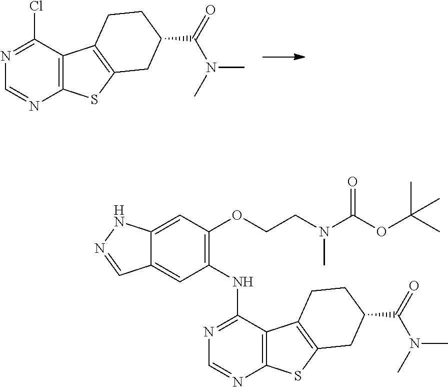 [SMILES: CN(C)C(=O)[C@H]1CCc2c(sc3ncnc(Cl)c23)C1 ; CN(C)C(=O)[C@H]1CCc2c(sc3ncnc(Nc4cc5cn[nH]c5cc4OCCN(C)C(=O)OC(C)(C)C)c23)C1]

A mixture comprising 200 mg (676 μmol) (7S)-4-chloro-N,N-dimethyl-5,6,7,8-tetrahydro[1]benzothieno[2,3-d]pyrimidine-7-carboxamide (prepared according to intermediate example 94a), 228 mg tert-butyl {2-[(5-amino-1H-indazol-6-yl)oxy]ethyl}methylcarbamate (prepared according to intermediate example 256a), 6.0 mL dimethyl sulfoxide and 353 μL N-ethyl-N-isopropylpropan-2-amine was heated at 100° C. for 2.5 days. The crude mixture was filtered and purified by chromatography to give 71.9 mg (18%) of the title compound.

¹H-NMR (DMSO-d6): δ=1.13+1.27 (9H), 1.84 (1H), 2.15 (1H), 2.83 (3H), 2.89 (3H), 2.94 (2H), 3.12 (3H), 3.19 (2H), 3.27 (1H), 3.53-3.76 (2H), 4.32 (1H), 4.41 (1H), 7.18 (1H), 8.01 (1H), 8.18+8.22 (1H), 8.48 (1H), 8.77+8.88 (1H), 12.85 (1H) ppm.

### Example 256a

### tert-Butyl {2-[(5-amino-1H-indazol-6-yl)oxy]ethyl}methylcarbamate

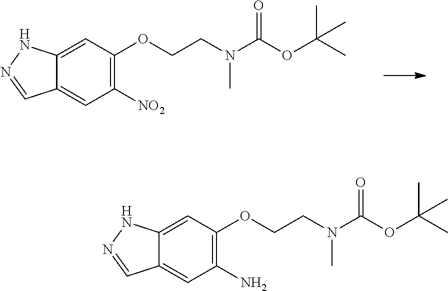 [SMILES: CN(CCOc1cc2[nH]ncc2cc1[N+](=O)[O-])C(=O)OC(C)(C)C ; CN(CCOc1cc2[nH]ncc2cc1N)C(=O)OC(C)(C)C]

1.78 mg (5.30 mmol) tert-butyl methyl{2-[(5-nitro-1H-indazol-6-yl)oxy]ethyl}carbamate (prepared according to intermediate example 256b) were transformed in analogy to intermediate example 94b to give after working up and purification 917 mg (56%) of the title compound.

### Example 256b

### tert-Butyl methyl{2-[(5-nitro-1H-indazol-6-yl)oxy]ethyl}carbamate

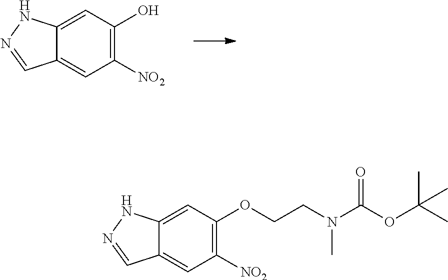 [SMILES: O=[N+]([O-])c1cc2cn[nH]c2cc1O ; CN(CCOc1cc2[nH]ncc2cc1[N+](=O)[O-])C(=O)OC(C)(C)C]

1.00 g (5.58 mmol) 5-nitro-1H-indazol-6-ol (prepared according to intermediate example 94d) were transformed in analogy to intermediate example 94c using tert-butyl (2-hydroxyethyl)methylcarbamate to give after working up and purification 2.23 g (max. 100%) of the title compound that still contains some reagent.

### Example 257

### (7S)-4-[(6-{2-[(2, 2-Dimethylpropanoyl)amino]ethoxy}-1H-indazol-5-yl)amino]-N, N-dimethyl-5,6,7,8-tetrahydro[1]benzothieno[2,3-d]pyrimidine-7-carboxamide

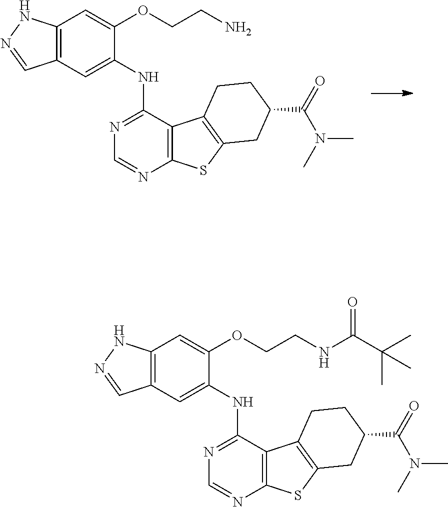 [SMILES: CN(C)C(=O)[C@H]1CCc2c(sc3ncnc(Nc4cc5cn[nH]c5cc4OCCN)c23)C1 ; CN(C)C(=O)[C@H]1CCc2c(sc3ncnc(Nc4cc5cn[nH]c5cc4OCCNC(=O)C(C)(C)C)c23)C1]

50 mg (111 μmol) (7S)-4-{[6-(2-aminoethoxy)-1H-indazol-5-yl]amino}-N,N-dimethyl-5,6,7,8-tetrahydro[1]benzothieno[2,3-d]pyrimidine-7-carboxamide (prepared according to example 108) were transformed in analogy to intermediate example 216a using 2,2-dimethylpropanoyl chloride to give after working up and purification 21.8 mg (36%) of the title compound.

¹H-NMR (DMSO-d6): δ=1.03 (9H), 1.82 (1H), 2.12 (1H), 2.89 (3H), 2.95 (2H), 3.13 (3H), 3.21 (2H), 3.27 (1H), 3.51 (1H), 3.64 (1H), 4.26 (2H), 7.12 (1H), 7.62 (1H), 8.02 (1H), 8.25 (1H), 8.51 (1H), 8.93 (1H), 12.84 (1H) ppm.

### Example 258

### Methyl {2-[(5-{[(7S)-7-(dimethylcarbamoyl)-5,6,7,8-tetrahydro[1]benzothieno[2,3-d]pyrimidin-4-yl]amino}-1H-indazol-6-yl)oxy]ethyl}carbamate

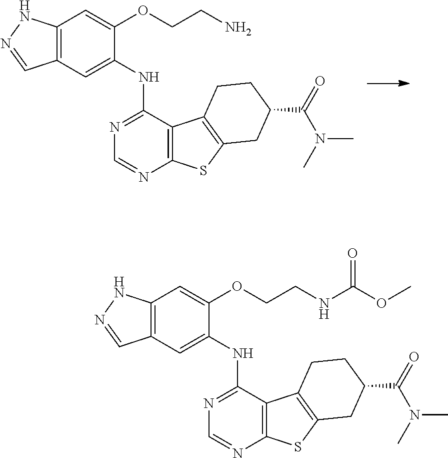 [SMILES: CN(C)C(=O)[C@H]1CCc2c(sc3ncnc(Nc4cc5cn[nH]c5cc4OCCN)c23)C1 ; COC(=O)NCCOc1cc2[nH]ncc2cc1Nc1ncnc2sc3c(c12)CC[C@H](C(=O)N(C)C)C3]

A mixture comprising 30 mg (66 μmol) (7S)-4-{[6-(2-aminoethoxy)-1H-indazol-5-yl]amino}-N,N-dimethyl-5,6,7,8-tetrahydro[1]benzothieno[2,3-d]pyrimidine-7-carboxamide (prepared according to example 108), 2.5 mL tetrahydrofurane, 9.26 μL N,N-diethylethanamine and 5.13 μL methyl carbonochloridate was stirred at 23° C. for one hour. Water and dimethylsulfoxide were added, most of the solvent removed under reduced pressure and the residue was purified by chromatography to give 8.3 mg (23%) of the title compound.

¹H-NMR (DMSO-d6): δ=1.80 (1H), 2.10 (1H), 2.89 (3H), 2.94 (2H), 3.11 (3H), 3.17 (2H), 3.26 (1H), 3.47-3.58 (2H), 3.51 (3H), 4.24 (2H), 7.10 (1H), 7.52 (1H), 8.01 (1H), 8.29 (1H), 8.53 (1H), 8.99 (1H), 12.85 (1H) ppm.

### Example 259

### tert-Butyl {(2R)-2-[(5-{[(7S)-7-(dimethylcarbamoyl)-5,6,7,8-tetrahydro[1]benzothieno[2,3-d]pyrimidin-4-yl]amino}-1H-indazol-6-yl)oxy]propyl}carbamate

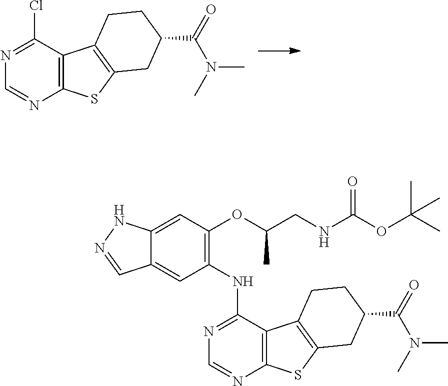 [SMILES: CN(C)C(=O)[C@H]1CCc2c(sc3ncnc(Cl)c23)C1 ; C[C@H](CNC(=O)OC(C)(C)C)Oc1cc2[nH]ncc2cc1Nc1ncnc2sc3c(c12)CC[C@H](C(=O)N(C)C)C3]

100 mg (338 μmol) (7S)-4-chloro-N,N-dimethyl-5,6,7,8-tetrahydro[1]benzothieno[2,3-d]pyrimidine-7-carboxamide (prepared according to intermediate example 94a) were transformed in analogy to intermediate example 256 using tert-butyl {(2R)-2-[(5-amino-1H-indazol-6-yl)oxy]propyl}carbamate (prepared according to intermediate example 259a) to give after working up and purification 27.5 mg (14%) of the title compound.

¹H-NMR (DMSO-d6): δ=1.30 (9H), 1.34 (3H), 1.85 (1H), 2.15 (1H), 2.89 (3H), 2.95 (2H), 3.11 (3H), 3.14-3.38 (4H), 3.47 (1H), 4.80 (1H), 7.17 (1H), 7.22 (1H), 8.01 (1H), 8.40 (1H), 8.54 (1H), 9.07 (1H), 12.80 (1H) ppm.

### Example 259a

### tert-Butyl {(2R)-2-[(5-amino-1H-indazol-6-yl)oxy]propyl}carbamate

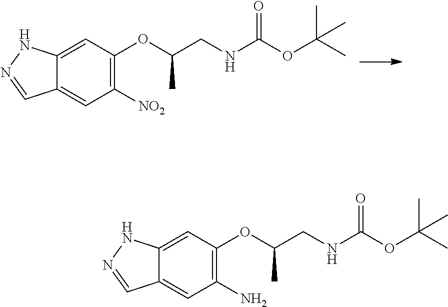 [SMILES: C[C@H](CNC(=O)OC(C)(C)C)Oc1cc2[nH]ncc2cc1[N+](=O)[O-] ; C[C@H](CNC(=O)OC(C)(C)C)Oc1cc2[nH]ncc2cc1N]

808 mg (2.40 mmol) tert-butyl {(2R)-2-[(5-nitro-1H-indazol-6-yl)oxy]propyl}carbamate (prepared according to intermediate example 259b) were transformed in analogy to intermediate example 94b to give after working up and purification 353 mg (48%) of the title compound.

### Example 259b

### tert-Butyl {(2R)-2-[(5-nitro-1H-indazol-6-yl)oxy]propyl}carbamate

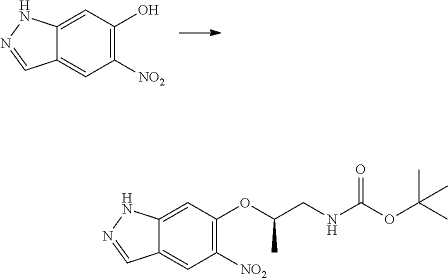 [SMILES: O=[N+]([O-])c1cc2cn[nH]c2cc1O ; C[C@H](CNC(=O)OC(C)(C)C)Oc1cc2[nH]ncc2cc1[N+](=O)[O-]]

500 mg (2.79 mmol) 5-nitro-1H-indazol-6-ol (prepared according to intermediate example 94d) were transformed in analogy to intermediate example 94c using tert-butyl [(2R)-2-hydroxypropyl]carbamate to give after working up and purification 813 mg (87%) of the title compound.

### Example 260

### Propan-2-yl {2-[(5-{[(7S)-7-(dimethylcarbamoyl)-5,6,7,8-tetrahydro[1]benzothieno[2,3-d]pyrimidin-4-yl]amino}-1H-indazol-6-yl)oxy]ethyl}carbamate

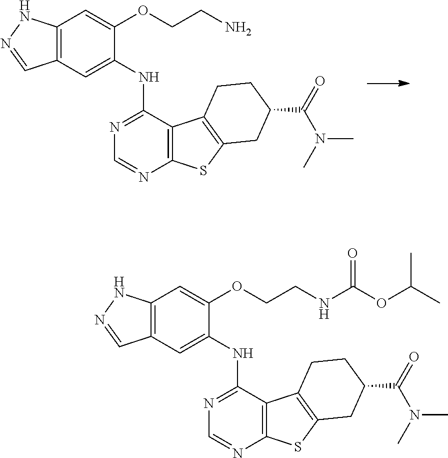 [SMILES: CN(C)C(=O)[C@H]1CCc2c(sc3ncnc(Nc4cc5cn[nH]c5cc4OCCN)c23)C1 ; CC(C)OC(=O)NCCOc1cc2[nH]ncc2cc1Nc1ncnc2sc3c(c12)CC[C@H](C(=O)N(C)C)C3]

30 mg (66 μmol) (7S)-4-{[6-(2-aminoethoxy)-1H-indazol-5-yl]amino}-N,N-dimethyl-5,6,7,8-tetrahydro[1]benzothieno[2,3-d]pyrimidine-7-carboxamide (prepared according to example 108) were transformed in analogy to example 258 using isopropyl carbonochloridate to give after working up and purification 9.2 mg (25%) of the title compound.

¹H-NMR (DMSO-d6): δ=1.11 (6H), 1.81 (1H), 2.12 (1H), 2.89 (3H), 2.95 (2H), 3.11 (3H), 3.12-3.43 (3H), 3.51 (2H), 4.23 (2H), 4.71 (1H), 7.09 (1H), 7.40 (1H), 8.01 (1H), 8.29 (1H), 8.53 (1H), 8.97 (1H), 12.85 (1H) ppm.

### Example 261

### Propan-2-yl {(2R)-2-[(5-{[(7S)-7-(dimethylcarbamoyl)-5,6,7,8-tetrahydro[1]benzothieno[2,3-d]pyrimidin-4-yl]amino}-1H-indazol-6-yl)oxy]propyl}carbamate

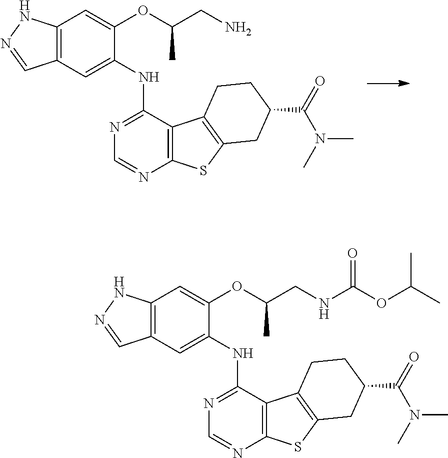 [SMILES: C[C@H](CN)Oc1cc2[nH]ncc2cc1Nc1ncnc2sc3c(c12)CC[C@H](C(=O)N(C)C)C3 ; CC(C)OC(=O)NC[C@@H](C)Oc1cc2[nH]ncc2cc1Nc1ncnc2sc3c(c12)CC[C@H](C(=O)N(C)C)C3]

A mixture comprising 50 mg (107 μmol) (7S)-4-[(6-{[(2R)-1-aminopropan-2-yl]oxy}-1H-indazol-5-yl)amino]-N,N-dimethyl-5,6,7,8-tetrahydro[1]benzothieno[2,3-d]pyrimidine-7-carboxamide (prepared according to example 152), 4.0 mL N,N-dimethylacetamide, 107 μL isopropyl carbonochloridate and 15 μL N,N-diethylethanamine was stirred at 23° C. for 1.5 hours. Water was added, the solvents removed and the crude mixture was purified by chromatography to give 32.3 mg (52%) of the title compound.

¹H-NMR (DMSO-d6): δ=1.07 (6H), 1.34 (3H), 1.84 (1H), 2.11 (1H), 2.87 (3H), 2.93 (2H), 3.09 (3H), 3.18 (2H), 3.26-3.36 (2H), 3.44 (1H), 4.67 (1H), 4.78 (1H), 7.17 (1H), 7.39 (1H), 7.99 (1H), 8.36 (1H), 8.52 (1H), 9.05 (1H), 12.79 (1H) ppm.

### Example 262

### Ethyl {2-[(5-{[(7S)-7-(dimethylcarbamoyl)-5,6,7,8-tetrahydro[1]benzothieno[2,3-d]pyrimidin-4-yl]amino}-1H-indazol-6-yl)oxy]ethyl}carbamate

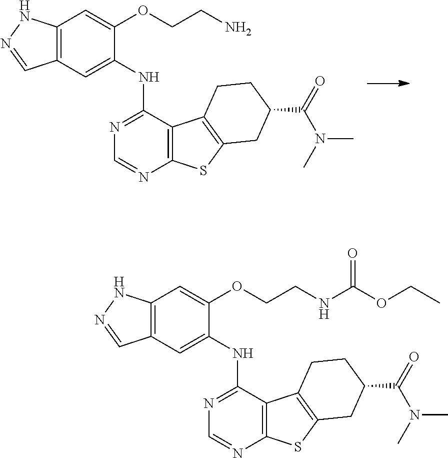 [SMILES: CN(C)C(=O)[C@H]1CCc2c(sc3ncnc(Nc4cc5cn[nH]c5cc4OCCN)c23)C1 ; CCOC(=O)NCCOc1cc2[nH]ncc2cc1Nc1ncnc2sc3c(c12)CC[C@H](C(=O)N(C)C)C3]

30 mg (66 μmol) (7S)-4-{[6-(2-aminoethoxy)-1H-indazol-5-yl]amino}-N,N-dimethyl-5,6,7,8-tetrahydro[1]benzothieno[2,3-d]pyrimidine-7-carboxamide (prepared according to intermediate example 108) were transformed in analogy to example 261 using ethyl carbonochloridate to give after working up and purification 8.5 mg (23%) of the title compound.

¹H-NMR (DMSO-d6): δ=1.11 (3H), 1.82 (1H), 2.11 (1H), 2.89 (3H), 2.95 (2H), 3.11 (3H), 3.17 (2H), 3.28 (1H), 3.52 (2H), 3.97 (2H), 4.24 (2H), 7.10 (1H), 7.47 (1H), 8.01 (1H), 8.29 (1H), 8.53 (1H), 8.98 (1H), 12.85 (1H) ppm.

### Example 263

### Ethyl {(2R)-2-[(5-{[(7S)-7-(dimethylcarbamoyl)-5,6,7,8-tetrahydro[1]benzothieno[2,3-d]pyrimidin-4-yl]amino}-1H-indazol-6-yl)oxy]propyl}carbamate

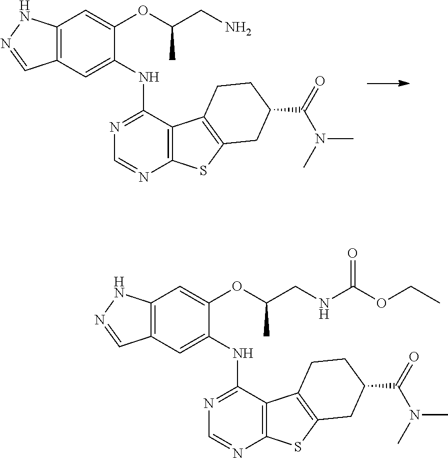 [SMILES: C[C@H](CN)Oc1cc2[nH]ncc2cc1Nc1ncnc2sc3c(c12)CC[C@H](C(=O)N(C)C)C3 ; CCOC(=O)NC[C@@H](C)Oc1cc2[nH]ncc2cc1Nc1ncnc2sc3c(c12)CC[C@H](C(=O)N(C)C)C3]

50 mg (107 μmol) (7S)-4-[(6-{[(2R)-1-aminopropan-2-yl]oxy}-1H-indazol-5-yl)amino]-N,N-dimethyl-5,6,7,8-tetrahydro[1]benzothieno[2,3-d]pyrimidine-7-carboxamide (prepared according to intermediate example 152) were transformed in analogy to example 261 using ethyl carbonochloridate to give after working up and purification 9.3 mg (15%) of the title compound.

¹H-NMR (DMSO-d6): δ=1.08 (3H), 1.36 (3H), 1.85 (1H), 2.12 (1H), 2.89 (3H), 2.95 (2H), 3.11 (3H), 3.19 (2H), 3.34 (2H), 3.44 (1H), 3.93 (2H), 4.80 (1H), 7.20 (1H), 7.50 (1H), 8.01 (1H), 8.37 (1H), 8.54 (1H), 9.07 (1H), 12.82 (1H) ppm.

### Example 264

### Methyl {(2R)-2-[(5-{[(7S)-7-(dimethylcarbamoyl)-5,6,7,8-tetrahydro[1]benzothieno[2,3-d]pyrimidin-4-yl]amino}-1H-indazol-6-yl)oxy]propyl}carbamate

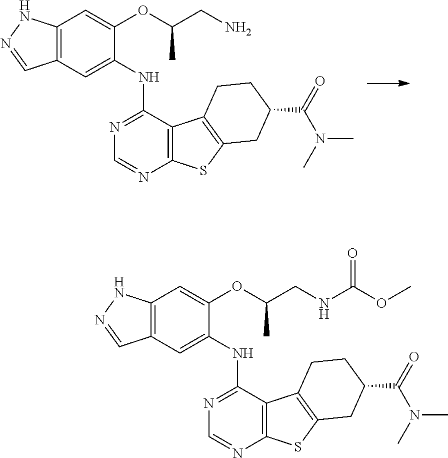 [SMILES: C[C@H](CN)Oc1cc2[nH]ncc2cc1Nc1ncnc2sc3c(c12)CC[C@H](C(=O)N(C)C)C3 ; COC(=O)NC[C@@H](C)Oc1cc2[nH]ncc2cc1Nc1ncnc2sc3c(c12)CC[C@H](C(=O)N(C)C)C3]

50 mg (107 μmol) (7S)-4-[(6-{[(2R)-1-aminopropan-2-yl]oxy}-1H-indazol-5-yl)amino]-N,N-dimethyl-5,6,7,8-tetrahydro[1]benzothieno[2,3-d]pyrimidine-7-carboxamide (prepared according to intermediate example 152) were transformed in analogy to example 261 using methyl carbonochloridate to give after working up and purification 29.7 mg (50%) of the title compound.

¹H-NMR (DMSO-d6): δ=1.35 (3H), 1.85 (1H), 2.10 (1H), 2.89 (3H), 2.95 (2H), 3.11 (3H), 3.20 (2H), 3.34 (2H), 3.42 (1H), 3.50 (3H), 4.80 (1H), 7.20 (1H), 7.54 (1H), 8.01 (1H), 8.36 (1H), 8.54 (1H), 9.06 (1H), 12.83 (1H) ppm.

### Example 265

### tert-Butyl 3-[(5-{[(7S)-7-(dimethylcarbamoyl)-5,6,7,8-tetrahydro[1]benzothieno[2,3-d]pyrimidin-4-yl]amino}-1H-indazol-6-yl)oxy]azetidine-1-carboxylate

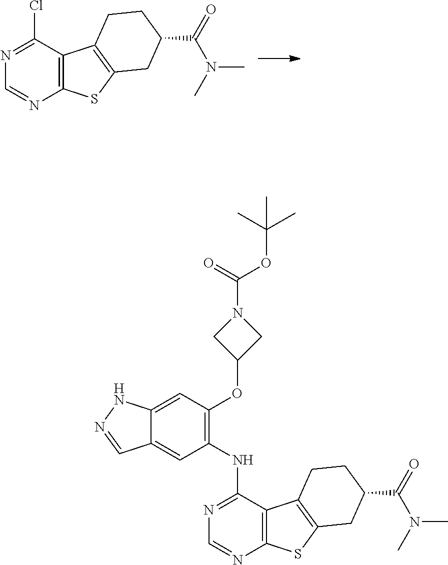 [SMILES: CN(C)C(=O)[C@H]1CCc2c(sc3ncnc(Cl)c23)C1 ; CN(C)C(=O)[C@H]1CCc2c(sc3ncnc(Nc4cc5cn[nH]c5cc4OC4CN(C(=O)OC(C)(C)C)C4)c23)C1]

100 mg (338 μmol) (7S)-4-chloro-N,N-dimethyl-5,6,7,8-tetrahydro[1]benzothieno[2,3-d]pyrimidine-7-carboxamide (prepared according to intermediate example 94a) were transformed in analogy to intermediate example 1b using tert-butyl 3-[(5-amino-1H-indazol-6-yl)oxy]azetidine-1-carboxylate (prepared according to intermediate example 265a) to give after working up and purification 22.8 mg (11%) of the title compound.

¹H-NMR (DMSO-d6): δ=1.37 (9H), 1.84 (1H), 2.11 (1H), 2.87 (3H), 2.93 (2H), 3.03-3.27 (3H), 3.10 (3H), 3.89 (2H), 4.37 (2H), 5.25 (1H), 6.87 (1H), 8.02 (1H), 8.27 (1H), 8.50 (1H), 8.95 (1H), 12.82 (1H) ppm.

### Example 265a

### tert-Butyl 3-[(5-amino-1H-indazol-6-yl)oxy]azetidine-1-carboxylate

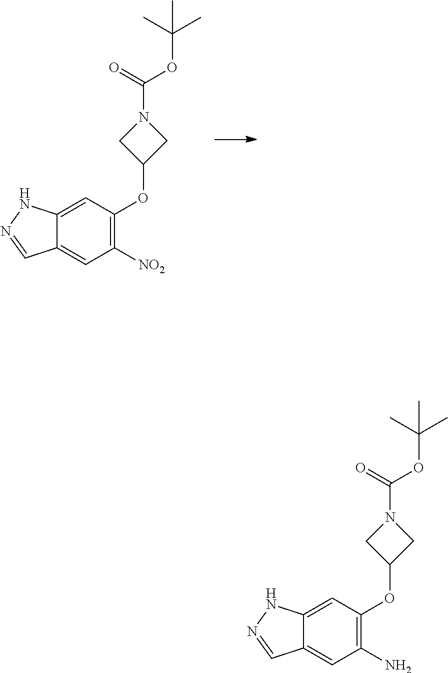 [SMILES: CC(C)(C)OC(=O)N1CC(Oc2cc3[nH]ncc3cc2[N+](=O)[O-])C1 ; CC(C)(C)OC(=O)N1CC(Oc2cc3[nH]ncc3cc2N)C1]

507 mg (1.52 mmol) (prepared according to intermediate example 265b) were transformed in analogy to example 108 using a mixture of ethanol and tetrahydrofuran as solvent to give after working up and purification 447 mg (87%) of the title compound.

### Example 265b

### tert-butyl 3-[(5-nitro-1H-indazol-6-yl)oxy]azetidine-1-carboxylate

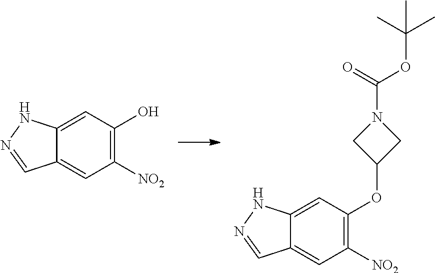 [SMILES: O=[N+]([O-])c1cc2cn[nH]c2cc1O ; CC(C)(C)OC(=O)N1CC(Oc2cc3[nH]ncc3cc2[N+](=O)[O-])C1]

1.00 g (5.58 mmol) 5-nitro-1H-indazol-6-ol (prepared according to intermediate example 94d) were transformed in analogy to intermediate example 94c using tert-butyl 3-hydroxyazetidine-1-carboxylate to give after working up and purification 507 mg (27%) of the title compound.

### Example 266

### tert-Butyl 3-[(5-{[(7S)-7-{[(2R,6S)-2, 6-dimethylmorpholin-4-yl]carbonyl}-5,6,7,8-tetrahydro[1]benzothieno[2,3-d]pyrimidin-4-yl]amino}-1H-indazol-6-yl)oxy]azetidine-1-carboxylate

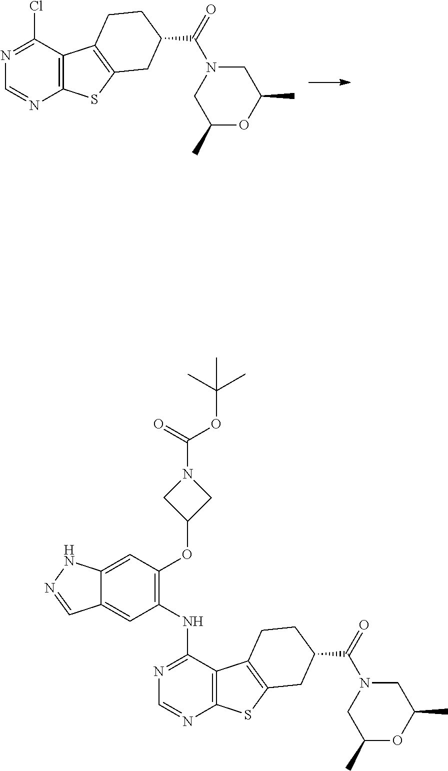 [SMILES: C[C@@H]1CN(C(=O)[C@H]2CCc3c(sc4ncnc(Cl)c34)C2)C[C@H](C)O1 ; C[C@@H]1CN(C(=O)[C@H]2CCc3c(sc4ncnc(Nc5cc6cn[nH]c6cc5OC5CN(C(=O)OC(C)(C)C)C5)c34)C2)C[C@H](C)O1]

100 mg (273 μmol) [(7S)-4-chloro-5,6,7,8-tetrahydro[1]benzothieno[2,3-d]pyrimidin-7-yl][(2R,6S)-2,6-dimethylmorpholin-4-yl]methanone (prepared according to intermediate example 266a) were transformed in analogy to intermediate example 1b using tert-butyl 3-[(5-amino-1H-indazol-6-yl)oxy]azetidine-1-carboxylate (prepared according to intermediate example 265a) to give after working up and purification 19.0 mg (11%) of the title compound.

¹H-NMR (DMSO-d6): δ=1.11 (6H), 1.38 (9H), 1.88 (1H), 2.09 (1H), 2.26 (1H), 2.75 (1H), 2.93 (2H), 3.16-3.60 (5H), 3.89 (2H), 4.00 (1H), 4.25-4.44 (3H), 5.25 (1H), 6.86 (1H), 8.02 (1H), 8.26 (1H), 8.50 (1H), 8.93+8.96 (1H), 12.84 (1H) ppm.

### Example 266a

### [(7S)-4-Chloro-5,6,7,8-tetrahydro[1]benzothieno[2,3-d]pyrimidin-7-yl][(2R,6S)-2,6-dimethylmorpholin-4-yl]methanone

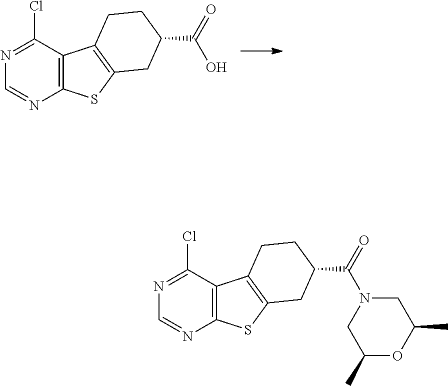 [SMILES: O=C(O)[C@H]1CCc2c(sc3ncnc(Cl)c23)C1 ; C[C@@H]1CN(C(=O)[C@H]2CCc3c(sc4ncnc(Cl)c34)C2)C[C@H](C)O1]

300 mg (1.12 mmol) (7S)-4-chloro-5,6,7,8-tetrahydro[1]benzothieno[2,3-d]pyrimidine-7-carboxylic acid (prepared according to intermediate example 66b) were transformed in analogy to example 1 using (2R,6S)-2,6-dimethylmorpholine to give after working up and purification 210 mg (51%) of the title compound.

### Example 267

### tert-Butyl 3-[(5-{[(7S)-7-{[(3S)-3-methylmorpholin-4-yl]carbonyl}-5,6,7,8-tetrahydro[1]benzothieno[2,3-d]pyrimidin-4-yl]amino}-1H-indazol-6-yl)oxy]azetidine-1-carboxylate

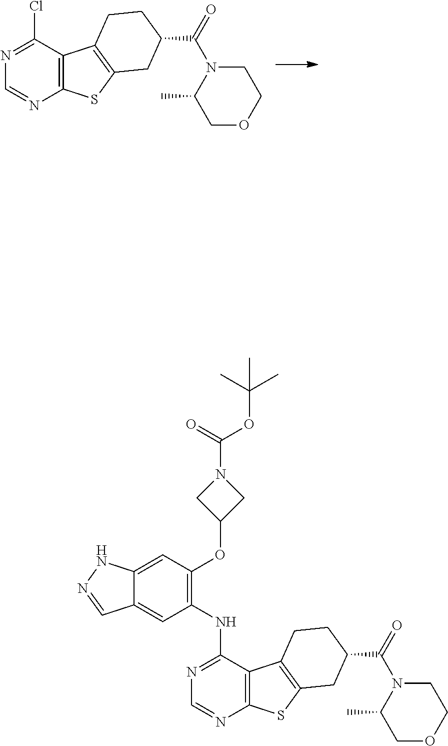 [SMILES: C[C@H]1COCCN1C(=O)[C@H]1CCc2c(sc3ncnc(Cl)c23)C1 ; C[C@H]1COCCN1C(=O)[C@H]1CCc2c(sc3ncnc(Nc4cc5cn[nH]c5cc4OC4CN(C(=O)OC(C)(C)C)C4)c23)C1]

120 mg (341 μmol) [(7S)-4-chloro-5,6,7,8-tetrahydro[1]benzothieno[2,3-d]pyrimidin-7-yl][(3S)-3-methylmorpholin-4-yl]methanone (prepared according to intermediate example 117a) were transformed in analogy to intermediate example 1b using tert-butyl 3-[(5-amino-1H-indazol-6-yl)oxy]azetidine-1-carboxylate (prepared according to intermediate example 265a) to give after working up and purification 27.6 mg (12%) of the title compound.

¹H-NMR (DMSO-d6): δ=1.15+1.29 (3H), 1.38 (9H), 1.85 (1H), 2.09 (1H), 2.83-3.73 (8H), 3.75-3.96 (4H), 4.12 (1H), 4.30-4.49 (3H), 5.25 (1H), 6.87 (1H), 8.02 (1H), 8.27 (1H), 8.51 (1H), 8.98 (1H), 12.85 (1H) ppm.

### Example 268

### [(7S)-4-{[6-(Azetidin-3-yloxy)-1H-indazol-5-yl]amino}-5,6,7,8-tetrahydro[1]benzothieno[2,3-d]pyrimidin-7-yl][(3S)-3-methylmorpholin-4-yl]methanone

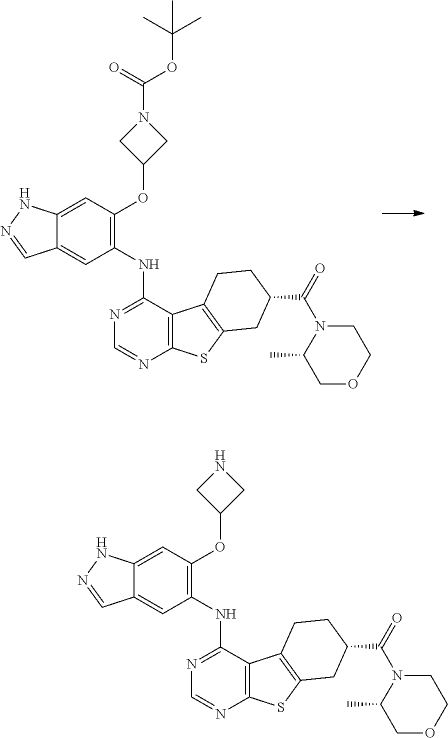 [SMILES: C[C@H]1COCCN1C(=O)[C@H]1CCc2c(sc3ncnc(Nc4cc5cn[nH]c5cc4OC4CN(C(=O)OC(C)(C)C)C4)c23)C1 ; C[C@H]1COCCN1C(=O)[C@H]1CCc2c(sc3ncnc(Nc4cc5cn[nH]c5cc4OC4CNC4)c23)C1]

A mixture comprising 19.7 mg (32 μmol) tert-Butyl 3-[(5-{[(7S)-7-{[(3S)-3-methylmorpholin-4-yl]carbonyl}-5,6,7,8-tetrahydro[1]benzothieno[2,3-d]pyrimidin-4-yl]amino}-1H-indazol-6-yl)oxy]azetidine-1-carboxylate (prepared according to example 267), 1.23 mL dichloromethane and 147 μL trifluoroacetic acid was stirred at 23° C. for one hour. The organic solvents were removed, N,N-diethylethanamine was added, the precipitate collected, washed with water and dried to give 12.7 mg (73%) of the title compound.

¹H-NMR (DMSO-d6): δ=1.16+1.29 (3H), 1.87 (1H), 2.07 (1H), 2.84-3.69 (12H), 3.75-4.48 (5H), 5.20 (1H), 6.80 (1H), 8.00 (1H), 8.29 (1H), 8.51 (1H), 8.98 (1H), 12.77 (1H) ppm.

### Example 269

### (7S)—N,N-Dimethyl-4-({6-[2-(propanoylamino)ethoxy]-1H-indazol-5-yl}amino)-5,6,7,8-tetrahydro[1]benzothieno[2,3-d]pyrimidine-7-carboxamide

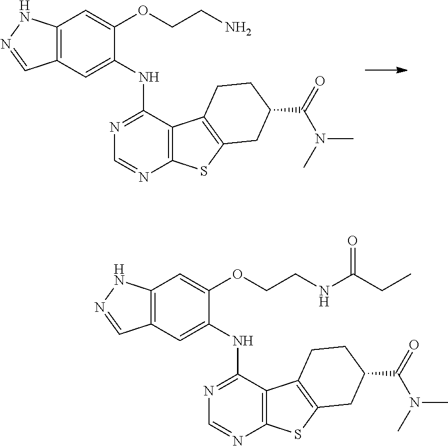 [SMILES: CN(C)C(=O)[C@H]1CCc2c(sc3ncnc(Nc4cc5cn[nH]c5cc4OCCN)c23)C1 ; CCC(=O)NCCOc1cc2[nH]ncc2cc1Nc1ncnc2sc3c(c12)CC[C@H](C(=O)N(C)C)C3]

30 mg (66 μmol) (7S)-4-{[6-(2-aminoethoxy)-1H-indazol-5-yl]amino}-N,N-dimethyl-5,6,7,8-tetrahydro[1]benzothieno[2,3-d]pyrimidine-7-carboxamide (prepared according to example 108) were transformed in analogy to intermediate example 216a using propanoyl chloride to give after working up and purification 5.4 mg (15%) of the title compound.

¹H-NMR (DMSO-d6): δ=0.96 (3H), 1.79 (1H), 2.06 (1H), 2.09 (2H), 2.88 (3H), 2.93 (2H), 3.11 (3H), 3.18 (2H), 3.32 (1H), 3.58 (2H), 4.24 (2H), 7.11 (1H), 8.01 (1H), 8.11 (1H), 8.30 (1H), 8.53 (1H), 9.01 (1H), 12.91 (1H) ppm.

### Example 270

### (7S)-4-({6-[2-(Butanoylamino)ethoxy]-1H-indazol-5-yl}amino)-N,N-dimethyl-5,6,7,8-tetrahydro[1]benzothieno[2,3-d]pyrimidine-7-carboxamide

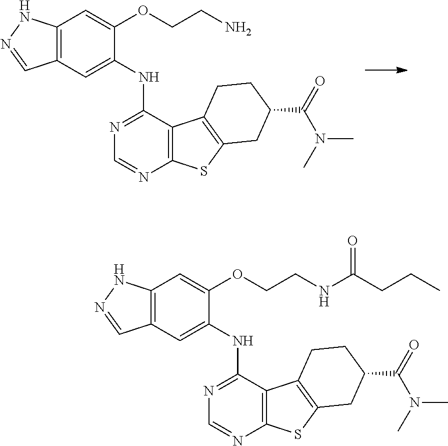 [SMILES: CN(C)C(=O)[C@H]1CCc2c(sc3ncnc(Nc4cc5cn[nH]c5cc4OCCN)c23)C1 ; CCCC(=O)NCCOc1cc2[nH]ncc2cc1Nc1ncnc2sc3c(c12)CC[C@H](C(=O)N(C)C)C3]

30 mg (66 μmol) (7S)-4-{[6-(2-aminoethoxy)-1H-indazol-5-yl]amino}-N,N-dimethyl-5,6,7,8-tetrahydro[1]benzothieno[2,3-d]pyrimidine-7-carboxamide (prepared according to example 108) were transformed in analogy to intermediate example 216a using butanoyl chloride to give after working up and purification 4.9 mg (13%) of the title compound.

¹H-NMR (DMSO-d6): δ=0.80 (3H), 1.48 (2H), 1.79 (1H), 2.05 (2H), 2.08 (1H), 2.88 (3H), 2.94 (2H), 3.11 (3H), 3.17 (2H), 3.25 (1H), 3.54 (1H), 3.63 (1H), 4.24 (2H), 7.11 (1H), 8.02 (1H), 8.08 (1H), 8.30 (1H), 8.53 (1H), 9.00 (1H), 12.86 (1H) ppm.

### Example 271

### (7S)—N,N-Dimethyl-4-[(6-{2-[(3-methylbutanoyl)amino]ethoxy}-1H-indazol-5-yl)amino]-5,6,7,8-tetrahydro[1]benzothieno[2,3-d]pyrimidine-7-carboxamide

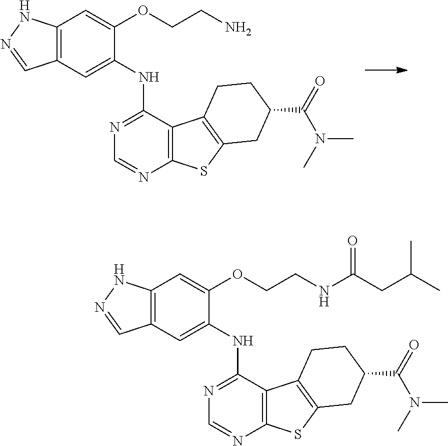 [SMILES: CN(C)C(=O)[C@H]1CCc2c(sc3ncnc(Nc4cc5cn[nH]c5cc4OCCN)c23)C1 ; CC(C)CC(=O)NCCOc1cc2[nH]ncc2cc1Nc1ncnc2sc3c(c12)CC[C@H](C(=O)N(C)C)C3]

30 mg (66 μmol) (7S)-4-{[6-(2-aminoethoxy)-1H-indazol-5-yl]amino}-N,N-dimethyl-5,6,7,8-tetrahydro[1]benzothieno[2,3-d]pyrimidine-7-carboxamide (prepared according to example 108) were transformed in analogy to intermediate example 216a using 3-methylbutanoyl chloride to give after working up and purification 3.8 mg (10%) of the title compound.

¹H-NMR (DMSO-d6): δ=0.81 (6H), 1.80 (1H), 1.88-2.00 (3H), 2.08 (1H), 2.88 (3H), 2.94 (2H), 3.11 (3H), 3.17 (2H), 3.26 (1H), 3.54 (1H), 3.64 (1H), 4.23 (2H), 7.11 (1H), 8.02 (1H), 8.07 (1H), 8.28 (1H), 8.53 (1H), 8.99 (1H), 12.85 (1H) ppm.

### Example 272

### (7S)-4-[(6-{2-[(3,3-Dimethylbutanoyl)amino]ethoxy}-1H-indazol-5-yl)amino]-N,N-dimethyl-5,6,7,8-tetrahydro[1]benzothieno[2,3-d]pyrimidine-7-carboxamide

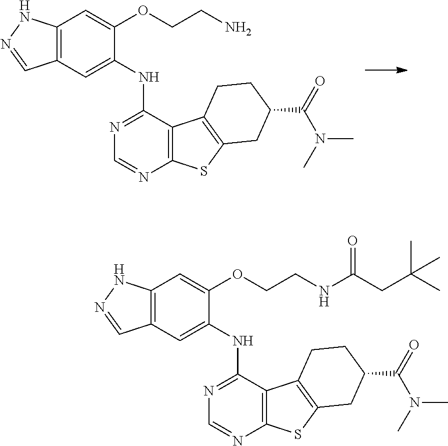 [SMILES: CN(C)C(=O)[C@H]1CCc2c(sc3ncnc(Nc4cc5cn[nH]c5cc4OCCN)c23)C1 ; CN(C)C(=O)[C@H]1CCc2c(sc3ncnc(Nc4cc5cn[nH]c5cc4OCCNC(=O)CC(C)(C)C)c23)C1]

30 mg (66 μmol) (7S)-4-{[6-(2-aminoethoxy)-1H-indazol-5-yl]amino}-N,N-dimethyl-5,6,7,8-tetrahydro[1]benzothieno[2,3-d]pyrimidine-7-carboxamide (prepared according to example 108) were transformed in analogy to intermediate example 216a using 3,3-dimethylbutanoyl chloride to give after working up and purification 6.2 mg (16%) of the title compound.

¹H-NMR (DMSO-d6): δ=0.89 (9H), 1.81 (1H), 1.96 (2H), 2.10 (1H), 2.88 (3H), 2.94 (2H), 3.11 (3H), 3.17 (2H), 3.27 (1H), 3.53 (1H), 3.64 (1H), 4.22 (2H), 7.10 (1H), 7.99 (1H), 8.02 (1H), 8.28 (1H), 8.52 (1H), 8.98 (1H), 12.85 (1H) ppm.

### Example 273

### (7S)-4-[(6-{2-[(Cyclopentylacetyl)amino]ethoxy}-1H-indazol-5-yl)amino]-N,N-dimethyl-5,6,7,8-tetrahydro[1]benzothieno[2,3-d]pyrimidine-7-carboxamide

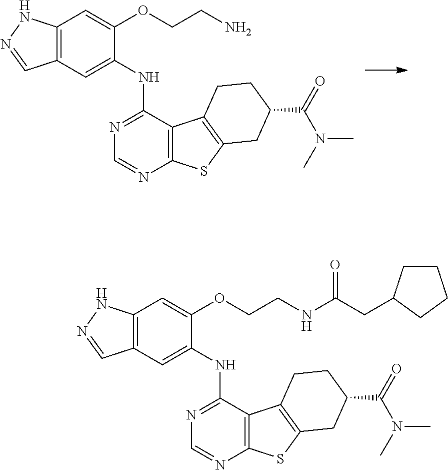 [SMILES: CN(C)C(=O)[C@H]1CCc2c(sc3ncnc(Nc4cc5cn[nH]c5cc4OCCN)c23)C1 ; CN(C)C(=O)[C@H]1CCc2c(sc3ncnc(Nc4cc5cn[nH]c5cc4OCCNC(=O)CC4CCCC4)c23)C1]

30 mg (66 μmol) (7S)-4-{[6-(2-aminoethoxy)-1H-indazol-5-yl]amino}-N,N-dimethyl-5,6,7,8-tetrahydro[1]benzothieno[2,3-d]pyrimidine-7-carboxamide (prepared according to example 108) were transformed in analogy to intermediate example 216a using cyclopentylacetyl chloride to give after working up and purification 5.1 mg (13%) of the title compound.

¹H-NMR (DMSO-d6): δ=1.04 (2H), 1.37 (2H), 1.47 (2H), 1.61 (2H), 1.80 (1H), 2.06 (4H), 2.88 (3H), 2.93 (2H), 3.04-3.29 (3H), 3.11 (3H), 3.53 (1H), 3.65 (1H), 4.23 (2H), 7.10 (1H), 8.01 (1H), 8.04 (1H), 8.27 (1H), 8.52 (1H), 8.98 (1H), 12.86 (1H) ppm.

### Example 274

### (7S)-4-[(6-{2-[(Cyclohexylacetyl)amino]ethoxy}-1H-indazol-5-yl)amino]-N,N-dimethyl-5,6,7,8-tetrahydro[1]benzothieno[2,3-d]pyrimidine-7-carboxamide

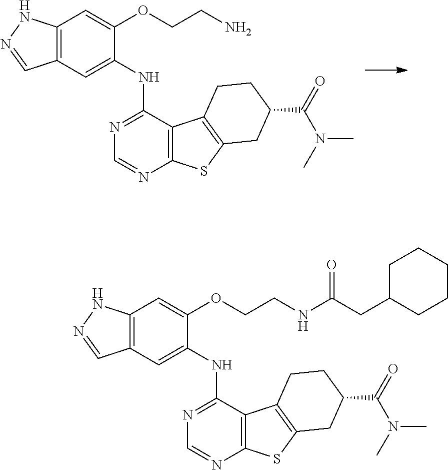 [SMILES: CN(C)C(=O)[C@H]1CCc2c(sc3ncnc(Nc4cc5cn[nH]c5cc4OCCN)c23)C1 ; CN(C)C(=O)[C@H]1CCc2c(sc3ncnc(Nc4cc5cn[nH]c5cc4OCCNC(=O)CC4CCCCC4)c23)C1]

30 mg (66 μmol) (7S)-4-{[6-(2-aminoethoxy)-1H-indazol-5-yl]amino}-N,N-dimethyl-5,6,7,8-tetrahydro[1]benzothieno[2,3-d]pyrimidine-7-carboxamide (prepared according to example 108) were transformed in analogy to intermediate example 216a using cyclohexylacetyl chloride to give after working up and purification 4.4 mg (11%) of the title compound.

¹H-NMR (DMSO-d6): δ=0.82 (2H), 0.96-1.14 (3H), 1.45-1.66 (6H), 1.80 (1H), 1.95 (2H), 2.10 (1H), 2.89 (3H), 2.94 (2H), 3.10-3.33 (3H), 3.12 (3H), 3.52 (1H), 3.66 (1H), 4.24 (2H), 7.10 (1H), 8.02 (1H), 8.04 (1H), 8.28 (1H), 8.53 (1H), 9.00 (1H), 12.86 (1H) ppm.

### Example 275

### (7S)-4-[(6-{2-[(2,2-Dimethylpropyl)amino]ethoxy}-1H-indazol-5-yl)amino]-N,N-dimethyl-5,6,7,8-tetrahydro[1]benzothieno[2,3-d]pyrimidine-7-carboxamide

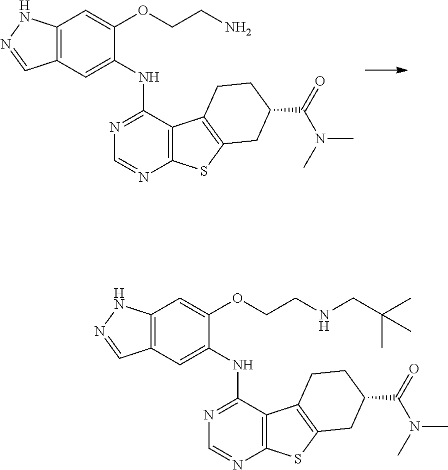 [SMILES: CN(C)C(=O)[C@H]1CCc2c(sc3ncnc(Nc4cc5cn[nH]c5cc4OCCN)c23)C1 ; CN(C)C(=O)[C@H]1CCc2c(sc3ncnc(Nc4cc5cn[nH]c5cc4OCCNCC(C)(C)C)c23)C1]

To a mixture comprising 100 mg (221 μmol) (7S)-4-{[6-(2-aminoethoxy)-1H-indazol-5-yl]amino}-N,N-dimethyl-5,6,7,8-tetrahydro[1]benzothieno[2,3-d]pyrimidine-7-carboxamide (prepared according to example 108), 5 mL methanol, 200 μL acetic acid, 22.3 mg sodium (cyano-kappaC)(trihydrido)borate (1−) were added the solution of 24 μL pivalaldehyde in 2 mL methanol at 0° C. and the was stirred at 23° C. overnight. The same amount of sodium (cyano-kappaC)(trihydrido)borate (1−) and pivalaldehyde were added as described supra and stirring was continued. Dichloromethane and sodium carbonate solution was added and the mixture extracted with dichloromethane. The combined organic layers were washed with brine and dried over sodium sulphate. After filtration and removal of the solvents, the residue was purified by crystallization from N,N-dimethylformamide to give 5.3 mg (4%) of the title compound.

¹H-NMR (DMSO-d6): δ=0.83 (9H), 1.85 (1H), 2.14 (1H), 2.33 (2H), 2.89 (3H), 2.95 (2H), 3.02 (2H), 3.11 (3H), 3.14-3.38 (3H), 4.24 (2H), 7.11 (1H), 8.01 (1H), 8.35 (1H), 8.51 (1H), 8.94 (1H), 12.84 (1H) ppm.

### Example 276

### (7S)-4-({6-[3-(3-Fluoroazetidin-1-yl)propoxy]-1H-indazol-5-yl}amino)-N,N-dimethyl-5,6,7,8-tetrahydro[1]benzothieno[2,3-d]pyrimidine-7-carboxamide

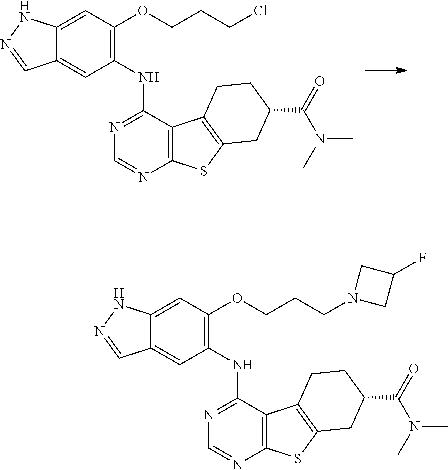 [SMILES: CN(C)C(=O)[C@H]1CCc2c(sc3ncnc(Nc4cc5cn[nH]c5cc4OCCCCl)c23)C1 ; CN(C)C(=O)[C@H]1CCc2c(sc3ncnc(Nc4cc5cn[nH]c5cc4OCCCN4CC(F)C4)c23)C1]

A mixture comprising 93 mg (192 μmol) (7S)-4-{[6-(3-chloropropoxy)-1H-indazol-5-yl]amino}-N,N-dimethyl-5,6,7,8-tetrahydro[1]benzothieno[2,3-d]pyrimidine-7-carboxamide (prepared according to intermediate example 276a), 4 mL tetrahydrofuran, 6.4 mg potassium iodide, 159 mg potassium carbonate and 42.8 mg 3-fluoroazetidine was stirred at 80° C. for 2.5 days. 6.4 mg potassium iodide, 53 mg potassium carbonate and 42.8 mg 3-fluoroazetidine were added and stirring continued for 1 day. The mixture was poured into ammonium chloride solution, the precipitate filtered off and purified by chromatography to give 14.0 mg (14%) of the title compound.

¹H-NMR (DMSO-d6): δ=1.78-2.04 (3H), 2.13 (1H), 2.90 (3H), 2.95 (2H), 3.06-3.98 (9H), 3.12 (3H), 4.20 (2H), 5.07-5.36 (1H), 7.09 (1H), 8.01 (1H), 8.27 (1H), 8.51 (1H), 8.91 (1H), 12.86 (1H) ppm.

### Example 276a

### (7S)-4-{[6-(3-Chloropropoxy)-1H-indazol-5-yl]amino}-N,N-dimethyl-5,6,7,8-tetrahydro[1]benzothieno[2,3-d]pyrimidine-7-carboxamide

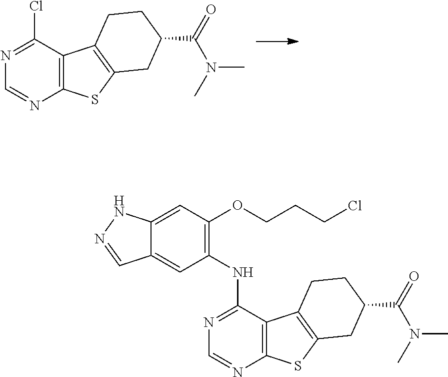 [SMILES: CN(C)C(=O)[C@H]1CCc2c(sc3ncnc(Cl)c23)C1 ; CN(C)C(=O)[C@H]1CCc2c(sc3ncnc(Nc4cc5cn[nH]c5cc4OCCCCl)c23)C1]

550 mg (1.86 mmol) (7S)-4-chloro-N,N-dimethyl-5,6,7,8-tetrahydro[1]benzothieno[2,3-d]pyrimidine-7-carboxamide (prepared according to intermediate example 94a) were transformed in analogy to intermediate example 1b using 6-(3-chloropropoxy)-1H-indazol-5-amine (prepared according to intermediate example 110b) to give after working up and purification 535 mg (59%) of the title compound.

### Example 277

### (7S)—N,N-Dimethyl-4-({6-[3-(pyrrolidin-1-yl)propoxy]-1H-indazol-5-yl}amino)-5,6,7,8-tetrahydro[1]benzothieno[2,3-d]pyrimidine-7-carboxamide

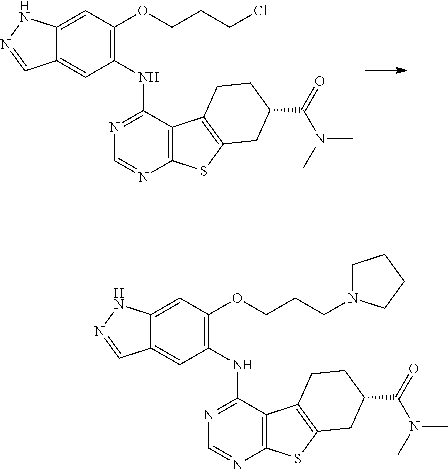 [SMILES: CN(C)C(=O)[C@H]1CCc2c(sc3ncnc(Nc4cc5cn[nH]c5cc4OCCCCl)c23)C1 ; CN(C)C(=O)[C@H]1CCc2c(sc3ncnc(Nc4cc5cn[nH]c5cc4OCCCN4CCCC4)c23)C1]

70 mg (144 μmol) (7S)-4-{[6-(3-chloropropoxy)-1H-indazol-5-yl]amino}-N,N-dimethyl-5,6,7,8-tetrahydro[1]benzothieno[2,3-d]pyrimidine-7-carboxamide (prepared according to intermediate example 276a) were transformed in analogy to example 276 using pyrrolidine to give after working up and purification 4.0 mg (5%) of the title compound.

¹H-NMR (DMSO-d6): δ=1.69 (4H), 1.84 (1H), 2.04 (2H), 2.16 (1H), 2.54 (2H), 2.62 (2H), 2.89 (3H), 2.95 (2H), 3.11 (3H), 3.15-3.40 (4H), 4.22 (2H), 7.09 (1H), 8.01 (1H), 8.16 (1H), 8.30 (1H), 8.51 (1H), 8.93 (1H), 12.84 (1H) ppm.

### Example 278

### (7S)—N,N-Dimethyl-4-({6-[3-(piperidin-1-yl)propoxy]-1H-indazol-5-yl}amino)-5,6,7,8-tetrahydro[1]benzothieno[2,3-d]pyrimidine-7-carboxamide

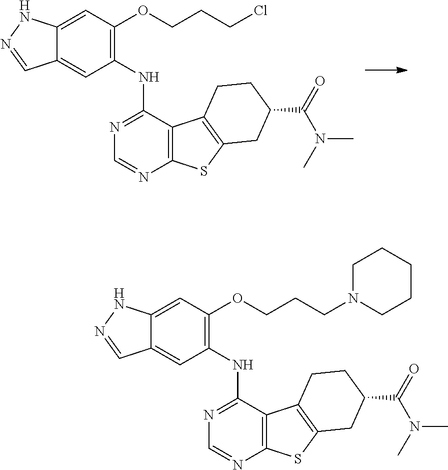 [SMILES: CN(C)C(=O)[C@H]1CCc2c(sc3ncnc(Nc4cc5cn[nH]c5cc4OCCCCl)c23)C1 ; CN(C)C(=O)[C@H]1CCc2c(sc3ncnc(Nc4cc5cn[nH]c5cc4OCCCN4CCCCC4)c23)C1]

71 mg (146 μmol) (7S)-4-{[6-(3-chloropropoxy)-1H-indazol-5-yl]amino}-N,N-dimethyl-5,6,7,8-tetrahydro[1]benzothieno[2,3-d]pyrimidine-7-carboxamide (prepared according to intermediate example 276a) were transformed in analogy to example 276 using piperidine to give after working up and purification 9.0 mg (11%) of the title compound.

¹H-NMR (DMSO-d6): δ=1.38 (2H), 1.48 (4H), 1.84 (1H), 2.01 (2H), 2.17 (1H), 2.26-2.37 (3H), 2.42 (2H), 2.89 (3H), 2.95 (2H), 3.12 (3H), 3.14-3.30 (3H), 4.18 (2H), 7.08 (1H), 8.01 (1H), 8.18 (1H), 8.31 (1H), 8.51 (1H), 8.94 (1H), 12.83 (1H) ppm.

### Example 279

### [(7S)-4-{[6-(2, 2-Dimethylpropoxy)-1H-indazol-5-yl]amino}-5,6,7,8-tetrahydro[1]benzothieno[2,3-d]pyrimidin-7-yl](morpholin-4-yl)methanone

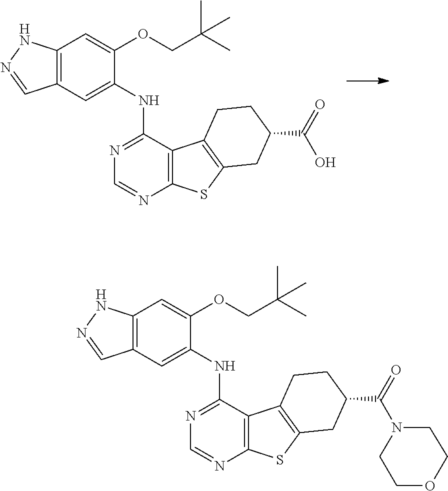 [SMILES: CC(C)(C)COc1cc2[nH]ncc2cc1Nc1ncnc2sc3c(c12)CC[C@H](C(=O)O)C3 ; CC(C)(C)COc1cc2[nH]ncc2cc1Nc1ncnc2sc3c(c12)CC[C@H](C(=O)N1CCOCC1)C3]

100 mg (221 μmol) (7S)-4-{[6-(2,2-dimethylpropoxy)-1H-indazol-5-yl]amino}-5,6,7,8-tetrahydro[1]benzothieno[2,3-d]pyrimidine-7-carboxylic acid (prepared according to intermediate example 279a) were transformed in analogy to example 1 using morpholine to give after working up and purification 3.1 mg (3%) of the title compound.

¹H-NMR (DMSO-d6): δ=0.99 (9H), 1.85 (1H), 2.03 (1H), 2.95 (2H), 3.22 (2H), 3.32 (1H), 3.44-3.66 (8H), 3.80 (2H), 7.07 (1H), 7.98 (1H), 8.08 (1H), 8.42 (1H), 8.69 (1H), 12.79 (1H) ppm.

### Example 279a

### (7S)-4-{[6-(2,2-Dimethylpropoxy)-1H-indazol-5-yl]amino}-5,6,7,8-tetrahydro[1]benzothieno[2,3-d]pyrimidine-7-carboxylic acid

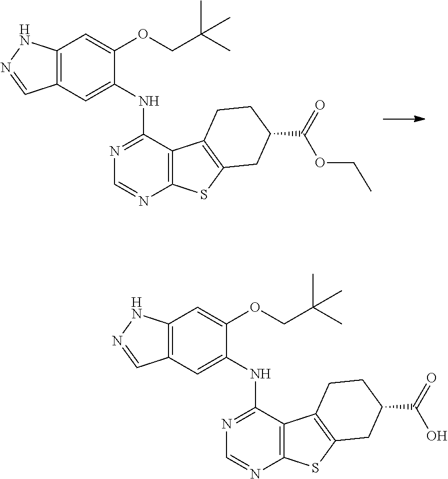 [SMILES: CCOC(=O)[C@H]1CCc2c(sc3ncnc(Nc4cc5cn[nH]c5cc4OCC(C)(C)C)c23)C1 ; CC(C)(C)COc1cc2[nH]ncc2cc1Nc1ncnc2sc3c(c12)CC[C@H](C(=O)O)C3]

780 mg (1.63 mmol) ethyl (7S)-4-{[6-(2,2-dimethylpropoxy)-1H-indazol-5-yl]amino}-5,6,7,8-tetrahydro[1]benzothieno[2,3-d]pyrimidine-7-carboxylate (prepared according to intermediate example 279b) were transformed in analogy to intermediate example 1a to give after working up and purification 641 mg (87%) of the title compound.

### Example 279a

### Ethyl (7S)-4-{[6-(2,2-dimethylpropoxy)-1H-indazol-5-yl]amino}-5,6,7,8-tetrahydro[1]benzothieno[2,3-d]pyrimidine-7-carboxylate

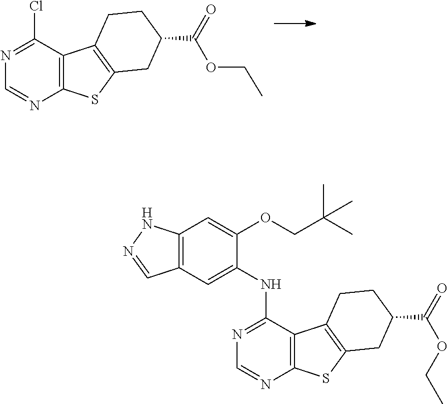 [SMILES: CCOC(=O)[C@H]1CCc2c(sc3ncnc(Cl)c23)C1 ; CCOC(=O)[C@H]1CCc2c(sc3ncnc(Nc4cc5cn[nH]c5cc4OCC(C)(C)C)c23)C1]

712 mg (2.40 mmol) ethyl (7S)-4-chloro-5,6,7,8-tetrahydro[1]benzothieno[2,3-d]pyrimidine-7-carboxylate (prepared according to intermediate example 1c) were transformed in analogy to intermediate example 1b using 6-(2,2-dimethylpropoxy)-1H-indazol-5-amine (prepared according to intermediate example 176a) to give after working up and purification 718 mg (62%) of the title compound.

### Example 280

### (7S)—N,N-Dimethyl-4-({6-[(3S)-tetrahydrofuran-3-yloxy]-1H-indazol-5-yl}amino)-5,6,7,8-tetrahydro[1]benzothieno[2,3-d]pyrimidine-7-carboxamide

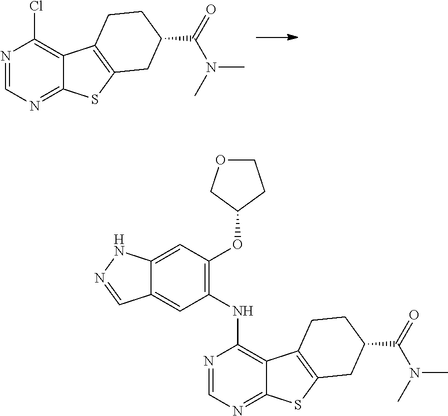 [SMILES: CN(C)C(=O)[C@H]1CCc2c(sc3ncnc(Cl)c23)C1 ; CN(C)C(=O)[C@H]1CCc2c(sc3ncnc(Nc4cc5cn[nH]c5cc4O[C@H]4CCOC4)c23)C1]

103 mg (347 μmol) (7S)-4-chloro-N,N-dimethyl-5,6,7,8-tetrahydro[1]benzothieno[2,3-d]pyrimidine-7-carboxamide (prepared according to intermediate example 94a) were transformed in analogy to intermediate example 1b using 6-[(3S)-tetrahydrofuran-3-yloxy]-1H-indazol-5-amine (prepared according to intermediate example 280a) to give after working up and purification 64.2 mg (38%) of the title compound.

¹H-NMR (DMSO-d6): δ=1.84 (1H), 2.11 (2H), 2.32 (1H), 2.89 (3H), 2.94 (2H), 3.11 (3H), 3.17-3.30 (3H), 3.81-4.04 (4H), 5.34 (1H), 7.11 (1H), 8.02 (1H), 8.32 (1H), 8.54 (1H), 9.06 (1H), 12.84 (1H) ppm.

### Example 280a

### 6-[(3S)-Tetrahydrofuran-3-yloxy]-1H-indazol-5-amine

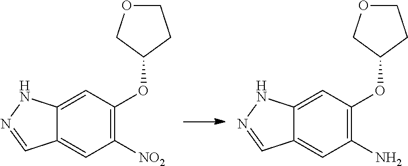 [SMILES: O=[N+]([O-])c1cc2cn[nH]c2cc1O[C@H]1CCOC1 ; Nc1cc2cn[nH]c2cc1O[C@H]1CCOC1]

960 mg (3.85 mmol) 5-nitro-6-[(3S)-tetrahydrofuran-3-yloxyl H-indazole (prepared according to intermediate example 280b) were transformed in analogy to intermediate example 94b to give after working up and purification 542 mg (64%) of the title compound.

### Example 280b

### 5-Nitro-6-[(3S)-tetrahydrofuran-3-yloxy]-1H-indazole

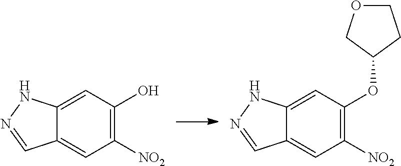 [SMILES: O=[N+]([O-])c1cc2cn[nH]c2cc1O ; O=[N+]([O-])c1cc2cn[nH]c2cc1O[C@H]1CCOC1]

1.00 g (5.58 mmol) 5-nitro-1H-indazol-6-ol (prepared according to intermediate example 94d) were transformed in analogy to intermediate example 94c using (3S)-tetrahydrofuran-3-ol to give after working up and purification 530 mg (38%) of the title compound.

### Example 281

### (7S)-4-[(6-Methoxy-1H-indazol-5-yl)amino]-N-[(2R)-2-methoxypropyl]-N-methyl-5,6,7,8-tetrahydro[1]benzothieno[2,3-d]pyrimidine-7-carboxamide

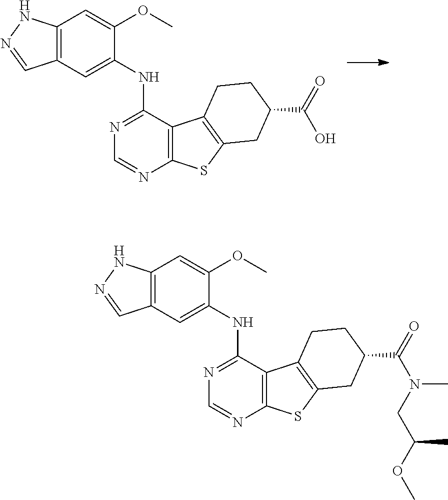 [SMILES: COc1cc2[nH]ncc2cc1Nc1ncnc2sc3c(c12)CC[C@H](C(=O)O)C3 ; COc1cc2[nH]ncc2cc1Nc1ncnc2sc3c(c12)CC[C@H](C(=O)N(C)C[C@@H](C)OC)C3]

113 mg (286 μmol) (7S)-4-[(6-methoxy-1H-indazol-5-yl)amino]-5,6,7,8-tetrahydro[1]benzothieno[2,3-d]pyrimidine-7-carboxylic acid (prepared according to intermediate example 20a) were transformed in analogy to example 1 using (2R)-2-methoxy-N-methylpropan-1-amine (prepared according to intermediate example 281a) to give after working up and purification 91.8 mg (63%) of the title compound.

¹H-NMR (DMSO-d6): δ=1.05+1.09 (3H), 1.85 (1H), 2.10+2.17 (1H), 2.82-3.05 (2H), 2.90+3.16 (3H), 3.12-3.37 (7H), 3.43+3.63 (1H), 3.54 (1H), 4.00 (3H), 7.10 (1H), 8.01 (1H), 8.23 (1H), 8.48 (1H), 8.79+8.81 (1H), 12.86 (1H) ppm.

### Example 281a

### (2R)-2-Methoxy-N-methylpropan-1-amine

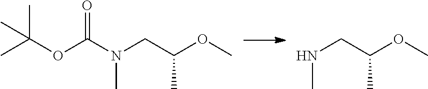

A mixture comprising 1.14 g (5.61 mmol) tert-butyl [(2R)-2-methoxypropyl]methylcarbamate (prepared according to intermediate example 281b) and 14 mL hydrochloric acid (4M in dioxane) was heated at 50° C. for 18 hours. The solvents were removed, the residue digested with diethyl ether and the precipitate was dried to give 423 mg (51%) of the title compound as salt with hydrochloric acid.

### Example 281b

### tert-Butyl [(2R)-2-methoxypropyl]methylcarbamate

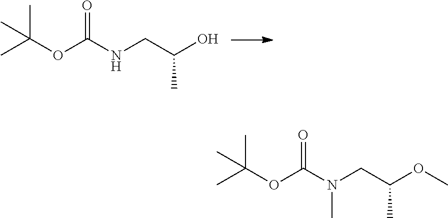

From 1.83 g sodium hydride (60%) the mineral oil was removed and 60 mL tetrahydrofuran were added followed by 2.84 mL iodomethane and 2.00 g (11.4 mmol) tert-butyl [(2R)-2-hydroxypropyl]carbamate (CAS-No: 119768-44-4) solved in 10 mL tetrahydrofuran. The mixture was stirred at 23° C. for 4 hours, methanol was added and the solvents were removed. Dichloromethane was added and the suspension was purified by chromatography to give 946 mg (41%) of the title compound.

### Example 282

### (7S)-4-[(6-Methoxy-1H-indazol-5-yl)amino]-N-[(2S)-2-methoxypropyl]-N-methyl-5,6,7,8-tetrahydro[1]benzothieno[2,3-d]pyrimidine-7-carboxamide

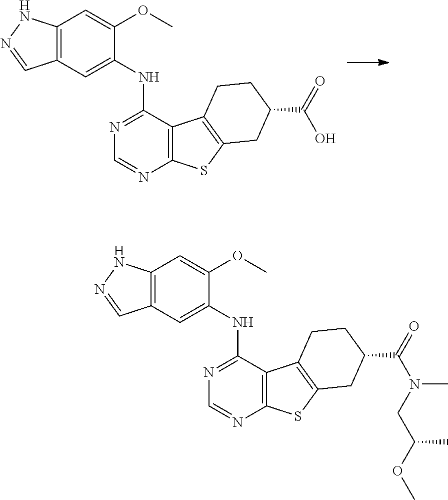 [SMILES: COc1cc2[nH]ncc2cc1Nc1ncnc2sc3c(c12)CC[C@H](C(=O)O)C3 ; COc1cc2[nH]ncc2cc1Nc1ncnc2sc3c(c12)CC[C@H](C(=O)N(C)C[C@H](C)OC)C3]

113 mg (286 μmol) (7S)-4-[(6-methoxy-1H-indazol-5-yl)amino]-5,6,7,8-tetrahydro[1]benzothieno[2,3-d]pyrimidine-7-carboxylic acid (prepared according to intermediate example 20a) were transformed in analogy to example 1 using (2S)-2-methoxy-N-methylpropan-1-amine (prepared according to intermediate example 282a) to give after working up and purification 87.7 mg (61%) of the title compound.

¹H-NMR (DMSO-d6): δ=1.05+1.09 (3H), 1.87 (1H), 2.15 (1H), 2.91+3.16 (3H), 2.94 (2H), 3.13-3.59 (9H), 3.98+4.00 (3H), 7.10 (1H), 8.01 (1H), 8.23 (1H), 8.46+8.48 (1H), 8.76+8.81 (1H), 12.86 (1H) ppm.

### Example 282a

### (2S)-2-Methoxy-N-methylpropan-1-amine

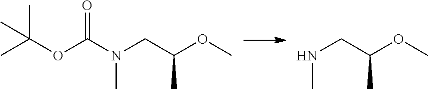

797 mg (3.92 mmol) tert-butyl [(2S)-2-methoxypropyl]methylcarbamate (prepared according to intermediate example 282b) were transformed in analogy to intermediate example 281a to give after working up and purification 404 mg (74%) of the title compound.

### Example 282b

### tert-Butyl [(2S)-2-methoxypropyl]methylcarbamate

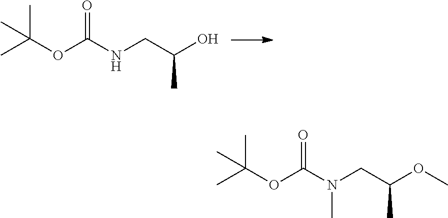

2.00 g (11.4 mmol) tert-butyl [(2S)-2-hydroxypropyl]carbamate (CAS-No: 167938-56-9) were transformed in analogy to intermediate example 281b to give after working up and purification 797 mg (34%) of the title compound.

### Example 283

### (7S)-4-[(6-Methoxy-1H-indazol-5-yl)amino]-N-[(25)-1-methoxypropan-2-yl]-N-methyl-5,6,7,8-tetrahydro[1]benzothieno[2,3-d]pyrimidine-7-carboxamide

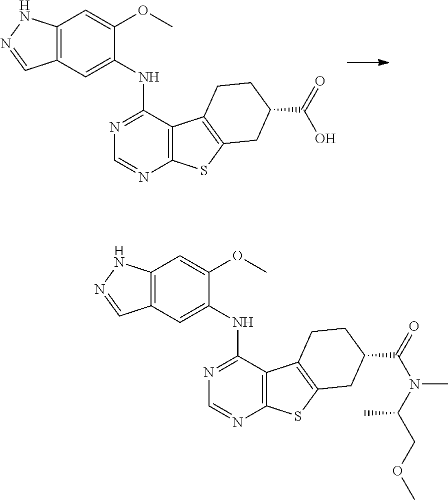 [SMILES: COc1cc2[nH]ncc2cc1Nc1ncnc2sc3c(c12)CC[C@H](C(=O)O)C3 ; COC[C@H](C)N(C)C(=O)[C@H]1CCc2c(sc3ncnc(Nc4cc5cn[nH]c5cc4OC)c23)C1]

113 mg (286 μmol) (7S)-4-[(6-methoxy-1H-indazol-5-yl)amino]-5,6,7,8-tetrahydro[1]benzothieno[2,3-d]pyrimidine-7-carboxylic acid (prepared according to intermediate example 20a) were transformed in analogy to example 1 using (2S)-1-methoxy-N-methylpropan-2-amine (prepared according to intermediate example 281a) to give after working up and purification 82.4 mg (57%) of the title compound.

¹H-NMR (DMSO-d6): δ=1.04+1.12 (3H), 1.85 (1H), 2.16 (1H), 2.72+2.95 (3H), 2.85-3.04 (2H), 3.11-3.48 (5H), 3.25+3.32 (3H), 3.97+4.00 (3H), 4.33+4.78 (1H), 7.10 (1H), 8.01 (1H), 8.22+8.24 (1H), 8.48 (1H), 8.75+8.80 (1H), 12.87 (1H) ppm.

### Example 283a

### (2S)-1-Methoxy-N-methylpropan-2-amine

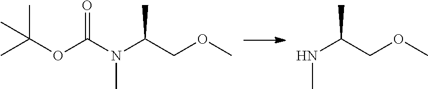

1.00 g (4.92 mmol) tert-butyl [(2S)-1-methoxypropan-2-yl]methylcarbamate (prepared according to intermediate example 283b) were transformed in analogy to intermediate example 281a to give after working up and purification 446 mg (62%) of the title compound.

### Example 283b

### tert-Butyl [(2S)-1-methoxypropan-2-yl]methylcarbamate

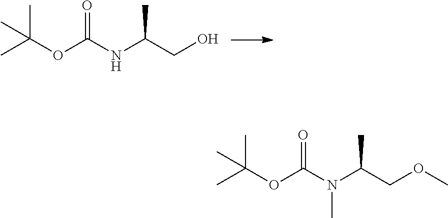

2.00 g (11.4 mmol) tert-butyl [(2S)-1-hydroxypropan-2-yl]carbamate (CAS-No: 79069-13-9) were transformed in analogy to intermediate example 281b to give after working up and purification 880 mg (38%) of the title compound.

### Example 284

### (7S)-4-[(6-Methoxy-1H-indazol-5-yl)amino]-N-[(2R)-1-methoxypropan-2-yl]-N-methyl-5,6,7,8-tetrahydro[1]benzothieno[2,3-d]pyrimidine-7-carboxamide

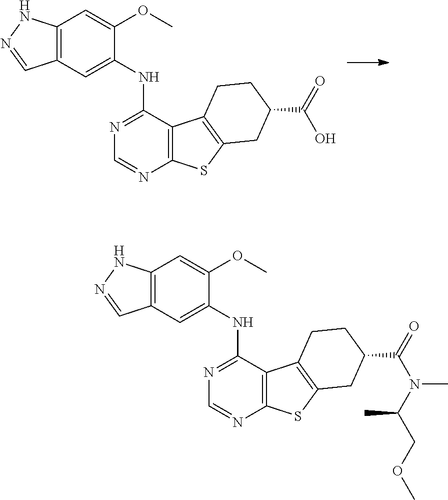 [SMILES: COc1cc2[nH]ncc2cc1Nc1ncnc2sc3c(c12)CC[C@H](C(=O)O)C3 ; COC[C@@H](C)N(C)C(=O)[C@H]1CCc2c(sc3ncnc(Nc4cc5cn[nH]c5cc4OC)c23)C1]

85 mg (215 μmol) (7S)-4-[(6-methoxy-1H-indazol-5-yl)amino]-5,6,7,8-tetrahydro[1]benzothieno[2,3-d]pyrimidine-7-carboxylic acid (prepared according to intermediate example 20a) were transformed in analogy to example 1 using (2R)-1-methoxy-N-methylpropan-2-amine (prepared according to intermediate example 284a) to give after working up and purification 19.9 mg (18%) of the title compound.

¹H-NMR (DMSO-d6): δ=1.03+1.16 (3H), 1.87 (1H), 2.13 (1H), 2.73+2.95 (3H), 2.85-3.07 (2H), 3.13-3.45 (5H), 3.26+3.27 (3H), 4.00+4.01 (3H), 4.33+4.80 (1H), 7.11 (1H), 8.01 (1H), 8.23+8.24 (1H), 8.47+8.48 (1H), 8.78+8.81 (1H), 12.87 (1H) ppm.

### Example 284a

### (2R)-1-Methoxy-N-methylpropan-2-amine

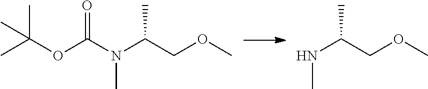

300 mg (1.48 mmol) tert-butyl [(2R)-1-methoxypropan-2-yl]methylcarbamate (prepared according to intermediate example 284b) were transformed in analogy to intermediate example 281a to give after working up and purification 156 mg (72%) of the title compound.

### Example 284b

### tert-Butyl [(2R)-1-methoxypropan-2-yl]methylcarbamate

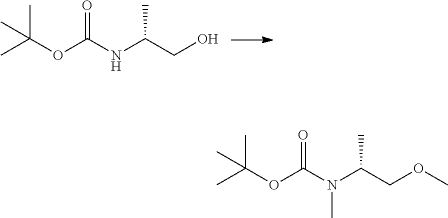

2.00 g (11.4 mmol) tert-butyl [(2R)-1-hydroxypropan-2-yl]carbamate (CAS-No: 106391-86-0) were transformed in analogy to intermediate example 281b to give after working up and purification 1.78 g (76%) of the title compound.

### Example 285

### [3-(dimethylamino)azetidin-1-yl][(7S)-4-{[6-(methylsulfanyl)-1H-indazol-5-yl]amino}-5,6,7,8-tetrahydro[1]benzothieno[2,3-d]pyrimidin-7-yl]methanone

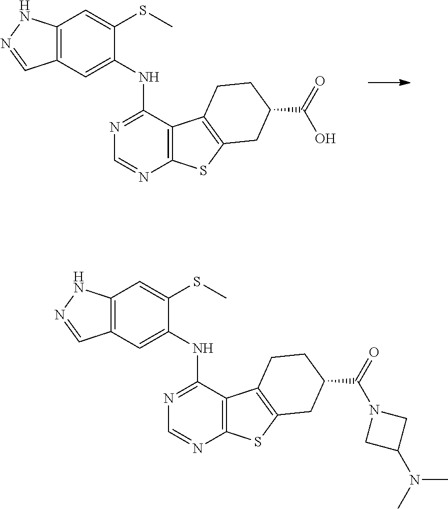 [SMILES: CSc1cc2[nH]ncc2cc1Nc1ncnc2sc3c(c12)CC[C@H](C(=O)O)C3 ; CSc1cc2[nH]ncc2cc1Nc1ncnc2sc3c(c12)CC[C@H](C(=O)N1CC(N(C)C)C1)C3]

75 mg (182 μmol) (7S)-4-{[6-(methylsulfanyl)-1H-indazol-5-yl]amino}-5,6,7,8-tetrahydro[1]benzothieno[2,3-d]pyrimidine-7-carboxylic acid (prepared according to intermediate example 246a) were transformed in analogy to example 1 using N,N-dimethylazetidin-3-amine to give after working up and purification 52.2 mg (55%) of the title compound.

¹H-NMR (DMSO-d6): δ=1.78 (1H), 2.06 (1H), 2.08 (6H), 2.43 (3H), 2.79 (1H), 2.89 (2H), 3.05 (1H), 3.13 (1H), 3.29 (1H), 3.66 (1H), 3.88 (1H), 4.03 (1H), 4.24 (1H), 7.37 (1H), 7.89 (1H), 8.03 (1H), 8.13 (1H), 8.20 (1H), 12.99 (1H) ppm.

### Reference Examples from WO2013/174744(A1)

### Reference Example 4 of WO2013/174744(A1)

### 4-(1H-Indazol-5-ylamino)-N-[3-(methylsulfonyl)propyl]-5,6,7,8-tetrahydro[1]benzothieno[2,3-d]pyrimidine-7-carboxamide

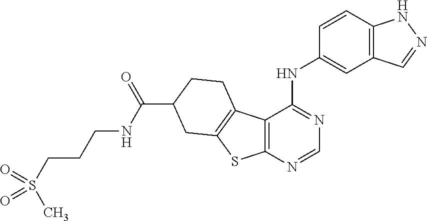 [SMILES: CS(=O)(=O)CCCNC(=O)C1CCc2c(sc3ncnc(Nc4ccc5[nH]ncc5c4)c23)C1]

To a mixture of 4-(1H-indazol-5-ylamino)-5,6,7,8-tetrahydro[1]benzothieno[2,3-d]pyrimidine-7-carboxylic acid (300 mg) and 3-(methylsulfonyl)propyl-1-amine hydrochloride (137 mg) in N,N-dimethylformamide (20 mL) was added N,N-diisopropylethylamine (170 mg), followed by COMU ((1-Cyano-2-ethoxy-2-oxoethylidenaminooxy)dimethylamino-morpholino-carbenium hexafluorophosphate; 422 mg), and the mixture was stirred overnight at room temperature. The mixture was partitioned between water and dichloromethane, and the organic layer was dried over magnesium sulfate and evaporated. To remove undesired impurities, the residue was partitioned between 1 N aqueous hydrochloric acid and dichloromethane, and the aqueous layer was then neutralized by addition of aqueous sodium bicarbonate, followed by dichloromethane, whereupon the target compound precipitated and was isolated by filtration (60 mg).

¹H-NMR (400 MHz, DMSO-d6): δ [ppm]=1.80-1.92 (m, 3H), 2.05-2.16 (m, 1H), 2.60-2.72 (m, 1H), 2.91-3.01 (m, 5H), 3.07-3.29 (m, 6H), 7.45-7.56 (m, 2H), 7.99 (s, 1H), 8.05 (s, 1H), 8.11 (t, 1H), 8.20 (s, 1H), 8.31 (s, 1H), 13.01 (s, 1H).

MS (ESIpos) m/z=485 [M+H]⁺.

### Reference Example 5 of WO2013/174744(A1)

### 4-(1H-Indazol-5-ylamino)-N-phenyl-5,6,7,8-tetrahydro[1]benzothieno[2,3-d]pyrimidine-7-carboxamide

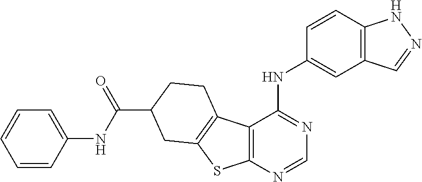 [SMILES: O=C(Nc1ccccc1)C1CCc2c(sc3ncnc(Nc4ccc5[nH]ncc5c4)c23)C1]

To a mixture of 4-(1H-indazol-5-ylamino)-5,6,7,8-tetrahydro[1]benzothieno[2,3-d]pyrimidine-7-carboxylic acid (400 mg) and aniline (399 μL) in N,N-dimethylformamide (12 mL) was added N,N-diisopropylethylamine (915 μL), followed by T3P (propylphosphinic anhydride; 3.13 mL of a 50% solution in ethyl acetate), and the mixture was stirred for 4 h at 60° C. To drive the reaction to completion, aniline (199 μL) was added, followed by N,N-diisopropylamine (458 μL), and T3P (0.78 mL of a 50% solution in ethyl acetate), and the mixture was stirred for another 4 h at 40° C. The mixture was concentrated in vacuo and the residue was purified by preparative HPLC (Method P1) to give 255 mg of the target compound.

¹H-NMR (300 MHz, DMSO-d₆): δ [ppm]=1.85-1.98 (m, 1H), 2.16-2.30 (m, 1H), 2.84-3.42 (m, 5H, partly overlapped with water signal), 7.05 (t, 1H), 7.32 (t, 2H), 7.46-7.57 (m, 2H), 7.65 (d, 2H), 8.00 (s, 1H), 8.06 (s, 1H), 8.25 (s, 1H), 8.32 (s, 1H), 10.10 (s, 1H), 13.03 (br. s., 1H).

MS (ESIpos) m/z=441 [M+H]⁺.

### Reference Example 6 of WO2013/174744(A1)

### 4-(1H-Indazol-5-ylamino)-N-isopropyl-5, 6, 7,8-tetrahydro[1]benzothieno[2,3-d]pyrimidine-7-carboxamide

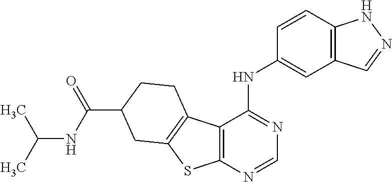 [SMILES: CC(C)NC(=O)C1CCc2c(sc3ncnc(Nc4ccc5[nH]ncc5c4)c23)C1]

To a mixture of 4-(1H-indazol-5-ylamino)-5,6,7,8-tetrahydro[1]benzothieno[2,3-d]pyrimidine-7-carboxylic acid (500 mg) and isopropylamine (443 μL) in N,N-dimethylformamide (14 mL) was added N,N-diisopropylethylamine (1.09 mL), followed by T3P (propylphosphinic anhydride; 3.71 mL of a 50% solution in ethyl acetate), and the mixture was stirred overnight at RT. Water was added, and the supernatant was decanted. The residue was purified by preparative HPLC (Method P1) to give 226 mg of the target compound.

¹H-NMR (300 MHz, DMSO-d₆): δ [ppm]=1.09 (d, 6H), 1.75-1.90 (m, 1H), 2.00-2.14 (m, 1H), 2.56-2.68 (m, 1H), 2.86-2.98 (m, 2H), 3.05-3.28 (m, 1H), 3.80-3.96 (m, 1H), 7.45-7.57 (m, 2H), 7.83 (d, 1H), 7.98 (s, 1H), 8.06 (s, 1H), 8.20 (s, 1H), 8.31 (s, 1H), 13.01 (br. s., 1H).

MS (ESIpos) m/z=407 [M+H]⁺.

### Reference Example 7 of WO2013/174744(A1)

### N-(Cyclopropylmethyl)-4-(1H-indazol-5-ylamino)-5,6,7,8-tetrahydro[1]benzothieno[2,3-d]pyrimidine-7-carboxamide

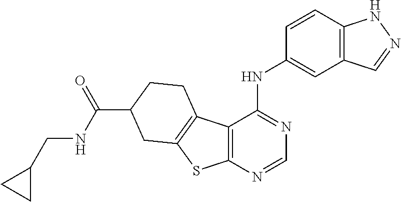 [SMILES: O=C(NCC1CC1)C1CCc2c(sc3ncnc(Nc4ccc5[nH]ncc5c4)c23)C1]

To a mixture of 4-(1H-indazol-5-ylamino)-5,6,7,8-tetrahydro[1]benzothieno[2,3-d]pyrimidine-7-carboxylic acid (500 mg) and cyclopropylmethylamine (451 μL) in N,N-dimethylformamide (14 mL) was added N,N-diisopropylethylamine (1.09 mL), followed by T3P (propylphosphinic anhydride; 3.71 mL of a 50% solution in ethyl acetate), and the mixture was stirred overnight at RT. To drive the reaction to completion, additional portions of cyclopropylmethylamine (451 μL), N,N-diisopropylamine (1.09 mL), and T3P (3.71 mL of a 50% solution in ethyl acetate) were added, and stirring at 60° C. was continued for 4 h. The mixture was added to water, and the precipitated crude product was isolated by filtration to give the target compound (510 mg) in sufficient purity for further processing.

¹H-NMR (300 MHz, DMSO-d₆): δ [ppm]=0.09-0.24 (m, 2H), 0.35-0.49 (m, 2H), 0.81-1.01 (m, 1H), 1.74-1.93 (m, 1H), 2.02-2.16 (m, 1H), 2.61-2.75 (m, 1H), 2.88-3.05 (m, 4H), 3.08-3.28 (m, 2H), 7.44-7.58 (m, 2H), 7.94-8.12 (m, 3H), 8.20 (s, 1H), 8.31 (s, 1H), 13.00 (br. s., 1H).

MS (ESIpos) m/z=419 [M+H]+.

### Reference Example 9 of WO2013/174744(A1)

### 4-(1H-indazol-5-ylamino)-N-[3-(trifluoromethyl)benzyl]-5,6,7,8-tetrahydro[1]benzothieno[2,3-d]pyrimidine-7-carboxamide

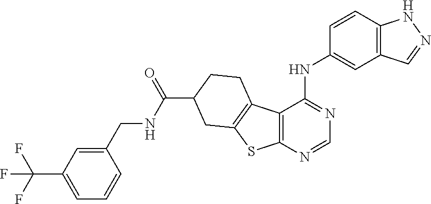 [SMILES: O=C(NCc1cccc(C(F)(F)F)c1)C1CCc2c(sc3ncnc(Nc4ccc5[nH]ncc5c4)c23)C1]

To a mixture of 4-(1H-indazol-5-ylamino)-5,6,7,8-tetrahydro[1]benzothieno[2,3-d]pyrimidine-7-carboxylic acid (500 mg) and 3-(trifluoromethyl)benzylamine (1.14 g) in N,N-dimethylformamide (14 mL) was added N,N-diisopropylethylamine (1.36 mL), followed by T3P (propylphosphinic anhydride; 4.64 mL of a 50% solution in ethyl acetate), and the mixture was stirred at 60° C. for 2 h. After slight concentration in vacuo, the product was stirred with water overnight and the crude product was isolated by filtration. Preparative HPLC (Method P3) gave 510 mg of the target compound.

¹H-NMR (300 MHz, DMSO-d₆): δ [ppm]=1.78-1.96 (m, 1H), 2.08-2.21 (m, 1H), 2.70-2.84 (m, 1H), 2.95-3.04 (m, 2H), 3.09-3.36 (m, 2H, overlaps with water signal), 4.42 (d, 2H), 7.45-7.68 (m, 6H), 7.99 (s, 1H), 8.06 (s, 1H), 8.18-8.26 (m, 1H), 8.31 (s, 1H), 8.67 (t, 1H), 12.98 (br. s, 1H).

MS (ESIpos) m/z=523 [M+H]+.

### Reference Example 66 of WO2013/174744(A1)

### (RS)-4-[(6-Methoxy-1H-indazol-5-yl)amino]-N-(propan-2-yl)-5,6,7,8-tetrahydro[1]benzothieno[2,3-d]pyrimidine-7-carboxamide

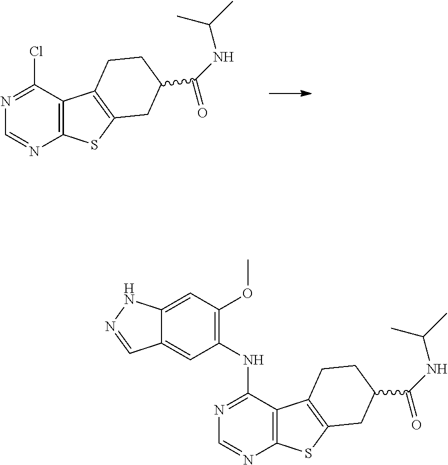 [SMILES: CC(C)NC(=O)C1CCc2c(sc3ncnc(Cl)c23)C1 ; COc1cc2[nH]ncc2cc1Nc1ncnc2sc3c(c12)CCC(C(=O)NC(C)C)C3]

60 mg (194 μmol) (RS)-4-chloro-N-isopropyl-5,6,7,8-tetrahydro[1]benzothieno[2,3-d]pyrimidine-7-carboxamide (prepared according to intermediate example 2a) were transformed in analogy to example 1 using 6-methoxy-1H-indazol-5-amine to give after working up and purification 8.4 mg (9%) of the title compound.

¹H-NMR (DMSO-d₆): δ=1.08 (6H), 1.86 (1H), 2.17 (1H), 2.62 (1H), 2.93 (2H), 3.10 (1H), 3.25 (1H), 3.87 (1H), 3.97 (3H), 7.09 (1H), 7.83 (1H), 7.99 (1H), 8.22 (1H), 8.46 (1H), 8.78 (1H), 12.83 (1H) ppm.

### Pharmaceutical Compositions of the Compounds of the Invention

This invention also relates to pharmaceutical compositions containing one or more compounds of the present invention. These compositions can be utilised to achieve the desired pharmacological effect by administration to a patient in need thereof. A patient, for the purpose of this invention, is a mammal, including a human, in need of treatment for the particular condition or disease. Therefore, the present invention includes pharmaceutical compositions that are comprised of a pharmaceutically acceptable carrier and a pharmaceutically effective amount of a compound, or salt thereof, of the present invention. A pharmaceutically acceptable carrier is preferably a carrier that is relatively non-toxic and innocuous to a patient at concentrations consistent with effective activity of the active ingredient so that any side effects ascribable to the carrier do not vitiate the beneficial effects of the active ingredient. A pharmaceutically effective amount of compound is preferably that amount which produces a result or exerts an influence on the particular condition being treated. The compounds of the present invention can be administered with pharmaceutically-acceptable carriers well known in the art using any effective conventional dosage unit forms, including immediate, slow and timed release preparations, orally, parenterally, topically, nasally, ophthalmically, optically, sublingually, rectally, vaginally, and the like.

For oral administration, the compounds can be formulated into solid or liquid preparations such as capsules, pills, tablets, troches, lozenges, melts, powders, solutions, suspensions, or emulsions, and may be prepared according to methods known to the art for the manufacture of pharmaceutical compositions. The solid unit dosage forms can be a capsule that can be of the ordinary hard- or soft-shelled gelatine type containing, for example, surfactants, lubricants, and inert fillers such as lactose, sucrose, calcium phosphate, and corn starch.

In another embodiment, the compounds of this invention may be tableted with conventional tablet bases such as lactose, sucrose and cornstarch in combination with binders such as acacia, corn starch or gelatine, disintegrating agents intended to assist the break-up and dissolution of the tablet following administration such as potato starch, alginic acid, corn starch, and guar gum, gum tragacanth, acacia, lubricants intended to improve the flow of tablet granulation and to prevent the adhesion of tablet material to the surfaces of the tablet dies and punches, for example talc, stearic acid, or magnesium, calcium or zinc stearate, dyes, colouring agents, and flavouring agents such as peppermint, oil of wintergreen, or cherry flavouring, intended to enhance the aesthetic qualities of the tablets and make them more acceptable to the patient. Suitable excipients for use in oral liquid dosage forms include dicalcium phosphate and diluents such as water and alcohols, for example, ethanol, benzyl alcohol, and polyethylene alcohols, either with or without the addition of a pharmaceutically acceptable surfactant, suspending agent or emulsifying agent. Various other materials may be present as coatings or to otherwise modify the physical form of the dosage unit. For instance tablets, pills or capsules may be coated with shellac, sugar or both.

Dispersible powders and granules are suitable for the preparation of an aqueous suspension. They provide the active ingredient in admixture with a dispersing or wetting agent, a suspending agent and one or more preservatives. Suitable dispersing or wetting agents and suspending agents are exemplified by those already mentioned above. Additional excipients, for example those sweetening, flavouring and colouring agents described above, may also be present.

The pharmaceutical compositions of this invention may also be in the form of oil-in-water emulsions. The oily phase may be a vegetable oil such as liquid paraffin or a mixture of vegetable oils. Suitable emulsifying agents may be (1) naturally occurring gums such as gum acacia and gum tragacanth, (2) naturally occurring phosphatides such as soy bean and lecithin, (3) esters or partial esters derived form fatty acids and hexitol anhydrides, for example, sorbitan monooleate, (4) condensation products of said partial esters with ethylene oxide, for example, polyoxyethylene sorbitan monooleate. The emulsions may also contain sweetening and flavouring agents.

Oily suspensions may be formulated by suspending the active ingredient in a vegetable oil such as, for example, arachis oil, olive oil, sesame oil or coconut oil, or in a mineral oil such as liquid paraffin. The oily suspensions may contain a thickening agent such as, for example, beeswax, hard paraffin, or cetyl alcohol. The suspensions may also contain one or more preservatives, for example, ethyl or n-propyl p-hydroxybenzoate; one or more colouring agents; one or more flavouring agents; and one or more sweetening agents such as sucrose or saccharin.

Syrups and elixirs may be formulated with sweetening agents such as, for example, glycerol, propylene glycol, sorbitol or sucrose. Such formulations may also contain a demulcent, and preservative, such as methyl and propyl parabens and flavouring and colouring agents.

The compounds of this invention may also be administered parenterally, that is, subcutaneously, intravenously, intraocularly, intrasynovially, intramuscularly, or interperitoneally, as injectable dosages of the compound in preferably a physiologically acceptable diluent with a pharmaceutical carrier which can be a sterile liquid or mixture of liquids such as water, saline, aqueous dextrose and related sugar solutions, an alcohol such as ethanol, isopropanol, or hexadecyl alcohol, glycols such as propylene glycol or polyethylene glycol, glycerol ketals such as 2,2-dimethyl-1,1-dioxolane-4-methanol, ethers such as poly(ethylene glycol) 400, an oil, a fatty acid, a fatty acid ester or, a fatty acid glyceride, or an acetylated fatty acid glyceride, with or without the addition of a pharmaceutically acceptable surfactant such as a soap or a detergent, suspending agent such as pectin, carbomers, methylcellulose, hydroxypropylmethylcellulose, or carboxymethylcellulose, or emulsifying agent and other pharmaceutical adjuvants.

Illustrative of oils which can be used in the parenteral formulations of this invention are those of petroleum, animal, vegetable, or synthetic origin, for example, peanut oil, soybean oil, sesame oil, cottonseed oil, corn oil, olive oil, petrolatum and mineral oil. Suitable fatty acids include oleic acid, stearic acid, isostearic acid and myristic acid. Suitable fatty acid esters are, for example, ethyl oleate and isopropyl myristate. Suitable soaps include fatty acid alkali metal, ammonium, and triethanolamine salts and suitable detergents include cationic detergents, for example dimethyl dialkyl ammonium halides, alkyl pyridinium halides, and alkylamine acetates; anionic detergents, for example, alkyl, aryl, and olefin sulfonates, alkyl, olefin, ether, and monoglyceride sulfates, and sulfosuccinates; non-ionic detergents, for example, fatty amine oxides, fatty acid alkanolamides, and poly(oxyethylene-oxypropylene)s or ethylene oxide or propylene oxide copolymers; and amphoteric detergents, for example, alkyl-beta-aminopropionates, and 2-alkylimidazoline quarternary ammonium salts, as well as mixtures.

The parenteral compositions of this invention will typically contain from about 0.5% to about 25% by weight of the active ingredient in solution. Preservatives and buffers may also be used advantageously. In order to minimise or eliminate irritation at the site of injection, such compositions may contain a non-ionic surfactant having a hydrophile-lipophile balance (HLB) preferably of from about 12 to about 17. The quantity of surfactant in such formulation preferably ranges from about 5% to about 15% by weight. The surfactant can be a single component having the above HLB or can be a mixture of two or more components having the desired HLB.

Illustrative of surfactants used in parenteral formulations are the class of polyethylene sorbitan fatty acid esters, for example, sorbitan monooleate and the high molecular weight adducts of ethylene oxide with a hydrophobic base, formed by the condensation of propylene oxide with propylene glycol.

The pharmaceutical compositions may be in the form of sterile injectable aqueous suspensions. Such suspensions may be formulated according to known methods using suitable dispersing or wetting agents and suspending agents such as, for example, sodium carboxymethylcellulose, methylcellulose, hydroxypropylmethyl-cellulose, sodium alginate, polyvinylpyrrolidone, gum tragacanth and gum acacia; dispersing or wetting agents which may be a naturally occurring phosphatide such as lecithin, a condensation product of an alkylene oxide with a fatty acid, for example, polyoxyethylene stearate, a condensation product of ethylene oxide with a long chain aliphatic alcohol, for example, heptadeca-ethyleneoxycetanol, a condensation product of ethylene oxide with a partial ester derived form a fatty acid and a hexitol such as polyoxyethylene sorbitol monooleate, or a condensation product of an ethylene oxide with a partial ester derived from a fatty acid and a hexitol anhydride, for example polyoxyethylene sorbitan monooleate.

The sterile injectable preparation may also be a sterile injectable solution or suspension in a non-toxic parenterally acceptable diluent or solvent. Diluents and solvents that may be employed are, for example, water, Ringer's solution, isotonic sodium chloride solutions and isotonic glucose solutions. In addition, sterile fixed oils are conventionally employed as solvents or suspending media. For this purpose, any bland, fixed oil may be employed including synthetic mono- or diglycerides. In addition, fatty acids such as oleic acid can be used in the preparation of injectables.

A composition of the invention may also be administered in the form of suppositories for rectal administration of the drug. These compositions can be prepared by mixing the drug with a suitable non-irritation excipient which is solid at ordinary temperatures but liquid at the rectal temperature and will therefore melt in the rectum to release the drug. Such materials are, for example, cocoa butter and polyethylene glycol.

Another formulation employed in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art (see, e.g., U.S. Pat. No. 5,023,252, issued Jun. 11, 1991, incorporated herein by reference). Such patches may be constructed for continuous, pulsatile, or on demand delivery of pharmaceutical agents.

Controlled release formulations for parenteral administration include liposomal, polymeric microsphere and polymeric gel formulations that are known in the art.

It may be desirable or necessary to introduce the pharmaceutical composition to the patient via a mechanical delivery device. The construction and use of mechanical delivery devices for the delivery of pharmaceutical agents is well known in the art. Direct techniques for, for example, administering a drug directly to the brain usually involve placement of a drug delivery catheter into the patient's ventricular system to bypass the blood-brain barrier. One such implantable delivery system, used for the transport of agents to specific anatomical regions of the body, is described in U.S. Pat. No. 5,011,472, issued Apr. 30, 1991.

The compositions of the invention can also contain other conventional pharmaceutically acceptable compounding ingredients, generally referred to as carriers or diluents, as necessary or desired. Conventional procedures for preparing such compositions in appropriate dosage forms can be utilized.

Such ingredients and procedures include those described in the following references, each of which is incorporated herein by reference: Powell, M. F. et al., “Compendium of Excipients for Parenteral Formulations” PDA Journal of Pharmaceutical Science a Technology 1998, 52(5), 238-311; Strickley, R. G “Parenteral Formulations of Small Molecule Therapeutics Marketed in the United States (1999)—Part-1” PDA Journal of Pharmaceutical Science a Technology 1999, 53(6), 324-349; and Nema, S. et al., “Excipients and Their Use in Injectable Products” PDA Journal of Pharmaceutical Science a Technology 1997, 51(4), 166-171.

Commonly used pharmaceutical ingredients that can be used as appropriate to formulate the composition for its intended route of administration include:

acidifying agents (examples include but are not limited to acetic acid, citric acid, fumaric acid, hydrochloric acid, nitric acid);

alkalinizing agents (examples include but are not limited to ammonia solution, ammonium carbonate, diethanolamine, monoethanolamine, potassium hydroxide, sodium borate, sodium carbonate, sodium hydroxide, triethanolamine, trolamine);

adsorbents (examples include but are not limited to powdered cellulose and activated charcoal);

aerosol propellants (examples include but are not limited to carbon dioxide, CCl₂F₂, F₂ClC—CClF₂ and CClF₃)

air displacement agents (examples include but are not limited to nitrogen and argon);

antifungal preservatives (examples include but are not limited to benzoic acid, butylparaben, ethylparaben, methylparaben, propylparaben, sodium benzoate);

antimicrobial preservatives (examples include but are not limited to benzalkonium chloride, benzethonium chloride, benzyl alcohol, cetylpyridinium chloride, chlorobutanol, phenol, phenylethyl alcohol, phenylmercuric nitrate and thimerosal);

antioxidants (examples include but are not limited to ascorbic acid, ascorbyl palmitate, butylated hydroxyanisole, butylated hydroxytoluene, hypophosphorus acid, monothioglycerol, propyl gallate, sodium ascorbate, sodium bisulfite, sodium formaldehyde sulfoxylate, sodium metabisulfite);

binding materials (examples include but are not limited to block polymers, natural and synthetic rubber, polyacrylates, polyurethanes, silicones, polysiloxanes and styrene-butadiene copolymers);

buffering agents (examples include but are not limited to potassium metaphosphate, dipotassium phosphate, sodium acetate, sodium citrate anhydrous and sodium citrate dihydrate)

carrying agents (examples include but are not limited to acacia syrup, aromatic syrup, aromatic elixir, cherry syrup, cocoa syrup, orange syrup, syrup, corn oil, mineral oil, peanut oil, sesame oil, bacteriostatic sodium chloride injection and bacteriostatic water for injection)

chelating agents (examples include but are not limited to edetate disodium and edetic acid)

colourants (examples include but are not limited to FD [symbol] C Red No. 3, FD [symbol] C Red No. 20, FD [symbol] C Yellow No. 6, FD [symbol] C Blue No. 2, D [symbol] C Green No. 5, D [symbol] C Orange No. 5, D [symbol] C Red No. 8, caramel and ferric oxide red);

clarifying agents (examples include but are not limited to bentonite);

emulsifying agents (examples include but are not limited to acacia, cetomacrogol, cetyl alcohol, glyceryl monostearate, lecithin, sorbitan monooleate, polyoxyethylene 50 monostearate);

encapsulating agents (examples include but are not limited to gelatin and cellulose acetate phthalate)

flavourants (examples include but are not limited to anise oil, cinnamon oil, cocoa, menthol, orange oil, peppermint oil and vanillin);

humectants (examples include but are not limited to glycerol, propylene glycol and sorbitol);

levigating agents (examples include but are not limited to mineral oil and glycerin);

oils (examples include but are not limited to arachis oil, mineral oil, olive oil, peanut oil, sesame oil and vegetable oil);

ointment bases (examples include but are not limited to lanolin, hydrophilic ointment, polyethylene glycol ointment, petrolatum, hydrophilic petrolatum, white ointment, yellow ointment, and rose water ointment);

penetration enhancers (transdermal delivery) (examples include but are not limited to monohydroxy or polyhydroxy alcohols, mono- or polyvalent alcohols, saturated or unsaturated fatty alcohols, saturated or unsaturated fatty esters, saturated or unsaturated dicarboxylic acids, essential oils, phosphatidyl derivatives, cephalin, terpenes, amides, ethers, ketones and ureas)

plasticizers (examples include but are not limited to diethyl phthalate and glycerol);

solvents (examples include but are not limited to ethanol, corn oil, cottonseed oil, glycerol, isopropanol, mineral oil, oleic acid, peanut oil, purified water, water for injection, sterile water for injection and sterile water for irrigation);

stiffening agents (examples include but are not limited to cetyl alcohol, cetyl esters wax, microcrystalline wax, paraffin, stearyl alcohol, white wax and yellow wax);

suppository bases (examples include but are not limited to cocoa butter and polyethylene glycols (mixtures));

surfactants (examples include but are not limited to benzalkonium chloride, nonoxynol 10, oxtoxynol 9, polysorbate 80, sodium lauryl sulfate and sorbitan mono-palmitate);

suspending agents (examples include but are not limited to agar, bentonite, carbomers, carboxymethylcellulose sodium, hydroxyethyl cellulose, hydroxypropyl cellulose, hydroxypropyl methylcellulose, kaolin, methylcellulose, tragacanth and veegum);

sweetening agents (examples include but are not limited to aspartame, dextrose, glycerol, mannitol, propylene glycol, saccharin sodium, sorbitol and sucrose);

tablet anti-adherents (examples include but are not limited to magnesium stearate and talc);

tablet binders (examples include but are not limited to acacia, alginic acid, carboxymethylcellulose sodium, compressible sugar, ethylcellulose, gelatin, liquid glucose, methylcellulose, non-crosslinked polyvinyl pyrrolidone, and pregelatinized starch);

tablet and capsule diluents (examples include but are not limited to dibasic calcium phosphate, kaolin, lactose, mannitol, microcrystalline cellulose, powdered cellulose, precipitated calcium carbonate, sodium carbonate, sodium phosphate, sorbitol and starch);

tablet coating agents (examples include but are not limited to liquid glucose, hydroxyethyl cellulose, hydroxypropyl cellulose, hydroxypropyl methylcellulose, methylcellulose, ethylcellulose, cellulose acetate phthalate and shellac);

tablet direct compression excipients (examples include but are not limited to dibasic calcium phosphate);

tablet disintegrants (examples include but are not limited to alginic acid, carboxymethylcellulose calcium, microcrystalline cellulose, polacrillin potassium, cross-linked polyvinylpyrrolidone, sodium alginate, sodium starch glycollate and starch);

tablet glidants (examples include but are not limited to colloidal silica, corn starch and talc);

tablet lubricants (examples include but are not limited to calcium stearate, magnesium stearate, mineral oil, stearic acid and zinc stearate);

tablet/capsule opaquants (examples include but are not limited to titanium dioxide);

tablet polishing agents (examples include but are not limited to carnuba wax and white wax);

thickening agents (examples include but are not limited to beeswax, cetyl alcohol and paraffin);

tonicity agents (examples include but are not limited to dextrose and sodium chloride);

viscosity increasing agents (examples include but are not limited to alginic acid, bentonite, carbomers, carboxymethylcellulose sodium, methylcellulose, polyvinyl pyrrolidone, sodium alginate and tragacanth); and

wetting agents (examples include but are not limited to heptadecaethylene oxycetanol, lecithins, sorbitol monooleate, polyoxyethylene sorbitol monooleate, and polyoxyethylene stearate).

Pharmaceutical compositions according to the present invention can be illustrated as follows:

Sterile IV Solution:

A 5 mg/mL solution of the desired compound of this invention can be made using sterile, injectable water, and the pH is adjusted if necessary. The solution is diluted for administration to 1-2 mg/mL with sterile 5% dextrose and is administered as an IV infusion over about 60 minutes.

Lyophilised Powder for IV Administration:

A sterile preparation can be prepared with (i) 100-1000 mg of the desired compound of this invention as a lyophilised powder, (ii) 32-327 mg/mL sodium citrate, and (iii) 300-3000 mg Dextran 40. The formulation is reconstituted with sterile, injectable saline or dextrose 5% to a concentration of 10 to 20 mg/mL, which is further diluted with saline or dextrose 5% to 0.2-0.4 mg/mL, and is administered either IV bolus or by IV infusion over 15-60 minutes.

Intramuscular Suspension:

The following solution or suspension can be prepared, for intramuscular injection:

50 mg/mL of the desired, water-insoluble compound of this invention

5 mg/mL sodium carboxymethylcellulose

4 mg/mL TWEEN 80

9 mg/mL sodium chloride

9 mg/mL benzyl alcohol

Hard Shell Capsules:

A large number of unit capsules are prepared by filling standard two-piece hard galantine capsules each with 100 mg of powdered active ingredient, 150 mg of lactose, 50 mg of cellulose and 6 mg of magnesium stearate.

Soft Gelatin Capsules:

A mixture of active ingredient in a digestible oil such as soybean oil, cottonseed oil or olive oil is prepared and injected by means of a positive displacement pump into molten gelatin to form soft gelatin capsules containing 100 mg of the active ingredient. The capsules are washed and dried. The active ingredient can be dissolved in a mixture of polyethylene glycol, glycerin and sorbitol to prepare a water miscible medicine mix.

Tablets:

A large number of tablets are prepared by conventional procedures so that the dosage unit is 100 mg of active ingredient, 0.2 mg. of colloidal silicon dioxide, 5 mg of magnesium stearate, 275 mg of microcrystalline cellulose, 11 mg. of starch, and 98.8 mg of lactose. Appropriate aqueous and non-aqueous coatings may be applied to increase palatability, improve elegance and stability or delay absorption.

Immediate Release Tablets/Capsules:

These are solid oral dosage forms made by conventional and novel processes. These units are taken orally without water for immediate dissolution and delivery of the medication. The active ingredient is mixed in a liquid containing ingredient such as sugar, gelatin, pectin and sweeteners. These liquids are solidified into solid tablets or caplets by freeze drying and solid state extraction techniques. The drug compounds may be compressed with viscoelastic and thermoelastic sugars and polymers or effervescent components to produce porous matrices intended for immediate release, without the need of water.

Combination Therapies

The term “combination” in the present invention is used as known to persons skilled in the art and may be present as a fixed combination, a non-fixed combination or kit-of-parts.

A “fixed combination” in the present invention is used as known to persons skilled in the art and is defined as a combination wherein the said first active ingredient and the said second active ingredient are present together in one unit dosage or in a single entity. One example of a “fixed combination” is a pharmaceutical composition wherein the said first active ingredient and the said second active ingredient are present in admixture for simultaneous administration, such as in a formulation. Another example of a “fixed combination” is a pharmaceutical combination wherein the said first active ingredient and the said second active ingredient are present in one unit without being in admixture.

A non-fixed combination or “kit-of-parts” in the present invention is used as known to persons skilled in the art and is defined as a combination wherein the said first active ingredient and the said second active ingredient are present in more than one unit. One example of a non-fixed combination or kit-of-parts is a combination wherein the said first active ingredient and the said second active ingredient are present separately. The components of the non-fixed combination or kit-of-parts may be administered separately, sequentially, simultaneously, concurrently or chronologically staggered.

The compounds of this invention can be administered as the sole pharmaceutical agent or in combination with one or more other pharmaceutical agents where the combination causes no unacceptable adverse effects. The present invention relates also to such combinations. For example, the compounds of this invention can be combined with known chemotherapeutic agents or anti-cancer agents, e.g. anti-hyper-proliferative or other indication agents, and the like, as well as with admixtures and combinations thereof. Other indication agents include, but are not limited to, anti-angiogenic agents, mitotic inhibitors, alkylating agents, anti-metabolites, DNA-intercalating antibiotics, growth factor inhibitors, cell cycle inhibitors, enzyme inhibitors, toposisomerase inhibitors, biological response modifiers, or anti-hormones.

The term “(chemotherapeutic) anti-cancer agents”, includes but is not limited to 131I-chTNT, abarelix, abiraterone, aclarubicin, aldesleukin, alemtuzumab, alitretinoin, altretamine, aminoglutethimide, amrubicin, amsacrine, anastrozole, arglabin, arsenic trioxide, asparaginase, azacitidine, basiliximab, BAY 80-6946, BAY 1000394, belotecan, bendamustine, bevacizumab, bexarotene, bicalutamide, bisantrene, bleomycin, bortezomib, buserelin, busulfan, cabazitaxel, calcium folinate, calcium levofolinate, capecitabine, carboplatin, carmofur, carmustine, catumaxomab, celecoxib, celmoleukin, cetuximab, chlorambucil, chlormadinone, chlormethine, cisplatin, cladribine, clodronic acid, clofarabine, crisantaspase, cyclophosphamide, cyproterone, cytarabine, dacarbazine, dactinomycin, darbepoetin alfa, dasatinib, daunorubicin, decitabine, degarelix, denileukin diftitox, denosumab, deslorelin, dibrospidium chloride, docetaxel, doxifluridine, doxorubicin, doxorubicin+estrone, eculizumab, edrecolomab, elliptinium acetate, eltrombopag, endostatin, enocitabine, epirubicin, epitiostanol, epoetin alfa, epoetin beta, eptaplatin, eribulin, erlotinib, estradiol, estramustine, etoposide, everolimus, exemestane, fadrozole, filgrastim, fludarabine, fluorouracil, flutamide, formestane, fotemustine, fulvestrant, gallium nitrate, ganirelix, gefitinib, gemcitabine, gemtuzumab, glutoxim, goserelin, histamine dihydrochloride, histrelin, hydroxycarbamide, 1-125 seeds, ibandronic acid, ibritumomab tiuxetan, idarubicin, ifosfamide, imatinib, imiquimod, improsulfan, interferon alfa, interferon beta, interferon gamma, ipilimumab, irinotecan, ixabepilone, lanreotide, lapatinib, lenalidomide, lenograstim, lentinan, letrozole, leuprorelin, levamisole, lisuride, lobaplatin, lomustine, lonidamine, masoprocol, medroxyprogesterone, megestrol, melphalan, mepitiostane, mercaptopurine, methotrexate, methoxsalen, Methyl aminolevulinate, methyltestosterone, mifamurtide, miltefosine, miriplatin, mitobronitol, mitoguazone, mitolactol, mitomycin, mitotane, mitoxantrone, nedaplatin, nelarabine, nilotinib, nilutamide, nimotuzumab, nimustine, nitracrine, ofatumumab, omeprazole, oprelvekin, oxaliplatin, p53 gene therapy, paclitaxel, palifermin, palladium-103 seed, pamidronic acid, panitumumab, pazopanib, pegaspargase, PEG-epoetin beta (methoxy PEG-epoetin beta), pegfilgrastim, peginterferon alfa-2b, pemetrexed, pentazocine, pentostatin, peplomycin, perfosfamide, picibanil, pirarubicin, plerixafor, plicamycin, poliglusam, polyestradiol phosphate, polysaccharide-K, porfimer sodium, pralatrexate, prednimustine, procarbazine, quinagolide, radium-223 chloride, raloxifene, raltitrexed, ranimustine, razoxane, refametinib, regorafenib, risedronic acid, rituximab, romidepsin, romiplostim, sargramostim, sipuleucel-T, sizofiran, sobuzoxane, sodium glycididazole, sorafenib, streptozocin, sunitinib, talaporfin, tamibarotene, tamoxifen, tasonermin, teceleukin, tegafur, tegafur+gimeracil+oteracil, temoporfin, temozolomide, temsirolimus, teniposide, testosterone, tetrofosmin, thalidomide, thiotepa, thymalfasin, tioguanine, tocilizumab, topotecan, toremifene, tositumomab, trabectedin, trastuzumab, treosulfan, tretinoin, trilostane, triptorelin, trofosfamide, tryptophan, ubenimex, valrubicin, vandetanib, vapreotide, vemurafenib, vinblastine, vincristine, vindesine, vinflunine, vinorelbine, vorinostat, vorozole, yttrium-90 glass microspheres, zinostatin, zinostatin stimalamer, zoledronic acid, zorubicin.

In a preferred embodiment, a compound of general formula (I) as defined herein is administered in combination with one or more inhibitors of the PI3K-AKT-nnTOR pathway. Examples of inhibitors of the mammalian Target of Rapamycin (mTOR) are Afinitor, Votubia (everolimus).

Generally, the use of cytotoxic and/or cytostatic agents in combination with a compound or composition of the present invention will serve to:
(1) yield better efficacy in reducing the growth of a tumor or even eliminate the tumor as compared to administration of either agent alone,
(2) provide for the administration of lesser amounts of the administered chemotherapeutic agents,
(3) provide for a chemotherapeutic treatment that is well tolerated in the patient with fewer deleterious pharmacological complications than observed with single agent chemotherapies and certain other combined therapies,
(4) provide for treating a broader spectrum of different cancer types in mammals, especially humans,
(5) provide for a higher response rate among treated patients,
(6) provide for a longer survival time among treated patients compared to standard chemotherapy treatments,
(7) provide a longer time for tumor progression, and/or
(8) yield efficacy and tolerability results at least as good as those of the agents used alone, compared to known instances where other cancer agent combinations produce antagonistic effects.

Methods of Sensitizing Cells to Radiation

In a distinct embodiment of the present invention, a compound of the present invention may be used to sensitize a cell to radiation. That is, treatment of a cell with a compound of the present invention prior to radiation treatment of the cell renders the cell more susceptible to DNA damage and cell death than the cell would be in the absence of any treatment with a compound of the invention. In one aspect, the cell is treated with at least one compound of the invention.

Thus, the present invention also provides a method of killing a cell, wherein a cell is administered one or more compounds of the invention in combination with conventional radiation therapy.

The present invention also provides a method of rendering a cell more susceptible to cell death, wherein the cell is treated with one or more compounds of the invention prior to the treatment of the cell to cause or induce cell death. In one aspect, after the cell is treated with one or more compounds of the invention, the cell is treated with at least one compound, or at least one method, or a combination thereof, in order to cause DNA damage for the purpose of inhibiting the function of the normal cell or killing the cell.

In one embodiment, a cell is killed by treating the cell with at least one DNA damaging agent. That is, after treating a cell with one or more compounds of the invention to sensitize the cell to cell death, the cell is treated with at least one DNA damaging agent to kill the cell. DNA damaging agents useful in the present invention include, but are not limited to, chemotherapeutic agents (e.g., cisplatinum), ionizing radiation (X-rays, ultraviolet radiation), carcinogenic agents, and mutagenic agents.

In another embodiment, a cell is killed by treating the cell with at least one method to cause or induce DNA damage. Such methods include, but are not limited to, activation of a cell signalling pathway that results in DNA damage when the pathway is activated, inhibiting of a cell signalling pathway that results in DNA damage when the pathway is inhibited, and inducing a biochemical change in a cell, wherein the change results in DNA damage. By way of a non-limiting example, a DNA repair pathway in a cell can be inhibited, thereby preventing the repair of DNA damage and resulting in an abnormal accumulation of DNA damage in a cell.

In one aspect of the invention, a compound of the invention is administered to a cell prior to the radiation or other induction of DNA damage in the cell. In another aspect of the invention, a compound of the invention is administered to a cell concomitantly with the radiation or other induction of DNA damage in the cell. In yet another aspect of the invention, a compound of the invention is administered to a cell immediately after radiation or other induction of DNA damage in the cell has begun.

In another aspect, the cell is in vitro. In another embodiment, the cell is in vivo.

As mentioned supra, the compounds of the present invention have surprisingly been found to effectively inhibit MKNK-1 and may therefore be used for the treatment or prophylaxis of diseases of uncontrolled cell growth, proliferation and/or survival, inappropriate cellular immune responses, or inappropriate cellular inflammatory responses, or diseases which are accompanied with uncontrolled cell growth, proliferation and/or survival, inappropriate cellular immune responses, or inappropriate cellular inflammatory responses, particularly in which the uncontrolled cell growth, proliferation and/or survival, inappropriate cellular immune responses, or inappropriate cellular inflammatory responses is mediated by MKNK-1, such as, for example, haematological tumours, solid tumours, and/or metastases thereof, e.g. leukaemias and myelodysplastic syndrome, malignant lymphomas, head and neck tumours including brain tumours and brain metastases, tumours of the thorax including non-small cell and small cell lung tumours, gastrointestinal tumours, endocrine tumours, mammary and other gynaecological tumours, urological tumours including renal, bladder and prostate tumours, skin tumours, and sarcomas, and/or metastases thereof.

In accordance with another aspect therefore, the present invention covers a compound of general formula (I), or a tautomer, an N-oxide, a hydrate, a solvate, or a salt thereof, particularly a pharmaceutically acceptable salt thereof, or a mixture of same, as described and defined herein, for use in the treatment or prophylaxis of a disease, as mentioned supra.

Another particular aspect of the present invention is therefore the use of a compound of general formula (I), described supra, or a tautomer, an N-oxide, a hydrate, a solvate, or a salt thereof, particularly a pharmaceutically acceptable salt thereof, or a mixture of same, for the prophylaxis or treatment of a disease.

Another particular aspect of the present invention is therefore the use of a compound of general formula (I) described supra for manufacturing a pharmaceutical composition for the treatment or prophylaxis of a disease.

The diseases referred to in the two preceding paragraphs are diseases of uncontrolled cell growth, proliferation and/or survival, inappropriate cellular immune responses, or inappropriate cellular inflammatory responses, or diseases which are accompanied with uncontrolled cell growth, proliferation and/or survival, inappropriate cellular immune responses, or inappropriate cellular inflammatory responses, particularly in which the uncontrolled cell growth, proliferation and/or survival, inappropriate cellular immune responses, or inappropriate cellular inflammatory responses is mediated by MKNK-1, such as, for example, haematological tumours, solid tumours, and/or metastases thereof, e.g. leukaemias and myelodysplastic syndrome, malignant lymphomas, head and neck tumours including brain tumours and brain metastases, tumours of the thorax including non-small cell and small cell lung tumours, gastrointestinal tumours, endocrine tumours, mammary and other gynaecological tumours, urological tumours including renal, bladder and prostate tumours, skin tumours, and sarcomas, and/or metastases thereof.

The term “inappropriate” within the context of the present invention, in particular in the context of “inappropriate cellular immune responses, or inappropriate cellular inflammatory responses”, as used herein, is to be understood as preferably meaning a response which is less than, or greater than normal, and which is associated with, responsible for, or results in, the pathology of said diseases.

Preferably, the use is in the treatment or prophylaxis of diseases, wherein the diseases are haemotological tumours, solid tumours and/or metastases thereof.

Method of Treating Hyper-Proliferative Disorders

The present invention relates to a method for using the compounds of the present invention and compositions thereof, to treat mammalian hyper-proliferative disorders. Compounds can be utilized to inhibit, block, reduce, decrease, etc., cell proliferation and/or cell division, and/or produce apoptosis. This method comprises administering to a mammal in need thereof, including a human, an amount of a compound of this invention, or a pharmaceutically acceptable salt, isomer, polymorph, metabolite, hydrate, solvate or ester thereof; etc. which is effective to treat the disorder. Hyper-proliferative disorders include but are not limited, e.g., psoriasis, keloids, and other hyperplasias affecting the skin, benign prostate hyperplasia (BPH), solid tumours, such as cancers of the breast, respiratory tract, brain, reproductive organs, digestive tract, urinary tract, eye, liver, skin, head and neck, thyroid, parathyroid and their distant metastases. Those disorders also include lymphomas, sarcomas, and leukaemias.

Examples of breast cancer include, but are not limited to invasive ductal carcinoma, invasive lobular carcinoma, ductal carcinoma in situ, and lobular carcinoma in situ.

Examples of cancers of the respiratory tract include, but are not limited to small-cell and non-small-cell lung carcinoma, as well as bronchial adenoma and pleuropulmonary blastoma.

Examples of brain cancers include, but are not limited to brain stem and hypophtalmic glioma, cerebellar and cerebral astrocytoma, medulloblastoma, ependymoma, as well as neuroectodermal and pineal tumour.

Tumours of the male reproductive organs include, but are not limited to prostate and testicular cancer. Tumours of the female reproductive organs include, but are not limited to endometrial, cervical, ovarian, vaginal, and vulvar cancer, as well as sarcoma of the uterus.

Tumours of the digestive tract include, but are not limited to anal, colon, colorectal, oesophageal, gallbladder, gastric, pancreatic, rectal, small-intestine, and salivary gland cancers.

Tumours of the urinary tract include, but are not limited to bladder, penile, kidney, renal pelvis, ureter, urethral and human papillary renal cancers.

Eye cancers include, but are not limited to intraocular melanoma and retinoblastoma.

Examples of liver cancers include, but are not limited to hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to squamous cell carcinoma, Kaposi's sarcoma, malignant melanoma, Merkel cell skin cancer, and non-melanoma skin cancer.

Head-and-neck cancers include, but are not limited to laryngeal, hypopharyngeal, nasopharyngeal, oropharyngeal cancer, lip and oral cavity cancer and squamous cell. Lymphomas include, but are not limited to AIDS-related lymphoma, non-Hodgkin's lymphoma, cutaneous T-cell lymphoma, Burkitt lymphoma, Hodgkin's disease, and lymphoma of the central nervous system.

Sarcomas include, but are not limited to sarcoma of the soft tissue, osteosarcoma, malignant fibrous histiocytoma, lymphosarcoma, and rhabdomyosarcoma.

Leukemias include, but are not limited to acute myeloid leukemia, acute lymphoblastic leukemia, chronic lymphocytic leukemia, chronic myelogenous leukemia, and hairy cell leukemia.

These disorders have been well characterized in humans, but also exist with a similar etiology in other mammals, and can be treated by administering pharmaceutical compositions of the present invention.

The term “treating” or “treatment” as stated throughout this document is used conventionally, e.g., the management or care of a subject for the purpose of combating, alleviating, reducing, relieving, improving the condition of, etc., of a disease or disorder, such as a carcinoma.

Methods of Treating Kinase Disorders

The present invention also provides methods for the treatment of disorders associated with aberrant mitogen extracellular kinase activity, including, but not limited to stroke, heart failure, hepatomegaly, cardiomegaly, diabetes, Alzheimer's disease, cystic fibrosis, symptoms of xenograft rejections, septic shock or asthma.

Effective amounts of compounds of the present invention can be used to treat such disorders, including those diseases (e.g., cancer) mentioned in the Background section above. Nonetheless, such cancers and other diseases can be treated with compounds of the present invention, regardless of the mechanism of action and/or the relationship between the kinase and the disorder.

The phrase “aberrant kinase activity” or “aberrant tyrosine kinase activity,” includes any abnormal expression or activity of the gene encoding the kinase or of the polypeptide it encodes. Examples of such aberrant activity, include, but are not limited to, over-expression of the gene or polypeptide; gene amplification; mutations which produce constitutively-active or hyperactive kinase activity; gene mutations, deletions, substitutions, additions, etc.

The present invention also provides for methods of inhibiting a kinase activity, especially of mitogen extracellular kinase, comprising administering an effective amount of a compound of the present invention, including salts, polymorphs, metabolites, hydrates, solvates, prodrugs (e.g.: esters) thereof, and diastereoisomeric forms thereof. Kinase activity can be inhibited in cells (e.g., in vitro), or in the cells of a mammalian subject, especially a human patient in need of treatment.

Methods of Treating Angiogenic Disorders

The present invention also provides methods of treating disorders and diseases associated with excessive and/or abnormal angiogenesis.

Inappropriate and ectopic expression of angiogenesis can be deleterious to an organism. A number of pathological conditions are associated with the growth of extraneous blood vessels. These include, e.g., diabetic retinopathy, ischemic retinal-vein occlusion, and retinopathy of prematurity [Aiello et al. New Engl. J. Med. 1994, 331, 1480; Peer et al. Lab. Invest. 1995, 72, 638], age-related macular degeneration [AMD; see, Lopez et al. Invest. Opththalmol. Vis. Sci. 1996, 37, 855], neovascular glaucoma, psoriasis, retrolental fibroplasias, angiofibroma, inflammation, rheumatoid arthritis (RA), restenosis, in-stent restenosis, vascular graft restenosis, etc. In addition, the increased blood supply associated with cancerous and neoplastic tissue, encourages growth, leading to rapid tumour enlargement and metastasis. Moreover, the growth of new blood and lymph vessels in a tumour provides an escape route for renegade cells, encouraging metastasis and the consequence spread of the cancer. Thus, compounds of the present invention can be utilized to treat and/or prevent any of the aforementioned angiogenesis disorders, e.g., by inhibiting and/or reducing blood vessel formation; by inhibiting, blocking, reducing, decreasing, etc. endothelial cell proliferation or other types involved in angiogenesis, as well as causing cell death or apoptosis of such cell types.

Dose and Administration

Based upon standard laboratory techniques known to evaluate compounds useful for the treatment of hyper-proliferative disorders and angiogenic disorders, by standard toxicity tests and by standard pharmacological assays for the determination of treatment of the conditions identified above in mammals, and by comparison of these results with the results of known medicaments that are used to treat these conditions, the effective dosage of the compounds of this invention can readily be determined for treatment of each desired indication. The amount of the active ingredient to be administered in the treatment of one of these conditions can vary widely according to such considerations as the particular compound and dosage unit employed, the mode of administration, the period of treatment, the age and sex of the patient treated, and the nature and extent of the condition treated.

The total amount of the active ingredient to be administered will generally range from about 0.001 mg/kg to about 200 mg/kg body weight per day, and preferably from about 0.01 mg/kg to about 20 mg/kg body weight per day. Clinically useful dosing schedules will range from one to three times a day dosing to once every four weeks dosing. In addition, “drug holidays” in which a patient is not dosed with a drug for a certain period of time, may be beneficial to the overall balance between pharmacological effect and tolerability. A unit dosage may contain from about 0.5 mg to about 1500 mg of active ingredient, and can be administered one or more times per day or less than once a day. The average daily dosage for administration by injection, including intravenous, intramuscular, subcutaneous and parenteral injections, and use of infusion techniques will preferably be from 0.01 to 200 mg/kg of total body weight. The average daily rectal dosage regimen will preferably be from 0.01 to 200 mg/kg of total body weight. The average daily vaginal dosage regimen will preferably be from 0.01 to 200 mg/kg of total body weight. The average daily topical dosage regimen will preferably be from 0.1 to 200 mg administered between one to four times daily. The transdermal concentration will preferably be that required to maintain a daily dose of from 0.01 to 200 mg/kg. The average daily inhalation dosage regimen will preferably be from 0.01 to 100 mg/kg of total body weight.

Of course the specific initial and continuing dosage regimen for each patient will vary according to the nature and severity of the condition as determined by the attending diagnostician, the activity of the specific compound employed, the age and general condition of the patient, time of administration, route of administration, rate of excretion of the drug, drug combinations, and the like. The desired mode of treatment and number of doses of a compound of the present invention or a pharmaceutically acceptable salt or ester or composition thereof can be ascertained by those skilled in the art using conventional treatment tests.

Preferably, the diseases of said method are haematological tumours, solid tumour and/or metastases thereof.

The compounds of the present invention can be used in particular in therapy and prevention, i.e. prophylaxis, of tumour growth and metastases, especially in solid tumours of all indications and stages with or without pre-treatment of the tumour growth.

Methods of testing for a particular pharmacological or pharmaceutical property are well known to persons skilled in the art.

The example testing experiments described herein serve to illustrate the present invention and the invention is not limited to the examples given.

Biological Assays

Examples were tested in selected biological assays one or more times. When tested more than once, data are reported as either average values or as median values, wherein
the average value, also referred to as the arithmetic mean value, represents the sum of the values obtained divided by the number of times tested, and
the median value represents the middle number of the group of values when ranked in ascending or descending order. If the number of values in the data set is odd, the median is the middle value. If the number of values in the data set is even, the median is the arithmetic mean of the two middle values.

Examples were synthesized one or more times. When synthesized more than once, data from biological assays represent average values or median values calculated utilizing data sets obtained from testing of one or more synthetic batch.

Assay Descriptions [symbol] Biological Data

MKNK1 Kinase Assay

MKNK1-inhibitory activity of compounds of the present invention was quantified employing the MKNK1 TR-FRET assay as described in the following paragraphs.

A recombinant fusion protein of Glutathione-S-Transferase (GST, N-terminally) and human full-length MKNK1 (amino acids 1-424 and T344D of accession number BAA 19885.1), expressed in insect cells using baculovirus expression system and purified via glutathione sepharose affinity chromatography, was purchased from Carna Biosciences (product no 02-145) and used as enzyme. As substrate for the kinase reaction the biotinylated peptide biotin-Ahx-IKKRKLTRRKSLKG (C-terminus in amide form) was used which can be purchased e.g. form the company Biosyntan (Berlin-Buch, Germany).

For the assay 50 nL of a 100 fold concentrated solution of the test compound in DMSO was pipetted into a black low volume 384 well microtiter plate (Greiner Bio-One, Frickenhausen, Germany), 2 μL of a solution of MKNK1 in aqueous assay buffer [50 mM HEPES pH 7.5, 5 mM MgCl₂, 1.0 mM dithiothreitol, 0.005% (v/v) Nonidet-P40 (Sigma)] was added and the mixture was incubated for 15 min at 22° C. to allow pre-binding of the test compounds to the enzyme before the start of the kinase reaction. Then the kinase reaction was started by the addition of 3 μL of a solution of adenosine-tri-phosphate (ATP, 16.7 μM=>final conc. in the 5 μL assay volume is 10 μM) and substrate (0.1 μM=>final conc. in the 5 μL assay volume is 0.06 μM) in assay buffer and the resulting mixture was incubated for a reaction time of 45 min at 22° C. The concentration of MKNK1 was adjusted depending of the activity of the enzyme lot and was chosen appropriate to have the assay in the linear range, typical concentrations were in the range of 0.05 μg/ml. The reaction was stopped by the addition of 5 μL of a solution of TR-FRET detection reagents (5 nM streptavidine-XL665 [Cisbio Bioassays, Codolet, France] and 1 nM anti-ribosomal protein S6 (pSer236)-antibody from Invitrogen [#44921G] and 1 nM LANCE EU-W1024 labeled ProteinG [Perkin-Elmer, product no. AD0071]) in an aqueous EDTA-solution (100 mM EDTA, 0.1% (w/v) bovine serum albumin in 50 mM HEPES pH 7.5).

The resulting mixture was incubated for 1 h at 22° C. to allow the formation of complex between the phosphorylated biotinylated peptide and the detection reagents. Subsequently the amount of phosphorylated substrate was evaluated by measurement of the resonance energy transfer from the Eu-chelate to the streptavidine-XL. Therefore, the fluorescence emissions at 620 nm and 665 nm after excitation at 350 nm were measured in a TR-FRET reader, e.g. a Rubystar (BMG Labtechnologies, Offenburg, Germany) or a Viewlux (Perkin-Elmer). The ratio of the emissions at 665 nm and at 622 nm was taken as the measure for the amount of phosphorylated substrate. The data were normalised (enzyme reaction without inhibitor=0% inhibition, all other assay components but no enzyme=100% inhibition). Usually the test compounds were tested on the same microtiterplate in 11 different concentrations in the range of 20 μM to 0.1 nM (20 μM, 5.9 μM, 1.7 μM, 0.51 μM, 0.15 μM, 44 nM, 13 nM, 3.8 nM, 1.1 nM, 0.33 nM and 0.1 nM, the dilution series prepared separately before the assay on the level of the 100 fold concentrated solutions in DMSO by serial 1:3.4 dilutions) in duplicate values for each concentration and 1050 values were calculated.

MKNK1 Kinase High ATP Assay

MKNK1-inhibitory activity at high ATP of compounds of the present invention after their preincubation with MKNK1 was quantified employing the TR-FRET-based MKNK1 high ATP assay as described in the following paragraphs.

A recombinant fusion protein of Glutathione-S-Transferase (GST, N-terminally) and human full-length MKNK1 (amino acids 1-424 and T344D of accession number BAA 19885.1), expressed in insect cells using baculovirus expression system and purified via glutathione sepharose affinity chromatography, was purchased from Carna Biosciences (product no 02-145) and used as enzyme. As substrate for the kinase reaction the biotinylated peptide biotin-Ahx-IKKRKLTRRKSLKG (C-terminus in amide form) was used, which can be purchased e.g. from the company Biosyntan (Berlin-Buch, Germany).

For the assay 50 nL of a 100 fold concentrated solution of the test compound in DMSO was pipetted into a black low volume 384 well microtiter plate (Greiner Bio-One, Frickenhausen, Germany), 2 μL of a solution of MKNK1 in aqueous assay buffer [50 mM HEPES pH 7.5, 5 mM MgCl₂, 1.0 mM dithiothreitol, 0.005% (v/v) Nonidet-P40 (Sigma)] was added and the mixture was incubated for 15 min at 22° C. to allow pre-binding of the test compounds to the enzyme before the start of the kinase reaction. Then the kinase reaction was started by the addition of 3 μL of a solution of adenosine-tri-phosphate (ATP, 3.3 mM=>final conc. in the 5 μL assay volume is 2 mM) and substrate (0.1 μM=>final conc. in the 5 μL assay volume is 0.06 μM) in assay buffer and the resulting mixture was incubated for a reaction time of 30 min at 22° C. The concentration of MKNK1 was adjusted depending of the activity of the enzyme lot and was chosen appropriate to have the assay in the linear range, typical concentrations were in the range of 0.003 μg/mL. The reaction was stopped by the addition of 5 μL of a solution of TR-FRET detection reagents (5 nM streptavidine-XL665 [Cisbio Bioassays, Codolet, France] and 1 nM anti-ribosomal protein S6 (pSer236)-antibody from Invitrogen [#44921G] and 1 nM LANCE EU-W1024 labeled ProteinG [Perkin-Elmer, product no. AD0071]) in an aqueous EDTA-solution (100 mM EDTA, 0.1% (w/v) bovine serum albumin in 50 mM HEPES pH 7.5).

The resulting mixture was incubated for 1 h at 22° C. to allow the formation of complex between the phosphorylated biotinylated peptide and the detection reagents. Subsequently the amount of phosphorylated substrate was evaluated by measurement of the resonance energy transfer from the Eu-chelate to the streptavidine-XL. Therefore, the fluorescence emissions at 620 nm and 665 nm after excitation at 350 nm were measured in a TR-FRET reader, e.g. a Rubystar (BMG Labtechnologies, Offenburg, Germany) or a Viewlux (Perkin-Elmer). The ratio of the emissions at 665 nm and at 622 nm was taken as the measure for the amount of phosphorylated substrate. The data were normalised (enzyme reaction without inhibitor=0% inhibition, all other assay components but no enzyme=100% inhibition). Usually the test compounds were tested on the same microtiterplate in 11 different concentrations in the range of 20 μM to 0.1 nM (e.g. 20 μM, 5.9 μM, 1.7 μM, 0.51 μM, 0.15 μM, 44 nM, 13 nM, 3.8 nM, 1.1 nM, 0.33 nM and 0.1 nM, the dilution series prepared separately before the assay on the level of the 100 fold concentrated solutions in DMSO by serial dilutions, the exact concentrations may vary depending on the pipettor used) in duplicate values for each concentration and 1050 values were calculated. Data are presented in Table 1.

TABLE 1

| | MKNK1 |
|---|---|
| Example | IC₅₀ [nM] |
| 1 | 14.3 |
| 2 | 118.0 |
| 3 | 11.0 |
| 4 | 3.5 |
| 5 | 18.3 |
| 6 | 11.0 |
| 7 | 6.0 |
| 8 | 8.3 |
| 9 | 25.6 |
| 10 | 11.1 |
| 11 | 4.0 |
| 12 | 22.7 |
| 13 | 3.2 |
| 14 | 5.5 |
| 15 | 88.5 |
| 16 | 2.4 |
| 17 | 9.4 |
| 18 | 3.9 |
| 19 | 2.5 |
| 20 | 1.0 |
| 21 | 0.4 |
| 22 | 21.4 |
| 23 | 0.5 |
| 24 | 0.2 |
| 25 | 8.3 |
| 26 | 0.2 |
| 27 | 38.9 |
| 28 | 0.3 |
| 29 | 8.7 |
| 30 | 0.3 |
| 31 | 0.8 |
| 32 | 1.0 |
| 33 | 1.5 |
| 34 | 0.5 |
| 35 | 0.7 |
| 36 | 0.5 |
| 37 | 0.3 |
| 38 | 0.3 |
| 39 | 9.4 |
| 40 | 0.2 |
| 41 | 10.5 |
| 42 | 0.3 |
| 43 | 8.9 |
| 44 | 0.2 |
| 45 | 0.2 |
| 46 | 0.1 |
| 47 | 0.5 |
| 48 | 0.4 |
| 49 | 0.1 |
| 50 | 0.1 |
| 51 | 0.1 |
| 52 | 0.2 |
| 53 | 0.3 |
| 54 | 0.4 |
| 55 | 0.1 |
| 56 | 0.3 |
| 57 | 0.7 |
| 58 | 0.3 |
| 59 | 0.2 |
| 60 | 0.2 |
| 61 | 0.1 |
| 62 | 0.1 |
| 63 | 0.2 |
| 64 | 10.5 |
| 65 | 0.6 |
| 66 | 0.4 |
| 67 | 0.3 |
| 68 | 0.3 |
| 69 | 2.5 |
| 70 | 0.4 |
| 71 | 0.9 |
| 72 | 0.7 |
| 73 | 0.3 |
| 74 | 0.2 |
| 75 | 0.3 |
| 76 | 0.4 |
| 77 | 0.1 |
| 78 | 0.3 |
| 79 | 12.1 |
| 80 | 0.3 |
| 81 | 0.3 |
| 82 | 8.1 |
| 83 | 0.3 |
| 84 | 0.7 |
| 85 | 0.6 |
| 86 | 0.6 |
| 87 | 0.3 |
| 88 | 0.4 |
| 89 | 6.0 |
| 90 | 0.6 |
| 91 | 0.1 |
| 92 | 0.2 |
| 93 | 0.1 |
| 94 | 4.8 |
| 95 | 2.3 |
| 96 | 0.7 |
| 97 | 0.3 |
| 98 | 0.2 |
| 99 | 0.3 |
| 100 | 882.0 |
| 101 | 9.1 |
| 102 | 24.5 |
| 103 | 1.3 |
| 104 | 29.5 |
| 105 | 14.1 |
| 106 | 4.6 |
| 107 | 10.0 |
| 108 | 0.6 |
| 109 | 16.5 |
| 110 | 2.3 |
| 111 | 1.4 |
| 112 | 76.3 |
| 113 | 634.0 |
| 114 | 34.2 |
| 115 | 71.5 |
| 116 | 1190.0 |
| 117 | 20.0 |
| 118 | 92.3 |
| 119 | 757.0 |
| 120 | 0.2 |
| 121 | 0.3 |
| 122 | 0.2 |
| 123 | 0.4 |
| 124 | 0.3 |
| 125 | 0.4 |
| 126 | 0.3 |
| 127 | 6.5 |
| 128 | 1.0 |
| 129 | 8.9 |
| 130 | 0.9 |
| 131 | 0.4 |
| 132 | 27.2 |
| 133 | 37.9 |
| 134 | 8.3 |
| 135 | 14.1 |
| 136 | 24.0 |
| 137 | 33.0 |
| 138 | 192.0 |
| 139 | 95.0 |
| 140 | 0.4 |
| 141 | 3.4 |
| 142 | 3.5 |
| 143 | 3.0 |
| 144 | 1.0 |
| 145 | 5.1 |
| 146 | 13.1 |
| 147 | 5.6 |
| 148 | 4.2 |
| 149 | 5.2 |
| 150 | 7.3 |
| 151 | 39.6 |
| 152 | 0.6 |
| 153 | 17.5 |
| 154 | 22.9 |
| 155 | 7.6 |
| 156 | 10.7 |
| 157 | 21.6 |
| 158 | 10.0 |
| 159 | 1.9 |
| 160 | 1.2 |
| 161 | 1.2 |
| 162 | 11.6 |
| 163 | 0.6 |
| 164 | 0.7 |
| 165 | 0.2 |
| 166 | 20.6 |
| 167 | 0.3 |
| 168 | 0.4 |
| 169 | 0.1 |
| 170 | 0.6 |
| 171 | 0.4 |
| 172 | 0.1 |
| 173 | 0.4 |
| 174 | 0.1 |
| 175 | 0.2 |
| 176 | 39.8 |
| 177 | 2.6 |
| 178 | 0.2 |
| 179 | 0.4 |
| 180 | 0.4 |
| 181 | 3.6 |
| 182 | 5.3 |
| 183 | 0.7 |
| 184 | 0.4 |
| 185 | 0.6 |
| 186 | 44.7 |
| 187 | 0.2 |
| 188 | 0.3 |
| 189 | 0.1 |
| 190 | 0.3 |
| 191 | 1.4 |
| 192 | 2.2 |
| 193 | 222.0 |
| 194 | 44.3 |
| 195 | 0.9 |
| 196 | 0.1 |
| 197 | 0.1 |
| 198 | 0.2 |
| 199 | 0.2 |
| 200 | 1.6 |
| 201 | 23.6 |
| 202 | 1.0 |
| 203 | 17.1 |
| 204 | 0.9 |
| 205 | 0.5 |
| 206 | 0.9 |
| 207 | 0.2 |
| 208 | 0.6 |
| 209 | 0.5 |
| 210 | 0.4 |
| 211 | 0.2 |
| 212 | 0.3 |
| 213 | 0.4 |
| 214 | 1.2 |
| 215 | 1.0 |
| 216 | 0.5 |
| 217 | 0.3 |
| 218 | 0.2 |
| 219 | 0.2 |
| 220 | 4.3 |
| 221 | 3.8 |
| 222 | 3.2 |
| 223 | 8.4 |
| 224 | 3.7 |
| 225 | 8.7 |
| 226 | 7.8 |
| 227 | 7.0 |
| 228 | 6.1 |
| 229 | 4.3 |
| 230 | 2.5 |
| 231 | 4.0 |
| 232 | 5.8 |
| 233 | 2.1 |
| 234 | 3.4 |
| 235 | 3.4 |
| 236 | 14.6 |
| 237 | 2.8 |
| 238 | 3.8 |
| 239 | 0.3 |
| 240 | 0.2 |
| 241 | 0.2 |
| 242 | 20.3 |
| 243 | 1.6 |
| 244 | 48.3 |
| 245 | 0.6 |
| 246 | 5.3 |
| 247 | 0.6 |
| 248 | 2.2 |
| 249 | 2.3 |
| 250 | 2.8 |
| 251 | 1.5 |
| 252 | 1.5 |
| 253 | 1.9 |
| 254 | 1.3 |
| 255 | 2.0 |
| 256 | 43.3 |
| 257 | 2.4 |
| 258 | 0.4 |
| 259 | 14.5 |
| 260 | 0.2 |
| 261 | 3.2 |
| 262 | 0.2 |
| 263 | 1.6 |
| 264 | 1.2 |
| 265 | 0.9 |
| 266 | 0.9 |
| 267 | 0.3 |
| 268 | 0.6 |
| 269 | 1.3 |
| 270 | 1.1 |
| 271 | 0.5 |
| 272 | 1.3 |
| 273 | 0.4 |
| 274 | 0.3 |
| 275 | 4.4 |
| 276 | 10.2 |
| 277 | 59.2 |
| 278 | 53.7 |
| 279 | 84.2 |
| 280 | 1.0 |
| 281 | 0.8 |
| 282 | 0.5 |
| 283 | 0.5 |
| 284 | 0.7 |
| 285 | 1.8 |

In Table 2, the MKNK1 inhibitory activity determined as IC₅₀-value in the MKNK1 kinase high ATP assay described supra for the S-enantiomers is compared with the corresponding R-enantiomers for 13 sets of compound pairs. The activity ratio is defined as ratio of the IC₅₀-value of an R-enantiomer and its corresponding S-enantiomer. In all 13 sets the S-enantiomer inhibits the target MKNK1 8-times to 172-times more potent compared to the corresponding R-enantiomer.

As can be seen from the data presented in Table 3 infra, these findings could not be expected.

TABLE 2

| | | | Activity-Ratio MKNK1 |
|---|---|---|---|
| Example | Absolute configuration at amide | MKNK1 IC₅₀ [nM] | $\frac{{IC}_{50} ( R - configuration )}{{IC}_{50} ( S - configuration )}$ |
| 1 | S | 14.3 | 8.3 |
| 2^{#} | R | 118.0 | |
| 14 | S | 5.5 | 16.1 |
| 15^{#} | R | 88.5 | |
| 21 | S | 0.4 | 53.1 |
| 22^{#} | R | 21.4 | |
| 24 | S | 0.2 | 39.1 |
| 25^{#} | R | 8.3 | |
| 26 | S | 0.2 | 172.9 |
| 27^{#} | R | 38.9 | |
| 28 | S | 0.3 | 26.1 |
| 29^{#} | R | 8.7 | |
| 38 | S | 0.3 | 28.1 |
| 39^{#} | R | 9.4 | |
| 40 | S | 0.2 | 45.9 |
| 41^{#} | R | 10.5 | |
| 42 | S | 0.3 | 33.2 |
| 43^{#} | R | 8.9 | |
| 68 | S | 0.3 | 9.0 |
| 69^{#} | R | 2.5 | |
| 78 | S | 0.3 | 42.9 |
| 79^{#} | R | 12.1 | |
| 81 | S | 0.3 | 27.5 |
| 82^{#} | R | 8.1 | |
| 88 | S | 0.4 | 15.0 |
| 89^{#} | R | 6.0 | |

^{#}Reference example of the present patent application

Table 3 compares 6 sets of compounds described in WO2013/174744(A1) bearing a secondary amide moiety for R¹ as depicted in FIG. 1, infra. Each racemic mixture was separated by chiral HPLC and the activity of the pure enantiomers was determined as described for the compounds listed in Table 2, supra. In contrast to the high activity in favour for the S-enantiomer of the compounds in the present invention the highest activity ratio for the compounds listed in Table 3 was only 3.9.

TABLE 3

| | | | Activity Ratio MKNK1 |
|---|---|---|---|
| Example in WO2013/174744 (A1) | Absolute configuration at amide | MKNK1 IC₅₀ [nM] | $\frac{{IC}_{50} ( R or S - configuration )}{{IC}_{50} ( S or R - configuration )}$ |
| 4 | S or R | 6.5 | 1.8 |
| | R or S | 11.7 | |
| 5 | S or R | 4.1 | 3.5 |
| | R or S | 14.0 | |
| 6 | S or R | 7.5 | 2.1 |
| | R or S | 15.6 | |
| 7 | S or R | 4.5 | 2.3 |
| | R or S | 10.5 | |
| 9 | S or R | 1.8 | 3.9 |
| | R or S | 7.2 | |
| 66 | S or R | 0.9 | 1.9 |
| | R or S | 1.7 | |

FIG. 1: Compound Structures Listed in Table 3:

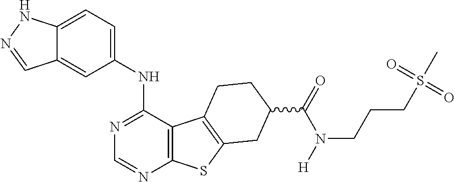 [SMILES: CS(=O)(=O)CCCNC(=O)C1CCc2c(sc3ncnc(Nc4ccc5[nH]ncc5c4)c23)C1]

Example 4 in
WO2013/174744(A1)

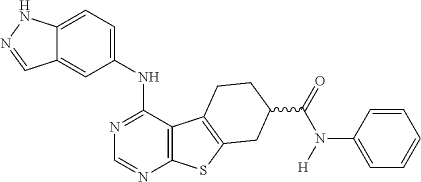 [SMILES: O=C(Nc1ccccc1)C1CCc2c(sc3ncnc(Nc4ccc5[nH]ncc5c4)c23)C1]

Example 5 in
WO2013/174744(A1)

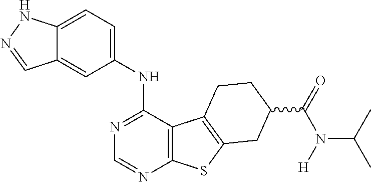 [SMILES: CC(C)NC(=O)C1CCc2c(sc3ncnc(Nc4ccc5[nH]ncc5c4)c23)C1]

Example 6 in
WO2013/174744(A1)

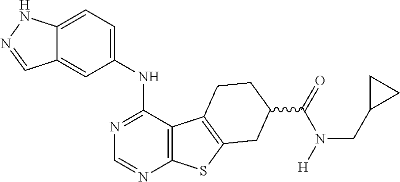 [SMILES: O=C(NCC1CC1)C1CCc2c(sc3ncnc(Nc4ccc5[nH]ncc5c4)c23)C1]

Example 7 in
WO2013/174744(A1)

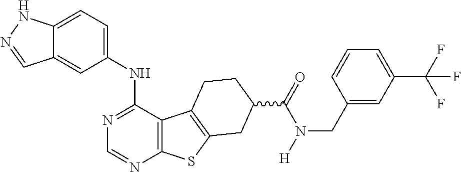 [SMILES: O=C(NCc1cccc(C(F)(F)F)c1)C1CCc2c(sc3ncnc(Nc4ccc5[nH]ncc5c4)c23)C1]

Example 9 in
WO2013/174744(A1)

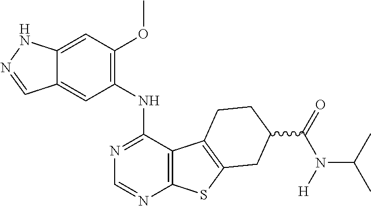 [SMILES: COc1cc2[nH]ncc2cc1Nc1ncnc2sc3c(c12)CCC(C(=O)NC(C)C)C3]

Example 66 in
WO2013/174744(A1)

Table 4 represents the selectivity of the compounds of the present invention for the target kinase MKNK1 compared with a panel of other kinases. The compounds of the present invention show an overall high selectivity for the MKNK1-kinase.

TABLE 4

| | | MW^{c} | |
|---|---|---|---|
| Kinase^{a} | N^{b} | IC₅₀ [nM] | Selectivity^{d} |
| MKNK1 (2 mM ATP) | 173 | 1.6 | 1 |
| Bub1 | 138 | >4970 | >3116 |
| c-kit | 157 | >19280 | >12089 |
| CDK2 | 121 | >19078 | >11963 |
| EGFR | 65 | >12189 | >7643 |
| FGF-R1 | 3 | >20000 | >12541 |
| FLT3 | 155 | >1458 | >914 |
| FLT4 | 146 | >1969 | >1234 |
| FMS (CSF1-R) | 146 | >18613 | >11671 |
| GSK-3-β HTRF | 146 | >19975 | >12525 |
| InsR | 106 | >19936 | >12500 |
| KDR | 150 | >11112 | >6968 |
| Mps1 | 159 | >1192 | >748 |
| Mps1 kinase (2 mM ATP) | 6 | >19812 | >12423 |
| NEK2 | 173 | >19810 | >12422 |
| PDGFR β | 152 | >10246 | >6424 |
| Pim-1 | 108 | >800 | >502 |
| PLK1-Kinase Domain, HTRF | 8 | >20000 | >12541 |
| T-Fyn | 120 | >19200 | >12039 |
| TBK1, HTRF | 53 | >16550 | >10377 |
| Tie 2 | 5 | >20000 | >12541 |

^{a}Panel of kinases tested with the compounds of the present invention. The kinase inhibition data were generated using the protocols described supra for the MKNK kinase and infra for the other kinases; if not stated otherwise, the final ATP-concentration used in the assays was 10 μM.
^{b}N represents the number of compounds of the present invention that have been currently tested at the corresponding kinase. The compounds of the reference examples 2, 15, 22, 25, 27, 29, 39, 41, 43, 69, 79, 82 and 89 have not been included.
^{c}MW represents the geometric mean of the IC₅₀-values [nM] of the compounds of the present invention tested at the corresponding kinase.
^{d}Selectivity is defined as ratio of the MW-IC₅₀ for a distinct kinase and the MW-IC₅₀ for the target kinase MKNK1.

MKNK 2 Kinase High ATP Assay

MKNK 2-inhibitory activity at high ATP of compounds of the present invention after their preincubation with MKNK 2 was quantified employing the TR-FRET-based MKNK 2 high ATP assay as described in the following paragraphs.

A recombinant fusion protein of Glutathione-S-Transferase (GST, N-terminally) and human full-length MKNK 2 (Genbank accession number NP_060042.2), expressed in insect cells using baculovirus expression system, purified via glutathione sepharose affinity chromatography, and activated in vitro with MAPK12, was purchased from Invitrogen (product no PV5608) and used as enzyme. As substrate for the kinase reaction the biotinylated peptide biotin-Ahx-IKKRKLTRRKSLKG (C-terminus in amide form) was used which can be purchased e.g. form the company Biosyntan (Berlin-Buch, Germany).

For the assay 50 nl of a 100 fold concentrated solution of the test compound in DMSO was pipetted into a black low volume 384 well microtiter plate (Greiner Bio-One, Frickenhausen, Germany), 2 μl of a solution of MKNK 2 in aqueous assay buffer [50 mM HEPES pH 7.5, 5 mM MgCl₂, 1.0 mM dithiothreitol, 0.005% (v/v) Nonidet-P40 (G-Biosciences, St. Louis, USA)] was added and the mixture was incubated for 15 min at 22° C. to allow pre-binding of the test compounds to the enzyme before the start of the kinase reaction. Then the kinase reaction was started by the addition of 3 μl of a solution of adenosine-tri-phosphate (ATP, 3.3 mM=>final conc. in the 5 μl assay volume is 2 mM) and substrate (0.1 μM=>final conc. in the 5 μl assay volume is 0.06 μM) in assay buffer and the resulting mixture was incubated for a reaction time of 30 min at 22° C. The concentration of MKNK 2 was adjusted depending of the activity of the enzyme lot and was chosen appropriate to have the assay in the linear range, typical concentrations were in the range of 0.0045 μg/ml. The reaction was stopped by the addition of 5 μl of a solution of TR-FRET detection reagents (5 nM streptavidine-XL665 [Cisbio Bioassays, Codolet, France] and 1 nM anti-ribosomal protein S6 (pSer236)-antibody from Invitrogen [#44921G] and 1 nM LANCE EU-W1024 labeled ProteinG [Perkin-Elmer, product no. AD0071]) in an aqueous EDTA-solution (100 mM EDTA, 0.1% (w/v) bovine serum albumin in 50 mM HEPES pH 7.5).

The resulting mixture was incubated for 1 h at 22° C. to allow the formation of complex between the phosphorylated biotinylated peptide and the detection reagents. Subsequently the amount of phosphorylated substrate was evaluated by measurement of the resonance energy transfer from the Eu-chelate to the streptavidine-XL665. Therefore, the fluorescence emissions at 620 nm and 665 nm after excitation at 350 nm were measured in a TR-FRET reader, e.g. a Pherastar (BMG Labtechnologies, Offenburg, Germany) or a Viewlux (Perkin-Elmer). The ratio of the emissions at 665 nm and at 622 nm was taken as the measure for the amount of phosphorylated substrate. The data were normalised (enzyme reaction without inhibitor=0% inhibition, all other assay components but no enzyme=100% inhibition). Usually the test compounds were tested on the same microtiterplate in 11 different concentrations in the range of 20 μM to 0.1 nM (e.g. 20 μM, 5.9 μM, 1.7 μM, 0.51 μM, 0.15 μM, 44 nM, 13 nM, 3.8 nM, 1.1 nM, 0.33 nM and 0.1 nM, the dilution series prepared separately before the assay on the level of the 100 fold concentrated solutions in DMSO by serial dilutions, the exact concentrations may vary depending on the pipettor used) in duplicate values for each concentration and 1050 values were calculated.

EGFR Kinase Assay

EGFR inhibitory activity of compounds of the present invention was quantified employing the TR-FRET based EGFR assay as described in the following paragraphs.

Epidermal Growth Factor Receptor (EGFR) affinity purified from human carcinoma A431 cells (Sigma-Aldrich, # E3641) was used as kinase. As substrate for the kinase reaction the biotinylated peptide biotin-Ahx-AEEEEYFELVAKKK (C-terminus in amid form) was used which can be purchased e.g. form the company Biosynthan GmbH (Berlin-Buch, Germany).

For the assay 50 nL of a 100 fold concentrated solution of the test compound in DMSO was pipetted into a black low volume 384 well microtiter plate (Greiner Bio-One, Frickenhausen, Germany), 2 μL of a solution of EGFR in aqueous assay [50 mM Hepes/HCl pH 7.0, 1 mM MgCl₂, 5 mM MnCl₂, 0.5 mM activated sodium ortho-vanadate, 0.005% (v/v) Tween-20] were added and the mixture was incubated for 15 min at 22° C. to allow pre-binding of the test compounds to the enzyme before the start of the kinase reaction. Then the kinase reaction was started by the addition of 3 μL of a solution of adenosine-tri-phosphate (ATP, 16.7 μM=>final conc. in the 5 μL assay volume is 10 μM) and substrate (1.67 μM=>final conc. in the 5 μL assay volume is 1 μM) in assay buffer and the resulting mixture was incubated for a reaction time of 30 min at 22° C. The concentration of EGFR was adjusted depending of the activity of the enzyme lot and was chosen appropriate to have the assay in the linear range, typical concentration were in the range of 3 U/ml. The reaction was stopped by the addition of 5 μl of a solution of HTRF detection reagents (0.1 μM streptavidine-XL665 [Cis Biointernational] and 1 nM PT66-Tb-Chelate, an terbium-chelate labelled anti-phospho-tyrosine antibody from Cis Biointernational [instead of the PT66-Tb-chelate PT66-Eu-Cryptate from Perkin Elmer can also be used]) in an aqueous EDTA-solution (80 mM EDTA, 0.2% (w/v) bovine serum albumin in 50 mM HEPES pH 7.5).

The resulting mixture was incubated 1 h at 22° C. to allow the binding of the biotinylated phosphorylated peptide to the streptavidine-XL665 and the PT66-Eu-Chelate. Subsequently the amount of phosphorylated substrate was evaluated by measurement of the resonance energy transfer from the PT66-Eu-Chelate to the streptavidine-XL665. Therefore, the fluorescence emissions at 620 nm and 665 nm after excitation at 337 nm were measured in a HTRF reader, e.g. a Pherastar (BMG Labtechnologies, Offenburg, Germany) or a Viewlux (Perkin-Elmer). The ratio of the emissions at 665 nm and at 622 nm was taken as the measure for the amount of phosphorylated substrate. The data were normalised (enzyme reaction without inhibitor=0% inhibition, all other assay components but no enzyme=100% inhibition). Usually the test compounds were tested on the same microtiterplate in 11 different concentrations in the range of 20 μM to 0.1 nM (e.g. 20 μM, 5.9 μM, 1.7 μM, 0.51 μM, 0.15 μM, 44 nM, 13 nM, 3.8 nM, 1.1 nM, 0.33 nM and 0.1 nM, the dilution series prepared separately before the assay on the level of the 100 fold concentrated solutions in DMSO by serial dilutions, the exact concentrations may vary depending on the pipettor used) in duplicate values for each concentration and IC₅₀ values were calculated.

CDK2/CycE Kinase Assay

CDK2/CycE inhibitory activity of compounds of the present invention can be quantified employing the CDK2/CycE TR-FRET assay as described in the following paragraphs.

Recombinant fusion proteins of GST and human CDK2 and of GST and human CycE, expressed in insect cells (Sf9) and purified by Glutathion-Sepharose affinity chromatography, can be purchased from ProQinase GmbH (Freiburg, Germany). As substrate for the kinase reaction biotinylated peptide biotin-Ttds-YISPLKSPYKISEG (C-terminus in amid form) can be used which can be purchased e.g. from the company JERINI peptide technologies (Berlin, Germany).

For the assay 50 nL of a 100 fold concentrated solution of the test compound in DMSO is pipetted into a black low volume 384 well microtiter plate (Greiner Bio-One, Frickenhausen, Germany), 2 μL of a solution of CDK2/CycE in aqueous assay buffer [50 mM Tris/HCl pH 8.0, 10 mM MgCl₂, 1.0 mM dithiothreitol, 0.1 mM sodium ortho-vanadate, 0.01% (v/v) Nonidet-P40 (Sigma)] are added and the mixture is incubated for 15 min at 22° C. to allow pre-binding of the test compounds to the enzyme before the start of the kinase reaction. Then the kinase reaction is started by the addition of 3 μL of a solution of adenosine-tri-phosphate (ATP, 16.7 μM=>final conc. in the 5 μL assay volume is 10 μM) and substrate (1.25 μM=>final conc. in the 5 μL assay volume is 0.75 μM) in assay buffer and the resulting mixture is incubated for a reaction time of 25 min at 22° C. The concentration of CDK2/CycE is adjusted depending of the activity of the enzyme lot and is chosen appropriate to have the assay in the linear range, typical concentrations ae in the range of 130 ng/ml. The reaction is stopped by the addition of 5 μL of a solution of TR-FRET detection reagents (0.2 μM streptavidine-XL665 [Cisbio Bioassays, Codolet, France] and 1 nM anti-RB (pSer807/pSer811)-antibody from BD Pharmingen [#558389] and 1.2 nM LANCE EU-W1024 labeled anti-mouse IgG antibody [Perkin-Elmer, product no. AD0077, as an alternative a Terbium-cryptate-labeled anti-mouse IgG antibody from Cisbio Bioassays can be used]) in an aqueous EDTA-solution (100 mM EDTA, 0.2% (w/v) bovine serum albumin in 100 mM HEPES/NaOH pH 7.0).

The resulting mixture is incubated 1 h at 22° C. to allow the formation of complex between the phosphorylated biotinylated peptide and the detection reagents. Subsequently the amount of phosphorylated substrate is evaluated by measurement of the resonance energy transfer from the Eu-chelate to the streptavidine-XL. Therefore, the fluorescence emissions at 620 nm and 665 nm after excitation at 350 nm are measured in a TR-FRET reader, e.g. a Rubystar (BMG Labtechnologies, Offenburg, Germany) or a Viewlux (Perkin-Elmer). The ratio of the emissions at 665 nm and at 622 nm are taken as the measure for the amount of phosphorylated substrate. The data are normalised (enzyme reaction without inhibitor=0% inhibition, all other assay components but no enzyme=100% inhibition). Usually the test compounds are tested on the same microtiterplate in 11 different concentrations in the range of 20 μM to 0.1 nM (20 μM, 5.9 μM, 1.7 μM, 0.51 μM, 0.15 μM, 44 nM, 13 nM, 3.8 nM, 1.1 nM, 0.33 nM and 0.1 nM, the dilution series prepared separately before the assay on the level of the 100 fold concentrated solutions in DMSO by serial 1:3.4 dilutions) in duplicate values for each concentration and 1050 values are calculated.

PDGFRβ Kinase Assay

PDGFRβ inhibitory activity of compounds of the present invention can be quantified employing the PDGFRβ HTRF assay as described in the following paragraphs.

As kinase, a GST-His fusion protein containing a C-terminal fragment of human PDGFRβ (amino acids 561-1106, expressed in insect cells [SF9] and purified by affinity chromatography, purchased from Proqinase [Freiburg i.Brsg., Germany] is used. As substrate for the kinase reaction the biotinylated poly-Glu,Tyr (4:1) copolymer (#61GTOBLA) from Cis Biointernational (Marcoule, France) is used.

For the assay 50 nL of a 100 fold concentrated solution of the test compound in DMSO is pipetted into a black low volume 384 well microtiter plate (Greiner Bio-One, Frickenhausen, Germany), 2 μL of a solution of PDGFRβ in aqueous assay buffer [50 mM HEPES/NaOH pH 7.5, 10 mM MgCl₂, 2.5 mM dithiothreitol, 0.01% (v/v) Triton-X100 (Sigma)] are added and the mixture was incubated for 15 min at 22° C. to allow pre-binding of the test compounds to the enzyme before the start of the kinase reaction. Then the kinase reaction is started by the addition of 3 μL of a solution of adenosine-tri-phosphate (ATP, 16.7 μM=>final conc. in the 5 μL assay volume is 10 μM) and substrate (2.27 μg/ml=>final conc. in the 5 μL assay volume is 1.36 μg/ml [˜30 nM]) in assay buffer and the resulting mixture is incubated for a reaction time of 25 min at 22° C. The concentration of PDGFRβ in the assay is adjusted depending of the activity of the enzyme lot and is chosen appropriate to have the assay in the linear range, typical enzyme concentrations are in the range of about 125 pg/μL (final conc. in the 5 μL assay volume). The reaction is stopped by the addition of 5 μL of a solution of HTRF detection reagents (200 nM streptavidine-XLent [Cis Biointernational] and 1.4 nM PT66-Eu-Chelate, an europium-chelate labelled anti-phospho-tyrosine antibody from Perkin Elmer [instead of the PT66-Eu-chelate PT66-Tb-Cryptate from Cis Biointernational can also be used]) in an aqueous EDTA-solution (100 mM EDTA, 0.2% (w/v) bovine serum albumin in 50 mM HEPES/NaOH pH 7.5).

The resulting mixture is incubated 1 h at 22° C. to allow the binding of the biotinylated phosphorylated peptide to the streptavidine-XLent and the PT66-Eu-Chelate. Subsequently the amount of phosphorylated substrate is evaluated by measurement of the resonance energy transfer from the PT66-Eu-Chelate to the streptavidine-XLent. Therefore, the fluorescence emissions at 620 nm and 665 nm after excitation at 350 nm are measured in a HTRF reader, e.g. a Rubystar (BMG Labtechnologies, Offenburg, Germany) or a Viewlux (Perkin-Elmer). The ratio of the emissions at 665 nm and at 622 nm are taken as the measure for the amount of phosphorylated substrate. The data are normalised (enzyme reaction without inhibitor=0% inhibition, all other assay components but no enzyme=100% inhibition). Normally test compound are tested on the same microtiter plate at 10 different concentrations in the range of 20 μM to 1 nM (20 μM, 6.7 μM, 2.2 μM, 0.74 μM, 0.25 μM, 82 nM, 27 nM, 9.2 nM, 3.1 nM and 1 nM, dilution series prepared before the assay at the level of the 100 fold conc. stock solutions by serial 1:3 dilutions) in duplicate values for each concentration and IC₅₀ values are calculated.

Fyn Kinase Assay

C-terminally His6-tagged human recombinant kinase domain of the human T-Fyn expressed in baculovirus infected insect cells (purchased from Invitrogen, P3042) is used as kinase. As substrate for the kinase reaction the biotinylated peptide biotin-KVEKIGEGTYGW (C-terminus in amid form) is used which can be purchased e.g. form the company Biosynthan GmbH (Berlin-Buch, Germany).

For the assay 50 nL of a 100 fold concentrated solution of the test compound in DMSO is pipetted into a black low volume 384 well microtiter plate (Greiner Bio-One, Frickenhausen, Germany), 2 μL of a solution of T-Fyn in aqueous assay buffer [25 mM Tris/HCl pH 7.2, 25 mM MgCl₂, 2 mM dithiothreitol, 0.1% (w/v) bovine serum albumin, 0.03% (v/v) Nonidet-P40 (Sigma)]. are added and the mixture is incubated for 15 min at 22° C. to allow pre-binding of the test compounds to the enzyme before the start of the kinase reaction. Then the kinase reaction is started by the addition of 3 μL of a solution of adenosine-tri-phosphate (ATP, 16.7 μM=>final conc. in the 5 μL assay volume is 10 μM) and substrate (2 μM=>final conc. in the 5 μL assay volume is 1.2 μM) in assay buffer and the resulting mixture is incubated for a reaction time of 60 min at 22° C. The concentration of Fyn is adjusted depending of the activity of the enzyme lot and is chosen appropriate to have the assay in the linear range, typical concentration was 0.13 nM. The reaction is stopped by the addition of 5 μL of a solution of HTRF detection reagents (0.2 μM streptavidine-XL [Cisbio Bioassays, Codolet, France) and 0.66 nM PT66-Eu-Chelate, an europium-chelate labelled anti-phospho-tyrosine antibody from Perkin Elmer [instead of the PT66-Eu-chelate PT66-Tb-Cryptate from Cisbio Bioassays can also be used]) in an aqueous EDTA-solution (125 mM EDTA, 0.2% (w/v) bovine serum albumin in 50 mM HEPES/NaOH pH 7.0).

The resulting mixture is incubated 1 h at 22° C. to allow the binding of the biotinylated phosphorylated peptide to the streptavidine-XL and the PT66-Eu-Chelate. Subsequently the amount of phosphorylated substrate is evaluated by measurement of the resonance energy transfer from the PT66-Eu-Chelate to the streptavidine-XL. Therefore, the fluorescence emissions at 620 nm and 665 nm after excitation at 350 nm are measured in a HTRF reader, e.g. a Rubystar (BMG Labtechnologies, Offenburg, Germany) or a Viewlux (Perkin-Elmer). The ratio of the emissions at 665 nm and at 622 nm are taken as the measure for the amount of phosphorylated substrate. The data are normalised (enzyme reaction without inhibitor=0% inhibition, all other assay components but no enzyme=100% inhibition). Normally test compounds are tested on the same microtiter plate at 10 different concentrations in the range of 20 μM to 1 nM (20 μM, 6.7 μM, 2.2 μM, 0.74 μM, 0.25 μM, 82 nM, 27 nM, 9.2 nM, 3.1 nM and 1 nM, dilution series prepared before the assay at the level of the 100 fold conc. stock solutions by serial 1:3 dilutions) in duplicate values for each concentration and 1050 values are calculated.

Flt4 Kinase Assay

Flt4 inhibitory activity of compounds of the present invention can be quantified employing the Flt4 TR-FRET assay as described in the following paragraphs.

As kinase, a GST-His fusion protein containing a C-terminal fragment of human Flt4 (amino acids 799-1298, expressed in insect cells [SF9] and purified by affinity chromatography, purchased from Proqinase [Freiburg i.Brsg., Germany] is used. As substrate for the kinase reaction the biotinylated peptide Biotin-Ahx-GGEEEEYFELVKKKK (C-terminus in amide form, purchased from Biosyntan, Berlin-Buch, Germany) is used.

For the assay 50 nL of a 100 fold concentrated solution of the test compound in DMSO was pipetted into a black low volume 384 well microtiter plate (Greiner Bio-One, Frickenhausen, Germany), 2 μL of a solution of Flt4 in aqueous assay buffer [25 mM HEPES pH 7.5, 10 mM MgCl₂, 2 mM dithiothreitol, 0.01% (v/v) Triton-X100 (Sigma), 0.5 mM EGTA, and 5 mM β-phospho-glycerol] are added and the mixture is incubated for 15 min at 22° C. to allow pre-binding of the test compounds to the enzyme before the start of the kinase reaction. Then the kinase reaction is started by the addition of 3 μL of a solution of adenosine-tri-phosphate (ATP, 16.7 μM=>final conc. in the 5 μL assay volume is 10 μM) and substrate (1.67 μM=>final conc. in the 5 μL assay volume is 1 μM) in assay buffer and the resulting mixture is incubated for a reaction time of 45 min at 22° C. The concentration of Flt4 in the assay is adjusted depending of the activity of the enzyme lot and was chosen appropriate to have the assay in the linear range, typical enzyme concentrations are in the range of about 120 pg/μL (final conc. in the 5 μL assay volume). The reaction is stopped by the addition of 5 μL of a solution of HTRF detection reagents (200 nM streptavidine-XL665 [Cis Biointernational] and 1 nM PT66-Tb-Cryptate, an terbium-cryptate labelled anti-phospho-tyrosine antibody from Cisbio Bioassays (Codolet, France) in an aqueous EDTA-solution (50 mM EDTA, 0.2% (w/v) bovine serum albumin in 50 mM HEPES pH 7.5).

The resulting mixture is incubated 1 h at 22° C. to allow the binding of the biotinylated phosphorylated peptide to the streptavidine-XL665 and the PT66-Tb-Cryptate. Subsequently the amount of phosphorylated substrate is evaluated by measurement of the resonance energy transfer from the PT66-Tb-Cryptate to the streptavidine-XL665. Therefore, the fluorescence emissions at 620 nm and 665 nm after excitation at 350 nm are measured in a HTRF reader, e.g. a Rubystar (BMG Labtechnologies, Offenburg, Germany) or a Viewlux (Perkin-Elmer). The ratio of the emissions at 665 nm and at 622 nm are taken as the measure for the amount of phosphorylated substrate. The data are normalised (enzyme reaction without inhibitor=0% inhibition, all other assay components but no enzyme=100% inhibition). Normally test compound are tested on the same microtiter plate at 10 different concentrations in the range of 20 μM to 1 nM (20 μM, 6.7 μM, 2.2 μM, 0.74 μM, 0.25 μM, 82 nM, 27 nM, 9.2 nM, 3.1 nM and 1 nM, dilution series prepared before the assay at the level of the 100 fold conc. stock solutions by serial 1:3 dilutions) in duplicate values for each concentration and 1050 values are calculated.

TrkA Kinase Assay

TrkA inhibitory activity of compounds of the present invention can be quantified employing the TrkA HTRF assay as described in the following paragraphs.

As kinase, a GST-His fusion protein containing a C-terminal fragment of human TrkA (amino acids 443-796, expressed in insect cells [SF9] and purified by affinity chromatography, purchased from Proqinase [Freiburg i.Brsg., Germany] is used. As substrate for the kinase reaction the biotinylated poly-Glu,Tyr (4:1) copolymer (#61GTOBLA) from Cis Biointernational (Marcoule, France) is used.

For the assay 50 nL of a 100 fold concentrated solution of the test compound in DMSO is pipetted into a black low volume 384 well microtiter plate (Greiner Bio-One, Frickenhausen, Germany), 2 μL of a solution of TrkA in aqueous assay buffer [8 mM MOPS/HCl pH 7.0, 10 mM MgCl₂, 1 mM dithiothreitol, 0.01% (v/v) NP-40 (Sigma), 0.2 mM EDTA] are added and the mixture was incubated for 15 min at 22° C. to allow pre-binding of the test compounds to the enzyme before the start of the kinase reaction. Then the kinase reaction is started by the addition of 3 μL of a solution of adenosine-tri-phosphate (ATP, 16.7 μM=>final conc. in the 5 μL assay volume is 10 μM) and substrate (2.27 μg/ml=>final conc. in the 5 μL assay volume is 1.36 μg/ml [˜30 nM]) in assay buffer and the resulting mixture is incubated for a reaction time of 60 min at 22° C. The concentration of TrkA in the assay is adjusted depending of the activity of the enzyme lot and is chosen appropriate to have the assay in the linear range, typical enzyme concentrations are in the range of about 20 pg/μL (final conc. in the 5 μL assay volume). The reaction is stopped by the addition of 5 μL of a solution of HTRF detection reagents (30 nM streptavidine-XL665 [Cis Biointernational] and 1.4 nM PT66-Eu-Chelate, an europium-chelate labelled anti-phospho-tyrosine antibody from Perkin Elmer [instead of the PT66-Eu-chelate PT66-Tb-Cryptate from Cis Biointernational can also be used]) in an aqueous EDTA-solution (100 mM EDTA, 0.2% (w/v) bovine serum albumin in 50 mM HEPES/NaOH pH 7.5).

The resulting mixture is incubated 1 h at 22° C. to allow the binding of the biotinylated phosphorylated peptide to the streptavidine-XL665 and the PT66-Eu-Chelate. Subsequently the amount of phosphorylated substrate is evaluated by measurement of the resonance energy transfer from the

PT66-Eu-Chelate to the streptavidine-XL665. Therefore, the fluorescence emissions at 620 nm and 665 nm after excitation at 350 nm are measured in a HTRF reader, e.g. a Rubystar (BMG Labtechnologies, Offenburg, Germany) or a Viewlux (Perkin-Elmer). The ratio of the emissions at 665 nm and at 622 nm are taken as the measure for the amount of phosphorylated substrate. The data are normalised (enzyme reaction without inhibitor=0% inhibition, all other assay components but no enzyme=100% inhibition). Normally test compound are tested on the same microtiter plate at 10 different concentrations in the range of 20 μM to 1 nM (20 μM, 6.7 μM, 2.2 μM, 0.74 μM, 0.25 μM, 82 nM, 27 nM, 9.2 nM, 3.1 nM and 1 nM, dilution series prepared before the assay at the level of the 100 fold conc. stock solutions by serial 1:3 dilutions) in duplicate values for each concentration and 1050 values are calculated.

Bub1 Kinase Assay

Bub1-inhibitory activity of compounds of the present invention can be quantified employing the Bub1 TR-FRET assay as described in the following paragraphs.

N-terminally His6-tagged recombinant catalytic domain of human Bub1 (amino acids 704-1085), expressed in insect cells (Hi5) and purified by Ni-NTA affinity chromatography and subsequent size exclusion chromatography, is used as enzyme. As substrate for the kinase reaction the biotinylated peptide biotin-Ahx-VLLPKKSFAEPG (C-terminus in amid form) is used which can be purchased e.g. form the company Biosyntan (Berlin, Germany).

For the assay 50 nl of a 100 fold concentrated solution of the test compound in DMSO is pipetted into a black low volume 384 well microtiter plate (Greiner Bio-One, Frickenhausen, Germany), 2 μl of a solution of Bub1 in aqueous assay buffer [50 mM Tris/HCl pH 7.5, 10 mM magnesium chloride (MgCl₂), 200 mM potassium chloride (KCl), 1.0 mM dithiothreitol (DTT), 0.1 mM sodium ortho-vanadate, 1% (v/v) glycerol, 0.01% (w/v) bovine serum albumine (BSA), 0.005% (v/v) Trition X-100 (Sigma), 1× Complete EDTA-free protease inhibitor mixture (Roche)] are added and the mixture is incubated for 15 min at 22° C. to allow pre-binding of the test compounds to the enzyme before the start of the kinase reaction. Then the kinase reaction is started by the addition of 3 μl of a solution of adenosine-tri-phosphate (ATP, 16.7 μM=>final conc. in the 5 μl assay volume is 10 μM) and substrate (1.67 μM=>final conc. in the 5 μl assay volume is 1 μM) in assay buffer and the resulting mixture is incubated for a reaction time of 60 min at 22° C. The concentration of Bub1 is adjusted depending of the activity of the enzyme lot and is chosen appropriate to have the assay in the linear range, typical concentrations are in the range of 200 ng/ml. The reaction is stopped by the addition of 5 μl of a solution of TR-FRET detection reagents (0.2 μM streptavidine-XL665 [Cisbio Bioassays, Codolet, France] and 1 nM anti-phosho-Serine antibody [Merck Millipore, cat. #35-002] and 0.4 nM LANCE EU-W1024 labeled anti-mouse IgG antibody [Perkin-Elmer, product no. AD0077, as an alternative a Terbium-cryptate-labeled anti-mouse IgG antibody from Cisbio Bioassays can be used]) in an aqueous EDTA-solution (50 mM EDTA, 0.2% (w/v) bovine serum albumin in 100 mM HEPES pH 7.5).

The resulting mixture is incubated 1 h at 22° C. to allow the formation of complex between the phosphorylated biotinylated peptide and the detection reagents. Subsequently the amount of phosphorylated substrate is evaluated by measurement of the resonance energy transfer from the Eu-chelate to the streptavidine-XL. Therefore, the fluorescence emissions at 620 nm and 665 nm after excitation at 350 nm are measured in a TR-FRET reader, e.g. a Rubystar or Pherastar (both from BMG Labtechnologies, Offenburg, Germany) or a Viewlux (Perkin-Elmer). The ratio of the emissions at 665 nm and at 622 nm are taken as the measure for the amount of phosphorylated substrate. The data are normalised (enzyme reaction without inhibitor=0% inhibition, all other assay components but no enzyme=100% inhibition). Usually the test compounds are tested on the same microtiterplate in 11 different concentrations in the range of 20 μM to 0.1 nM (20 μM, 5.9 μM, 1.7 μM, 0.51 μM, 0.15 μM, 44 nM, 13 nM, 3.8 nM, 1.1 nM, 0.33 nM and 0.1 nM, the dilution series prepared separately before the assay on the level of the 100 fold concentrated solutions in DMSO by serial 1:3.4 dilutions) in duplicate values for each concentration and 1050 values are calculated.

Plk1 Kinase Assay

Plk1-inhibitory activity of compounds of the present invention can be quantified employing the Plk1 TR-FRET assay as described in the following paragraphs.

The recombinant human Plk1 kinase domain (amino acids 33-345) is used as enzyme. To generate this protein, a recombinant fusion protein of a N-terminal GST, a thrombin cleavage site (AAAPFTLVPRGS) and the Plk1 kinase domain is expressed in baculovirus infected insect cells (Hi5) and bound to glutathione-sepharose. After a washing step, the Plk1 kinase domain is released from the glutathione-sepharose by an incubation with thrombin and purified by size-exclusion chromatography. As substrate for the kinase reaction the biotinylated peptide biotin-Ahx-KKLNRTLSFAEPG (C-terminus in amid form) is used which can be purchased e.g. form the company Biosyntan (Berlin, Germany).

For the assay 50 nl of a 100 fold concentrated solution of the test compound in DMSO is pipetted into a black low volume 384 well microtiter plate (Greiner Bio-One, Frickenhausen, Germany), 2 μl of a solution of Plk1 in aqueous assay buffer [50 mM Hepes pH 7.0, 10 mM magnesium chloride (MgCl₂), 1.0 mM dithiothreitol (DTT), 0.05% (w/v) bovine serum albumine (BSA), 0.001% (v/v) Nonidet-P40 (Sigma), 1× Complete EDTA-free protease inhibitor mixture (Roche)] are added and the mixture is incubated for 15 min at 22° C. to allow pre-binding of the test compounds to the enzyme before the start of the kinase reaction. Then the kinase reaction is started by the addition of 3 μl of a solution of adenosine-tri-phosphate (ATP, 16.7 μM=>final conc. in the 5 μl assay volume is 10 μM) and substrate (1.4 μM=>final conc. in the 5 μl assay volume is 0.84 μM) in assay buffer and the resulting mixture is incubated for a reaction time of 60 min at 22° C. The concentration of Plk1 is adjusted depending of the activity of the enzyme lot and is chosen appropriate to have the assay in the linear range, typical concentrations are in the range of 0.5 ng/μl. The reaction is stopped by the addition of 5 μl of a solution of TR-FRET detection reagents (0.4 μM streptavidine-XL665 [Cisbio Bioassays, Codolet, France] and 1 nM anti-phosho-Serine antibody [Merck Millipore, cat. #35-002] and 1.5 nM LANCE EU-W1024 labeled anti-mouse IgG antibody [Perkin-Elmer, product no. AD0077, as an alternative a Terbium-cryptate-labeled anti-mouse IgG antibody from Cisbio Bioassays can be used]) in an aqueous EDTA-solution (100 mM EDTA, 0.12% (w/v) bovine serum albumin in 100 mM HEPES pH 7.5).

The resulting mixture is incubated 1 h at 22° C. to allow the formation of complex between the phosphorylated biotinylated peptide and the detection reagents. Subsequently the amount of phosphorylated substrate is evaluated by measurement of the resonance energy transfer from the Eu-chelate to the streptavidine-XL665. Therefore, the fluorescence emissions at 620 nm and 665 nm after excitation at 350 nm are measured in a TR-FRET reader, e.g. a Rubystar or Pherastar (both from BMG Labtechnologies, Offenburg, Germany) or a Viewlux (Perkin-Elmer). The ratio of the emissions at 665 nm and at 622 nm are taken as the measure for the amount of phosphorylated substrate. The data are normalised (enzyme reaction without inhibitor=0% inhibition, all other assay components but no enzyme=100% inhibition). Usually the test compounds are tested on the same microtiterplate in 11 different concentrations in the range of 20 μM to 0.1 nM (20 μM, 5.9 μM, 1.7 μM, 0.51 μM, 0.15 μM, 44 nM, 13 nM, 3.8 nM, 1.1 nM, 0.33 nM and 0.1 nM, the dilution series prepared separately before the assay on the level of the 100 fold concentrated solutions in DMSO by serial 1:3.4 dilutions) in duplicate values for each concentration and 1050 values are calculated.

Tbk1 Kinase Assay

Tbk1-inhibitory activity of compounds of the present invention can be quantified employing the Tbk1 TR-FRET assay as described in the following paragraphs.

Histidine-tagged recombinant human full length Tbk1, expressed in insect cells and purified by Ni-NTA affinity chromatography is purchased from Life Technologies (product number PR5618B) and used as enzyme. As substrate for the kinase reaction the biotinylated peptide biotin-Ahx-GDEDFSSFAEPG (C-terminus in amid form) is used which can be purchased e.g. form the company Biosyntan (Berlin, Germany).

For the assay 50 nl of a 100 fold concentrated solution of the test compound in DMSO is pipetted into a black low volume 384 well microtiter plate (Greiner Bio-One, Frickenhausen, Germany), 2 μl of a solution of Tbk1 in aqueous assay buffer [50 mM Hepes pH 7.0, 10 mM magnesium chloride (MgCl₂), 1.0 mM dithiothreitol (DTT), 0.05% (w/v) bovine serum albumine (BSA), 0.01% (v/v) Nonidet-P40 (Sigma), 1× Complete EDTA-free protease inhibitor mixture (Roche)] are added and the mixture is incubated for 15 min at 22° C. to allow pre-binding of the test compounds to the enzyme before the start of the kinase reaction. Then the kinase reaction is started by the addition of 3 μl of a solution of adenosine-tri-phosphate (ATP, 16.7 μM=>final conc. in the 5 μl assay volume is 10 μM) and substrate (1.67 μM=>final conc. in the 5 μl assay volume is 1 μM) in assay buffer and the resulting mixture is incubated for a reaction time of 30 min at 22° C. The concentration of Tbk1 is adjusted depending of the activity of the enzyme lot and is chosen appropriate to have the assay in the linear range, typical concentrations are in the range of 0.01 ng/μl. The reaction is stopped by the addition of 5 μl of a solution of TR-FRET detection reagents (0.4 μM streptavidine-XL665 [Cisbio Bioassays, Codolet, France] and 1.5 nM anti-phosho-Serine antibody [Merck Millipore, cat. #35-002] and 0.75 nM LANCE EU-W1024 labeled anti-mouse IgG antibody [Perkin-Elmer, product no. AD0077, as an alternative a Terbium-cryptate-labeled anti-mouse IgG antibody from Cisbio Bioassays can be used]) in an aqueous EDTA-solution (100 mM EDTA, 0.12% (w/v) bovine serum albumin in 100 mM HEPES pH 7.5).

The resulting mixture is incubated 1 h at 22° C. to allow the formation of complex between the phosphorylated biotinylated peptide and the detection reagents. Subsequently the amount of phosphorylated substrate is evaluated by measurement of the resonance energy transfer from the Eu-chelate to the streptavidine-XL665. Therefore, the fluorescence emissions at 620 nm and 665 nm after excitation at 350 nm are measured in a TR-FRET reader, e.g. a Rubystar or Pherastar (both from BMG Labtechnologies, Offenburg, Germany) or a Viewlux (Perkin-Elmer). The ratio of the emissions at 665 nm and at 622 nm are taken as the measure for the amount of phosphorylated substrate. The data are normalised (enzyme reaction without inhibitor=0% inhibition, all other assay components but no enzyme=100% inhibition). Usually the test compounds are tested on the same microtiterplate in 11 different concentrations in the range of 20 μM to 0.1 nM (20 μM, 5.9 μM, 1.7 μM, 0.51 μM, 0.15 μM, 44 nM, 13 nM, 3.8 nM, 1.1 nM, 0.33 nM and 0.1 nM, the dilution series prepared separately before the assay on the level of the 100 fold concentrated solutions in DMSO by serial 1:3.4 dilutions) in duplicate values for each concentration and IC₅₀ values are calculated.

Fms Kinase Assay

Fms-inhibitory activity of compounds of the present invention can be quantified employing the Fms TR-FRET assay as described in the following paragraphs.

A recombinant fusion protein of the C-terminal fragment of human Fms (amino acids 543-972 as in GenBank entry NM_005211.2), and an N-terminal GST-HIS6.-tag, expressed in baculovirus infected Sf9 insect cells and purified by GST-affinity chromatography is purchased from Proqinase (product number 0512-0000-1, CSF1-R) and used as enzyme. As substrate for the kinase reaction the biotinylated peptide biotin-Ahx-GGEEEEYFELVKKKK (C-terminus in amid form) is used which can be purchased e.g. form the company Biosyntan (Berlin, Germany).

For the assay 50 nl of a 100 fold concentrated solution of the test compound in DMSO is pipetted into a black low volume 384 well microtiter plate (Greiner Bio-One, Frickenhausen, Germany), 2 μl of a solution of Fms in aqueous assay buffer [25 mM Hepes pH 7.5, 10 mM magnesium chloride (MgCl₂), 2 mM dithiothreitol (DTT), 5 mM β-glycerophosphate, 0.5 mM EGTA 0.01% (v/v) Trition X-100 (Sigma)] are added and the mixture is incubated for 15 min at 22° C. to allow pre-binding of the test compounds to the enzyme before the start of the kinase reaction. Then the kinase reaction is started by the addition of 3 μl of a solution of adenosine-tri-phosphate (ATP, 16.7 μM=>final conc. in the 5 μl assay volume is 10 μM) and substrate (1.67 μM=>final conc. in the 5 μl assay volume is 1 μM) in assay buffer and the resulting mixture is incubated for a reaction time of 30 min at 22° C. The concentration of Fms is adjusted depending of the activity of the enzyme lot and is chosen appropriate to have the assay in the linear range, typical concentrations are in the range of 0.03 ng/μl. The reaction is stopped by the addition of 5 μl of a solution of TR-FRET detection reagents (0.2 μM streptavidine-XL665 [Cisbio Bioassays, Codolet, France] and 1 nM PT66-Tb-Cryptate, an terbium-cryptate labelled anti-phospho-tyrosine antibody from Cisbio Bioassays [instead of the PT66-Tb-cryptate PT66-Eu-Chelate from Perkin Elmer can also be used]) in an aqueous EDTA-solution (50 mM EDTA, 0.2% (w/v) bovine serum albumin in 50 mM HEPES pH 7.5).

The resulting mixture is incubated 1 h at 22° C. to allow the formation of complex between the phosphorylated biotinylated peptide and the detection reagents. Subsequently the amount of phosphorylated substrate is evaluated by measurement of the resonance energy transfer from the Tb-cryptate to the streptavidine-XL665. Therefore, the fluorescence emissions at 620 nm and 665 nm after excitation at 350 nm are measured in a TR-FRET reader, e.g. a Rubystar or Pherastar (both from BMG Labtechnologies, Offenburg, Germany) or a Viewlux (Perkin-Elmer). The ratio of the emissions at 665 nm and at 622 nm are taken as the measure for the amount of phosphorylated substrate. The data are normalised (enzyme reaction without inhibitor=0% inhibition, all other assay components but no enzyme=100% inhibition). Usually the test compounds are tested on the same microtiterplate in 11 different concentrations in the range of 20 μM to 0.1 nM (20 μM, 5.9 μM, 1.7 μM, 0.51 μM, 0.15 μM, 44 nM, 13 nM, 3.8 nM, 1.1 nM, 0.33 nM and 0.1 nM, the dilution series prepared separately before the assay on the level of the 100 fold concentrated solutions in DMSO by serial 1:3.4 dilutions) in duplicate values for each concentration and 1050 values are calculated.

Pim-1 Kinase Assay

Pim-1-inhibitory activity of compounds of the present invention can be quantified employing the Pim-1 TR-FRET assay as described in the following paragraphs.

N-terminal GST-tagged recombinant human full length Pim-1, expressed in E. coli cells and purified using glutathione agarose is purchased from Millipore (product number 14-573) and used as enzyme. As substrate for the kinase reaction the biotinylated peptide biotin-Ttds-YRRRHLSFAEPG (C-terminus in amid form) is used which can be purchased e.g. form the company Jerini Peptide Technologies (Berlin, Germany).

For the assay 50 nl of a 100 fold concentrated solution of the test compound in DMSO is pipetted into a black low volume 384 well microtiter plate (Greiner Bio-One, Frickenhausen, Germany), 2 μl of a solution of Pim-1 in aqueous assay buffer [25 mM Hepes pH 7.5, 10 mM magnesium chloride (MgCl2), 1.0 mM dithiothreitol (DTT), 0.1 mM sodium ortho-vanadate, 0.01% (w/v) bovine serum albumine (BSA), 0.015% (v/v) Nonidet-P40 (Sigma), 1× Complete EDTA-free protease inhibitor mixture (Roche)] are added and the mixture is incubated for 15 min at 22° C. to allow pre-binding of the test compounds to the enzyme before the start of the kinase reaction. Then the kinase reaction is started by the addition of 3 μl of a solution of adenosine-tri-phosphate (ATP, 16.7 μM=>final conc. in the 5 μl assay volume is 10 μM) and substrate (1.67 μM=>final conc. in the 5 μl assay volume is 1 μM) in assay buffer and the resulting mixture is incubated for a reaction time of 20 min at 22° C. The concentration of Pim-1 is adjusted depending of the activity of the enzyme lot and is chosen appropriate to have the assay in the linear range, typical concentrations are in the range of 0.1 pg/μl. The reaction is stopped by the addition of 5 μl of a solution of TR-FRET detection reagents (0.2 μM streptavidine-XL665 [Cisbio Bioassays, Codolet, France] and 1 nM anti-phosho-Serine antibody [Merck Millipore, cat. #35-002] and 0.5 nM LANCE EU-W1024 labeled anti-mouse IgG antibody [Perkin-Elmer, product no. AD0077, as an alternative a Terbium-cryptate-labeled anti-mouse IgG antibody from Cisbio Bioassays can be used]) in an aqueous EDTA-solution (100 mM EDTA, 0.12% (w/v) bovine serum albumin in 100 mM HEPES pH 7.5).

The resulting mixture is incubated 1 h at 22° C. to allow the formation of complex between the phosphorylated biotinylated peptide and the detection reagents. Subsequently the amount of phosphorylated substrate is evaluated by measurement of the resonance energy transfer from the Eu-chelate to the streptavidine-XL665. Therefore, the fluorescence emissions at 620 nm and 665 nm after excitation at 350 nm are measured in a TR-FRET reader, e.g. a Rubystar or Pherastar (both from BMG Labtechnologies, Offenburg, Germany) or a Viewlux (Perkin-Elmer). The ratio of the emissions at 665 nm and at 622 nm are taken as the measure for the amount of phosphorylated substrate. The data are normalised (enzyme reaction without inhibitor=0% inhibition, all other assay components but no enzyme=100% inhibition). Usually the test compounds are tested on the same microtiterplate in 11 different concentrations in the range of 20 μM to 0.1 nM (20 μM, 5.9 μM, 1.7 μM, 0.51 μM, 0.15 μM, 44 nM, 13 nM, 3.8 nM, 1.1 nM, 0.33 nM and 0.1 nM, the dilution series prepared separately before the assay on the level of the 100 fold concentrated solutions in DMSO by serial 1:3.4 dilutions) in duplicate values for each concentration and 1050 values are calculated.

Flt3 Kinase Assay

Flt3-inhibitory activity of compounds of the present invention can be quantified employing the Flt3 TR-FRET assay as described in the following paragraphs.

N-terminal GST-tagged, recombinant catalytic domain of human Flt3 (amino acids 564-end), expressed in baculovirus infected Sf21 insect cells and purified by glutathione-Sepharose affinity chromatography is purchased from Millipore (catalogue number 14-500) and used as enzyme. As substrate for the kinase reaction the biotinylated peptide biotin-Ahx-GGEEEEYFELVKKKK (C-terminus in amid form) is used which can be purchased e.g. form the company Biosyntan (Berlin, Germany).

For the assay 50 nl of a 100 fold concentrated solution of the test compound in DMSO is pipetted into a black low volume 384 well microtiter plate (Greiner Bio-One, Frickenhausen, Germany), 2 μl of a solution of Flt3 in aqueous assay buffer [25 mM Hepes pH 7.5, 10 mM magnesium chloride (MgCl₂), 2 mM dithiothreitol (DTT), 5 mM β-glycerophosphate, 0.5 mM EGTA 0.01% (v/v) Trition X-100 (Sigma)] are added and the mixture is incubated for 15 min at 22° C. to allow pre-binding of the test compounds to the enzyme before the start of the kinase reaction. Then the kinase reaction is started by the addition of 3 μl of a solution of adenosine-tri-phosphate (ATP, 16.7 μM=>final conc. in the 5 μl assay volume is 10 μM) and substrate (1.67 μM=>final conc. in the 5 μl assay volume is 1 μM) in assay buffer and the resulting mixture is incubated for a reaction time of 45 min at 22° C. The concentration of Flt3 is adjusted depending of the activity of the enzyme lot and is chosen appropriate to have the assay in the linear range, typical concentrations are in the range of 0.2 nM. The reaction is stopped by the addition of 5 μl of a solution of TR-FRET detection reagents (0.2 μM streptavidine-XL665 [Cisbio Bioassays, Codolet, France] and 3 nM PT66-Tb-Cryptate, an terbium-cryptate labelled anti-phospho-tyrosine antibody from Cisbio Bioassays [instead of the PT66-Tb-cryptate PT66-Eu-Chelate from Perkin Elmer can also be used]) in an aqueous EDTA-solution (50 mM EDTA, 0.1% (w/v) bovine serum albumin in 50 mM HEPES pH 7.5).

The resulting mixture is incubated 1 h at 22° C. to allow the formation of complex between the phosphorylated biotinylated peptide and the detection reagents. Subsequently the amount of phosphorylated substrate is evaluated by measurement of the resonance energy transfer from the Tb-cryptate to the streptavidine-XL665. Therefore, the fluorescence emissions at 620 nm and 665 nm after excitation at 350 nm are measured in a TR-FRET reader, e.g. a Rubystar or Pherastar (both from BMG Labtechnologies, Offenburg, Germany) or a Viewlux (Perkin-Elmer). The ratio of the emissions at 665 nm and at 622 nm are taken as the measure for the amount of phosphorylated substrate. The data are normalised (enzyme reaction without inhibitor=0% inhibition, all other assay components but no enzyme=100% inhibition). Usually the test compounds are tested on the same microtiterplate in 11 different concentrations in the range of 20 μM to 0.1 nM (20 μM, 5.9 μM, 1.7 μM, 0.51 μM, 0.15 μM, 44 nM, 13 nM, 3.8 nM, 1.1 nM, 0.33 nM and 0.1 nM, the dilution series prepared separately before the assay on the level of the 100 fold concentrated solutions in DMSO by serial 1:3.4 dilutions) in duplicate values for each concentration and 1050 values are calculated.

FGFR1 Kinase Assay

FGFR1-inhibitory activity of compounds of the present invention after their preincubation with FGFR1 can be quantified employing the TR-FRET-based FGFR1 assay as described in the following paragraphs.

A recombinant fusion protein of Glutathione-S-Transferase (GST, N-terminally), His6-Tag, Thrombin cleavage site, and the intracellular part of human FGFR1 (amino acids 400-800 as in GenBank entry NM_015850), expressed in SF9 insect cells using baculovirus expression system and purified via glutathione sepharose affinity chromatography, is purchased from Proqinase (product no 0101-0000-1) and used as enzyme. As substrate for the kinase reaction the biotinylated peptide biotin-Ahx-AAEEEYFFLFAKKK (C-terminus in amide form) is used which can be purchased e.g. form the company Biosyntan (Berlin-Buch, Germany).

For the assay 50 nl of a 100 fold concentrated solution of the test compound in DMSO is pipetted into a black low volume 384 well microtiter plate (Greiner Bio-One, Frickenhausen, Germany), 2 μl of a solution of FGFR1 in aqueous assay buffer [8 mM MOPS pH 7.0, 10 mM magnesium acetate, 1.0 mM dithiothreitol, 0.05% (w/v) bovine serum albumine (BSA), 0.07% (v/v) Tween-20 (Sigma), 0.2 mM EDTA] is added and the mixture is incubated for 15 min at 22° C. to allow pre-binding of the test compounds to the enzyme before the start of the kinase reaction. Then the kinase reaction is started by the addition of 3 μl of a solution of adenosine-tri-phosphate (ATP, 16 μM=>final conc. in the 5 μl assay volume is 10 μM) and substrate (0.16 μM=>final conc. in the 5 μl assay volume is 0.1 μM) in assay buffer and the resulting mixture is incubated for a reaction time of 60 min at 22° C. The concentration of FGFR1 is adjusted depending of the activity of the enzyme lot and is chosen appropriate to have the assay in the linear range, typical concentrations are in the range of 0.083 μg/ml. The reaction is stopped by the addition of 5 μl of a solution of HTRF detection reagents (25 nM streptavidine-XL665 [Cis Biointernational] and 1 nM PT66-Eu-Chelate, an europium-chelate labelled anti-phospho-tyrosine antibody from Perkin Elmer [instead of the PT66-Eu-chelate PT66-Tb-Cryptate from Cis Biointernational can also be used]) in an aqueous EDTA-solution (50 mM EDTA, 0.1% (w/v) bovine serum albumin in 50 mM HEPES/NaOH pH 7.5).

The resulting mixture is incubated for 1 h at 22° C. to allow the formation of complex between the phosphorylated biotinylated peptide and the detection reagents. Subsequently the amount of phosphorylated substrate is evaluated by measurement of the resonance energy transfer from the Eu-chelate to the streptavidine-XL665. Therefore, the fluorescence emissions at 620 nm and 665 nm after excitation at 350 nm are measured in a TR-FRET reader, e.g. a Rubystar (BMG Labtechnologies, Offenburg, Germany) or a Viewlux (Perkin-Elmer). The ratio of the emissions at 665 nm and at 622 nm are taken as the measure for the amount of phosphorylated substrate. The data are normalised (enzyme reaction without inhibitor=0% inhibition, all other assay components but no enzyme=100% inhibition). Usually the test compounds are tested on the same microtiterplate in 11 different concentrations in the range of 20 μM to 0.1 nM (e.g. 20 μM, 5.9 μM, 1.7 μM, 0.51 μM, 0.15 μM, 44 nM, 13 nM, 3.8 nM, 1.1 nM, 0.33 nM and 0.1 nM, the dilution series prepared separately before the assay on the level of the 100 fold concentrated solutions in DMSO by serial dilutions, the exact concentrations may vary depending on the pipettor used) in duplicate values for each concentration and 1050 values are calculated using an inhouse software.

GSK3β Kinase Assay

GSK3β-inhibitory activity of the compounds of the present invention after their preincubation with GSK3β can be quantified employing the TR-FRET-based GSK3β assay as described in the following paragraphs.

A N-terminal His6-tagged recombinant human full-length GSK3β with the amino acid residue substitution H350L (accession number EMBL L33801), expressed in insect cells using a baculovirus expression system, purified via Ni2+/NTA agarose affinity chromatography, is purchased from Millipore (product no 14-306) and used as enzyme. As substrate for the kinase reaction the biotinylated peptide biotin-Ahx-YRRAAVPPSPSLSRHSSPHQ-pS-EDEEE (C-terminus in amide form) is used which can be purchased e.g. form the company Biosyntan (Berlin-Buch, Germany).

For the assay 50 nl of a 100 fold concentrated solution of the test compound in DMSO is pipetted into a black low volume 384 well microtiter plate (Greiner Bio-One, Frickenhausen, Germany), 2 μl of a solution of GSK3β in aqueous assay buffer [5 mM MOPS pH 6.5, 1 mM MgCl₂, 2.5 mM magnesium acetate, 1.0 mM dithiothreitol, 0.002% (v/v) Nonidet-P40 (G-Biosciences, St. Louis, USA)] is added and the mixture is incubated for 15 min at 22° C. to allow pre-binding of the test compounds to the enzyme before the start of the kinase reaction. Then the kinase reaction is started by the addition of 3 μl of a solution of adenosine-tri-phosphate (ATP, 16.7 μM=>final conc. in the 5 μl assay volume is 10 μM), substrate (1.67 μM=>final conc. in the 5 μl assay volume is 1 μM) and streptavidine-XL665 (0.25 μM=>final conc. in the 5 μl assay volume is 0.15 μM [Cisbio Bioassays, Codolet, France]) in assay buffer and the resulting mixture is incubated for a reaction time of 15 min at 22° C. The concentration of GSK3β is adjusted depending of the activity of the enzyme lot and is chosen appropriate to have the assay in the linear range, a typical concentration is about 0.26 U/ml. The reaction is stopped by the addition of 5 μl of a solution of 6 nM anti-phospho Fos (pS374) antibody (Clone name 34E4, product no. 0118-100/Fos-34E4 from nanoTools Antikorpertechnik in Teningen, Germany), and 0.6 nM LANCE EU-W1024 labeled anti-mouse-IgG antibody [Perkin-Elmer, product no. AD0077]) in an aqueous EDTA-solution (100 mM EDTA, 0.2% (w/v) bovine serum albumin in 100 mM HEPES pH 7.5).

The resulting mixture is incubated for 1 h at 22° C. to allow the formation of complex between the phosphorylated biotinylated peptide and the detection reagents. Subsequently the amount of phosphorylated substrate is evaluated by measurement of the resonance energy transfer from the Eu-chelate to the streptavidine-XL665. Therefore, the fluorescence emissions at 620 nm and 665 nm after excitation at 350 nm are measured in a TR-FRET reader, e.g. a Pherastar (BMG Labtechnologies, Offenburg, Germany) or a Viewlux (Perkin-Elmer). The ratio of the emissions at 665 nm and at 622 nm are taken as the measure for the amount of phosphorylated substrate. The data are normalised (enzyme reaction without inhibitor=0% inhibition, all other assay components but no enzyme=100% inhibition). Usually the test compounds are tested on the same microtiterplate in 11 different concentrations in the range of 20 μM to 0.1 nM (e.g. 20 μM, 5.9 μM, 1.7 μM, 0.51 μM, 0.15 μM, 44 nM, 13 nM, 3.8 nM, 1.1 nM, 0.33 nM and 0.1 nM, the dilution series prepared separately before the assay on the level of the 100 fold concentrated solutions in DMSO by serial dilutions, the exact concentrations may vary depending on the pipettor used) in duplicate values for each concentration and IC₅₀ values are calculated using an inhouse software.

c-Kit Kinase Assay

c-kit-inhibitory activity of compounds of the present invention can be quantified employing the c-kit TR-FRET assay as described in the following paragraphs.

N-terminally GST-HIS6-tagged recombinant human c-kit kinase domain (amino acids 544-976 as in NCBI/Protein entry NP_000213.1), expressed in SF-9 cells, is used as kinase. As substrate for the kinase reaction biotinylated poly-(Glu,Tyr) copolymer (Cisbio Bioassays, France) is used.

For the assay 50 nl of a 100 fold concentrated solution of the test compound in DMSO is pipetted into a black low volume 384 well microtiter plate (Greiner Bio-One, Frickenhausen, Germany), 2 μl of a solution of c-kit in aqueous assay [50 mM Hepes pH 7.0, 1 mM MgCl₂, 5 mM MnCl₂, 1.0 mM dithiothreitol, 0.1 mM activated sodium ortho-vanadate, 0.001% (v/v) Nonidet-P40 (Sigma)]. are added and the mixture is incubated for 15 min at 22° C. to allow pre-binding of the test compounds to the enzyme before the start of the kinase reaction. Then the kinase reaction is started by the addition of 3 μl of a solution of adenosine-tri-phosphate (ATP, 16.7 μM=>final conc. in the 5 μl assay volume is 10 μM) and substrate (0.05 μM=>final conc. in the 5 μl assay volume is 0.03 μM) in assay buffer and the resulting mixture is incubated for a reaction time of 45 min at 22° C. The concentration of c-kit is adjusted depending of the activity of the enzyme lot and is chosen appropriate to have the assay in the linear range. The reaction is stopped by the addition of 5 μl of a solution of TR-FRET detection reagents (0.1 μM streptavidine-XL665 [Cisbio Bioassays, Codolet, France] and 1 nM PT66-Tb-Cryptate, an terbium-cryptate labelled anti-phospho-tyrosine antibody from Cisbio Bioassays [instead of the PT66-Tb-cryptate PT66-Eu-Chelate from Perkin Elmer can also be used]) in an aqueous EDTA-solution (80 mM EDTA, 0.2% (w/v) bovine serum albumin in 50 mM HEPES pH 7.5).

The resulting mixture is incubated 1 h at 22° C. to allow the formation of complex between the phosphorylated biotinylated peptide and the detection reagents. Subsequently the amount of phosphorylated substrate is evaluated by measurement of the resonance energy transfer from the Tb-cryptate to the streptavidine-XL665. Therefore, the fluorescence emissions at 620 nm and 665 nm after excitation at 350 nm are measured in a TR-FRET reader, e.g. a Rubystar or Pherastar (both from BMG Labtechnologies, Offenburg, Germany) or a Viewlux (Perkin-Elmer). The ratio of the emissions at 665 nm and at 622 nm are taken as the measure for the amount of phosphorylated substrate. The data are normalised (enzyme reaction without inhibitor=0% inhibition, all other assay components but no enzyme=100% inhibition). Usually the test compounds are tested on the same microtiterplate in 11 different concentrations in the range of 20 μM to 0.1 nM (20 μM, 5.9 μM, 1.7 μM, 0.51 μM, 0.15 μM, 44 nM, 13 nM, 3.8 nM, 1.1 nM, 0.33 nM and 0.1 nM, the dilution series prepared separately before the assay on the level of the 100 fold concentrated solutions in DMSO by serial 1:3.4 dilutions) in duplicate values for each concentration and 1050 values are calculated.

Nek2 Kinase Assay

Nek2-inhibitory activity of the compounds of the present invention after their preincubation with Nek2 can be quantified employing the TR-FRET-based Nek2 assay as described in the following paragraphs.

A C-terminal His6-tagged recombinant human full-length Nek2 (Genbank accession number NP_002488), expressed in insect cells, purified via Ni2+/NTA agarose affinity chromatography, is purchased from Life Technologies (formerly Invitrogen, product no PV4026) and used as enzyme. As substrate for the kinase reaction the biotinylated peptide biotin-Ahx-HRGLRASFAEPG (C-terminus in amide form) is used which can be purchased e.g. form the company Biosyntan (Berlin-Buch, Germany). For the assay 50 nl of a 100 fold concentrated solution of the test compound in DMSO is pipetted into a black low volume 384 well microtiter plate (Greiner Bio-One, Frickenhausen, Germany), 2 μl of a solution of Nek2 in aqueous assay buffer (50 mM MOPS pH 7.5, 10 mM MgCl₂, 1.0 mM dithiothreitol, 0.1 mM activated sodium ortho-vanadate, 0.01% (w/v) bovine serum albumin, 1× Complete EDTA-free protease inhibitor mixture [Roche]) is added and the mixture is incubated for 15 min at 22° C. to allow pre-binding of the test compounds to the enzyme before the start of the kinase reaction. Then the kinase reaction is started by the addition of 3 μl of a solution of adenosine-tri-phosphate (ATP, 16.7 μM=>final conc. in the 5 μl assay volume is 10 μM), substrate (0.4 μM=>final conc. in the 5 μl assay volume is 0.25 μM) in assay buffer and the resulting mixture is incubated for a reaction time of 30 min at 22° C. The concentration of Nek2 is adjusted depending of the activity of the enzyme lot and is chosen appropriate to have the assay in the linear range, a typical concentration is about 0.06 ng/μl. The reaction is stopped by the addition of 5 μl of a solution of 1.5 nM anti-phosho-Serine antibody [Merck Millipore, “STK antibody”, cat. #35-002], 0.6 nM LANCE EU-W1024 labeled anti-mouse IgG antibody [Perkin-Elmer, product no. AD0077, as an alternative a Terbium-cryptate-labeled anti-mouse IgG antibody from Cisbio Bioassays can be used]), and streptavidine-XL665 (0.125 μM=>final conc. in the 5 μl assay volume is 0.063 μM [Cisbio Bioassays, Codolet, France]) in an aqueous EDTA-solution (50 mM EDTA, 0.1% (w/v) bovine serum albumin in 50 mM HEPES pH 7.5).

The resulting mixture is incubated for 1 h at 22° C. to allow the formation of complex between the phosphorylated biotinylated peptide and the detection reagents. Subsequently the amount of phosphorylated substrate is evaluated by measurement of the resonance energy transfer from the Eu-chelate to the streptavidine-XL665. Therefore, the fluorescence emissions at 620 nm and 665 nm after excitation at 350 nm are measured in a TR-FRET reader, e.g. a Pherastar (BMG Labtechnologies, Offenburg, Germany) or a Viewlux (Perkin-Elmer). The ratio of the emissions at 665 nm and at 622 nm are taken as the measure for the amount of phosphorylated substrate. The data are normalised (enzyme reaction without inhibitor=0% inhibition, all other assay components but no enzyme=100% inhibition). Usually the test compounds are tested on the same microtiterplate in 11 different concentrations in the range of 20 μM to 0.1 nM (e.g. 20 μM, 5.9 μM, 1.7 μM, 0.51 μM, 0.15 μM, 44 nM, 13 nM, 3.8 nM, 1.1 nM, 0.33 nM and 0.1 nM, the dilution series prepared separately before the assay on the level of the 100 fold concentrated solutions in DMSO by serial dilutions, the exact concentrations may vary depending on the pipettor used) in duplicate values for each concentration and IC₅₀ values are calculated.

Ins-R Kinase Assay

Inhibitory activity of compounds against the kinase activity of the insulin receptor can be quantified employing the Ins-R TR-FRET assay as described in the following paragraphs.

A N-terminally GST-tagged recombinant C-terminal fragment (amino acids 989-1382, as in GenBank entry NM_000208) of the human insulin receptor (Ins-R), expressed in SF-9 cells and purified by GST-affinity chromatography, is purchased from ProQinase (Freiburg, Germany) and used as kinase. As substrate for the kinase reaction biotinylated poly-(Glu,Tyr) copolymer (Cis biointernational, France) is used.

For the assay 50 nl of a 100 fold concentrated solution of the test compound in DMSO is pipetted into a black low volume 384 well microtiter plate (Greiner Bio-One, Frickenhausen, Germany), 2 μl of a solution of Ins-R in aqueous assay buffer [50 mM HEPES pH 7.0, 15 mM MnCl₂, 1 mM dithiothreitol, 0.1 mM sodium ortho-vanadate, 0.015% (v/v) PEG20000]. are added and the mixture is incubated for 15 min at 22° C. to allow pre-binding of the test compounds to the enzyme before the start of the kinase reaction. Then the kinase reaction is started by the addition of 3 μl of a solution of adenosine-tri-phosphate (ATP, 16.7 μM=>final conc. in the 5 μl assay volume is 10 μM) and substrate (12 nM=>final conc. in the 5 μl assay volume is 7 nM) in assay buffer and the resulting mixture is incubated for a reaction time of 15 min at 22° C.

The concentration of Ins-R is adjusted depending of the activity of the enzyme lot and is chosen appropriate to have the assay in the linear range, typical concentrations are in the range of 84 pg/μl. The reaction is stopped by the addition of 5 μl of a solution of HTRF detection reagents (0.1 μM streptavidin-XLent [Cisbio Bioassays, Codolet, France] and 1 nM PT66-Tb-Cryptate, a terbium-cryptate labelled anti-phospho-tyrosine antibody from Cisbio Bioassays [instead of the PT66-Tb-cryptate PT66-Eu-Chelate from Perkin Elmer can also be used]) in an aqueous EDTA-solution (80 mM EDTA, 0.2% (w/v) bovine serum albumin in 50 mM HEPES pH 7.5).

The resulting mixture is incubated 1 h at 22° C. to allow the formation of complex between the phosphorylated biotinylated peptide and the detection reagents. Subsequently the amount of phosphorylated substrate is evaluated by measurement of the resonance energy transfer from the Tb-cryptate to the streptavidine-XLent. Therefore, the fluorescence emissions at 620 nm and 665 nm after excitation at 350 nm are measured in a TR-FRET reader, e.g. a Rubystar or Pherastar (both from BMG Labtechnologies, Offenburg, Germany) or a Viewlux (Perkin-Elmer). The ratio of the emissions at 665 nm and at 622 nm are taken as the measure for the amount of phosphorylated substrate. The data are normalised (enzyme reaction without inhibitor=0% inhibition, all other assay components but no enzyme=100% inhibition). Usually the test compounds are tested on the same microtiterplate in 11 different concentrations in the range of 20 μM to 0.1 nM (20 μM, 5.9 μM, 1.7 μM, 0.51 μM, 0.15 μM, 44 nM, 13 nM, 3.8 nM, 1.1 nM, 0.33 nM and 0.1 nM, the dilution series prepared separately before the assay on the level of the 100 fold concentrated solutions in DMSO by serial 1:3.4 dilutions) in duplicate values for each concentration and IC₅₀ values are calculated.

KDR Kinase Assay

KDR-inhibitory activity of compounds of the present invention can be quantified employing the KDR TR-FRET assay as described in the following paragraphs.

A N-terminally GST-tagged recombinant C-terminal fragment (amino acids 807-1356, as in NCBI/Protein entry NP_002244.1) of the human KDR, expressed in SF-9 cells and purified via GST-affinity chromatography, is used as kinase. As substrate for the kinase reaction the biotinylated peptide biotin-Ahx-DFGLARDMYDKEYYSVG (C-terminus in acid form) is used which can be purchased e.g. form the company Biosyntan GmbH (Berlin-Buch, Germany).

For the assay 50 nl of a 100 fold concentrated solution of the test compound in DMSO is pipetted into a black low volume 384 well microtiter plate (Greiner Bio-One, Frickenhausen, Germany), 2 μl of a solution of KDR in aqueous assay buffer [50 mM HEPES pH 7.0, 25 mM MgCl₂, 5 mM MnCl₂, 1.0 mM dithiothreitol, 0.1 mM activated sodium ortho-vanadate, 0.001% (v/v) Nonidet-P40 (Sigma)]. are added and the mixture is incubated for 15 min at 22° C. to allow pre-binding of the test compounds to the enzyme before the start of the kinase reaction. Then the kinase reaction is started by the addition of 3 μl of a solution of adenosine-tri-phosphate (ATP, 16.7 μM=>final conc. in the 5 μl assay volume is 10 μM) and substrate (0.83 μM=>final conc. in the 5 μl assay volume is 0.5 μM) in assay buffer and the resulting mixture is incubated for a reaction time of 45 min at 22° C. The concentration of KDR is adjusted depending of the activity of the enzyme lot and is chosen appropriate to have the assay in the linear range. The reaction is stopped by the addition of 5 μl of a solution of TR-FRET detection reagents (0.08 μM streptavidine-XL665 [Cisbio Bioassays, Codolet, France] and 3 nM PT66-Tb-Cryptate, an terbium-cryptate labelled anti-phospho-tyrosine antibody from Cisbio Bioassays [instead of the PT66-Tb-cryptate PT66-Eu-Chelate from Perkin Elmer can also be used]) in an aqueous EDTA-solution (125 mM EDTA, 0.2% (w/v) bovine serum albumin in 50 mM HEPES pH 7.5).

The resulting mixture is incubated 1 h at 22° C. to allow the formation of complex between the phosphorylated biotinylated peptide and the detection reagents. Subsequently the amount of phosphorylated substrate is evaluated by measurement of the resonance energy transfer from the Tb-cryptate to the streptavidine-XL665. Therefore, the fluorescence emissions at 620 nm and 665 nm after excitation at 350 nm are measured in a TR-FRET reader, e.g. a Rubystar or Pherastar (both from BMG Labtechnologies, Offenburg, Germany) or a Viewlux (Perkin-Elmer). The ratio of the emissions at 665 nm and at 622 nm are taken as the measure for the amount of phosphorylated substrate. The data are normalised (enzyme reaction without inhibitor=0% inhibition, all other assay components but no enzyme=100% inhibition). Usually the test compounds are tested on the same microtiterplate in 11 different concentrations in the range of 20 μM to 0.1 nM (20 μM, 5.9 μM, 1.7 μM, 0.51 μM, 0.15 μM, 44 nM, 13 nM, 3.8 nM, 1.1 nM, 0.33 nM and 0.1 nM, the dilution series prepared separately before the assay on the level of the 100 fold concentrated solutions in DMSO by serial 1:3.4 dilutions) in duplicate values for each concentration and 1050 values are calculated.

Mps-1 Kinase Assay with 2 mM ATP

Mps-1-inhibitory activity of compounds of the present invention at 2 mM ATP can be quantified employing the Mps-1 TR-FRET assay as described in the following paragraphs.

N-terminally GST-tagged human full length recombinant Mps-1 kinase (purchased from Invitrogen, Karslruhe, Germany, cat. no PV4071) is used as kinase. As substrate for the kinase reaction the biotinylated peptide biotin-Ahx-PWDPDDADITEILG (C-terminus in amide form) is used which can be purchased e.g. form the company Biosyntan (Berlin, Germany).

For the assay 50 nl of a 100-fold concentrated solution of the test compound in DMSO is pipetted into a black low volume 384 well microtiter plate (Greiner Bio-One, Frickenhausen, Germany), 2 μl of a solution of Mps-1 in assay buffer [25 mM HEPES pH 7.7, 10 mM MgCl₂, 2 mM DTT, 0.1 mM activated sodium ortho-vanadate, 0.05% (w/v) BSA, 0.001% (v/v) Pluronic F-127] are added and the mixture is incubated for 15 min at 22° C. to allow pre-binding of the test compounds to Mps-1 before the start of the kinase reaction. Then the kinase reaction is started by the addition of 3 μl of a solution of 16.7 μM adenosine-tri-phosphate (ATP, 3.3 mM=>final conc. in the 5 μl assay volume is 2 mM) and peptide substrate (1.67 μM=>final conc. in the 5 μl assay volume is 1 μM) in assay buffer and the resulting mixture is incubated for a reaction time of 60 min at 22° C. The concentration of Mps-1 in the assay is adjusted to the activity of the enzyme lot and is chosen appropriate to have the assay in the linear range, typical enzyme concentrations are in the range of about 0.25 nM (final conc. in the 5 μl assay volume). The reaction is stopped by the addition of 5 μl of a solution of TR-FRET detection reagents (100 mM HEPES pH 7.5, 0.1% BSA, 40 mM EDTA, 140 nM streptavidin-XL665 [Cisbio Bioassays, Codolet, France], 1.5 nM anti-phospho(Ser/Thr)-Europium-antibody [#AD0180, Perkin-Elmer, Germany] (instead of the 1.5 nM anti-phospho(Ser/Thr)-Europium-antibody a mixture of 2 nM unlabeled anti-phospho ser/thr-pro antibody MPM-2 [Millipore cat. #05-368] and 1 nM LANCE EU-W1024 labeled anti-mouse IgG antibody [Perkin-Elmer, product no. AD0077] can be used)).

The resulting mixture is incubated 1 h at 22° C. to allow the binding of the phosphorylated peptide to the anti-phospho(Ser/Thr)-Europium-antibody. Subsequently the amount of phosphorylated substrate is evaluated by measurement of the resonance energy transfer from the Europium-labelled anti-phospho(Ser/Thr) antibody to the Streptavidin-XL665. Therefore, the fluorescence emissions at 620 nm and 665 nm after excitation at 350 nm are measured in a Viewlux TR-FRET reader (Perkin-Elmer, Germany). The “blank-corrected normalized ratio” (a Viewlux specific readout, similar to the traditional ratio of the emissions at 665 nm and at 622 nm, in which blank and Eu-donor crosstalk are subtracted from the 665 nm signal before the ratio is calculated) is taken as the measure for the amount of phosphorylated substrate. The data are normalised (enzyme reaction without inhibitor=0% inhibition, all other assay components but no enzyme=100% inhibition). Usually the test compounds are tested on the same microtiterplate in 11 different concentrations in the range of 20 μM to 0.1 nM (20 μM, 5.9 μM, 1.7 μM, 0.51 μM, 0.15 μM, 44 nM, 13 nM, 3.8 nM, 1.1 nM, 0.33 nM and 0.1 nM, the dilution series prepared separately before the assay on the level of the 100 fold concentrated solutions in DMSO by serial 1:3.4 dilutions) in duplicate values for each concentration and IC₅₀ values are calculated.

Mps-1 Kinase Assay with 10 μM ATP

Mps-1-inhibitory activity of compounds of the present invention at 10 μM ATP can be quantified employing the Mps-1 TR-FRET assay as described in the following paragraphs.

N-terminally GST-tagged human full length recombinant Mps-1 kinase (purchased from Invitrogen, Karslruhe, Germany, cat. no PV4071) is used as kinase. As substrate for the kinase reaction the biotinylated peptide biotin-Ahx-PWDPDDADITEILG (C-terminus in amide form) is used which can be purchased e.g. form the company Biosyntan (Berlin, Germany).

For the assay 50 nl of a 100-fold concentrated solution of the test compound in DMSO is pipetted into a black low volume 384 well microtiter plate (Greiner Bio-One, Frickenhausen, Germany), 2 μl of a solution of Mps-1 in assay buffer [25 mM HEPES pH 7.7, 10 mM MgCl₂, 2 mM DTT, 0.1 mM activated sodium ortho-vanadate, 0.05% (w/v) BSA, 0.001% (v/v) Pluronic F-127] are added and the mixture is incubated for 15 min at 22° C. to allow pre-binding of the test compounds to Mps-1 before the start of the kinase reaction. Then the kinase reaction is started by the addition of 3 μl of a solution of 16.7 μM adenosine-tri-phosphate (ATP, 16.7 μM=>final conc. in the 5 μl assay volume is 10 μM) and peptide substrate (1.67 μM=>final conc. in the 5 μl assay volume is 1 μM) in assay buffer and the resulting mixture is incubated for a reaction time of 60 min at 22° C. The concentration of Mps-1 in the assay is adjusted to the activity of the enzyme lot and is chosen appropriate to have the assay in the linear range, typical enzyme concentrations are in the range of about 0.25 nM (final conc. in the 5 μl assay volume). The reaction is stopped by the addition of 5 μl of a solution of TR-FRET detection reagents (100 mM HEPES pH 7.5, 0.1% BSA, 40 mM EDTA, 140 nM streptavidin-XL665 [Cisbio Bioassays, Codolet, France], 1.5 nM anti-phospho(Ser/Thr)-Europium-antibody [#AD0180, Perkin-Elmer, Germany] (instead of the 1.5 nM anti-phospho(Ser/Thr)-Europium-antibody a mixture of 2 nM unlabeled anti-phospho ser/thr-pro antibody MPM-2 [Millipore cat. #05-368] and 1 nM LANCE EU-W1024 labeled anti-mouse IgG antibody [Perkin-Elmer, product no. AD0077] can be used)).

The resulting mixture is incubated 1 h at 22° C. to allow the binding of the phosphorylated peptide to the anti-phospho(Ser/Thr)-Europium-antibody. Subsequently the amount of phosphorylated substrate is evaluated by measurement of the resonance energy transfer from the Europium-labelled anti-phospho(Ser/Thr) antibody to the Streptavidin-XL665. Therefore, the fluorescence emissions at 620 nm and 665 nm after excitation at 350 nm are measured in a Viewlux TR-FRET reader (Perkin-Elmer, Germany). The “blank-corrected normalized ratio” (a Viewlux specific readout, similar to the traditional ratio of the emissions at 665 nm and at 622 nm, in which blank and Eu-donor crosstalk are subtracted from the 665 nm signal before the ratio is calculated) is taken as the measure for the amount of phosphorylated substrate. The data are normalised (enzyme reaction without inhibitor=0% inhibition, all other assay components but no enzyme=100% inhibition). Usually the test compounds are tested on the same microtiterplate in 11 different concentrations in the range of 20 μM to 0.1 nM (20 μM, 5.9 μM, 1.7 μM, 0.51 μM, 0.15 μM, 44 nM, 13 nM, 3.8 nM, 1.1 nM, 0.33 nM and 0.1 nM, the dilution series prepared separately before the assay on the level of the 100 fold concentrated solutions in DMSO by serial 1:3.4 dilutions) in duplicate values for each concentration and 1050 values are calculated.

Tie-2 Kinase Assay

Tie-2-inhibitory activity of compounds of the present invention can be quantified employing the Tie-2 TR-FRET assay as described in the following paragraphs.

A recombinant fusion protein of GST and the intracellular domains of Tie-2, expressed in insect cells (Hi-5) and purified by glutathione-Sepharose affinity chromatography is used as kinase. Alternatively, commercially available GST-Tie2-fusion protein (Merck Millipore, Dundee, Scotland) can be used. As substrate for the kinase reaction the biotinylated peptide biotin-Ahx-EPKDDAYPLYSDFG (C-terminus in amide form) is used which can be purchased e.g. from the company Biosyntan GmbH (Berlin, Germany).

For the assay 50 nl of a 100 fold concentrated solution of the test compound in DMSO is pipetted into a black low volume 384 well microtiter plate (Greiner Bio-One, Frickenhausen, Germany), 2 μl of a solution of Tie-2 in aqueous assay buffer [50 mM HEPES pH 7.0, 10 mM MgCl₂, 0.5 mM MnCl₂, 1.0 mM dithiothreitol, 0.01% Nonidet-P40, protease inhibitor mixture (“Complete w/o EDTA” from Roche, 1 tablet per 2.5 ml)] are added and the mixture is incubated for 15 min at 22° C. to allow pre-binding of the test compounds to the enzyme before the start of the kinase reaction. Then the kinase reaction is started by the addition of 3 μl of a solution of adenosine-tri-phosphate (ATP, 16.7 μM=>final conc. in the 5 μl assay volume is 10 μM) and substrate peptide (1.67 μM=>final conc. in the 5 μl assay volume is 1 μM) in assay buffer and the resulting mixture is incubated for a reaction time of 60 min at 22° C. The concentration of Tie-2 is adjusted depending of the activity of the enzyme lot and is chosen appropriate to have the assay in the linear range, typical concentrations are in the range of 0.7 ng/μl. The reaction is stopped by the addition of 5 μl of a solution of TR-FRET detection reagents (0.2 μM streptavidine-XLent [Cisbio Bioassays, Codolet, France] and 2 nM PT66-Tb-Cryptate, an terbium-cryptate labelled anti-phospho-tyrosine antibody from Cisbio Bioassays [instead of the PT66-Tb-cryptate PT66-Eu-Chelate from Perkin Elmer can also be used]) in an aqueous EDTA-solution (90 mM EDTA, 0.28% (w/v) bovine serum albumin in 50 mM HEPES pH 7.5).

The resulting mixture is incubated 1 h at 22° C. to allow the formation of complex between the phosphorylated biotinylated peptide and the detection reagents. Subsequently the amount of phosphorylated substrate is evaluated by measurement of the resonance energy transfer from the Tb-cryptate to the streptavidine-XLent. Therefore, the fluorescence emissions at 620 nm and 665 nm after excitation at 350 nm are measured in a TR-FRET reader, e.g. a Rubystar or Pherastar (both from BMG Labtechnologies, Offenburg, Germany) or a Viewlux (Perkin-Elmer). The ratio of the emissions at 665 nm and at 622 nm are taken as the measure for the amount of phosphorylated substrate. The data are normalised (enzyme reaction without inhibitor=0% inhibition, all other assay components but no enzyme=100% inhibition). Usually the test compounds are tested on the same microtiterplate in 11 different concentrations in the range of 20 μM to 0.1 nM (20 μM, 5.9 μM, 1.7 μM, 0.51 μM, 0.15 μM, 44 nM, 13 nM, 3.8 nM, 1.1 nM, 0.33 nM and 0.1 nM, the dilution series prepared separately before the assay on the level of the 100 fold concentrated solutions in DMSO by serial 1:3.4 dilutions) in duplicate values for each concentration and IC₅₀ values are calculated.

AlphaScreen SureFire eIF4E Ser209 Phosphorylation Assay

The AlphaScreen SureFire eIF4E Ser209 phoshorylation assay can be used to measure the phosphorylation of endogenous eIF4E in cellular lysates. The AlphaScreen SureFire technology allows the detection of phosphorylated proteins in cellular lysates. In this assay, sandwich antibody complexes, which are only formed in the presence of the analyte (p-eIF4E Ser209), are captured by AlphaScreen donor and acceptor beads, bringing them into close proximity. The excitation of the donor bead provokes the release of singlet oxygen molecules that triggers a cascade of energy transfer in the Acceptor beads, resulting in the emission of light at 520-620 nm.

Surefire EIF4e Alphascreen in A549 Cells with 20% FCS Stimulation

For the assay the AlphaScreen SureFire p-eIF4E Ser209 10K Assay Kit and the AlphaScreen ProteinA Kit (for 10K assay points) both from Perkin Elmer are used.

On day one 50.000 A549 cells are plated in a 96-well plate in 100 μL per well in growth medium (DMEM/Hams' F12 with stable Glutamin, 10% FCS) and incubated at 37° C. After attachment of the cells, medium is changed to starving medium (DMEM, 0.1% FCS, without Glucose, with Glutamin, supplemented with 5 g/L Maltose). On day two, test compounds are serially diluted in 50 μL starving medium with a final DMSO concentration of 1% and are added to A549 cells in test plates at a final concentration range from as high 10 μM to as low 10 nM depending on the activities of the tested compounds. Treated cells are incubated at 37° C. for 2 h. 37 ul FCS is added to the wells (=final FCS concentration 20%) for 20 min. Then medium is removed and cells are lysed by adding 50 μL lysis buffer. Plates are then agitated on a plate shaker for 10 min. After 10 min lysis time, 4 μL of the lysate is transferred to a 384 well plate (Proxiplate from Perkin Elmer) and 5 μL Reaction Buffer plus Activation Buffer mix containing AlphaScreen Acceptor beads is added. Plates are sealed with TopSeal-A adhesive film, gently agitated on a plate shaker for 2 hours at room temperature. Afterwards 2 μL Dilution buffer with AlphaScreen Donor beads are added under subdued light and plates are sealed again with TopSeal-A adhesive film and covered with foil. Incubation takes place for further 2 h gently agitation at room temperature. Plates are then measured in an EnVision reader (Perkin Elmer) with the AlphaScreen program. Each data point (compound dilution) is measured as triplicate and 1050 values are calculated.

Proliferation Assays

The tumor cell proliferation assay which can be used to test the compounds of the present invention involves a readout called Cell Titer-Glow® Luminescent Cell Viability Assay developed by Promega® (B. A. Cunningham, “A Growing Issue: Cell Proliferation Assays, Modern kits ease quantification of cell growth”, The Scientist 2001, 15(13), 26; S. P. Crouch et al., “The use of ATP bioluminescence as a measure of cell proliferation and cytotoxicity”, Journal of Immunological Methods 1993, 160, 81-88), that measures inhibition of cell proliferation. Generation of a luminescent signal corresponds to the amount of ATP present, which is directly proportional to the number of metabolically active (proliferating) cells.

In Vitro Tumor Cell Proliferation Assay:

Cultivated tumour cells (MOLM-13 (human acute myeloid leukemia cells obtained from DSMZ # ACC 554), JJN-3 (human plasma cell leukemia cells obtained from DSMZ # ACC 541), Ramos (RA1) (human Burkitt's lymphoma cells obtained from ATCC # CRL-159)) are plated at a density of 2,500 cells/well (JJN-3), 3,000 cells/well (MOLM-13), 4,000 cells/well (Ramos (RA1)), in a 96-well multititer plate (Costar 3603 black/clear bottom) in 100 μL of their respective growth medium supplemented with 10% fetal calf serum. After 24 hours, the cells of one plate (zero-point plate) are measured for viability. Therefore, 70 μL/well CTG solution (Promega Cell Titer Glo solution (catalog # G755B and G756B)) is added to zero-point plate. The plates are mixed for two minutes on orbital shaker to ensure cell lysis and incubated for ten minutes at room temperature in the dark to stabilize luminescence signal. The samples are read on a VICTOR 3 plate reader. In parallel, serially test compounds are diluted in growth medium, and 50 μL of 3× dilutions/well are pipetted into the test plates (final concentrations: 0 μM, as well as in the range of 0.001-30 μM). The final concentration of the solvent dimethyl sulfoxide is 0.3-0.4%. The cells are incubated for 3 days in the presence of test substances. 105 μL/well CTG solution (Promega Cell Titer Glo solution (catalog # G755B and G756B)) is added to the test wells. The plates are mixed for 2 minutes on an orbital shaker to ensure cell lysis and incubated for 10 min at room temperature in the dark to stabilize luminescence signal. The samples are read on a VICTOR 3 plate reader. The change of cell number, in percent, is calculated by normalization of the measured values to the extinction values of the zero-point plate (=0%) and the extinction of the untreated (0 μm) cells (=100%).

Overview Cell Lines for Proliferation Assays

| | | Cell | |
|---|---|---|---|
| Cell line | Origin | number/well | Culture Medium |
| MOLM-13 (obtained | human | 3000 | RPMI 1640 with stable Glutamin |
| from DSMZ # ACC | acute | | with 10% Fetal Bovine Serum |
| 554) | myeloid | | |
| | leukemia | | |
| JJN-3 (obtained | human | 2500 | 45% Dulbecco's Modified Eagle |
| from DSMZ # ACC | plasma cell | | Medium with stable Glutamin, |
| 541) | leukemia | | 45% Iscove's Modified |
| | | | Dulbecco's Media with stable |
| | | | Glutamin and 10% Fetal Bovine |
| | | | Serum |
| Ramos (RA1) | human | 4000 | RPMI 1640 media with stable |
| (obtained from | Burkitt's | | Glutamin with 10% Fetal Bovine |
| ATCC # CRL-159) | lymphoma | | Serum |

Thus the compounds of the present invention effectively inhibit one or more kinases and are therefore suitable for the treatment or prophylaxis of diseases of uncontrolled cell growth, proliferation and/or survival, inappropriate cellular immune responses, or inappropriate cellular inflammatory responses, particularly in which the uncontrolled cell growth, proliferation and/or survival, inappropriate cellular immune responses, or inappropriate cellular inflammatory responses is mediated by MKNK, more particularly in which the diseases of uncontrolled cell growth, proliferation and/or survival, inappropriate cellular immune responses, or inappropriate cellular inflammatory responses are haemotological tumours, solid tumours and/or metastases thereof, e.g. leukaemias and myelodysplastic syndrome, malignant lymphomas, head and neck tumours including brain tumours and brain metastases, tumours of the thorax including non-small cell and small cell lung tumours, gastrointestinal tumours, endocrine tumours, mammary and other gynaecological tumours, urological tumours including renal, bladder and prostate tumours, skin tumours, and sarcomas, and/or metastases thereof.

1. A compound of general formula (I): 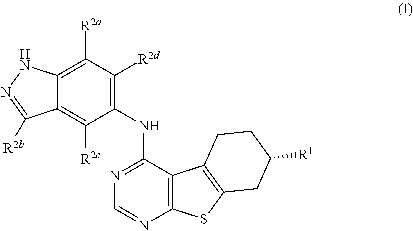
in which:
R¹ represents —C(═O)N(R³)R⁴;
R^{2a} represents a hydrogen atom;
R^{2b} represents a hydrogen atom;
R^{2c} represents a hydrogen atom or a halogen atom;
R^{2d} represents a hydrogen atom, a halogen atom, or a group selected from:
cyano-, —OR⁵, —SR⁶, —S(═O)₂R⁶, —S(═O)(═NH)R⁶, —N(H)R⁷, —N(R⁶)R⁷, —N(R⁶)R¹¹;
R³ represents a group selected from:
C₁-C₆-alkyl-, C₁-C₆-alkoxy-, C₃-C₆-alkenyl-, C₃-C₆-alkynyl-, —(CH₂)_{q}—(C₃-C₇-cycloalkyl), —(CH₂)ₚ—O—(C₃-C₇-cycloalkyl), —(CH₂)_{q}—(C₄-C₇-cycloalkenyl), —(CH₂)ₚ—O—(C₄-C₇-cycloalkenyl), —(CH₂)_{q}— (3- to 10-membered heterocycloalkyl), —(CH₂)ₚ—O— (3- to 10-membered heterocycloalkyl), —(CH₂)_{q}— (4- to 10-membered heterocycloalkenyl), —(CH₂)ₚ—O— (4- to 10-membered heterocycloalkenyl), —(CH₂)_{q}-aryl, —(CH₂)ₚ—O-aryl, —(CH₂)_{q}-heteroaryl, —(CH₂)ₚ—O-heteroaryl, —S(═O)₂—R⁶;
wherein said C₁-C₆-alkyl- group is optionally substituted one, two or three times, identically or differently, with a group selected from:
halo-, C₁-C₃-alkoxy-, HO—, —N(R⁸)R⁹;
R⁴ represents a C₁-C₄-alkyl- group;
wherein said C₁-C₄-alkyl- group is optionally substituted one, two or three times, identically or differently, with a halogen atom or a group selected from: HO—, C₁-C₃-alkoxy-, —CN, —N(R⁸)R⁹, —N(R⁷)R⁸, —C(═O)N(R⁸)R⁹, —C(═O)N(R⁷)R⁸;
or
N(R³)R⁴ together
represent a 3- to 10-membered heterocycloalkyl- or 4- to 10-membered heterocycloalkenyl group;
wherein said 3- to 10-membered heterocycloalkyl- group is optionally substituted one, two or three times, identically or differently, with a halogen atom or a group selected from: —(CH₂)_{q}—OH, —N(R⁷)R⁸, —N(R⁸)R⁹, C₁-C₃-alkyl-, —CN, —C(═O)R¹⁰, —C(═O)N(R⁸)R⁹, —(CH₂)_{q}-aryl, —(CH₂)_{q}-heteroaryl, —(C₁-C₃-alkyl)-N(R⁸)R⁹;
R⁵ represents a hydrogen atom or a group selected from:
C₁-C₅-alkyl-, —(CH₂)ₘ—(C₃-C₇-cycloalkyl), —(CH₂)ₘ— (3- to 10-membered heterocycloalkyl);
wherein said C₁-C₅-alkyl- group is optionally substituted one, two or three times, identically or differently, with a halogen atom or a group selected from: cyano, —N(R⁸)R⁹, —N(R⁸)C(═O)R¹⁰, -azido, phenyl-;
wherein said C₃-C₇-cycloalkyl- group and said 3- to 10-membered heterocycloalkyl-group are optionally substituted, one time, with a group selected from: cyano, —N(R⁸)R⁹, —C(═O)—O—R⁹;
R⁶ represents a hydrogen atom or a C₁-C₄-alkyl- group;
R⁷ represents a C₁-C₄-alkyl-, C₃-C₄-alkenyl- or C₁-C₃-alkoxy- group;
wherein said C₁-C₄-alkyl- is optionally substituted once with OH or —N(R⁸)R⁹;
or
N(R⁶)R⁷ together
represent a 3- to 10-membered heterocycloalkyl- or 4- to 10-membered heterocycloalkenyl group;
wherein said 3- to 10-membered heterocycloalkyl- group is optionally substituted once with —N(R⁸)R⁹;
R⁸ represents a hydrogen atom or a C₁-C₄-alkyl- group;
R⁹ represents a hydrogen atom or a C₁-C₆-alkyl- group;
or
N(R⁸)R⁹ together
represent a 3- to 10-membered heterocycloalkyl- group;
wherein said 3- to 10-membered heterocycloalkyl- group is optionally substituted one, two or three times, identically or differently, with a group selected from: halo-, —OH, —N(R⁷)R⁸, C₁-C₃-alkyl-;
R¹⁰ represents a (CH₂)ₘ—(C₃-C₇-cycloalkyl), C₁-C₆-alkyl- or a C₁-C₆-alkoxy- group;
R¹¹ represents a group selected from:
C₁-C₅-alkyl-, —(CH₂)ₙ—(C₃-C₇-cyclo alkyl), —(CH₂)ₙ— (3- to 10-membered heterocycloalkyl);
wherein said C₁-C₅-alkyl- group is optionally substituted one, two or three times, identically or differently, with a halogen atom or a group selected from: cyano, —N(R⁸)R⁹, —N(R⁸)C(═O)R¹⁹;
wherein said C₃-C₇-cycloalkyl- group and said 3- to 10-membered heterocycloalkyl-group are optionally substituted, one time, with a group selected from: cyano, —N(R⁸)R⁹, —C(═O)—O—R⁹;
m represent an integer of 0, 1 or 2;
n represent an integer of 0, 1 or 2;
p represents an integer of 2 or 3; and
q represents an integer of 0, 1, 2 or 3;
or a tautomer, an N-oxide, a hydrate, a solvate, or a salt thereof, or a mixture of same.

2. A compound according to claim 1, wherein:
R^{2d} represents a hydrogen atom, a halogen atom, or a group selected from: —OR⁵, —SR⁶, —S(═O)₂R⁶, —N(H)R⁷, —N(R⁶)R⁷;
or a tautomer, an N-oxide, a hydrate, a solvate, or a salt thereof, or a mixture of same.

3. A compound according to claim 1, wherein:
R^{2d} represents a hydrogen atom, a halogen atom, or a group selected from: —OR⁵, —SR⁶, —N(R⁶)R⁷;
or a tautomer, an N-oxide, a hydrate, a solvate, or a salt thereof, or a mixture of same.

4. A compound according to claim 1, wherein:
R^{2d} represents a C₁-C₃-alkoxy- group;
or a tautomer, an N-oxide, a hydrate, a solvate, or a salt thereof, or a mixture of same.

5. A compound according to claim 1, wherein:
R³ represents a group selected from:
C₁-C₆-alkyl-, C₁-C₃-alkoxy-, —(CH₂)_{q}—(C₃-C₇-cycloalkyl), —(CH₂)_{q}— (3- to 10-membered heterocycloalkyl), —(CH₂)_{q}-aryl, —(CH₂)_{q}-heteroaryl, —S(═O)₂—R⁶;
wherein said C₁-C₆-alkyl- group is optionally substituted one or two or three times, identically or differently, with a group selected from:
fluoro-, C₁-C₃-alkoxy-, HO—, —N(R⁸)R⁹;
R⁴ represents a C₁-C₃-alkyl- group;
wherein said C₁-C₃-alkyl- group is optionally substituted one, two or three times, identically or differently, with a group selected from:
fluoro-, HO—, C₁-C₃-alkoxy-, —N(R⁸)R⁹, —N(R⁷)R⁸, —C(═O)N(R⁸)R⁹,
or
N(R³)R⁴ together represent a 3- to 10-membered heterocycloalkyl- group; wherein said 3- to 10-membered heterocycloalkyl- group is optionally substituted one or two times, identically or differently, with a group selected from: fluoro-, —OH, —N(R⁷)R⁸, —N(R⁸)R⁹, C₁-C₃-alkyl-, —CN, —C(═O)N(R⁸)R⁹, -aryl, —(C₁-C₃-alkyl)-N(R⁸)R⁹;
or a tautomer, an N-oxide, a hydrate, a solvate, or a salt thereof, or a mixture of same.

6. A compound according to claim 1, wherein:
R⁵ represents a hydrogen atom or a C₁-C₅-alkyl-, C₃-C₇-cycloalkyl- or trifluoromethyl-group; wherein said C₁-C₅-alkyl- group is substituted once with a group selected from: —N(R⁸)R⁹, —N(R⁸)C(═O)R¹⁰, -azido;
or
R⁵ represents a
—(CH₂)ₘ— (3- to 10-membered heterocycloalkyl) group;
wherein said 3- to 10-membered heterocycloalkyl- group is optionally substituted, one time, with a group selected from: cyano, —N(R⁸)R⁹, —C(═O)—O—R⁹;
and
m represents an integer of 0 or 1;
or a tautomer, an N-oxide, a hydrate, a solvate, or a salt thereof, or a mixture of same.

7. A compound according to claim 1, wherein:
R⁶ represents a hydrogen atom or a C₁-C₄-alkyl- group; and
R⁷ represents a C₁-C₄-alkyl- or C₃-C₄-alkenyl- group;
wherein said C₁-C₄-alkyl- is optionally substituted once with OH or —N(R⁸)R⁹,
or
N(R⁶)R⁷ together represent a 3- to 7-membered heterocycloalkyl- group;
wherein said 3- to 7-membered heterocycloalkyl- group is optionally substituted once with —N(R⁸)R⁹;
or a tautomer, an N-oxide, a hydrate, a solvate, or a salt thereof, or a mixture of same.

8. A compound according to claim 1, wherein:
R⁸ represents a hydrogen atom or a C₁-C₂-alkyl- group; and
R⁹ represents a hydrogen atom or a C₁-C₂-alkyl- group;
or
N(R⁸)R⁹ together represent a 3- to 7-membered heterocycloalkyl- group;
or a tautomer, an N-oxide, a hydrate, a solvate, or a salt thereof, or a mixture of same.

9. A compound according to claim 1, wherein:
R¹ represents —C(═O)N(R³)R⁴;
R^{2a} represents a hydrogen atom;
R^{2b} represents a hydrogen atom;
R^{2c} represents a hydrogen atom;
R^{2d} represents a C₁-C₃-alkoxy- group;
N(R³)R⁴ together represent a 6-membered monocyclic heterocycloalkyl- group selected from piperidinyl-, piperazinyl- and morpholinyl-;
wherein said 6-membered monocyclic heterocycloalkyl- group is optionally substituted one or two times, identically or differently, with a group selected from: —N(CH₃)CH₃, methyl-;
or a tautomer, an N-oxide, a hydrate, a solvate, or a salt thereof, or a mixture of same.

10. A compound according to claim 1, which is selected from the group consisting of:
(7S)-4-(1H-indazol-5-ylamino)-N,N-dimethyl-5,6,7,8-tetrahydro[1]benzothieno[2,3-c]pyrimidine-7-carboxamide,
azetidin-1-yl[(7S)-4-(1H-indazol-5-ylamino)-5,6,7,8-tetrahydro[1]benzothieno[2,3-c]pyrimidin-7-yl]methanone,
(7S)—N-ethyl-4-(1H-indazol-5-ylamino)-N-methyl-5,6,7,8-tetrahydro[1]benzothieno[2,3-c]pyrimidine-7-carboxamide,
(7S)-4-(1H-indazol-5-ylamino)-N-methyl-N-(2,2,2-trifluoroethyl)-5,6,7,8-tetrahydro[1]benzothieno[2,3-c]pyrimidine-7-carboxamide,
(7S)-4-(1H-indazol-5-ylamino)-N-methyl-N-(3,3,3-trifluoropropyl)-5,6,7,8-tetrahydro[1]benzothieno[2,3-c]pyrimidine-7-carboxamide,
(7S)-4-(1H-indazol-5-ylamino)-N-methyl-N-(propan-2-yl)-5,6,7,8-tetrahydro[1]benzothieno[2,3-c]pyrimidine-7-carboxamide,
(7S)—N-ethyl-4-(1H-indazol-5-ylamino)-N-(propan-2-yl)-5,6,7,8-tetrahydro[1]benzothieno[2,3-c]pyrimidine-7-carboxamide,
[(7S)-4-(1H-indazol-5-ylamino)-5,6,7,8-tetrahydro[1]benzothieno[2,3-c]pyrimidin-7-yl](morpholin-4-yl)methanone,
[(7S)-4-(1H-indazol-5-ylamino)-5,6,7,8-tetrahydro[1]benzothieno[2,3-c]pyrimidin-7-yl][(1S,4S)-2-oxa-5-azabicyclo[2.2.1]hept-5-yl]methanone,
[(7S)-4-(1H-indazol-5-ylamino)-5,6,7,8-tetrahydro[1]benzothieno[2,3-d]pyrimidin-7-yl][(1R,4R)-2-oxa-5-azabicyclo[2.2.1]hept-5-yl]methanone,
[(7S)-4-(1H-indazol-5-ylamino)-5,6,7,8-tetrahydro[1]benzothieno[2,3-d]pyrimidin-7-yl](2-oxa-6-azaspiro[3.3]hept-6-yl)methanone,
(7S)-4-(1H-indazol-5-ylamino)-N-(2-methoxyethyl)-N-methyl-5,6,7,8-tetrahydro[1]benzothieno[2,3-d]pyrimidine-7-carboxamide,
[(7S)-4-(1H-indazol-5-ylamino)-5,6,7,8-tetrahydro[1]benzothieno[2,3-d]pyrimidin-7-yl](4-methylpiperazin-1-yl)methanone,
(7S)-4-(1H-indazol-5-ylamino)-N,N-bis(2-methoxyethyl)-5,6,7,8-tetrahydro[1]benzothieno[2,3-d]pyrimidine-7-carboxamide,
(3-hydroxy-3-methylazetidin-1-yl) [(7S)-4-(1H-indazol-5-ylamino)-5,6,7,8-tetrahydro[1]benzothieno[2,3-d]pyrimidin-7-yl]methanone,
(7S)—N-(2-hydroxy-2-methylpropyl)-4-(1H-indazol-5-ylamino)-N-methyl-5,6,7,8-tetrahydro[1]benzothieno[2,3-d]pyrimidine-7-carboxamide,
(7S)—N-ethyl-N-(2-hydroxyethyl)-4-(1H-indazol-5-ylamino)-5,6,7,8-tetrahydro[1]benzothieno[2,3-d]pyrimidine-7-carboxamide,
(7S)-4-[(6-methoxy-1H-indazol-5-yl)amino]-N,N-dimethyl-5,6,7,8-tetrahydro[1]benzothieno[2,3-d]pyrimidine-7-carboxamide,
azetidin-1-yl{(7S)-4-[(6-methoxy-1H-indazol-5-yl)amino]-5,6,7,8-tetrahydro[1]benzothieno[2,3-d]pyrimidin-7-yl}methanone,
{(7S)-4-[(6-methoxy-1H-indazol-5-yl)amino]-5,6,7,8-tetrahydro[1]benzothieno[2,3-d]pyrimidin-7-yl}(pyrrolidin-1-yl)methanone,
(7S)—N-ethyl-4-[(6-methoxy-1H-indazol-5-yl)amino]-N-methyl-5,6,7,8-tetrahydro[1]benzothieno[2,3-d]pyrimidine-7-carboxamide,
(7S)-4-[(6-methoxy-1H-indazol-5-yl)amino]-N-methyl-N-(propan-2-yl)-5,6,7,8-tetrahydro[1]benzothieno[2,3-d]pyrimidine-7-carboxamide
{(7S)-4-[(6-methoxy-1H-indazol-5-yl)amino]-5,6,7,8-tetrahydro[1]benzothieno[2,3-d]pyrimidin-7-yl}(morpholin-4-yl)methanone,
{(7S)-4-[(6-methoxy-1H-indazol-5-yl)amino]-5,6,7,8-tetrahydro[1]benzothieno[2,3-d]pyrimidin-7-yl}[(3S)-3-methylmorpholin-4-yl]methanone,
{(7S)-4-[(6-methoxy-1H-indazol-5-yl)amino]-5,6,7,8-tetrahydro[1]benzothieno[2,3-d]pyrimidin-7-yl}[(3R)-3-methylmorpholin-4-yl]methanone,
{(7S)-4-[(6-methoxy-1H-indazol-5-yl)amino]-5,6,7,8-tetrahydro[1]benzothieno[2,3-d]pyrimidin-7-yl}(2-oxa-6-azaspiro[3.3]hept-6-yl)methanone,
{(7S)-4-[(6-methoxy-1H-indazol-5-yl)amino]-5,6,7,8-tetrahydro[1]benzothieno[2,3-d]pyrimidin-7-yl}[(1S,4S)-2-oxa-5-azabicyclo[2.2.1]hept-5-yl]methanone,
{(7S)-4-[(6-methoxy-1H-indazol-5-yl)amino]-5,6,7,8-tetrahydro[1]benzothieno[2,3-d]pyrimidin-7-yl}[(1R,4R)-2-oxa-5-azabicyclo[2.2.1]hept-5-yl]methanone,
1-({(7S)-4-[(6-methoxy-1H-indazol-5-yl)amino]-5,6,7,8-tetrahydro[1]benzothieno[2,3-d]pyrimidin-7-yl}carbonyl)azetidine-3-carbonitrile,
(3-hydroxy-3-methylazetidin-1-yl) {(7S)-4-[(6-methoxy-1H-indazol-5-yl)amino]-5,6,7,8-tetrahydro[1]benzothieno[2,3-d]pyrimidin-7-yl}methanone,
(7S)—N-(2-hydroxyethyl)-4-[(6-methoxy-1H-indazol-5-yl)amino]-N-(oxetan-3-yl)-5,6,7,8-tetrahydro[1]benzothieno[2,3-d]pyrimidine-7-carboxamide,
(7S)—N-(2-hydroxyethyl)-4-[(6-methoxy-1H-indazol-5-yl)amino]-N-methyl-5,6,7,8-tetrahydro[1]benzothieno[2,3-d]pyrimidine-7-carboxamide,
(7S)—N-ethyl-N-(2-hydroxyethyl)-4-[(6-methoxy-1H-indazol-5-yl)amino]-5,6,7,8-tetrahydro[1]benzothieno[2,3-d]pyrimidine-7-carboxamide,
(7S)—N-(2-hydroxyethyl)-N-(2-methoxyethyl)-4-[(6-methoxy-1H-indazol-5-yl)amino]-5,6,7,8-tetrahydro[1]benzothieno[2,3-d]pyrimidine-7-carboxamide,
[3-(dimethylamino)azetidin-1-yl]{(7S)-4-[(6-methoxy-1H-indazol-5-yl)amino]-5,6,7,8-tetrahydro[1]benzothieno[2,3-d]pyrimidin-7-yl}methanone,
[(3S)-3-(dimethylamino)pyrrolidin-1-yl]{(7S)-4-[(6-methoxy-1H-indazol-5-yl)amino]-5,6,7,8-tetrahydro[1]benzothieno[2,3-d]pyrimidin-7-yl}methanone,
[4-(dimethylamino)piperidin-1-yl]{(7S)-4-[(6-methoxy-1H-indazol-5-yl)amino]-5,6,7,8-tetrahydro[1]benzothieno[2,3-d]pyrimidin-7-yl}methanone,
(4-{[2-(dimethylamino)ethyl](methyl)amino}piperidin-1-yl){(7S)-4-[(6-methoxy-1H-indazol-5-yl)amino]-5,6,7,8-tetrahydro[1]benzothieno[2,3-d]pyrimidin-7-yl}methanone,
{(7S)-4-[(6-methoxy-1H-indazol-5-yl)amino]-5,6,7,8-tetrahydro[1]benzothieno[2,3-d]pyrimidin-7-yl}(4-methylpiperazin-1-yl)methanone,
4-({(7S)-4-[(6-methoxy-1H-indazol-5-yl)amino]-5,6,7,8-tetrahydro[1]benzothieno [2,3-d]pyrimidin-7-yl}carbonyl)-N,N-dimethylpiperazine-1-carboxamide,
{4-[2-(dimethylamino)ethyl]piperazin-1-yl}{(7S)-4-[(6-methoxy-1H-indazol-5-yl)amino]-5,6,7,8-tetrahydro[1]benzothieno[2,3-d]pyrimidin-7-yl}methanone,
(7S)—N-[2-(dimethylamino)ethyl]-4-[(6-methoxy-1H-indazol-5-yl)amino]-N-methyl-5,6,7,8-tetrahydro[1]benzothieno[2,3-d]pyrimidine-7-carboxamide,
(7S)—N-[2-(dimethylamino)-2-oxoethyl]-4-[(6-methoxy-1H-indazol-5-yl)amino]-N-methyl-5,6,7,8-tetrahydro[1]benzothieno[2,3-d]pyrimidine-7-carboxamide,
(7S)—N-[2-(dimethylamino)ethyl]-N-ethyl-4-[(6-methoxy-1H-indazol-5-yl)amino]-5,6,7,8-tetrahydro[1]benzothieno[2,3-d]pyrimidine-7-carboxamide,
(7S)—N-[3-(dimethylamino)propyl]-4-[(6-methoxy-1H-indazol-5-yl)amino]-N-methyl-5,6,7,8-tetrahydro[1]benzothieno[2,3-d]pyrimidine-7-carboxamide,
(7S)—N-[3-(dimethylamino)-3-oxopropyl]-4-[(6-methoxy-1H-indazol-5-yl)amino]-N-methyl-5,6,7,8-tetrahydro[1]benzothieno[2,3-d]pyrimidine-7-carboxamide,
(7S)—N-cyclopropyl-N-[3-(dimethylamino)-3-oxopropyl]-4-[(6-methoxy-1H-indazol-5-yl)amino]-5,6,7,8-tetrahydro[1]benzothieno[2,3-d]pyrimidine-7-carboxamide,
(7S)—N-(2-{[2-(dimethylamino)ethyl](methyl)amino}ethyl)-4-[(6-methoxy-1H-indazol-5-yl)amino]-N-methyl-5,6,7,8-tetrahydro[1]benzothieno[2,3-d]pyrimidine-7-carboxamide,
(7S)—N-[3-(dimethylamino)propyl]-N-(2-hydroxyethyl)-4-[(6-methoxy-1H-indazol-5-yl)amino]-5,6,7,8-tetrahydro[1]benzothieno[2,3-d]pyrimidine-7-carboxamide,
(7S)—N,N-bis [3-(dimethylamino)propyl]-4-[(6-methoxy-1H-indazol-5-yl)amino]-5,6,7,8-tetrahydro[1]benzothieno[2,3-d]pyrimidine-7-carboxamide,
(7S)—N-[2-(dimethylamino)ethyl]-4-[(6-methoxy-1H-indazol-5-yl)amino]-N-(pyridin-2-ylmethyl)-5,6,7,8-tetrahydro[1]benzothieno[2,3-d]pyrimidine-7-carboxamide,
(7S)—N-[2-(dimethylamino)ethyl]-4-[(6-methoxy-1H-indazol-5-yl)amino]-N-(pyridin-3-ylmethyl)-5,6,7,8-tetrahydro[1]benzothieno[2,3-d]pyrimidine-7-carboxamide,
(7S)—N-[2-(dimethylamino)ethyl]-4-[(6-methoxy-1H-indazol-5-yl)amino]-N-(pyridin-4-ylmethyl)-5,6,7,8-tetrahydro[1]benzothieno[2,3-d]pyrimidine-7-carboxamide,
(7S)—N-benzyl-N-[3-(dimethylamino)propyl]-4-[(6-methoxy-1H-indazol-5-yl)amino]-5,6,7,8-tetrahydro[1]benzothieno[2,3-d]pyrimidine-7-carboxamide,
(4S,5R)-3-({(7S)-4-[(6-methoxy-1H-indazol-5-yl)amino]-5,6,7,8-tetrahydro[1]benzothieno[2,3-d]pyrimidin-7-yl}carbonyl)-4-methyl-5-phenyl-1,3-oxazolidin-2-one,
(7S)—N-(2,2-difluoroethyl)-4-[(6-ethoxy-1H-indazol-5-yl)amino]-N-methyl-5,6,7,8-tetrahydro[1]benzothieno[2,3-d]pyrimidine-7-carboxamide,
(7S)-4-[(6-ethoxy-1H-indazol-5-yl)amino]-N-methyl-N-(3,3,3-trifluoropropyl)-5,6,7,8-tetrahydro[1]benzothieno[2,3-d]pyrimidine-7-carboxamide,
{(7S)-4-[(6-ethoxy-1H-indazol-5-yl)amino]-5,6,7,8-tetrahydro[1]benzothieno[2,3-d]pyrimidin-7-yl}(pyrrolidin-1-yl)methanone,
1-({(7S)-4-[(6-ethoxy-1H-indazol-5-yl)amino]-5,6,7,8-tetrahydro[1]benzothieno[2,3-d]pyrimidin-7-yl}carbonyl)piperidin-4-one,
{(7S)-4-[(6-ethoxy-1H-indazol-5-yl)amino]-5,6,7,8-tetrahydro[1]benzothieno[2,3-d]pyrimidin-7-yl}[(1S,4S)-2-oxa-5-azabicyclo[2.2.1]hept-5-yl]methanone,
{(7S)-4-[(6-ethoxy-1H-indazol-5-yl)amino]-5,6,7,8-tetrahydro[1]benzothieno[2,3-d]pyrimidin-7-yl}[(1R,4R)-2-oxa-5-azabicyclo[2.2.1]hept-5-yl]methanone,
{(7S)-4-[(6-ethoxy-1H-indazol-5-yl)amino]-5,6,7,8-tetrahydro[1]benzothieno[2,3-d]pyrimidin-7-yl}(2-oxa-6-azaspiro [3.3]hept-6-yl)methanone,
(7S)-4-[(6-ethoxy-1H-indazol-5-yl)amino]-N-(2-hydroxyethyl)-N-methyl-5,6,7,8-tetrahydro[1]benzothieno[2,3-d]pyrimidine-7-carboxamide,
(7S)-4-[(6-ethoxy-1H-indazol-5-yl)amino]-N-(2-hydroxy-2-methylpropyl)-N-methyl-5,6,7,8-tetrahydro[1]benzothieno[2,3-d]pyrimidine-7-carboxamide,
[(3R,4R)-3,4-dihydroxypyrrolidin-1-yl]{(7S)-4-[(6-ethoxy-1H-indazol-5-yl)amino]-5,6,7,8-tetrahydro[1]benzothieno[2,3-d]pyrimidin-7-yl}methanone,
[(3S,4S)-3,4-dihydroxypyrrolidin-1-yl]{(7S)-4-[(6-ethoxy-1H-indazol-5-yl)amino]-5,6,7,8-tetrahydro[1]benzothieno[2,3-d]pyrimidin-7-yl}methanone,
(7S)-4-[(6-ethoxy-1H-indazol-5-yl)amino]-N,N-bis(2-hydroxyethyl)-5,6,7,8-tetrahydro[1]benzothieno[2,3-d]pyrimidine-7-carboxamide,
(7S)—N,N-dimethyl-4-{[6-(propan-2-yloxy)-1H-indazol-5-yl]amino}-5,6,7,8-tetrahydro[1]benzothieno[2,3-d]pyrimidine-7-carboxamide,
(7S)—N-ethyl-N-(propan-2-yl)-4-{[6-(propan-2-yloxy)-1H-indazol-5-yl]amino}-5,6,7,8-tetrahydro[1]benzothieno[2,3-d]pyrimidine-7-carboxamide,
(7S)—N-(2,2-difluoroethyl)-N-methyl-4-{[6-(propan-2-yloxy)-1H-indazol-5-yl]amino}-5,6,7,8-tetrahydro[1]benzothieno[2,3-d]pyrimidine-7-carboxamide,
[(7S)-4-{[6-(propan-2-yloxy)-1H-indazol-5-yl]amino}-5,6,7,8-tetrahydro[1]benzothieno [2,3-d]pyrimidin-7-yl](pyrrolidin-1-yl)methanone,
[(3S)-3-methylmorpholin-4-yl][(7S)-4-{[6-(propan-2-yloxy)-1H-indazol-5-yl]amino}-5,6,7,8-tetrahydro[1]benzothieno[2,3-d]pyrimidin-7-yl]methanone,
[(3R)-3-methylmorpholin-4-yl][(7S)-4-{[6-(propan-2-yloxy)-1H-indazol-5-yl]amino}-5,6,7,8-tetrahydro[1]benzothieno[2,3-d]pyrimidin-7-yl]methanone,
(1S,4S)-2-oxa-5-azabicyclo[2.2.1]hept-5-yl[(7S)-4-{[6-(propan-2-yloxy)-1H-indazol-5-yl]amino}-5,6,7,8-tetrahydro[1]benzothieno[2,3-d]pyrimidin-7-yl]methanone,
(1R,4R)-2-oxa-5-azabicyclo[2.2.1]hept-5-yl[(7S)-4-{[6-(propan-2-yloxy)-1H-indazol-5-yl]amino}-5,6,7,8-tetrahydro[1]benzothieno[2,3-d]pyrimidin-7-yl]methanone,
2-oxa-6-azaspiro[3.3]hept-6-yl[(7S)-4-{[6-(propan-2-yloxy)-1H-indazol-5-yl]amino}-5,6,7,8-tetrahydro[1]benzothieno[2,3-d]pyrimidin-7-yl]methanone,
(7S)—N-(2-hydroxyethyl)-N-methyl-4-{[6-(propan-2-yloxy)-1H-indazol-5-yl]amino}-5,6,7,8-tetrahydro[1]benzothieno[2,3-d]pyrimidine-7-carboxamide,
(7S)—N,N-bis(2-hydroxyethyl)-4-{[6-(propan-2-yloxy)-1H-indazol-5-yl]amino}-5,6,7,8-tetrahydro[1]benzothieno[2,3-d]pyrimidine-7-carboxamide,
[(3S,4S)-3,4-dihydroxypyrrolidin-1-yl][(7S)-4-{[6-(propan-2-yloxy)-1H-indazol-5-yl]amino}-5,6,7,8-tetrahydro[1]benzothieno[2,3-d]pyrimidin-7-yl]methanone,
[(3R,4R)-3,4-dihydroxypyrrolidin-1-yl][(7S)-4-{[6-(propan-2-yloxy)-1H-indazol-5-yl]amino}-5,6,7,8-tetrahydro[1]benzothieno[2,3-d]pyrimidin-7-yl]methanone,
(7S)—N,N-dimethyl-4-[(6-propoxy-1H-indazol-5-yl)amino]-5,6,7,8-tetrahydro[1]benzothieno[2,3-d]pyrimidine-7-carboxamide,
(3-hydroxy-3-methylazetidin-1-yl){(7S)-4-[(6-propoxy-1H-indazol-5-yl)amino]-5,6,7,8-tetrahydro[1]benzothieno[2,3-d]pyrimidin-7-yl}methanone,
(7S)-4-[(6-hydroxy-1H-indazol-5-yl)amino]-N,N-dimethyl-5,6,7,8-tetrahydro[1]benzothieno[2,3-d]pyrimidine-7-carboxamide,
(7S)-4-[(6-methoxy-1H-indazol-5-yl)amino]-N-methyl-N-propyl-5,6,7,8-tetrahydro[1]benzothieno[2,3-d]pyrimidine-7-carboxamide,
(7S)-4-{[6-(dimethylamino)-1H-indazol-5-yl]amino}-N,N-dimethyl-5,6,7,8-tetrahydro[1]benzothieno[2,3-d]pyrimidine-7-carboxamide,
(7S)—N,N-dimethyl-4-({6-[methyl(propan-2-yl)amino]-1H-indazol-5-yl}amino)-5,6,7,8-tetrahydro[1]benzothieno[2,3-d]pyrimidine-7-carboxamide,
(7S)-4-({6-[(2-hydroxy-2-methylpropyl)amino]-1H-indazol-5-yl}amino)-N,N-dimethyl-5,6,7,8-tetrahydro[1]benzothieno[2,3-d]pyrimidine-7-carboxamide,
(7S)-4-({6-[(2-hydroxy-2-methylpropyl)(methyl)amino]-1H-indazol-5-yl}amino)-N,N-dimethyl-5,6,7,8-tetrahydro[1]benzothieno[2,3-d]pyrimidine-7-carboxamide,
(7S)-4-{[6-(azetidin-1-yl)-1H-indazol-5-yl]amino}-N,N-dimethyl-5,6,7,8-tetrahydro[1]benzothieno[2,3-d]pyrimidine-7-carboxamide,
(7S)—N,N-dimethyl-4-{[6-(pyrrolidin-1-yl)-1H-indazol-5-yl]amino}-5,6,7,8-tetrahydro[1]benzothieno[2,3-d]pyrimidine-7-carboxamide,
(7S)-4-({6-[4-(dimethylamino)piperidin-1-yl]-1H-indazol-5-yl}amino)-N,N-dimethyl-5,6,7,8-tetrahydro[1]benzothieno[2,3-d]pyrimidine-7-carboxamide,
(7S)—N-butyl-4-({6-[4-(dimethylamino)piperidin-1-yl]-1H-indazol-5-yl}amino)-N-methyl-5,6,7,8-tetrahydro[1]benzothieno[2,3-d]pyrimidine-7-carboxamide,
(7S)—N,N-dimethyl-4-{[6-(methylsulfanyl)-1H-indazol-5-yl]amino}-5,6,7,8-tetrahydro[1]benzothieno[2,3-d]pyrimidine-7-carboxamide,
(7S)-4-{[6-(2-azidoethoxy)-1H-indazol-5-yl]amino}-N,N-dimethyl-5,6,7,8-tetrahydro[1]benzothieno[2,3-d]pyrimidine-7-carboxamide,
(7S)-4-{[6-(2-aminoethoxy)-1H-indazol-5-yl]amino}-N,N-dimethyl-5,6,7,8-tetrahydro[1]benzothieno[2,3-d]pyrimidine-7-carboxamide,
(7S)-4-({6-[2-(dimethylamino)ethoxy]-1H-indazol-5-yl}amino)-N,N-dimethyl-5,6,7,8-tetrahydro[1]benzothieno[2,3-d]pyrimidine-7-carboxamide,
(7S)-4-{[6-(3-azidopropoxy)-1H-indazol-5-yl]amino}-N,N-dimethyl-5,6,7,8-tetrahydro[1]benzothieno[2,3-d]pyrimidine-7-carboxamide,
(7S)-4-{[6-(3-aminopropoxy)-1H-indazol-5-yl]amino}-N,N-dimethyl-5,6,7,8-tetrahydro[1]benzothieno[2,3-d]pyrimidine-7-carboxamide,
(7S)—N,N-dimethyl-4-({6-[2-(pyrrolidin-1-yl)ethoxy]-1H-indazol-5-yl}amino)-5,6,7,8-tetrahydro[1]benzothieno[2,3-d]pyrimidine-7-carboxamide,
(7S)—N,N-dimethyl-4-({6-[2-(piperidin-1-yl)ethoxy]-1H-indazol-5-yl}amino)-5,6,7,8-tetrahydro[1]benzothieno[2,3-d]pyrimidine-7-carboxamide,
[(7S)-4-({6-[2-(dimethylamino)ethoxy]-1H-indazol-5-yl}amino)-5,6,7,8-tetrahydro[1]benzothieno[2,3-d]pyrimidin-7-yl][(3R)-3-methylmorpholin-4-yl]methanone,
[(3R)-3-methylmorpholin-4-yl][(7S)-4-({6-[2-(pyrrolidin-1-yl)ethoxy]-1H-indazol-5-yl}amino)-5,6,7,8-tetrahydro[1]benzothieno[2,3-d]pyrimidin-7-yl]methanone,
[(3R)-3-methylmorpholin-4-yl][(7S)-4-({6-[2-(piperidin-1-yl)ethoxy]-1H-indazol-5-yl}amino)-5,6,7,8-tetrahydro[1]benzothieno[2,3-d]pyrimidin-7-yl]methanone,
[(7S)-4-({6-[2-(dimethylamino)ethoxy]-1H-indazol-5-yl}amino)-5,6,7,8-tetrahydro[1]benzothieno[2,3-d]pyrimidin-7-yl][(3S)-3-methylmorpholin-4-yl]methanone,
[(3S)-3-methylmorpholin-4-yl][(7S)-4-({6-[2-(pyrrolidin-1-yl)ethoxy]-1H-indazol-5-yl}amino)-5,6,7,8-tetrahydro[1]benzothieno[2,3-d]pyrimidin-7-yl]methanone,
[(3S)-3-methylmorpholin-4-yl][(7S)-4-({6-[2-(piperidin-1-yl)ethoxy]-1H-indazol-5-yl}amino)-5,6,7,8-tetrahydro[1]benzothieno[2,3-d]pyrimidin-7-yl]methanone,
{(7S)-4-[(6-methoxy-1H-indazol-5-yl)amino]-5,6,7,8-tetrahydro[1]benzothieno [2,3-d]pyrimidin-7-yl}[(1S,4S)-5-methyl-2,5-diazabicyclo[2.2.1]hept-2-yl]methanone,
{(7S)-4-[(6-methoxy-1H-indazol-5-yl)amino]-5,6,7,8-tetrahydro[1]benzothieno [2,3-d]pyrimidin-7-yl}[(1R,4R)-5-methyl-2,5-diazabicyclo [2.2.1]hept-2-yl]methanone,
[4-(dimethylamino)piperidin-1-yl]{(7S)-4-[(6-ethoxy-1H-indazol-5-yl)amino]-5,6,7,8-tetrahydro[1]benzothieno[2,3-d]pyrimidin-7-yl}methanone,
(7S)-4-[(6-methoxy-1H-indazol-5-yl)amino]-N-methyl-N-(3,3,3-trifluoropropyl)-5,6,7,8-tetrahydro[1]benzothieno[2,3-d]pyrimidine-7-carboxamide,
(7S)—N-(2-hydroxy-2-methylpropyl)-4-[(6-methoxy-1H-indazol-5-yl)amino]-N-methyl-5,6,7,8-tetrahydro[1]benzothieno[2,3-d]pyrimidine-7-carboxamide,
(7S)—N-methyl-4-{[6-(propan-2-yloxy)-1H-indazol-5-yl]amino}-N-(3,3,3-trifluoropropyl)-5,6,7,8-tetrahydro[1]benzothieno[2,3-d]pyrimidine-7-carboxamide,
(7S)—N-(2-hydroxy-2-methylpropyl)-N-methyl-4-{[6-(propan-2-yloxy)-1H-indazol-5-yl]amino}-5,6,7,8-tetrahydro[1]benzothieno[2,3-d]pyrimidine-7-carboxamide,
(7S)-4-(1H-indazol-5-ylamino)-N,N-di(propan-2-yl)-5,6,7,8-tetrahydro[1]benzothieno[2,3-d]pyrimidine-7-carboxamide,
(7S)—N,N-dimethyl-4-({6-{[methyl(prop-2-en-1-yl)amino]-1H-indazol-5-yl}amino)-5,6,7,8-tetrahydro[1]benzothieno[2,3-d]pyrimidine-7-carboxamide,
(7S)-4-{[4-fluoro-6-(propan-2-yloxy)-1H-indazol-5-yl]amino}-N,N-dimethyl-5,6,7,8-tetrahydro[1]benzothieno[2,3-d]pyrimidine-7-carboxamide,
[(7S)-4-{[6-(2-aminoethoxy)-1H-indazol-5-yl]amino}-5,6,7,8-tetrahydro[1]benzothieno[2,3-d]pyrimidin-7-yl][(3R)-3-methylmorpholin-4-yl]methanone,
[(7S)-4-{[6-(2-aminoethoxy)-1H-indazol-5-yl]amino}-5,6,7,8-tetrahydro[1]benzothieno[2,3-d]pyrimidin-7-yl][(3S)-3-methylmorpholin-4-yl]methanone,
azetidin-1-yl[(7S)-4-({6-[4-(dimethylamino)piperidin-1-yl]-1H-indazol-5-yl}amino)-5,6,7,8-tetrahydro[1]benzothieno[2,3-d]pyrimidin-7-yl]methanone,
[(7S)-4-({6-[4-(dimethylamino)piperidin-1-yl]-1H-indazol-5-yl}amino)-5,6,7,8-tetrahydro[1]benzothieno[2,3-d]pyrimidin-7-yl](morpholin-4-yl)methanone,
[(7S)-4-({6-[2-(dimethylamino)ethoxy]-1H-indazol-5-yl}amino)-5,6,7,8-tetrahydro[1]benzothieno[2,3-d]pyrimidin-7-yl][4-(dimethylamino)piperidin-1-yl]methanone,
[(7S)-4-({6-[4-(dimethylamino)piperidin-1-yl]-1H-indazol-5-yl}amino)-5,6,7,8-tetrahydro[1]benzothieno[2,3-d]pyrimidin-7-yl][(3S)-3-methylmorpholin-4-yl]methanone,
[(7S)-4-({6-[4-(dimethylamino)piperidin-1-yl]-1H-indazol-5-yl}amino)-5,6,7,8-tetrahydro[1]benzothieno[2,3-d]pyrimidin-7-yl][(3R)-3-methylmorpholin-4-yl]methanone,
[(7S)-4-({6-[4-(dimethylamino)piperidin-1-yl]-1H-indazol-5-yl}amino)-5,6,7,8-tetrahydro[1]benzothieno[2,3-d]pyrimidin-7-yl][(2R,6S)-2,6-dimethylmorpholin-4-yl]methanone,
[4-(dimethylamino)piperidin-1-yl][(7S)-4-({6-[2-(pyrrolidin-1-yl)ethoxy]-1H-indazol-5-yl}amino)-5,6,7,8-tetrahydro[1]benzothieno[2,3-d]pyrimidin-7-yl}methanone,
[4-(dimethylamino)piperidin-1-yl][(7S)-4-({6-[2-(piperidin-1-yl)ethoxy]-1H-indazol-5-yl}amino)-5,6,7,8-tetrahydro[1]benzothieno[2,3-d]pyrimidin-7-yl}methanone,
{(7S)-4-[(6-{[(2R)-1-aminopropan-2-yl]oxy}-1H-indazol-5-yl)amino]-5,6,7,8-tetrahydro[1]benzothieno[2,3-d]pyrimidin-7-yl}[(3S)-3-methylmorpholin-4-yl]methanone,
(7S)-4-[(6-{[(2S)-1-aminopropan-2-yl]oxy}-1H-indazol-5-yl)amino]-N,N-dimethyl-5,6,7,8-tetrahydro[1]benzothieno[2,3-d]pyrimidine-7-carboxamide,
(7S)-4-[(6-{[(2S)-2-aminopropyl]oxy}-1H-indazol-5-yl)amino]-N,N-dimethyl-5,6,7,8-tetrahydro[1]benzothieno[2,3-d]pyrimidine-7-carboxamide,
{(7S)-4-[(6-{[(2S)-2-aminopropyl]oxy}-1H-indazol-5-yl)amino]-5,6,7,8-tetrahydro[1]benzothieno[2,3-d]pyrimidin-7-yl}[(3S)-3-methylmorpholin-4-yl]methanone,
{(7S)-4-[(6-{[(2R)-1-aminopropan-2-yl]oxy}-1H-indazol-5-yl)amino]-5,6,7,8-tetrahydro[1]benzothieno[2,3-d]pyrimidin-7-yl}[(3R)-3-methylmorpholin-4-yl]methanone
{(7S)-4-[(6-{[(2S)-1-aminopropan-2-yl]oxy}-1H-indazol-5-yl)amino]-5,6,7,8-tetrahydro[1]benzothieno[2,3-d]pyrimidin-7-yl}[(3S)-3-methylmorpholin-4-yl]methanone,
{(7S)-4-[(6-{[(2S)-2-aminopropyl]oxy}-1H-indazol-5-yl)amino]-5,6,7,8-tetrahydro[1]benzothieno[2,3-d]pyrimidin-7-yl}[(3R)-3-methylmorpholin-4-yl]methanone,
(7S)-4-[(6-{[(2R)-2-aminopropyl]oxy}-1H-indazol-5-yl)amino]-N,N-dimethyl-5,6,7,8-tetrahydro[1]benzothieno[2,3-d]pyrimidine-7-carboxamide,
{(7S)-4-[(6-{[(2S)-1-aminopropan-2-yl]oxy}-1H-indazol-5-yl)amino]-5,6,7,8-tetrahydro[1]benzothieno[2,3-d]pyrimidin-7-yl}[(3R)-3-methylmorpholin-4-yl]methanone,
{(7S)-4-[(6-{[(2R)-2-aminopropyl]oxy}-1H-indazol-5-yl)amino]-5,6,7,8-tetrahydro[1]benzothieno[2,3-d]pyrimidin-7-yl}[(3S)-3-methylmorpholin-4-yl]methanone,
{(7S)-4-[(6-{[(2R)-2-aminopropyl]oxy}-1H-indazol-5-yl)amino]-5,6,7,8-tetrahydro[1]benzothieno[2,3-d]pyrimidin-7-yl}[(3R)-3-methylmorpholin-4-yl]methanone,
(7S)-4-[(6-[2-(dimethylamino)ethyl](methyl)amino}-1H-indazol-5-yl)amino]-N,N-dimethyl-5,6,7,8-tetrahydro[1]benzothieno[2,3-d]pyrimidine-7-carboxamide,
(7S)-4-[(6-{[(2R)-1-aminopropan-2-yl]oxy}-1H-indazol-5-yl)amino]-N,N-dimethyl-5,6,7,8-tetrahydro[1]benzothieno[2,3-d]pyrimidine-7-carboxamide,
[(7S)-4-({6-[4-(dimethylamino)piperidin-1-yl]-1H-indazol-5-yl}amino)-5,6,7,8-tetrahydro[1]benzothieno[2,3-d]pyrimidin-7-yl][(1S,4S)-2-oxa-5-azabicyclo[2.2.1]hept-5-yl]methanone,
[(7S)-4-({6-[4-(dimethylamino)piperidin-1-yl]-1H-indazol-5-yl}amino)-5,6,7,8-tetrahydro[1]benzothieno[2,3-d]pyrimidin-7-yl][(1R,4R)-2-oxa-5-azabicyclo[2.2.1]hept-5-yl]methanone,
(7S)-4-({6-[4-(dimethylamino)piperidin-1-yl]-1H-indazol-5-yl}amino)-N-methyl-N-(propan-2-yl)-5,6,7,8-tetrahydro[1]benzothieno[2,3-d]pyrimidine-7-carboxamide,
(7S)-4-({6-[4-(dimethylamino)piperidin-1-yl]-1H-indazol-5-yl}amino)-N-methyl-N-propyl-5,6,7,8-tetrahydro[1]benzothieno[2,3-d]pyrimidine-7-carboxamide,
(7S)-4-({6-[4-(dimethylamino)piperidin-1-yl]-1H-indazol-5-yl}amino)-N-ethyl-N-(2-methoxyethyl)-5,6,7,8-tetrahydro[1]benzothieno[2,3-d]pyrimidine-7-carboxamide,
(7S)-4-({6-[4-(dimethylamino)piperidin-1-yl]-1H-indazol-5-yl}amino)-N-ethyl-N-methyl-5,6,7,8-tetrahydro[1]benzothieno[2,3-d]pyrimidine-7-carboxamide,
(3-hydroxyazetidin-1-yl) {(7S)-4-[(6-propoxy-1H-indazol-5-yl)amino]-5,6,7,8-tetrahydro[1]benzothieno[2,3-d]pyrimidin-7-yl}methanone,
[(2R,6S)-2,6-dimethylmorpholin-4-yl][(7S)-4-{[6-(propan-2-yloxy)-1H-indazol-5-yl]amino}-5,6,7,8-tetrahydro[1]benzothieno[2,3-d]pyrimidin-7-yl}methanone,
[(2R,6S)-2,6-dimethylmorpholin-4-yl]{(7S)-4-[(6-methoxy-1H-indazol-5-yl)amino]-5,6,7,8-tetrahydro[1]benzothieno[2,3-d]pyrimidin-7-yl}methanone,
[(2R,6S)-2,6-dimethylmorpholin-4-yl][(7S)-4-(1H-indazol-5-ylamino)-5,6,7,8-tetrahydro[1]benzothieno[2,3-d]pyrimidin-7-yl}methanone,
(3-fluoroazetidin-1-yl) {(7S)-4-[(6-methoxy-1H-indazol-5-yl)amino]-5,6,7,8-tetrahydro[1]benzothieno[2,3-d]pyrimidin-7-yl}methanone,
(3,3-difluoroazetidin-1-yl) {(7S)-4-[(6-methoxy-1H-indazol-5-yl)amino]-5,6,7,8-tetrahydro[1]benzothieno[2,3-d]pyrimidin-7-yl}methanone,
tert-butyl {2-[(5-{[(7S)-7-(dimethylcarbamoyl)-5,6,7,8-tetrahydro[1]benzothieno [2,3-d]pyrimidin-4-yl]amino}-1H-indazol-6-yl)oxy]ethyl}carbamate,
[(7S)-4-({6-[4-(dimethylamino)piperidin-1-yl]-1H-indazol-5-yl}amino)-5,6,7,8-tetrahydro[1]benzothieno[2,3-d]pyrimidin-7-yl](2-oxa-6-azaspiro[3.3]hept-6-yl)methanone,
[(2R,6S)-2,6-dimethylmorpholin-4-yl]{(7S)-4-[(6-ethoxy-1H-indazol-5-yl)amino]-5,6,7,8-tetrahydro[1]benzothieno[2,3-d]pyrimidin-7-yl}methanone,
[(3S)-3-(dimethylamino)pyrrolidin-1-yl][(7S)-4-{[6-(propan-2-yloxy)-1H-indazol-5-yl]amino}-5,6,7,8-tetrahydro[1]benzothieno[2,3-d]pyrimidin-7-yl]methanone,
[(3R)-3-(dimethylamino)pyrrolidin-1-yl][(7S)-4-{[6-(propan-2-yloxy)-1H-indazol-5-yl]amino}-5,6,7,8-tetrahydro[1]benzothieno[2,3-d]pyrimidin-7-yl]methanone,
(7S)—N-[3-(1H-imidazol-1-yl)propyl]-4-[(6-methoxy-1H-indazol-5-yl)amino]-N-methyl-5,6,7,8-tetrahydro[1]benzothieno[2,3-d]pyrimidine-7-carboxamide,
(7S)—N-[2-(1H-imidazol-1-yl)ethyl]-4-[(6-methoxy-1H-indazol-5-yl)amino]-N-methyl-5,6,7,8-tetrahydro[1]benzothieno[2,3-d]pyrimidine-7-carboxamide,
[(3S)-3-(dimethylamino)pyrrolidin-1-yl]{(7S)-4-[(6-ethoxy-1H-indazol-5-yl)amino]-5,6,7,8-tetrahydro[1]benzothieno[2,3-d]pyrimidin-7-yl}methanone,
(7S)—N-(2,2-dimethylpropyl)-4-[(6-ethoxy-1H-indazol-5-yl)amino]-N-methyl-5,6,7,8-tetrahydro[1]benzothieno[2,3-d]pyrimidine-7-carboxamide,
(7S)-4-[(6-ethoxy-1H-indazol-5-yl)amino]-N-methyl-N-propyl-5,6,7,8-tetrahydro[1]benzothieno[2,3-d]pyrimidine-7-carboxamide,
(7S)-4-[(6-ethoxy-1H-indazol-5-yl)amino]-5,6,7,8-tetrahydro[1]benzothieno[2,3-d]pyrimidin-7-yl}[3-(piperidin-1-yl)azetidin-1-yl]methanone,
(7S)-4-{[6-(2,2-dimethylpropoxy)-1H-indazol-5-yl]amino}-N,N-dimethyl-5,6,7,8-tetrahydro[1]benzothieno[2,3-d]pyrimidine-7-carboxamide,
(7S)—N-(2,2-dimethylpropyl)-4-[(6-methoxy-1H-indazol-5-yl)amino]-N-methyl-5,6,7,8-tetrahydro[1]benzothieno[2,3-d]pyrimidine-7-carboxamide,
{(7S)-4-[(6-methoxy-1H-indazol-5-yl)amino]-5,6,7,8-tetrahydro[1]benzothieno [2,3-d]pyrimidin-7-yl}[3-(piperidin-1-yl)azetidin-1-yl]methanone,
[(3R)-3-(dimethylamino)pyrrolidin-1-yl]{(7S)-4-[(6-methoxy-1H-indazol-5-yl)amino]-5,6,7,8-tetrahydro[1]benzothieno[2,3-d]pyrimidin-7-yl}methanone,
tert-butyl {3-[(5-{[(7S)-7-(dimethylcarbamoyl)-5,6,7,8-tetrahydro[1]benzothieno [2,3-d]pyrimidin-4-yl]amino}-1H-indazol-6-yl)oxy]propyl}carbamate,
(7S)-4-[(6-bromo-1H-indazol-5-yl)amino]-N,N-dimethyl-5,6,7,8-tetrahydro[1]benzothieno [2,3-d]pyrimidine-7-carboxamide,
(7S)-4-[(6-chloro-1H-indazol-5-yl)amino]-N,N-dimethyl-5,6,7,8-tetrahydro[1]benzothieno [2,3-d]pyrimidine-7-carboxamide,
[(2R,6S)-2,6-dimethylmorpholin-4-yl][(7S)-4-{[6-(pyrrolidin-1-yl)-1H-indazol-5-yl]amino}-5,6,7,8-tetrahydro[1]benzothieno[2,3-d]pyrimidin-7-yl}methanone,
[(3R)-3-methylmorpholin-4-yl][(7S)-4-{[6-(pyrrolidin-1-yl)-1H-indazol-5-yl]amino}-5,6,7,8-tetrahydro[1]benzothieno[2,3-d]pyrimidin-7-yl}methanone,
[(3S)-3-methylmorpholin-4-yl][(7S)-4-{[6-(pyrrolidin-1-yl)-1H-indazol-5-yl]amino}-5,6,7,8-tetrahydro[1]benzothieno[2,3-d]pyrimidin-7-yl}methanone,
(7S)—N,N-dimethyl-4-{[6-(methylsulfonyl)-1H-indazol-5-yl]amino}-5,6,7,8-tetrahydro[1]benzothieno[2,3-d]pyrimidine-7-carboxamide,
[(7S)-4-{[6-(dimethylamino)-1H-indazol-5-yl]amino}-5,6,7,8-tetrahydro[1]benzothieno [2,3-d]pyrimidin-7-yl][(3R)-3-methylmorpholin-4-yl]methanone,
[(7S)-4-{[6-(dimethylamino)-1H-indazol-5-yl]amino}-5,6,7,8-tetrahydro[1]benzothieno [2,3-d]pyrimidin-7-yl][(3S)-3-methylmorpholin-4-yl]methanone,
[(7S)-4-{[6-(dimethylamino)-1H-indazol-5-yl]amino}-5,6,7,8-tetrahydro[1]benzothieno [2,3-d]pyrimidin-7-yl][(2R,6S)-2,6-dimethylmorpholin-4-yl]methanone,
[(3R)-3-(dimethylamino)pyrrolidin-1-yl]{(7S)-4-[(6-ethoxy-1H-indazol-5-yl)amino]-5,6,7,8-tetrahydro[1]benzothieno[2,3-d]pyrimidin-7-yl}methanone,
(7S)—N,N-dimethyl-4-{[6-(trifluoromethoxy)-1H-indazol-5-yl]amino}-5,6,7,8-tetrahydro[1]benzothieno[2,3-d]pyrimidine-7-carboxamide,
(7S)—N,N-dimethyl-4-{[6-(propylsulfanyl)-1H-indazol-5-yl]amino}-5,6,7,8-tetrahydro[1]benzothieno[2,3-d]pyrimidine-7-carboxamide,
(7S)—N,N-dimethyl-4-({6-[2-(2-oxopyrrolidin-1-yl)ethoxy]-1H-indazol-5-yl}amino)-5,6,7,8-tetrahydro[1]benzothieno[2,3-d]pyrimidine-7-carboxamide,
(7S)—N,N-dimethyl-4-({6-[2-(2-oxo-1,3-oxazolidin-3-yl)ethoxy]-1H-indazol-5-yl}amino)-5,6,7,8-tetrahydro[1]benzothieno[2,3-d]pyrimidine-7-carboxamide,
azetidin-1-yl[(7S)-4-{[6-(pyrrolidin-1-yl)-1H-indazol-5-yl]amino}-5,6,7,8-tetrahydro[1]benzothieno[2,3-d]pyrimidin-7-yl]methanone,
azetidin-1-yl[(7S)-4-{[6-(dimethylamino)-1H-indazol-5-yl]amino}-5,6,7,8-tetrahydro[1]benzothieno[2,3-d]pyrimidin-7-yl]methanone,
(7S)—N-ethyl-N-methoxy-4-[(6-methoxy-1H-indazol-5-yl]amino}-5,6,7,8-tetrahydro[1]benzothieno[2,3-d]pyrimidine-7-carboxamide,
(7S)—N-methoxy-4-[(6-methoxy-1H-indazol-5-yl)amino]-N-methyl-5,6,7,8-tetrahydro[1]benzothieno[2,3-d]pyrimidine-7-carboxamide,
(7S)-4-{[6-(cyclopentyloxy)-1H-indazol-5-yl]amino}-N,N-dimethyl-5,6,7,8-tetrahydro[1]benzothieno[2,3-d]pyrimidine-7-carboxamide,
(7S)—N,N-dimethyl-4-{[6-(tetrahydro-2H-pyran-4-yloxy)-1H-indazol-5-yl]amino}-5,6,7,8-tetrahydro[1]benzothieno[2,3-d]pyrimidine-7-carboxamide,
(7S)—N,N-dimethyl-4-{[6-(tetrahydro-2H-pyran-4-ylmethoxy)-1H-indazol-5-yl]amino}-5,6,7,8-tetrahydro[1]benzothieno[2,3-d]pyrimidine-7-carboxamide,
(7S)-4-{[6-(cyclohexyloxy)-1H-indazol-5-yl]amino}-N,N-dimethyl-5,6,7,8-tetrahydro[1]benzothieno[2,3-d]pyrimidine-7-carboxamide,
(7S)-4-({6-[3-(dimethylamino)propoxy]-1H-indazol-5-yl}amino)-N,N-dimethyl-5,6,7,8-tetrahydro[1]benzothieno[2,3-d]pyrimidine-7-carboxamide,
(7S)-4-[(6-methoxy-1H-indazol-5-yl)amino]-N-(2-methoxy-2-methylpropyl)-N-methyl-5,6,7,8-tetrahydro[1]benzothieno[2,3-d]pyrimidine-7-carboxamide,
[(3RS)-3-fluoropiperidin-1-yl]{(7S)-4-[(6-methoxy-1H-indazol-5-yl)amino]-5,6,7,8-tetrahydro[1]benzothieno[2,3-d]pyrimidin-7-yl}methanone,
2,5-dihydro-1H-pyrrol-1-yl {(7S)-4-[(6-methoxy-1H-indazol-5-yl)amino]-5,6,7,8-tetrahydro[1]benzothieno[2,3-d]pyrimidin-7-yl}methanone,
{(7S)-4-[(6-methoxy-1H-indazol-5-yl)amino]-5,6,7,8-tetrahydro[1]benzothieno[2,3-d]pyrimidin-7-yl}(thiomorpholin-4-yl)methanone,
(3,3-difluoropyrrolidin-1-yl) {(7S)-4-[(6-methoxy-1H-indazol-5-yl)amino]-5,6,7,8-tetrahydro[1]benzothieno[2,3-d]pyrimidin-7-yl}methanone,
3-azabicyclo[3.1.0]hex-3-yl{(7S)-4-[(6-methoxy-1H-indazol-5-yl)amino]-5,6,7,8-tetrahydro[1]benzothieno[2,3-d]pyrimidin-7-yl}methanone,
[(2S,6S)-2,6-dimethylmorpholin-4-yl]{(7S)-4-[(6-methoxy-1H-indazol-5-yl)amino]-5,6,7,8-tetrahydro[1]benzothieno[2,3-d]pyrimidin-7-yl}methanone,
[(2R,6R)-2,6-dimethylmorpholin-4-yl]{(7S)-4-[(6-methoxy-1H-indazol-5-yl)amino]-5,6,7,8-tetrahydro[1]benzothieno[2,3-d]pyrimidin-7-yl}methanone,
{(7S)-4-[(6-methoxy-1H-indazol-5-yl)amino]-5,6,7,8-tetrahydro[1]benzothieno[2,3-d]pyrimidin-7-yl}(1,2-oxazinan-2-yl)methanone,
{(7S)-4-[(6-methoxy-1H-indazol-5-yl)amino]-5,6,7,8-tetrahydro[1]benzothieno[2,3-d]pyrimidin-7-yl}(1,2-oxazolidin-2-yl)methanone,
(7S)—N-ethoxy-4-[(6-methoxy-1H-indazol-5-yl)amino]-N-methyl-5,6,7,8-tetrahydro[1]benzothieno[2,3-d]pyrimidine-7-carboxamide,
(7S)—N-(3-hydroxy-3-methylbutyl)-4-[(6-methoxy-1H-indazol-5-yl)amino]-N-methyl-5,6,7,8-tetrahydro[1]benzothieno[2,3-d]pyrimidine-7-carboxamide,
(7S)-4-[(6-methoxy-1H-indazol-5-yl)amino]-N-methyl-N-(methylsulfonyl)-5,6,7,8-tetrahydro[1]benzothieno[2,3-d]pyrimidine-7-carboxamide,
(7S)—N-(2-methoxyethyl)-4-[(6-methoxy-1H-indazol-5-yl)amino]-N-methyl-5,6,7,8-tetrahydro[1]benzothieno[2,3-d]pyrimidine-7-carboxamide,
[3-(dimethylamino)azetidin-1-yl]{(7S)-4-[(6-ethoxy-1H-indazol-5-yl)amino]-5,6,7,8-tetrahydro[1]benzothieno[2,3-d]pyrimidin-7-yl}methanone,
[3-(dimethylamino)azetidin-1-yl][(7S)-4-{[6-(propan-2-yloxy)-1H-indazol-5-yl]amino}-5,6,7,8-tetrahydro[1]benzothieno[2,3-d]pyrimidin-7-yl}methanone,
3-azabicyclo[3.1.0]hex-3-yl{(7S)-4-[(6-fluoro-1H-indazol-5-yl)amino]-5,6,7,8-tetrahydro[1]benzothieno[2,3-d]pyrimidin-7-yl}methanone,
[(2R,6S)-2,6-dimethylmorpholin-4-yl]{(7S)-4-[(6-fluoro-1H-indazol-5-yl)amino]-5,6,7,8-tetrahydro[1]benzothieno[2,3-d]pyrimidin-7-yl}methanone,
(7S)-4-[(6-fluoro-1H-indazol-5-yl)amino]-N-(2-methoxyethyl)-N-methyl-5,6,7,8-tetrahydro[1]benzothieno[2,3-d]pyrimidine-7-carboxamide,
{(7S)-4-[(6-fluoro-1H-indazol-5-yl)amino]-5,6,7,8-tetrahydro[1]benzothieno[2,3-d]pyrimidin-7-yl}[(3R)-3-methylmorpholin-4-yl]methanone,
[(2R,6R)-2,6-dimethylmorpholin-4-yl]{(7S)-4-[(6-fluoro-1H-indazol-5-yl)amino]-5,6,7,8-tetrahydro[1]benzothieno[2,3-d]pyrimidin-7-yl}methanone,
[(2S,6S)-2,6-dimethylmorpholin-4-yl]{(7S)-4-[(6-fluoro-1H-indazol-5-yl)amino]-5,6,7,8-tetrahydro[1]benzothieno[2,3-d]pyrimidin-7-yl}methanone,
{(7S)-4-[(6-fluoro-1H-indazol-5-yl)amino]-5,6,7,8-tetrahydro[1]benzothieno[2,3-d]pyrimidin-7-yl}(morpholin-4-yl)methanone,
{(7S)-4-[(6-fluoro-1H-indazol-5-yl)amino]-5,6,7,8-tetrahydro[1]benzothieno[2,3-d]pyrimidin-7-yl}[(3S)-3-methylmorpholin-4-yl]methanone,
(7S)-4-[(6-fluoro-1H-indazol-5-yl)amino]-N,N-dimethyl-5,6,7,8-tetrahydro[1]benzothieno[2,3-d]pyrimidine-7-carboxamide,
{(7S)-4-[(6-chloro-1H-indazol-5-yl)amino]-5,6,7,8-tetrahydro[1]benzothieno[2,3-d]pyrimidin-7-yl}[(2S,6S)-2,6-dimethylmorpholin-4-yl]methanone,
(7S)-4-[(6-chloro-1H-indazol-5-yl)amino]-N-(2-methoxyethyl)-N-methyl-5,6,7,8-tetrahydro[1]benzothieno[2,3-d]pyrimidine-7-carboxamide,
{(7S)-4-[(6-chloro-1H-indazol-5-yl)amino]-5,6,7,8-tetrahydro[1]benzothieno[2,3-d]pyrimidin-7-yl}(morpholin-4-yl)methanone,
{(7S)-4-[(6-bromo-1H-indazol-5-yl)amino]-5,6,7,8-tetrahydro[1]benzothieno[2,3-d]pyrimidin-7-yl}[(3R)-3-methylmorpholin-4-yl]methanone,
{(7S)-4-[(6-bromo-1H-indazol-5-yl)amino]-5,6,7,8-tetrahydro[1]benzothieno[2,3-d]pyrimidin-7-yl}[(2R,6R)-2,6-dimethylmorpholin-4-yl]methanone,
{(7S)-4-[(6-bromo-1H-indazol-5-yl)amino]-5,6,7,8-tetrahydro[1]benzothieno[2,3-d]pyrimidin-7-yl}[(2R,6S)-2,6-dimethylmorpholin-4-yl]methanone,
{(7S)-4-[(6-bromo-1H-indazol-5-yl)amino]-5,6,7,8-tetrahydro[1]benzothieno[2,3-d]pyrimidin-7-y}[(3S)-3-methylmorpholin-4-yl]methanone,
{(7S)-4-[(6-bromo-1H-indazol-5-yl)amino]-5,6,7,8-tetrahydro[1]benzothieno[2,3-d]pyrimidin-7-yl}[(2S,6S)-2,6-dimethylmorpholin-4-yl]methanone,
{(7S)-4-[(6-bromo-1H-indazol-5-yl)aminol-5,6,7,8-tetrahydro[1]benzothieno[2,3-d]pyrimidin-7-yl}(morpholin-4-yl)methanone,
(7S)-4-[(6-bromo-1H-indazol-5-yl]amino}-N-(2-methoxyethyl)-N-methyl-5,6,7,8-tetrahydro[1]benzothieno[2,3-d]pyrimidine-7-carboxamide,
[(7S)-4-{[6-(dimethylamino)-1H-indazol-5-yl]amino}-5,6,7,8-tetrahydro[1]benzothieno[2,3-d]pyrimidin-7-yl][(2S,6S)-2,6-dimethylmorpholin-4-yl]methanone,
[(7S)-4-{[6-(dimethylamino)-1H-indazol-5-yl]amino}-5,6,7,8-tetrahydro[1]benzothieno[2,3-d]pyrimidin-7-yl][(2R,6R)-2,6-dimethylmorpholin-4-yl]methanone,
(7S)-4-{[6-(dimethylamino)-1H-indazol-5-yl]amino}-N-methyl-N-(3,3,3-trifluoropropyl)-5,6,7,8-tetrahydro[1]benzothieno[2,3-d]pyrimidine-7-carboxamide,
(7S)-4-({6-[(3-hydroxy-3-methylbutyl)(methyl)amino]-1H-indazol-5-yl}amino)-N,N-dimethyl-5,6,7,8-tetrahydro[1]benzothieno[2,3-d]pyrimidine-7-carboxamide,
tert-butyl {2-[(5-{[(7S)-7-(dimethylcarbamoyl)-5,6,7,8-tetrahydro[1]benzothieno [2,3-d]pyrimidin-4-yl]amino}-1H-indazol-6-yl)(methyl)amino]ethyl}carbamate,
tert-butyl {2-[(5-{[(7S)-7-(dimethylcarbamoyl)-5,6,7,8-tetrahydro[1]benzothieno [2,3-d]pyrimidin-4-yl]amino}-1H-indazol-6-yl)amino]ethyl}carbamate,
(7S)—N-methyl-4-{[6-(pyrrolidin-1-yl)-1H-indazol-5-yl]amino}-N-(3,3,3-trifluoropropyl)-5,6,7,8-tetrahydro[1]benzothieno[2,3-d]pyrimidine-7-carboxamide,
[(2S,6S)-2,6-dimethylmorpholin-4-yl][(7S)-4-{[6-(methylsulfanyl)-1H-indazol-5-yl]amino}-5,6,7,8-tetrahydro[1]benzothieno[2,3-d]pyrimidin-7-yl}methanone,
[(2R,6R)-2,6-dimethylmorpholin-4-yl][(7S)-4-{[6-(methylsulfanyl)-1H-indazol-5-yl]amino}-5,6,7,8-tetrahydro[1]benzothieno[2,3-d]pyrimidin-7-yl}methanone,
[(3R)-3-methylmorpholin-4-yl][(7S)-4-{[6-(methylsulfanyl)-1H-indazol-5-yl]amino}-5,6,7,8-tetrahydro[1]benzothieno[2,3-d]pyrimidin-7-yl}methanone,
(7S)—N-methyl-4-{[6-(methylsulfanyl)-1H-indazol-5-yl]amino}-N-(3,3,3-trifluoropropyl)-5,6,7,8-tetrahydro[1]benzothieno[2,3-d]pyrimidine-7-carboxamide,
azetidin-1-yl[(7S)-4-{[6-(methylsulfanyl)-1H-indazol-5-yl]amino}-5,6,7,8-tetrahydro[1]benzothieno[2,3-d]pyrimidin-7-yl]methanone,
(7S)—N-methyl-4-{[6-(methylsulfanyl)-1H-indazol-5-yl]amino}-N-(propan-2-yl)-5,6,7,8-tetrahydro[1]benzothieno[2,3-d]pyrimidine-7-carboxamide,
[(2R,6S)-2,6-dimethylmorpholin-4-yl][(7S)-4-{[6-(methylsulfanyl)-1H-indazol-5-yl]amino}-5,6,7,8-tetrahydro[1]benzothieno[2,3-d]pyrimidin-7-yl}methanone,
(7S)—N-(2-methoxyethyl)-N-methyl-4-{[6-(methylsulfanyl)-1H-indazol-5-yl]amino}-5,6,7,8-tetrahydro[1]benzothieno[2,3-d]pyrimidine-7-carboxamide,
[(3S)-3-methylmorpholin-4-yl][(7S)-4-{[6-(methylsulfanyl)-1H-indazol-5-yl]amino}-5,6,7,8-tetrahydro[1]benzothieno[2,3-d]pyrimidin-7-yl]methanone,
[(7S)-4-{[6-(methylsulfanyl)-1H-indazol-5-yl]amino}-5,6,7,8-tetrahydro[1]benzothieno [2,3-d]pyrimidin-7-yl](morpholin-4-yl)methanone,
tert-butyl {2-[(5-{[(7S)-7-(dimethylcarbamoyl)-5,6,7,8-tetrahydro[1]benzothieno [2,3-d]pyrimidin-4-yl]amino}-1H-indazol-6-yl)oxy]ethyl}methylcarbamate,
(7S)-4-[(6-{2-[(2,2-dimethylpropanoyl)amino]ethoxy}-1H-indazol-5-yl)amino]-N,N-dimethyl-5,6,7,8-tetrahydro[1]benzothieno[2,3-d]pyrimidine-7-carboxamide,
methyl {2-[(5-{[(7S)-7-(dimethylcarbamoyl)-5,6,7,8-tetrahydro[1]benzothieno[2,3-d]pyrimidin-4-yl]amino}-1H-indazol-6-yl)oxy]ethyl}carbamate,
tert-butyl {(2R)-2-[(5-{[(7S)-7-(dimethylcarbamoyl)-5,6,7,8-tetrahydro[1]benzothieno [2,3-d]pyrimidin-4-yl]amino}-1H-indazol-6-yl)oxy]propyl}carbamate,
propan-2-yl {2-[(5-{[(7S)-7-(dimethylcarbamoyl)-5,6,7,8-tetrahydro[1]benzothieno [2,3-d]pyrimidin-4-yl]amino}-1H-indazol-6-yl)oxy]ethyl}carbamate,
propan-2-yl {(2R)-2-[(5-{[(7S)-7-(dimethylcarbamoyl)-5,6,7,8-tetrahydro[1]benzothieno [2,3-d]pyrimidin-4-yl]amino}-1H-indazol-6-yl)oxy]propyl}carbamate,
ethyl {2-[(5-{[(7S)-7-(dimethylcarbamoyl)-5,6,7,8-tetrahydro[1]benzothieno[2,3-d]pyrimidin-4-yl]amino}-1H-indazol-6-yl)oxy]ethyl}carbamate,
ethyl {(2R)-2-[(5-{[(7S)-7-(dimethylcarbamoyl)-5,6,7,8-tetrahydro[1]benzothieno [2,3-d]pyrimidin-4-yl]amino}-1H-indazol-6-yl)oxy]propyl}carbamate
methyl {(2R)-2-[(5-{[(7S)-7-(dimethylcarbamoyl)-5,6,7,8-tetrahydro[1]benzothieno [2,3-d]pyrimidin-4-yl]amino}-1H-indazol-6-yl)oxy]propyl}carbamate,
tert-butyl 3-[(5-{[(7S)-7-(dimethylcarbamoyl)-5,6,7,8-tetrahydro[1]benzothieno [2,3-d]pyrimidin-4-yl]amino}-1H-indazol-6-yl)oxy]azetidine-1-carboxylate,
tert-butyl 3-[(5-{[(7S)-7-{[(2R,6S)-2,6-dimethylmorpholin-4-yl]carbonyl}-5,6,7,8-tetrahydro[1]benzothieno[2,3-d]pyrimidin-4-yl]amino}-1H-indazol-6-yl)oxy]azetidine-1-carboxylate,
tert-butyl 3-[(5-{[(7S)-7-{[(3 S)-3-methylmorpholin-4-yl]carbonyl}-5,6,7,8-tetrahydro[1]benzothieno[2,3-d]pyrimidin-4-yl]amino}-1H-indazol-6-yl)oxy]azetidine-1-carboxylate,
[(7S)-4-{[6-(azetidin-3-yloxy)-1H-indazol-5-yl]amino}-5,6,7,8-tetrahydro[1]benzothieno [2,3-d]pyrimidin-7-yl][(3S)-3-methylmorpholin-4-yl]methanone,
(7S)—N,N-dimethyl-4-({6-[2-(propanoylamino)ethoxy]-1H-indazol-5-yl}amino)-5,6,7,8-tetrahydro[1]benzothieno[2,3-d]pyrimidine-7-carboxamide,
(7S)-4-({6-[2-(butanoylamino)ethoxy]-1H-indazol-5-yl}amino)-N,N-dimethyl-5,6,7,8-tetrahydro[1]benzothieno[2,3-d]pyrimidine-7-carboxamide,
(7S)—N,N-dimethyl-4-[(6-{2-[(3-methylbutanoyl)amino]ethoxy}-1H-indazol-5-yl)amino]-5,6,7,8-tetrahydro[1]benzothieno[2,3-d]pyrimidine-7-carboxamide,
(7S)-4-[(6-{2-[(3,3-dimethylbutanoyl)amino]ethoxy}-1H-indazol-5-yl)amino]-N,N-dimethyl-5,6,7,8-tetrahydro[1]benzothieno[2,3-d]pyrimidine-7-carboxamide,
(7S)-4-[(6-{2-[(cyclopentylacetyl)amino]ethoxy}-1H-indazol-5-yl)amino]-N,N-dimethyl-5,6,7,8-tetrahydro[1]benzothieno[2,3-d]pyrimidine-7-carboxamide,
(7S)-4-[(6-{2-[(cyclohexylacetyl)amino]ethoxy}-1H-indazol-5-yl)amino]-N,N-dimethyl-5,6,7,8-tetrahydro[1]benzothieno[2,3-d]pyrimidine-7-carboxamide,
(7S)-4-[(6-{2-[(2,2-dimethylpropyl)amino]ethoxy}-1H-indazol-5-yl)amino]-N,N-dimethyl-5,6,7,8-tetrahydro[1]benzothieno[2,3-d]pyrimidine-7-carboxamide,
(7S)-4-({6-[3-(3-fluoroazetidin-1-yl)propoxy]-1H-indazol-5-yl}amino)-N,N-dimethyl-5,6,7,8-tetrahydro[1]benzothieno[2,3-d]pyrimidine-7-carboxamide,
(7S)—N,N-dimethyl-4-({6-[3-(pyrrolidin-1-yl)propoxy]-1H-indazol-5-yl}amino)-5,6,7,8-tetrahydro[1]benzothieno[2,3-d]pyrimidine-7-carboxamide,
(7S)—N,N-dimethyl-4-({6-[3-(piperidin-1-yl)propoxy]-1H-indazol-5-yl}amino)-5,6,7,8-tetrahydro[1]benzothieno[2,3-d]pyrimidine-7-carboxamide,
[(7S)-4-{[6-(2,2-dimethylpropoxy)-1H-indazol-5-yl]amino}-5,6,7,8-tetrahydro[1]benzothieno[2,3-d]pyrimidin-7-yl](morpholin-4-yl)methanone,
(7S)—N,N-dimethyl-4-({6-[(3S)-tetrahydrofuran-3-yloxy]-1H-indazol-5-yl}amino)-5,6,7,8-tetrahydro[1]benzothieno[2,3-d]pyrimidine-7-carboxamide,
(7S)-4-[(6-methoxy-1H-indazol-5-yl)amino]-N-[(2R)-2-methoxypropyl]-N-methyl-5,6,7,8-tetrahydro[1]benzothieno[2,3-d]pyrimidine-7-carboxamide,
(7S)-4-[(6-methoxy-1H-indazol-5-yl)amino]-N-[(2S)-2-methoxypropyl]-N-methyl-5,6,7,8-tetrahydro[1]benzothieno[2,3-d]pyrimidine-7-carboxamide,
(7S)-4-[(6-methoxy-1H-indazol-5-yl)amino]-N-[(2S)-1-methoxypropan-2-yl]-N-methyl-5,6,7,8-tetrahydro[1]benzothieno[2,3-d]pyrimidine-7-carboxamide,
(7S)-4-[(6-methoxy-1H-indazol-5-yl)amino]-N-[(2R)-1-methoxypropan-2-yl]-N-methyl-5,6,7,8-tetrahydro[1]benzothieno[2,3-d]pyrimidine-7-carboxamide,
[3-(dimethylamino)azetidin-1-yl][(7S)-4-{[6-(methylsulfanyl)-1H-indazol-5-yl]amino}-5,6,7,8-tetrahydro[1]benzothieno[2,3-d]pyrimidin-7-yl]methanone,
or a tautomer, an N-oxide, a hydrate, a solvate, or a salt thereof, or a mixture of same.

11. A method of preparing a compound of general formula (I) according to claim 1, comprising reacting an intermediate compound of general formula (VII): 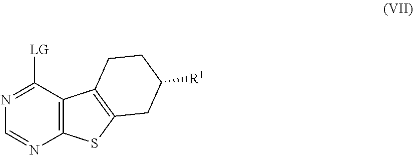
in which R¹ is as defined in claim 1, and LG represents a leaving group;
with a compound of general formula (II): 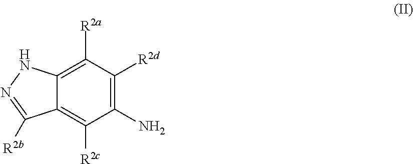
in which R^{2a}, R^{2b}, R^{2c}, and R^{2d} are as defined in claim 1;
thus providing a compound of general formula (I): 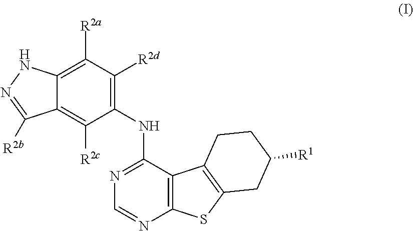
in which R¹, R^{2a}, R^{2b}, R^{2c}, and R^{2d} are as defined in claim 1.

12. A method of preparing a compound of general formula (I) according to claim 1, comprising reacting an intermediate compound of general formula (V): 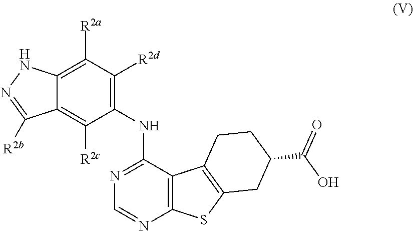
in which R^{2a}, R^{2b}, R^{2c}, and R^{2d} are as defined in claim 1;
with a compound of general formula (VI):
R³R⁴—NH (VI)
in which R³ and R⁴ are as defined in claim 1;
thus providing a compound of general formula (I): 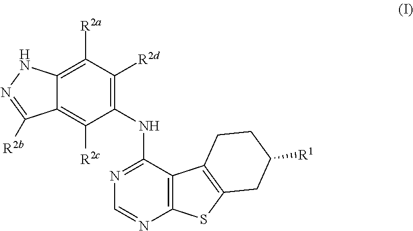
in which R¹, R^{2a}, R^{2b}, R^{2c}, and R^{2d} are as defined in claim 1.

13. (canceled)

14. A pharmaceutical composition comprising a compound of general formula (I), or a tautomer, an N-oxide, a hydrate, a solvate, or a pharmaceutically acceptable salt thereof, or a mixture of same, according to claim 1, and a pharmaceutically acceptable diluent or carrier.

15. A pharmaceutical combination comprising:
one or more first active ingredients selected from a compound of general formula (I) according to claim 1, and
one or more second active ingredients selected from chemotherapeutic anti-cancer agents.

16. A method for the treatment of a disease comprising administering to a patient in need thereof a therapeutically effective amount of a compound of general formula (I), or a tautomer, an N-oxide, a hydrate, a solvate, or a pharmaceutically acceptable salt thereof, or a mixture of same, according to claim 1.

17. (canceled)

18. The method according to claim 16, wherein said disease is a disease of uncontrolled cell growth, proliferation or survival, an inappropriate cellular immune response, or an inappropriate cellular inflammatory response.

19. A compound of general formula (VII): 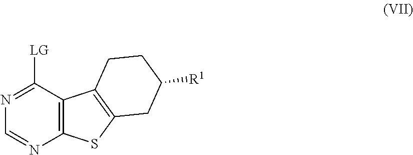
in which R¹ is as defined in claim 1, and LG represents a leaving group.

20. (canceled)

21. A compound of general formula (V): 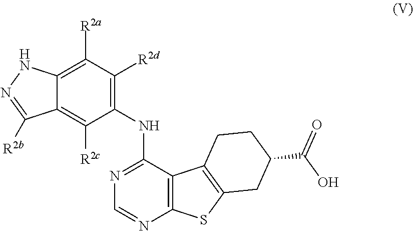
in which R¹ is as defined in claim 1, and LG represents a leaving group.

22. (canceled)

23. The method according to claim 18, wherein the uncontrolled cell growth, proliferation or survival, inappropriate cellular immune response, or inappropriate cellular inflammatory response is mediated by the MKNK-1 pathway.


SEQUENCE LISTING (19 sequences)

SEQ ID NO: 1 - protein, 14 aa, Unknown
  biotin-Ahx-IKKRKLTRRKSLKG
IKKRKLTRRKSLKG

SEQ ID NO: 2 - protein, 14 aa, Unknown
  biotin-Ahx-IKKRKLTRRKSLKG
IKKRKLTRRKSLKG

SEQ ID NO: 3 - protein, 14 aa, Unknown
  biotin-Ahx-AEEEEYFELVAKKK
AEEEEYFELVAKKK

SEQ ID NO: 4 - protein, 14 aa, Unknown
  biotin-Ttds-YISPLKSPYKISEG
YISPLKSPYKISEG

SEQ ID NO: 5 - protein, 13 aa, Unknown
  biotin-KVEKIGEGTYGVV
KVEKIGEGTYGVV

SEQ ID NO: 6 - protein, 15 aa, Unknown
  Biotin-Ahx-GGEEEEYFELVKKKK
GGEEEEYFELVKKKK

SEQ ID NO: 7 - protein, 12 aa, Unknown
  biotin-Ahx-VLLPKKSFAEPG
VLLPKKSFAEPG

SEQ ID NO: 8 - protein, 13 aa, Unknown
  biotin-Ahx-KKLNRTLSFAEPG
KKLNRTLSFAEPG

SEQ ID NO: 9 - protein, 12 aa, Unknown
  biotin-Ahx-GDEDFSSFAEPG
GDEDFSSFAEPG

SEQ ID NO: 10 - protein, 15 aa, Unknown
  biotin-Ahx-GGEEEEYFELVKKKK
GGEEEEYFELVKKKK

SEQ ID NO: 11 - protein, 12 aa, Unknown
  biotin-Ttds-YRRRHLSFAEPG
YRRRHLSFAEPG

SEQ ID NO: 12 - protein, 15 aa, Unknown
  biotin-Ahx-GGEEEEYFELVKKKK
GGEEEEYFELVKKKK

SEQ ID NO: 13 - protein, 14 aa, Unknown
  biotin-Ahx-AAEEEYFFLFAKKK
AAEEEYFFLFAKKK

SEQ ID NO: 14 - protein, 27 aa, Unknown
  biotin-Ahx-YRRAAVPPSPSLSRHSSPHQ-pS-EDEEE
YRRAAVPPSPSLSRHSSPHQPSEDEEE

SEQ ID NO: 15 - protein, 12 aa, Unknown
  biotin-Ahx-HRGLRASFAEPG
HRGLRASFAEPG

SEQ ID NO: 16 - protein, 17 aa, Unknown
  biotin-Ahx-DFGLARDMYDKEYYSVG
DFGLARDMYDKEYYSVG

SEQ ID NO: 17 - protein, 14 aa, Unknown
  biotin-Ahx-PWDPDDADITEILG
PWDPDDADITEILG

SEQ ID NO: 18 - protein, 14 aa, Unknown
  biotin-Ahx-PWDPDDADITEILG
PWDPDDADITEILG

SEQ ID NO: 19 - protein, 14 aa, Unknown [sequence omitted]